(19) United States
(12) Patent Application Publication
(10) Pub. No.: US 2026/0216347 A1
(43) Pub. Date: Jul. 30, 2026

(54) Substituted Imidazo-Based Compounds as Ligand Directed Degraders of IRAK3

(71) Applicant: Celgene Corporation, Princeton, NJ (US)

(72) Inventors: Farid van der Mei, Cambridge, MA (US); Guobin Miao, Lexington, MA (US); Laura Akullian D'Agostino, Sudbury, MA (US); Rulin Ma, Winchester, MA (US); Kurt Armbrust, Cambridge, MA (US)

(73) Assignee: Celgene Corporation, Princeton, NJ (US)

(21) Appl. No.: 19/139,476

(22) PCT Filed: Dec. 20, 2023
(86) PCT No.: PCT/US23/85013

§ 371 Date: Jun. 16, 2025

Related U.S. Application Data
(60) Provisional application No. 63/434,197, filed on Dec. 21, 2022.

(51) Int. Cl. A61K 47/55; A61K 47/54

(52) U.S. Cl. CPC A61K 47/55; A61K 47/545

(57) ABSTRACT

Provided herein are compounds and compositions thereof for modulating IRAK3. In some embodiments, the compounds and compositions are provided for treatment of cancer.

### CROSS-REFERENCE TO RELATED APPLICATIONS

This application claims priority to U.S. Provisional Application No. 63/434,197, filed on Dec. 21, 2022, which is incorporated herein by reference in its entirety for any purpose.

### FIELD

The present disclosure relates generally to compounds, compositions, and methods for their preparation and use of the compounds and compositions for treating cancer.

### BACKGROUND

The recruitment of immune cells to sites of injury involves the concerted interactions of a large number of soluble mediators. Several cytokines appear to play key roles in these processes, including interleukin-1 (IL-1). IL-1 produces proinflammatory responses and contributes to the tissue degeneration observed in chronic inflammatory conditions. IL-1 has also been implicated in the process of bone resorption and adipose tissue regulation. Thus, IL-1 plays a key role in a large number of pathological conditions including rheumatoid arthritis, inflammatory bowel disease, multiple sclerosis, diabetes, obesity, cancer, and sepsis.

IL-1 treatment of cells induces the formation of a complex consisting of the two IL-1 receptor chains, IL-1R1 and IL-1RAcP, and the resulting heterodimer recruits an adaptor molecule designated as MyD88, which binds to IL-1 receptor associated kinase (IRAK) (Wesche et al., J. Biol. Chem. 1999, 274, 19403-19410; O'Neill et al., J. Leukoc. Biol. 1998, 63, 650-657; Auron, Cytokine Growth Factor Rev. 1998, 9:221-237; and O'Neill, Biochem. Soc. Trans. 2000, 28, 557-563). Four members of the IRAK family have been identified: IRAK1, IRAK2, IRAK3, and IRAK4. These proteins are characterized by a typical N-terminal death domain that mediates interaction with MyD88-family adaptor proteins and a centrally located kinase domain. Of the four members in the mammalian IRAK family, TRAK2 and IRAK3 are thought to be catalytically inactive pseudokinases (Wesche et al., J. Biol. Chem. 1999, 274, 19403-19410), but the detailed roles of the two kinases are still largely unknown (Lagne et al., Structure 2021, 29, 238-251). Nonetheless, reports indicate the association of IRAK3 with negative regulation of TLR (toll-like receptor) signaling which is involved in detecting microorganisms and protecting multicellular organisms from infection (Kobayashi et al., Cell 2002, 110, 191-202). More recent studies have revealed the linkage between mutation or high expression levels of IRAK3 and various diseases such as asthma and cancer (Balaci et al., Am. J. Hum. Genet. 2007, 80 (6), 1103-1114; Kesselring, R. Cancer Cell 2016, 29 (5), 685-696), which suggests the potential of RAK3 as a drug target and the need for TRAK3 binding small molecules.

Protein degradation is a highly regulated and essential process that maintains cellular homeostasis. Selective identification and removal of damaged, misfolded, or excess proteins is achieved through the ubiquitin-proteasome pathway (UPP). The UPP is central to the regulation of almost all cellular processes. Ubiquitination of the protein is accomplished by an E3 ubiquitin ligase that binds to a protein and adds ubiquitin molecules to the protein, thus marking the protein for proteasome degradation.

Harnessing the UPP for therapeutic use has received significant interest (Zhou et al., Mol. Cell 2000, 6, 751-756). One promising therapy uses proteolysis targeting chimeras, commonly referred to as PROTACs, to effect removal of unwanted proteins by protein degradation (Scheepstra et al., Comp. Struct. Biotech. J. 2019, 17, 160-176). PROTACS are ligand directed degraders that bring together an E3 ligase and a target protein that is to be degraded. These bivalent molecules usually consist of an E3 ligase ligand connected through a linker moiety to small molecule that binds to the target protein. A PROTAC positions the E3 ligase at the appropriate distance and orientation to the target protein, allowing the latter to be ubiquitinated. The ubiquitinated target protein is subsequently recognized by the proteasome, where it is degraded.

Accordingly, in one aspect, provided herein are compounds that target IRAK3 for degradation.

### SUMMARY

Described herein, in certain embodiments, are compounds and compositions thereof for degrading IRAK3. In various embodiments, the compounds and compositions thereof may be used for treatment of cancer.

The present embodiments can be understood more fully by reference to the detailed description and examples, which are intended to exemplify non-limiting embodiments.

Embodiment A1 is a compound of Formula (I):

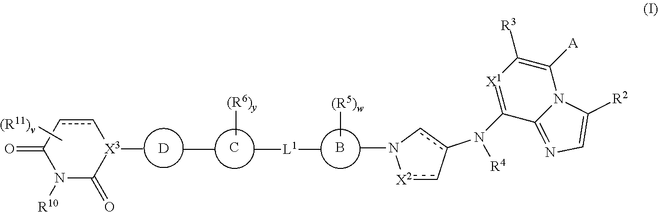

or a pharmaceutically acceptable salt thereof, wherein:
A is C₁-C₆ alkyl, phenyl, C₃-C₆ cycloalkyl, 5- to 6-membered heteroaryl, or 6- to 10-membered heterocyclyl, wherein the phenyl, cycloalkyl, heteroaryl, and heterocyclyl are substituted by x R¹ groups, and wherein the heteroaryl and heterocyclyl contain 1-3 heteroatoms selected from N and O;
each R¹ is independently halo, C₁-C₆ alkyl, C₃-C₆ cycloalkyl, C₁-C₆ alkoxy, C₁-C₆ haloalkyl, or —SO₂(C₁-C₆ alkyl);
or two R¹ groups on adjacent carbon atoms are taken together to form a fused C₃-C₆ cycloalkyl or a fused

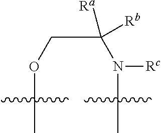

group;
R^{a} and R^{b} are each H or are taken together to form an oxo group;
R^{c} is H or C₁-C₆ alkyl;
x is 0-5;
R² is H or C₁-C₆ alkyl;
R³ is H or C₁-C₆ alkyl;
R⁴ is H or C₁-C₆ alkyl;
X¹ is CH or N;
X² is N or CH₂;
Ring B is C₃-C₆ cycloalkylene or 5- to 7-membered heterocyclylene containing 1 or 2 nitrogen atoms;
each R⁵ is independently halo, C₁-C₆ alkyl, or C₁-C₆ haloalkyl;
w is 0-5;
L¹ is —C(O)(CH₂)ₙ—, —(CH₂)ₙ—, or —(CH₂)ₙC(O)—;
n is 1-6;
Ring C is 5- to 10-membered heterocyclylene containing 1 or 2 nitrogen atoms;
each R⁶ is independently halo, C₁-C₆ haloalkyl, or C₁-C₆ alkyl;
y is 0-5;
Ring D is

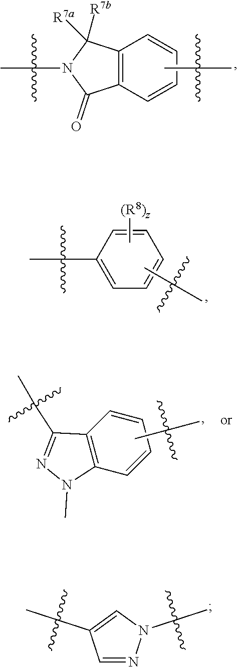

R^{7a} and R^{7b} are each H or are taken together to form an oxo group;
each R⁸ is independently halo, C₁-C₆ alkyl, C₁-C₆ haloalkyl, or C₁-C₆ alkoxy;
z is 0-4;
X³ is N or CR⁹;
R⁹ is H or C₁-C₆ alkyl;
R¹⁰ is H or C₁-C₆ alkyl;
each R¹¹ is independently halo, C₁-C₆ alkyl, or C₁-C₆ haloalkyl;
v is 0-4; and
each [symbol] is independently a single bond or double bond.

Embodiment A2 is the compound of embodiment A1, or a pharmaceutically acceptable salt thereof, wherein:
A is C₁-C₃ alkyl, phenyl, C₃-C₅ cycloalkyl, 5- to 6-membered heteroaryl, or 8- to 10-membered heterocyclyl, wherein the phenyl, cycloalkyl, heteroaryl, and heterocyclyl are substituted by x R¹ groups;
x is 0-3; and
each R¹ is independently halo, C₁-C₃ alkyl, C₃-C₅ cycloalkyl, C₁-C₃ alkoxy, C₁-C₃ haloalkyl, or —SO₂(C₁-C₃ alkyl);
or two R¹ groups on adjacent carbon atoms are taken together to form a fused C₃-C₅ cycloalkyl or a fused

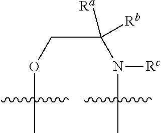

group;
R^{a} and R^{b} are each H or are taken together to form an oxo group; and
R^{c} is H or C₁-C₃ alkyl.

Embodiment A3 is the compound of embodiment A1 or A2, or a pharmaceutically acceptable salt thereof, wherein:
A is

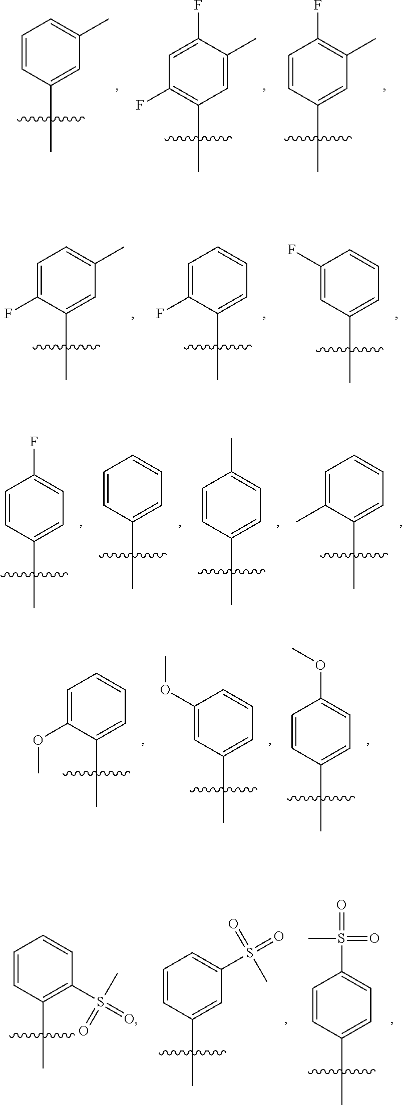

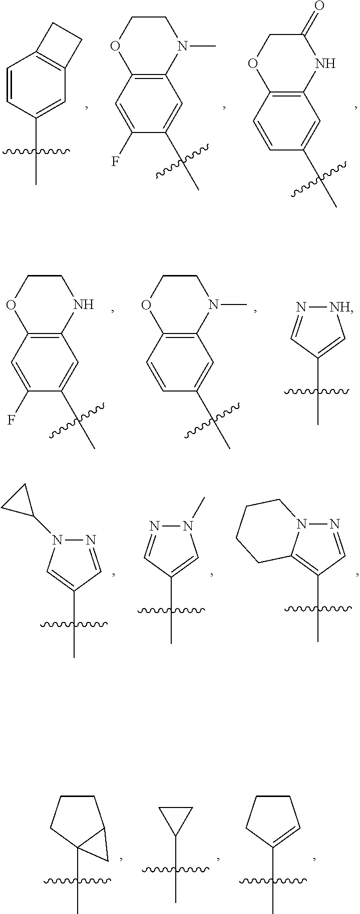

—CH₃, or —CH₂CH₃

Embodiment A4 is the compound of any one of embodiments A1-A3, or a pharmaceutically acceptable salt thereof, wherein:
R² is H or C₁-C₃ alkyl;
R³ is H or C₁-C₃ alkyl; and
R⁴ is H or C₁-C₃ alkyl.

Embodiment A5 is the compound of any one of embodiments A1-A4, or a pharmaceutically acceptable salt thereof, wherein:
X¹ is N.

Embodiment A6 is the compound of any one of embodiments A1-A5, or a pharmaceutically acceptable salt thereof, wherein:

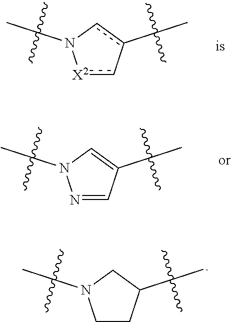

Embodiment A7 is the compound of any one of embodiments A1-A6, or a pharmaceutically acceptable salt thereof, wherein:
Ring B is C₄-C₆ cycloalkylene or 6- to 7-membered heterocyclylene containing one nitrogen atom;
w is 0-2; and
each R⁵ is independently halo, C₁-C₃ alkyl, or C₁-C₃ haloalkyl.

Embodiment A8 is the compound of any one of embodiments A1-A7, or a pharmaceutically acceptable salt thereof, wherein:

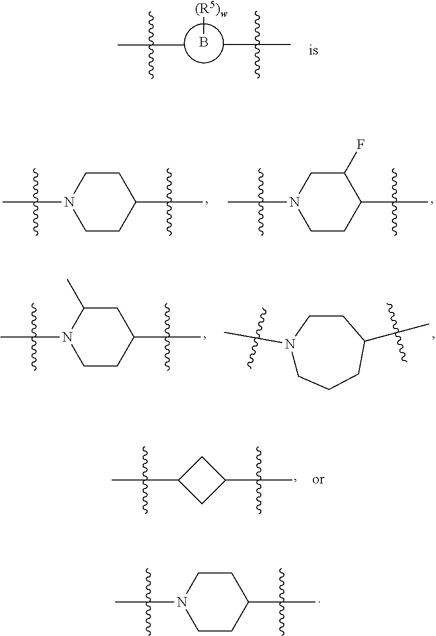

Embodiment A9 is the compound of any one of embodiments A1-A8, or a pharmaceutically acceptable salt thereof, wherein:
L¹ is —C(O)CH₂—, —(CH₂)ₙ—, or —CH₂C(O)—; and
n is 1-5.

Embodiment A10 is the compound of any one of embodiments A1-A9, or a pharmaceutically acceptable salt thereof, wherein:
Ring C is 6- to 8-membered heterocyclylene containing 1 or 2 nitrogen atoms;
y is 0-3; and
each R⁶ is independently halo, C₁-C₃ haloalkyl, or C₁-C₃ alkyl.

Embodiment A11 is the compound of any one of embodiments A1-A10, or a pharmaceutically acceptable salt thereof, wherein:

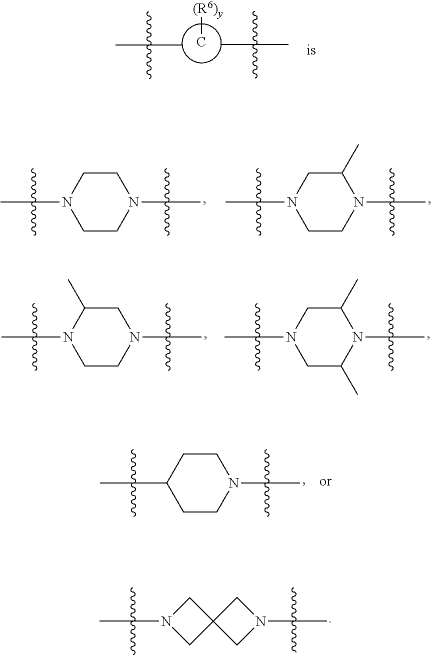

Embodiment A12 is the compound of any one of embodiments A1-A11, or a pharmaceutically acceptable salt thereof, wherein:
Ring D is

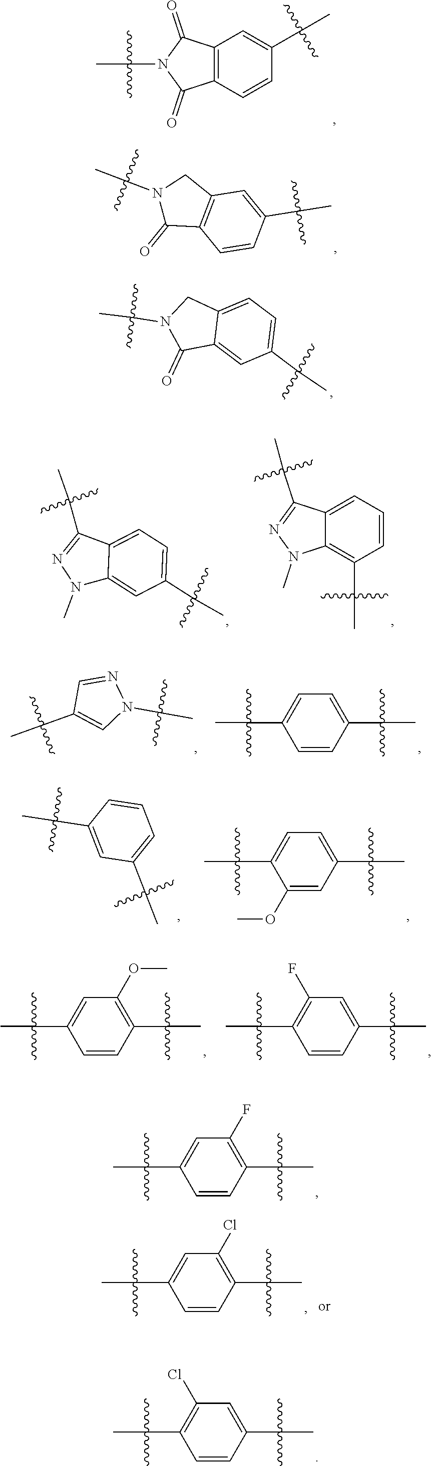

Embodiment A13 is the compound of any one of embodiments A1-A12, or a pharmaceutically acceptable salt thereof, wherein:
X³ is CR⁹;
R⁹ is H or C₁-C₃ alkyl;
R¹⁰ is H or C₁-C₃ alkyl;
v is 0-2; and
each R¹¹ is independently halo, C₁-C₃ alkyl, or C₁-C₃ haloalkyl.

Embodiment A14 is the compound of any one of embodiments A1-A13, or a pharmaceutically acceptable salt thereof, wherein:

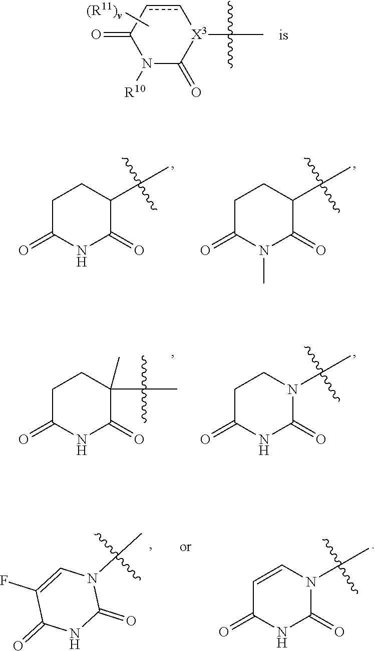

Embodiment A15 is the compound of any one of embodiments A1-A14, or a pharmaceutically acceptable salt thereof, wherein the compound is of Formula (IIIa), (IIIb), or (IIIc):

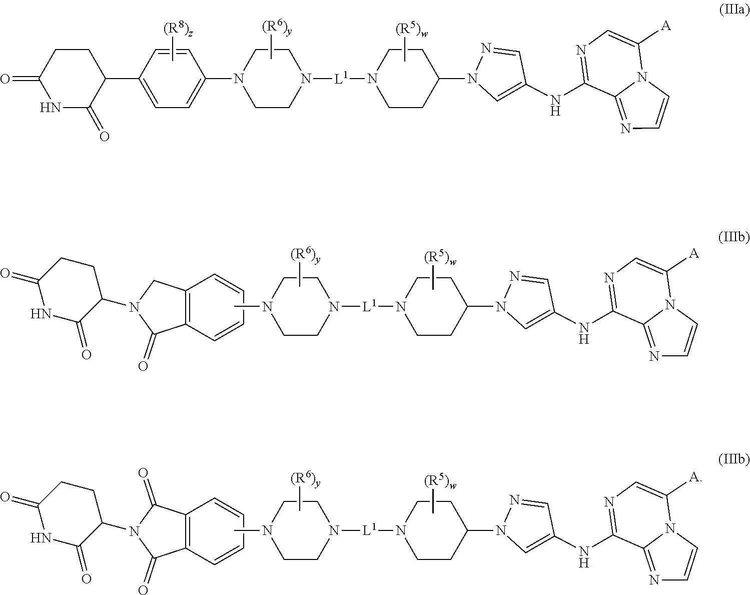

Embodiment A16 is the compound of any one of embodiments A1-A15, or a pharmaceutically acceptable salt thereof, wherein the compound is of Formula (IVa) or (IVb):

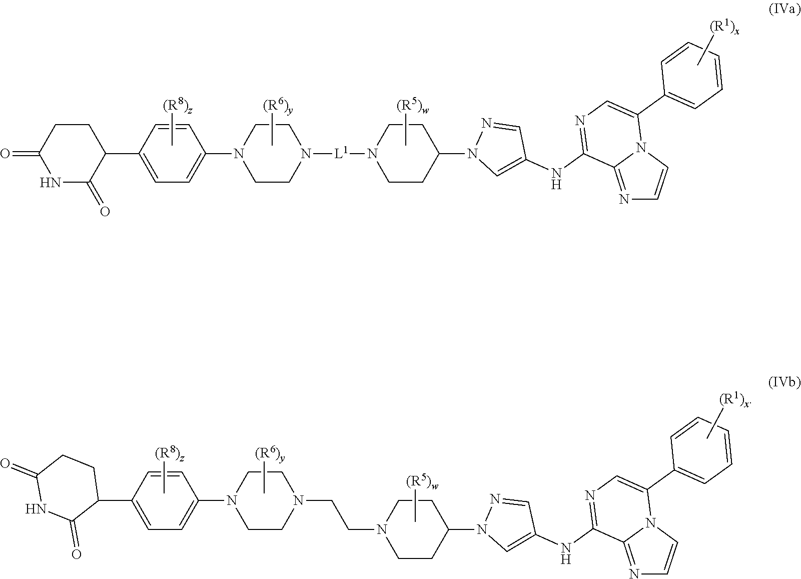

Embodiment A17 is a compound selected from the compounds of Table 1 or a pharmaceutically acceptable salt thereof.

Embodiment A18 is a pharmaceutical composition comprising the compound of any one of embodiments A1-A17, or a pharmaceutically acceptable salt thereof, and a pharmaceutically acceptable excipient.

Embodiment A19 is a method of modulating Interleukin-1 Receptor-Associated Kinase 3 (IRAK3) comprising contacting IRAK3 with an effective amount of the compound of any one of embodiments A1-A17, or a pharmaceutically acceptable salt thereof, or the pharmaceutical composition of embodiment A18.

Embodiment A20 is a method of (i) treating cancer in a subject in need thereof, comprising administering to the subject an effective amount of the compound of any one of embodiments A1-A17, or a pharmaceutically acceptable salt thereof, or the pharmaceutical composition of embodiment A18, optionally wherein the cancer is selected from bladder cancer, breast cancer, esophgeal cancer, colon cancer, head and neck cancer, kidney cancer, lung cancer, pancreatic cancer, prostate cancer, melanoma, and gastric cancer; or (ii) enhancing immunity in a subject receiving a vaccine, comprising administering to the subject an effective amount of the compound of any one of embodiments A1-A17, or a pharmaceutically acceptable salt thereof, or the pharmaceutical composition of embodiment A18.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “cycloalkylene” group refers to a divalent “cycloalkyl” group.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing at least one heteroatom. The phrase further includes spiro polycyclic ring systems containing at least one heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heterocyclylene” group refers to a divalent “heterocyclyl” group.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

An “alkoxy” group is —O-(alkyl), wherein alkyl is defined above.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of Formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

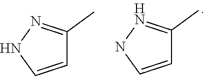

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (2H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an IRAK3 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

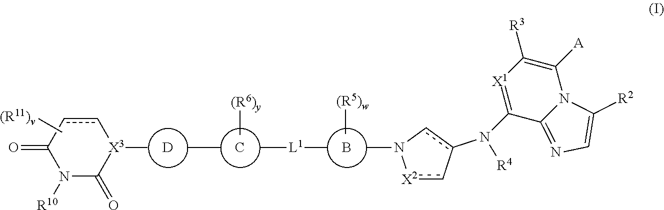

or a pharmaceutically acceptable salt thereof, wherein:
A is C₁-C₆ alkyl, phenyl, C₃-C₆ cycloalkyl, 5- to 6-membered heteroaryl, or 6- to 10-membered heterocyclyl, wherein the phenyl, cycloalkyl, heteroaryl, and heterocyclyl are substituted by x R¹ groups, and wherein the heteroaryl and heterocyclyl contain 1-3 heteroatoms selected from N and O;
each R¹ is independently halo, C₁-C₆ alkyl, C₃-C₆ cycloalkyl, C₁-C₆ alkoxy, C₁-C₆ haloalkyl, or —SO₂(C₁-C₆ alkyl);
or two R¹ groups on adjacent carbon atoms are taken together to form a fused C₃-C₆ cycloalkyl or a fused

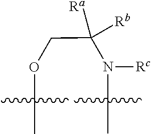

group;
R^{a} and R^{b} are each H or are taken together to form an oxo group;
R^{c} is H or C₁-C₆ alkyl;
x is 0-5;
R² is H or C₁-C₆ alkyl;
R³ is H or C₁-C₆ alkyl;
R⁴ is H or C₁-C₆ alkyl;
X¹ is CH orN;
X² is N or CH₂;
Ring B is C₃-C₆ cycloalkylene or 5- to 7-membered heterocyclylene containing 1 or 2 nitrogen atoms;
each R⁵ is independently halo, C₁-C₆ alkyl, or C₁-C₆ haloalkyl;
w is 0-5;
L¹ is —C(O)(CH₂)ₙ—, —(CH₂)ₙ—, or —(CH₂)ₙC(O)—;
n is 1-6;
Ring C is 5- to 10-membered heterocyclylene containing 1 or 2 nitrogen atoms;
each R⁶ is independently halo, C₁-C₆ haloalkyl, or C₁-C₆ alkyl;
y is 0-5;
Ring D is

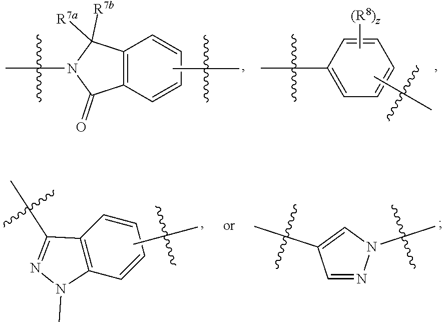

R^{7a} and R^{7b} are each H or are taken together to form an oxo group;
each R⁸ is independently halo, C₁-C₆ alkyl, C₁-C₆ haloalkyl, or C₁-C₆ alkoxy;
z is 0-4;
X³ is N or CR⁹;
R⁹ is H or C₁-C₆ alkyl;
R¹⁰ is H or C₁-C₆ alkyl;
each R¹¹ is independently halo, C₁-C₆ alkyl, or C₁-C₆ haloalkyl;
v is 0-4; and
each [symbol] is independently a single bond or double bond.

In some embodiments, A is C₁-C₆ alkyl, phenyl, C₃-C₆ cycloalkyl, 5- to 6-membered heteroaryl, or 6- to 10-membered heterocyclyl, wherein the phenyl, cycloalkyl, heteroaryl, and heterocyclyl are substituted by x R¹ groups, and wherein the heteroaryl and heterocyclyl contain 1-3 heteroatoms selected from N and O. In some embodiments, A is C₁-C₃ alkyl, phenyl, C₃-C₅ cycloalkyl, 5- to 6-membered heteroaryl, or 9- to 10-membered heterocyclyl, wherein the phenyl, cycloalkyl, heteroaryl, and heterocyclyl are substituted by x R¹ groups.

In some embodiments, A is C₁-C₆ alkyl. In some embodiments, A is C₁-C₃ alkyl. In some embodiments, A is methyl, ethyl, n-propyl, or isopropyl. In some embodiments, A is methyl. In some embodiments, A is ethyl. In some embodiments, A is n-propyl. In some embodiments, A is isopropyl. In some embodiments, A is methyl or ethyl.

In some embodiments, A is phenyl substituted by x R¹ groups.

In some embodiments, A is C₃-C₆ cycloalkyl substituted by x R¹ groups. In some embodiments, A is C₃-C₅ cycloalkyl. In some embodiments, A is cyclopropyl, cyclobutyl, cyclopentyl, or cyclohexyl. In some embodiments, R⁰ is cyclopropyl. In some embodiments, A is saturated C₃-C₆ cycloalkyl. In some embodiments, A is partially unsaturated C₃-C₆ cycloalkyl. In some embodiments, A is cyclopropylene, cyclobutylene, cyclopentylene, or cyclohexylene. In some embodiments, A is cyclopentylene or cyclohexylene. In some embodiments, A is cyclopentylene. In some embodiments, A is a fused bicyclic C₄-C₆ cycloalkyl. In some embodiments, A is a fused bicyclic C₄ cycloalkyl. In some embodiments, A is a fused bicyclic C₅ cycloalkyl. In some embodiments, A is a fused bicyclic C₆ cycloalkyl. In some embodiments, A is a cyclopropyl fused to a cyclobutyl or cyclopentyl ring. In some embodiments, A is a cyclopropyl fused to a cyclopentyl ring. In any of these variations, the cycloalkyl is substituted by x R¹ groups.

In some embodiments, A is 5- to 6-membered heteroaryl containing 1-3 heteroatoms selected from N and O and substituted by x R¹ groups. In some embodiments, A is 5-membered heteroaryl containing 1-3 heteroatoms selected from N and O. In some embodiments, A is 6-membered heteroaryl containing 1-3 heteroatoms selected from N and O. In some embodiments, the heteroaryl contains 1-2 heteroatoms selected from N and O. In some embodiments, the heteroaryl contains 1 heteroatom selected from N and O. In some embodiments, the heteroaryl contains one oxygen atom. In some embodiments, the heteroaryl contains one nitrogen atom. In some embodiments, the heteroaryl contains 2 heteroatoms selected from N and O. In some embodiments, the heteroaryl contains one nitrogen atom and one oxygen atom. In some embodiments, the heteroaryl contains two nitrogen atoms. In some embodiments, A is pyridinyl, pyrazinyl, pyrimidinyl, pyridazinyl, triazinyl, pyrrolyl, imidazolyl, pyrazolyl, triazolyl, oxazolyl, or isoxazolyl. In some embodiments, A is pyrazolyl. In any of these variations, the heteroaryl is substituted by x R¹ groups.

In some embodiments, A is 6- to 10-membered heterocyclyl containing 1-3 heteroatoms selected from N and O and substituted by x R¹ groups. In some embodiments, A is 8- to 10-membered heterocyclyl containing 1-3 heteroatoms selected from N and O and substituted by x R¹ groups. In some embodiments, A is 8- to 9-membered heterocyclyl containing 1-3 heteroatoms selected from N and O and substituted by x R¹ groups. In some embodiments, A is 9- to 10-membered heterocyclyl containing 1-3 heteroatoms selected from N and O and substituted by x R¹ groups. In some embodiments, A is 8-membered heterocyclyl containing 1-3 heteroatoms selected from N and O and substituted by x R¹ groups. In some embodiments, A is 9-membered heterocyclyl containing 1-3 heteroatoms selected from N and O and substituted by x R¹ groups. In some embodiments, A is 10-membered heterocyclyl containing 1-3 heteroatoms selected from N and O and substituted by x R¹ groups. In some embodiments, the heterocyclyl is a monocyclic heterocyclyl. In some embodiments, the heterocyclyl is a fused bicyclic heterocyclyl. In some embodiments, the fused bicyclic heterocyclyl contains a heterocyclyl fused to an aryl group. In some embodiments, the fused bicyclic heterocyclyl contains a heterocyclyl fused to a heteroaryl group. In any of these variations, the heterocyclyl is substituted by x R¹ groups.

In some embodiments, x is 0-5. In some embodiments, x is 0-3. In some embodiments, x is 0. In some embodiments, x is 1. In some embodiments, x is 2. In some embodiments, x is 3. In some embodiments, x is 4. In some embodiments, x is 5.

In some embodiments, each R¹ is independently halo, C₁-C₆ alkyl, C₃-C₆ cycloalkyl, C₁-C₆ alkoxy, C₁-C₆ haloalkyl, or —SO₂(C₁-C₆ alkyl). In some embodiments, each R¹ is independently halo, C₁-C₃ alkyl, C₃-C₅ cycloalkyl, C₁-C₃ alkoxy, C₁-C₃ haloalkyl, or —SO₂(C₁-C₃ alkyl). In some embodiments, each R¹ is independently F, —CH₃, cyclopropyl, —OCH₃, or —SO₂(CH₃).

In some embodiments, R¹ is halo. In some embodiments, R¹ is Cl, F, or Br. In some embodiments, R¹ is Cl. In some embodiments, R¹ is F. In some embodiments, R¹ is Br.

In some embodiments, R¹ is C₁-C₆ alkyl. In some embodiments, R¹ is C₁-C₃ alkyl. In some embodiments, R¹ is methyl, ethyl, n-propyl, or isopropyl. In some embodiments, R¹ is methyl. In some embodiments, R¹ is ethyl. In some embodiments, R¹ is n-propyl. In some embodiments, R¹ is isopropyl.

In some embodiments, R¹ is C₃-C₆ cycloalkyl. In some embodiments, R¹ is C₃-C₅ cycloalkyl. In some embodiments, R¹ is cyclopropyl, cyclobutyl, cyclopentyl, or cyclohexyl. In some embodiments, R¹ is cyclopropyl.

In some embodiments, R¹ is C₁-C₆ alkoxy. In some embodiments, R¹ is C₁-C₃ alkoxy. In some embodiments, R¹ is —OCH₃, —OCH₂CH₃, —OCH₂CH₂CH₃, or —OCH(CH₃)₂. In some embodiments, R¹ is —OCH₃. In some embodiments, R¹ is —OCH₂CH₃.

In some embodiments, R¹ is C₁-C₆ haloalkyl. In some embodiments, R¹ is C₁-C₆ haloalkyl containing 1-13 halogen atoms. In some embodiments, R¹ is C₁-C₃ haloalkyl. In some embodiments, R¹ is C₁-C₃ haloalkyl containing 1-7 halogen atoms. In some embodiments, R¹ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R¹ is —CF₃. In some embodiments, R¹ is —CHF₂.

In some embodiments, R¹ is —SO₂(C₁-C₆ alkyl). In some embodiments, R¹ is —SO₂(C₁-C₃ alkyl). In some embodiments, R¹ is —SO₂(CH₃), —SO₂(CH₂CH₃), or —SO₂(CH₂CH₂CH₃). In some embodiments, R¹ is —SO₂(CH₃).

In some embodiments, two R¹ groups on adjacent carbon atoms are taken together to form a fused C₃-C₆ cycloalkyl or a fused

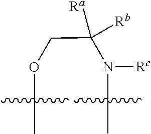

group, wherein R^{a} and R^{b} are each H or are taken together to form an oxo group, and R^{c} is H or C₁-C₆ alkyl. In some embodiments, two R¹ groups on adjacent carbon atoms are taken together to form a fused C₃-C₅ cycloalkyl or a fused

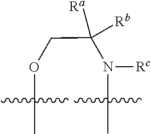

group, wherein R^{a} and R^{b} are each H or are taken together to form an oxo group, and R^{c} is H or C₁-C₃ alkyl. In some embodiments, two R¹ groups on adjacent carbon atoms are taken together to form a fused cyclopropyl, cyclobutyl,

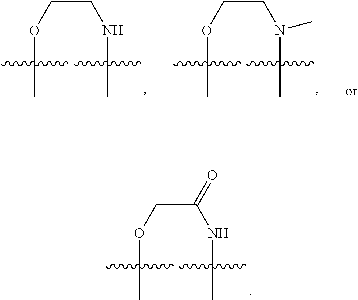

In some embodiments, two R¹ groups on adjacent carbon atoms are taken together to form a fused C₃-C₆ cycloalkyl. In some embodiments, two R¹ groups on adjacent carbon atoms are taken together to form a fused C₃-C₅ cycloalkyl. In some embodiments, two R¹ groups on adjacent carbon atoms are taken together to form a fused cyclopropyl, cyclobutyl, or cyclopentyl. In some embodiments, two R¹ groups on adjacent carbon atoms are taken together to form a fused cyclopropyl or cyclobutyl. In some embodiments, two R¹ groups on adjacent carbon atoms are taken together to form a fused cyclopropyl. In some embodiments, two R¹ groups on adjacent carbon atoms are taken together to form a fused cyclobutyl.

In some embodiments, two R¹ groups on adjacent carbon atoms are taken together to form a fused

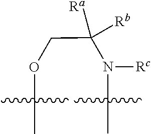

group, wherein R^{a} and R^{b} are each H or are taken together to form an oxo group, and R^{c} is H or C₁-C₆ alkyl. In some embodiments, two R¹ groups on adjacent carbon atoms are taken together to form a fused

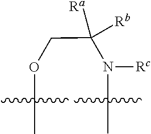

group, wherein R^{a} and R^{b} are each H or are taken together to form an oxo group, and R^{c} is H or C₁-C₃ alkyl. In some embodiments, R^{a} and R^{b} are each H. In some embodiments, R^{a} and R^{b} are taken together to form an oxo group. In some embodiments, R^{c} is H. In some embodiments, R^{c} is C₁-C₆ alkyl, such as C₁-C₃ alkyl. In some embodiments, R^{c} is methyl, ethyl, n-propyl, or isopropyl. In some embodiments, R^{c} is methyl or ethyl. In some embodiments, two R¹ groups on adjacent carbon atoms are taken together to form a fused

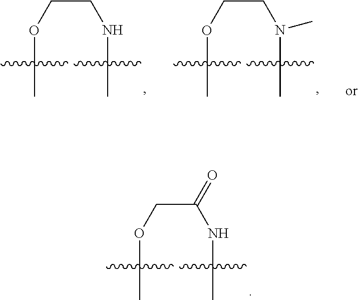

In some embodiments, A is:

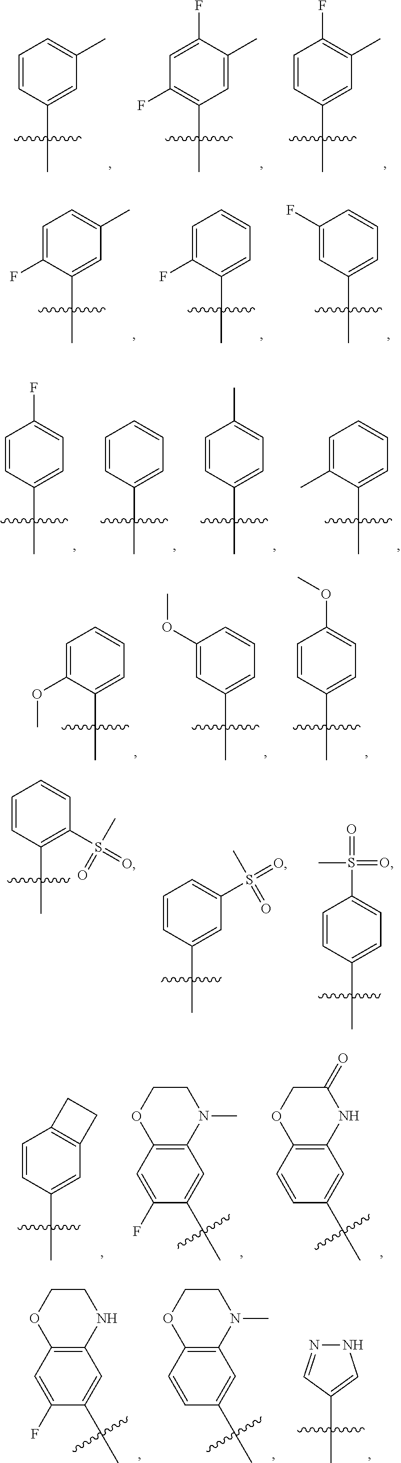

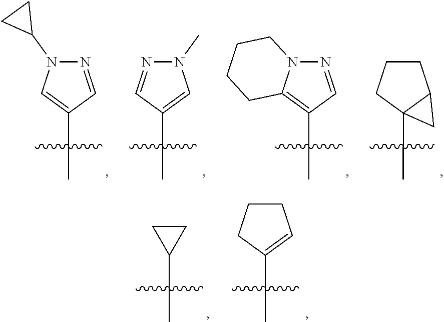

—CH₃, or —CH₂CH₃.

In some embodiments, R² is H or C₁-C₆ alkyl. In some embodiments, R² is H or C₁-C₃ alkyl. In some embodiments, R² is H or —CH₃.

In some embodiments, R² is H.

In some embodiments, R² is C₁-C₆ alkyl. In some embodiments, R² is C₁-C₃ alkyl. In some embodiments, R² is methyl, ethyl, n-propyl, or isopropyl. In some embodiments, R² is methyl. In some embodiments, R² is ethyl. In some embodiments, R² is n-propyl. In some embodiments, R² is isopropyl. In some embodiments, R² is methyl or ethyl.

In some embodiments, R³ is H or C₁-C₆ alkyl. In some embodiments, R³ is H or C₁-C₃ alkyl. In some embodiments, R³ is H or —CH₃.

In some embodiments, R³ is H.

In some embodiments, R³ is C₁-C₆ alkyl. In some embodiments, R³ is C₁-C₃ alkyl. In some embodiments, R³ is methyl, ethyl, n-propyl, or isopropyl. In some embodiments, R³ is methyl. In some embodiments, R³ is ethyl. In some embodiments, R³ is n-propyl. In some embodiments, R³ is isopropyl. In some embodiments, R³ is methyl or ethyl.

In some embodiments, R⁴ is H or C₁-C₆ alkyl. In some embodiments, R⁴ is H or C₁-C₃ alkyl. In some embodiments, R⁴ is H or —CH₃.

In some embodiments, R⁴ is H.

In some embodiments, R⁴ is C₁-C₆ alkyl. In some embodiments, R⁴ is C₁-C₃ alkyl. In some embodiments, R⁴ is methyl, ethyl, n-propyl, or isopropyl. In some embodiments, R⁴ is methyl. In some embodiments, R⁴ is ethyl. In some embodiments, R⁴ is n-propyl. In some embodiments, R⁴ is isopropyl. In some embodiments, R⁴ is methyl or ethyl.

In some embodiments, X¹ is CH or N. In some embodiments, X¹ is CH. In some embodiments, X¹ is N.

In some embodiments, X² is CH₂ or N. In some embodiments, X² is CH₂. In some embodiments, X² is N.

In some embodiments, each [symbol] is independently a single bond or double bond. In some embodiments, [symbol] is a single bond. In some embodiments, [symbol] is a double bond.

In some embodiments,

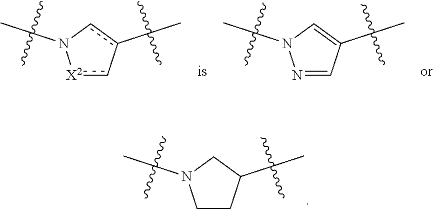

In some embodiments, Ring B is C₃-C₆ cycloalkylene or 5- to 7-membered heterocyclylene containing 1 or 2 nitrogen atoms. In some embodiments, Ring B is C₄-C₆ cycloalkylene or 6- to 7-membered heterocyclylene containing one nitrogen atom.

In some embodiments, Ring B is C₃-C₆ cycloalkylene. In some embodiments, Ring B is C₄ cycloalkylene. In some embodiments, Ring B is C₅ cycloalkylene. In some embodiments, Ring B is C₆ cycloalkylene. In some embodiments, Ring B is cyclobutylene, cyclopentylene, or cyclohexylene. In some embodiments, Ring B is

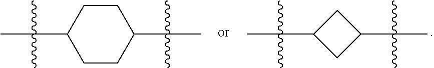 [SMILES: *C1CCC(*)CC1 ; *C1CC(*)C1]

In some embodiments, Ring B is 5- to 7-membered heterocyclylene containing 1 or 2 nitrogen atoms. In some embodiments, Ring B is 5-membered heterocyclylene containing 1 or 2 nitrogen atoms. In some embodiments, Ring B is 6-membered heterocyclylene containing 1 or 2 nitrogen atoms. In some embodiments, Ring B is 7-membered heterocyclylene containing 1 or 2 nitrogen atoms. In some embodiments, Ring B is 6- to 7-membered heterocyclylene containing one nitrogen atom. In some embodiments, Ring B is 6- to 7-membered heterocyclylene containing two nitrogen atoms. In some embodiments, Ring B is

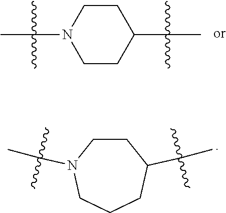 [SMILES: *C1CCN(*)CC1 ; *C1CCCN(*)CC1]

In some embodiments, Ring B is:

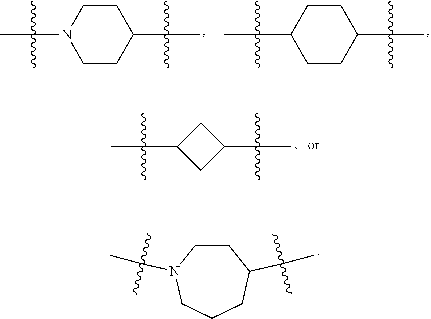 [SMILES: *C1CCN(*)CC1 ; *C1CCC(*)CC1 ; *C1CC(*)C1 ; *C1CCCN(*)CC1]

In some embodiments, w is 0-5. In some embodiments, w is 0-2. In some embodiments, w is 0. In some embodiments, w is 1. In some embodiments, w is 2. In some embodiments, w is 3. In some embodiments, w is 4. In some embodiments, w is 5.

In some embodiments, each R⁵ is independently halo, C₁-C₆ alkyl, or C₁-C₆ haloalkyl. In some embodiments, each R⁵ is independently halo, C₁-C₃ alkyl, or C₁-C₃ haloalkyl. In some embodiments, each R⁵ is independently F, —CF₃, or —CH₃.

In some embodiments, R⁵ is halo. In some embodiments, R⁵ is Cl, F, or Br. In some embodiments, R⁵ is Cl. In some embodiments, R⁵ is F. In some embodiments, R⁵ is Br.

In some embodiments, R⁵ is C₁-C₆ alkyl. In some embodiments, R⁵ is C₁-C₃ alkyl. In some embodiments, R⁵ is methyl, ethyl, n-propyl, or isopropyl. In some embodiments, R⁵ is methyl. In some embodiments, R⁵ is ethyl. In some embodiments, R⁵ is n-propyl. In some embodiments, R⁵ is isopropyl.

In some embodiments, R⁵ is C₁-C₆ haloalkyl. In some embodiments, R⁵ is C₁-C₆ haloalkyl containing 1-13 halogen atoms. In some embodiments, R⁵ is C₁-C₃ haloalkyl. In some embodiments, R⁵ is C₁-C₃ haloalkyl containing 1-7 halogen atoms. In some embodiments, R⁵ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R⁵ is —CF₃. In some embodiments, R⁵ is —CHF₂.

In some embodiments,

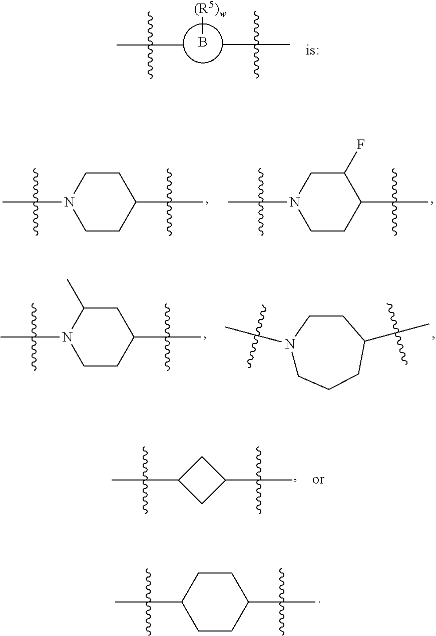

In some embodiments, L¹ is —C(O)(CH₂)ₙ—, —(CH₂)ₙ—, or —(CH₂)ₙC(O)—. In some embodiments, L¹ is —C(O)CH₂—, —(CH₂)ₙ—, or —CH₂C(O)—, wherein n is 1-5. In some embodiments, L¹ is —C(O)CH₂—, —CH₂—, —CH₂CH₂—, —CH₂CH₂CH₂—, —CH₂CH₂CH₂CH₂—, or —CH₂C(O)—.

In some embodiments, L¹ is —C(O)(CH₂)ₙ—, wherein n is 1-6. In some embodiments, L¹ is —C(O)(CH₂)—. In some embodiments, L¹ is —C(O)(CH₂)₂—. In some embodiments, L¹ is —C(O)(CH₂)₃—. In some embodiments, L¹ is —C(O)(CH₂)₄—. In some embodiments, L¹ is —C(O)(CH₂)₅—. In some embodiments, L¹ is —C(O)(CH₂)₆—.

In some embodiments, L¹ is —(CH₂)ₙ—, wherein n is 1-6. In some embodiments, L¹ is —(CH₂)—. In some embodiments, L¹ is —(CH₂)₂—. In some embodiments, L¹ is —(CH₂)₃—. In some embodiments, L¹ is —(CH₂)₄—. In some embodiments, L¹ is —(CH₂)₅—. In some embodiments, L¹ is —(CH₂)₆—.

In some embodiments, L¹ is —(CH₂)ₙC(O)—, wherein n is 1-6. In some embodiments, L¹ is —(CH₂)C(O)—. In some embodiments, L¹ is —(CH₂)₂C(O)—. In some embodiments, L¹ is —(CH₂)₃C(O)—. In some embodiments, L¹ is —(CH₂)₄C(O)—. In some embodiments, L¹ is —(CH₂)₅C(O)—. In some embodiments, L¹ is —(CH₂)₆C(O)—.

In some embodiments, Ring C is 5- to 10-membered heterocyclylene containing 1 or 2 nitrogen atoms. In some embodiments, Ring C is 6- to 8-membered heterocyclylene containing 1 or 2 nitrogen atoms. In some embodiments, the heterocyclene contains one nitrogen atom. In some embodiments, the heterocyclene contains two nitrogen atoms. In some embodiments, the heterocyclylene is monocyclic. In some embodiments, the heterocyclylene is piperazinylene, piperidinylene, or pyrrolidinylene. In some embodiments, the heterocyclylene is polycyclic. In some embodiments, the heterocyclylene is spiro.

In some embodiments, Ring C is:

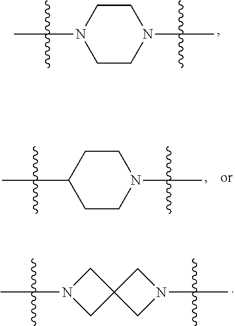 [SMILES: *N1CCN(*)CC1 ; *C1CCN(*)CC1 ; *N1CC2(C1)CN(*)C2]

In some embodiments, y is 0-5. In some embodiments, y is 0-3. In some embodiments, y is 0. In some embodiments, y is 1. In some embodiments, y is 2. In some embodiments, y is 3. In some embodiments, y is 4. In some embodiments, y is 5.

In some embodiments, each R⁶ is independently halo, C₁-C₆ haloalkyl, or C₁-C₆ alkyl. In some embodiments, each R⁶ is independently halo, C₁-C₃ haloalkyl, or C₁-C₃ alkyl. In some embodiments, each R⁶ is independently Cl, —CF₃, or —CH₃.

In some embodiments, R⁶ is halo. In some embodiments, R⁶ is Cl, F, or Br. In some embodiments, R⁶ is Cl. In some embodiments, R⁶ is F. In some embodiments, R⁶ is Br.

In some embodiments, R⁶ is C₁-C₆ haloalkyl. In some embodiments, R⁶ is C₁-C₆ haloalkyl containing 1-13 halogen atoms. In some embodiments, R⁶ is C₁-C₃ haloalkyl. In some embodiments, R⁶ is C₁-C₃ haloalkyl containing 1-7 halogen atoms. In some embodiments, R⁶ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R⁶ is —CF₃. In some embodiments, R⁶ is —CHF₂.

In some embodiments, R⁶ is C₁-C₆ alkyl. In some embodiments, R⁶ is C₁-C₃ alkyl. In some embodiments, R⁶ is methyl, ethyl, n-propyl, or isopropyl. In some embodiments, R⁶ is methyl. In some embodiments, R⁶ is ethyl. In some embodiments, R⁶ is n-propyl. In some embodiments, R⁶ is isopropyl.

In some embodiments,

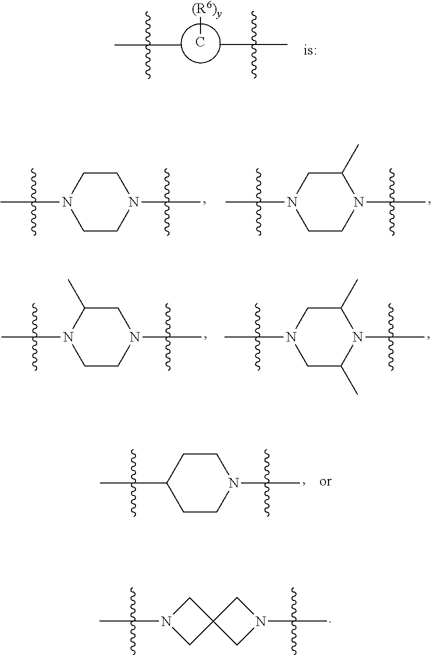

In some embodiments, Ring D is:

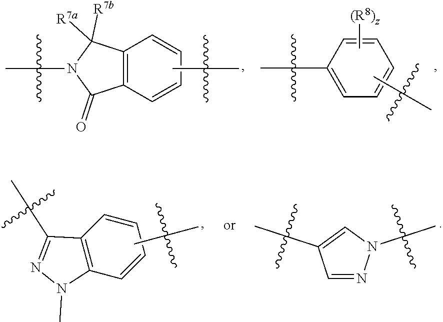

In some embodiments, Ring D is

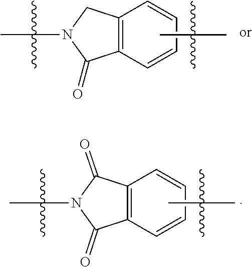

In some embodiments, Ring D is

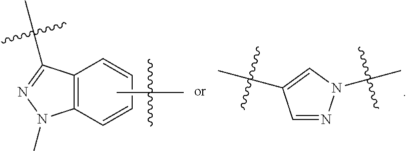

In some embodiments, Ring D is

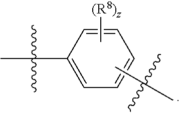

In some embodiments, Ring D is

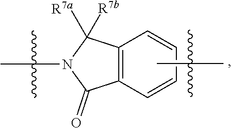

wherein R^{7a} and R^{7b} are each H or are taken together to form an oxo group. In some embodiments, Ring D is

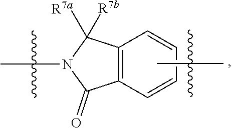

wherein R^{7a} and R^{7b} are each H. In some embodiments, Ring D is

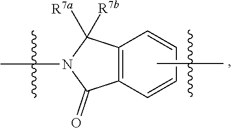

wherein R^{7a} and R^{7b} are taken together to form an oxo group. In some embodiments, Ring D is

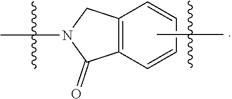

In some embodiments, Ring D is

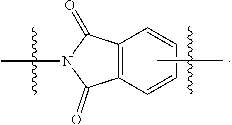

In some embodiments, Ring D is

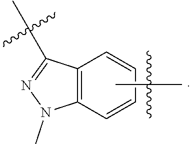

In some embodiments, Ring D is

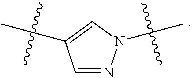 [SMILES: *c1cnn(*)c1]

In some embodiments, Ring D is

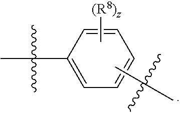

In some embodiments, z is 0-4. In some embodiments, z is 0-2. In some embodiments, z is 0. In some embodiments, z is 1. In some embodiments, z is 2. In some embodiments, z is 3. In some embodiments, z is 4.

In some embodiments, each R⁸ is independently halo, C₁-C₆ alkyl, C₁-C₆ haloalkyl, or C₁-C₆ alkoxy. In some embodiments, each R⁸ is independently halo, C₁-C₃ alkyl, C₁-C₃ haloalkyl, or C₁-C₃ alkoxy. In some embodiments, each R⁸ is independently F, Cl, —CH₃, —OCH₃, or —CF₃.

In some embodiments, R⁸ is halo. In some embodiments, R⁸ is Cl, F, or Br. In some embodiments, R⁸ is Cl. In some embodiments, R⁸ is F. In some embodiments, R⁸ is Br.

In some embodiments, R⁸ is C₁-C₆ alkyl. In some embodiments, R⁸ is C₁-C₃ alkyl. In some embodiments, R⁸ is methyl, ethyl, n-propyl, or isopropyl. In some embodiments, R⁸ is methyl. In some embodiments, R⁸ is ethyl. In some embodiments, R⁸ is n-propyl. In some embodiments, R⁸ is isopropyl.

In some embodiments, R⁸ is C₁-C₆ haloalkyl. In some embodiments, R⁸ is C₁-C₆ haloalkyl containing 1-13 halogen atoms. In some embodiments, R⁸ is C₁-C₃ haloalkyl. In some embodiments, R⁸ is C₁-C₃ haloalkyl containing 1-7 halogen atoms. In some embodiments, R⁸ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R⁸ is —CF₃. In some embodiments, R⁸ is —CHF₂.

In some embodiments, R⁸ is C₁-C₆ alkoxy. In some embodiments, R⁸ is C₁-C₃ alkoxy. In some embodiments, R⁸ is —OCH₃, —OCH₂CH₃, —OCH₂CH₂CH₃, or —OCH(CH₃)₂. In some embodiments, R⁸ is —OCH₃. In some embodiments, R⁸ is —OCH₂CH₃.

In some embodiments, Ring D is:

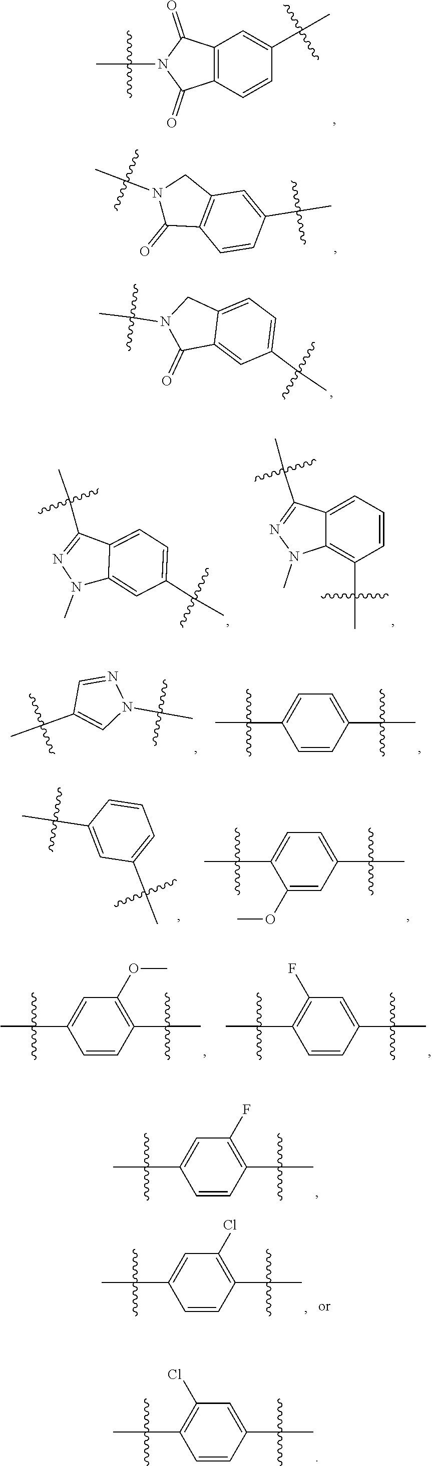

In some embodiments, X³ is N or CR⁹. In some embodiments, X³ is N. In some embodiments, X³ is CR⁹.

In some embodiments, R⁹ is H or C₁-C₆ alkyl. In some embodiments, R⁹ is H or C₁-C₃ alkyl. In some embodiments, R⁹ is H or —CH₃.

In some embodiments, R⁹ is H.

In some embodiments, R⁹ is C₁-C₆ alkyl. In some embodiments, R⁹ is C₁-C₃ alkyl. In some embodiments, R⁹ is methyl, ethyl, n-propyl, or isopropyl. In some embodiments, R⁹ is methyl. In some embodiments, R⁹ is ethyl. In some embodiments, R⁹ is n-propyl. In some embodiments, R⁹ is isopropyl.

In some embodiments, R¹⁰ is H or C₁-C₆ alkyl. In some embodiments, R¹⁰ is H or C₁-C₃ alkyl. In some embodiments, R¹⁰ is H or —CH₃.

In some embodiments, R¹⁰ is H.

In some embodiments, R¹⁰ is C₁-C₆ alkyl. In some embodiments, R¹⁰ is C₁-C₃ alkyl. In some embodiments, R¹⁰ is methyl, ethyl, n-propyl, or isopropyl. In some embodiments, R¹⁰ is methyl. In some embodiments, R¹⁰ is ethyl. In some embodiments, R¹⁰ is n-propyl. In some embodiments, R¹⁰ is isopropyl.

In some embodiments, v is 0-4. In some embodiments, v is 0-2. In some embodiments, v is 0. In some embodiments, v is 1. In some embodiments, v is 2. In some embodiments, v is 3. In some embodiments, v is 4.

In some embodiments, each R¹¹ is independently halo, C₁-C₆ alkyl, or C₁-C₆ haloalkyl. In some embodiments, each R¹¹ is independently halo, C₁-C₃ alkyl, or C₁-C₃ haloalkyl. In some embodiments, each R¹¹ is independently F, —CH₃, or —CF₃.

In some embodiments, R¹¹ is halo. In some embodiments, R¹¹ is Cl, F, or Br. In some embodiments, R¹¹ is Cl. In some embodiments, R¹¹ is F. In some embodiments, R¹¹ is Br.

In some embodiments, R¹¹ is C₁-C₆ alkyl. In some embodiments, R¹¹ is C₁-C₃ alkyl. In some embodiments, R¹¹ is methyl, ethyl, n-propyl, or isopropyl. In some embodiments, R¹¹ is methyl. In some embodiments, R¹¹ is ethyl. In some embodiments, R¹¹ is n-propyl. In some embodiments, R¹¹ is isopropyl.

In some embodiments, R¹¹ is C₁-C₆ haloalkyl. In some embodiments, R¹¹ is C₁-C₆ haloalkyl containing 1-13 halogen atoms. In some embodiments, R¹¹ is C₁-C₃ haloalkyl. In some embodiments, R¹¹ is C₁-C₃ haloalkyl containing 1-7 halogen atoms. In some embodiments, R¹¹ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R¹¹ is —CF₃. In some embodiments, R¹¹ is —CHF₂.

In some embodiments,

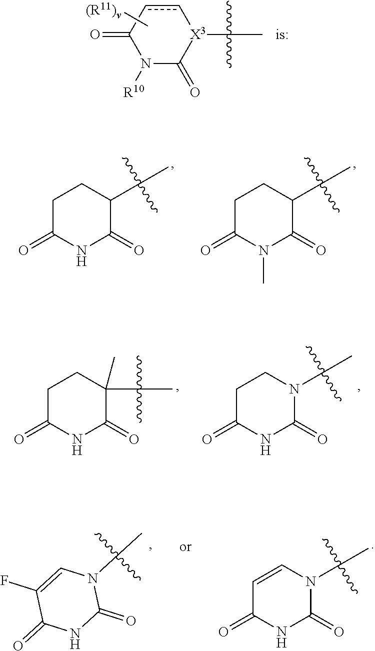

In some embodiments, the compound of Formula (I) is a compound of Formula (II):

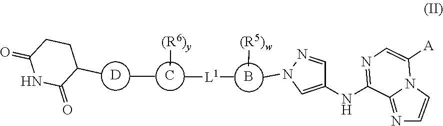

wherein A, R⁵, R⁶, w, y, L¹, Ring B, Ring C, and Ring D are as described for Formula (I).

In some embodiments, the compound of Formula (I) is a compound of Formula (IIIa), (IIIb), or (IIIc):

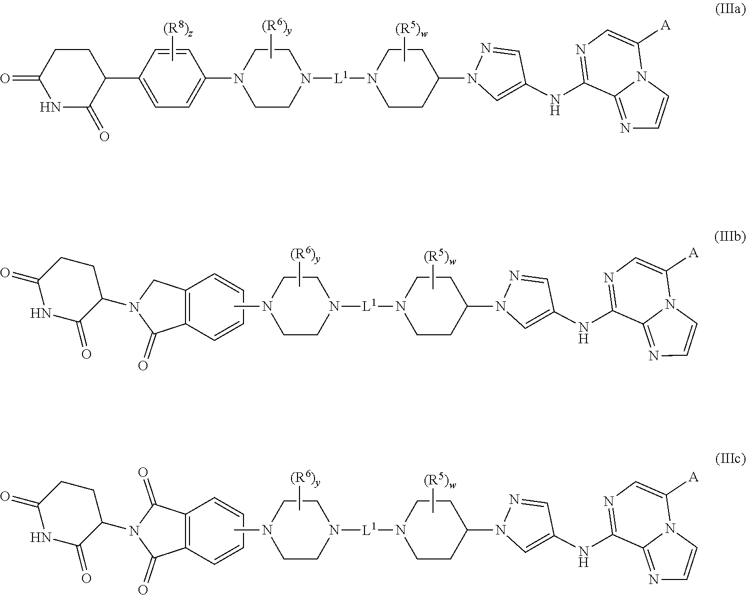

wherein A, R⁵, R⁶, R⁸, w, y, z, L are as described for Formula (I).

In some embodiments, the compound of Formula (I) is a compound of Formula (IVa) or (IVb):

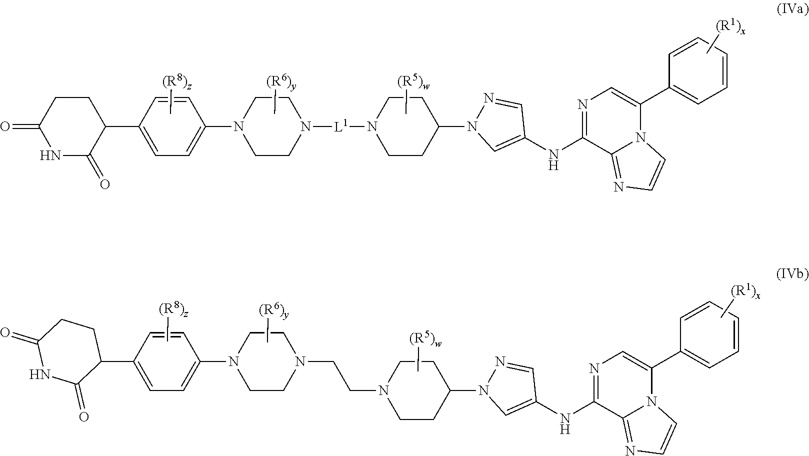

wherein R¹, R⁵, R⁶, R⁸, x, w, y, z, and L¹ are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to A of Formula (I) may be combined with every description, variation, embodiment, or aspect of R¹, R^{a}, R^{b}, R^{c}, R², R³, R⁴, R⁵, R⁶, R^{7a}, R^{7b}, R⁸, R⁹, R¹⁰, R¹¹, X¹, X², X³, L¹, Ring B, Ring C, Ring D, n, v, w, x, y, and z, the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (Ia), (II), (IIIa), (IIIb), (IIIc), (IVa), and (IVb), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | |
|---|---|
| No. | Structure |
| 1 | 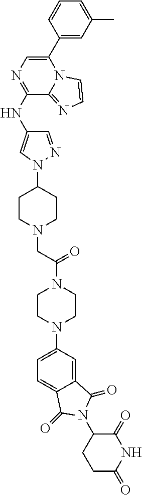 [SMILES: Cc1cccc(-c2cnc(Nc3cnn(C4CCN(CC(=O)N5CCN(c6ccc7c(c6)C(=O)N(C6CCC(=O)NC6=O)C7=O)CC5)CC4)c3)c3nccn23)c1] |
| 2 | 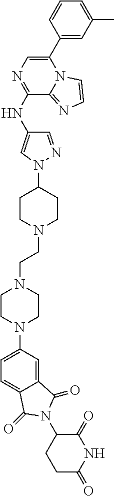 [SMILES: Cc1cccc(-c2cnc(Nc3cnn(C4CCN(CCN5CCN(c6ccc7c(c6)C(=O)N(C6CCC(=O)NC6=O)C7=O)CC5)CC4)c3)c3nccn23)c1] |
| 3 | 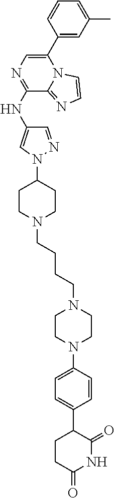 [SMILES: Cc1cccc(-c2cnc(Nc3cnn(C4CCN(CCCCN5CCN(c6ccc(C7CCC(=O)NC7=O)cc6)CC5)CC4)c3)c3nccn23)c1] |
| 4 | 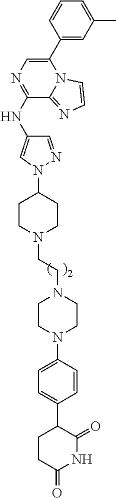 |
| 5 | 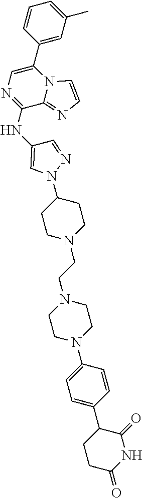 [SMILES: Cc1cccc(-c2cnc(Nc3cnn(C4CCN(CCN5CCN(c6ccc(C7CCC(=O)NC7=O)cc6)CC5)CC4)c3)c3nccn23)c1] |
| 6 | 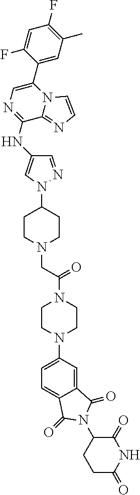 [SMILES: Cc1cc(-c2cnc(Nc3cnn(C4CCN(CC(=O)N5CCN(c6ccc7c(c6)C(=O)N(C6CCC(=O)NC6=O)C7=O)CC5)CC4)c3)c3nccn23)c(F)cc1F] |
| 7 | 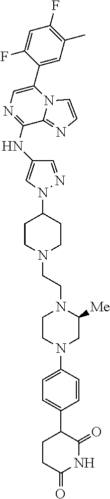 [SMILES: Cc1cc(-c2cnc(Nc3cnn(C4CCN(CCN5CCN(c6ccc(C7CCC(=O)NC7=O)cc6)C[C@@H]5C)CC4)c3)c3nccn23)c(F)cc1F] |
| 8 | 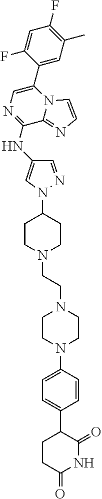 [SMILES: Cc1cc(-c2cnc(Nc3cnn(C4CCN(CCN5CCN(c6ccc(C7CCC(=O)NC7=O)cc6)CC5)CC4)c3)c3nccn23)c(F)cc1F] |
| 9 | 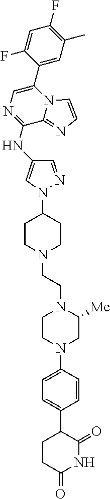 [SMILES: Cc1cc(-c2cnc(Nc3cnn(C4CCN(CCN5CCN(c6ccc(C7CCC(=O)NC7=O)cc6)C[C@H]5C)CC4)c3)c3nccn23)c(F)cc1F] |
| 10 | 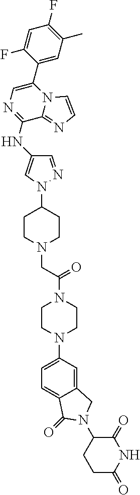 [SMILES: Cc1cc(-c2cnc(Nc3cnn(C4CCN(CC(=O)N5CCN(c6ccc7c(c6)CN(C6CCC(=O)NC6=O)C7=O)CC5)CC4)c3)c3nccn23)c(F)cc1F] |
| 11 | 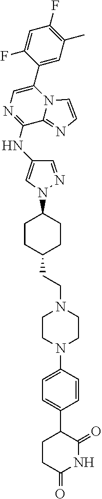 [SMILES: Cc1cc(-c2cnc(Nc3cnn([C@H]4CC[C@H](CCN5CCN(c6ccc(C7CCC(=O)NC7=O)cc6)CC5)CC4)c3)c3nccn23)c(F)cc1F] |
| 12 | 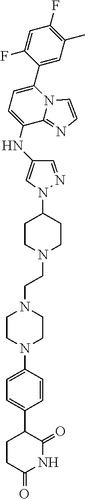 [SMILES: Cc1cc(-c2ccc(Nc3cnn(C4CCN(CCN5CCN(c6ccc(C7CCC(=O)NC7=O)cc6)CC5)CC4)c3)c3nccn23)c(F)cc1F] |
| 13 | 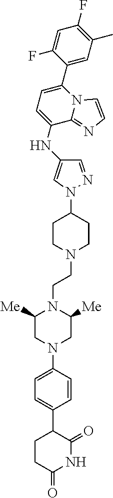 [SMILES: Cc1cc(-c2ccc(Nc3cnn(C4CCN(CCN5[C@H](C)CN(c6ccc(C7CCC(=O)NC7=O)cc6)C[C@@H]5C)CC4)c3)c3nccn23)c(F)cc1F] |
| 14 | 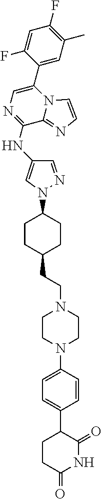 [SMILES: Cc1cc(-c2cnc(Nc3cnn([C@H]4CC[C@@H](CCN5CCN(c6ccc(C7CCC(=O)NC7=O)cc6)CC5)CC4)c3)c3nccn23)c(F)cc1F] |
| 15 | 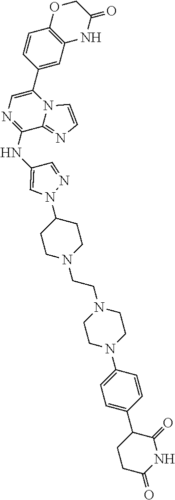 [SMILES: O=C1CCC(c2ccc(N3CCN(CCN4CCC(n5cc(Nc6ncc(-c7ccc8c(c7)NC(=O)CO8)n7ccnc67)cn5)CC4)CC3)cc2)C(=O)N1] |
| 16 | 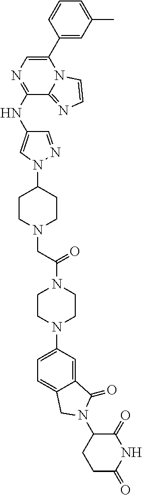 [SMILES: Cc1cccc(-c2cnc(Nc3cnn(C4CCN(CC(=O)N5CCN(c6ccc7c(c6)C(=O)N(C6CCC(=O)NC6=O)C7)CC5)CC4)c3)c3nccn23)c1] |
| 17 | 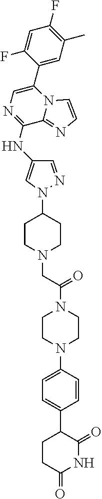 [SMILES: Cc1cc(-c2cnc(Nc3cnn(C4CCN(CC(=O)N5CCN(c6ccc(C7CCC(=O)NC7=O)cc6)CC5)CC4)c3)c3nccn23)c(F)cc1F] |
| 18 | 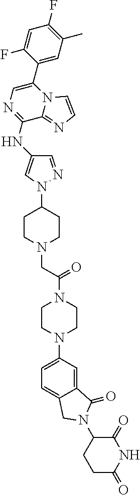 [SMILES: Cc1cc(-c2cnc(Nc3cnn(C4CCN(CC(=O)N5CCN(c6ccc7c(c6)C(=O)N(C6CCC(=O)NC6=O)C7)CC5)CC4)c3)c3nccn23)c(F)cc1F] |
| 19 | 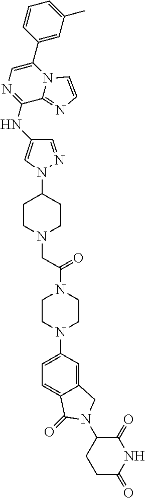 [SMILES: Cc1cccc(-c2cnc(Nc3cnn(C4CCN(CC(=O)N5CCN(c6ccc7c(c6)CN(C6CCC(=O)NC6=O)C7=O)CC5)CC4)c3)c3nccn23)c1] |
| 20 | 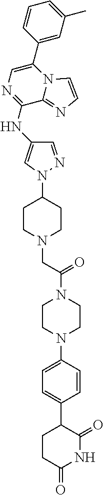 [SMILES: Cc1cccc(-c2cnc(Nc3cnn(C4CCN(CC(=O)N5CCN(c6ccc(C7CCC(=O)NC7=O)cc6)CC5)CC4)c3)c3nccn23)c1] |
| 21 | 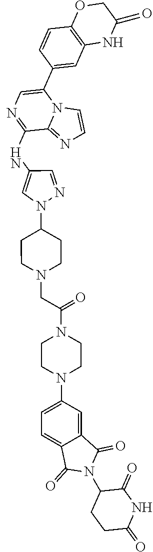 [SMILES: O=C1CCC(N2C(=O)c3ccc(N4CCN(C(=O)CN5CCC(n6cc(Nc7ncc(-c8ccc9c(c8)NC(=O)CO9)n8ccnc78)cn6)CC5)CC4)cc3C2=O)C(=O)N1] |
| 22 | 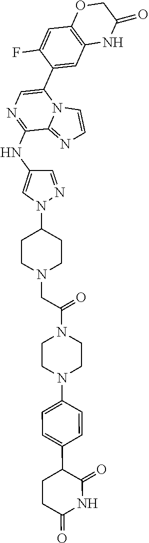 [SMILES: O=C1CCC(c2ccc(N3CCN(C(=O)CN4CCC(n5cc(Nc6ncc(-c7cc8c(cc7F)OCC(=O)N8)n7ccnc67)cn5)CC4)CC3)cc2)C(=O)N1] |
| 23 | 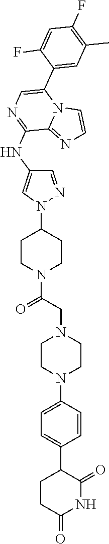 [SMILES: Cc1cc(-c2cnc(Nc3cnn(C4CCN(C(=O)CN5CCN(c6ccc(C7CCC(=O)NC7=O)cc6)CC5)CC4)c3)c3nccn23)c(F)cc1F] |
| 24 | 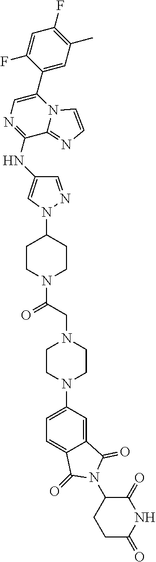 [SMILES: Cc1cc(-c2cnc(Nc3cnn(C4CCN(C(=O)CN5CCN(c6ccc7c(c6)C(=O)N(C6CCC(=O)NC6=O)C7=O)CC5)CC4)c3)c3nccn23)c(F)cc1F] |
| 25 | 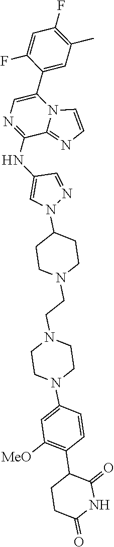 [SMILES: COc1cc(N2CCN(CCN3CCC(n4cc(Nc5ncc(-c6cc(C)c(F)cc6F)n6ccnc56)cn4)CC3)CC2)ccc1C1CCC(=O)NC1=O] |
| 26 | 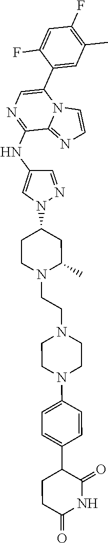 [SMILES: Cc1cc(-c2cnc(Nc3cnn([C@H]4CCN(CCN5CCN(c6ccc(C7CCC(=O)NC7=O)cc6)CC5)[C@@H](C)C4)c3)c3nccn23)c(F)cc1F] |
| 27 | 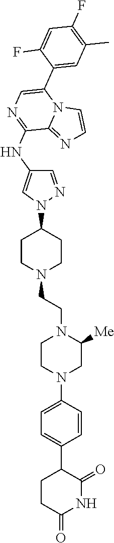 |
| 28 | 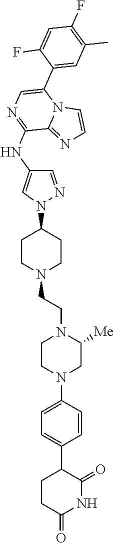 |
| 29 | 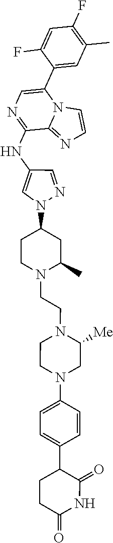 [SMILES: Cc1cc(-c2cnc(Nc3cnn([C@@H]4CCN(CCN5CCN(c6ccc(C7CCC(=O)NC7=O)cc6)C[C@H]5C)[C@H](C)C4)c3)c3nccn23)c(F)cc1F] |
| 30 | 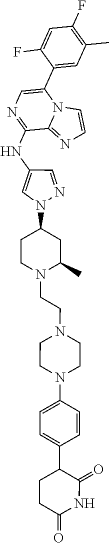 [SMILES: Cc1cc(-c2cnc(Nc3cnn([C@@H]4CCN(CCN5CCN(c6ccc(C7CCC(=O)NC7=O)cc6)CC5)[C@H](C)C4)c3)c3nccn23)c(F)cc1F] |
| 31 | 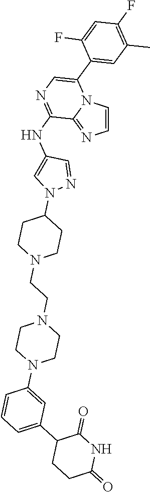 [SMILES: Cc1cc(-c2cnc(Nc3cnn(C4CCN(CCN5CCN(c6cccc(C7CCC(=O)NC7=O)c6)CC5)CC4)c3)c3nccn23)c(F)cc1F] |
| 32 | 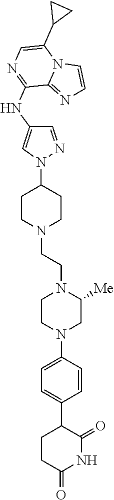 [SMILES: C[C@@H]1CN(c2ccc(C3CCC(=O)NC3=O)cc2)CCN1CCN1CCC(n2cc(Nc3ncc(C4CC4)n4ccnc34)cn2)CC1] |
| 33 | 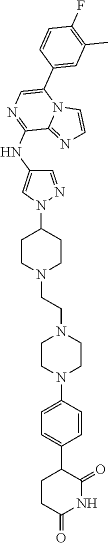 [SMILES: Cc1cc(-c2cnc(Nc3cnn(C4CCN(CCN5CCN(c6ccc(C7CCC(=O)NC7=O)cc6)CC5)CC4)c3)c3nccn23)ccc1F] |
| 34 | 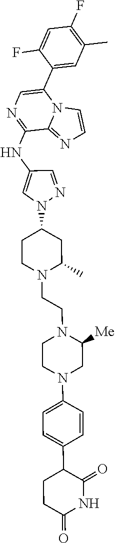 [SMILES: Cc1cc(-c2cnc(Nc3cnn([C@H]4CCN(CCN5CCN(c6ccc(C7CCC(=O)NC7=O)cc6)C[C@@H]5C)[C@@H](C)C4)c3)c3nccn23)c(F)cc1F] |
| 35 | 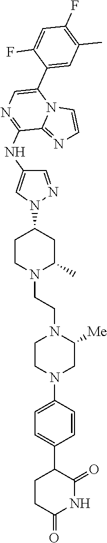 [SMILES: Cc1cc(-c2cnc(Nc3cnn([C@H]4CCN(CCN5CCN(c6ccc(C7CCC(=O)NC7=O)cc6)C[C@H]5C)[C@@H](C)C4)c3)c3nccn23)c(F)cc1F] |
| 36 | 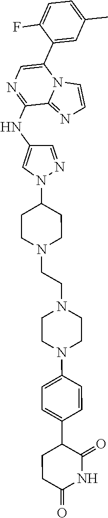 [SMILES: Cc1ccc(F)c(-c2cnc(Nc3cnn(C4CCN(CCN5CCN(c6ccc(C7CCC(=O)NC7=O)cc6)CC5)CC4)c3)c3nccn23)c1] |
| 37 | 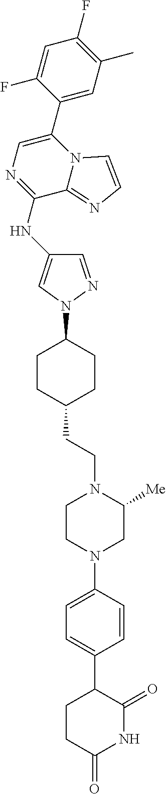 [SMILES: Cc1cc(-c2cnc(Nc3cnn([C@H]4CC[C@H](CCN5CCN(c6ccc(C7CCC(=O)NC7=O)cc6)C[C@H]5C)CC4)c3)c3nccn23)c(F)cc1F] |
| 38 | 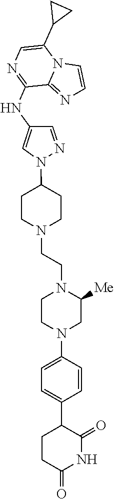 [SMILES: C[C@H]1CN(c2ccc(C3CCC(=O)NC3=O)cc2)CCN1CCN1CCC(n2cc(Nc3ncc(C4CC4)n4ccnc34)cn2)CC1] |
| 39 | 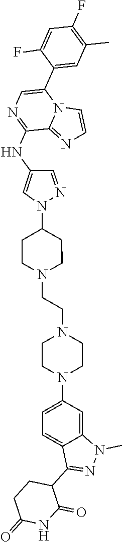 [SMILES: Cc1cc(-c2cnc(Nc3cnn(C4CCN(CCN5CCN(c6ccc7c(C8CCC(=O)NC8=O)nn(C)c7c6)CC5)CC4)c3)c3nccn23)c(F)cc1F] |
| 40 | 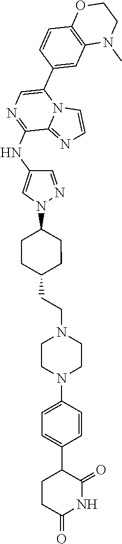 [SMILES: CN1CCOc2ccc(-c3cnc(Nc4cnn([C@H]5CC[C@H](CCN6CCN(c7ccc(C8CCC(=O)NC8=O)cc7)CC6)CC5)c4)c4nccn34)cc21] |
| 41 | 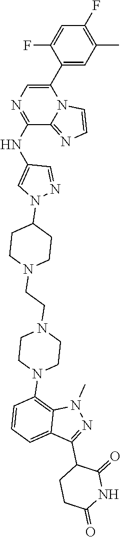 [SMILES: Cc1cc(-c2cnc(Nc3cnn(C4CCN(CCN5CCN(c6cccc7c(C8CCC(=O)NC8=O)nn(C)c67)CC5)CC4)c3)c3nccn23)c(F)cc1F] |
| 42 | 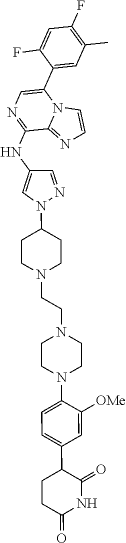 [SMILES: COc1cc(C2CCC(=O)NC2=O)ccc1N1CCN(CCN2CCC(n3cc(Nc4ncc(-c5cc(C)c(F)cc5F)n5ccnc45)cn3)CC2)CC1] |
| 43 | 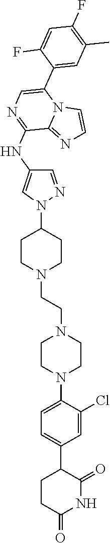 [SMILES: Cc1cc(-c2cnc(Nc3cnn(C4CCN(CCN5CCN(c6ccc(C7CCC(=O)NC7=O)cc6Cl)CC5)CC4)c3)c3nccn23)c(F)cc1F] |
| 44 | 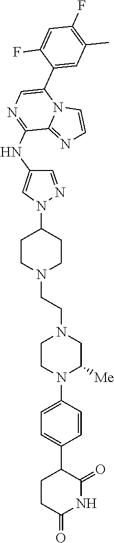 [SMILES: Cc1cc(-c2cnc(Nc3cnn(C4CCN(CCN5CCN(c6ccc(C7CCC(=O)NC7=O)cc6)[C@@H](C)C5)CC4)c3)c3nccn23)c(F)cc1F] |
| 45 | 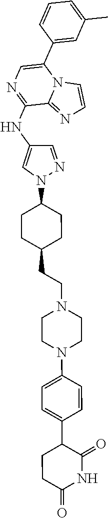 [SMILES: Cc1cccc(-c2cnc(Nc3cnn([C@H]4CC[C@@H](CCN5CCN(c6ccc(C7CCC(=O)NC7=O)cc6)CC5)CC4)c3)c3nccn23)c1] |
| 46 | 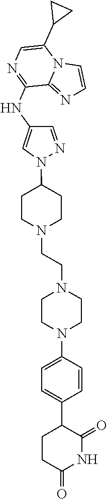 [SMILES: O=C1CCC(c2ccc(N3CCN(CCN4CCC(n5cc(Nc6ncc(C7CC7)n7ccnc67)cn5)CC4)CC3)cc2)C(=O)N1] |
| 47 | 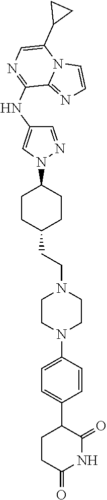 [SMILES: O=C1CCC(c2ccc(N3CCN(CC[C@H]4CC[C@H](n5cc(Nc6ncc(C7CC7)n7ccnc67)cn5)CC4)CC3)cc2)C(=O)N1] |
| 48 | 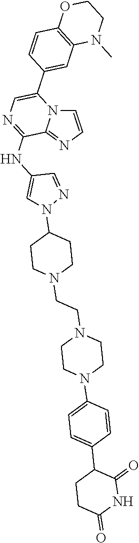 [SMILES: CN1CCOc2ccc(-c3cnc(Nc4cnn(C5CCN(CCN6CCN(c7ccc(C8CCC(=O)NC8=O)cc7)CC6)CC5)c4)c4nccn34)cc21] |
| 49 | 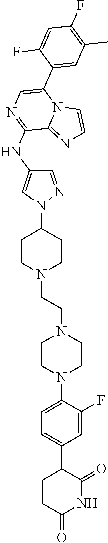 [SMILES: Cc1cc(-c2cnc(Nc3cnn(C4CCN(CCN5CCN(c6ccc(C7CCC(=O)NC7=O)cc6F)CC5)CC4)c3)c3nccn23)c(F)cc1F] |
| 50 | 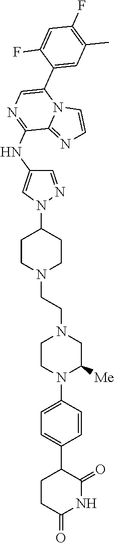 [SMILES: Cc1cc(-c2cnc(Nc3cnn(C4CCN(CCN5CCN(c6ccc(C7CCC(=O)NC7=O)cc6)[C@H](C)C5)CC4)c3)c3nccn23)c(F)cc1F] |
| 51 | 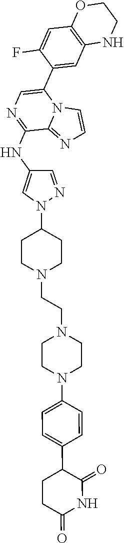 [SMILES: O=C1CCC(c2ccc(N3CCN(CCN4CCC(n5cc(Nc6ncc(-c7cc8c(cc7F)OCCN8)n7ccnc67)cn5)CC4)CC3)cc2)C(=O)N1] |
| 52 | 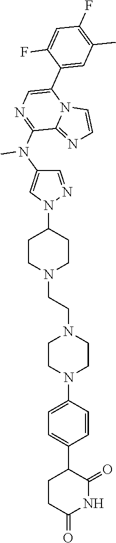 |
| 53 | 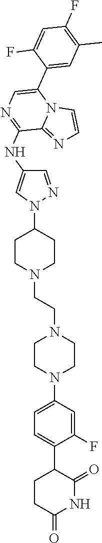 [SMILES: Cc1cc(-c2cnc(Nc3cnn(C4CCN(CCN5CCN(c6ccc(C7CCC(=O)NC7=O)c(F)c6)CC5)CC4)c3)c3nccn23)c(F)cc1F] |
| 54 | 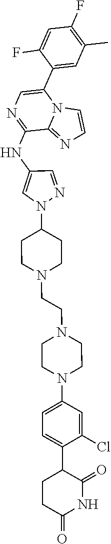 [SMILES: Cc1cc(-c2cnc(Nc3cnn(C4CCN(CCN5CCN(c6ccc(C7CCC(=O)NC7=O)c(Cl)c6)CC5)CC4)c3)c3nccn23)c(F)cc1F] |
| 55 | 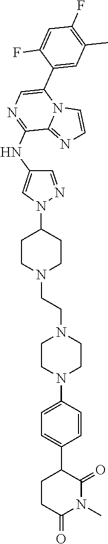 [SMILES: Cc1cc(-c2cnc(Nc3cnn(C4CCN(CCN5CCN(c6ccc(C7CCC(=O)N(C)C7=O)cc6)CC5)CC4)c3)c3nccn23)c(F)cc1F] |
| 56 | 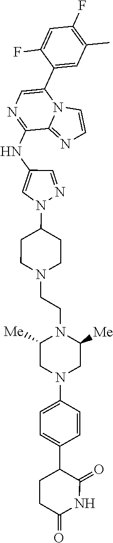 [SMILES: Cc1cc(-c2cnc(Nc3cnn(C4CCN(CCN5[C@@H](C)CN(c6ccc(C7CCC(=O)NC7=O)cc6)C[C@@H]5C)CC4)c3)c3nccn23)c(F)cc1F] |
| 57 | 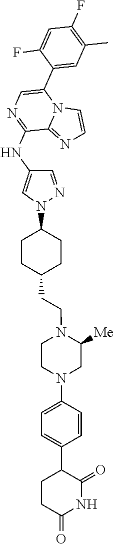 [SMILES: Cc1cc(-c2cnc(Nc3cnn([C@H]4CC[C@H](CCN5CCN(c6ccc(C7CCC(=O)NC7=O)cc6)C[C@@H]5C)CC4)c3)c3nccn23)c(F)cc1F] |
| 58 | 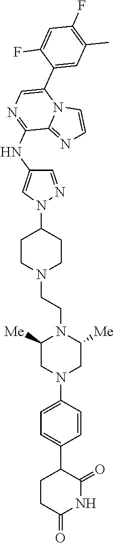 [SMILES: Cc1cc(-c2cnc(Nc3cnn(C4CCN(CCN5[C@H](C)CN(c6ccc(C7CCC(=O)NC7=O)cc6)C[C@H]5C)CC4)c3)c3nccn23)c(F)cc1F] |
| 59 | 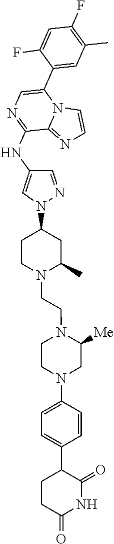 [SMILES: Cc1cc(-c2cnc(Nc3cnn([C@@H]4CCN(CCN5CCN(c6ccc(C7CCC(=O)NC7=O)cc6)C[C@@H]5C)[C@H](C)C4)c3)c3nccn23)c(F)cc1F] |
| 60 | 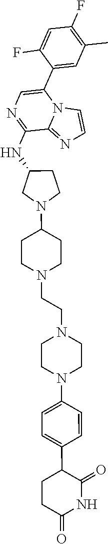 [SMILES: Cc1cc(-c2cnc(N[C@@H]3CCN(C4CCN(CCN5CCN(c6ccc(C7CCC(=O)NC7=O)cc6)CC5)CC4)C3)c3nccn23)c(F)cc1F] |
| 61 | 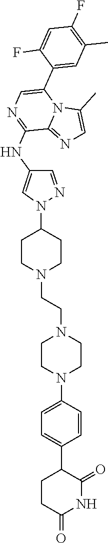 [SMILES: Cc1cc(-c2cnc(Nc3cnn(C4CCN(CCN5CCN(c6ccc(C7CCC(=O)NC7=O)cc6)CC5)CC4)c3)c3ncc(C)n23)c(F)cc1F] |
| 62 | 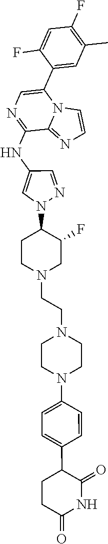 [SMILES: Cc1cc(-c2cnc(Nc3cnn([C@@H]4CCN(CCN5CCN(c6ccc(C7CCC(=O)NC7=O)cc6)CC5)C[C@H]4F)c3)c3nccn23)c(F)cc1F] |
| 63 | 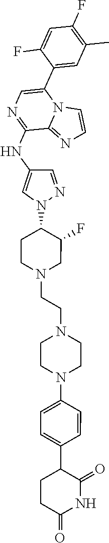 [SMILES: Cc1cc(-c2cnc(Nc3cnn([C@H]4CCN(CCN5CCN(c6ccc(C7CCC(=O)NC7=O)cc6)CC5)C[C@H]4F)c3)c3nccn23)c(F)cc1F] |
| 64 | 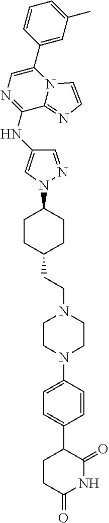 [SMILES: Cc1cccc(-c2cnc(Nc3cnn([C@H]4CC[C@H](CCN5CCN(c6ccc(C7CCC(=O)NC7=O)cc6)CC5)CC4)c3)c3nccn23)c1] |
| 65 | 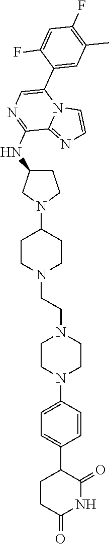 [SMILES: Cc1cc(-c2cnc(N[C@H]3CCN(C4CCN(CCN5CCN(c6ccc(C7CCC(=O)NC7=O)cc6)CC5)CC4)C3)c3nccn23)c(F)cc1F] |
| 66 | 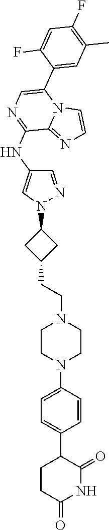 [SMILES: Cc1cc(-c2cnc(Nc3cnn([C@H]4C[C@H](CCN5CCN(c6ccc(C7CCC(=O)NC7=O)cc6)CC5)C4)c3)c3nccn23)c(F)cc1F] |
| 67 | 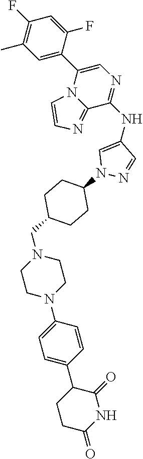 [SMILES: Cc1cc(-c2cnc(Nc3cnn([C@H]4CC[C@H](CN5CCN(c6ccc(C7CCC(=O)NC7=O)cc6)CC5)CC4)c3)c3nccn23)c(F)cc1F] |
| 68 | 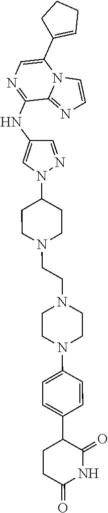 [SMILES: O=C1CCC(c2ccc(N3CCN(CCN4CCC(n5cc(Nc6ncc(C7=CCCC7)n7ccnc67)cn5)CC4)CC3)cc2)C(=O)N1] |
| 69 | 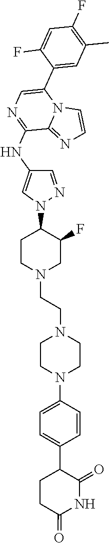 [SMILES: Cc1cc(-c2cnc(Nc3cnn([C@@H]4CCN(CCN5CCN(c6ccc(C7CCC(=O)NC7=O)cc6)CC5)C[C@@H]4F)c3)c3nccn23)c(F)cc1F] |
| 70 | 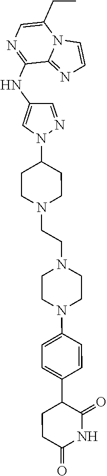 [SMILES: CCc1cnc(Nc2cnn(C3CCN(CCN4CCN(c5ccc(C6CCC(=O)NC6=O)cc5)CC4)CC3)c2)c2nccn12] |
| 71 | 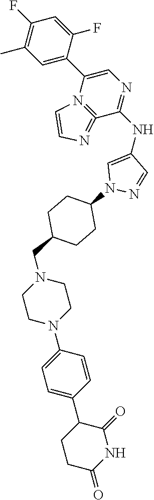 |
| 72 | 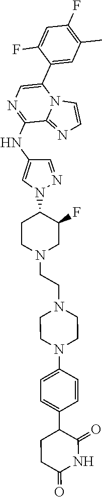 [SMILES: Cc1cc(-c2cnc(Nc3cnn([C@H]4CCN(CCN5CCN(c6ccc(C7CCC(=O)NC7=O)cc6)CC5)C[C@@H]4F)c3)c3nccn23)c(F)cc1F] |
| 73 | 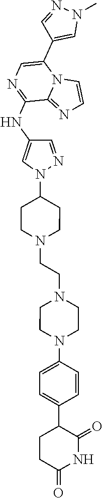 [SMILES: Cn1cc(-c2cnc(Nc3cnn(C4CCN(CCN5CCN(c6ccc(C7CCC(=O)NC7=O)cc6)CC5)CC4)c3)c3nccn23)cn1] |
| 74 | 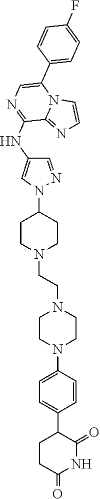 [SMILES: O=C1CCC(c2ccc(N3CCN(CCN4CCC(n5cc(Nc6ncc(-c7ccc(F)cc7)n7ccnc67)cn5)CC4)CC3)cc2)C(=O)N1] |
| 75 | 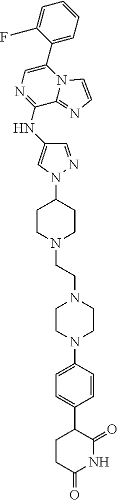 [SMILES: O=C1CCC(c2ccc(N3CCN(CCN4CCC(n5cc(Nc6ncc(-c7ccccc7F)n7ccnc67)cn5)CC4)CC3)cc2)C(=O)N1] |
| 76 | 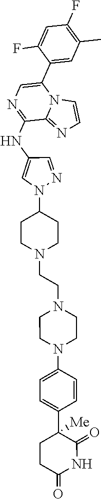 [SMILES: Cc1cc(-c2cnc(Nc3cnn(C4CCN(CCN5CCN(c6ccc([C@]7(C)CCC(=O)NC7=O)cc6)CC5)CC4)c3)c3nccn23)c(F)cc1F] |
| 77 | 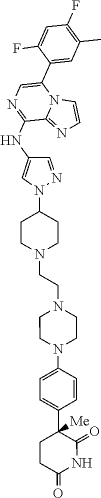 [SMILES: Cc1cc(-c2cnc(Nc3cnn(C4CCN(CCN5CCN(c6ccc([C@@]7(C)CCC(=O)NC7=O)cc6)CC5)CC4)c3)c3nccn23)c(F)cc1F] |
| 78 | 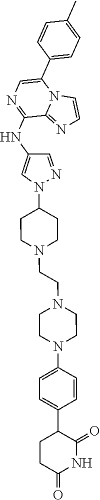 [SMILES: Cc1ccc(-c2cnc(Nc3cnn(C4CCN(CCN5CCN(c6ccc(C7CCC(=O)NC7=O)cc6)CC5)CC4)c3)c3nccn23)cc1] |
| 79 | 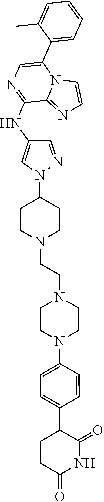 [SMILES: Cc1ccccc1-c1cnc(Nc2cnn(C3CCN(CCN4CCN(c5ccc(C6CCC(=O)NC6=O)cc5)CC4)CC3)c2)c2nccn12] |
| 80 | 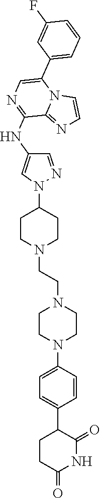 [SMILES: O=C1CCC(c2ccc(N3CCN(CCN4CCC(n5cc(Nc6ncc(-c7cccc(F)c7)n7ccnc67)cn5)CC4)CC3)cc2)C(=O)N1] |
| 81 | 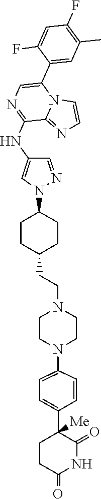 [SMILES: Cc1cc(-c2cnc(Nc3cnn([C@H]4CC[C@H](CCN5CCN(c6ccc([C@@]7(C)CCC(=O)NC7=O)cc6)CC5)CC4)c3)c3nccn23)c(F)cc1F] |
| 82 | 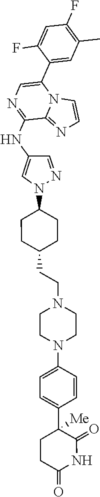 [SMILES: Cc1cc(-c2cnc(Nc3cnn([C@H]4CC[C@H](CCN5CCN(c6ccc([C@]7(C)CCC(=O)NC7=O)cc6)CC5)CC4)c3)c3nccn23)c(F)cc1F] |
| 83 | 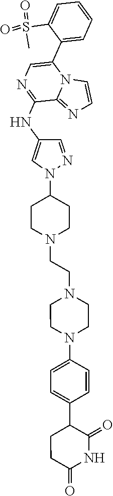 [SMILES: CS(=O)(=O)c1ccccc1-c1cnc(Nc2cnn(C3CCN(CCN4CCN(c5ccc(C6CCC(=O)NC6=O)cc5)CC4)CC3)c2)c2nccn12] |
| 84 | 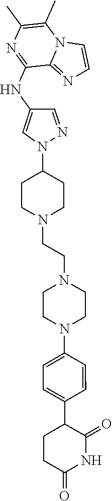 [SMILES: Cc1nc(Nc2cnn(C3CCN(CCN4CCN(c5ccc(C6CCC(=O)NC6=O)cc5)CC4)CC3)c2)c2nccn2c1C] |
| 85 | 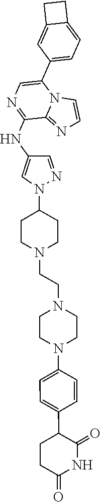 [SMILES: O=C1CCC(c2ccc(N3CCN(CCN4CCC(n5cc(Nc6ncc(-c7ccc8c(c7)CC8)n7ccnc67)cn5)CC4)CC3)cc2)C(=O)N1] |
| 86 | 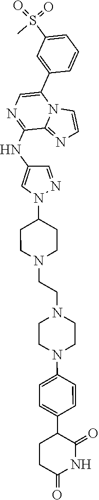 [SMILES: CS(=O)(=O)c1cccc(-c2cnc(Nc3cnn(C4CCN(CCN5CCN(c6ccc(C7CCC(=O)NC7=O)cc6)CC5)CC4)c3)c3nccn23)c1] |
| 87 | 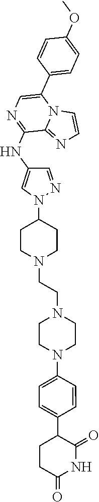 [SMILES: COc1ccc(-c2cnc(Nc3cnn(C4CCN(CCN5CCN(c6ccc(C7CCC(=O)NC7=O)cc6)CC5)CC4)c3)c3nccn23)cc1] |
| 88 | 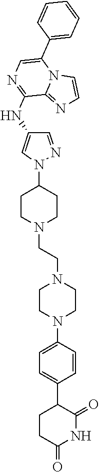 |
| 89 | 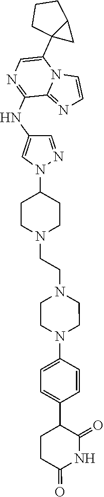 [SMILES: O=C1CCC(c2ccc(N3CCN(CCN4CCC(n5cc(Nc6ncc(C78CCCC7C8)n7ccnc67)cn5)CC4)CC3)cc2)C(=O)N1] |
| 90 | 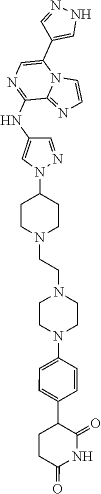 [SMILES: O=C1CCC(c2ccc(N3CCN(CCN4CCC(n5cc(Nc6ncc(-c7cn[nH]c7)n7ccnc67)cn5)CC4)CC3)cc2)C(=O)N1] |
| 91 | 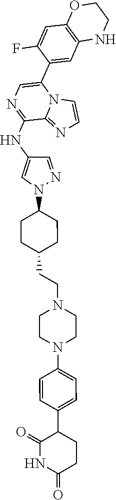 [SMILES: O=C1CCC(c2ccc(N3CCN(CC[C@H]4CC[C@H](n5cc(Nc6ncc(-c7cc8c(cc7F)OCCN8)n7ccnc67)cn5)CC4)CC3)cc2)C(=O)N1] |
| 92 | 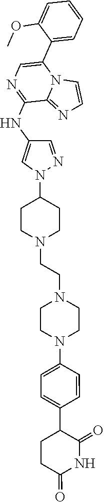 [SMILES: COc1ccccc1-c1cnc(Nc2cnn(C3CCN(CCN4CCN(c5ccc(C6CCC(=O)NC6=O)cc5)CC4)CC3)c2)c2nccn12] |
| 93 | 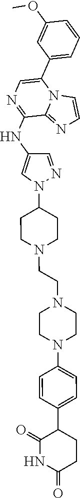 [SMILES: COc1cccc(-c2cnc(Nc3cnn(C4CCN(CCN5CCN(c6ccc(C7CCC(=O)NC7=O)cc6)CC5)CC4)c3)c3nccn23)c1] |
| 94 | 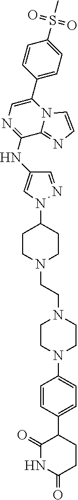 [SMILES: CS(=O)(=O)c1ccc(-c2cnc(Nc3cnn(C4CCN(CCN5CCN(c6ccc(C7CCC(=O)NC7=O)cc6)CC5)CC4)c3)c3nccn23)cc1] |
| 95 | 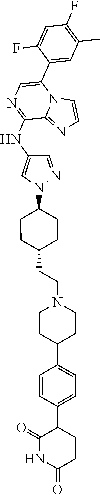 [SMILES: Cc1cc(-c2cnc(Nc3cnn([C@H]4CC[C@H](CCN5CCC(c6ccc(C7CCC(=O)NC7=O)cc6)CC5)CC4)c3)c3nccn23)c(F)cc1F] |
| 96 | 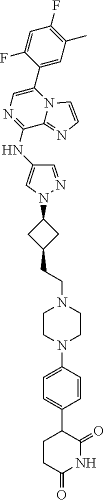 [SMILES: Cc1cc(-c2cnc(Nc3cnn([C@H]4C[C@@H](CCN5CCN(c6ccc(C7CCC(=O)NC7=O)cc6)CC5)C4)c3)c3nccn23)c(F)cc1F] |
| 97 | 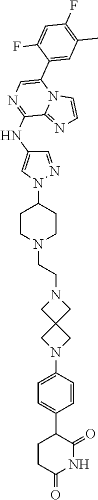 [SMILES: Cc1cc(-c2cnc(Nc3cnn(C4CCN(CCN5CC6(C5)CN(c5ccc(C7CCC(=O)NC7=O)cc5)C6)CC4)c3)c3nccn23)c(F)cc1F] |
| 98 | 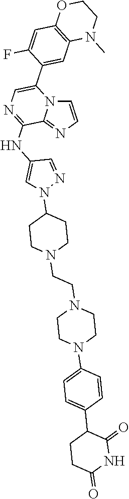 [SMILES: CN1CCOc2cc(F)c(-c3cnc(Nc4cnn(C5CCN(CCN6CCN(c7ccc(C8CCC(=O)NC8=O)cc7)CC6)CC5)c4)c4nccn34)cc21] |
| 99 | 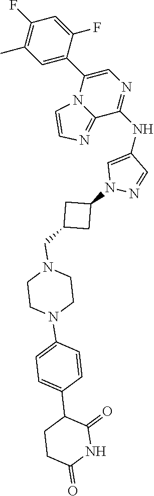 [SMILES: Cc1cc(-c2cnc(Nc3cnn([C@H]4C[C@H](CN5CCN(c6ccc(C7CCC(=O)NC7=O)cc6)CC5)C4)c3)c3nccn23)c(F)cc1F] |
| 100 | 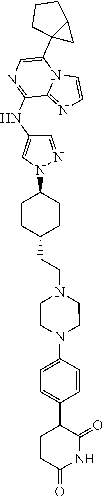 [SMILES: O=C1CCC(c2ccc(N3CCN(CC[C@H]4CC[C@H](n5cc(Nc6ncc(C78CCCC7C8)n7ccnc67)cn5)CC4)CC3)cc2)C(=O)N1] |
| 101 | 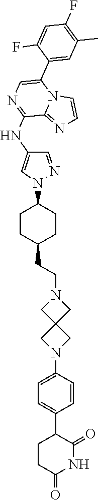 [SMILES: Cc1cc(-c2cnc(Nc3cnn([C@H]4CC[C@@H](CCN5CC6(C5)CN(c5ccc(C7CCC(=O)NC7=O)cc5)C6)CC4)c3)c3nccn23)c(F)cc1F] |
| 102 | 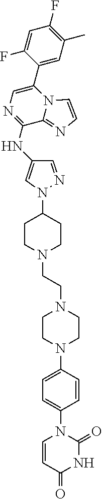 [SMILES: Cc1cc(-c2cnc(Nc3cnn(C4CCN(CCN5CCN(c6ccc(-n7ccc(=O)[nH]c7=O)cc6)CC5)CC4)c3)c3nccn23)c(F)cc1F] |
| 103 | 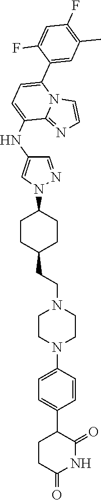 [SMILES: Cc1cc(-c2ccc(Nc3cnn([C@H]4CC[C@@H](CCN5CCN(c6ccc(C7CCC(=O)NC7=O)cc6)CC5)CC4)c3)c3nccn23)c(F)cc1F] |
| 104 | 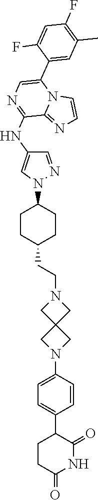 [SMILES: Cc1cc(-c2cnc(Nc3cnn([C@H]4CC[C@H](CCN5CC6(C5)CN(c5ccc(C7CCC(=O)NC7=O)cc5)C6)CC4)c3)c3nccn23)c(F)cc1F] |
| 105 | 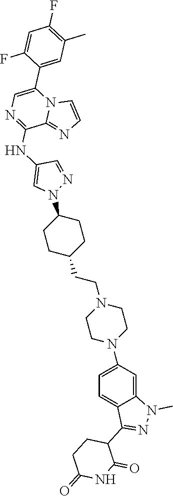 [SMILES: Cc1cc(-c2cnc(Nc3cnn([C@H]4CC[C@H](CCN5CCN(c6ccc7c(C8CCC(=O)NC8=O)nn(C)c7c6)CC5)CC4)c3)c3nccn23)c(F)cc1F] |
| 106 | 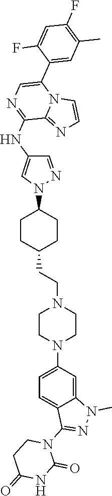 [SMILES: Cc1cc(-c2cnc(Nc3cnn([C@H]4CC[C@H](CCN5CCN(c6ccc7c(N8CCC(=O)NC8=O)nn(C)c7c6)CC5)CC4)c3)c3nccn23)c(F)cc1F] |
| 107 | 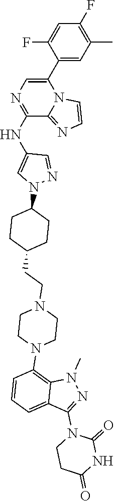 [SMILES: Cc1cc(-c2cnc(Nc3cnn([C@H]4CC[C@H](CCN5CCN(c6cccc7c(N8CCC(=O)NC8=O)nn(C)c67)CC5)CC4)c3)c3nccn23)c(F)cc1F] |
| 108 | 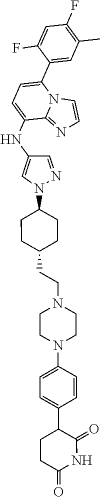 [SMILES: Cc1cc(-c2ccc(Nc3cnn([C@H]4CC[C@H](CCN5CCN(c6ccc(C7CCC(=O)NC7=O)cc6)CC5)CC4)c3)c3nccn23)c(F)cc1F] |
| 109 | 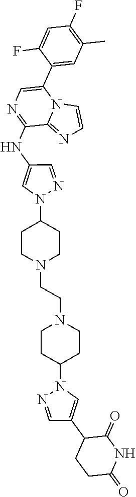 [SMILES: Cc1cc(-c2cnc(Nc3cnn(C4CCN(CCN5CCC(n6cc(C7CCC(=O)NC7=O)cn6)CC5)CC4)c3)c3nccn23)c(F)cc1F] |
| 110 | 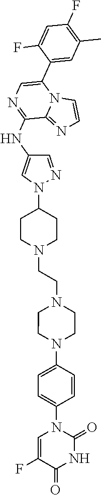 [SMILES: Cc1cc(-c2cnc(Nc3cnn(C4CCN(CCN5CCN(c6ccc(-n7cc(F)c(=O)[nH]c7=O)cc6)CC5)CC4)c3)c3nccn23)c(F)cc1F] |
| 111 | 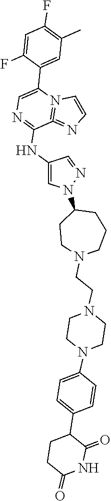 |
| 112 | 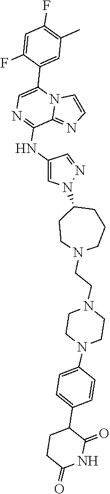 [SMILES: Cc1cc(-c2cnc(Nc3cnn([C@@H]4CCCN(CCN5CCN(c6ccc(C7CCC(=O)NC7=O)cc6)CC5)CC4)c3)c3nccn23)c(F)cc1F] |
| 113 | 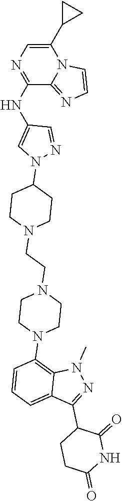 [SMILES: Cn1nc(C2CCC(=O)NC2=O)c2cccc(N3CCN(CCN4CCC(n5cc(Nc6ncc(C7CC7)n7ccnc67)cn5)CC4)CC3)c21] |
| 114 | 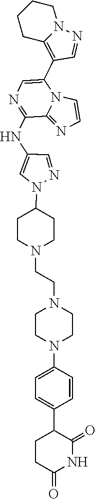 [SMILES: O=C1CCC(c2ccc(N3CCN(CCN4CCC(n5cc(Nc6ncc(-c7cnn8c7CCCC8)n7ccnc67)cn5)CC4)CC3)cc2)C(=O)N1] |
| 115 | 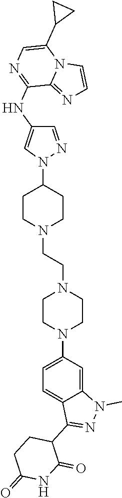 [SMILES: Cn1nc(C2CCC(=O)NC2=O)c2ccc(N3CCN(CCN4CCC(n5cc(Nc6ncc(C7CC7)n7ccnc67)cn5)CC4)CC3)cc21] |
| 116 | 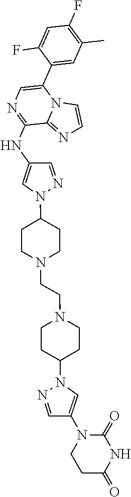 [SMILES: Cc1cc(-c2cnc(Nc3cnn(C4CCN(CCN5CCC(n6cc(N7CCC(=O)NC7=O)cn6)CC5)CC4)c3)c3nccn23)c(F)cc1F] |
| 117 | 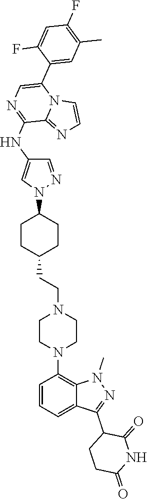 [SMILES: Cc1cc(-c2cnc(Nc3cnn([C@H]4CC[C@H](CCN5CCN(c6cccc7c(C8CCC(=O)NC8=O)nn(C)c67)CC5)CC4)c3)c3nccn23)c(F)cc1F] |
| 118 | 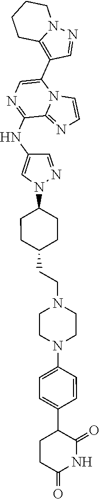 [SMILES: O=C1CCC(c2ccc(N3CCN(CC[C@H]4CC[C@H](n5cc(Nc6ncc(-c7cnn8c7CCCC8)n7ccnc67)cn5)CC4)CC3)cc2)C(=O)N1] |
| 119 | 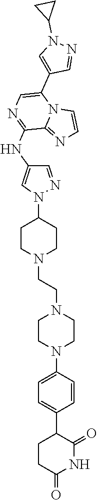 [SMILES: O=C1CCC(c2ccc(N3CCN(CCN4CCC(n5cc(Nc6ncc(-c7cnn(C8CC8)c7)n7ccnc67)cn5)CC4)CC3)cc2)C(=O)N1] |
| 120 | 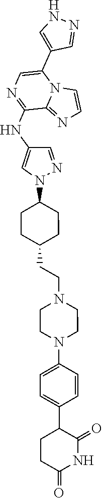 [SMILES: O=C1CCC(c2ccc(N3CCN(CC[C@H]4CC[C@H](n5cc(Nc6ncc(-c7cn[nH]c7)n7ccnc67)cn5)CC4)CC3)cc2)C(=O)N1] |

or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Schemes 1-14, as well as in the examples set forth herein. It should be noted that one skilled in the art would know how to modify the procedures set forth in the illustrative schemes and examples to arrive at the desired products.

Compounds of Formula (I) can be prepared from two main building blocks: the Target-Binding Moieties (TBMs) and the Cereblon-Binding Moieties (CBMs). General synthetic routes for preparing the TBMs and CBMs are outlined in Schemes 1-8. General synthetic routes for reacting the TBMs and CBMs to form heterobifunctional molecules (i.e., Ligand-Directed Degraders, LDDs), which are compounds of Formula (I), are provided in Schemes 9-14.

### Intermediate Compounds

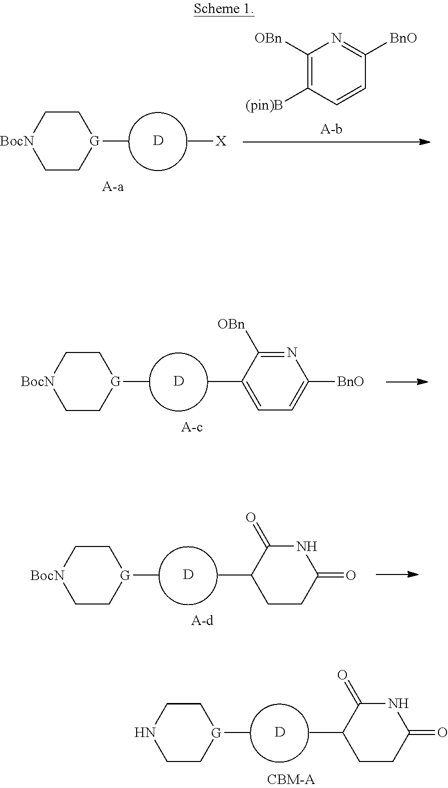

wherein Ring D is phenyl or pyrazole; G is N or CH; X is halo such as Br; and Bn is benzyl.

Scheme 1 illustrates an approach to the synthesis of intermediate compounds CBM-A. Intermediates A-a and A-b can be coupled in the presence of a palladium catalyst to yield intermediate A-c. Conversion of intermediate A-c to A-d, followed by deprotection affords intermediate compounds CBM-A.

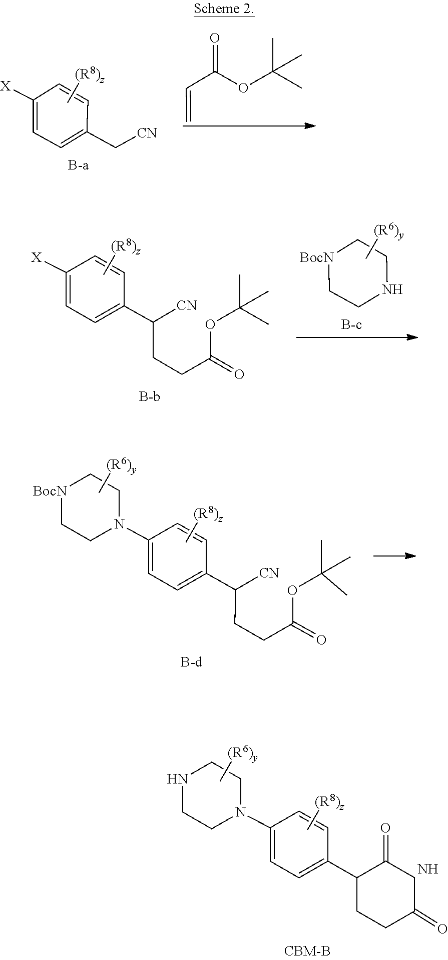

wherein X is halo such as Br; and R⁶, R⁸, y, and z are as described for Formula (I).

Scheme 2 illustrates an approach to the synthesis of intermediate compounds CBM-B. Intermediate B-a and tert-butyl acrylate can be coupled in the presence of a base such as Cs₂CO₃ or K₂CO₃ to yield intermediate B-b, which can be further coupled with intermediate B-c in the presence of a palladium catalyst to yield intermediate B-d. An internal ring formation of B-d under acidic conditions affords intermediate compounds CBM-B.

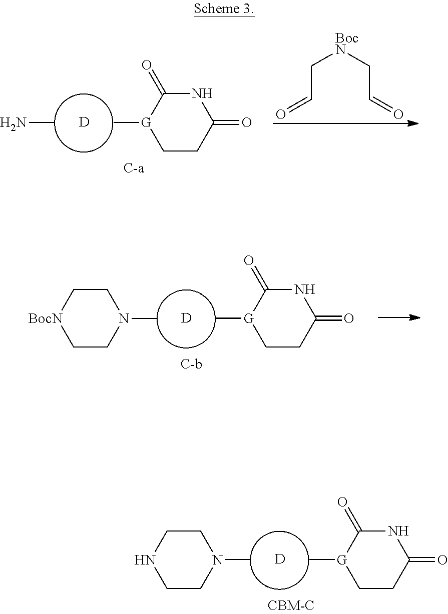

wherein Ring D is phenyl or indazole, and G is N or C(CH₃).

Scheme 3 illustrates an approach to the synthesis of intermediate compounds CBM-C. Intermediates C-a and tert-butyl bis(2-oxoethyl)carbamate can be coupled in the presence of a reducing agent to yield intermediate C-b. Deprotection of intermediate C-b under acidic conditions affords intermediate compounds CBM-C.

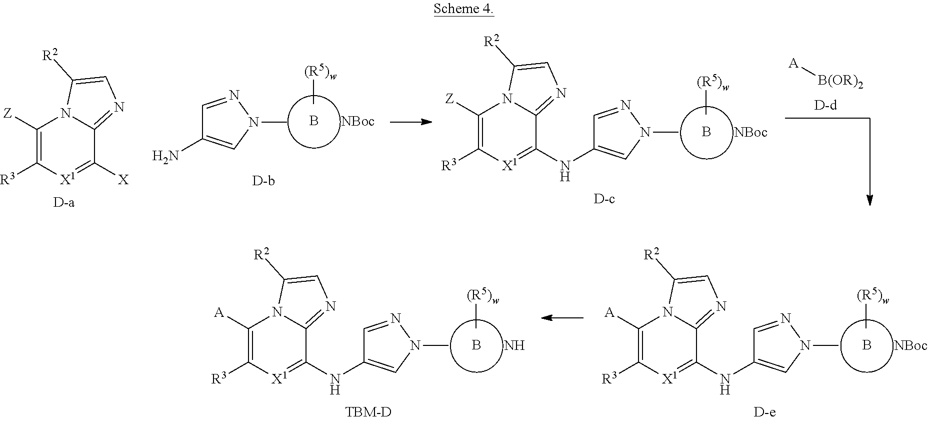

wherein Z is CH₃, Cl, or Br; X is halo such as Cl, Br, or I; R is Hand A, R², R³, R⁵, X¹, Ring B, and w are as described for Formula (I).

Scheme 4 illustrates an approach to the synthesis of intermediate compounds TBM-D. Intermediates D-a and D-b can be coupled under acidic conditions to yield intermediate D-c. Palladium catalyzed coupling of intermediates D-c and D-d then yields intermediate D-e, which is subsequently deprotected under acidic conditions to afford intermediate compounds TBM-D.

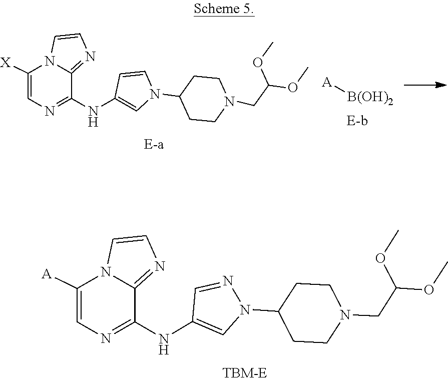

wherein X is halo such as Br; and A is as described for Formula (I).

Scheme 5 illustrates an approach to the synthesis of intermediate compounds TBM-E. Intermediates E-a and E-b can be coupled in the presence of a palladium catalyst to afford intermediate compounds TBM-E.

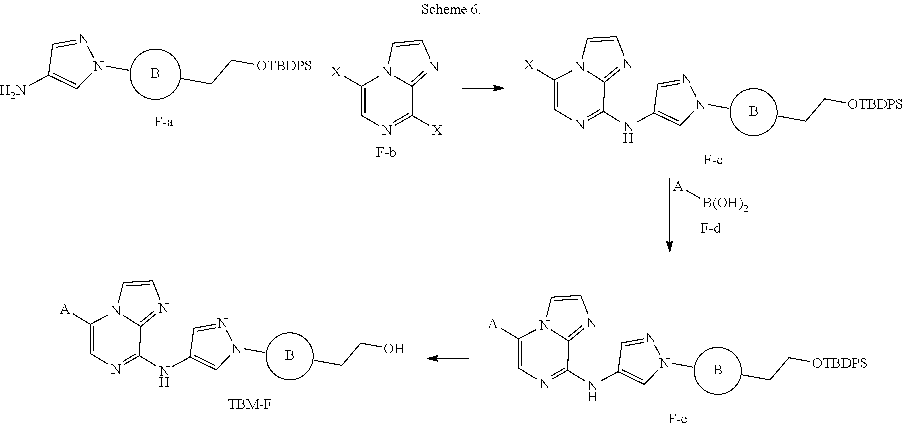

wherein each X is independently halo such as Br or Cl; and A and Ring B are as described for Formula (I).

Scheme 6 illustrates an approach to the synthesis of intermediate compounds TBM-F. Intermediates F-a and F-b can be coupled under acidic conditions to yield intermediate F-c. Palladium catalyzed coupling of intermediates F-c and F-d yields intermediate F-e, which is then deprotected under acidic conditions to afford intermediate compounds TBM-F.

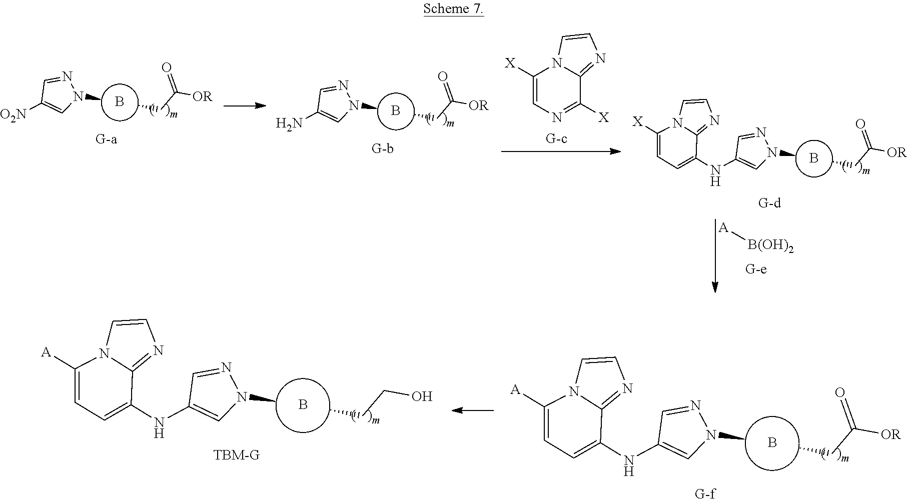

wherein each X is independently halo such as Br or Cl; R is alkyl; m is 0 or 1; Ring B is cycloalkylene; and A is as described for Formula (I).

Scheme 7 illustrates an approach to the synthesis of intermediate compounds TBM-G. Intermediate G-a can be reduced in the presence of a palladium catalyst to yield intermediate G-b. Coupling of intermediates G-b and G-c under acidic conditions yields intermediate G-d, which subsequently undergoes a palladium catalyzed coupling reaction with intermediate G-e to yield intermediate G-f. Conversion of the acid group of intermediate G-f to an alcohol group affords intermediate compounds TBM-G.

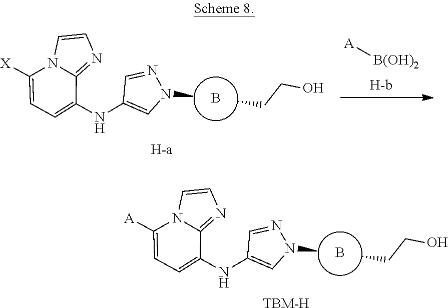

wherein X is halo such as Br; and A and Ring B are as described for Formula (I).

Scheme 8 illustrates an approach to the synthesis of intermediate compounds TBM-H. Intermediates H-a and H-b can be coupled under basic conditions with a palladium catalyst to afford intermediate compounds TBM-H.

### Final Compounds

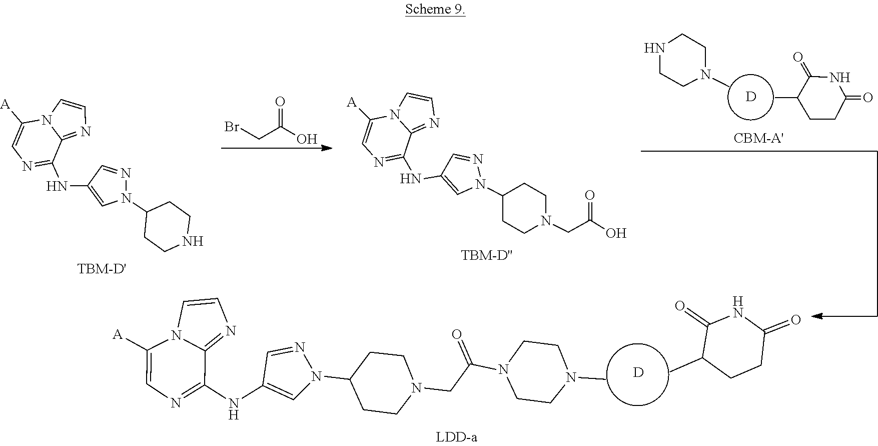

wherein A and Ring D are as described for Formula (I).

Scheme 9 illustrates an approach to the synthesis of compounds LDD-a. Coupling of TBM-D′ (a derivative of intermediate TBM-D of Scheme 4) and 2-bromoacetic acid under basic conditions can yield TBM-D″, which can be further coupled with CBM-A′ (a derivative of intermediate CBM-A of Scheme 1) under basic conditions to afford compounds LDD-a, which are compounds of Formula (I).

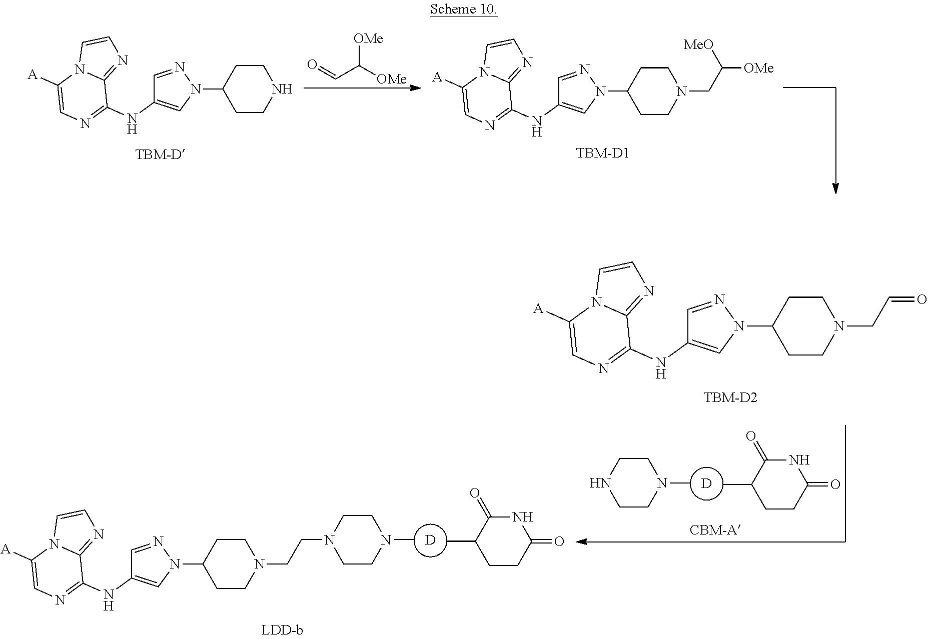

wherein A and Ring D are as described for Formula (I).

Scheme 10 illustrates an approach to the synthesis of compounds LDD-b. Coupling of TBM-D′ (a derivative of intermediate TBM-D of Scheme 4) and 2,2-dimethoxyacetaldehyde under basic conditions and with a reducing agent can yield TBM-D1. Treatment of TBM-D1 with a strong acid yields TBM-D2, which can be coupled with CBM-A′ (a derivative of intermediate CBM-A of Scheme 1) under basic conditions and with a reducing agent to afford compounds LDD-b, which are compounds of Formula (I).

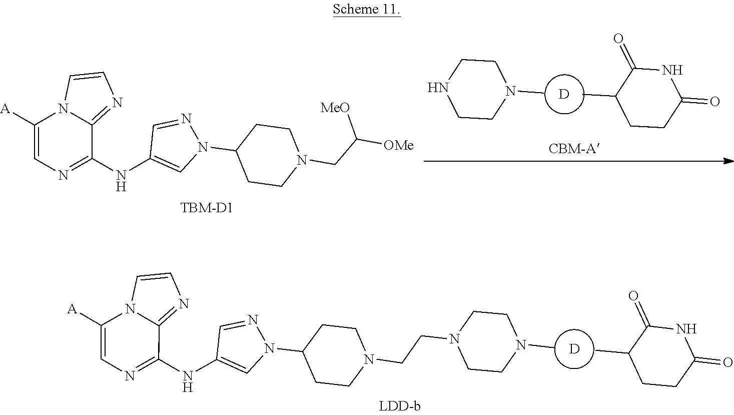

wherein A and Ring D are as described for Formula (I).

Scheme 11 illustrates an alternate synthesis of compounds LDD-b. TBM-D1 can be reacted with strong acid and subsequently coupled with CBM-A′ under basic conditions and with a reducing agent to afford compounds LDD-b, which are compounds of Formula (I).

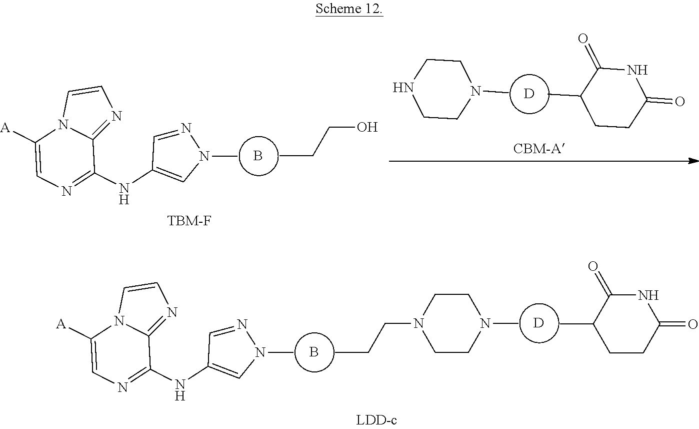

wherein A, Ring B, and Ring D are as described for Formula (I).

Scheme 12 illustrates an approach to the synthesis of compounds LDD-c. The alcohol group of TBM-F (see Scheme 6) can be converted to a leaving group and subsequently coupled with CBM-A′ (a derivative of intermediate CBM-A of Scheme 1) under basic conditions and with a reducing agent to afford compounds LDD-c, which are compounds of Formula (I).

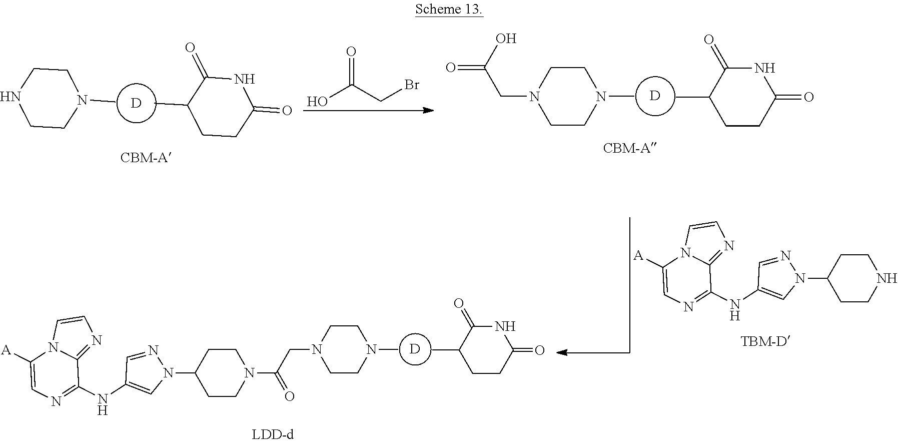

wherein A and Ring D are as described for Formula (I).

Scheme 13 illustrates an approach to the synthesis of compounds LDD-d. CBM-A′ (a derivative of intermediate CBM-A of Scheme 1) and 2-bromoacetic acid can be coupled under basic conditions to yield CBM-A″. CBM-A″ is then coupled with TBM-D′ (a derivative of intermediate TBM-D of Scheme 4) under basic conditions to afford compounds LDD-d, which are compounds of Formula (I).

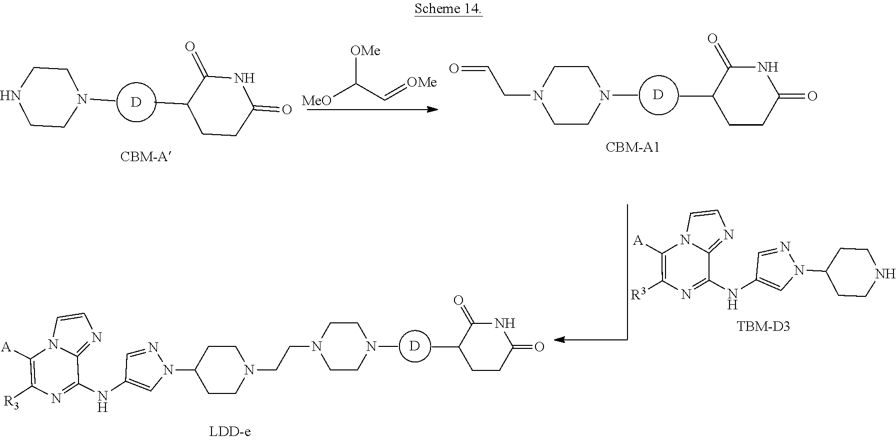

wherein A, R³, and Ring D are as described for Formula (I).

Scheme 14 illustrates an approach to the synthesis of compounds LDD-e. CBM-A′ (a derivative of intermediate CBM-A of Scheme 1) and 2,2-dimethoxyacetaldehyde can be coupled in the presence of a reducing agent, followed by reaction with strong acid to yield CBM-A1. CBM-A1 is then coupled with TBM-D3 (a derivative of intermediate TBM-D of Scheme 4) in the presence of a reducing agent to afford compounds LDD-e, which are compounds of Formula (I).

### Methods of Use

Embodiments of the present disclosure provide a method for modulating IRAK3 in a subject in need thereof, the method comprising administering to the subject an effective amount of a compound of Formula (I). Modulation (e.g., inhibition or activation) of IRAK3 can be assessed and demonstrated by a wide variety of ways known in the art. Kits and commercially available assays can be utilized for determining whether and to what degree IRAK3 has been modulated (e.g., inhibited or activated).

In one aspect, provided herein is a method of modulating IRAK3 comprising contacting IRAK3 with an effective amount of a compound of Formula (I) or any embodiment or variation thereof. In some embodiments, the compound of Formula (I) inhibits IRAK3. In other embodiments, the compound of Formula (I) activates IRAK3. In some embodiments, the compound of Formula (I) causes degradation of IRAK3.

In some embodiments, provided herein is a method for targeting IRAK3 for degradation comprising contacting IRAK3 with an effective amount of a compound of Formula (I) or any embodiment or variation thereof.

In some embodiments, a compound of Formula (I) modulates the activity of IRAK3 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) modulates the activity of IRAK3 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

Also provided in certain embodiments of the present disclosure is a method for degrading IRAK3 in a subject in need thereof, the method comprising administering to the subject an effective amount of a compound of Formula (I). Degradation of IRAK3 can be assessed and demonstrated by a wide variety of ways known in the art. Kits and commercially available assays, including cell-based assays, can be utilized for determining whether and to what degree IRAK3 has been degraded.

In one aspect, provided herein is a method of degrading IRAK3 comprising contacting IRAK3 with an effective amount of a compound of Formula (I) or any embodiment or variation thereof. In some embodiments, the compound of Formula (I) partially degrades IRAK3. In some embodiments, the compound of Formula (I) fully degrades IRAK3.

In some embodiments, a compound of Formula (I) degrades IRAK3 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) degrades IRAK3 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating cancer in a subject in need thereof, comprising administering to the subject an effective amount of a compound of Formula (I). In some embodiments, provided herein is a method for preventing cancer in a subject in need thereof, comprising administering to the subject an effective amount of a compound of Formula (I). Non-limiting examples of cancer include bladder cancer, breast cancer, esophageal cancer, colon cancer, head and neck cancer, kidney cancer, lung cancer, pancreatic cancer, prostate cancer, melanoma, and gastric cancer.

In some embodiments, administering a compound of Formula (I) to a subject that is predisposed to cancer prevents the subject from developing any symptoms of the cancer (such as tumor growth or metastasis). In some embodiments, administering a compound of Formula (I) to a subject that does not yet display symptoms of cancer prevents the subject from developing any symptoms of the cancer. In some embodiments, administering a compound of Formula (I) to a subject in need thereof diminishes the extent of the cancer in the subject. In some embodiments, administering a compound of Formula (I) to a subject in need thereof stabilizes the cancer (prevents or delays the worsening of the cancer). In some embodiments, administering a compound of Formula (I) to a subject in need thereof delays the occurrence or recurrence of the cancer. In some embodiments, administering a compound of Formula (I) to a subject in need thereof slows the progression of the cancer. In some embodiments, administering a compound of Formula (I) to a subject in need thereof provides a partial remission of the cancer. In some embodiments, administering a compound of Formula (I) to a subject in need thereof provides a total remission of the cancer. In some embodiments, administering a compound of Formula (I) to a subject in need thereof decreases the dose of one or more other medications required to treat the cancer. In some embodiments, administering a compound of Formula (I) to a subject in need thereof enhances the effect of another medication used to treat the cancer. In some embodiments, administering a compound of Formula (I) to a subject in need thereof delays the progression of the cancer. In some embodiments, administering a compound of Formula (I) to a subject in need thereof increases the quality of life of the subject having cancer. In some embodiments, administering a compound of Formula (I) to a subject in need thereof prolongs survival of a subject having cancer.

In one aspect, provided herein is method of preventing a subject that is predisposed to cancer from developing cancer, the method comprising administering a compound of Formula (I) to the subject.

In some aspects, provided herein is a method of diminishing the extent of cancer in a subject, the method comprising administering a compound of Formula (I) to the subject. In some embodiments, provided herein is a method of stabilizing cancer in a subject, the method comprising administering a compound of Formula (I) to the subject. In some embodiments, the method prevents the worsening of the cancer.

In another aspect, provided herein is a method of delaying the occurrence or recurrence of cancer in a subject, the method comprising administering a compound of Formula (I) to the subject.

In some embodiments, provided herein is a method of slowing the progression of cancer in a subject, the method comprising administering a compound of Formula (I) to the subject. In some embodiments, the method provides a partial remission of the cancer. In some embodiments, the method provides a total remission of the cancer.

In further aspects, provided herein is a method of decreasing the dose of one or more other medications required to treat cancer in a subject, the method comprising administering a compound of Formula (I) to the subject. In some embodiments, provided herein is a method of enhancing the effect of another medication used to treat cancer in a subject, the method comprising administering a compound of Formula (I) to the subject.

Also provided here is a method of delaying the progression of cancer in a subject, the method comprising administering a compound of Formula (I) to the subject. In some embodiments, the method increases the quality of life of the subject having cancer. In some embodiments, the method prolongs survival of the subject having cancer.

In some embodiments, compounds of Formula (I) are useful for treating a cancer selected from bladder cancer, breast cancer, esophageal cancer, colon cancer, head and neck cancer, kidney cancer, lung cancer, pancreatic cancer, prostate cancer, melanoma, and gastric cancer.

In some embodiments, provided herein is a method to enhance immunity in a subject receiving a vaccine, comprising administering to the subject an effective amount of a compound of Formula (I). In some embodiments, the compound of Formula (I) is administered to the subject prior to the administration of a vaccine. In some embodiments, the compound of Formula (I) is administered to the subject simultaneously to the administration of a vaccine. In some embodiments, the compound of Formula (I) is administered to the subject following the administration of a vaccine. In some embodiments, the compound of Formula (I) is formulated as a component of the vaccine. In some embodiments, the compound of Formula (I) is formulated separately from the vaccine.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of Formula (I) administered will depend on such factors as the solubility of the active component, the formulation used and the route of administration.

In some embodiments, a compound of Formula (I) is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (I).

In a particular embodiment, provided herein are unit dosage formulations comprising about 0.1 mg or 100 mg of a compound of Formula (I).

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a compound of Formula (I).

A compound of Formula (I) can be administered once, twice, three, four or more times daily. In a particular embodiment, doses of 100 mg or less are administered as a once daily dose and doses of more than 100 mg are administered twice daily in an amount equal to one half of the total daily dose.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### Exemplary Embodiments

The present disclosure is further described by the following embodiments.

Embodiment 1. A compound of Formula (I):

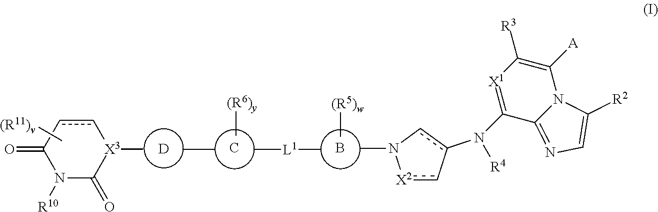

or a pharmaceutically acceptable salt thereof, wherein:
A is C₁-C₆ alkyl, phenyl, C₃-C₆ cycloalkyl, 5- to 6-membered heteroaryl, or 6- to 10-membered heterocyclyl, wherein the phenyl, cycloalkyl, heteroaryl, and heterocyclyl are substituted by x R¹ groups, and wherein the heteroaryl and heterocyclyl contain 1-3 heteroatoms selected from N and O;
each R¹ is independently halo, C₁-C₆ alkyl, C₃-C₆ cycloalkyl, C₁-C₆ alkoxy, C₁-C₆ haloalkyl, or —SO₂(C₁-C₆ alkyl);
or two R¹ groups on adjacent carbon atoms are taken together to form a fused C₃-C₆ cycloalkyl or a fused

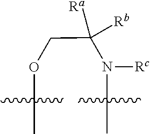

group;
R^{a} and R^{b} are each H or are taken together to form an oxo group;
R^{c} is H or C₁-C₆ alkyl;
x is 0-5;
R² is H or C₁-C₆ alkyl;
R³ is H or C₁-C₆ alkyl;
R⁴ is H or C₁-C₆ alkyl;
X¹ is CH or N;
X² is N or CH₂;
Ring B is C₃-C₆ cycloalkylene or 5- to 7-membered heterocyclylene containing 1 or 2 nitrogen atoms;
each R⁵ is independently halo, C₁-C₆ alkyl, or C₁-C₆ haloalkyl;
w is 0-5;
L¹ is —C(O)(CH₂)ₙ—, —(CH₂)ₙ—, or —(CH₂)ₙC(O)—;
n is 1-6;
Ring C is 5- to 10-membered heterocyclylene containing 1 or 2 nitrogen atoms;
each R⁶ is independently halo, C₁-C₆ haloalkyl, or C₁-C₆ alkyl;
y is 0-5;
Ring D is

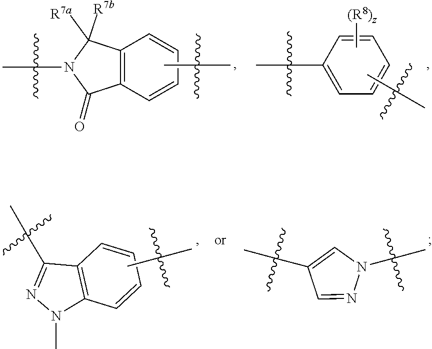

R^{7a} and R^{7b} are each H or are taken together to form an oxo group;
each R⁸ is independently halo, C₁-C₆ alkyl, C₁-C₆ haloalkyl, or C₁-C₆ alkoxy;
z is 0-4;
X³ is N or CR⁹;
R⁹ is H or C₁-C₆ alkyl;
R¹⁰ is H or C₁-C₆ alkyl;
each R¹¹ is independently halo, C₁-C₆ alkyl, or C₁-C₆ haloalkyl;
v is 0-4; and
each [symbol] is independently a single bond or double bond.

Embodiment 2. The compound of embodiment 1, or a pharmaceutically acceptable salt thereof, wherein:
A is C₁-C₃ alkyl, phenyl, C₃-C₅ cycloalkyl, 5- to 6-membered heteroaryl, or 8- to 10-membered heterocyclyl, wherein the phenyl, cycloalkyl, heteroaryl, and heterocyclyl are substituted by x R¹ groups.

Embodiment 3. The compound of embodiment 1 or 2, or a pharmaceutically acceptable salt thereof, wherein:
x is 0-3.

Embodiment 4. The compound of any one of embodiments 1-3, or a pharmaceutically acceptable salt thereof, wherein:
each R¹ is independently halo, C₁-C₃ alkyl, C₃-C₅ cycloalkyl, C₁-C₃ alkoxy, C₁-C₃ haloalkyl, or —SO₂(C₁-C₃ alkyl);
or two R¹ groups on adjacent carbon atoms are taken together to form a fused C₃-C₅ cycloalkyl or a fused

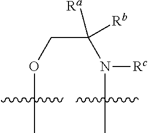

group;
R^{a} and R^{b} are each H or are taken together to form an oxo group; and
R^{c} is H or C₁-C₃ alkyl.

Embodiment 5. The compound of embodiment 4, or a pharmaceutically acceptable salt thereof, wherein:
each R¹ is independently F, —CH₃, cyclopropyl, —OCH₃, or —SO₂(CH₃);
or two R¹ groups on adjacent carbon atoms are taken together to form a fused cyclopropyl, cyclobutyl,

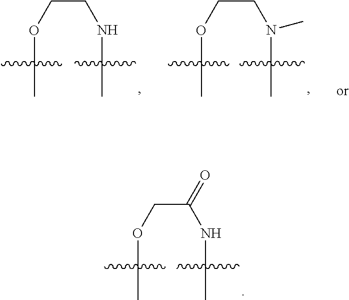

Embodiment 6. The compound of any one of embodiments 1-5, or a pharmaceutically acceptable salt thereof, wherein:
A is

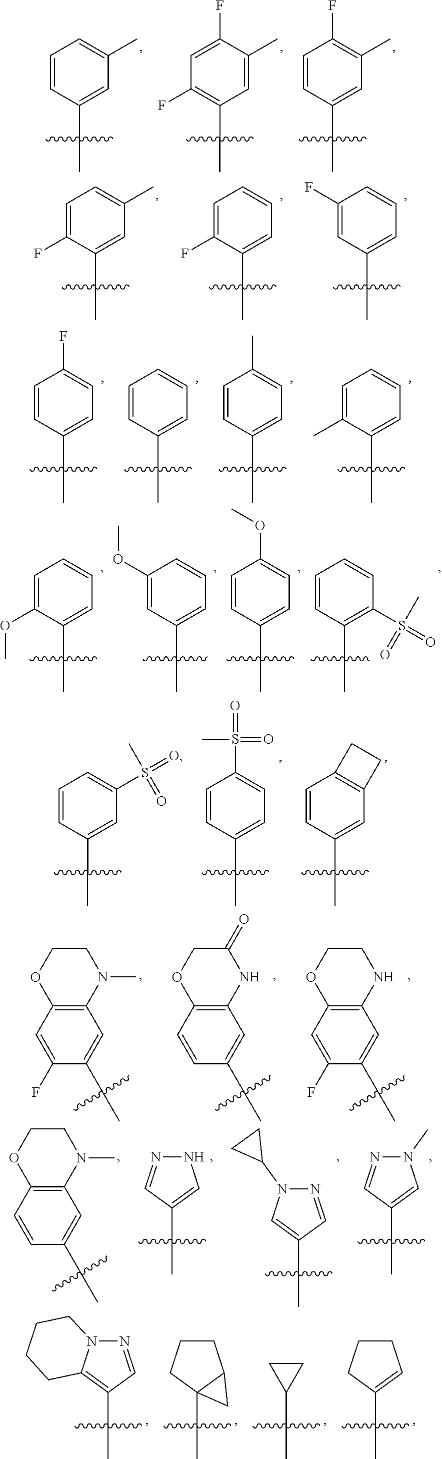

—CH₃, or —CH₂CH₃.

Embodiment 7. The compound of any one of embodiments 1-6, or a pharmaceutically acceptable salt thereof, wherein:
R² is H or C₁-C₃ alkyl.

Embodiment 8. The compound of embodiment 7, or a pharmaceutically acceptable salt thereof, wherein:
R² is H or —CH₃.

Embodiment 9. The compound of any one of embodiments 1-8, or a pharmaceutically acceptable salt thereof, wherein:
R³ is H or C₁-C₃ alkyl.

Embodiment 10. The compound of embodiment 9, or a pharmaceutically acceptable salt thereof, wherein:
R³ is H or —CH₃.

Embodiment 11. The compound of any one of embodiments 1-10, or a pharmaceutically acceptable salt thereof, wherein:
R⁴ is H or C₁-C₃ alkyl.

Embodiment 12. The compound of embodiment 11, or a pharmaceutically acceptable salt thereof, wherein:
R⁴ is H or —CH₃.

Embodiment 13. The compound of any one of embodiments 1-12, or a pharmaceutically acceptable salt thereof, wherein:
X¹ is CH.

Embodiment 14. The compound of any one of embodiments 1-12, or a pharmaceutically acceptable salt thereof, wherein:
X¹ is N.

Embodiment 15. The compound of any one of embodiments 1-14, or a pharmaceutically acceptable salt thereof, wherein:
X² is N.

Embodiment 16. The compound of any one of embodiments 1-14, or a pharmaceutically acceptable salt thereof, wherein:
X² is CH₂.

Embodiment 17. The compound of any one of embodiments 1-16, or a pharmaceutically acceptable salt thereof, wherein:

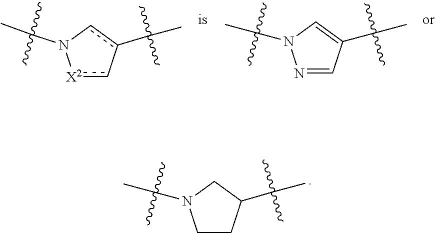

Embodiment 18. The compound of any one of embodiments 1-17, or a pharmaceutically acceptable salt thereof, wherein:
Ring B is C₄-C₆ cycloalkylene or 6- to 7-membered heterocyclylene containing one nitrogen atom.

Embodiment 19. The compound of embodiment 18, or a pharmaceutically acceptable salt thereof, wherein:
Ring B is

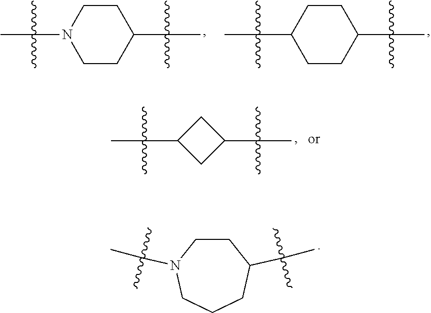 [SMILES: *C1CCN(*)CC1 ; *C1CCC(*)CC1 ; *C1CC(*)C1 ; *C1CCCN(*)CC1]

Embodiment 20. The compound of any one of embodiments 1-19, or a pharmaceutically acceptable salt thereof, wherein:
w is 0-2.

Embodiment 21. The compound of any one of embodiments 1-20, or a pharmaceutically acceptable salt thereof, wherein:
each R⁵ is independently halo, C₁-C₃ alkyl, or C₁-C₃ haloalkyl.

Embodiment 22. The compound of embodiment 21, or a pharmaceutically acceptable salt thereof, wherein:
each R⁵ is independently F, —CF₃, or —CH₃.

Embodiment 23. The compound of any one of embodiments 1-22, or a pharmaceutically acceptable salt thereof, wherein:

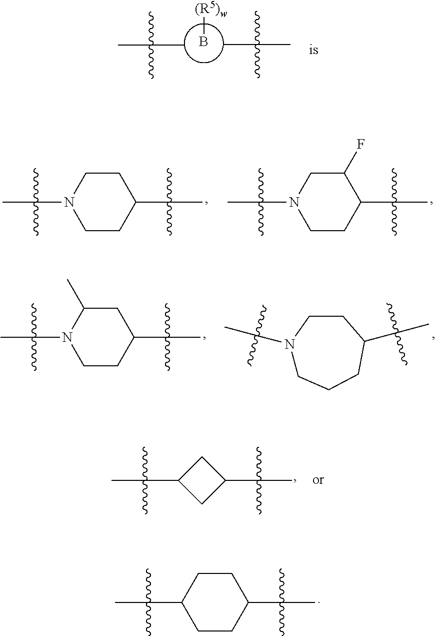

Embodiment 24. The compound of any one of embodiments 1-23, or a pharmaceutically acceptable salt thereof, wherein:
L¹ is —C(O)CH₂—, —(CH₂)ₙ—, or —CH₂C(O)—; and
n is 1-5.

Embodiment 25. The compound of embodiment 24, or a pharmaceutically acceptable salt thereof, wherein:
L¹ is —C(O)CH₂—, —CH₂—, —CH₂CH₂—, —CH₂CH₂CH₂—, —CH₂CH₂CH₂CH₂—, or —CH₂C(O)—.

Embodiment 26. The compound of any one of embodiments 1-25, or a pharmaceutically acceptable salt thereof, wherein:
Ring C is 6- to 8-membered heterocyclylene containing 1 or 2 nitrogen atoms.

Embodiment 27. The compound of embodiment 26, or a pharmaceutically acceptable salt thereof, wherein:
Ring C is

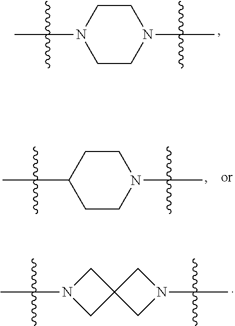 [SMILES: *N1CCN(*)CC1 ; *C1CCN(*)CC1 ; *N1CC2(C1)CN(*)C2]

Embodiment 28. The compound of any one of embodiments 1-27, or a pharmaceutically acceptable salt thereof, wherein:
y is 0-3.

Embodiment 29. The compound of any one of embodiments 1-28, or a pharmaceutically acceptable salt thereof, wherein:
each R⁶ is independently halo, C₁-C₃ haloalkyl, or C₁-C₃ alkyl.

Embodiment 30. The compound of embodiment 29, or a pharmaceutically acceptable salt thereof, wherein:
each R⁶ is independently Cl, —CF₃, or —CH₃.

Embodiment 31. The compound of any one of embodiments 1-30, or a pharmaceutically acceptable salt thereof, wherein:

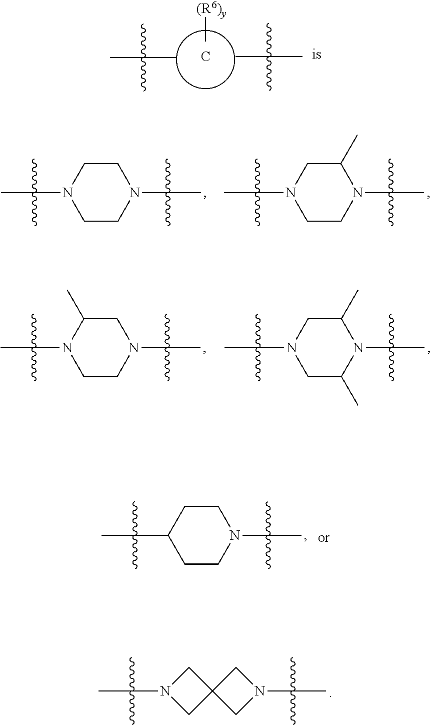

Embodiment 32. The compound of any one of embodiments 1-31, or a pharmaceutically acceptable salt thereof, wherein:
Ring D is

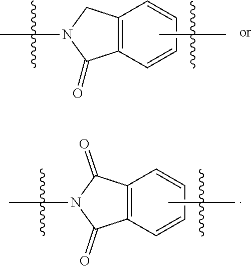

Embodiment 33. The compound of any one of embodiments 1-31, or a pharmaceutically acceptable salt thereof, wherein:
Ring D is

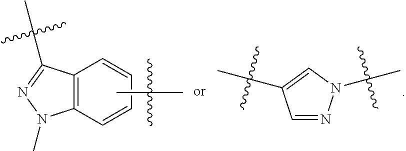

Embodiment 34. The compound of any one of embodiments 1-31, or a pharmaceutically acceptable salt thereof, wherein:
Ring D is

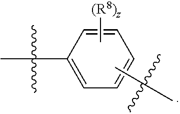

Embodiment 35. The compound of embodiment 34, or a pharmaceutically acceptable salt thereof, wherein:
z is 0-2.

Embodiment 36. The compound of embodiment 34 or 35, or a pharmaceutically acceptable salt thereof, wherein:
each R⁸ is independently halo, C₁-C₃ alkyl, C₁-C₃ haloalkyl, or C₁-C₃ alkoxy.

Embodiment 37. The compound of embodiment 36, or a pharmaceutically acceptable salt thereof, wherein:
each R⁸ is independently F, Cl, —CH₃, —OCH₃, or —CF₃.

Embodiment 38. The compound of any one of embodiments 1-37, or a pharmaceutically acceptable salt thereof, wherein:
Ring D is

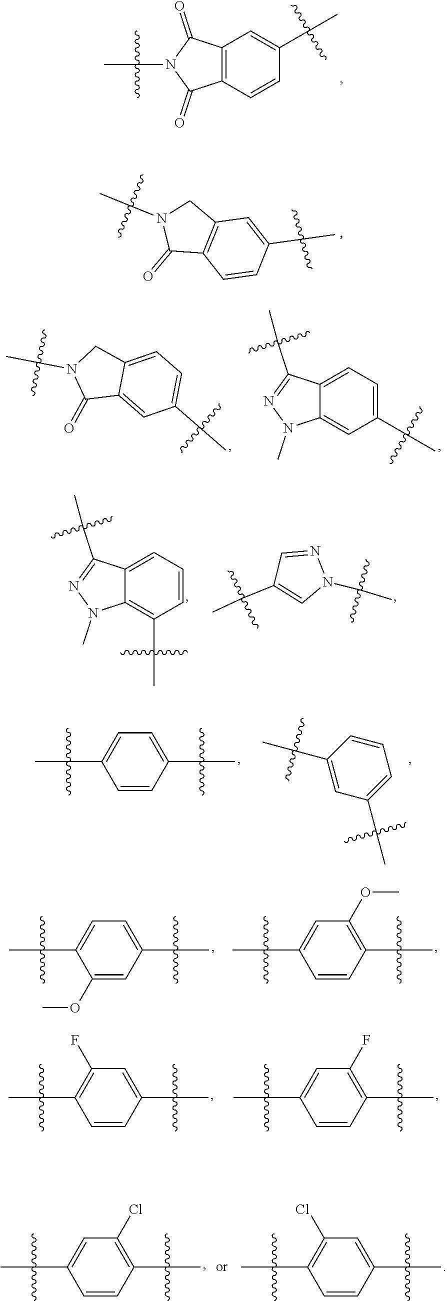

Embodiment 39. The compound of any one of embodiments 1-38, or a pharmaceutically acceptable salt thereof, wherein:
X³ is N.

Embodiment 40. The compound of any one of embodiments 1-38, or a pharmaceutically acceptable salt thereof, wherein:
X³ is CR⁹; and
R⁹ is H or C₁-C₃ alkyl.

Embodiment 41. The compound of embodiment 40, or a pharmaceutically acceptable salt thereof, wherein:
X³ is CR⁹; and
R⁹ is H or —CH₃.

Embodiment 42. The compound of any one of embodiments 1-41, or a pharmaceutically acceptable salt thereof, wherein:
R¹⁰ is H or C₁-C₃ alkyl.

Embodiment 43. The compound of embodiment 42, or a pharmaceutically acceptable salt thereof, wherein:
R¹⁰ is H or —CH₃.

Embodiment 44. The compound of any one of embodiments 1-43, or a pharmaceutically acceptable salt thereof, wherein:
v is 0-2.

Embodiment 45. The compound of any one of embodiments 1-44, or a pharmaceutically acceptable salt thereof, wherein:
each R¹¹ is independently halo, C₁-C₃ alkyl, or C₁-C₃ haloalkyl.

Embodiment 46. The compound of embodiment 45, or a pharmaceutically acceptable salt thereof, wherein:
each R¹¹ is independently F, —CH₃, or —CF₃.

Embodiment 47. The compound of any one of embodiments 1-46, or a pharmaceutically acceptable salt thereof, wherein:

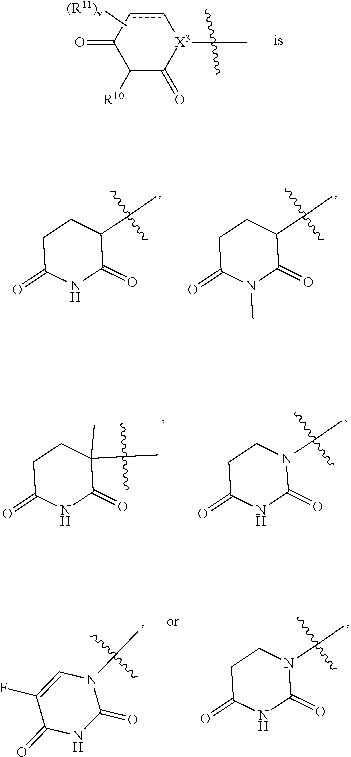

Embodiment 48. The compound of any one of embodiments 1-12, 14, 15, 17-38, and 40-47, or a pharmaceutically acceptable salt thereof, wherein the compound is of Formula (II):

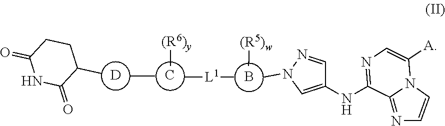

Embodiment 49. The compound of any one of embodiments 1-12, 14, 15, 17-32, 34-38, and 40-47, or a pharmaceutically acceptable salt thereof, wherein the compound is of Formula (IIIa), (IIIb), or (IIIc):

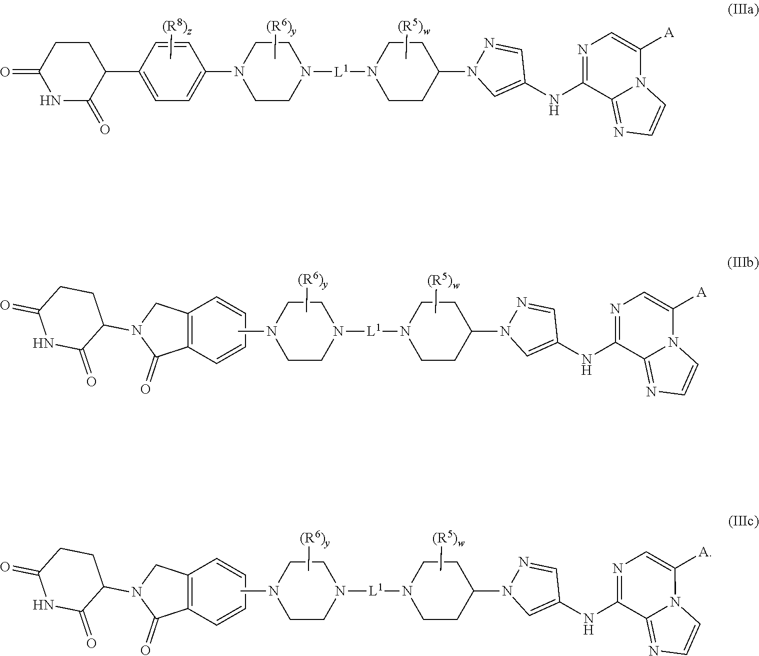

Embodiment 50. The compound of any one of embodiments 1-12, 14, 15, 17-31, 34-38, and 40-47, or a pharmaceutically acceptable salt thereof, wherein the compound is of Formula (IVa) or (IVb):

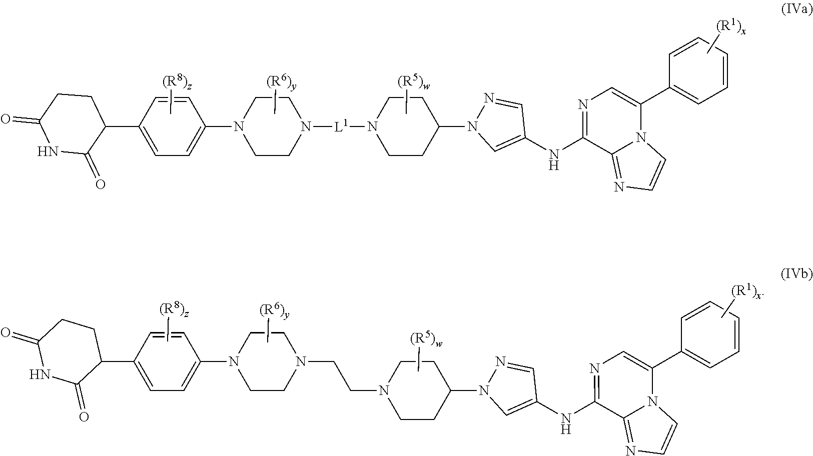

Embodiment 51. A compound selected from the compounds of Table 1 or a pharmaceutically acceptable salt thereof.

Embodiment 52. A pharmaceutical composition comprising the compound of any one of embodiments 1-51, or a pharmaceutically acceptable salt thereof, and a pharmaceutically acceptable excipient.

Embodiment 53. A method of modulating Interleukin-1 Receptor-Associated Kinase 3 (IRAK3) comprising contacting IRAK3 with an effective amount of the compound of any one of embodiments 1-51, or a pharmaceutically acceptable salt thereof, or the pharmaceutical composition of embodiment 52.

Embodiment 54. A method of treating cancer in a subject in need thereof, comprising administering to the subject an effective amount of the compound of any one of embodiments 1-51, or a pharmaceutically acceptable salt thereof, or the pharmaceutical composition of embodiment 52.

Embodiment 55. The method of embodiment 54, wherein the cancer is selected from bladder cancer, breast cancer, esophgeal cancer, colon cancer, head and neck cancer, kidney cancer, lung cancer, pancreatic cancer, prostate cancer, melanoma, and gastric cancer.

Embodiment 56. A method of enhancing immunity in a subject receiving a vaccine, comprising administering to the subject an effective amount of the compound of any one of embodiments 1-51, or a pharmaceutically acceptable salt thereof, or the pharmaceutical composition of embodiment 52.

Embodiment 57. The method of embodiment 56, wherein the subject is administered the vaccine prior to, concurrently with, or after administration of the compound of any one of embodiments 1-51, or a pharmaceutically acceptable salt thereof, or the pharmaceutical composition of embodiment 52.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

Abbreviations

| | |
|---|---|
| 2-MeTHF | 2-methyltetrahydrofuran |
| AcOH | acetic acid |
| aq. | aqueous |
| atm | atmospheric pressure |
| BINAP | (2,2′-bis(diphenylphosphino)-1,1′-binaphthyl) |
| Boc | tert-butyloxycarbonyl |
| BTEAC | benzyltriethylammonium chloride |
| BTEAC | benzyltriethylammonium chloride |
| ca. | circa |
| Cmp | Compound |
| DCE | dichloroethane |
| DCM | dichloromethane |
| DIAD | diisopropyl azodicarboxylate |
| DIBAL-H | diisobutylaluminium hydride |
| DIPEA | diisopropylethylamine |
| DIPEA | N,N-diisopropylethylamine |
| DMA | dimethylacetamide |
| DMAP | 4-dimethylaminopyridine |
| DMAP | 4-dimethylaminopyridine |
| DMF | dimethyl formamide |
| DMPAO | 2-((2,6-dimethylphenyl)amino)-2-oxoacetic acid |
| DMPAO | 2-((2,6-dimethylphenyl)amino)-2-oxoacetic acid |
| DMSO | dimethyl sulfoxide |
| equiv. or equiv or eq. | equivalent |
| ESI | electrospray ionization |
| Et | ethyl |
| EtOAc | ethyl acetate |
| EtOH | ethanol |
| FA | formic acid |
| h | hour(s) |
| HPLC | high pressure liquid chromatography |
| IBX | 2-iodoxybenzoic acid |
| IPA | isopropyl alcohol |
| KOAc | potassium acetate |
| LCMS | liquid chromatography mass spectrometry |
| Me | methyl |
| MeCN | acetonitrile |
| MeOH | methanol |
| min | minute(s) |
| MS | mass spectrometry |
| MsCl | methanesulfonyl chloride |
| MTBE | methyl tert-butyl ether |
| NEt₃ | triethylamine |
| NMR | nuclear magnetic resonance |
| O.N. | overnight |
| OTBDPS | tert-butyldiphenylsilyl |
| PE | petroleum ether |
| Ph | phenyl |
| Pin | pinacolato |
| PyAOP | (7-azabenzotriazol-1-yloxy)tripyrrolidinophosphonium |
| | hexafluorophosphate |
| quant. | quantitative |
| rac | racemic mixture |
| Rf | retention factor |
| rt, r.t. | room temperature |
| sat. | saturated |
| SFC | supercritical fluid chromatography |
| TBAF | tetra-n-butylammonium fluoride |
| TFA | trifluoroacetic acid |
| THF | tetrahydrofuran |
| TLC | thin layer chromatography |
| TMSCHN₂ | trimethylsilyldiazomethane |

### Synthetic Examples

### Overview of the Procedures

The synthesized compounds were prepared from two main building blocks: the Target-Binding Moieties (TBMs) and the Cereblon-Binding Moieties (CBMs). The procedures for these building blocks can be found below in their respective sections. The TBMs and CBMs were then linked through 2 or 3 step procedures to form heterobifunctional molecules (i.e., Ligand-Directed Degraders, LDDs). Table 2 lists the building blocks utilized to synthesize each example and the sections where those procedures are located.

TABLE 2
Procedures for Synthesizing LDDs from CBMs and TBMs

| | TBM | TBM | CBM | LDD |
|---|---|---|---|---|
| Cmp | # | Proc. | # | Proc. |
| 1 | 1 | TBM-D | 1 | LDD-I |
| 2 | 1 | TBM-D | 1 | LDD-J |
| 3 | 1 | TBM-D | 2 | LDD-J |
| 4 | 1 | TBM-D | 2 | LDD-J |
| 5 | 1 | TBM-D | 2 | LDD-J |
| 6 | 2 | TBM-D | 1 | LDD-I |
| 7 | 2 | TBM-D | 3 | LDD-J |
| 8 | 2 | TBM-D | 2 | LDD-J |
| 9 | 2 | TBM-D | 4 | LDD-J |
| 10 | 2 | TBM-D | 5 | LDD-I |
| 11 | 43 | TBM-F | 2 | LDD-L |
| 12 | 3 | TBM-D | 2 | LDD-J |
| 13 | 2 | TBM-D | 6 | LDD-J |
| 14 | 44 | TBM-F | 2 | LDD-L |
| 15 | 4 | TBM-D | 2 | LDD-J |
| 16 | 1 | TBM-D | 7 | LDD-I |
| 17 | 2 | TBM-D | 2 | LDD-I |
| 18 | 2 | TBM-D | 7 | LDD-I |
| 19 | 1 | TBM-D | 5 | LDD-I |
| 20 | 1 | TBM-D | 2 | LDD-I |
| 21 | 4 | TBM-D | 1 | LDD-I |
| 22 | 4 | TBM-D | 2 | LDD-I |
| 23 | 2 | TBM-D | 2 | LDD-M |
| 24 | 2 | TBM-D | 1 | LDD-M |
| 25 | 2 | TBM-D | 8 | LDD-J |
| 26 | 5 | TBM-D | 2 | LDD-J |
| 27 | 44 | TBM-F | 3 | LDD-L |
| 28 | 44 | TBM-F | 4 | LDD-L |
| 29 | 6 | TBM-D | 4 | LDD-J |
| 30 | 6 | TBM-D | 2 | LDD-J |
| 31 | 2 | TBM-D | 9 | LDD-J |
| 32 | 7 | TBM-D | 4 | LDD-J |
| 33 | 8 | TBM-D | 2 | LDD-J |
| 34 | 5 | TBM-D | 3 | LDD-J |
| 35 | 5 | TBM-D | 4 | LDD-J |
| 36 | 9 | TBM-D | 2 | LDD-J |
| 37 | 43 | TBM-F | 4 | LDD-L |
| 38 | 7 | TBM-D | 3 | LDD-J |
| 39 | 2 | TBM-D | 10 | LDD-J |
| 40 | 45 | TBM-F | 2 | LDD-L |
| 41 | 2 | TBM-D | 11 | LDD-J |
| 42 | 2 | TBM-D | 12 | LDD-J |
| 43 | 2 | TBM-D | 13 | LDD-J |
| 44 | 2 | TBM-D | 14 | LDD-J |
| 45 | 46 | TBM-F | 2 | LDD-L |
| 46 | 7 | TBM-D | 2 | LDD-J |
| 47 | 47 | TBM-F | 2 | LDD-L |
| 48 | 10 | TBM-D | 2 | LDD-J |
| 49 | 2 | TBM-D | 15 | LDD-J |
| 50 | 2 | TBM-D | 16 | LDD-J |
| 51 | 11 | TBM-D | 2 | LDD-J |
| 52 | 60 | singleton | 2 | LDD-J |
| 53 | 2 | TBM-D | 17 | LDD-J |
| 54 | 2 | TBM-D | 18 | LDD-J |
| 55 | 2 | TBM-D | 19 | LDD-J |
| 56 | 2 | TBM-D | 20 | LDD-J |
| 57 | 43 | TBM-F | 3 | LDD-L |
| 58 | 2 | TBM-D | 21 | LDD-J |
| 59 | 6 | TBM-D | 3 | LDD-J |
| 60 | 12 | TBM-D | 2 | LDD-J |
| 61 | 13 | TBM-D | 2 | LDD-J |
| 62 | 14 | TBM-D | 2 | LDD-J |
| 63 | 15 | TBM-D | 2 | LDD-J |
| 64 | 48 | TBM-F | 2 | LDD-L |
| 65 | 16 | TBM-D | 2 | LDD-J |
| 66 | 51 | TBM-G | 2 | LDD-L |
| 67 | 52 | TBM-G | 2 | LDD-L |
| 68 | 17 | TBM-D | 2 | LDD-J |
| 69 | 18 | TBM-D | 2 | LDD-J |
| 70 | 28 | TBM-E | 2 | LDD-K |
| 71 | 53 | TBM-G | 2 | LDD-L |
| 72 | 19 | TBM-D | 2 | LDD-J |
| 73 | 20 | TBM-D | 2 | LDD-J |
| 74 | 29 | TBM-E | 2 | LDD-K |
| 75 | 30 | TBM-E | 2 | LDD-K |
| 76 | 2 | TBM-D | 22 | LDD-J |
| 77 | 2 | TBM-D | 23 | LDD-J |
| 78 | 31 | TBM-E | 2 | LDD-K |
| 79 | 32 | TBM-E | 2 | LDD-K |
| 80 | 33 | TBM-E | 2 | LDD-K |
| 81 | 43 | TBM-F | 23 | LDD-L |
| 82 | 43 | TBM-F | 22 | LDD-L |
| 83 | 34 | TBM-E | 2 | LDD-K |
| 84 | 21 | TBM-D | 2 | LDD-O |
| 85 | 35 | TBM-E | 2 | LDD-K |
| 86 | 36 | TBM-E | 2 | LDD-K |
| 87 | 37 | TBM-E | 2 | LDD-K |
| 88 | 38 | TBM-E | 2 | LDD-K |
| 89 | 39 | TBM-D | 2 | LDD-O |
| 90 | 22 | TBM-D | 2 | LDD-O |
| 91 | 49 | TBM-F | 2 | LDD-L |
| 92 | 40 | TBM-E | 2 | LDD-K |
| 93 | 41 | TBM-E | 2 | LDD-K |
| 94 | 42 | TBM-E | 2 | LDD-K |
| 95 | 43 | TBM-F | 24 | LDD-L |
| 96 | 54 | TBM-G | 2 | LDD-N |
| 97 | 2 | TBM-D | 25 | LDD-J |
| 98 | 23 | TBM-D | 2 | LDD-J |
| 99 | 55 | TBM-G | 2 | LDD-N |
| 100 | 56 | TBM-H | 2 | LDD-L |
| 101 | 44 | TBM-F | 25 | LDD-L |
| 102 | 2 | TBM-D | 26 | LDD-J |
| 103 | 50 | TBM-F | 2 | LDD-L |
| 104 | 43 | TBM-F | 25 | LDD-L |
| 105 | 43 | TBM-F | 10 | LDD-L |
| 106 | 43 | TBM-F | 27 | LDD-L |
| 107 | 43 | TBM-F | 28 | LDD-L |
| 108 | 59 | singleton | 2 | LDD-L |
| 109 | 2 | TBM-D | 29 | LDD-J |
| 110 | 2 | TBM-D | 30 | LDD-J |
| 111 | 24 | TBM-D | 2 | LDD-O |
| 112 | 25 | TBM-D | 2 | LDD-O |
| 113 | 7 | TBM-D | 11 | LDD-J |
| 114 | 26 | TBM-D | 2 | LDD-O |
| 115 | 7 | TBM-D | 10 | LDD-J |
| 116 | 2 | TBM-D | 31 | LDD-J |
| 117 | 43 | TBM-F | 11 | LDD-L |
| 118 | 57 | TBM-H | 2 | LDD-L |
| 119 | 27 | TBM-D | 2 | LDD-J |
| 120 | 58 | TBM-H | 2 | LDD-L |

### Procedures for Cereblon-binding Moieties (CBMs)

Table 3 lists the general procedure utilized to synthesize each CBM molecule.

TABLE 3
General Procedures for the CBMs

| CBM # | Structure | Reference/General Procedure |
|---|---|---|
| CBM-1 | 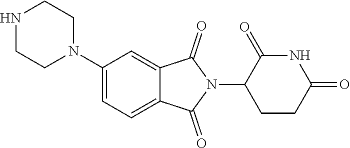 [SMILES: O=C1CCC(N2C(=O)c3ccc(N4CCNCC4)cc3C2=O)C(=O)N1] | Previously synthesized: J. Med. Chem. 2021, 74, 12831-12854 |
| CBM-2 | 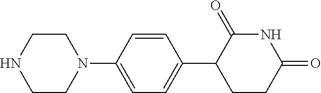 [SMILES: O=C1CCC(c2ccc(N3CCNCC3)cc2)C(=O)N1] | General Procedure: CBM-A |
| CBM-3 | 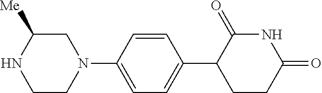 [SMILES: C[C@H]1CN(c2ccc(C3CCC(=O)NC3=O)cc2)CCN1] | General Procedure: CBM-B |
| CBM-4 | 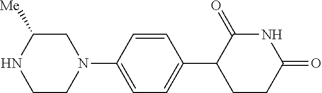 [SMILES: C[C@@H]1CN(c2ccc(C3CCC(=O)NC3=O)cc2)CCN1] | General Procedure: CBM-B |
| CBM-5 | 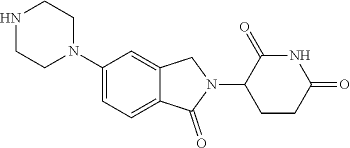 [SMILES: O=C1CCC(N2Cc3cc(N4CCNCC4)ccc3C2=O)C(=O)N1] | Procedure provided below |
| CBM-6 | 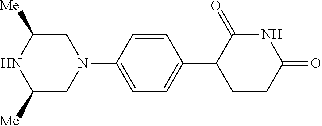 [SMILES: C[C@@H]1CN(c2ccc(C3CCC(=O)NC3=O)cc2)C[C@H](C)N1] | General Procedure: CBM-B |
| CBM-7 | 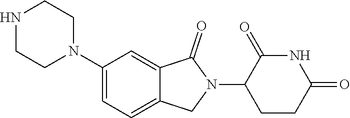 [SMILES: O=C1CCC(N2Cc3ccc(N4CCNCC4)cc3C2=O)C(=O)N1] | Procedure provided below |
| CBM-8 | 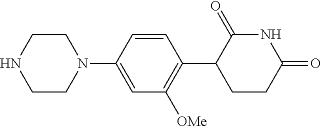 [SMILES: COc1cc(N2CCNCC2)ccc1C1CCC(=O)NC1=O] | General Procedure: CBM-B |
| CBM-9 | 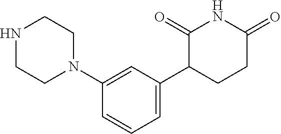 [SMILES: O=C1CCC(c2cccc(N3CCNCC3)c2)C(=O)N1] | Previously synthesized: WO 2020/051564 A1 |
| CBM-10 | 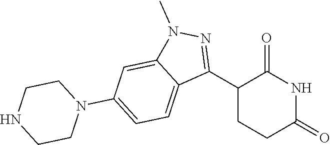 [SMILES: Cn1nc(C2CCC(=O)NC2=O)c2ccc(N3CCNCC3)cc21] | Procedure provided below |
| CBM-11 | 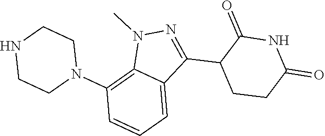 [SMILES: Cn1nc(C2CCC(=O)NC2=O)c2cccc(N3CCNCC3)c21] | Procedure provided below |
| CBM-12 | 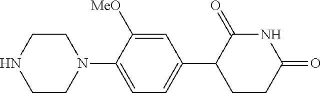 [SMILES: COc1cc(C2CCC(=O)NC2=O)ccc1N1CCNCC1] | General Procedure: CBM-A |
| CBM-13 | 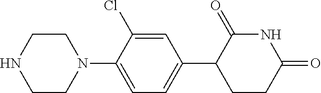 [SMILES: O=C1CCC(c2ccc(N3CCNCC3)c(Cl)c2)C(=O)N1] | General Procedure: CBM-B |
| CBM-14 | 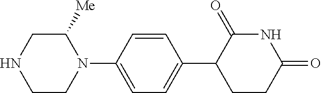 [SMILES: C[C@H]1CNCCN1c1ccc(C2CCC(=O)NC2=O)cc1] | General Procedure: CBM-B |
| CBM-15 | 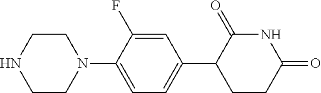 [SMILES: O=C1CCC(c2ccc(N3CCNCC3)c(F)c2)C(=O)N1] | General Procedure: CBM-B |
| CBM-16 | 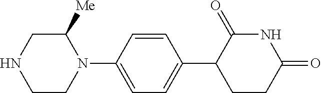 [SMILES: C[C@@H]1CNCCN1c1ccc(C2CCC(=O)NC2=O)cc1] | General Procedure: CBM-B |
| CBM-17 | 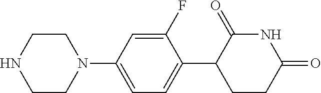 [SMILES: O=C1CCC(c2ccc(N3CCNCC3)cc2F)C(=O)N1] | General Procedure: CBM-B |
| CBM-18 | 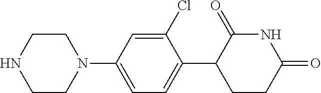 [SMILES: O=C1CCC(c2ccc(N3CCNCC3)cc2Cl)C(=O)N1] | General Procedure: CBM-B |
| CBM-19 | 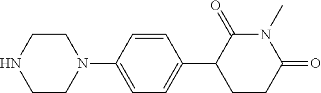 [SMILES: CN1C(=O)CCC(c2ccc(N3CCNCC3)cc2)C1=O] | Procedure provided below |
| CBM-20 | 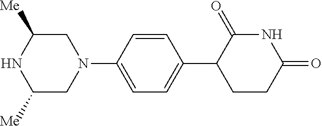 [SMILES: C[C@H]1CN(c2ccc(C3CCC(=O)NC3=O)cc2)C[C@H](C)N1] | General Procedure: CBM-B |
| CBM-21 | 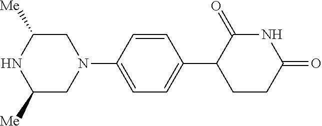 [SMILES: C[C@@H]1CN(c2ccc(C3CCC(=O)NC3=O)cc2)C[C@@H](C)N1] | General Procedure: CBM-B |
| CBM-22 | 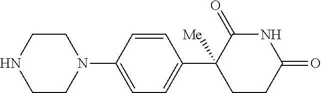 [SMILES: C[C@@]1(c2ccc(N3CCNCC3)cc2)CCC(=O)NC1=O] | General Procedure: CBM-C |
| CBM-23 | 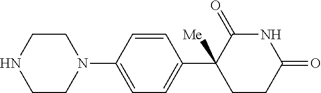 [SMILES: C[C@]1(c2ccc(N3CCNCC3)cc2)CCC(=O)NC1=O] | General Procedure: CBM-C |
| CBM-24 | 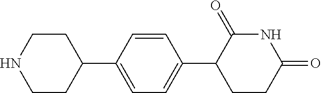 [SMILES: O=C1CCC(c2ccc(C3CCNCC3)cc2)C(=O)N1] | General Procedure: CBM-A |
| CBM-25 | 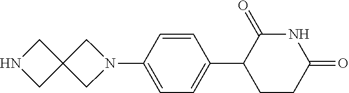 [SMILES: O=C1CCC(c2ccc(N3CC4(CNC4)C3)cc2)C(=O)N1] | Procedure provided below |
| CBM-26 | 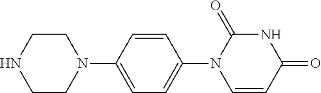 [SMILES: O=c1ccn(-c2ccc(N3CCNCC3)cc2)c(=O)[nH]1] | Procedure provided below |
| CBM-27 | 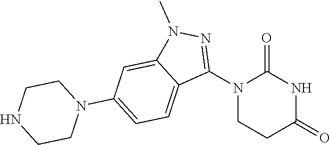 [SMILES: Cn1nc(N2CCC(=O)NC2=O)c2ccc(N3CCNCC3)cc21] | General Procedure: CBM-C |
| CBM-28 | 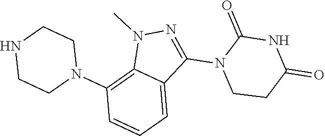 [SMILES: Cn1nc(N2CCC(=O)NC2=O)c2cccc(N3CCNCC3)c21] | General Procedure: CBM-C |
| CBM-29 | 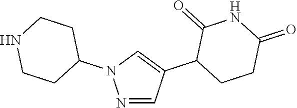 [SMILES: O=C1CCC(c2cnn(C3CCNCC3)c2)C(=O)N1] | General Procedure: CBM-A |
| CBM-30 | 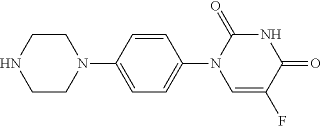 [SMILES: O=c1[nH]c(=O)n(-c2ccc(N3CCNCC3)cc2)cc1F] | Procedure provided below |
| CBM-31 | 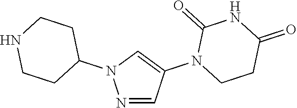 [SMILES: O=C1CCN(c2cnn(C3CCNCC3)c2)C(=O)N1] | Procedure provided below |

### Example S1. General Procedure for CBM-A

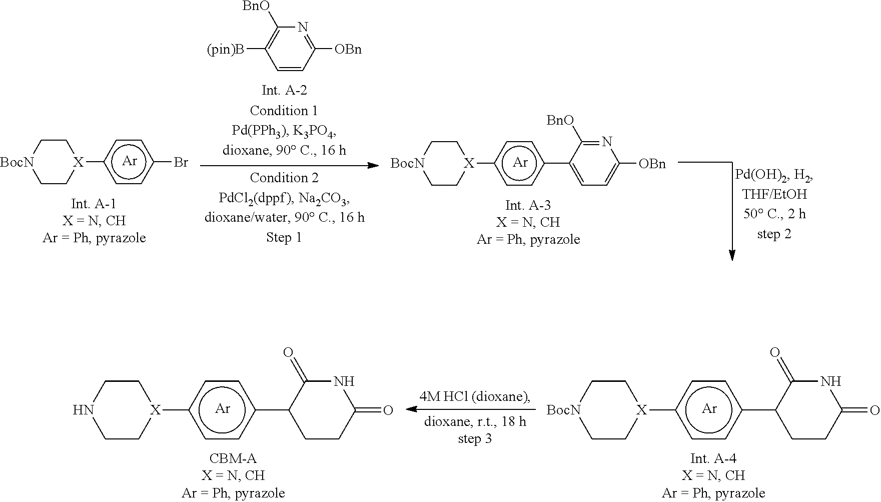

### Synthesis of CBM-A: Step 1

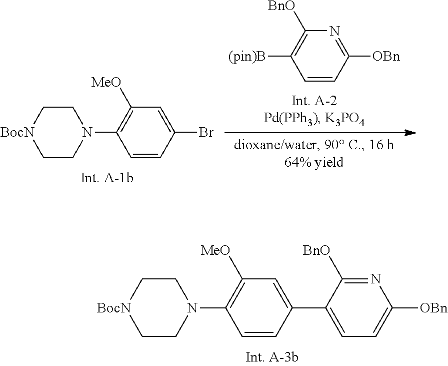

To a sealed tube was added tert-butyl 4-(4-bromo-2-methoxy-phenyl)piperazine-1-carboxylate Int. A-1b (250 mg, 0.67 mmol, 1 eq.), K₃PO₄ (324.85 mg, 1.53 mmol, 2.3 eq.), 2,6-dibenzyloxy-3-(4,4,5,5-tetramethyl-1,3,2-dioxaborolan-2-yl)pyridine Int. A-2 (337.2 mg, 0.81 mmol, 1.2 eq.) and Pd(PPh₃)₄ (77.81 mg, 0.07 mmol, 0.1 eq.). The tube was then flushed with nitrogen for 5 minutes. A mixture of 1,4-dioxane (2.5857 mL, 0.2 M) and water (0.6464 mL, 0.2 M) (degassed by sparging with nitrogen for 10 minutes) was added, and the resulting mixture was sparged with nitrogen for an additional 5 minutes. The vial was sealed and heated overnight at 90° C. for 16 h. LCMS showed complete conversion to Int. A-3b. The crude mixture was passed over celite, washed with DCM and concentrated. The crude residue was purified by silica gel chromatography (0% to 25% EtOAc in heptane). The fractions were combined and concentrated to afford tert-butyl 4-(4-(2,6-bis(benzyloxy)pyridin-3-yl)-2-methoxyphenyl)piperazine-1-carboxylate Int. A-3b (252 mg, 64% yield) as a yellow solid.

LCMS: [M+H]⁺=582.3.

¹H NMR (400 MHz, CHLOROFORM-d) δ ppm 1.50 (s, 9H), 3.03 (t, J=4.8 Hz, 4H), 3.62 (t, J=4.8 Hz, 4H), 3.73 (s, 3H), 5.41 (d, J=7.1 Hz, 4H), 6.48 (d, J=8.1 Hz, 1H), 6.92 (d, J=8.3 Hz, 1H), 7.08 (dd, J=8.1, 1.7 Hz, 1H), 7.18 (d, J=1.7 Hz, 1H), 7.29-7.48 (m, 10H), 7.64 (d, J=8.1 Hz, 1H).

TABLE 4
List of intermediates used in CBM-A: Step 1
Starting Material

| Int. | Structure | Note |
|---|---|---|
| Int. A-1a | 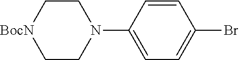 [SMILES: CC(C)(C)OC(=O)N1CCN(c2ccc(Br)cc2)CC1] | Commercially available. |
| Int. A-1b | 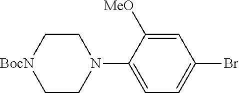 [SMILES: COc1cc(Br)ccc1N1CCN(C(=O)OC(C)(C)C)CC1] | Previously reported: J. Med. Chem. 2014, 57, 1557-1572 |
| Int. A-1c | 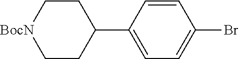 [SMILES: CC(C)(C)OC(=O)N1CCC(c2ccc(Br)cc2)CC1] | Commercially available. |
| Int. A-1d | 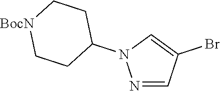 [SMILES: CC(C)(C)OC(=O)N1CCC(n2cc(Br)cn2)CC1] | Commercially available. |
| Int. A-2 | 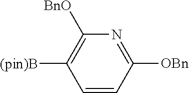 | Synthesis provided below |

TABLE 5
List of Int. A-3 compounds generated by General Procedure CBM-A: Step 1

| Starting | | | | |
|---|---|---|---|---|
| Material | Product | | | |
| Int. A-1 | \|Int. A-3 | Int. A-3 Structure | Characterization | Notes |
| Int. A-1a | Int. A-3a | 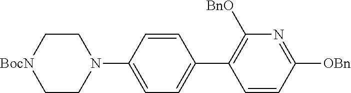 [SMILES: CC(C)(C)OC(=O)N1CCN(c2ccc(-c3ccc(OCc4ccccc4)nc3OCc3ccccc3)cc2)CC1] | quant, green solid ¹H NMR (400 MHz, CDCl₃) δ 7.59- 7.61 (m, 1H), 7.50-7.53 (m, 2H), 7.34-7.43 (m, 10H), 6.96 (d, J = 8.8 Hz, 2H), 6.70 (d, J = 8.0 Hz, 1H), 5.44 (s, 2H), 5.37 (s, 2H), 3.61 (t, J = 5.2 Hz, 4H), 3.18 (t, J = 5.2 Hz, 4H), 1.51 (s, 9H). | Condition 1 |
| Int. A-1b | Int. A-3b | 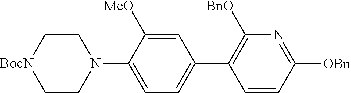 [SMILES: COc1cc(-c2ccc(OCc3ccccc3)nc2OCc2ccccc2)ccc1N1CCN(C(=O)OC(C)(C)C)CC1] | 64% yield, yellow solid [M + H]⁺ = 582.3 ¹H NMR (400 MHz, CHLOROFORM-d) δ ppm 1.50 (s, 9 H), 3.03 (t, J = 4.8 Hz, 4 H), 3.62 (t, J = 4.8 Hz, 4 H), 3.73 (s, 3 H), 5.41 (d, J = 7.1 Hz, 4 H), 6.48 (d, J = 8.1 Hz, 1 H), 6.92 (d, J = 8.3 Hz, 1 H), 7.08 (dd, J = 8.1, 1.7 Hz, 1 H), 7.18 (d, J = 1.7 Hz, 1 H), 7.29-7.48 (m, 10 H), 7.64 (d, J = 8.1 Hz, 1 H). | Condition 1 |
| Int. A-1c | Int. A-3c | 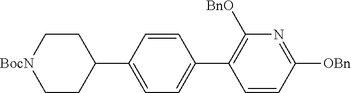 [SMILES: CC(C)(C)OC(=O)N1CCC(c2ccc(-c3ccc(OCc4ccccc4)nc3OCc3ccccc3)cc2)CC1] | [M + H]⁺ = 551. | Condition 1 |
| Int. A-1d | Int. A-3d | 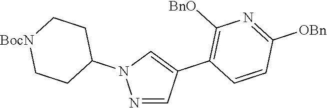 [SMILES: CC(C)(C)OC(=O)N1CCC(n2cc(-c3ccc(OCc4ccccc4)nc3OCc3ccccc3)cn2)CC1] | 47% yield, yellow oil [M + H]⁺ = 541.3. ¹H NMR (400 MHz, DMSO-d6) δ ppm 1.40 (s, 9 H), 1.74 (ddd, J = 24.2, 12.1, 4.6 Hz, 3 H), 1.94-2.04 (m, 3 H), 2.91 (br s, 2 H), 4.28-4.40 (m, 1 H), 5.34 (s, 2 H), 5.46 (s, 2 H), 6.49 (d, J = 8.1 Hz, 1 H), 7.29-7.50 (m, 10 H), 7.87 (s, 1 H), 7.96 (d, J = 8.1 Hz, 1 H), 8.13 (s, 1 H). | Condition 2 |

### Synthesis of 2,6-bis(benzyloxy)-3-(4,4,5,5-tetramethyl-1,3,2-dioxaborolan-2-yl)pyridine (Int A-2)

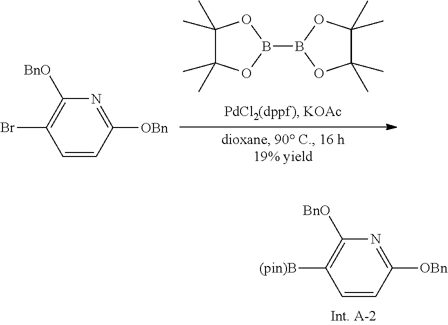

In a sealed tube, 2,6-dibenzyloxy-3-bromo-pyridine (1.0 g, 2.701 mmol, 1.0 eq.), bis(pinacolato)diboron (1.0 g, 4.051 mmol, 1.5 eq.), KOAc (795.2 mg, 8.103 mmol, 3.0 eq.) and Pd(dppf)Cl₂·DCM (220.57 mg, 0.270 mmol, 0.1 eq.) were solubilized in 1,4-dioxane (4.0 mL, 0.6 M) and nitrogen was bubbled for 10 minutes. The tube was sealed and heated at 90° C. overnight. The reaction mixture was cooled down to room temperature. The reaction was filtered over celite, washed with 2-MeTHF and the filtrate was evaporated. The residue was purified by silica gel chromatography (0% to 10% EtOAc in heptane) to afford 900 mg of Int. A-2 contaminated with bis(pinacolato)diboron. A second purification was done by silica gel chromatography (0% to 4.5% EtOAc) to afford 2,6-dibenzyloxy-3-(4,4,5,5-tetramethyl-1,3,2-dioxaborolan-2-yl)pyridine Int. A-2 (210 mg, 19% yield) as a white solid.

LCMS: [M-pin+H]⁺=336.2, [M+H]⁺=418.2.

¹H NMR (400 MHz, DMSO-d₆) δ ppm 1.28 (s, 12H), 5.38 (d, J=5.9 Hz, 4H), 6.42 (d, J=7.8 Hz, 1H), 7.24-7.40 (m, 6H), 7.40-7.44 (m, 2H), 7.53 (d, J=7.1 Hz, 2H), 7.84 (d, J=7.8 Hz, 1H).

### Synthesis of CBM-A: Step 2

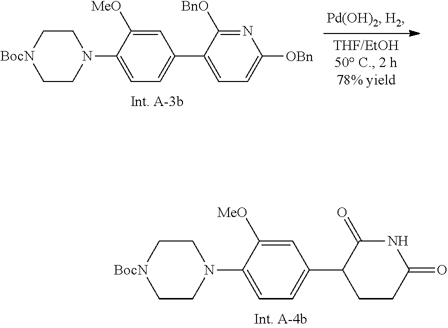 [SMILES: COc1cc(-c2ccc(OCc3ccccc3)nc2OCc2ccccc2)ccc1N1CCN(C(=O)OC(C)(C)C)CC1 ; COc1cc(C2CCC(=O)NC2=O)ccc1N1CCN(C(=O)OC(C)(C)C)CC1]

A solution of tert-butyl 4-[4-(2,6-dibenzyloxy-3-pyridyl)-2-methoxy-phenyl]piperazine-1-carboxylate Int. A-3b (155 mg, 0.27 mmol, 1 eq.) in THE (1.5 mL, 0.09 M) and ethanol (1.5 mL, 0.09 M) was degassed for 15 minutes, then Pd(OH)₂ (37.42 mg, 0.05 mmol, 0.2 eq.) was added and sparging was resumed for 5 minutes. Hydrogen was then bubbled in the reaction mixture for 5 minutes and the mixture was stirred in a hot water bath at 50° C. under hydrogen atmosphere. After 2 h., LCMS showed full conversion into compound Int. A-4b. The crude mixture was passed over celite, washed with DCM and concentrated. The crude product was purified by reverse phase column chromatography (5% to 50% MeCN in water w/0.1% formic acid). The fractions were combined and concentrated to give tert-butyl 4-(4-(2,6-dioxopiperidin-3-yl)-2-methoxyphenyl)piperazine-1-carboxylate Int. A-4b (84 mg, 78% yield) as a yellow solid.

LCMS: [M+H]⁺=404.4.

¹H NMR (400 MHz, DMSO-d₆) δ ppm 1.50 (s, 9H), 3.03 (t, J 4.8 Hz, 4H), 3.62 (t, J=4.8 Hz, 4H), 3.73 (s, 3H), 5.41 (d, J=7.1 Hz, 4H), 6.48 (d, J=8.1 Hz, 1H), 6.92 (d, J=8.3 Hz, 1H), 7.08 (dd, J=8.1, 1.7 Hz, 1H), 7.18 (d, J=1.7 Hz, 1H), 7.29-7.48 (m, 10H), 7.64 d. (J=8.1 Hz, 1H).

TABLE 6
List of Int. A-4 compounds generated by General Procedure CBM-A: Step 2

| Starting | | | | |
|---|---|---|---|---|
| Material | Product | | | |
| Int. A-3 | Int. A-4 | Structure | Characterization | Notes |
| Int. A-3a | Int. A-4a | 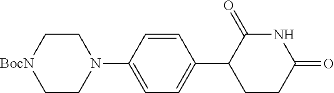 [SMILES: CC(C)(C)OC(=O)N1CCN(c2ccc(C3CCC(=O)NC3=O)cc2)CC1] | 80% yield, white solid ¹H NMR (400 MHz, CDCl₃) δ ppm 10.8 (s, 1H), 7.07 (d, J = 8.8 Hz, 2H), 6.92 (d, J = 8.8 Hz, 2H), 3.74 (dd, J = 11.2 Hz, 6.4 Hz, 1H), 3.44-3.45 (m, 4H), 3.07 (t, J = 6.4 Hz, 4H), 2.61-2.64 (m, 1H), 2.44-2.48 (m, 1H), 2.13 (m, 1H), 2.01- 2.02 (m, 1H), 1.42 (s, 9H). | 10% Pd/C and 1.0 equiv. of AcOH were added to reaction |
| Int. A-3b | Int. A-4b | 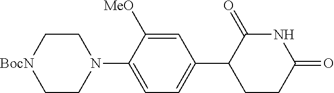 [SMILES: COc1cc(C2CCC(=O)NC2=O)ccc1N1CCN(C(=O)OC(C)(C)C)CC1] | 78% yield, yellow solid [M + H]⁺ = 404.4. ¹H NMR (400 MHz, DMSO- d6) δ ppm 1.50 (s, 9 H), 3.03 (t, J = 4.8 Hz, 4 H), 3.62 (t, J = 4.8 Hz, 4 H), 3.73 (s, 3 H), 5.41 (d, J = 7.1 Hz, 4 H), 6.48 (d, J = 8.1 Hz, 1 H), 6.92 (d, J = 8.3 Hz, 1 H), 7.08 (dd, J = 8.1, 1.7 Hz, 1 H), 7.18 (d, J = 1.7 Hz, 1 H), 7.29-7.48 (m, 10 H), 7.64 (d, J = 8.1 Hz, 1 H). | |
| Int. A-3c | Int. A-4c | 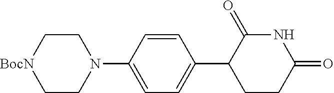 [SMILES: CC(C)(C)OC(=O)N1CCN(c2ccc(C3CCC(=O)NC3=O)cc2)CC1] | [M + H]⁺ = 373. | 10 h reaction time |
| Int. A-3d | Int. A-4d | 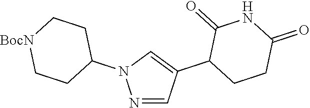 [SMILES: CC(C)(C)OC(=O)N1CCC(n2cc(C3CCC(=O)NC3=O)cn2)CC1] | 75% yield, off-white semi-solid [M + Na]⁺ = 363.2 ¹H NMR (400 MHz, DMSO- d6) δ ppm 1.41 (s, 9 H), 1.72 (ddd, J = 24.5, 12.5, 4.4 Hz, 2 H), 1.90-2.00 (m, 2 H), 2.02- 2.16 (m, 3 H), 2.56-2.65 (m, 1 H), 2.88 (br s, 2 H), 3.73 (dd, J = 10.0, 5.1 Hz, 1 H), 4.02 (br d, J = 12.0 Hz, 2 H), 4.26-4.36 (m, 1 H), 7.37 (s, 1 H), 7.71 (s, 1 H), 10.73 (s, 1 H). | 60 psi of H₂ used, 16 h reaction time, 60° C. |

### Synthesis of General Procedure for CBM-A: Step 3

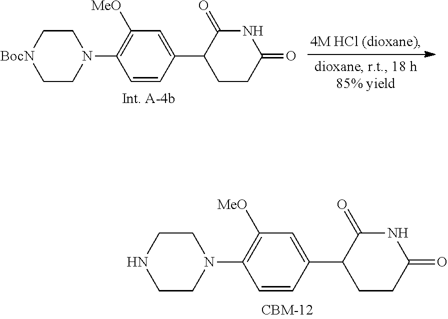 [SMILES: COc1cc(C2CCC(=O)NC2=O)ccc1N1CCN(C(=O)OC(C)(C)C)CC1 ; COc1cc(C2CCC(=O)NC2=O)ccc1N1CCNCC1]

To a solution of tert-butyl 4-[4-(2,6-dioxo-3-piperidyl)-2-methoxy-phenyl]piperazine-1-carboxylate 2nt. A-4b (130 mg, 0.32 mmol, 1 eq.) in DCM (1.4 mL, 0.23 M) was added 4 M HCl in 1,4-dioxane (1.2 mL, 4.82 mmol, 15 eq.). The reaction mixture was stirred at room temperature overnight. LCMS showed full conversion into compound CBM-12. The reaction mixture was concentrated under reduced pressure and the residue was co-evaporated with MeOH (2×) and MTBE (2×) to give 3-(3-methoxy-4-(piperazin-1-yl)phenyl)piperi dine-2,6-dione CBM-12 (103 mg, 85%8 yield) as a tan solid as a bis HCl salt.

LCMS: [M+H]⁺=304.2.

¹H NMR (400 MHz, DMSO-d₆) δ ppm 1.94-2.06 (m, 1H), 2.14-2.29 (m, 1H), 2.43-2.49 (m, 1H), 2.58-2.71 (m, 1H), 3.17 (br s, 4H), 3.18-3.25 (m, 4H), 3.75-3.82 (m, 4H), 6.71-6.77 (m, 1H), 6.84-6.91 (m, 2H), 9.08 (br s, 2H), 10.80 (s, 1H).

TABLE 7
List of CBM-A compounds generated by General Procedure CBM-A: Step 3

| Starting | | | | |
|---|---|---|---|---|
| Material | Product | | | |
| Int. A-4 | CBM | Structure | Characterization | Notes |
| Int. A-4a | CBM-2 | 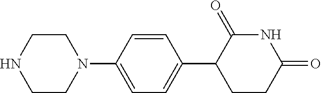 [SMILES: O=C1CCC(c2ccc(N3CCNCC3)cc2)C(=O)N1] | quant. yield, white solid ¹H NMR (400 MHz, DMSO-d6) δ 10.8 (s, 1H), 9.24 (s, 2H), 7.11 (d, J = 8.4 Hz, 2H), 6.96 (d, J = 8.8 Hz, 2H), 3.77 (q, J = 5.2 Hz, 1H), 3.34- 3.36 (m, 4H), 3.20 (s, 4H), 2.61- 2.64 (m, 1H), 2.44-2.48 (m, 1H), 2.14 (m, 1H), 1.90-2.01 (m, 1H). | 3 h reaction time |
| Int. A-4b | CBM-12 | 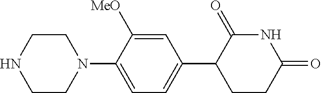 [SMILES: COc1cc(C2CCC(=O)NC2=O)ccc1N1CCNCC1] | 85% yield, tan solid LCMS: [M + H]⁺ = 304.2 ¹H NMR (400 MHz, DMSO-d6) δ ppm 1.94-2.06 (m, 1 H), 2.14-2.29 (m, 1 H), 2.43-2.49 (m, 1 H), 2.58- 2.71 (m, 1 H), 3.17 (br s, 4 H), 3.18- 3.25 (m, 4 H), 3.75-3.82 (m, 4 H), 6.71-6.77 (m, 1 H), 6.84-6.91 (m, 2 H), 9.08 (br s, 2 H), 10.80 (s, 1 H). | |
| Int. A-4d | CBM-24 | 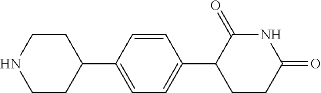 [SMILES: O=C1CCC(c2ccc(C3CCNCC3)cc2)C(=O)N1] | [M + H]⁺ 273 | 2 h reaction time |
| Int. A-4c | CBM-29 | 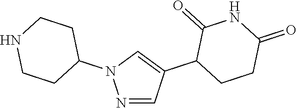 [SMILES: O=C1CCC(c2cnn(C3CCNCC3)c2)C(=O)N1] | quant. yield, white solid [M + H]⁺ = 263.2. ¹H NMR (400 MHz, DMSO-d6) δ ppm 1.99-2.24 (m, 7 H), 2.56-2.67 (m, 1 H), 2.97-3.12 (m, 2 H), 3.32- 3.42 (m, 2 H), 3.76 (dd, J = 10.0, 5.1 Hz, 1 H), 4.39-4.50 (m, 1 H), 7.42 (s, 1 H), 7.69 (s, 1 H), 8.72-8.91 (m, 1 H), 9.04-9.12 (m, 1 H), 10.76 (s, 1 H). | |

### Example S2. General Procedure for CBM-B

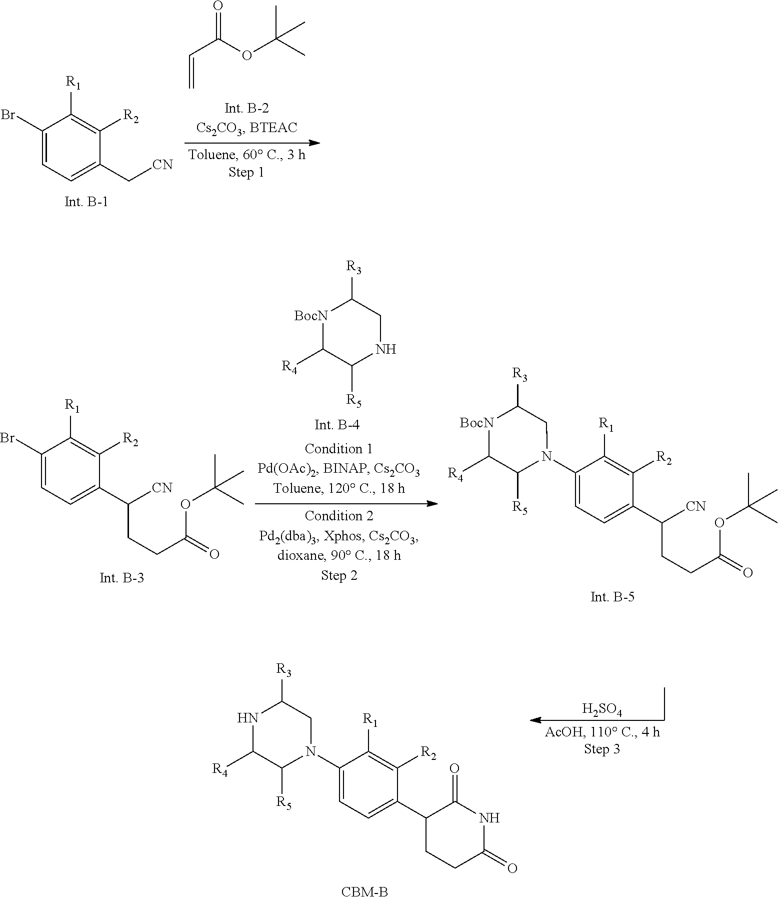

### Synthesis of CBM-B: Step 1

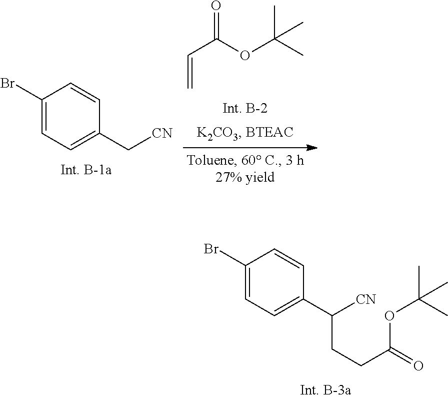

To a solution of 2-(4-bromophenyl)acetonitrile Int. B-1a (25.0 g, 127.5 mmol, 1.0 eq.) in toluene (255 mL) was added tert-butyl prop-2-enoate Int. B-2 (18.7 mL, 127.5 mmol, 1.0 eq.), N-benzyl-N,N-diethylethanaminium chloride (BTEAC) (2.9 g, 12.7 mmol, 0.1 eq.), and potassium carbonate (17.6 g, 127.5 mmol, 1.0 eq.). The mixture was stirred at 65° C. under nitrogen for 3 hours, then cooled to room temperature and filtered through a glass fritted funnel. The mixture was concentrated, affording a crude mixture which was purified by reverse phase flash chromatography (5% to 100% MeCN in water w/0.1% formic acid), resulting in 12.8 g (27% yield) of tert-butyl 4-(4-bromophenyl)-4-cyano-butanoate Int. B-3a as a light-yellow oil.

LCMS: [M+H]+=324.2.

¹H NMR (400 MHz, DMSO-d₆) δ ppm 1.37 (s, 3H), 1.97-2.15 (m, 2H), 2.28 (t, J=7.5 Hz, 2H), 4.25 (t, J=7.4 Hz, 1H), 7.35 (d, J=8.3 Hz, 2H), 7.62 (d, J=8.3 Hz, 2H).

TABLE 8
List of Int. B-1 compounds used in CBM-B: Step 1
Starting Material

| Int. B-1 | Int. B-1 Structure |
|---|---|
| Int. B-1a | 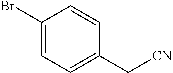 |
| Int. B-1b | 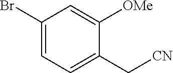 [SMILES: COc1cc(Br)ccc1CC#N] |
| Int. B-1c | 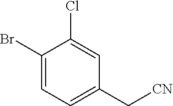 [SMILES: N#CCc1ccc(Br)c(Cl)c1] |
| Int. B-1d | 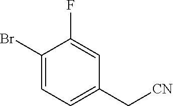 [SMILES: N#CCc1ccc(Br)c(F)c1] |
| Int. B-1e | 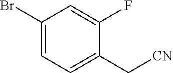 [SMILES: N#CCc1ccc(Br)cc1F] |
| Int. B-1f | 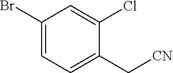 [SMILES: N#CCc1ccc(Br)cc1Cl] |

TABLE 9
List of Int. B-3 compounds generated by General Procedure CBM-B: Step 1

| Starting | Product | | | |
|---|---|---|---|---|
| Material | | Int. B-3 | | |
| Int. B-1 | Int. B-3 | Structure | Characterization | Notes |
| Int. B-1a | Int. B-3a | 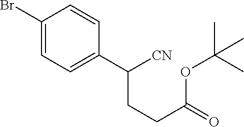 [SMILES: CC(C)(C)OC(=O)CCC(C#N)c1ccc(Br)cc1] | 27% yield, light-yellow oil [M + H] = 324.2 ¹H NMR (400 MHz, DMSO-d6) δ ppm 1.37 (s, 3 H), 1.97-2.15 (m, 2 H), 2.28 (t, J = 7.5 Hz, 2 H), 4.25 (t, J = 7.4 Hz, 1 H), 7.35 (d, J = 8.3 Hz, 2 H), 7.62 (d, J = 8.3 Hz, 2 H). | K₂CO₃ was used instead of Cs₂CO₃ |
| Int. B-1b | Int. B-3b | 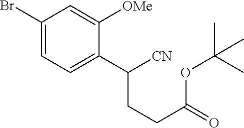 [SMILES: COc1cc(Br)ccc1C(C#N)CCC(=O)OC(C)(C)C] | 43% yield, colorless oil [M + H]⁺ = 354.2 ¹H NMR (400 MHz, DMSO-d6) δ ppm 1.38 (s, 9 H), 1.94-2.15 (m, 2 H), 2.20-2.34 (m, 2 H), 3.86 (s, 3 H), 4.29 (t, J = 7.5 Hz, 1 H), 7.19- 7.23 (m, 1 H), 7.26-7.30 (m, 2 H). | |
| Int. B-1c | Int. B-3c | 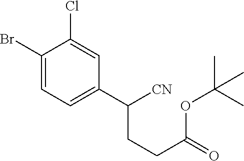 [SMILES: CC(C)(C)OC(=O)CCC(C#N)c1ccc(Br)c(Cl)c1] | 82% yield, colorless oil [M + H]⁺ = 358.0, 360.0 ¹H NMR (400 MHz, CHLOROFORM-d) δ ppm 1.45 (s, 9 H), 2.08-2.25 (m, 2 H), 2.36-2.53 (m, 2 H), 4.39 (dd, J = 8.6, 6.1 Hz, 1 H), 7.40-7.44 (m, 1 H), 7.46-7.51 (m, 1 H), 7.60 (d, J = 1.7 Hz, 1 H). | K₂CO₃ was used instead of Cs₂CO₃ |
| Int. B-1d | Int. B-3d | 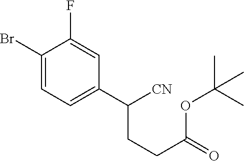 [SMILES: CC(C)(C)OC(=O)CCC(C#N)c1ccc(Br)c(F)c1] | 26% yield, light yellow oil [M + H]⁺ = 342.2 ¹H NMR (400 MHz, CHLOROFORM-d) δ ppm 1.46 (s, 9 H), 2.09-2.21 (m, 2 H), 2.32-2.54 (m, 2 H), 3.99 (t, J = 7.6 Hz, 1 H), 7.03-7.10 (m, 1 H), 7.15 (dd, J = 8.9, 2.1 Hz, 1 H), 7.59 (dd, J = 8.3, 7.1 Hz, 1 H). | |
| Int. B-le | Int. B-3e | 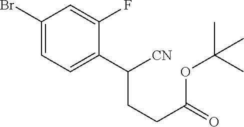 [SMILES: CC(C)(C)OC(=O)CCC(C#N)c1ccc(Br)cc1F] | 38% yield, yellow oil [M + H]⁺ = 342.2 ¹H NMR (400 MHz, Chloroform-d) 8 ppm 1.45 (s, 9 H), 2.10-2.26 (m, 2 H), 2.33-2.50 (m, 2 H), 4.23 (t, J = 7.5 Hz, 1 H), 7.29-7.40 (m, 3 H). ¹⁹F NMR (377 MHz, Chloroform-d) 8 ppm-115.02 (dd, J = 9.5, 6.8 Hz, 1 F). | |
| Int. B-1f | Int. B-1f | 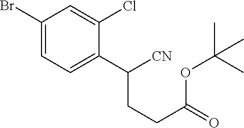 [SMILES: CC(C)(C)OC(=O)CCC(C#N)c1ccc(Br)cc1Cl] | 66% yield, yellow oil [M + H]⁺ = 358.2 ¹H NMR (400 MHz, Chloroform-d) 8 ppm 1.45 (s, 9 H), 2.08-2.25 (m, 2 H), 2.37-2.53 (m, 2 H), 4.39 (dd, J = 8.6, 6.1 Hz, 1 H), 7.40-7.45 (m, 1 H), 7.47-7.52 (m, 1 H), 7.60 (s, 1 H). | |

### Synthesis of CBM-B: Step 2

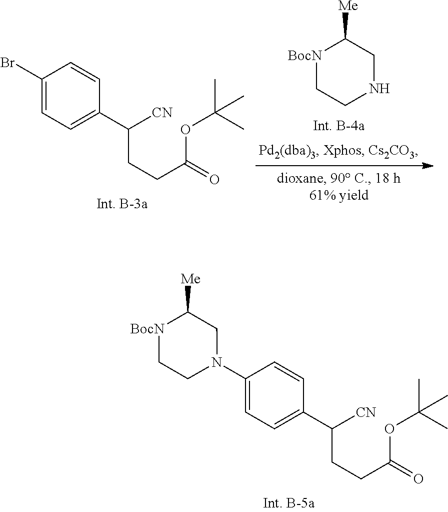 [SMILES: CC(C)(C)OC(=O)CCC(C#N)c1ccc(Br)cc1 ; C[C@H]1CNCCN1C(=O)OC(C)(C)C ; C[C@H]1CN(c2ccc(C(C#N)CCC(=O)OC(C)(C)C)cc2)CCN1C(=O)OC(C)(C)C]

To a solution of tert-butyl (2S′)-2-methylpiperazine-1-carboxylate Int. B-4a (340 mg, 1.7 mmol, 1.1 equiv.) in 1,4-dioxane (3.00 mL, 0.5 M) were added Cs₂CO₃ (1.01 g, 3.08 mmol, 2.0 equiv.), tert-butyl 4-(4-bromophenyl)-4-cyano-butanoate Int. B-3a (500. mg, 1.54 mmol, 1 equiv.), Xphos (147 mg, 0.31 mmol, 0.2 equiv.), Pd₂(dba)₃ (141 mg, 0.15 mmol, 0.1 equiv.). The reaction mixture was degassed with N₂ for 15 min and stirred at 90° C. After 18 h the LCMS showed full conversion into Int. B-5a. The reaction mixture was filtered through a pad of celite and washed with EtOAc. The filtrate was concentrated under vacuum and the residue was purified by silica gel chromatography (0% to 80% EtOAc in heptane). The fractions were combined and concentrated to give tert-butyl (2S)-4-(4-(4-(tert-butoxy)-1-cyano-4-oxobutyl)phenyl)-2-methylpiperazine-1-carboxylate Int. B-5a (452 mg, 61% yield) as a brown oil.

LCMS: 93.4% purity at 215 nm, [M+H]⁺=444.4.

¹H NMR (400 MHz, DMSO-d₆) δ ppm 1.18 (d, J=6.6 Hz, 3H), 1.38 (s, 9H), 1.42 (s, 9H), 1.92-2.11 (m, 2H), 2.26 (t, J=8.0 Hz, 2H), 2.58-2.68 (m, 1H), 2.83 (dd, J=12.2, 3.7 Hz, 1H), 3.09-3.20 (m, 1H), 3.50 (br d, J=12.0 Hz, 1H), 3.59 (br d, J=11.7 Hz, 1H), 3.79 (br d, J=13.0 Hz, 1H), 4.08 (t, J=7.3 Hz, 1H), 4.15-4.25 (m, 1H), 6.94 (d, J=8.6 Hz, 2H), 7.20 (d, J=8.8 Hz, 2H).

TABLE 10
List of Int. B-4 compounds used in CBM-B: Step 2
Starting Materials

| Int. B-4 | Int. B-4 Structure |
|---|---|
| Int. B-4a | 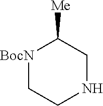 [SMILES: C[C@H]1CNCCN1C(=O)OC(C)(C)C] |
| Int. B-4b | 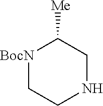 [SMILES: C[C@@H]1CNCCN1C(=O)OC(C)(C)C] |
| Int. B-4c | 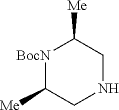 [SMILES: C[C@@H]1CNC[C@H](C)N1C(=O)OC(C)(C)C] |
| Int. B-4d | 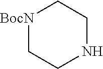 [SMILES: CC(C)(C)OC(=O)N1CCNCC1] |
| Int. B-4e | 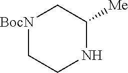 [SMILES: C[C@H]1CN(C(=O)OC(C)(C)C)CCN1] |
| Int. B-4f | 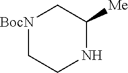 [SMILES: C[C@@H]1CN(C(=O)OC(C)(C)C)CCN1] |
| Int. B-4g | 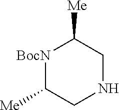 [SMILES: C[C@H]1CNC[C@H](C)N1C(=O)OC(C)(C)C] |
| Int. B-4h | 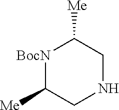 [SMILES: C[C@@H]1CNC[C@@H](C)N1C(=O)OC(C)(C)C] |

TABLE 11
List of Int. B-5 compounds generated by General Procedure CBM-B: Step 2

| Starting | | | | | |
|---|---|---|---|---|---|
| Materials | Product | | | | |
| Int. | Int. | Int. | | | |
| B-3 | B-4 | B-5 | Int. B-5 Structure | Characterization | Notes |
| Int. B-3a | Int. B-4a | Int B-5a | 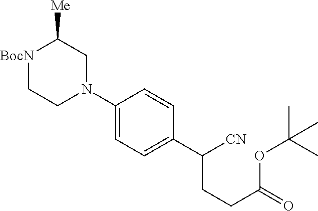 [SMILES: C[C@H]1CN(c2ccc(C(C#N)CCC(=O)OC(C)(C)C)cc2)CCN1C(=O)OC(C)(C)C] | 61% yield, brown oil [M + H]⁺ = 444.4 ¹H NMR (400 MHz, DMSO-d6) δ ppm 1.18 (d, J = 6.6 Hz, 3 H), 1.38 (s, 9 H), 1.42 (s, 9 H), 1.92-2.11 (m, 2 H), 2.26 (t, J = 8.0 Hz, 2 H), 2.58- 2.68 (m, 1 H), 2.83 (dd, J = 12.2, 3.7 Hz, 1 H), 3.09- 3.20 (m, 1 H), 3.50 (br d, J = 12.0 Hz, 1 H), 3.59 (br d, J = 11.7 Hz, 1 H), 3.79 (br d, J = 13.0 Hz, 1 H), 4.08 (t, J = 7.3 Hz, 1 H), 4.15-4.25 (m, 1 H), 6.94 (d, J = 8.6 Hz, 2 H), 7.20 (d, J = 8.8 Hz, 2 H). | Condition 2 |
| Int. B-3a | Int. B-4b | Int. B-5b | 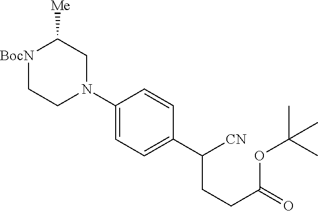 [SMILES: C[C@@H]1CN(c2ccc(C(C#N)CCC(=O)OC(C)(C)C)cc2)CCN1C(=O)OC(C)(C)C] | 89% yield, purple oil [M + H]⁺ = 444.4 ¹H NMR (400 MHz, CHLOROFORM-d) δ ppm 1.29-1.32 (m, 3 H), 1.43-1.46 (m, 9 H), 1.48- 1.51 (m, 9 H), 2.09- 2.18 (m, 2 H), 2.30-2.47 (m, 2 H), 2.77 (td, J = 11.7, 3.4 Hz, 1 H), 2.97 (dd, J = 12.1, 3.8 Hz, 1 H), 3.26 (td, J = 12.6, 3.4 Hz, 1 H), 3.38 (br d, J = 12.0 Hz, 1 H), 3.52 (br d, J = 12.0 Hz, 1 H), 3.87 (t, J = 7.5 Hz, 1 H), 3.95 (br d, J = 13.2 Hz, 1 H), 4.35 (s, 1 H), 6.88 (d, J = 8.6 Hz, 2 H), 7.22 (d, J = 8.8 Hz, 2 H). | Condition 2 |
| Int. B-3a | Int. B-4c | Int B-5c | 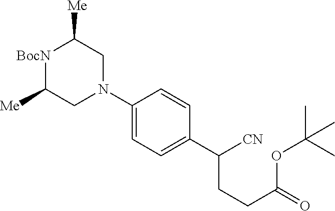 [SMILES: C[C@@H]1CN(c2ccc(C(C#N)CCC(=O)OC(C)(C)C)cc2)C[C@H](C)N1C(=O)OC(C)(C)C] | 66% yield, orange oil [M + H]⁺ = 458.4 ¹H NMR (400 MHz, DMSO-d6) δ ppm 1.23 (d, J = 6.8 Hz, 7 H), 1.38 (s, 9 H), 1.39-1.40 (m, 1 H), 1.43 (s, 9 H), 1.96- 2.10 (m, 2 H), 2.23-2.29 (m, 2 H), 2.79 (br dd, J = 12.2, 4.2 Hz, 2 H), 3.51 (br d, J = 12.2 Hz, 2 H), 4.05-4.13 (m, 3 H), 6.98 (d, J = 8.6 Hz, 2 H), 7.21 (d, J = 8.6 Hz, 2 H) | Condition 2 |
| Int. B-3b | Int. B-4d | Int. B-5d | 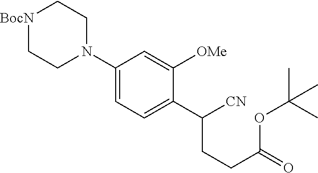 [SMILES: COc1cc(N2CCN(C(=O)OC(C)(C)C)CC2)ccc1C(C#N)CCC(=O)OC(C)(C)C] | 43% yield, red oil [M + H]⁺ = 460.4 ¹H NMR (400 MHz, Chloroform-d) δ ppm 1.44 (s, 9 H), 1.50 (s, 9 H), 2.04-2.23 (m, 2 H), 2.30-2.41 (m, 2 H), 3.16 (m, 4 H), 3.57-3.62 (m, 4 H), 3.84 (s, 3 H), 4.19 (t, J = 7.2 Hz, 1 H), 6.44 (s, 1 H), 6.51 (d, J = 8.3 Hz, 1 H), 7.24 (d, J = 8.3 Hz, 1 H). | Condition 1 |
| Int. B-3c | Int. B-4d | Int. B-5e | 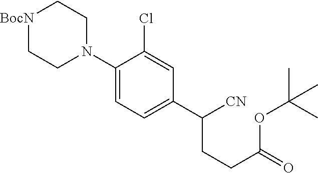 [SMILES: CC(C)(C)OC(=O)CCC(C#N)c1ccc(N2CCN(C(=O)OC(C)(C)C)CC2)c(Cl)c1] | 58% yield, red oil [M + H]⁺ = 464.4 ¹H NMR (400 MHz, DMSO-d6) δ ppm 1.37- 1.39 (m, 9 H), 1.41-1.43 (m, 9 H), 1.98-2.16 (m, 2 H), 2.22-2.35 (m, 3 H), 2.89-2.96 (m, 4 H), 3.15-3.21 (m, 1 H), 3.40- 3.44 (m, 1 H), 3.45- 3.50 (m, 4 H), 4.20 (t, J = 7.6 Hz, 1 H), 7.20 (d, J = 8.3 Hz, 1 H), 7.30-7.37 (m, 1 H), 7.43-7.46 (m, 1 H). | Condition 2 |
| Int. B-3a | Int. B-4e | Int. B-5f | 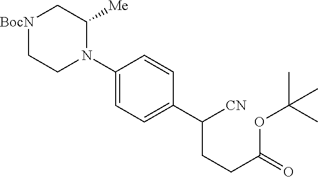 [SMILES: C[C@H]1CN(C(=O)OC(C)(C)C)CCN1c1ccc(C(C#N)CCC(=O)OC(C)(C)C)cc1] | 72% yield, red oil [M + H]⁺ = 444.4 ¹H NMR (400 MHz, CHLOROFORM-d) δ ppm 0.99-1.05 (m, 3 H), 1.45 (s, 9 H), 1.49 (s, 9 H), 2.08-2.21 (m, 2 H), 2.29-2.47 (m, 2 H), 3.03- 3.26 (m, 3 H), 3.27- 3.37 (m, 1 H), 3.76-3.92 (m, 3 H), 3.93-4.19 (m, 1 H), 6.87 (d, J = 8.6 Hz, 2 H), 7.21 (d, J = 8.6 Hz, 2 H). | Condition 2 |
| Int. B-3d | Int. B-4d | Int. B-5g | 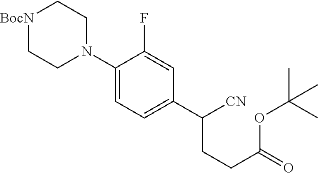 [SMILES: CC(C)(C)OC(=O)CCC(C#N)c1ccc(N2CCN(C(=O)OC(C)(C)C)CC2)c(F)c1] | 35% yield, yellow oil [M + H]⁺ = 448.3 ¹H NMR (400 MHz, CHLOROFORM-d) δ ppm 1.50 (s, 9 H), 2.07- 2.21 (m, 1 H), 2.31-2.49 (m, 1 H), 2.67 (t, J = 6.7 Hz, 1 H), 3.00-3.08 (m, 2 H), 3.13-3.21 (m, 4 H), 3.57-3.66 (m, 4 H), 6.88-6.96 (m, 1 H), 7.00- 7.09 (m, 1 H), 7.67- 7.75 (m, 1 H). ¹⁹F NMR (377 MHz, CHLOROFORM-d) δ ppm −121.32 (dd, J = 13.6, 8.2 Hz, 1 F). | Condition 1 |
| Int. B-3a | Int. B-4f | Int. B-5h | 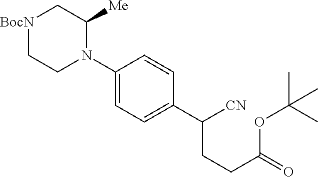 [SMILES: C[C@@H]1CN(C(=O)OC(C)(C)C)CCN1c1ccc(C(C#N)CCC(=O)OC(C)(C)C)cc1] | yield n.d., 41% pure, used in next step [M + H]⁺ = 444.4 | Condition 2 |
| Int. B-3e | Int. B-4d | Int. B-5i | 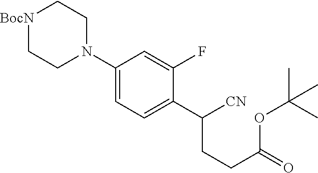 [SMILES: CC(C)(C)OC(=O)CCC(C#N)c1ccc(N2CCN(C(=O)OC(C)(C)C)CC2)cc1F] | 80% yield, yellow semi- solid [M − Boc + H]⁺ = 348.4 ¹H NMR (400 MHz, Chloroform-d) δ ppm 1.45 (s, 9 H), 1.49 (s, 9 H), 2.06-2.25 (m, 2 H), 2.30-2.49 (m, 2 H), 3.12- 3.22 (m, 4 H), 3.55- 3.62 (m, 4 H), 4.13 (t, J = 7.3 Hz, 1 H), 6.59 (d, J = 13.4 Hz, 1 H), 6.70 (d, J = 8.7 Hz, 1 H), 7.28 (t, J = 8.7 Hz, 1 H). ¹⁹F NMR (377 MHz, Chloroform-d) δ ppm- 116.69 (dd, J = 12.9, 8.9 Hz, 1 F). | Condition 1 |
| Int. B-3f | Int. B-4d | Int B-5j | 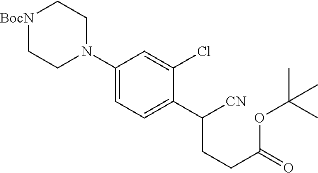 [SMILES: CC(C)(C)OC(=O)CCC(C#N)c1ccc(N2CCN(C(=O)OC(C)(C)C)CC2)cc1Cl] | 73%, brown semi-solid. [M − 2tBu + 3H]⁺ = 352.4 ¹H NMR (400 MHz, Chloroform-d) δ ppm 1.45 (s, 9 H), 1.49 (s, 9 H), 2.10-2.22 (m, 2 H), 2.35-2.49 (m, 2 H), 3.11- 3.23 (m, 4 H), 3.55- 3.61 (m, 4 H), 4.32 (t, J = 7.3 Hz, 1 H), 6.84 (d, J = 8.6 Hz, 1 H), 6.90 (s, 1 H), 7.38 (d, J = 8.6 Hz, 1 H). | Condition 1 |
| Int. B-3a | Int. B-4g | Int. B-5k | 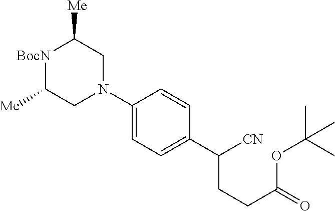 [SMILES: C[C@H]1CN(c2ccc(C(C#N)CCC(=O)OC(C)(C)C)cc2)C[C@H](C)N1C(=O)OC(C)(C)C] | 66% yield, orange oil [M + Na + H]⁺ = 480.4 ¹H NMR (400 MHz, DMSO-d6) δ ppm 1.14- 1.17 (m, 6 H), 1.38 (s, 9 H), 1.43 (s, 9 H), 1.93- 2.11 (m, 4 H), 2.20-2.31 (m, 2 H), 3.33-3.36 (m, 1 H), 3.62 (dd, J = 12.3, 3.8 Hz, 2 H), 4.06-4.13 (m, 2 H), 6.68 (d, J = 8.6 Hz, 2 H), 7.17 (d, J = 8.6 Hz, 2 H). | Condition 2 |
| Int. B-3a | Int. B-4h | Int. B-5l | 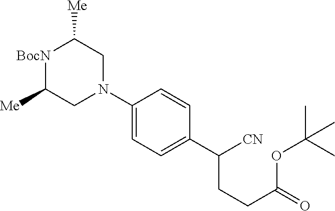 [SMILES: C[C@@H]1CN(c2ccc(C(C#N)CCC(=O)OC(C)(C)C)cc2)C[C@@H](C)N1C(=O)OC(C)(C)C] | 43% yield, yellow oil [M + Na]⁺ = 480.4 ¹H NMR (400 MHz, DMSO-d6) δ ppm 1.14 (s, 3 H), 1.16 (s, 3 H), 1.38 (s, 9 H), 1.43 (s, 9 H), 1.92-2.12 (m, 2 H), 2.22-2.30 (m, 2 H), 3.35 (s, 1 H), 3.62 (dd, J = 12.3, 3.5 Hz, 2 H), 4.00- 4.15 (m, 4 H), 6.68 (d, J = 8.8 Hz, 2 H), 7.15-7.20 (m, 2 H). | Condition 1 |

### Synthesis of CBM-B: Step 3

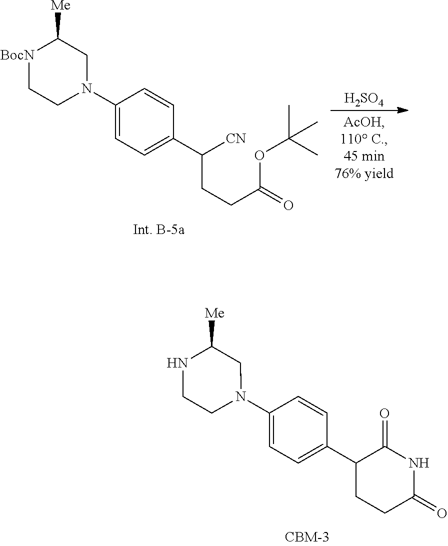 [SMILES: C[C@H]1CN(c2ccc(C(C#N)CCC(=O)OC(C)(C)C)cc2)CCN1C(=O)OC(C)(C)C ; "H2SO4" ; C[C@H]1CN(c2ccc(C3CCC(=O)NC3=O)cc2)CCN1]

To a solution of tert-butyl (2S)-4-[4-(4-tert-butoxy-1-cyano-4-oxo-butyl)phenyl]-2-methyl-piperazine-1-carboxylate Int. B-5a (400 mg, 0.90 mmol, 1.0 equiv.) in acetic acid (4.50 mL, 0.2 M) was added concentrated sulfuric acid (0.14 mL, 2.71 mmol, 3.0 equiv.). The reaction mixture was stirred at 110° C. After 45 min LCMS showed full conversion into CBM-3. The solvent was evaporated, and the residue was then purified by reverse phase flash chromatography (5% to 65% MeCN in water). The fractions were combined and concentrated to give 3-(4-((S)-3-methylpiperazin-1-yl)phenyl)piperidine-2,6-dione CBM-3 (207 mg, 76% yield) as a brown solid as bis sulfuric acid salt.

LCMS: [M+H]⁺=288.2.

¹H NMR (400 MHz, DMSO-d₆) δ ppm 1.23 (br d, J=6.1 Hz, 3H), 1.81 (s, 1H), 1.94-2.05 (m, 1H), 2.06-2.21 (m, 1H), 2.57-2.71 (m, 2H), 2.79-2.91 (m, 1H), 3.03-3.13 (m, 1H), 3.15 (s, 1H), 3.26-3.35 (m, 3H), 3.63-3.79 (m, 3H), 4.75 (br d, J=15.2 Hz, 1H), 6.95 (br d, J=8.3 Hz, 2H), 7.09 (br d, J=8.3 Hz, 2H), 10.78 (s, 1H).

TABLE 12
List of CBM-B compounds generated by General Procedure CBM-B: Step 3

| Starting | | | |
|---|---|---|---|
| Material | Product | | |
| Int. B-5 | CBM | CBM Structure | Characterization |
| Int. B-5a | CBM-3 | 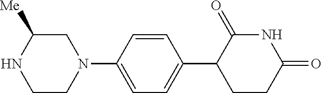 [SMILES: C[C@H]1CN(c2ccc(C3CCC(=O)NC3=O)cc2)CCN1] | 76% yield, brown solid [M + H]⁺ = 288.2 ¹H NMR (400 MHz, DMSO-d6) δ ppm 1.23 (br d, J = 6.1 Hz, 3 H), 1.81 (s, 1 H), 1.94- 2.05 (m, 1 H), 2.06-2.21 (m, 1 H), 2.57- 2.71 (m, 2 H), 2.79-2.91 (m, 1 H), 3.03- 3.13 (m, 1 H), 3.15 (s, 1 H), 3.26-3.35 (m, 3 H), 3.63-3.79 (m, 3 H), 4.75 (br d, J = 15.2 Hz, 1 H), 6.95 (br d, J = 8.3 Hz, 2 H), 7.09 (br d, J = 8.3 Hz, 2 H), 10.78 (s, 1 H). |
| Int. B-5b | CBM-4 | 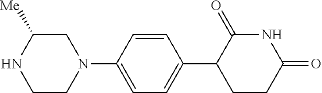 [SMILES: C[C@@H]1CN(c2ccc(C3CCC(=O)NC3=O)cc2)CCN1] | 58% yield, colorless semi-solid [M + H]⁺ = 288.4 ¹H NMR (400 MHz, CHLOROFORM-d) δ ppm 1.29-1.32 (m, 3 H), 1.43-1.46 (m, 9 H), 1.48-1.51 (m, 9 H), 2.09-2.18 (m, 2 H), 2.30-2.47 (m, 2 H), 2.77 (td, J = 11.7, 3.4 Hz, 1 H), 2.97 (dd, J = 12.1, 3.8 Hz, 1 H), 3.26 (td, J = 12.6, 3.4 Hz, 1 H), 3.38 (br d, J = 12.0 Hz, 1 H), 3.52 (br d, J = 12.0 Hz, 1 H), 3.87 (t, J = 7.5 Hz, 1 H), 3.95 (br d, J = 13.2 Hz, 1 H), 4.35 (s, 1 H), 6.88 (d, J = 8.6 Hz, 2 H), 7.22 (d, J = 8.8 Hz, 2 H). |
| Int. B-5c | CBM-6 | 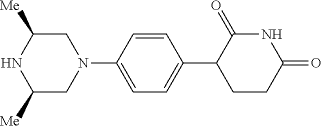 [SMILES: C[C@@H]1CN(c2ccc(C3CCC(=O)NC3=O)cc2)C[C@H](C)N1] | 48% yield, light-yellow semi-solid [M + H]⁺ = 302.2 ¹H NMR (400 MHz, DMSO-d6) δ ppm 1.21 (br d, J = 6.4 Hz, 7 H), 1.96-2.05 (m, 1 H), 2.09-2.22 (m, 1 H), 2.42-2.48 (m, 2 H), 2.58-2.70 (m, 2 H), 3.23-3.28 (m, 1 H), 3.71-3.81 (m, 3 H), 4.70-4.77 (m, 1 H), 6.95 (d, J = 8.8 Hz, 2 H), 7.09 (d, J = 8.6 Hz, 2 H), 8.13-8.15 (m, 1 H), 10.78 (s, 1 H). |
| Int. B-5d | CBM-8 | 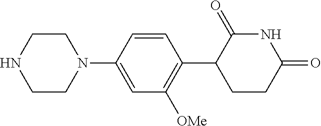 [SMILES: COc1cc(N2CCNCC2)ccc1C1CCC(=O)NC1=O] | 54% yield, tan solid [M + H]⁺ = 304.4 ¹H NMR (400 MHz, DMSO-d6) δ ppm 1.82- 1.92 (m, 1 H), 2.10-2.21 (m, 1 H), 2.40- 2.48 (m, 1 H), 2.59-2.71 (m, 2 H), 3.13- 3.19 (m, 4 H), 3.28-3.31 (m, 4 H), 3.73 (s, 3 H), 3.80 (dd, J = 11.5, 4.9 Hz, 1 H), 6.49 (d, J = 10.8 Hz, 1 H), 6.59 (d, J = 1.7 Hz, 1 H), 6.97 (d, J = 8.3 Hz, 1 H), 10.68 (s, 1 H). |
| Int. B-5e | CBM-13 | 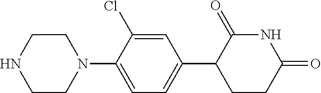 [SMILES: O=C1CCC(c2ccc(N3CCNCC3)c(Cl)c2)C(=O)N1] | 25% yield, light pink solid [M + H]⁺ = 308.2 ¹H NMR (400 MHz, CHLOROFORM-d) δ ppm 2.18-2.30 (m, 1 H), 2.53 (br s, 1 H), 2.69-2.81 (m, 1 H), 3.20-3.26 (m, 4 H), 3.33-3.43 (m, 4 H), 4.08 (dd, J = 12.0, 4.9 Hz, 1 H), 4.77 (br d, J = 15.9 Hz, 1 H), 6.92- 6.98 (m, 1 H), 7.03-7.06 (m, 1 H), 7.18 (br d, J = 8.6 Hz, 1 H), 8.70 (br s, 2 H), 10.84 (s, 1 H) |
| Int. B-5f | CBM-14 | 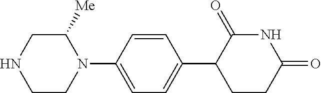 [SMILES: C[C@H]1CNCCN1c1ccc(C2CCC(=O)NC2=O)cc1] | 20% yield, light yellow solid [M + H]⁺ = 288.4 ¹H NMR (400 MHz, DMSO-d6) δ ppm 0.99 (d, J = 6.6 Hz, 3 H), 1.96-2.06 (m, 1 H), 2.08- 2.20 (m, 1 H), 2.41-2.48 (m, 1 H), 2.58- 2.69 (m, 1 H), 2.87-3.05 (m, 3 H), 3.08-3.17 (m, 2 H), 3.21-3.29 (m, 1 H), 3.74 (dd, J = 11.0, 4.9 Hz, 1 H), 3.87-3.97 (m, 1 H), 6.89 (d, J = 8.6 Hz, 2 H), 7.08 (d, J = 8.6 Hz, 2 H), 10.78 (s, 1 H). |
| Int. B-5g | CBM-15 | 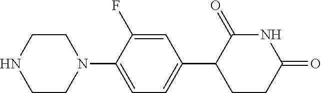 [SMILES: O=C1CCC(c2ccc(N3CCNCC3)c(F)c2)C(=O)N1] | 54% yield, light pink solid [M + H]⁺ = 292.2 ¹H NMR (400 MHz, DMSO-d6) δ ppm 1.79- 1.82 (m, 1 H), 1.93-2.04 (m, 1 H), 2.14- 2.26 (m, 1 H), 2.60-2.71 (m, 1 H), 3.06- 3.19 (m, 6 H), 3.28-3.31 (m, 1 H), 3.82 (br dd, J = 11.9, 4.8 Hz, 1 H), 4.68-4.79 (m, 1 H), 6.91-7.18 (m, 3 H), 10.77-10.89 (m, 1 H) ¹⁹F NMR (377 MHz, DMSO-d6) δ ppm −123.15 (dd, J = 14.3, 8.9 Hz, 1 F). |
| Int. B-5h | CBM-16 | 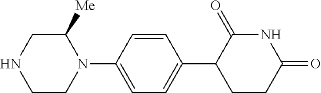 [SMILES: C[C@@H]1CNCCN1c1ccc(C2CCC(=O)NC2=O)cc1] | 47% yield, light yellow solid [M + H]⁺ = 288.2 ¹H NMR (400 MHz, DMSO-d6) δ ppm 1.00 (d, J = 6.6 Hz, 3 H), 1.97-2.07 (m, 1 H), 2.09- 2.19 (m, 1 H), 2.58-2.70 (m, 1 H), 2.92- 3.09 (m, 3 H), 3.14-3.26 (m, 3 H), 3.70- 3.82 (m, 1 H), 3.95 (br d, J = 6.1 Hz, 1 H), 4.69-4.79 (m, 1 H), 6.91 (d, J = 8.6 Hz, 2 H), 7.09 (d, J = 8.6 Hz, 2 H), 10.79 (s, 1 H). |
| Int. B-5i | CBM-17 | 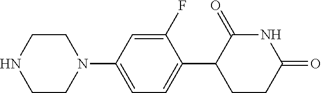 [SMILES: O=C1CCC(c2ccc(N3CCNCC3)cc2F)C(=O)N1] | 76% yield, white solid [M + H]⁺ = 292.4 ¹H NMR (400 MHz, DMSO-d6) δ ppm 1.90- 2.00 (m, 1 H), 2.15 (qd, J = 12.8, 3.9 Hz, 1 H), 2.52 (m, 1 H), 2.65-2.78 (m, 1 H), 3.00- 3.07 (m, 4 H), 3.19-3.26 (m, 4 H), 3.90 (dd, J = 12.2, 4.9 Hz, 1 H), 6.71-6.83 (m, 2 H), 7.08-7.15 (m, 1 H), 10.81 (s, 1 H). ¹⁹F NMR (377 MHz, DMSO-d6) δ ppm- 115.63 (dd, J = 13.6, 9.5 Hz, 1 F). |
| Int. B-5j | CBM-18 | 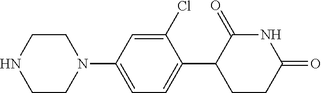 [SMILES: O=C1CCC(c2ccc(N3CCNCC3)cc2Cl)C(=O)N1] | 62% yield, off-white solid [M + H]⁺ = 308.4 ¹H NMR (400 MHz, DMSO-d6) δ ppm 1.90- 1.98 (m, 1 H), 2.23 (qd, J = 12.7, 4.2 Hz, 1 H), 2.68-2.80 (m, 1 H), 2.95-3.03 (m, 4 H), 3.16-3.24 (m, 4 H), 4.06 (dd, J = 12.2, 5.1 Hz, 1 H), 0.00 (d, J = 11.0 Hz, 1 H), 6.97 (d, J = 2.4 Hz, 1 H), 7.14 (d, J = 8.6 Hz, 1 H), 10.83 (s, 1 H). |
| Int. B-5k | CBM-20 | 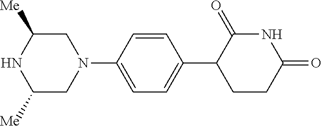 [SMILES: C[C@H]1CN(c2ccc(C3CCC(=O)NC3=O)cc2)C[C@H](C)N1] | 48% yield, tan solid [M + H]⁺ = 302.4 ¹H NMR (400 MHz, DMSO-d6) δ ppm 1.23 (d, J = 6.6 Hz, 6 H), 1.95-2.05 (m, 1 H), 2.07- 2.22 (m, 1 H), 2.42-2.48 (m, 1 H), 2.57- 2.70 (m, 1 H), 2.93 (dd, J = 12.2, 6.4 Hz, 2 H), 3.26 (br dd, J = 12.3, 3.1 Hz, 3 H), 3.41- 3.52 (m, 3 H), 3.73 (dd, J = 11.2, 4.9 Hz, 1 H), 4.70-4.77 (m, 1 H), 6.82-6.98 (m, 2 H), 7.06 (d, J = 8.8 Hz, 2 H), 10.77 (s, 1 H). |
| Int. B-51 | CBM-21 | 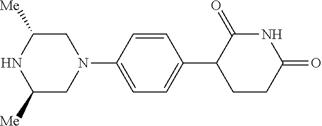 [SMILES: C[C@@H]1CN(c2ccc(C3CCC(=O)NC3=O)cc2)C[C@@H](C)N1] | 76% yield, light yellow solid [M + H]⁺ = 302.4 ¹H NMR (400 MHz, DMSO-d6) δ ppm 1.23 (s, 3 H), 1.25 (s, 3 H), 1.93-2.04 (m, 1 H), 2.09-2.19 (m, 1 H), 2.64 (ddd, J = 17.1, 11.5, 5.1 Hz, 1 H), 2.96 (dd, J = 12.3, 6.2 Hz, 2 H), 3.25-3.29 (m, 2 H), 3.46-3.56 (m, 2 H), 3.74 (dd, J = 11.2, 4.9 Hz, 1 H), 4.66-4.81 (m, 1 H), 6.91 (d, J = 8.6 Hz, 2 H), 7.07 (d, J = 8.8 Hz, 2 H), 10.73-10.80 (m, 1 H). |

### Example S3. General Procedure for CBM-C

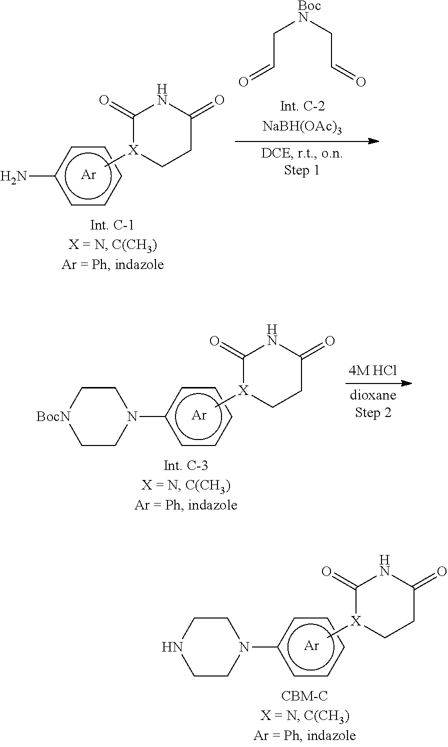

### Synthesis of CBM-C: Step 1

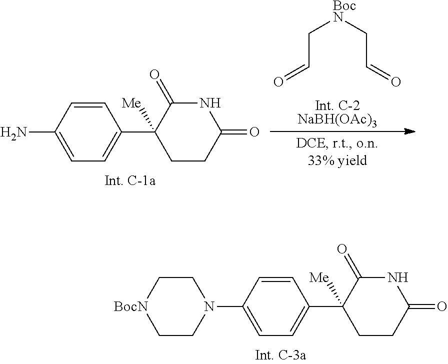 [SMILES: C[C@@]1(c2ccc(N)cc2)CCC(=O)NC1=O ; CC(C)(C)OC(=O)N(CC=O)CC=O ; CC(C)(C)OC(=O)N1CCN(c2ccc([C@]3(C)CCC(=O)NC3=O)cc2)CC1 ; "NaBH(OAc)3"]

To a solution of tert-butyl N,N-bis(2-oxoethyl)carbamate Int. C-2 (553.18 mg, 2.75 mmol) in DCE (20 mL) was added (3 S)-3-(4-aminophenyl)-3-methyl-piperidine-2,6-di one Int. C-la (500 mg, 2.29 mmol) under N₂. After 5 minutes, sodium triacetoxyborohydride (1165.42 mg, 5.5 mmol) was added. The reaction was stirred at room temperature for 24 h. HPLC and LCMS showed incomplete conversion of starting material. Therefore, sodium triacetoxyborohydride (1165.42 mg, 5.5 mmol) was added at room temperature and the reaction was left for a further 24 h. HPLC and LCMS showed complete conversion of starting material. The reaction mixture was partitioned between DCM and sat. NaHCO₃. The organic phase was extracted with DCM (2×60 mL). The combined organics were washed with brine, dried over MgSO₄ and concentrated to give crude product, which was purified by reverse phase column chromatography (500 to 100% MeCN in water w/0.1% formic acid), affording tert-butyl (S)-4-(4-(3-methyl-2,6-dioxopiperidin-3-yl)phenyl)piperazine-1-carboxylate Int. C-3a (300 mg, 33.7% yield) as an off-white solid.

LCMS: [M+H]⁺=388.2.

¹H NMR (400 MHz, DMSO-d₆) δ ppm 1.41 (s, 9H), 2.00-2.10 (m, 2H), 2.27-2.33 (m, 1H), 2.39-2.45 (m, 1H), 3.05-3.12 (m, 3H), 3.32 (s, 4H), 3.41-3.49 (m, 4H), 6.94 (d, J=8.8 Hz, 2H), 7.12 (d, J=8.8 Hz, 2H), 10.85 (s, 1H).

TABLE 13
List of Int. C-1 compounds used in CBM-C: Step 1
Starting Material

| Int C. | Structure | Reference |
|---|---|---|
| Int. C-1a | 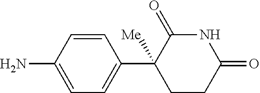 [SMILES: C[C@@]1(c2ccc(N)cc2)CCC(=O)NC1=O] | Procedure provided below |
| Int. C-1b | 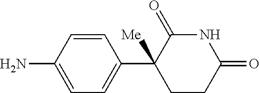 [SMILES: C[C@]1(c2ccc(N)cc2)CCC(=O)NC1=O] | Procedure provided below |
| Int. C-1c | 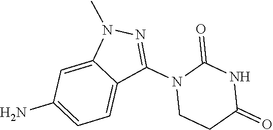 [SMILES: Cn1nc(N2CCC(=O)NC2=O)c2ccc(N)cc21] | Procedure provided below |
| Int. C-1d | 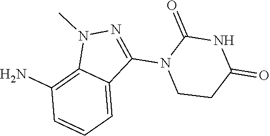 [SMILES: Cn1nc(N2CCC(=O)NC2=O)c2cccc(N)c21] | Previously reported in: WO 2021/ 262815 A1 |

TABLE 14
List of Int. C-3 compounds generated by General Procedure CBM-C: Step 1

| Starting | | | |
|---|---|---|---|
| Material | Product | | |
| Int C.1 | Int C.3 | Structure | Characterization |
| Int. C-1a | Int. C-3a | 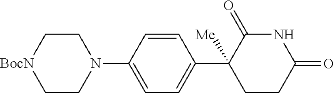 [SMILES: CC(C)(C)OC(=O)N1CCN(c2ccc([C@]3(C)CCC(=O)NC3=O)cc2)CC1] | 34% yield, off-white solid [M + H]⁺ = 388.2 ¹H NMR (400 MHz, DMSO-d6) δ ppm 1.41 (s, 9 H), 2.00-2.10 (m, 2 H), 2.27-2.33 (m, 1 H), 2.39-2.45 (m, 1 H), 3.05-3.12 (m, 3 H), 3.32 (s, 4 H), 3.41-3.49 (m, 4 H), 6.94 (d, J = 8.8 Hz, 2 H), 7.12 (d, J = 8.8 Hz, 2 H), 10.85 (s, 1 H). |
| Int. C-1b | Int. C-3b | 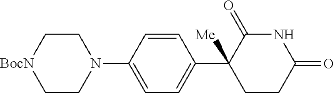 [SMILES: CC(C)(C)OC(=O)N1CCN(c2ccc([C@@]3(C)CCC(=O)NC3=O)cc2)CC1] | 65% yield, off-white solid [M + H]⁺= 388.2 ¹H NMR (400 MHz, DMSO-d6) δ ppm 1.38 (s, 3 H), 1.41 (s, 9 H), 2.02-2.10 (m, 2 H), 2.29- 2.34 (m, 1 H), 2.39-2.44 (m, 1 H), 3.06-3.10 (m, 4 H), 3.42-3.46 (m, 4 H), 6.94 (d, J = 8.8 Hz, 2 H), 7.12 (d, J = 8.8 Hz, 2 H), 10.84 (s, 1 H) |
| Int. C-1c | Int. C-3c | 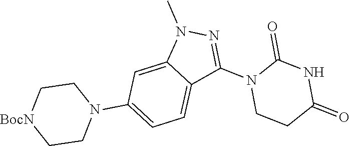 [SMILES: Cn1nc(N2CCC(=O)NC2=O)c2ccc(N3CCN(C(=O)OC(C)(C)C)CC3)cc21] | 34% yield, off-white solid [M + H]⁺ = 429.2 ¹H NMR (400 MHz, DMSO-d6) δ ppm 1.43 (s, 9 H), 2.73 (t, J = 6.6 Hz, 2 H), 3.16-3.24 (m, 4 H), 3.45-3.54 (m, 4 H), 3.85-3.95 (m, 5 H), 6.87 (d, J = 1.7 Hz, 1 H), 6.93 (dd, J = 9.0, 2.0 Hz, 1 H), 7.44-7.53 (m, 1 H), 10.50 (s, 1 H). |
| Int. C-1d | Int. C-3d | 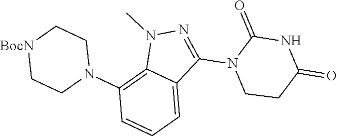 [SMILES: Cn1nc(N2CCC(=O)NC2=O)c2cccc(N3CCN(C(=O)OC(C)(C)C)CC3)c21] | 42% yield, off-white solid [M + H]⁺ = 429.2 ¹H NMR (400 MHz, DMSO-d6) δ ppm 1.43 (s, 9 H), 2.36 (s, 3 H), 2.79 (t, J = 6.6 Hz, 2 H), 3.20 (br d, J = 4.4 Hz, 4 H), 3.53 (br d, J = 4.9 Hz, 4 H), 3.68-3.77 (m, 2 H), 6.77 (dd, J = 7.8, 1.0 Hz, 1 H), 7.04-7.08 (m, 1 H), 7.09-7.14 (m, 1 H), 10.45 (s, 1 H). |

### Synthesis of (S)-3-(4-aminophenyl)-3-methylpiperidine-2,6-dione (C-la) and (R)-3-(4-aminophenyl)-3-methylpiperidine-2,6-dione (C-1b)

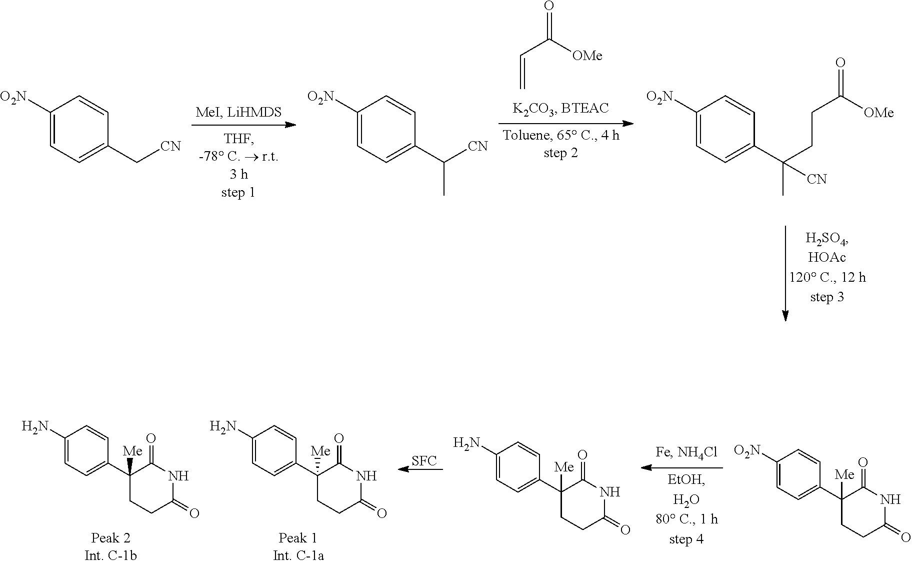

Step 1: 2-(4-Nitrophenyl)propanenitrile: To a solution of 2-(4-nitrophenyl)acetonitrile (90 g, 555 mmol) in dry THF (2500 mL) under nitrogen atmosphere at −78° C. was added LiHMDS (666 mL, 666 mmol, 1M in THF). The mixture was stirred for 1 h at −78° C. followed by addition of iodomethane (86.7 g, 611 mmol). After stirring at −78° C. for 1 h, the reaction mixture was allowed to warm to room temperature and stirred for 2 h. The reaction mixture was cooled to 0° C., and quenched with citric acid (1000 mL, 10%) dropwise at 0° C., and then extracted with ethyl acetate (1000 mL×2), the combined organic layers were washed with brine (1500 mL), dried over anhydrous Na₂SO₄, filtered and concentrated. The residue was purified by column chromatography (petroleum ether/ethyl acetate, 1:1 to 10:1) affording 2-(4-nitrophenyl)propanenitrile (36.8 g, 37.6% yield) as a yellow solid.

¹H NMR (400 MHz, CDCl₃) δ: 8.25 (d, J=8.4 Hz, 2H), 7.55 (d, J=8.4 Hz, 2H), 4.05-3.99 (m, 2H), 1.68 (d, J=4.8 Hz, 3H).

Step 2: Methyl 4-cyano-4-(4-nitrophenyl)pentanoate: To a solution of 2-(4-nitrophenyl)propanenitrile (65 g, 369 mmol) in toluene (650 mL) were added methyl prop-2-enoate (63.5 g, 738 mmol), K₂CO₃ (102 g, 738 mmol) and BTEAC (16.8 g, 73.8 mmol). The reaction was stirred at 65° C. for 4 h. The reaction was filtered, and the filtrate was concentrated to give methyl 4-cyano-4-(4-nitrophenyl)pentanoate (75 g, crude) as a yellow solid, which was used directly without further purification.

Step 3: 3-Methyl-3-(4-nitrophenyl)piperidine-2,6-dione: To a solution of methyl 4-cyano-4-(4-nitrophenyl)pentanoate (75 g, 286 mmol) in acetic acid (700 mL) was added H₂SO₄ (2.8 g, 28.6 mmol), then the reaction was stirred at 120° C. for 12 h. The reaction mixture was cooled to room temperature and concentrated, the residue was washed with MTBE (100 mL) and dried to give crude 3-methyl-3-(4-nitrophenyl) piperidine-2,6-dione (72 g, crude) as a gray solid, which was used directly without further purification.

Step 4: rac-3-(4-Aminophenyl)-3-methylpiperidine-2,6-dione: To a solution of 3-methyl-3-(4-nitrophenyl)piperidine-2,6-dione (72 g, 290 mmol) in ethanol (720 mL) was added Fe (81 g, 1450 mmol), NH₄Cl (155 g, 2900 mmol) and water (360 mL), then the reaction was stirred at 80° C. for 1 h. The reaction mixture was cooled to room temperature and filtered, and the filtrate was concentrated. The residue was extracted with ethyl acetate (400 mL×3), and the combined organic layers were washed with brine (600 mL), dried over anhydrous Na₂SO₄, filtered, and concentrated to give 3-(4-aminophenyl)-3-methyl-piperidine-2,6-dione (50.2 g, 79.3% yield) as a gray solid.

¹H NMR (400 MHz, CDCl₃) δ: 8.0 (br s, 1H), 7.04-7.01 (m, 1H), 6.66 (t, J=2.0 Hz, 1H), 3.73 (br s, 2H), 2.56-2.41 (m, 1H), 2.38-2.33 (m, 2H), 2.32-2.08 (m 1H), 1.53 (s, 3H).

SFC: (S)-3-(4-Aminophenyl)-3-methylpiperidine-2,6-dione (Int. C-1a) and (R)-3-(4-Aminophenyl)-3-methylpiperidine-2,6-dione (Int. C-1b): Separation Conditions: Instrument—Thar SFC350 preparative SFC; Column—Daicel Chiralcel OJ, 250×50 mm, i.d. 10 μm; Mobile Phase—A for CO₂, B for IPA w/0.1% ammonium hydroxide; Gradient—B %=60%; Flow Rate—200 g/min; Wavelength—220 nM; Column Temperature—40° C.; System Back Pressure—100 bar. 55 g of rac-3-(4-aminophenyl)-3-methylpiperidine-2,6-dione was separated into 25.9 g of (S)-3-(4-aminophenyl)-3-methylpiperidine-2,6-dione (Int. C-1a) [peak 1] and 26.5 g of (R)-3-(4-aminophenyl)-3-methylpiperidine-2,6-dione (Int. C-1b) [peak 2].

### Synthesis of 1-(6-Amino-1-methyl-1H-indazol-3-yl)dihydropyrimidine-2,4(1H,3H)-dione (C-1c)

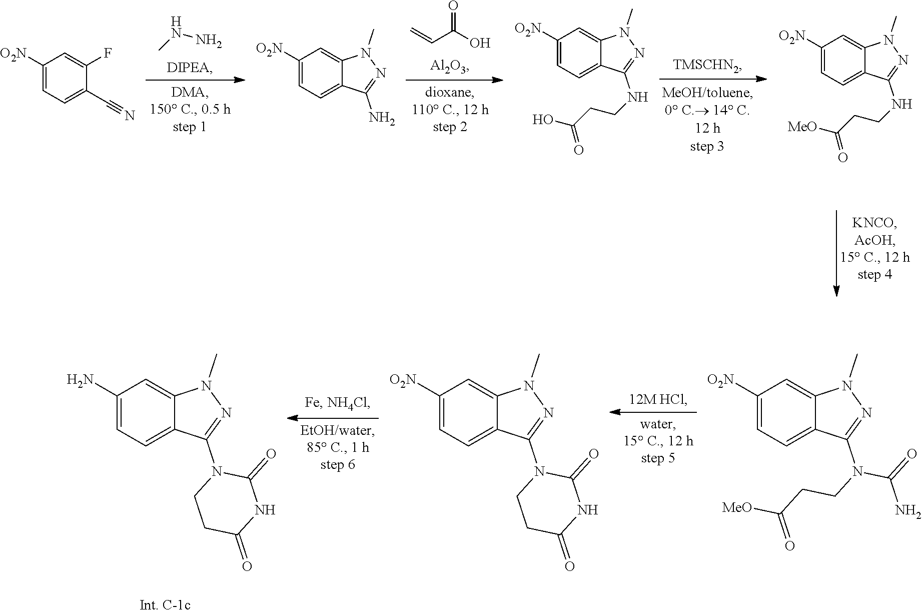

Step 1: 1-Methyl-6-nitro-1H-indazol-3-amine: To a solution of 2-fluoro-4-nitro-benzonitrile (5.0 g, 30.1 mmol, 1.0 eq) in dimethylacetamide (25.0 mL) was added methylhydrazine (10.4 g, 90.3 mmol, 11.9 mL, 3.0 eq), N-ethyl-N-isopropylpropan-2-amine (4.28 g, 33.1 mmol, 5.8 mL, 1.1 eq) and dimethylacetamide (25.0 mL). The mixture was stirred at 150° C. for 0.5 h. The mixture was diluted with petroleum ether (300 mL) and ethyl acetate (30 mL). The mixed solution was stirred for 1 h and filtered to give a residue. 1-Methyl-6-nitro-indazol-3-amine (7.0 g, crude) was obtained as brown solid and used directly without further purification.

LCMS: [M+H]⁺: 193.1.

¹H NMR (400 MHz, DMSO-d₆): δ 8.35 (s, 1H), 7.90 (d, J=8.8 Hz, 1H), 7.69 (d, J=8.4 Hz, 1H), 5.78 (s, 2H), 3.86 (s, 3H).

Step 2: 3-((1-Methyl-6-nitro-1H-indazol-3-yl)amino)propanoic acid: To 1-methyl-6-nitro-indazol-3-amine (1.2 g, 6.24 mmol, 1.0 eq) was added aluminum oxide (1.91 g, 18.73 mmol, 3.0 eq), dioxane (5.0 mL) and acrylic acid (0.9 g, 12.49 mmol, 0.9 mL, 2.0 eq). The mixture was stirred at 110° C. for 12 h. The mixture was diluted with dichloromethane (100 mL), filtered, and concentrated to give to afford 3-((1-methyl-6-nitro-1H-indazol-3-yl)amino)propanoic acid (15 g, crude) was obtained as brown oil and used without further purification.

LCMS: [M+H]⁺: 265.0.

Step 3: Methyl 3-((1-methyl-6-nitro-1H-indazol-3-yl)amino)propanoate: To a solution of 3-((1-methyl-6-nitro-1H-indazol-3-yl)amino)propanoic acid (15.0 g, 56.77 mmol, 1.0 eq) in methanol (70 mL) and toluene (70 mL) was added trimethyl silicon diazomethane (2 M, 85.2 mL, 3.0 eq) at 0° C. The mixture was stirred at 15° C. for 12 h. The reaction mixture was concentrated to give a residue. The residue was purified by silica gel column chromatography (1% to 20% EtOAc in petroleum ether), affording methyl 3-((1-methyl-6-nitro-1H-indazol-3-yl)amino)propanoate (4.5 g, crude) as brown solid.

Step 4: Methyl 3-(1-(1-methyl-6-nitro-1H-indazol-3-yl)ureido)propanoate: To a solution of methyl 3-((1-methyl-6-nitro-1H-indazol-3-yl)amino)propanoate (4.5 g, 16.17 mmol, 1.00 eq) in acetic acid (50 mL) was added potassium cyanate (6.89 g, 80.86 mmol, 5.0 eq). The mixture was stirred at 15° C. for 12 h. The mixture was concentrated, diluted with water (150 mL), and extracted with ethyl acetate (3×150 mL). The combined organic layers were washed with brine (150 mL), dried over sodium sulfate, filtered, and concentrated under reduced pressure to afford methyl 3-(1-(1-methyl-6-nitro-1H-indazol-3-yl)ureido)propanoate (6.100 g, crude) as brown oil, which was used without further purification.

LCMS: [M+H]⁺: 322.6.

Step 5: 1-(1-Methyl-6-nitro-1H-indazol-3-yl)dihydropyrimidine-2,4(1H,3H)-dione: To methyl 3-(1-(1-methyl-6-nitro-1H-indazol-3-yl)ureido)propanoate (6.0 g, 18.67 mmol, 1.0 eq) was added HCl (12 M, 50.0 mL, 32.13 eq) at 0° C. The mixture was stirred at 15° C. for 12 h. Water (100 mL) was added to the reaction mixture. The mixture was concentrated and lyophilized to afford a residue. The residue was purified by semi-preparative reverse phase-HPLC (23% to 48% MeCN in water w/0.05% ammonium hydroxide), affording 1-(1-methyl-6-nitro-indazol-3-yl)hexahydropyrimidine-2,4-dione (1.0 g, 3.46 mmol, 18.5% yield) as a yellow solid.

LCMS: [M+H]⁺: 290.0.

¹H NMR (400 MHz, DMSO-d₆): δ 10.66 (s, 1H), 8.70 (d, J=1.2 Hz, 1H), 8.05-7.88 (m, 2H), 4.14 (s, 3H), 3.98 (t, J=6.8 Hz, 2H), 2.78 (t, J=6.8 Hz, 2H).

Step 6: 1-(6-Amino-1-methyl-1H-indazol-3-yl)dihydropyrimidine-2,4(1H,3H)-dione (Int. C-1c): To a solution of 1-(1-methyl-6-nitro-indazol-3-yl)hexahydropyrimidine-2,4-dione (1.0 g, 3.46 mmol, 1.0 eq) in ethanol (40 mL) and water (20 mL) was added iron (0.965 g, 17.29 mmol, 5.0 eq) and ammonium chloride (1.85 g, 34.57 mmol, 10.0 eq). The mixture was stirred at 85° C. for 1 h. The mixture was filtered and concentrated to give a residue which was purified by silica gel column chromatography (1% to 5% MeOH in DCM), affording 1-(6-amino-1-methyl-indazol-3-yl)hexahydropyrimidine-2,4-dione (0.599 g, 2.31 mmol, 67% yield, 100% purity) as white solid.

LCMS: [M+H]⁺: 260.0.

¹H NMR (400 MHz, DMSO-d₆): δ 10.48 (s, 1H), 7.27 (d, J=8.8 Hz, 1H), 6.47 (dd, J=1.6, 8.8 Hz, 1H), 6.39 (d, J=1.2 Hz, 1H), 5.41 (s, 2H), 3.85 (t, J=6.8 Hz, 2H), 3.80-3.72 (m, 3H), 2.71 (t, J=6.8 Hz, 2H).

### Synthesis of CBM-C: Step 2

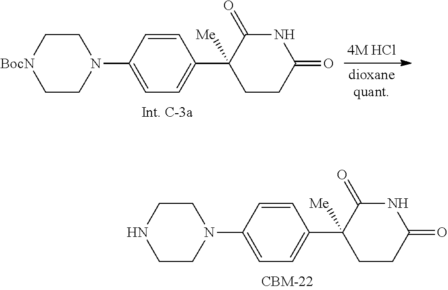 [SMILES: CC(C)(C)OC(=O)N1CCN(c2ccc([C@]3(C)CCC(=O)NC3=O)cc2)CC1 ; C[C@@]1(c2ccc(N3CCNCC3)cc2)CCC(=O)NC1=O]

To a solution of tert-butyl 4-[4-(3S)-3-methyl-2,6-dioxo-3-piperidyl]phenyl]piperazine-1-carboxylate Int. C-3a (300 mg, 0.7700 mmol) in 1,4-dioxane (5 mL) was added 4 M HCl in dioxane (3.88 mL, 15.52 mmol) at room temperature. The reaction was stirred at room temperature for 5 h. HPLC and LCMS showed complete conversion of starting material to the desired product. The reaction mixture was concentrated to dryness by chasing with acetonitrile affording the desired bis-hydrochloride salt of (S)-3-methyl-3-(4-(piperazin-1-yl)phenyl)piperidine-2,6-dione CBM-22 (280 mg, quant.) as a white solid.

LCMS: [M+H]⁺=288.2

¹H NMR (400 MHz, DMSO-d₆) δ ppm 1.39 (s, 3H), 2.02-2.12 (m, 2H), 2.29-2.37 (m, 1H), 2.40-2.45 (m, 1H), 3.19 (br s, 4H), 3.32-3.40 (m, 4H), 6.98 (d, J=8.8 Hz, 2H), 7.16 (d, J=8.8 Hz, 2H), 9.20 (br s, 3H), 10.86 (s, 1H).

TABLE 15
List of CBM-C compounds generated by General Procedure CBM-C: Step 2

| Starting | | | |
|---|---|---|---|
| Material | Product | | |
| Int C.1 | Int C.3 | Structure | Characterization |
| Int. C-1a | Int. C-3a | 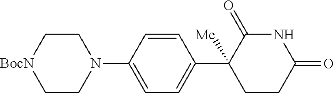 [SMILES: CC(C)(C)OC(=O)N1CCN(c2ccc([C@]3(C)CCC(=O)NC3=O)cc2)CC1] | 34% yield, off-white solid [M + H]⁺ = 388.2 ¹H NMR (400 MHz, DMSO-d₆) δ ppm 1.41 (s, 9 H), 2.00-2.10 (m, 2 H), 2.27-2.33 (m, 1 H), 2.39-2.45 (m, 1 H), 3.05-3.12 (m, 3 H), 3.32 (s, 4 H), 3.41-3.49 (m, 4 H), 6.94 (d, J = 8.8 Hz, 2 H), 7.12 (d, J = 8.8 Hz, 2 H), 10.85 (s, 1 H). |
| Int. C-1b | Int. C-3b | 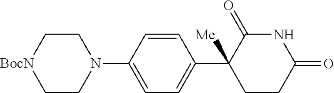 [SMILES: CC(C)(C)OC(=O)N1CCN(c2ccc([C@@]3(C)CCC(=O)NC3=O)cc2)CC1] | 65% yield, off-white solid [M + H]⁺= 388.2 ¹H NMR (400 MHz, DMSO-d₆) δ ppm 1.38 (s, 3 H), 1.41 (s, 9 H), 2.02-2.10 (m, 2 H), 2.29-2.34 (m, 1 H), 2.39-2.44 (m, 1 H), 3.06-3.10 (m, 4 H), 3.42-3.46 (m, 4 H), 6.94 (d, J = 8.8 Hz, 2 H), 7.12 (d, J = 8.8 Hz, 2 H), 10.84 (s, 1 H). |
| Int. C-1c | Int. C-3c | 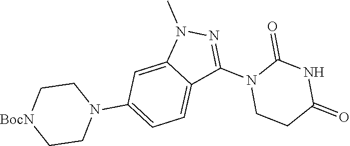 [SMILES: Cn1nc(N2CCC(=O)NC2=O)c2ccc(N3CCN(C(=O)OC(C)(C)C)CC3)cc21] | 34% yield, off-white solid [M + H]⁺ = 429.2 ¹H NMR (400 MHz, DMSO-d₆) δ ppm 1.43 (s, 9 H), 2.73 (t, J = 6.6 Hz, 2 H), 3.16-3.24 (m, 4 H), 3.45-3.54 (m, 4 H), 3.85-3.95 (m, 5 H), 6.87 (d, J = 1.7 Hz, 1 H), 6.93 (dd, J = 9.0, 2.0 Hz, 1 H), 7.44-7.53 (m, 1 H), 10.50 (s, 1 H). |
| Int. C-1d | Int. C-3d | 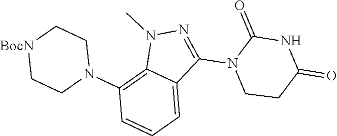 [SMILES: Cn1nc(N2CCC(=O)NC2=O)c2cccc(N3CCN(C(=O)OC(C)(C)C)CC3)c21] | 42% yield, off-white solid [M + H]⁺ = 429.2 ¹H NMR (400 MHz, DMSO-d₆) δ ppm 1.43 (s, 9 H), 2.36 (s, 3 H), 2.79 (t, J = 6.6 Hz, 2 H), 3.20 (br d, J = 4.4 Hz, 4 H), 3.53 (br d, J = 4.9 Hz, 4 H), 3.68-3.77 (m, 2 H), 6.77 (dd, J = 7.8, 1.0 Hz, 1 H), 7.04-7.08 (m, 1 H), 7.09-7.14 (m, 1 H), 10.45 (s, 1 H). |

### Example S4. Procedure for CBM-5

### Synthesis of 3-(1-oxo-5-(piperazin-1-yl)isoindolin-2-yl)piperidine-2,6-dione (CBM-5)

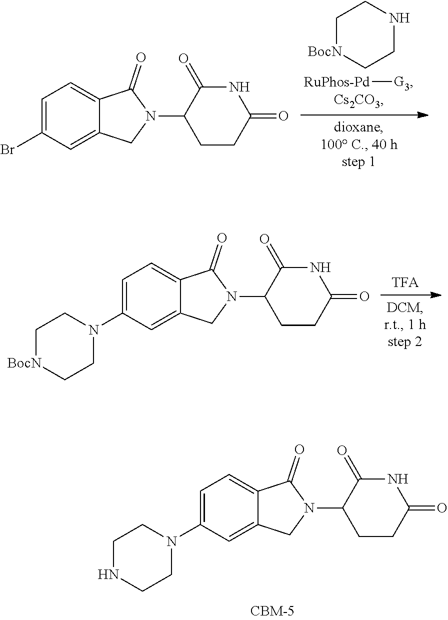

Step 1: tert-Butyl 4-(2-(2,6-dioxopiperidin-3-yl)-1-oxoisoindolin-5-yl)piperazine-1-carboxylate: A round-bottom flask was charged with 3-(5-Bromo-1-oxoisoindolin-2-yl)piperidine-2,6-dione (5.0 g, 15.47 mmol, 1 eq), tert-butyl piperazine-1-carboxylate (3.17 g, 17.02 mmol, 1.1 eq), methanesulfonato(2-dicyclohexylphosphino-2′,6′-di-1-propoxy-1,1′-biphenyl)(2′-amino-1,1′-biphenyl-2-yl)palladium(II) (0.647 g, 0.77 mmol, 0.05 eq) and cesium carbonate (7.56 g, 23.21 mmol, 1.5 eq) and dioxane (100.00 mL). The reaction vessel was purged with nitrogen and heated at 110° C. for 40 hours. The reaction mixture was filtered, and the filter cake was washed with dichloromethane (50 mL). The combined organics were concentrated under vacuum to afford a residue which was purified by silica gel column chromatography (000 to 100% EtOAc in petroleum ether). The resulting material was triturated in EtOAc/petroleum ether (2 mL/2 mL) and filtered. The filter cake was dried in vacuum to afford the product tert-butyl 4-(2-(2,6-dioxopiperidin-3-yl)-1-oxoisoindolin-5-yl)piperazine-1-carboxylate (0.300 g, yield: 4.520%) as a white solid.

LCMS: [M+H]⁺=429.1.

¹HNMR (400 MHz, CDCl₃): δ 8.04 (br s, 1H), 7.76 (d, J=11.6 Hz, 1H), 7.00 (dd, J=11.6, 2.4 Hz, 1H), 6.89 (s, 1H), 5.21 (dd, J=17.6, 6.8 Hz, 1H), 4.48-4.21 (m, 2H), 3.67-3.53 (m, 4H), 3.35-3.20 (m, 4H), 2.98-2.76 (m, 2H), 2.42-2.15 (m, 2H), 1.50 (s, 9H).

Step 2: 3-(1-Oxo-5-(piperazin-1-yl)isoindolin-2-yl)piperidine-2,6-dione (CBM-5): To a solution of tert-butyl 4-(2-(2,6-dioxopiperidin-3-yl)-1-oxoisoindolin-5-yl)piperazine-1-carboxylate (0.150 g, 0.35 mmol, 1 eq) in dichloromethane (2.00 mL) was added trifluoroacetic acid (0.798 g, 7.00 mmol, 0.52 mL, 20 eq) in one portion. The mixture was then stirred at 25° C. for about 1 hour. The solvent was removed in vacuum to give a residue and partitioned between water (5.00 mL) and dichloromethane (3.00 mL). The aqueous phase was washed with dichloromethane (3.00 mL×2), and the aqueous layer was concentrated in high pressure at 45° C. with water bath to afford the product 3-(1-oxo-5-(piperazin-1-yl)isoindolin-2-yl)piperidine-2,6-dione (CBM-5) (64.43 mg, 42% yield, 98.9% purity, TFA salt) as a light red gum.

LCMS: [M+H]⁺=329.1.

¹HNMR (400 MHz, DMSO-d₆) 10.97 (s, 1H), 8.89 (brs, 2H), 7.58 (d, J=8.4 Hz, 1H), 7.21-7.06 (m, 2H), 5.06 (dd, J=13.6, 5.2 Hz, 1H), 4.43-4.17 (m, 2H), 3.59-3.44 (m, 4H), 3.31-3.20 (m, 4H), 2.98-2.84 (m, 1H), 2.61-2.56 (m, 1H), 2.43-2.32 (m, 1H), 2.02-1.91 (m, 1H).

### Example S5. Procedure for CBM-7

### Synthesis of 3-(1-oxo-6-(piperazin-1-yl)isoindolin-2-yl)piperidine-2,6-dione (CBM-7)

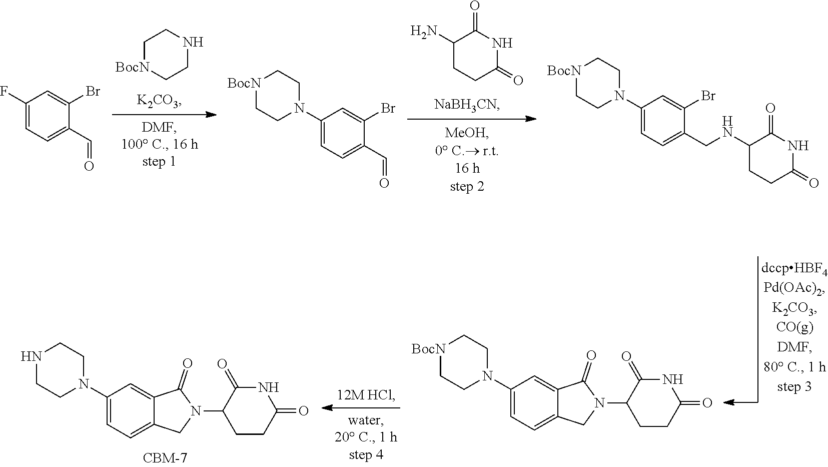

Step 1: tert-Butyl 4-(3-bromo-4-formylphenyl)piperazine-1-carboxylate: To a solution of 2-bromo-4-fluoro-benzaldehyde (8 g, 39.4 mmol) and tert-butyl piperazine-1-carboxylate (8.81 g, 47.3 mmol) in DMF (80 mL) was added K₂CO₃ (10.9 g, 78.8 mmol). The reaction mixture was stirred at 100° C. for 16 hours. The reaction mixture was cooled to rt and diluted with EtOAc (200 mL) and water (200 mL), the aqueous layer was extracted with EtOAc (100 mL×2), the combined organic layers were washed with brine (400 mL), dried over anhydrous Na₂SO₄, filtered and concentrated. The resulting residue was purified by silica gel column chromatography (10% to 50% EtOAc in petroleum ether) affording the product tert-butyl 4-(3-bromo-4-formyl-phenyl)piperazine-1-carboxylate (11.8 g, 81.1% yield) as an off-white solid.

¹H NMR: (400 MHz, DMSO-d₆) δ: 9.96 (s, 1H), 7.68 (d, J=8.8 Hz, 1H), 7.16 (s, 1H), 7.04-7.01 (m, 1H), 3.45 (s, 8H), 1.43 (s, 9H).

Step 2: tert-Butyl 4-(3-bromo-4-(((2,6-dioxopiperidin-3-yl)amino)methyl)phenyl)piperazine-1-carboxylate: To a stirred solution of 3-aminopiperidine-2,6-dione (3.92 g, 23.8 mmol, HCl salt) in MeOH (250 mL) was added NH₃ in MeOH solution until pH=7. The reaction was allowed to stir at 20° C. Then, AcOH was added dropwise until pH=6. At this point, tert-butyl 4-(3-bromo-4-formyl-phenyl)piperazine-1-carboxylate (8.8 g, 23.8 mmol) was added and stirred at 20° C. for 10 min. After that, NaBH₃CN (4.49 g, 71.5 mmol) was added at 0° C. The reaction mixture was stirred at 20° C. for 16 h. The reaction mixture was concentrated, the residue was filtered the cake was washed with petroleum ether/EtOAc (5:1, 50 mL) to give tert-butyl 4-[3-bromo-4-[[(2,6-dioxo-3-piperidyl)amino]methyl]phenyl]piperazine-1-carboxylate (7.5 g, 65.4% yield) as a white solid.

¹H NMR: (400 MHz, DMSO-d₆) δ: 10.74 (s, 1H), 7.34 (d, J=8.8 Hz, 1H), 7.12 (d, J=2.0 Hz, 1H), 6.97-6.95 (m, 1H), 3.78 (d, J=6.0 Hz, 1H), 3.42 (t, J=4.8 Hz, 4H), 3.11 (t, J=5.2 Hz, 4H), 2.73-2.68 (m, 1H), 2.56-2.54 (m, 1H), 2.13-2.10 (m 1H), 1.42 (s, 9H).

Step 3: tert-Butyl 4-(2-(2,6-dioxopiperidin-3-yl)-3-oxoisoindolin-5-yl)piperazine-1-carboxylate: To a solution of tert-butyl 4-[3-bromo-4-[[(2,6-dioxo-3-piperidyl)amino]methyl]phenyl]piperazine-1-carboxylate (5 g, 10.4 mmol) and dicyclohexyl(3-dicyclohexylphosphaniumyl propyl)phosphonium ditetrafluoroborate (636 mg, 1.04 mmol) in DMF (50 mL) was added diacetoxypalladium (233 mg, 1.04 mmol) and K₂CO₃ (2.15 g, 15.6 mmol) under nitrogen. The suspension was degassed under vacuum and purged with CO several times. The mixture was stirred under CO (50 psi) at 80° C. for 48 h. The reaction mixture was cooled to rt and filtered. The filtrate was concentrated, and the residue was purified by preparative (20% to 50% MeCN in water w/0.1% TFA) to give tert-butyl 4-[2-(2,6-dioxo-3-piperidyl)-3-oxo-isoindolin-5-yl]piperazine-1-carboxylate (3 g, 67.4% yield) as a gray solid.

LCMS: [M+H]⁺=429.1.

Step 4: 3-(1-Oxo-6-(piperazin-1-yl)isoindolin-2-yl)piperidine-2,6-dione (CBM-7): tert-Butyl 4-[2-(2,6-dioxo-3-piperidyl)-3-oxo-isoindolin-5-yl]piperazine-1-carboxylate (5 g, 11.7 mmol) was added to HCl (12 N, 15 mL) at 0° C. The reaction mixture was stirred at 20° C. for 1 h. The reaction mixture was diluted with MeCN at 0-10° C. The resulting precipitate was filtered, and the cake was dried to give 3-(1-oxo-6-piperazin-1-yl-isoindolin-2-yl)piperidine-2,6-dione (4.3 g, HCl salt, yield 100%) as a gray solid.

¹H NMR: (400 MHz, DMSO-d₆) δ: 10.98 (s, 1H), 9.34 (s, 2H), 7.49 (d, J=5.2 Hz, 1H), 7.33-7.27 (m, 2H), 5.13-5.09 (m, 1H), 4.30 (dd, J=17.2 Hz, J=58.2 Hz, 2H), 3.46 (d, J=4.8 Hz, 4H), 3.23 (d, J=4.8 Hz, 1H), 2.93-2.78 (m, 1H), 2.62-2.51 (m, 1H), 2.48-2.37 (m, 1H), 2.01-1.98 (m, 1H).

### Example S6. Procedure for CBM-10

### Synthesis of 3-(1-Methyl-6-(piperazin-1-yl)-1H-indazol-3-yl)piperidine-2,6-dione (CBM-10)

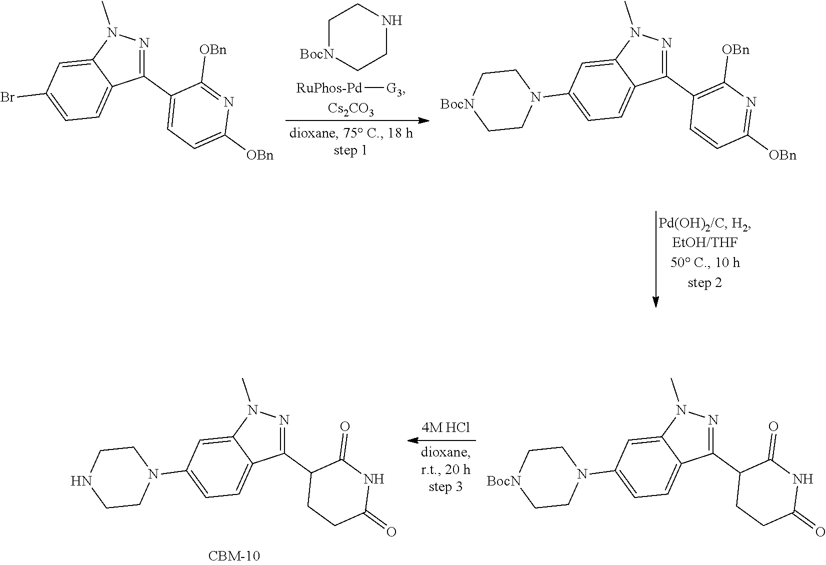

Step 1: tert-Butyl 4-(3-(2,6-Bis(benzyloxy)pyridin-3-yl)-1-methyl-1H-indazol-6-yl)piperazine-1-carboxylate: A mixture of 6-bromo-3-(2,6-dibenzyloxy-3-pyridyl)-1-methyl-indazole (30.0 g, 60.0 mmol), tert-butyl piperazine-1-carboxylate (16.8 g, 89.9 mmol), RuPhos-Pd-G3 (10 g, 12.0 mmol) and Cs₂CO₃ (23.4 g, 71.9 mmol) in degassed 1,4-dioxane (150 mL) was heated to 75° C. for 18 h and then cooled to rt. The mixture was filtered through Celite, and the filter cake washed with EtOAc (3×150 mL). The filtrate was concentrated under reduced pressure and the residue was purified by column chromatography on silica gel using a gradient of 0-40% EtOAc in hexanes to afford title compound tert-butyl 4-(3-(2,6-bis(benzyloxy)pyridin-3-yl)-1-methyl-1H-indazol-6-yl)piperazine-1-carboxylate (35.6 g, 98% yield) as a solid.

MS (ESI) [M+H]⁺ 607.5.

Step 2: tert-Butyl 4-(3-(2,6-dioxopiperidin-3-yl)-1-methyl-1H-indazol-6-yl)piperazine-1-carboxylate: A mixture of tert-butyl 4-[3-(2,6-dibenzyloxy-3-pyridyl)-1-methyl-indazol-6-yl]piperazine-1-carboxylate (35.6 g, 58.8 mmol) and Pearlman's catalyst (8.90 g, 25 wt. % loading) in EtOH (300 mL) and THE (300 mL) was subjected to hydrogenation (1 atm) at 50° C. for 10 h. The mixture was filtered through Celite, and the filter cake washed with a 1:1 mixture of MeCN and MeOH (4×250 mL). The filtrate was concentrated under reduced pressure and the residue was purified by column chromatography on silica gel using a gradient of 0-100% EtOAc in hexanes to afford title compound tert-butyl 4-(3-(2,6-dioxopiperidin-3-yl)-1-methyl-1H-indazol-6-yl)piperazine-1-carboxylate (21.0 g, 84% yield) as a solid.

MS (ESI) [M+H]⁺ 428.3.

Step 3: 3-(1-Methyl-6-(piperazin-1-yl)-1H-indazol-3-yl)piperidine-2,6-dione (CBM-10): To a solution of tert-butyl 4-[3-(2,6-dioxo-3-piperidyl)-1-methyl-indazol-6-yl]piperazine-1-carboxylate (21.0 g, 49.1 mmol) in 1,4-dioxane (150 mL) was added 4 N HCl in 1,4-dioxane (98.2 mL, 393 mmol) and the reaction mixture was stirred at rt for 20 h. Et₂O (250 mL) was added and the precipitate was collected by filtration, washed with Et₂O (3×30 mL), then dried under vacuum and lyophilized to afford title compound 3-(1-methyl-6-(piperazin-1-yl)-1H-indazol-3-yl)piperidine-2,6-dione (17.6 g, 98% yield) as a solid.

MS (ESI) [M+H]⁺ 328.2.

¹H NMR (500 MHz, DMSO-d₆) δ 10.85 (s, 1H), 9.46 (s, 2H), 7.56 (d, J=8.9 Hz, 1H), 7.04-6.90 (m, 2H), 4.28 (dd, J=9.4, 5.0 Hz, 1H), 3.92 (s, 3H), 3.53-3.41 (m, 4H), 3.23 (s, 4H), 2.73-2.55 (m, 2H), 2.39-2.26 (m, 1H), 2.23-2.08 (m, 1H).

### Example S7. Procedure for CBM-11

### Synthesis of 3-(1-Methyl-7-(piperazin-1-yl)-1H-indazol-3-yl)piperidine-2,6-dione (CBM-11)

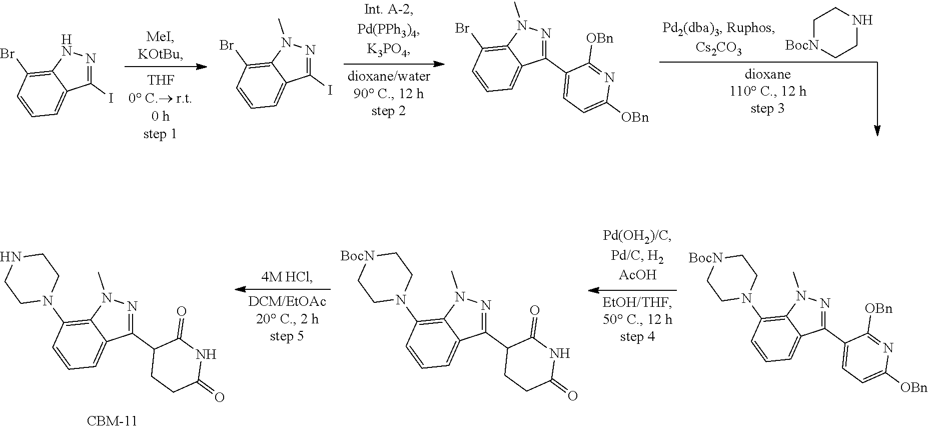

Step 1: 7-Bromo-3-iodo-1-methyl-1H-indazole. Three batches carried out: to a solution of 7-bromo-3-iodo-1H-indazole (480 g, 1.49 mol, 1 equiv) in THE (2.4 L) at 0° C. was added portion-wise t-BuOK (334 g, 2.97 mol, 2 equiv). After the addition, the suspension was stirred at 0° C. for 1 h. Then a solution of CH₃I (422 g, 2.97 mol, 185 mL, 2 equiv) in THE (400 mL) was added dropwise to the cooled (0° C.) reaction mixture. The suspension was then stirred at 25° C. for 3 h. TLC (PE/EtOAc=5/1, Rf=0.5) showed the reaction was completed. The three reaction mixtures were combined, and the resulting suspension was poured into water (10 L) and stirred for 10 min. The aqueous phase was extracted with EtOAc (5.0 L, then 3.0 L). The combined organic phase was washed with brine (3.0 L), dried with anhydrous Na₂SO₄, filtered, and concentrated in vacuo. The crude was purified by column chromatography on silica gel (PE/EtOAc=25/1, 5/1) to give 7-bromo-3-iodo-1-methyl-1H-indazole (900 g, 2.67 mol, 60% yield) as a yellow solid.

¹H NMR (400 MHz, DMSO-d₆) δ ppm 7.72 (d, J=8.4 Hz, 1H), 7.49 (d, J=7.2 Hz, 1H), 7.11 (t, J=7.6 Hz, 1H), 4.34 (s, 3H).

Step 2: 3-(2,6-Bis(benzyloxy)pyridin-3-yl)-7-bromo-1-methyl-1H-indazole. Three batches carried out: to a solution of 7-bromo-3-iodo-1-methyl-1H-indazole (313 g, 929 mmol, 1 equiv) in 1,4-dioxane (2.0 L) and H₂O (1.0 L) was added (2,6-bis(benzyloxy)pyridin-3-yl)boronic acid (389 g, 929 mmol, 80% purity, 1 eq), K₃PO₄ (493 g, 2.32 mol, 2.5 equiv.) and Pd(PPh₃)₄ (21.5 g, 18.6 mmol, 0.02 equiv.). Then the suspension was purged with N₂ three times and stirred at 90° C. for 12 h. The three batches were combined for work up and then the reaction mixture was poured into water (10 L) and stirred for 10 min. The aqueous phase was extracted with EtOAc (5 L, then 3 L). The combined organic phase was washed with brine (3 L), dried with anhydrous Na₂SO₄, filtered, and concentrated in vacuo. The crude was purified by column chromatography on silica gel (PE/EtOAc=25/1, 5/1). The residue was triturated with PE/EtOAc (2/1) at 25° C. for 3 h, then the solids were collected by vacuum filtration to give 3-(2,6-bis(benzyloxy)pyridin-3-yl)-7-bromo-1-methyl-1H-indazole (920 g, 63% yield) as an off-white solid.

¹H NMR (400 MHz, DMSO-d₆) δ ppm 7.86 (d, J=8.4 Hz, 1H), 7.54 (dd, J=8.0, 0.8 Hz, 1H), 7.37 (dd, J=7.2, 0.8 Hz, 1H), 7.33 (m, 2H), 7.28 (m, 8H), 6.94 (t, J=7.6 Hz, 1H), 6.60 (d, J=7.6 Hz, 1H), 5.43 (s, 4H), 4.36 (s, 3H).

Step 3: tert-Butyl 4-(3-(2,6-bis(benzyloxy)pyridin-3-yl)-1-methyl-1H-indazol-7-yl)piperazine-1-carboxylate. To a solution of 3-(2,6-bis(benzyloxy)pyridin-3-yl)-7-bromo-1-methyl-1H-indazole (100 g, 200 mmol,) and tert-butyl piperazine-1-carboxylate (55.8 g, 300 mmol) in 1,4-dioxane (700 mL) was added Cs₂CO₃ (130 g, 400 mmol), RuPhos (18.6 g, 40 mmol) and Pd₂(dba)₃ (18.3 g, 20 mmol), then the suspension was purged with N₂ three times and stirred at 110° C. for 12 h. TLC (PE/EtOAc=3/1, R_{f}=0.6) showed the reaction was completed. The reaction was cooled to 20° C. and filtered through a pad of Celite. The filtrate was concentrated under vacuum and the residue was purified by silica gel chromatography (100-200 mesh silica gel, PE/EtOAc=20/1, 3/1) to give the product. The product was further purified by trituration with PE/EtOAc=(2/1, 200 mL) for 1 h. The solid was collected by filtration and dried under vacuum to affordtert-Butyl 4-(3-(2,6-bis(benzyloxy)pyridin-3-yl)-1-methyl-1H-indazol-7-yl)piperazine-1-carboxylate (84 g, 133 mmol, 67% yield, 96% purity) as a yellow solid.

¹H NMR (400 MHz DMSO-d₆) δ ppm 7.85 (d, J=8.4 Hz, 1H), 7.27-7.45 (m, 12H), 6.96-6.99 (m, 2H), 6.53 (d, J=8.0 Hz, 1H), 5.47 (s, 2H), 5.40 (s, 2H), 4.41 (s, 3H), 4.10-4.16 (m, 2H), 3.20-3.23 (m, 4H), 2.84-2.89 (m, 2H), 1.51 (s, 9H).

Step 4: tert-Butyl 4-(3-(2,6-dioxopiperidin-3-yl)-1-methyl-1H-indazol-7-yl)piperazine-1-carboxylate. To a suspension of tert-butyl 4-(3-(2,6-bis(benzyloxy)pyridin-3-yl)-1-methyl-1H-indazol-7-yl)piperazine-1-carboxylate (42 g, 69.3 mmol) and AcOH (4.16 g, 69.3 mmol, 3.97 mL) in THE (210 mL) and EtOH (210 mL) was added 10% Pd/C (8.0 g) and 20% Pd(OH)₂ (8.0 g, 57 mmol), then the black suspension was purged with H₂ three times and stirred at 50° C. under 50 psi for 12 h. The suspension was filtered through a pad of celite, and the filter cake was washed with hot THE (2 L). The filtrate was concentrated under vacuum at 45° C. to get the crude product. The crude material was purified by silica gel chromatography (100-200 mesh silica gel, DCM/MeOH=0/1, 10/1) to give a solid. The solid was further triturated with MTBE (50 mL) for 1 h. The solid was collected by filtration and dried under vacuum. tert-butyl 4-(3-(2,6-dioxopiperidin-3-yl)-1-methyl-1H-indazol-7-yl)piperazine-1-carboxylate (18.6 g, 41.7 mmol, 30% yield, 96% purity) was obtained as a blue solid.

MS (ESI) [M+H]⁺ 428.4.

Step 5: 3-(1-Methyl-7-(piperazin-1-yl)-1H-indazol-3-yl)piperidine-2,6-dione (CBM-11). To a solution of tert-butyl 4-(3-(2,6-dioxopiperidin-3-yl)-1-methyl-1H-indazol-7-yl)piperazine-1-carboxylate (18.6 g, 43.5 mmol, 1 eq) in DCM (420 mL) was added HCl/EtOAc (4 M, 93.0 mL), then the suspension was stirred at 20° C. for 2 h. The solid was collected by filtration and dried under vacuum at 45° C. for 2 h. The solid was suspended in MeCN (100 mL) and dried under vacuum at 45° C. for 2 h. The operation was repeated two more times. 3-(1-methyl-7-(piperazin-1-yl)-1H-indazol-3-yl)piperidine-2,6-dione (16.5 g, 41.2 mmol, 95% yield, 2HCl) was obtained as a light blue solid.

¹H NMR (400 MHz, DMSO-d₆) δ ppm 10.87 (s, 1H), 9.49-9.58 (m, 2H), 7.45 (d, J=6.8 Hz, 1H), 7.03-7.06 (m, 2H), 4.33-4.37 (m, 1H), 4.24 (s, 3H), 3.15-3.44 (m, 8H), 2.60-2.67 (m, 2H), 2.31-2.50 (m, 1H), 2.14-2.18 (m, 1H).

### Example S8. Procedure for CBM-19

### Synthesis of 1-Methyl-3-(4-(piperazin-1-yl)phenyl)piperidine-2,6-dione (CBM-19)

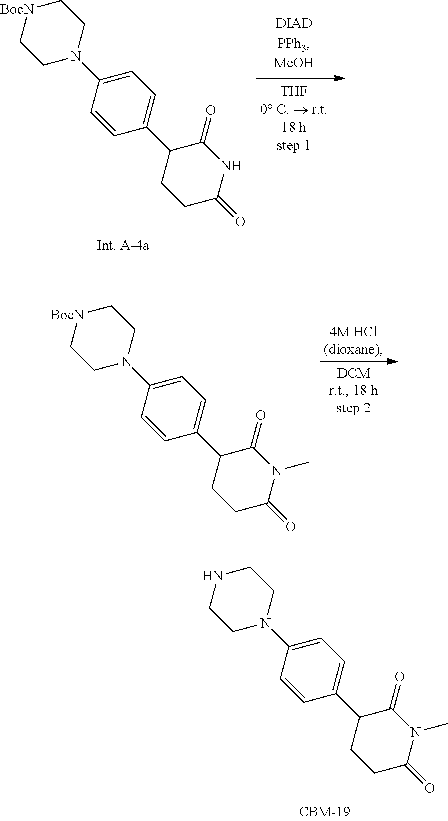 [SMILES: CC(C)(C)OC(=O)N1CCN(c2ccc(C3CCC(=O)NC3=O)cc2)CC1 ; CN1C(=O)CCC(c2ccc(N3CCN(C(=O)OC(C)(C)C)CC3)cc2)C1=O ; CN1C(=O)CCC(c2ccc(N3CCNCC3)cc2)C1=O]

Step 1: tert-Butyl 4-(4-(1-methyl-2,6-dioxopiperidin-3-yl)phenyl)piperazine-1-carboxylate: To a solution of MeOH (0.02 mL, 0.57 mmol, 1.0 equiv.) in THF (6.0 mL) was added DIAD (0.11 mL, 0.57 mmol, 1.0 equiv.) followed by tert-butyl 4-[4-(2,6-dioxo-3-piperidyl)phenyl]piperazine-1-carboxylate Int. A-4a (300 mg, 0.57 mmol, 1.0 equiv.) at 0° C. Then, a solution of PPh₃ (224.4 mg, 0.86 mmol, 1.5 equiv.) in THE (3.62 mL; for a total concentration of 0.06 M) was added dropwise and the mixture warmed up to r.t. and stirred 18 h. The solvent was removed under reduced pressure and the crude residue was purified by reverse phase column chromatography (5% to 70% MeCN in water w//0.1% formic acid). The fractions were concentrated to a minimum volume of water, neutralized with NaHCO₃, and extracted with DCM (3×). The combined organic layers were dried over MgSO₄, filtered, and concentrated to yield the tert-butyl 4-(4-(1-methyl-2,6-dioxopiperidin-3-yl)phenyl)piperazine-1-carboxylate (156 mg, 84%) as an off-white solid.

LCMS: [M+H]⁺=388.2.

¹H NMR (400 MHz, DMSO-d₆): δ ppm 1.42 (s, 9H), 1.95-2.05 (m, 1H), 2.07-2.19 (m, 1H), 2.56-2.66 (m, 1H), 2.69-2.81 (m, 1H), 3.02 (s, 3H), 3.04-3.09 (m, 4H), 3.41-3.48 (m, 4H), 3.84 (dd, J=11.0, 4.9 Hz, 1H), 6.91 (d, J=8.6 Hz, 2H), 7.06 (d, J=8.6 Hz, 2H).

Step 2: 1-Methyl-3-(4-(piperazin-1-yl)phenyl)piperidine-2,6-dione (CBM-19): To a solution of tert-butyl 4-[4-(1-methyl-2,6-dioxo-3-piperidyl)phenyl]piperazine-1-carboxylate (184 mg, 0.47 mmol) in DCM (4.74 mL, 0.10 M) was added 4 M HCl in dioxane (1.78 mL, 7.12 mmol, 15.0 equiv.). The reaction was stirred at r.t. for 18 h. The solvent was removed under reduced pressure to yield the HCl salt of 1-methyl-3-(4-(piperazin-1-yl)phenyl)piperidine-2,6-dione (CBM-19) (153 mg, 99.9% yield) as a white solid.

LCMS: [M+H]⁺=288.2.

¹H NMR (400 MHz, DMSO-d₆): δ ppm 1.95-2.04 (m, 1H), 2.09-2.20 (m, 1H), 2.64 (s, 1H), 2.70-2.82 (m, 1H), 3.02 (s, 3H), 3.18-3.26 (m, 4H), 3.30-3.37 (m, 4H), 3.82-3.91 (m, 1H), 6.95 (d, J=8.6 Hz, 2H), 7.10 (d, J=8.6 Hz, 2H), 8.89 (br s, 1H).

### Example S9. Procedure for CBM-25

### Synthesis of 3-(4-(2,6-Diazaspiro[3.3]heptan-2-yl)phenyl)piperidine-2,6-dione (CBM-25)

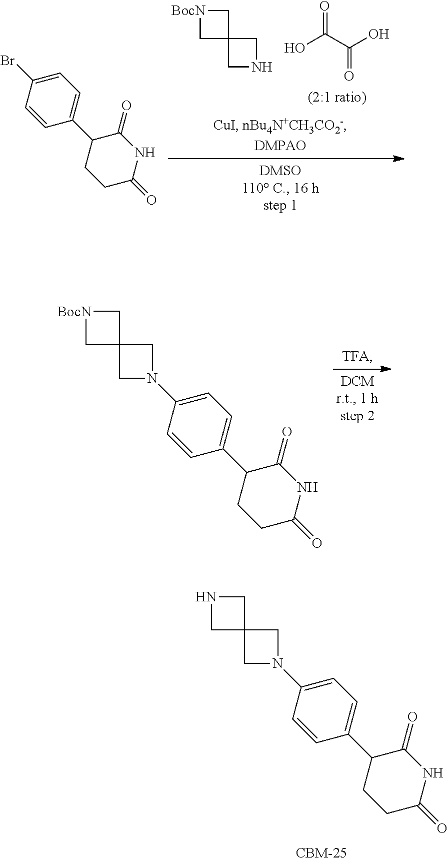

Step 1: tert-Butyl 6-(4-(2,6-dioxopiperidin-3-yl)phenyl)-2,6-diazaspiro[3.3]heptane-2-carboxylate: To a screw-top 250 mL flask was added DMSO (34.7 mL) and molecular sieves (~10 beads, 3A). The solvent was stirred at room temperature with nitrogen bubbling through it for 10 minutes. Then, 3-(4-bromophenyl)piperidine-2,6-dione (1.86 g, 6.94 mmol), tert-butyl 2,6-diazaspiro[3.3]heptane-2-carboxylate oxalic acid salt (2.03 g, 4.16 mmol), DMPAO (1.34 g, 6.94 mmol), CuI (660.62 mg, 3.47 mmol) and tetrabutylammonium acetate (6.28 g, 20.81 mmol) were added and nitrogen was bubbled through the solution for 10 minutes. The reaction mixture was stirred at 110° C. until completion (ca. 16 h). Water was added and the aqueous phase was extracted thrice with EtOAc. The organic phases were combined, washed with water and brine, dried over Na₂SO₄ and concentrated to dryness. The resulting residue was first purified by reverse phase column chromatography (5 to 100% MeCN in water w/0.1% formic acid), then by silica gel column chromatography (0 to 100% EtOAc in heptane), affording tert-butyl 6-(4-(2,6-dioxopiperidin-3-yl)phenyl)-2,6-diazaspiro[3.3]heptane-2-carboxylate (136 mg, 0.35 mmol, 5% yield) as an off-white solid.

LCMS: [M+H]⁺=386.2.

¹H NMR (400 MHz, DMSO-d₆): δ ppm 1.38 (s, 9H), 1.93-2.02 (m, 1H), 2.07 (s, 2H), 2.56-2.65 (m, 1H), 3.69 (dd, J=10.8, 4.9 Hz, 1H), 3.98-4.04 (m, 5H), 6.38 (d, J=8.6 Hz, 2H), 7.00 (d, J=8.3 Hz, 2H), 10.75 (s, 1H).

Step 2: 3-(4-(2,6-Diazaspiro[3.3]heptan-2-yl)phenyl)piperidine-2,6-dione (CBM-25): In a round-bottom flask, tert-butyl 6-[4-(2,6-dioxo-3-piperidyl)phenyl]-2,6-diazaspiro[3.3]heptane-2-carboxylate 3 (152.8 mg, 0.40 mmol) was dissolved in DCM (2 mL), then trifluoroacetic acid (0.61 mL, 7.93 mmol) was added and the mixture was stirred at room temperature for 30 min. The solvent was evaporated under reduced pressure and co-evaporated thrice with acetonitrile to afford 3-(4-(2,6-diazaspiro[3.3]heptan-2-yl)phenyl)piperidine-2,6-dione (CBM-25) (204.1 mg, quant. yield, TFA salt) as a grey oil which was used directly in the next step without further purification.

¹H NMR (400 MHz, DMSO-d₆): δ ppm 1.93-2.01 (m, 1H), 2.08-2.14 (m, 1H), 2.57-2.65 (m, 1H), 3.70 (dd, J=11.0, 4.9 Hz, 1H), 4.13-4.17 (m, 4H), 6.42 (d, J=8.6 Hz, 2 H), 7.02 (d, J=8.6 Hz, 2H), 8.43 (br s, 2H), 10.76 (s, 1H).

### Example S10. Procedure for CBM-26

### Synthesis of 1-(4-(Piperazin-1-yl)phenyl)pyrimidine-2,4(1H,3H)-dione (CBM-26)

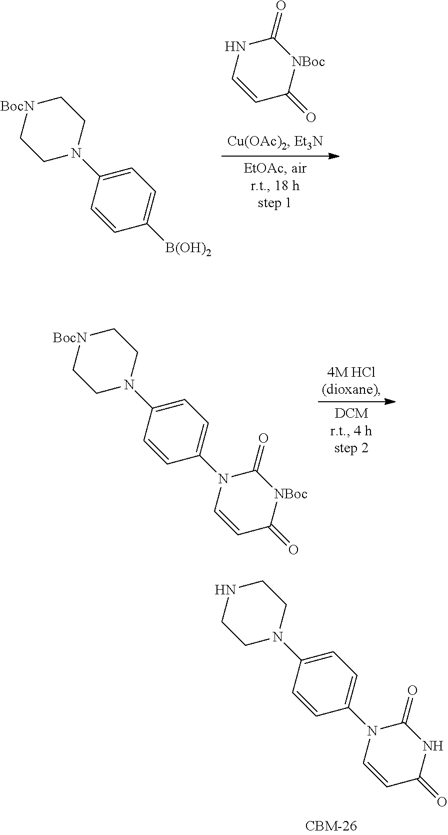

Step 1: tert-Butyl 3-(4-(4-(tert-butoxycarbonyl)piperazin-1-yl)phenyl)-2,6-dioxo-3,6-dihydropyrimidine-1(2H)-carboxylate: To a solution of tert-butyl 2,4-dioxo-1H-pyrimidine-3-carboxylate (300 mg, 1.41 mmol, 1.0 equiv.) in EtOAc (10 mL) was added [4-(4-tert-butoxycarbonylpiperazin-1-yl)phenyl]boronic acid (562 mg, 1.84 mmol, 1.3 equiv.), Et₃N (0.49 mL, 3.53 mmol, 2.5 equiv.) followed by Cu(OAc)₂ (385 mg, 2.12 mmol, 1.5 equiv.). The reaction was stirred under air at room temperature overnight. The reaction mixture was diluted with EtOAc and water. The phases were separated, and the organic layer was washed with NH₄Cl (2×), water, brine, dried over sodium sulfate, filtered, and concentrated. The mixture was purified by reverse phase column chromatography (5% to 100% MeCN water w/ 0.1% formic acid) to give tert-butyl 3-[4-(4-tert-butoxycarbonylpiperazin-1-yl)phenyl]-2,6-dioxo-pyrimidine-1-carboxylate (260 mg, 39% yield) as an off-white solid.

LCMS: [M+H]⁺=473.2.

¹H NMR (400 MHz, DMSO-d₆): δ ppm 1.42 (s, 9H), 1.51 (s, 9H), 3.11-3.20 (m, 4H), 3.41-3.51 (m, 4H), 5.81 (d, J=7.8 Hz, 1H), 6.99-7.07 (m, 2H), 7.26-7.34 (m, 2H), 7.74-7.80 (m, 1H).

Step 2: 1-(4-(Piperazin-1-yl)phenyl)pyrimidine-2,4(1H,3H)-dine (CBM-26): To a solution of tert-butyl 3-[4-(4-tert-butoxycarbonylpiperazin-1-yl)phenyl]-2,6-dioxo-pyrimidine-1-carboxylate (260 mg, 0.550 mmol, 1.0 equiv.) in CH₂Cl₂ (2 mL) was added HCl (1 mL, 5.5 mmol, 10 equiv.) at room temperature for 4 h. The solvent was evaporated, co-evaporated with MeCN (3×) and MTBE (2×) to give 1-(4-piperazin-1-ylphenyl)pyrimidine-2,4-dione (CBM-26) (240 mg, quant., bis-HCl salt) as an off-white solid.

LCMS: [M+H]⁺=473.2.

¹H NMR (400 MHz, DMSO-d₆): δ 3.22 (br s, 4H), 3.38-3.47 (m, 4H), 5.63 (dd, J=7.8, 2.2 Hz, 1H), 7.06 (d, J=9.0 Hz, 2H), 7.28 (d, J=9.0 Hz, 2H), 7.63 (d, J=7.8 Hz, 1H), 9.17 (br s, 2H), 11.38 (s, 1H).

### Example S11. Procedure for CBM-30

### Synthesis of 5-Fluoro-1-(4-(piperazin-1-yl)phenyl)pyrimidine-2,4(1H,3H)-dione (CBM-30)

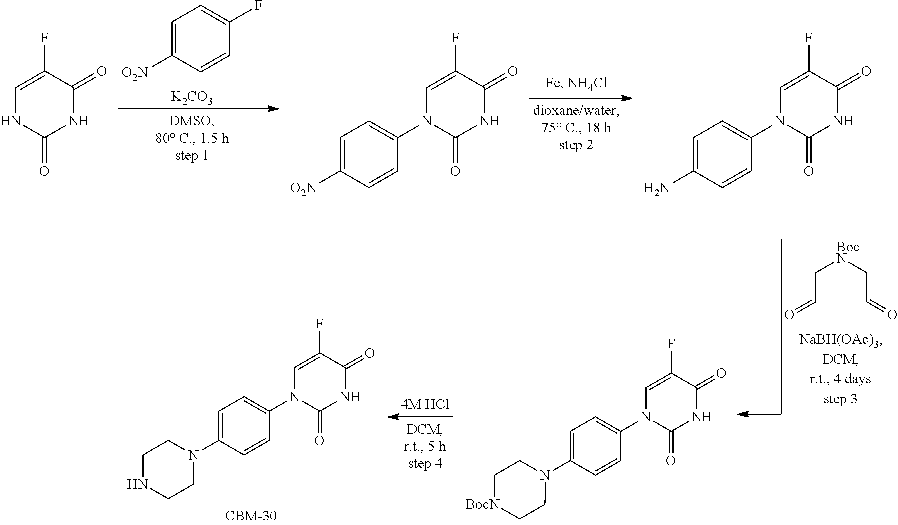

Step 1: 5-Fluoro-1-(4-nitrophenyl)pyrimidine-2,4(1H,3H)-dione: To a solution of 5-fluoro-1H-pyrimidine-2,4-dione (500 mg, 3.84 mmol, 1.0 equiv.) in DMSO (10 mL) was added 1-fluoro-4-nitro-benzene (542 mg, 3.84 mmol, 1.0 equiv.) and K₂CO₃ (1.33 g, 9.61 mmol, 2.5 equiv.) at room temperature. Then, the reaction mixture was heated to 80° C. for 1.5 h. LCMS showed 55-60% conversion. The reaction mixture was cooled to room temperature and water (5 mL) was added. The mixture was diluted with water and EtOAc. The phases were separated, and the water of the aqueous phase was concentrated and purified by reverse phase column chromatography (5% to 50% MeCN in water w/ 0.1% formic acid), affording 5-fluoro-1-(4-nitrophenyl)pyrimidine-2,4(1H,3H)-dione (291 mg, 30%) as a white solid.

LCMS: [M+H]⁺=252.2.

¹H NMR (400 MHz, DMSO-d₆): δ ppm 7.72-7.82 (m, 2H), 8.29-8.41 (m, 3H), 12.04-12.15 (m, 1H).

Step 2: 1-(4-Aminophenyl)-5-fluoropyrimidine-2,4(1H,3H)-dione: To a solution of 5-fluoro-1-(4-nitrophenyl)pyrimidine-2,4(1H,3H)-dione (360 mg, 1.43 mmol, 1.0 equiv.) in 1,4-dioxane (3 mL) and water (3 mL) was added Fe (640 mg, 11.47 mmol, 8.0 equiv.) and NH₄Cl (613 mg, 11.47 mmol, 8.0 equiv.) at room temperature. The reaction mixture was heated at 75° C., overnight. LCMS showed full conversion. The mixture was filtered over celite and washed with MeCN to afford 1-(4-aminophenyl)-5-fluoropyrimidine-2,4(1H,3H)-dione (260 mg, 82%) as an off-white solid.

LCMS: [M+H]⁺=222.2.

¹H NMR (400 MHz, DMSO-d₆): δ ppm 5.35 (s, 2H), 6.52-6.64 (m, 2H), 6.98-7.06 (m, 2H), 7.16-7.27 (m, 1H), 8.05 (d, J=6.6 Hz, 1H).

¹⁹F NMR (377 MHz, DMSO-d₆): δ ppm −170.47 (d, J=6.8 Hz, 1 F).

Step 3: tert-Butyl 4-(4-(5-fluoro-2,4-dioxo-3,4-dihydropyrimidin-1(2H)-yl)phenyl)piperazine-1-carboxylate: To a solution of 1-(4-aminophenyl)-5-fluoropyrimidine-2,4(1H,3H)-dione (150 mg, 0.68 mmol, 1.0 equiv.) and tert-butyl N,N-bis(2-oxoethyl)carbamate (205 mg, 1.02 mmol, 1.5 equiv.) in DCE (5 mL) was added NaBH(OAc)₃ (287 mg, 1.36 mmol, 2.0 equiv.) at room temperature. The reaction mixture was stirred at room temperature overnight. LCMS showed enamine intermediate with traces of desired product. NaBH(OAc)₃ (287 mg, 1.36 mmol, 2.0 equiv.) was added and the mixture was stirred at room temperature overnight. LCMS showed around 50% conversion. NaBH(OAc)₃ (287 mg, 1.36 mmol, 2.0 equiv.) was added and the mixture was stirred at room temperature for 72 h. LCMS showed almost full conversion. Water and CH₂Cl₂ were added, and the phases were separated. The aqueous phase was extracted with CH₂Cl₂ (2×), the combined organic layer was dried over sodium sulfate, filtered, and concentrated. The crude product was purified by reserve phase column chromatography (5 to 100% MeCN in water w/ 0.1% FA) to give tert-butyl 4-(4-(5-fluoro-2,4-dioxo-3,4-dihydropyrimidin-1(2H)-yl)phenyl)piperazine-1-carboxylate (60 mg, 23% yield) as an off-white solid.

LCMS: [M+H]⁺=391.2.

¹H NMR (400 MHz, DMSO-d₆): δ ppm 1.42 (s, 9H), 3.11-3.21 (m, 4H), 3.42-3.50 (m, 4H), 6.98-7.04 (m, 2H), 7.23-7.31 (m, 2H), 8.12 (d, J=6.6 Hz, 1H), 11.85 (br s, 1H).

¹⁹F NMR (377 MHz, DMSO-d₆): δ ppm −170.13 (br d, J=5.4 Hz, 1 F).

Step 4: 5-Fluoro-1-(4-(piperazin-1-yl)phenyl)pyrimidine-2,4(1H,3H)-dione (CBM-30): To a solution of tert-butyl 4-(4-(5-fluoro-2,4-dioxo-3,4-dihydropyrimidin-1(2H)-yl)phenyl)piperazine-1-carboxylate (60 mg, 0.15 mmol, 1.0 equiv.) in CH₂Cl₂ (2 mL) was added HCl (1 mL, 3.75 mmol, 25 equiv.) at room temperature and the reaction mixture was stirred at room temperature for 5 h. LCMS showed full conversion. The solvent was evaporated, co-evaporated with MeCN (3×) and CH₂Cl₂ (2×) to afford 5-fluoro-1-(4-(piperazin-1-yl)phenyl)pyrimidine-2,4(1H,3H)-dione (CBM-30) (60 mg, quant. yield, bis-HCl salt) as an off-white solid.

LCMS: [M+H]⁺=291.2.

¹H NMR (400 MHz, DMSO-d₆): δ ppm 3.19-3.27 (m, 4H), 3.38-3.45 (m, 4H), 7.02-7.10 (m, 2H), 7.26-7.35 (m, 2H), 8.12 (d, J=6.6 Hz, 1H), 8.93 (br dd, J=2.2, 1.5 Hz, 2H), 11.88 (d, J=5.1 Hz, 1H).

¹⁹F NMR (377 MHz, DMSO-d₆): δ ppm −170.14-−169.97 (m, 1 F).

### Example S12. Procedure for CBM-31

### Synthesis of 1-(1-(Piperidin-4-yl)-1H-pyrazol-4-yl)dihydropyrimidine-2,4(1H,3H)-dione (CBM-31)

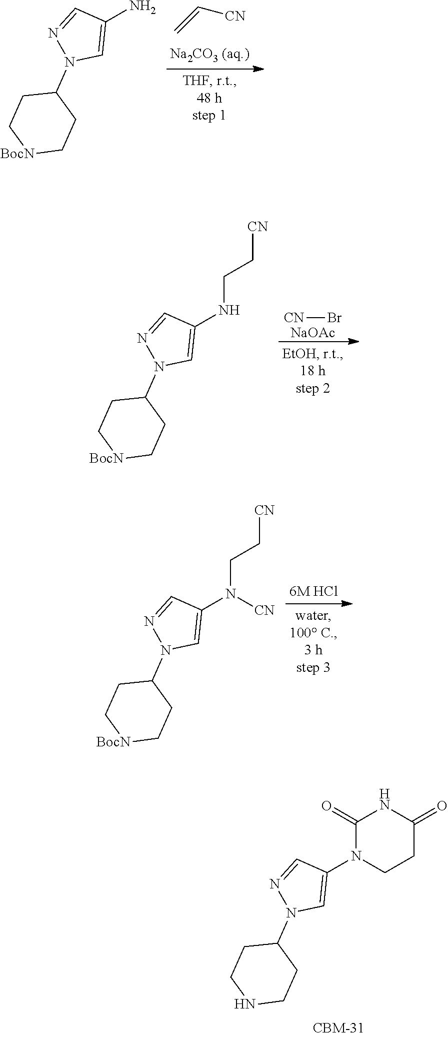

Step 1: tert-Butyl 4-(4-((2-cyanoethyl)amino)-1H-pyrazol-1-yl)piperidine-1-carboxylate: A solution of tert-butyl 4-(4-aminopyrazol-1-yl)piperidine-1-carboxylate (1.37 g, 5.14 mmol), aq. sat. Na₂CO₃ solution (57 mL, 5.71 mmol) and prop-2-enenitrile (6.85 mL, 104.57 mmol) in THF (10 mL) was stirred at room temperature for 48 h. The aqueous solution was extracted with EtOAc, and the combined organic phase was washed with NaCl, dried over MgSO₄. The mixture was concentrated to dryness and purified by silica gel column chromatography (0% to 100% EtOAc in heptane), affording tert-butyl 4-(4-((2-cyanoethyl)amino)-1H-pyrazol-1-yl)piperidine-1-carboxylate (1.6 g, 97% yield) as a brown oil.

LCMS: [M+H]⁺=320.2.

¹H NMR (400 MHz, CDCl₃): δ ppm 1.41 (s, 9H), 1.71 (ddd, J=24.0, 12.0, 4.2 Hz, 2H), 1.88-1.95 (m, 2H), 2.65 (t, J=6.6 Hz, 2H), 2.87 (br s, 2H), 3.10 (q, J=6.6 Hz, 2H), 4.00 (br s, 2H), 4.09-4.22 (m, 1H), 4.66 (t, J=6.4 Hz, 1H), 7.00 (br s, 1H), 7.25 (br s, 1H).

Step 2: tert-Butyl 4-(4-(N-(2-cyanoethyl)cyanamido)-1H-pyrazol-1-yl)piperidine-1-carboxylate: A solution of tert-butyl 4-(4-((2-cyanoethyl)amino)-1H-pyrazol-1-yl)piperidine-1-carboxylate (1.6 g, 5.01 mmol) in EtOH (20 mL) was added slowly to a solution at 0° C. of carbononitridic bromide (6.68 mL, 20.04 mmol) and NaOAc (1.42 mL, 12.52 mmol) in EtOH (5 mL). The reaction was stirred until completion by LCMS (ca. 18 h). The mixture was concentrated to dryness and the residue was washed with aq. solution of citric acid 5%, extracted with EtOAc. The organic phase was washed with brine and dried over MgSO₄ and the crude was purified by silica gel column chromatography (0% to 100% EtOAc in heptane) affording tert-Butyl 4-(4-(N-(2-cyanoethyl)cyanamido)-1H-pyrazol-1-yl)piperidine-1-carboxylate (1.5 g, 81% yield) as an orange oil.

LCMS: [M+Na]⁺=367.0.

¹H NMR (400 MHz, CDCl₃): δ ppm 1.41 (s, 9H), 1.74 (ddd, J=24.9, 12.2, 4.4 Hz, 2H), 1.91-1.98 (m, 2H), 2.79-2.99 (m, J=6.4, 6.4 Hz, 4H), 3.77 (t, J=6.5 Hz, 2H), 3.96-4.02 (m, 2H), 4.25-4.36 (m, 1H), 7.49 (d, J=0.7 Hz, 1H), 7.97 (d, J=0.7 Hz, 1H).

Step 3: 1-(1-(Piperidin-4-yl)-1H-pyrazol-4-yl)dihydropyrimidine-2,4(1H,3H)-dione (CBM-31): A solution of tert-butyl 4-(4-(N-(2-cyanoethyl)cyanamido)-1H-pyrazol-1-yl)piperidine-1-carboxylate (1.5 g, 4.36 mmol) in aq. solution of 6 N HCl (4.5 mL, 27 mmol) was heated to 100° C. until complete by LCMS (ca. 3 h). The reaction mixture was concentrated to dryness and the crude material suspended in a mixture of 30% MeOH/DCM, neutralized with sat. sol. of NaHCO₃ (pH ~7). The aqueous phase was concentrated to dryness and the residue was purified by reverse phase column chromatography (0% to 100% MeCN in water w/ 0.1% formic acid) affording 1-(1-(piperidin-4-yl)-1H-pyrazol-4-yl)dihydropyrimidine-2,4(1H,3H)-dione (CBM-31) after lyophilization (764.7 mg, 67% yield) as a tan solid.

LCMS: [M+H]⁺=264.2.

¹H NMR (400 MHz, DMSO-d₆): δ ppm 1.74 (ddd, J=24.0, 11.7, 4.2 Hz, 2H), 1.85-1.94 (m, 2H), 2.53-2.61 (m, 2H), 2.68 (t, J=6.8 Hz, 2H), 2.97-3.06 (m, J=12.5 Hz, 2H), 3.75 (t, J=6.8 Hz, 2H), 4.04-4.20 (m, 1H), 7.59 (s, 1H), 7.92 (s, 1H), 10.36 (br s, 1H).

### Procedures for Target-binding Moieties (TBMs)

### Example S13. General Procedure for TBM-D

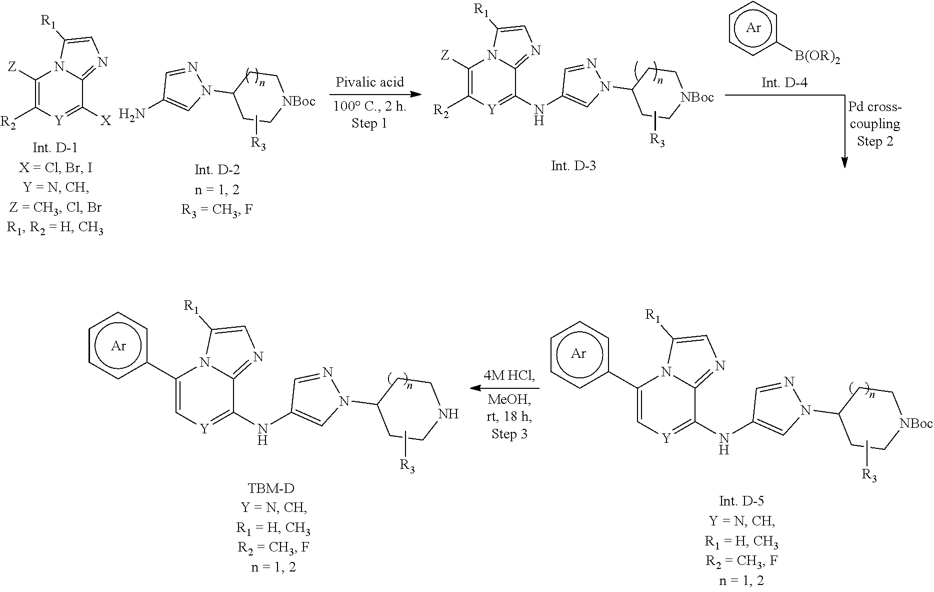

### Synthesis of TBM-D: Step 1

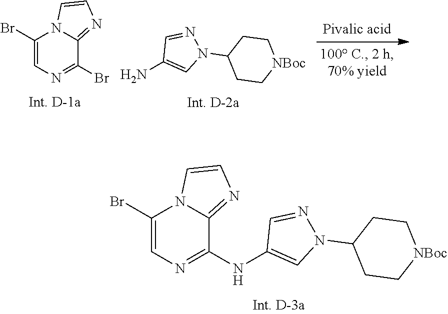 [SMILES: Brc1ncc(Br)n2ccnc12 ; CC(C)(C)OC(=O)N1CCC(n2cc(N)cn2)CC1 ; CC(C)(C)OC(=O)N1CCC(n2cc(Nc3ncc(Br)n4ccnc34)cn2)CC1]

To a round-bottom flask was added 5,8-dibromoimidazo[1,2-a]pyrazine Int. D-la (5.0 g, 18 mmol, 1 equiv.), tert-butyl 4-(4-aminopyrazol-1-yl)piperidine-1-carboxylate Int. D-2a (5.29 g, 19.8 mmol, 1.1 equiv.) and pivalic acid (27.66 g, 271 mmol, 15 equiv.). Then, the flask was put in a pre-heated oil bath at 100° C. After 2 h, LCMS showed full conversion with traces of deprotected product. The brown residue was quenched with a saturated solution of NaHCO₃ (until pH 7-9) and extracted with MeTHF (3×). The combined organic layers were washed with brine, dried over Na₂SO₄ and concentrated to give a dark red residue. The residue was then purified by reverse phase column chromatography (5% to 100% MeOH in water w/ 0.1% formic acid). The fractions were combined and concentrated to give tert-butyl 4-(4-((5-bromoimidazo[1,2-a]pyrazin-8-yl)amino)-1H-pyrazol-1-yl)piperidine-1-carboxylate Int. D-3a (5.99 g, 70% yield) as a brown solid.

LCMS: [M+H]⁺=462.2, 464.2.

¹H NMR (400 MHz, DMSO-d₆) δ ppm 1.42 (s, 9H), 1.76 (qd, J=12.1, 4.2 Hz, 2H), 1.99 (br dd, J=12.3, 2.3 Hz, 2H), 2.78-2.99 (m, 2H), 3.97-4.12 (m, 2H), 4.30-4.39 (m, 1H), 7.59 (s, 1H), 7.70 (d, J=1.2 Hz, 1H), 7.76 (s, 1H), 7.98 (d, J=1.0 Hz, 1H), 8.18 (s, 1H), 10.00 (s, 1H).

TABLE 16
List of intermediates used in TBM-D: Step 1
Starting Materials

| Int. D | Structure | Source |
|---|---|---|
| Int. D-1a | 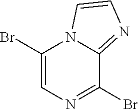 [SMILES: Brc1ncc(Br)n2ccnc12] | Commercially available |
| Int. D-1b | 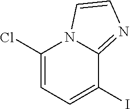 [SMILES: Clc1ccc(I)c2nccn12] | Commercially available |
| Int. D-1c | 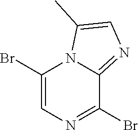 | Synthesis provided below |
| Int. D-1d | 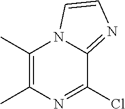 | Synthesis provided below |
| Int. D-2a | 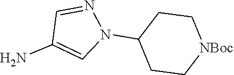 [SMILES: CC(C)(C)OC(=O)N1CCC(n2cc(N)cn2)CC1] | Commercially available |
| Int. D-2b | 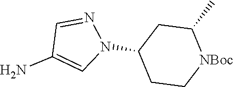 [SMILES: C[C@H]1C[C@@H](n2cc(N)cn2)CCN1C(=O)OC(C)(C)C] | Synthesis provided below |
| Int. D-2c | 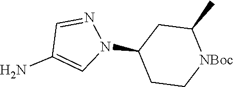 [SMILES: C[C@@H]1C[C@H](n2cc(N)cn2)CCN1C(=O)OC(C)(C)C] | Synthesis provided below |
| Int. D-2d | 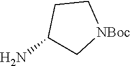 [SMILES: CC(C)(C)OC(=O)N1CC[C@@H](N)C1] | Commercially available |
| Int. D-2e | 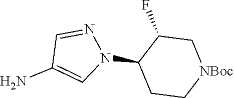 [SMILES: CC(C)(C)OC(=O)N1CC[C@@H](n2cc(N)cn2)[C@H](F)C1] | Synthesis provided below |
| Int. D-2f | 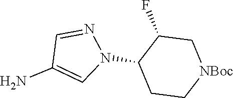 [SMILES: CC(C)(C)OC(=O)N1CC[C@H](n2cc(N)cn2)[C@H](F)C1] | Synthesis provided below |
| Int. D-2g | 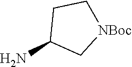 [SMILES: CC(C)(C)OC(=O)N1CC[C@H](N)C1] | Commercially available |
| Int. D-2h | 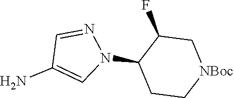 [SMILES: CC(C)(C)OC(=O)N1CC[C@@H](n2cc(N)cn2)[C@@H](F)C1] | Synthesis provided below |
| Int. D-2i | 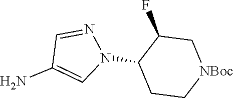 [SMILES: CC(C)(C)OC(=O)N1CC[C@H](n2cc(N)cn2)[C@@H](F)C1] | Synthesis provided below |
| Int. D-2j | 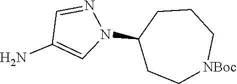 [SMILES: CC(C)(C)OC(=O)N1CCC[C@H](n2cc(N)cn2)CC1] | Synthesis provided below |
| Int. D-2k | 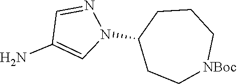 [SMILES: CC(C)(C)OC(=O)N1CCC[C@@H](n2cc(N)cn2)CC1] | Synthesis provided below |

### Synthesis of 5,8-dibromo-3-methylimidazo[1,2-a]pyrazine (Int. D-1c)

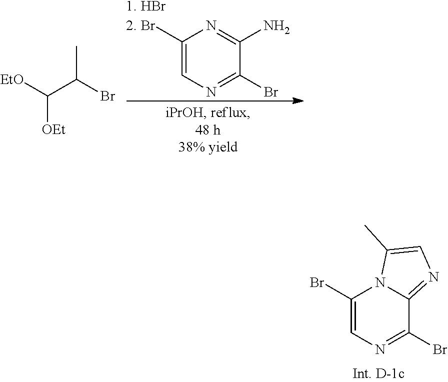

To a round-bottom flask was added 2-bromo-1,1-diethoxy-propane 1 (1.59 mL, 9.9 mmol, 5 equiv.) in IPA (9.9 mL, 0.2 M). To the reaction mixture was added HBr (1.18 mL, 21.7 mmol, 11 equiv.) and the reaction mixture was heated to 95° C. and stirred overnight. The resulting yellow solution was cooled to room temperature, neutralized with solid NaHCO₃, filtered, and rinsed with isopropanol. The obtained solution was put in a flask and 3,6-dibromopyrazin-2-amine (500 mg, 2.0 mmol) was added and heated to reflux at 95° C. and reacted overnight. The solution was cooled down to room temperature and concentrated. The residue was dissolved in EtOAc, washed with NaHCO₃ (2×) and brine, dried over sodium sulfate, filtered, and concentrated. The crude product was purified by reverse phase column chromatography (5% to 100% in water w/ 0.1% formic acid). The fractions were combined and concentrated to afford 5,8-dibromo-3-methylimidazo[1,2-a]pyrazine Int. D-lc (212 mg, 36% yield) as a yellow solid.

LCMS: [M+H]⁺=292.0.

¹H NMR (400 MHz, DMSO-d₆) δ ppm 2.82 (s, 3H), 7.72 (s, 1H), 7.84 (s, 1H).

### Synthesis of 8-chloro-5,6-dimethylimidazo[1,2-a]pyrazine (Int. D-1d)

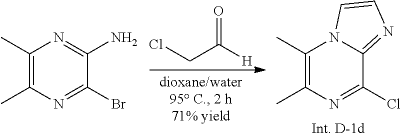

A pressure tube was charged with 3-bromo-5,6-dimethyl-pyrazin-2-amine (200 mg, 0.99 mmol), 2-chloroacetaldehyde in H₂O (50% w/w, 1.34 mL, 9.9 mmol) and 1,4-dioxane (0.825 mL), purged with N₂, sealed, and heated to 90° C. until complete by LCMS analysis (ca. 2 h). The reaction mixture was concentrated to dryness and the crude material was taken up in DMSO/1 M aq. NaOH and purified by reverse phase column chromatography (5% MeCN in water w/ pH 10 buffer, then 5 to 100% MeCN in water w/ formic acid) to afford 8-chloro-5,6-dimethylimidazo[1,2-a]pyrazine Int. D-1d (128 mg, 71% yield) as an off-white solid.

LCMS: [M+H]⁺=182.2.

¹H NMR (400 MHz, DMSO-d₆) δ ppm 2.45 (s, 3H), 2.58 (s, 3H), 7.86 (d, J=1.0 Hz, 1H), 8.18 (d, J=1.0 Hz, 1H).

### Synthesis of tert-butyl (2S,4S)-4-(4-amino-1H-pyrazol-1-yl)-2-methylpiperidine-1-carboxylate (Int. D-2b)

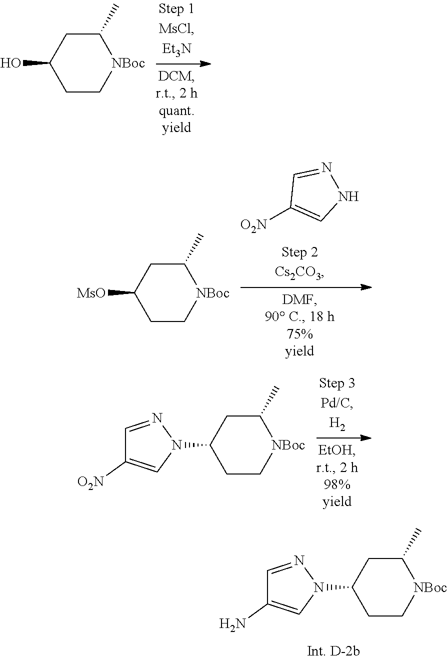 [SMILES: C[C@H]1C[C@H](O)CCN1C(=O)OC(C)(C)C ; O=[N+]([O-])c1cn[nH]c1 ; C[C@H]1C[C@H](OS(C)(=O)=O)CCN1C(=O)OC(C)(C)C ; C[C@H]1C[C@@H](n2cc([N+](=O)[O-])cn2)CCN1C(=O)OC(C)(C)C ; C[C@H]1C[C@@H](n2cc(N)cn2)CCN1C(=O)OC(C)(C)C]

Step 1: To a solution of tert-butyl (2S,4R)-4-hydroxy-2-methyl-piperidine-1-carboxylate 1 (500.0 mg, 2.32 mmol, 1 eq.) in CH₂Cl₂ (15 mL) at room temperature were added Et₃N (0.97 mL, 6.97 mmol, 3 equiv.) and MsCl (0.27 mL, 3.48 mmol, 1.5 equiv.). After 90 min., LCMS showed the expected mass. The reaction was quenched with saturated NH₄Cl solution and diluted with CH₂Cl₂. The phases were separated, and the organic layer was dried over sodium sulfate, filtered, and concentrated to give tert-butyl (2S,4R)-2-methyl-4-((methylsulfonyl)oxy)piperidine-1-carboxylate (701 mg, 95% yield) as a light-yellow solid.

LCMS: [M+H]⁺=194.2.

¹H NMR (400 MHz, chloroform-d) δ ppm 1.19 (d, J=7.1 Hz, 3H), 1.44-1.49 (m, 9H), 1.66 (br dd, J=12.1, 5.0 Hz, 1H), 1.83 (dt, J=12.1, 6.2 Hz, 1H), 1.99 (dt, J=12.6, 2.3 Hz, 1H), 2.07-2.21 (m, 1H), 2.93 (td, J=13.7, 2.7 Hz, 1H), 3.03 (s, 3H), 4.10 (br d, J=12.7 Hz, 1H), 4.48-4.63 (m, 1H), 4.89-5.02 (m, 1H).

Step 2: To a solution of tert-butyl (2S,4R)-2-methyl-4-((methylsulfonyl)oxy)piperidine-1-carboxylate (778 mg, 2.65 mmol, 1.2 eq.) and 4-nitro-1H-pyrazole 3 (250 mg, 2.21 mmol, 1 eq.) in DMF (10.2 mL) at room temperature was added Cs₂CO₃ (1.1 g, 3.1 mmol, 1.4 eq.). The reaction flask was placed in an oil bath pre-equilibrated to 90° C. After 18 h, LCMS showed full conversion. The reaction mixture was partitioned between water and EtOAc. The phases were separated, and the aqueous layer was extracted with EtOAc (3×). Combined organics were washed with brine (3×), dried over sodium sulfate, filtered, concentrated, and purified by reverse phase column chromatography (5% to 100% MeOH in water w/ 0.1% formic acid). The fractions were combined, concentrated to afford tert-butyl (2S,4S)-2-methyl-4-(4-nitro-1H-pyrazol-1-yl)piperidine-1-carboxylate (529 mg, 75% yield) as an orange oil.

LCMS: [M-tBu+H]⁺=255.2.

Step 3: To a solution of tert-butyl (tert-butyl (2S,4S)-2-methyl-4-(4-nitro-1H-pyrazol-1-yl)piperidine-1-carboxylate (529 mg, 1.68 mmol, 1 eq.) in Ethanol (8.5 mL) under nitrogen was added Pd/C (267.47 mg, 0.25 mmol, 0.15 eq.). The atmosphere of the flask was replaced with hydrogen by bubbling hydrogen into the mixture. Then the reaction mixture was stirred under 1 atm of hydrogen at room temperature. After 1.5 h, LCMS showed full conversion. The mixture was filtered through a Celite pad, washed with EtOAc, and concentrated under reduced pressure to give tert-butyl (2S,4S)-4-(4-amino-1H-pyrazol-1-yl)-2-methylpiperidine-1-carboxylate Int. D-2b (486 mg, 98% yield) as a purple oil.

LCMS: [M+H]⁺=281.4.

¹H NMR (400 MHz, DMSO-d₆) δ ppm 0.93 (d, J=6.6 Hz, 3H), 1.39-1.42 (m, 9H), 1.89-1.97 (m, 2H), 2.00-2.11 (m, 2H), 3.23-3.30 (m, 1H), 3.60-3.69 (m, 1H), 3.69-3.87 (m, 2H), 3.87-3.98 (m, 1H), 4.14-4.23 (m, 1H), 6.92 (s, 1H), 7.10 (s, 1H).

### Synthesis of tert-butyl (2R,4R)-4-(4-amino-1H-pyrazol-1-yl)-2-methylpiperidine-1-carboxylate (Int. D-2c)

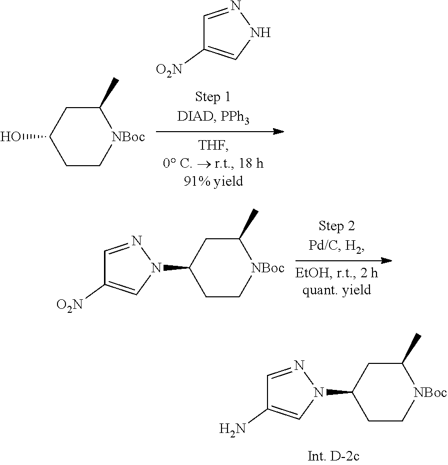 [SMILES: O=[N+]([O-])c1cn[nH]c1 ; C[C@@H]1C[C@@H](O)CCN1C(=O)OC(C)(C)C ; C[C@@H]1C[C@H](n2cc([N+](=O)[O-])cn2)CCN1C(=O)OC(C)(C)C ; C[C@@H]1C[C@H](n2cc(N)cn2)CCN1C(=O)OC(C)(C)C]

Step 1: To a solution of triphenylphosphine (1.50 g, 5.71 mmol 1.50 equiv.), tert-butyl (2R,4S)-4-hydroxy-2-methyl-piperidine-1-carboxylate (0.82 g, 3.8 mmol, 1.00 equiv.) and 4-nitro-1H-pyrazole (0.65 g, 5.71 mmol, 1.50 equiv.) in dry THF under argon (19.00 mL) at 0° C. was slowly added diisopropyl azodicarboxylate (1.12 mL, 5.71 mmol) in THF (19.00 mL, total concentration of 0.10 M), the reaction was brought to r.t. over the course of 18 h. The reaction was concentrated under reduced pressure and was purified by reverse phase column chromatography (5% to 90% MeOH in water w/ 0.1% formic acid) The fractions were combined and concentrated to afford tert-butyl (2R,4R)-2-methyl-4-(4-nitro-1H-pyrazol-1-yl)piperidine-1-carboxylate (1.14 g, 91% yield) as a colorless semi-solid.

LCMS: [M+H]⁺=211.2.

¹H NMR (400 MHz, DMSO-d₆) δ ppm 0.98 (d, J=6.6 Hz, 3H), 1.41 (s, 9H), 2.03-2.18 (m, 4H), 3.31 (ddd, J=14.2, 9.4, 5.5 Hz, 1H), 3.60-3.74 (m, 1H), 3.90-4.03 (m, 1H), 4.47-4.57 (m, 1H), 8.30 (s, 1H), 8.99 (s, 1H).

Step 2: To a solution of tert-butyl (2R,4R)-2-methyl-4-(4-nitro-1H-pyrazol-1-yl)piperidine-1-carboxylate (1.14 g, 3.49 mmol, 1.00 equiv.) in ethanol (34.89 mL, 0.10 M) was added 10% w/w Pd/C (371 mg, 0.35 mmol, 0.10 equiv.). H₂ gas was sparged into the mixture using a rubber balloon and a stainless needle. After 10 min of sparging, the exit needle was removed, and the reaction let to stir at r.t for 18 h under an H₂ atmosphere. LCMS showed full conversion to the desired product. The crude mixture was filtered on a 2-inch celite pad, which was washed further with MeOH. Concentration of the mixture afforded tert-butyl (2R,4R)-4-(4-amino-1H-pyrazol-1-yl)-2-methylpiperidine-1-carboxylate Int. D-2c (1.05 g, quant. yield) as a purple solid which was used without purification.

LCMS: [M+H]⁺=281.4.

¹H NMR (400 MHz, DMSO-d₆) δ ppm 0.93 (d, J=6.8 Hz, 3H), 1.40 (s, 9H), 1.87-2.10 (m, 4H), 3.22-3.31 (m, 1H), 3.60-3.69 (m, 1H), 3.75-3.86 (m, 2H), 3.93 (sxt, J=6.6 Hz, 1H), 4.13-4.25 (m, 1H), 6.92 (s, 1H), 7.10 (s, 1H).

### Synthesis of tert-butyl (3R,4R)-4-(4-amino-1H-pyrazol-1-yl)-3-fluoropiperidine-1-carboxylate (Int. D-2e)

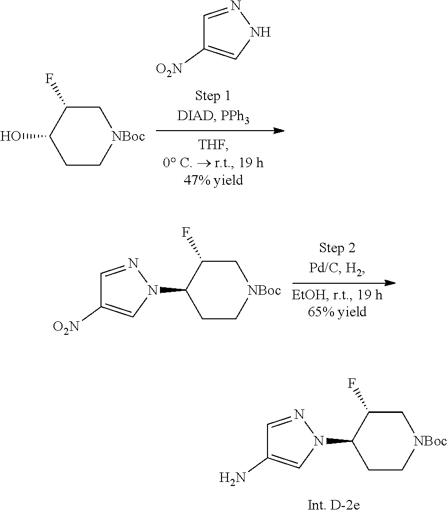 [SMILES: O=[N+]([O-])c1cn[nH]c1 ; CC(C)(C)OC(=O)N1CC[C@H](O)[C@H](F)C1 ; CC(C)(C)OC(=O)N1CC[C@@H](n2cc([N+](=O)[O-])cn2)[C@H](F)C1 ; CC(C)(C)OC(=O)N1CC[C@@H](n2cc(N)cn2)[C@H](F)C1]

Step 1: To a solution of 4-nitro-1H-pyrazole (618.9 mg, 5.47 mmol, 2 eq.), diisopropyl azodicarboxylate (1.1 mL, 5.47 mmol, 2 eq.) and triphenylphosphine polymer bound (1.82 g, 5.47 mmol, 2 eq.) in dry THE (7 mL, 0.2 M) at 0° C. under nitrogen was slowly added a solution of tert-butyl (3R,4S)-3-fluoro-4-hydroxy-piperidine-1-carboxylate (600 mg, 2.74 mmol, 1 eq.) in dry THF (7 mL, 0.2 M). After 19 h at room temperature, the reaction mixture was filtered and concentrated in vacuo and the crude residue was purified by reverse phase column chromatography (5% to 100% MeOH in water w/ 0.1% formic acid). The fractions were combined and concentrated to afford tert-butyl (3R,4R)-3-fluoro-4-(4-nitro-1H-pyrazol-1-yl)piperidine-1-carboxylate (404 mg, 47% yield) as a yellow oil.

LCMS: [M-tBu+H]⁺=259.2.

¹H NMR (400 MHz, DMSO-d₆) δ ppm 1.37 (d, J=6.4 Hz, 2H), 1.43 (s, 9H), 1.95 (qd, J=12.4, 4.4 Hz, 1H), 2.04-2.14 (m, 1H), 3.98 (br d, J=13.2 Hz, 1H), 4.22-4.39 (m, 1H), 4.68 (qd, J=10.7, 4.6 Hz, 1H), 4.74-4.95 (m, 1H), 8.35 (s, 1H), 9.09 (s, 1H).

¹⁹F NMR (377 MHz, DMSO-d₆) δ ppm −188.01 (br dd, J=50.4, 5.4 Hz, 1 F).

Step 2: A solution of tert-butyl (3R,4R)-3-fluoro-4-(4-nitro-1H-pyrazol-1-yl)piperidine-1-carboxylate (404 mg, 1.29 mmol, 1 eq.) in ethanol (23 mL, 0.06 M) was purged three times with nitrogen and Pd/C (137 mg, 0.13 mmol, 0.1 eq.) was added. The mixture was then purged three times with hydrogen gas (balloon) and then stirred for 19 h at room temperature under an atmosphere of hydrogen. The mixture was filtered on a pad of Celite and rinsed with EtOAc and EtOH. The filtrate was concentrated in vacuo to give afford tert-butyl (3R,4R)-4-(4-amino-1H-pyrazol-1-yl)-3-fluoropiperidine-1-carboxylate Int. D-2e (370 mg, 65% yield) as a purple oil which was used without further purification.

LCMS: [M-t-Bu+H]⁺=229.2.

¹H NMR (400 MHz, DMSO-d₆) δ ppm 1.18 (d, J=6.1 Hz, 2H), 1.42 (s, 9H), 1.80-1.97 (m, 2H), 3.77-3.97 (m, 2H), 4.22-4.37 (m, 2H), 4.57-4.83 (m, 2H), 6.95-7.02 (m, 1H), 7.11-7.20 (m, 1H).

¹⁹F NMR (377 MHz, DMSO-d₆) δ ppm −186.81-−186.56 (m, 1 F).

### Synthesis of tert-butyl (3R,4S)-4-(4-amino-1H-pyrazol-1-yl)-3-fluoropiperidine-1-carboxylate (Int. D-2f)

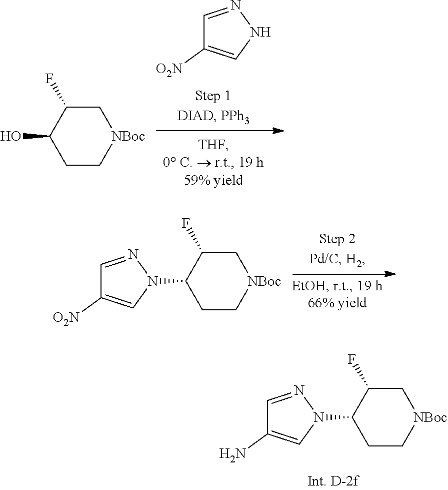 [SMILES: O=[N+]([O-])c1cn[nH]c1 ; CC(C)(C)OC(=O)N1CC[C@@H](O)[C@H](F)C1 ; CC(C)(C)OC(=O)N1CC[C@H](n2cc([N+](=O)[O-])cn2)[C@H](F)C1 ; CC(C)(C)OC(=O)N1CC[C@H](n2cc(N)cn2)[C@H](F)C1]

Step 1: To a solution of 4-nitro-1H-pyrazole (645 mg, 5.71 mmol), diisopropyl azodicarboxylate (1.1 mL, 5.49 mmol) and triphenylphosphine polymer bound (1.98 g, 5.94 mmol) in dry THF (18 mL) at 0° C. under nitrogen was slowly added a solution of tert-butyl (3R,4R)-3-fluoro-4-hydroxy-piperidine-1-carboxylate (607 mg, 2.77 mmol) in dry THF (18 mL). After 18 h at room temperature, the reaction mixture was filtered and concentrated in vacuo. The residue was purified by reverse phase column chromatography (5% to 100% MeOH in water w/0.1% formic acid). The fractions were collected, affording tert-butyl (3R,4S)-3-fluoro-4-(4-nitro-1H-pyrazol-1-yl)piperidine-1-carboxylate (511 mg, 1.63 mmol, 59% yield) as a white solid.

LCMS: [M-tBu+H]⁺=259.2.

¹H NMR (400 MHz, DMSO-d₆) δ ppm 1.19 (br d, J=6.1 Hz, 1H), 1.42 (s, 9H), 1.99-2.08 (m, 1H), 2.24-2.36 (m, 1H), 2.84-3.10 (m, 1H), 4.09-4.22 (m, 1H), 4.24-4.43 (m, 1H), 4.68-4.83 (m, 1H), 4.99-5.19 (m, 1H), 8.35 (s, 1H), 8.95 (s, 1H).

¹⁹F NMR (377 MHz, DMSO-d₆) δ ppm −203.26-−202.52 (m, 1 F).

The mixture was then purged three times with hydrogen gas (balloon) and then stirred 19 h at room temperature under hydrogen (balloon). The mixture was flushed with nitrogen and filtered on a pad of Celite and rinsed with MeOH (50 mL). The filtrate was concentrated in vacuo to afford tert-butyl (3R,4S)-4-(4-amino-1H-pyrazol-1-yl)-3-fluoropiperidine-1-carboxylate Int. D-2f (453 mg, 1.05 mmol, 66% yield) as a purple oil, which was used without further purification.

LCMS: [M+H]⁺=285.2.

¹H NMR (400 MHz, DMSO-d₆) δ ppm 1.14-1.27 (m, 1H), 1.41 (s, 9H), 1.81-1.93 (m, 1H), 1.99-2.18 (m, 1H), 2.79-3.01 (m, 1H), 4.12 (br d, J=2.9 Hz, 1H), 4.18-4.31 (m, 1H), 4.36-4.48 (m, 1H), 4.67-4.82 (m, 2H), 4.84-5.07 (m, 1H), 7.02 (s, 1H), 7.12-7.17 (m, 1H).

¹⁹F NMR (377 MHz, DMSO-d₆) δ ppm −202.38-−201.24 (m, 1 F).

### Synthesis of tert-butyl (3S,4R)-4-(4-amino-1H-pyrazol-1-yl)-3-fluoropiperidine-1-carboxylate (Int. D-2h)

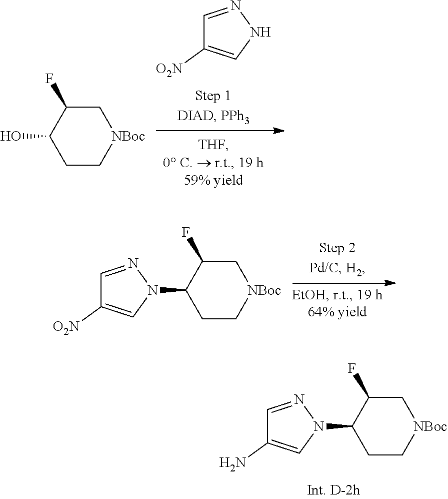 [SMILES: O=[N+]([O-])c1cn[nH]c1 ; CC(C)(C)OC(=O)N1CC[C@H](O)[C@@H](F)C1 ; CC(C)(C)OC(=O)N1CC[C@@H](n2cc([N+](=O)[O-])cn2)[C@@H](F)C1 ; CC(C)(C)OC(=O)N1CC[C@@H](n2cc(N)cn2)[C@@H](F)C1]

Step 1: A solution of 4-nitro-1H-pyrazole (643 mg, 5.69 mmol), tert-butyl (3S,4S)-3-fluoro-4-hydroxy-piperidine-1-carboxylate (604 mg, 2.76 mmol) and triphenylphosphine polymer bound (1.91 g, 5.74 mmol) in dry THF (18 mL) was prepared and cooled 0° C. under nitrogen. A solution of diisopropyl azodicarboxylate (1.1 mL, 5.59 mmol) in dry THF (18 mL) was then added slowly. After 17 h at room temperature, the reaction mixture was filtered, concentrated, and purified by reverse phase column chromatography (5% to 100% MeOH in water w/ 0.1% formic acid). The fractions were collected, affording tert-butyl (3S,4R)-3-fluoro-4-(4-nitro-1H-pyrazol-1-yl)piperidine-1-carboxylate (511 mg, 1.62 mmol, 59% yield) as a white solid.

LCMS: [M-tBu+H]⁺=259.2.

¹H NMR (400 MHz, DMSO-d₆) δ ppm 1.19-1.24 (m, 1H), 1.41 (s, 9H), 2.01 (br dd, J=12.7, 3.2 Hz, 1H), 2.22-2.37 (m, 1H), 3.15-3.36 (m, 1H), 4.15 (br s, 1H), 4.22-4.38 (m, 1H), 4.68-4.81 (m, 1H), 5.00-5.18 (m, 1H), 8.34 (s, 1H), 8.94 (s, 1H).

¹⁹F NMR (377 MHz, DMSO-d₆) δ ppm −205.91-−197.72 (m, 1 F).

Step 2: A solution of tert-butyl (3S,4R)-3-fluoro-4-(4-nitro-1H-pyrazol-1-yl)piperidine-1-carboxylate (511 mg, 1.62 mmol) in ethanol (16 mL) was purged three times with nitrogen and Pd/C (187.0 mg, 0.18 mmol) was added. The mixture was then purged three times with hydrogen gas (balloon) and then stirred 22 h at rt under hydrogen (balloon). The mixture was flushed with nitrogen and filtered on a pad of Celite and rinsed with MeOH (50 mL). The filtrate was concentrated in vacuo to afford tert-butyl (3S,4R)-4-(4-amino-1H-pyrazol-1-yl)-3-fluoropiperidine-1-carboxylate (427 mg, 1.05 mmol, 64% yield) as a red oil, which was used without further purification.

LCMS: [M+H]⁺=285.2.

¹H NMR (400 MHz, DMSO-d₆) δ ppm 1.21-1.28 (m, 1H), 1.41 (s, 9H), 1.85 (br dd, J=12.8, 3.1 Hz, 1H), 2.07 (qd, J=12.7, 4.7 Hz, 1H), 3.05-3.25 (m, 1H), 4.05-4.13 (m, 1H), 4.20-4.28 (m, 1H), 4.30-4.48 (m, 1H), 4.81-5.02 (m, 1H), 6.95 (s, 1H), 7.04 (s, 1H). Two protons not observed.

¹⁹F NMR (377 MHz, DMSO-d₆) δ ppm −202.48-−200.97 (m, 1 F).

### Synthesis of tert-butyl (3S,4S)-4-(4-amino-1H-pyrazol-1-yl)-3-fluoropiperidine-1-carboxylate (Int. D-2i)

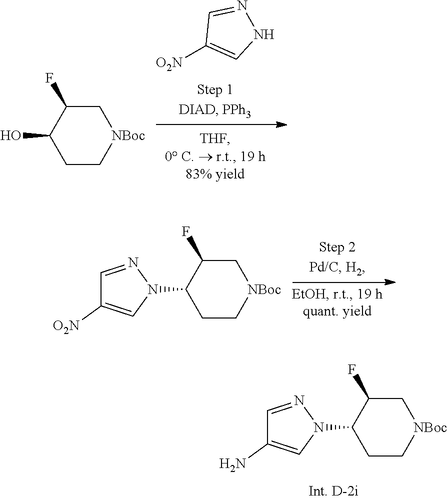 [SMILES: O=[N+]([O-])c1cn[nH]c1 ; CC(C)(C)OC(=O)N1CC[C@@H](O)[C@@H](F)C1 ; CC(C)(C)OC(=O)N1CC[C@H](n2cc([N+](=O)[O-])cn2)[C@@H](F)C1 ; CC(C)(C)OC(=O)N1CC[C@H](n2cc(N)cn2)[C@@H](F)C1]

Step 1: A solution of 4-nitro-1H-pyrazole (414 mg, 3.66 mmol), tert-butyl (3S,4R)-3-fluoro-4-hydroxy-piperidine-1-carboxylate (375 mg, 1.71 mmol) and triphenylphosphine polymer bound (1.31 g, 3.93 mmol) in dry THF (18 mL) was prepared and cooled to at 0° C. under nitrogen. A solution of diisopropyl azodicarboxylate (0.66 mL, 3.34 mmol) in dry THF (18 mL) was added slowly. After 18 h at rt, the reaction mixture was filtered, concentrated, and purified by reverse phase column chromatography (5% to 100% MeOH in water w/0.1% formic acid) affording tert-butyl (3S,4S)-3-fluoro-4-(4-nitro-1H-pyrazol-1-yl)piperidine-1-carboxylate (452 mg, 1.42 mmol, 83% yield) as a yellow solid.

LCMS: [M-tBu+H]⁺=259.2.

¹H NMR (400 MHz, DMSO-d₆) δ ppm 1.21-1.25 (m, 1H), 1.43 (s, 9H), 1.88-2.04 (m, 1H), 2.08-2.13 (m, 1H), 3.98 (br d, J=12.6 Hz, 1H), 4.22-4.39 (m, 1H), 4.62-4.74 (m, 1H), 4.75-4.83 (m, 1H), 4.84-4.96 (m, 1H), 8.35 (s, 1H), 9.09 (s, 1H).

¹⁹F NMR (377 MHz, DMSO-d₆) δ ppm −188.02 (br dd, J=49.0, 5.5 Hz, 1 F).

Step 2: A solution of tert-butyl (3S,4S)-3-fluoro-4-(4-nitro-1H-pyrazol-1-yl)piperidine-1-carboxylate (441 mg, 1.40 mmol) in ethanol (12 mL) was purged three times with nitrogen and Pd/C (20.2 mg, 0.20 mmol) was added. The mixture was then purged three times with hydrogen gas (balloon) and then stirred 18 h at rt under hydrogen (balloon). As no conversion was observed, the reaction mixture was purged three times with nitrogen and more Pd/C (54.6 mg, 0.5100 mmol) was added. The mixture was then purged three times with hydrogen gas (balloon) and then stirred 17 h at rt under hydrogen (balloon). The mixture was flushed with nitrogen and filtered on a pad of Celite and rinsed with MeOH (100 mL). The filtrate was concentrated to afford tert-butyl (3S,4S)-4-(4-amino-1H-pyrazol-1-yl)-3-fluoropiperidine-1-carboxylate (425 mg, 1.50 mmol, quant. Yield) as a red oil, which was used without further purification.

LCMS: [M-tBu+H]⁺=229.2.

¹H NMR (400 MHz, DMSO-d₆) δ ppm 1.14-1.17 (m, 1H), 1.41 (s, 9H), 1.82-1.92 (m, 2H), 3.81 (br s, 2H), 3.91 (br d, J=13.0 Hz, 2H), 4.29-4.38 (m, 1H), 4.54-4.66 (m, 1H), 4.68-4.78 (m, 1H), 6.96 (s, 1H), 7.12 (s, 1H).

¹⁹F NMR (377 MHz, DMSO-d₆) δ ppm −186.83-−186.56 (m, 1 F).

### Synthesis of tert-butyl (S)-4-(4-amino-1H-pyrazol-1-yl)azepane-1-carboxylate (Int. D-2j) and tert-butyl (R)-4-(4-amino-1H-pyrazol-1-yl)azepane-1-carboxylate (Int. D-2k)

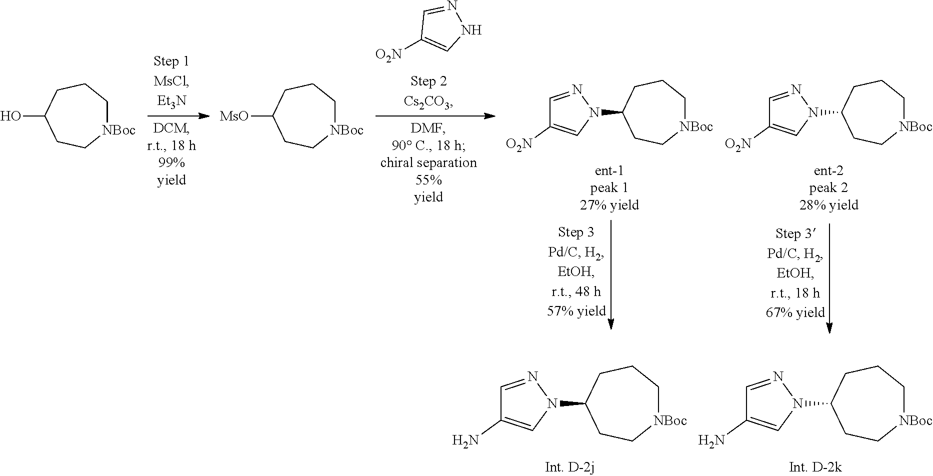

Step 1: Triethylamine (2.59 mL, 18.58 mmol, 4 eq.) was added to a solution of tert-butyl 4-hydroxyazepane-1-carboxylate 1 (1.0 g, 4.64 mmol, 1 eq.) in DCM (15.48 mL). The flask was cooled to 0° C. and methanesulfonyl chloride (1.08 mL, 13.93 mmol, 3 eq.) was added dropwise. The reaction mixture was stirred at room temperature under a nitrogen atmosphere for 18 h. After stirring for 18 h, the reaction was quenched with water (50 mL) and the organic layer was extracted three times with DCM and washed with brine, dried over Na₂SO₄ and evaporated under vacuum to afford tert-butyl 4-((methylsulfonyl)oxy)azepane-1-carboxylate (1.35 g, 99% yield) as an orange oil, which was used without further purification.

¹H NMR (400 MHz, CDCl₃) δ ppm 1.46 (s, 9H), 1.60-1.75 (m, 2H), 1.94-2.10 (m, 4H), 3.02 (s, 3H), 3.32-3.56 (m, 4H), 4.84-4.97 (m, 1H).

Step 2: To a round-bottom flask charged with 4-nitro-1H-pyrazole (0.51 g, 4.51 mmol, 1 eq.) was added a solution of tert-butyl 4-((methylsulfonyl)oxy)azepane-1-carboxylate (1.32 g, 4.51 mmol, 1 eq.) in DMF (10.25 mL). CS₂CO₃ (2.94 g, 9.02 mmol, 2 eq.) was then added and the reaction mixture was stirred at 90° C. for 18 h under a nitrogen atmosphere. After heating for 18 h, LCMS showed complete conversion. The reaction was cooled to room temperature and water was added. The aqueous layer was extracted with EtOAc (3×), the combined organic layers were washed with brine, dried over Na₂SO₄ and concentrated to dryness to give an orange oil which was purified by silica gel column chromatography (0% to 100% EtOAc in heptane). The fractions were combined and concentrated to afford tert-butyl (rac)-4-(4-nitro-1H-pyrazol-1-yl)azepane-1-carboxylate (0.873 g) as a yellow oil. The material was then separated by chiral SFC to afford the two enantiomers, ent-1 (assigned as (S), 0.381 g, 27% yield) as a light-yellow oil and ent-2 (assigned as (R), 0.393 g, 28% yield) as a light-yellow oil. NOTE: the absolute stereochemistry was arbitrarily assigned from the first and second eluting enantiomers of the chiral separation.

ent-1: LCMS: [M-Boc+H]⁺=211.2. ¹H NMR (400 MHz, DMSO-d₆) δ ppm 1.42 (s, 9H), 1.58-1.73 (m, 1H), 1.79-1.90 (m, 1H), 1.90-2.03 (m, 2H), 2.07 (s, 2H), 3.20-3.29 (m, 1H), 3.34-3.42 (m, 2H), 3.50-3.66 (m, 1H), 4.35-4.48 (m, 1H), 8.26 (s, 1H), 8.92 (s, 1H).

ent-2: LCMS: [M-boc+H]⁺=211.2. ¹H NMR (400 MHz, DMSO-d₆) δ ppm 1.42 (s, 9H), 1.58-1.73 (m, 1H), 1.79-1.90 (m, 1H), 1.90-2.03 (m, 2H), 2.07 (s, 2H), 3.20-3.29 (m, 1H), 3.34-3.42 (m, 2H), 3.50-3.66 (m, 1H), 4.35-4.48 (m, 1H), 8.26 (s, 1H), 8.92 (s, 1H).

Step 3: To a solution of tert-butyl (S)-4-(4-nitro-1H-pyrazol-1-yl)azepane-1-carboxylate ent-1 (0.33 g, 1.05 mmol, 1.0 eq.) in ethanol (10.47 mL, 0.1 M) under nitrogen was added Pd/C (167 mg, 0.16 mmol, 0.2 eq.). The atmosphere of the flask was replaced with hydrogen by bubbling into the mixture for 10 min. The reaction mixture was stirred under 1 atm of hydrogen at room temperature for 48 h. The mixture was filtered through a Celite pad and washed with EtOAc. The filtrate was then dried in vacuo and purified by silica gel column chromatography (0% to 100% EtOAc in heptane). The fractions were combined and concentrated to afford tert-butyl (S)-4-(4-amino-1H-pyrazol-1-yl)azepane-1-carboxylate Int. D-2j (185 mg, 57% yield) as a red solid.

LCMS: [M+H]⁺=281.2.

¹H NMR (400 MHz, DMSO-d₆) δ ppm 1.41 (s, 9H), 1.53-1.70 (m, 1H), 1.72-1.86 (m, 3H), 1.91-2.04 (m, 3H), 3.14-3.26 (m, 2H), 3.46-3.58 (m, 1H), 3.72-3.82 (m, 1H), 4.02-4.11 (m, 1H), 6.88 (s, 1H), 7.00 (d, J=0.7 Hz, 1H). One proton is not apparent in the ¹H NMR spectrum.

Step 3′: To a solution of tert-butyl (R)-4-(4-nitro-H-pyrazol-1-yl)azepane-1-carboxylate ent-2 (0.38 g, 1.23 mmol) in ethanol (12.3 mL) under nitrogen was added Pd/C (394 mg, 0.37 mmol). The atmosphere of the flask was replaced with hydrogen by bubbling hydrogen into the mixture. The reaction mixture was stirred under 1 atm of hydrogen at room temperature overnight. The mixture was filtered trough a Celite pad, and the pad was rinsed with EtOAc. The filtrate was then passed over silica pad. The filtrate was then concentrated affording tert-butyl (R)-4-(4-amino-H-pyrazol-1-yl)azepane-1-carboxylate nt. D-2k (285 mg 670 yield) as a red solid, which was used without further purification.

LCMS: [M+H]⁺=281.2.

¹H NMR (400 MHz, DMSO-d₆) δ ppm 1.42 (s, 9H), 1.58-1.68 (m, 2H), 1.72-1.89 (m, 4H), 3.17-3.26 (m, 2H), 3.47-3.59 (m, 2H), 4.04-4.13 (m, 1H), 6.88 (s, 1H), 7.01-7.02 (m, 1H).

TABLE 17
List of Int. D-3 compounds generated by General Procedure TBM-D: Step 1

| Starting | | | | |
|---|---|---|---|---|
| Materials | | Product | | |
| Int. | Int. | Int. | | |
| D-1 | D-2 | D-3 | Structure | Characterization |
| Int. D-1a | Int. D-2a | Int. D-3a | 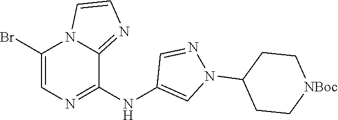 [SMILES: CC(C)(C)OC(=O)N1CCC(n2cc(Nc3ncc(Br)n4ccnc34)cn2)CC1] | 35% yield, tan solid LCMS Method 1: [M]⁺ 462.3, rt: 1.66 min. |
| Int. D-1b | Int D-2a | Int. D-3b | 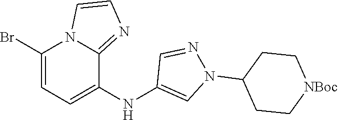 [SMILES: CC(C)(C)OC(=O)N1CCC(n2cc(Nc3ccc(Br)n4ccnc34)cn2)CC1] | 87% yield, red solid [M + H]⁺ = 417.2 ¹H NMR (400 MHz, DMSO-d₆) δ ppm 1.42 (s, 9 H) 1.80 (qd, J =12.19, 4.52 Hz, 2 H) 2.00 (d, J = 11.00 Hz, 2 H) 2.91 (br. S., 2 H) 4.00-4.10 (m, 2 H) 4.27-4.37 (m, 1 H) 6.48 (d, J = 8.07 Hz, 1 H) 6.91 (d, J = 8.07 Hz, 1 H) 7.53 (s, 1 H) 7.63 (s, 1 H) 7.91 (d, J = 4.80 Hz, 2 H) 8.16 (s, 1 H) |
| Int. D-1a | Int D-2b | Int. D-3c | 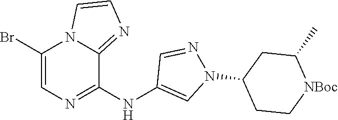 [SMILES: C[C@H]1C[C@@H](n2cc(Nc3ncc(Br)n4ccnc34)cn2)CCN1C(=O)OC(C)(C)C] | 76% yield, light-yellow solid [M + H]⁺ = 476.2, 478.2 ¹H NMR (400 MHz, DMSO-d₆) δ ppm 0.99 (d, J = 6.6 Hz, 3 H), 1.41 (s, 9 H), 1.97-2.16 (m, 4 H), 3.33-3.41 (m, 1 H), 3.63-3.72 (m, 1 H), 3.91-4.01 (m, 1 H), 4.32-4.46 (m, 1 H), 7.59 (s, 1 H), 7.70 (s, 1 H), 7.78 (s, 1 H), 7.98 (s, 1 H), 8.22 (s, 1 H), 10.01 (s, 1 H). |
| Int. D-1a | Int. D-2c | Int. D-3d | 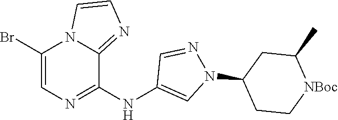 [SMILES: C[C@@H]1C[C@H](n2cc(Nc3ncc(Br)n4ccnc34)cn2)CCN1C(=O)OC(C)(C)C] | 66% yield, tan solid [M + H]⁺ = 476.2, 478.2 ¹H NMR (400 MHz, DMSO-d₆) δ ppm 0.98 (d, J = 6.6 Hz, 3 H), 1.41 (s, 9 H), 1.94-2.19 (m, 4 H), 3.36 (td, J = 9.4, 5.0 Hz, 1 H), 3.68 (dt, J = 13.5, 4.7 Hz, 1 H), 3.96 (sxt, J = 6.6 Hz, 1 H), 4.35- 4.46 (m, 1 H), 7.59 (s, 1 H), 7.70 (s, 1 H), 7.78 (s, 1 H), 7.99 (s, 1 H), 8.22 (s, 1 H), 10.01 (s, 1 H). |
| Int. D-1a | Int. D-2d | Int. D-3e | 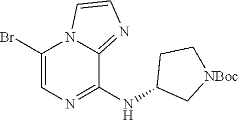 [SMILES: CC(C)(C)OC(=O)N1CC[C@@H](Nc2ncc(Br)n3ccnc23)C1] | 54% yield, red solid [M + H]⁺ = 382.0 ¹H NMR (400 MHz, DMSO-d₆) δ ppm 1.39 (br d, J = 6.8 Hz, 9 H), 2.00-2.20 (m, 2 H), 3.23-3.31 (m, 2 H), 3.40- 3.50 (m, 1 H), 3.53-3.64 (m, 1 H), 4.49- 4.63 (m, 1 H), 7.50 (s, 1 H), 7.64 (s, 1 H), 7.86 (br s, 1 H), 7.93 (d, J = 1.0 Hz, 1 H). |
| Int. D-1c | Int. D-2a | Int. D-3f | 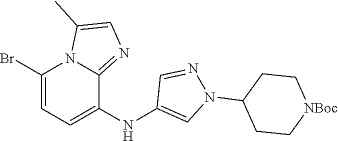 [SMILES: Cc1cnc2c(Nc3cnn(C4CCN(C(=O)OC(C)(C)C)CC4)c3)ccc(Br)n12] | 24% yield, brown solid [M + H]⁺ = 476.0, 478.0 ¹H NMR (400 MHz, DMSO-d₆) δ ppm 1.41-1.43 (m, 9 H), 1.68-1.82 (m, 2 H), 1.94-2.02 (m, 2 H), 2.71-2.77 (m, 3 H), 3.99-4.13 (m, 4 H), 4.28-4.38 (m, 1 H), 7.39-7.42 (m, 1 H), 7.42- 7.45 (m, 1 H), 7.72-7.78 (m, 1 H), 8.15 (s, 1 H), 9.82-9.87 (m, 1 H). |
| Int. D-1a | Int. D-2e | Int. D-3g | 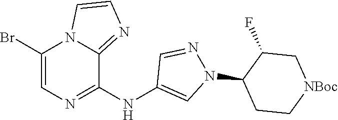 [SMILES: CC(C)(C)OC(=O)N1CC[C@@H](n2cc(Nc3ncc(Br)n4ccnc34)cn2)[C@H](F)C1] | 38% yield, brown solid [M+H] = 480.0; 482.0 ¹H NMR (400 MHz, DMSO-d₆) δ ppm 1.43 (s, 9 H), 1.86-2.07 (m, 2 H), 2.84- 3.12 (m, 2 H), 3.91-4.04 (m, 1 H), 4.29 (br s, 1 H), 4.51-4.62 (m, 1 H), 4.65-4.90 (m, 1 H), 7.56-7.61 (m, 1 H), 7.69-7.72 (m, J = 1.0 Hz, 1 H), 7.82 (s, 1 H), 7.99 (s, 1 H), 8.27 (s, 1 H), 10.05 (s, 1 H). ¹⁹F NMR (377 MHz, DMSO-d₆) δ ppm −186.86-−186.61 (m, 1 F). |
| Int. D-1a | Int. D-2f | Int. D-3h | 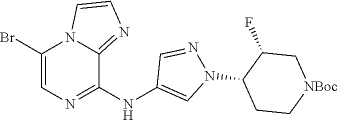 [SMILES: CC(C)(C)OC(=O)N1CC[C@H](n2cc(Nc3ncc(Br)n4ccnc34)cn2)[C@H](F)C1] | 35% yield, orange oil [M + H]⁺ = 480.0 ¹H NMR (400 MHz, DMSO-d₆) δ ppm 1.42 (s, 9 H), 1.93-2.03 (m, 1 H), 2.12- 2.23 (m, 1 H), 2.85-3.07 (m, 1 H), 3.08-3.25 (m, 1 H), 4.07-4.22 (m, 1 H), 4.21-4.41 (m, 1 H), 4.53-4.71 (m, 1 H), 4.89-5.12 (m, 1 H), 7.62 (s, 1 H), 7.71 (d, J = 1.1 Hz, 1 H), 7.82 (s, 1 H), 7.99 (d, J = 1.2 Hz, 1 H), 8.20 (s, 1 H), 10.04 (s, 1 H). ¹⁹F NMR (377 MHz, DMSO-d₆) δ ppm −202.53-−200.95 (m, 1 F). |
| Int D-1a | Int. D-2g | Int. D-3i | 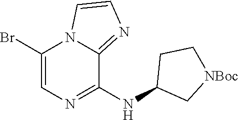 [SMILES: CC(C)(C)OC(=O)N1CC[C@H](Nc2ncc(Br)n3ccnc23)C1] | 54% yield, brown solid [M + H]⁺ = 382.0 ¹H NMR (400 MHz, DMSO-d₆) δ ppm 1.39 (br d, J = 6.8 Hz, 9 H), 1.99-2.20 (m, 2 H), 3.22-3.30 (m, 2 H), 3.40- 3.49 (m, 1 H), 3.53-3.63 (m, 1 H), 4.48- 4.64 (m, 1 H), 7.51 (s, 1 H), 7.64 (d, J = 0.7 Hz, 1 H), 7.83-7.90 (m, 1 H), 7.93 (d, J = 1.0 Hz, 1 H). |
| Int. D-1a | Int. D-2h | Int. D-3j | 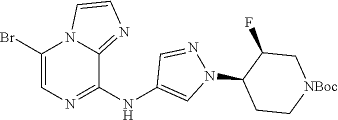 [SMILES: CC(C)(C)OC(=O)N1CC[C@@H](n2cc(Nc3ncc(Br)n4ccnc34)cn2)[C@@H](F)C1] | 43% yield, brown oil [M + H]⁺ = 480.0 ¹H NMR (400 MHz, DMSO-d₆) δ ppm 1.42 (s, 9 H), 1.89-2.01 (m, 1 H), 2.11- 2.22 (m, 1 H), 2.86-3.23 (m, 2 H), 4.11-4.21 (m, 1 H), 4.23-4.36 (m, 1 H), 4.53-4.68 (m, 1 H), 4.90-5.08 (m, 1 H), 7.61 (s, 1 H), 7.71 (d, J = 1.0 Hz, 1 H), 7.81 (s, 1 H), 7.97-8.01 (m, 1 H), 8.19 (s, 1 H), 10.04 (s, 1 H). ¹⁹F NMR (377 MHz, DMSO-d₆) δ ppm −202.60-−201.25 (m, 1 F). |
| Int D-1a | Int. D-2i | Int. D-3k | 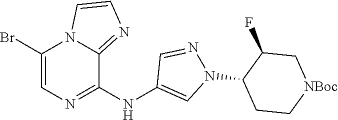 [SMILES: CC(C)(C)OC(=O)N1CC[C@H](n2cc(Nc3ncc(Br)n4ccnc34)cn2)[C@@H](F)C1] | 46% yield, brown oil [M + H]⁺ = 480.0. ¹H NMR (400 MHz, DMSO-d₆) δ ppm 1.17-1.24 (m, 1 H), 1.43 (s, 9 H), 1.89- 1.97 (m, 1 H), 3.28-3.36 (m, 1 H), 3.97 (br d, J = 12.8 Hz, 1 H), 4.22-4.33 (m, 1 H), 4.48-4.63 (m, 1 H), 4.65- 4.76 (m, 1 H), 4.76-4.96 (m, 1 H), 7.60 (s, 1 H), 7.71 (d, J = 1.2 Hz, 1 H), 7.83 (s, 1 H), 7.99 (d, J = 1.1 Hz, 1 H), 8.27 (s, 1 H), 10.05 (s, 1 H). ¹⁹F NMR (377 MHz, DMSO-d₆) δ ppm −186.83-−186.58 (m, 1 F). |
| Int D-1d | Int. D-2a | Int. D-31 | 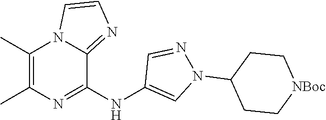 [SMILES: Cc1nc(Nc2cnn(C3CCN(C(=O)OC(C)(C)C)CC3)c2)c2nccn2c1C] | 85% yield, brown foam [M + H]⁺ = 412.2 ¹H NMR (400 MHz, DMSO-d₆) δ ppm 1.42 (s, 9 H), 1.76 (qd, J = 12.1, 4.2 Hz, 2 H), 1.99 (br d, J = 11.2 Hz, 2 H), 2.37 (s, 3 H), 2.44 (s, 3 H), 2.79-3.05 (m, 2 H), 4.04 (br d, J = 12.2 Hz, 2 H), 4.22- 4.43 (m, 1 H), 7.58 (d, J = 1.0 Hz, 1 H), 7.79 (s, 1 H), 7.84 (d, J = 0.7 Hz, 1 H), 8.23 (s, 1 H), 9.54 (s, 1 H). |
| Int. D-1a | Int D-2j | Int. D-3m | 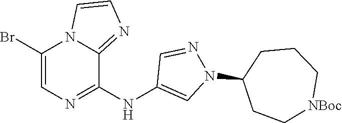 [SMILES: CC(C)(C)OC(=O)N1CCC[C@H](n2cc(Nc3ncc(Br)n4ccnc34)cn2)CC1] | 31% yield, brown solid [M + H]⁺ = 476.0, 478.0. ¹H NMR (400 MHz, DMSO-d₆) δ ppm 1.43 (s, 9 H), 1.62-1.72 (m, 1 H), 1.81- 2.07 (m, 5 H), 3.16-3.25 (m, 1 H), 3.36-3.43 (m, 2 H), 3.53-3.66 (m, 1 H), 4.24-4.35 (m, 1 H), 7.59 (d, J = 3.7 Hz, 1 H), 7.70 (d, J = 1.2 Hz, 1 H), 7.74 (s, 1 H), 7.98 (d, J = 1.2 Hz, 1 H), 8.14 (s, 1 H), 10.00 (s, 1 H). |
| Int. D-1a | Int. D-2k | Int. D-3n | 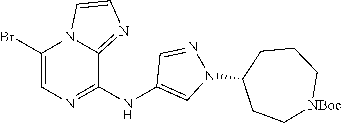 [SMILES: CC(C)(C)OC(=O)N1CCC[C@@H](n2cc(Nc3ncc(Br)n4ccnc34)cn2)CC1] | 36% yield, brown solid [M + H]⁺ = 476.0 ¹H NMR (400 MHz, DMSO-d₆) δ ppm 1.43-1.45 (m, 9 H), 1.63-1.74 (m, 1 H), 1.80-1.91 (m, 2 H), 1.93-2.07 (m, 3 H), 3.20-3.29 (m, 1 H), 3.37-3.43 (m, 2 H), 3.53-3.67 (m, 1 H), 4.25- 4.34 (m, 1 H), 7.58-7.61 (m, 1 H), 7.71 (d, J = 1.0 Hz, 1 H), 7.75 (s, 1 H), 7.99 (d, J = 1.0 Hz, 1 H), 8.15 (s, 1 H), 10.01 (s, 1 H). |

*Note: Buchwald-Hartwig coupling done w/: Pd(Oac)₂, rac-BINAP, Cs₂CO₃, toluene, 120° C., 4 h

### Synthesis of TBM-D: Step 2

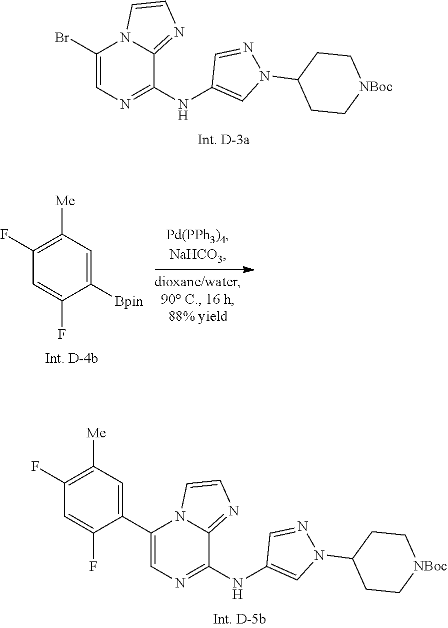

To a solution of 2-(2,4-difluoro-5-methyl-phenyl)-4,4,5,5-tetramethyl-1,3,2-dioxaborolane Int. D-4b (2.37 g, 8.76 mmol, 1.5 equiv.) and tert-butyl 4-[4-[(5-bromoimidazo[1,2-a]pyrazin-8-yl)amino]pyrazol-1-yl]piperidine-1-carboxylate Int. D-3a (2.7 g, 5.84 mmol, 1 equiv.) in 1,4-dioxane (21.2 mL, 0.2 M) in a sealed tube was added NaHCO₃ (1.47 g, 17.52 mmol, 3 equiv.) and water (9.7 mL, 0.2 M), then N₂ was bubbled for 5 min. Then, Pd(PPh₃)₄ (675 mg, 0.58 mmol, 0.1 equiv.) was added and N₂ was bubbled for 10 min. The resulting solution was stirred at 90° C. in a sealed tube under N₂ atmosphere for 16 h. The reaction mixture was cooled down to room temperature, water and EtOAc were added to the solution and the phases were separated. The aqueous phase was extracted with EtOAc (3×). The combined organic layers were dried over Na₂SO₄, filtered, and evaporated to give a yellow oil. The yellow oil was then purified by reverse phase column chromatography (500 to 10000 MeOH in water w/ 0.1% formic acid). The fractions were combined and concentrated to afford tert-butyl 4-(4-((5-(2,4-difluoro-5S-methylphenyl)imidazo[1,2-a]pyrazin-8-yl)amino)-1H-pyrazol-1-yl)piperidine-1-carboxylate Int. D-5b (2.65 g, 880 yield) as a light orange solid.

LCMS: [M+H]⁺=510.2.

¹H NMR (400 MHz, DMSO-d₆) δ ppm 1.42 (s, 9H), 1.71-1.84 (m, 2H), 1.97-2.04 (m, 2H), 2.29 (s, 3H), 3.15-3.19 (m, 1H), 3.98-4.14 (m, 3H), 4.31-4.41 (m, 1H), 7.41-7.48 (m, 2H), 7.58-7.63 (m, J=5.6 Hz, 3H), 7.81 (s, 1H), 8.25 (s, 1H), 9.99 (s, 1H).

TABLE 18
List of intermediates used in TBM-D: Step 2

| Int. D-4 | Structure | Source |
|---|---|---|
| Int. D-4a | 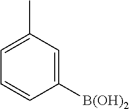 | commercially available |
| Int. D-4b | 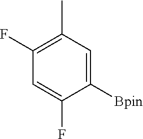 | commercially available |
| Int. D-4c | 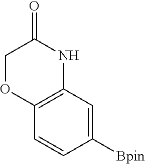 | commercially available |
| Int. D-4d | 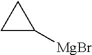 | commercially available |
| Int. D-4e | 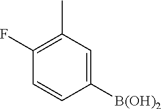 | commercially available |
| Int. D-4f | 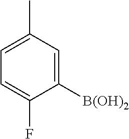 | commercially available |
| Int. D-4g | 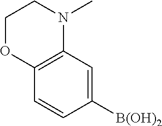 | commercially available |
| Int. D-4 h | 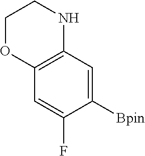 | commercially available |
| Int. D-4i | 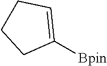 | commercially available |
| Int. D-4j | 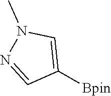 | commercially available |
| Int. D-4k | 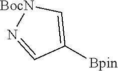 | commercially available |
| Int. D-41 | 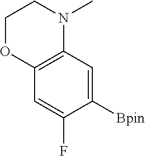 | synthesis provided below |
| Int. D-4m | 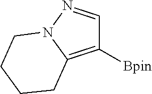 | commercially available |
| Int. D-4n | 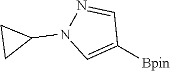 | commercially available |
| Int. D-4o | 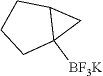 | commercially available |

### Synthesis of 7-fluoro-4-methyl-6-(4,4,5,5-tetramethyl-1,3,2-dioxaborolan-2-yl)-3,4-dihydro-2H-benzo[b][1,4]oxazine (Int D-41)

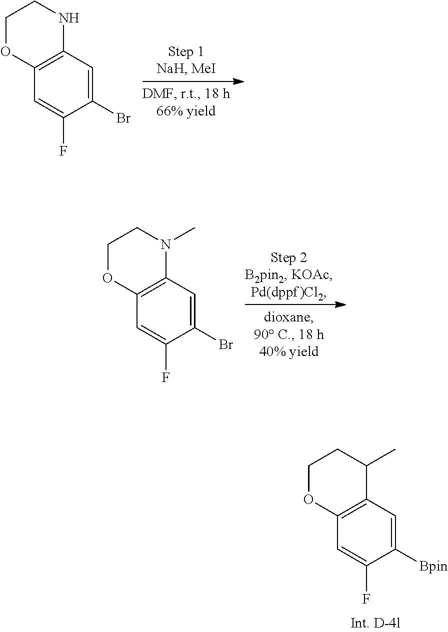

Step 1: To a solution of 6-bromo-7-fluoro-3,4-dihydro-2H-1,4-benzoxazine (600 mg, 2.59 mmol, 1.0 equiv.) in DMF (6 mL) was added NaH (155 mg, 3.88 mmol, 1.5 equiv.) at 0° C. The reaction mixture was stirred at room temperature for 20 minutes and then, Mel (0.32 mL, 5.17 mmol, 2.0 equiv.) was added. The reaction mixture was stirred at room temperature overnight. The reaction mixture was partitioned between EtOAc and water. The phases were separated, and the aqueous phase was extracted with EtOAc (2×). The combined organic layer was washed with water (3×), brine (2×), dried over sodium sulfate, filtered, and concentrated. The crude product was purified by silica gel column chromatography (0 to 40% EtOAc in heptane) to afford the product 6-bromo-7-fluoro-4-methyl-2,3-dihydro-1,4-benzoxazine (420 mg, 66% yield) as light-yellow solid.

Step 2: In a sealed tube, 6-bromo-7-fluoro-4-methyl-2,3-dihydro-1,4-benzoxazine (200 mg, 0.81 mmol), B₂pin₂ (310 mg, 1.22 mmol) and KOAc (239 mg, 2.44 mmol) were mixed under N₂. 1,4-dioxane (4 mL) was added and N₂ was bubbled through the solution for 10 minutes. Then, Pd(dppf)Cl₂·CH₂Cl₂ (66 mg, 0.08 mmol) was added and N₂ was bubbled for 5 minutes. The tube sealed and heated at 90° C. overnight. The reaction mixture was cooled down to room temperature. The reaction mixture was filtered over celite, washed with EtOAc and the filtrate evaporated. The crude product was purified by normal phase chromatography (0% to 30% EtOAc in heptane) to afford 7-fluoro-4-methyl-6-(4,4,5,5-tetramethyl-1,3,2-dioxaborolan-2-yl)-3,4-dihydro-2H-benzo[b][1,4]oxazine Int D-41 (120 mg, 40% corrected yield) as light yellow semi-solid.

LCMS: [M+H]⁺=294.2.

TABLE 19
List of Int. D-4 compounds generated by General Procedure TBM-D: Step 2

| Starting | | | | | |
|---|---|---|---|---|---|
| Material | | Product | | | |
| Int. | Int. | Int. | | | |
| D-3 | D-4 | D-5 | Structure | Characterization | Notes |
| Int D-3a | Int. D-4a | Int. D-5a | 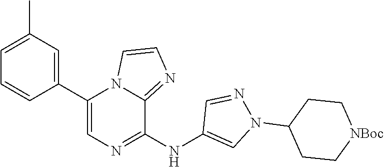 [SMILES: Cc1cccc(-c2cnc(Nc3cnn(C4CCN(C(=O)OC(C)(C)C)CC4)c3)c3nccn23)c1] | ND | Conditions: Xphos-Pd-G3, Xphos, Na₂CO₃ dioxane/water, 4 h, 90° C., microwave; Int. D-5a was directly deprotected to TBM-1 |
| Int. D-3a | Int. D-4b | Int. D-5b | 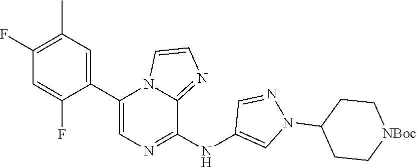 [SMILES: Cc1cc(-c2cnc(Nc3cnn(C4CCN(C(=O)OC(C)(C)C)CC4)c3)c3nccn23)c(F)cc1F] | 88% yield, light orange solid [M + H]⁺ = 510.2 ¹H NMR (400 MHz, DMSO-d₆) δ ppm 1.42 (s, 9 H), 1.71-1.84 (m, 2 H), 1.97-2.04 (m, 2 H), 2.29 (s, 3 H), 3.15-3.19 (m, 1 H), 3.98-4.14 (m, 3 H), 4.31-4.41 (m, 1 H), 7.41- 7.48 (m, 2 H), 7.58- 7.63 (m, J = 5.6 Hz, 3 H), 7.81 (s, 1 H), 8.25 (s, 1 H), 9.99 (s, 1 H). | Conditions: Pd(PPh₃)₄, NaHCO₃, dioxane/water, 90° C., 18 h |
| Int D-3b | Int. D-4b | Int. D-5c | 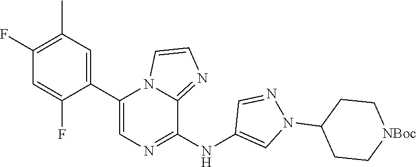 [SMILES: Cc1cc(-c2cnc(Nc3cnn(C4CCN(C(=O)OC(C)(C)C)CC4)c3)c3nccn23)c(F)cc1F] | ND | Conditions: Pd(PPh₃)₄, NaHCO₃, dioxane/water, 90° C., 18 h Int. D-5c was directly deprotected to TBM-3 |
| Int. D-3a | Int. D-4c | Int. D-5d | 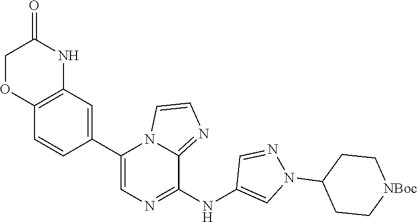 [SMILES: CC(C)(C)OC(=O)N1CCC(n2cc(Nc3ncc(-c4ccc5c(c4)NC(=O)CO5)n4ccnc34)cn2)CC1] | 86% yield, tan solid [M + H]⁺ = 531.4 ¹H NMR (400 MHz, DMSO-d₆) δ ppm 1.42 (s, 9 H), 1.77 (td, J = 12.1, 8.3 Hz, 2 H), 1.99 (s, 4 H), 2.82-2.98 (m, 2 H), 4.31- 4.41 (m, 1 H), 4.67 (s, 2 H), 7.11-7.18 (m, 2 H), 7.52- 7.69 (m, 4 H), 7.80 (s, 1 H), 7.86 (d, J = 1.0 Hz, 1 H), 8.25 (s, 1 H), 9.87-9.95 (m, 1 H), 10.84 (s, 1 H). | Conditions: Pd(dppf)Cl₂, Cs₂CO₃, dioxane/water, 90° C., overnight |
| Int. D-3c | Int. D-4b | Int. D-5e | 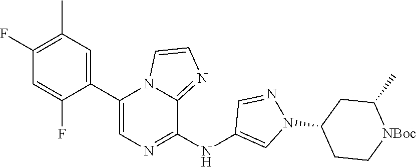 [SMILES: Cc1cc(-c2cnc(Nc3cnn([C@H]4CCN(C(=O)OC(C)(C)C)[C@@H](C)C4)c3)c3nccn23)c(F)cc1F] | 80% yield, light- orange oil [M + H]⁺ = 524.4 | Conditions: Pd(PPh₃)₄, NaHCO₃, dioxane/water, 90° C., 18 h |
| Int. D-3d | Int. D-4b | Int. D-5f | 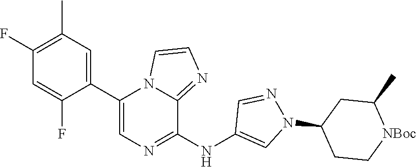 [SMILES: Cc1cc(-c2cnc(Nc3cnn([C@@H]4CCN(C(=O)OC(C)(C)C)[C@H](C)C4)c3)c3nccn23)c(F)cc1F] | ND | Conditions: Pd(PPh₃)₄, NaHCO₃, dioxane/water, 90 °C, 18 h |
| Int. D-3a | Int. D-4d | Int. D-5g | 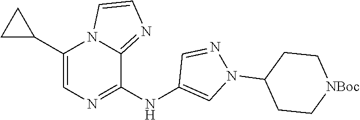 [SMILES: CC(C)(C)OC(=O)N1CCC(n2cc(Nc3ncc(C4CC4)n4ccnc34)cn2)CC1] | 33% yield, orange solid [M + H]⁺ = 424.2 ¹H NMR (400 MHz, CDCl₃) δ ppm 0.73-0.81 (m, 2 H), 1.01- 1.10 (m, 2 H), 1.49 (s, 9 H), 1.88-2.05 (m, 2 H), 2.16 (br d, J = 15.2 Hz, 2 H), 2.90 (br t, J = 12.0 Hz, 2 H), 3.49 (s, 2 H), 4.26 (tt, J = 11.3, 4.4 Hz, 2 H), 7.58 (s, 1 H), 7.61 (s, 1 H), 7.78 (s, 1 H), 7.83 (s, 1 H), 8.16 (s, 1 H). | Kumada coupling; Pd(dppf)Cl₂, THF, 60° C., overnight |
| Int. D-3a | Int. D-4e | Int. D-5h | 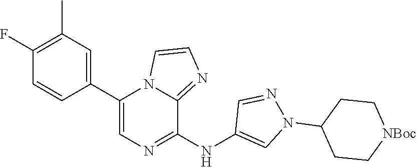 [SMILES: Cc1cc(-c2cnc(Nc3cnn(C4CCN(C(=O)OC(C)(C)C)CC4)c3)c3nccn23)ccc1F] | 64% yield, yellow solid [M + H]⁺ = 492.4 ¹H NMR (400 MHz, CDCl₃) δ ppm 1.45-1.50 (m, 9 H), 1.81- 2.01 (m, 4 H), 2.08- 2.22 (m, 4 H), 2.37 (d, J = 1.7 Hz, 3 H), 4.24 (m, 1 H), 7.18 (t, J = 8.8 Hz, 1 H), 7.29 (d, J = 1.0 Hz, 1 H), 7.42 (s, 1 H), 7.46-7.54 (m, 3 H), 7.59 (s, 1 H), 7.62 (s, 1 H), 7.66 (s, 1 H). 1ºF NMR (377 MHz, CDCl₃) δ ppm −114.65 (s, 1 F). | Conditions: Xphos-Pd-G3, Xphos, Na₂CO₃ dioxane/water, 4 h, 90° C., |
| Int. D-3a | Int. D-4f | Int. D-5i | 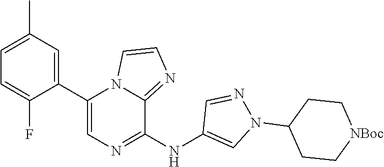 [SMILES: Cc1ccc(F)c(-c2cnc(Nc3cnn(C4CCN(C(=O)OC(C)(C)C)CC4)c3)c3nccn23)c1] | 67% yield, tan solid [M + H]⁺ = 492.2 ¹H NMR (400 MHz, DMSO-d₆) δ ppm 1.42 (s, 9 H), 1.73-1.83 (m, 2 H), 1.98-2.04 (m, 2 H), 2.37 (s, 3 H), 2.85-2.99 (m, 2 H), 4.05 (br dd, J = 12.2, 1.7 Hz, 2 H), 4.31-4.41 (m, 1 H), 7.29-7.36 (m, 1 H), 7.38-7.42 (m, 1 H), 7.43- 7.47 (m, 2 H), 7.58 (d, J = 2.0 Hz, 1 H), 7.62 (d, J = 1.0 Hz, 1 H), 7.81 (s, 1 H), 8.25 (s, 1 H), 9.98 (s, 1 H). | Conditions: Pd(PPh₃)₄, NaHCO₃, dioxane/water, 90 °C, 18 h |
| Int D-3a | Int. D-4g | Int. D-5j | 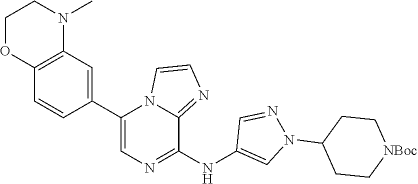 [SMILES: CN1CCOc2ccc(-c3cnc(Nc4cnn(C5CCN(C(=O)OC(C)(C)C)CC5)c4)c4nccn34)cc21] | 86% yield, tan solid [M + H]⁺ = 531.4 ¹H NMR (400 MHz, DMSO-d₆) δ ppm 1.42 (s, 9 H), 1.77 (qd, J = 12.1, 4.2 Hz, 2 H), 2.00 (br d, J = 10.5 Hz, 2 H), 2.85-2.97 (m, 5 H), 3.30 (br s, 2 H), 4.05 (br d, J = 12.0 Hz, 2 H), 4.27- 4.40 (m, 3 H), 6.81-6.88 (m, 2 H), 6.91 (d, J = 1.5 Hz, 1 H), 7.37 (s, 1 H), 7.62 (d, J = 0.7 Hz, 1 H), 7.79 (s, 1 H), 7.85-7.90 (m, 1 H), 8.25 (s, 1 H), 9.81 (s, 1 H). | Conditions: Xphos-Pd-G3, Xphos, Na₂CO₃ dioxane/water, 4 h, 90° C., |
| Int. D-3a | Int. D-4 h | Int. D-5k | 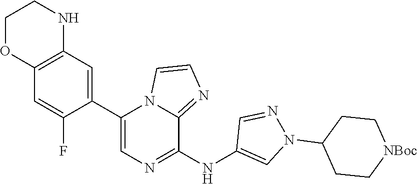 [SMILES: CC(C)(C)OC(=O)N1CCC(n2cc(Nc3ncc(-c4cc5c(cc4F)OCCN5)n4ccnc34)cn2)CC1] | 89% yield, tan solid [M + H]⁺ = 535.2 ¹H NMR (400 MHz, DMSO-d₆) δ ppm 1.42 (s, 9 H), 1.77 (qd, J = 12.1, 4.3 Hz, 2 H), 2.00 (br dd, J = 12.3, 2.3 Hz, 2 H), 2.84- 2.98 (m, 2 H), 3.29 (s, 2 H), 4.05 (br d, J = 11.7 Hz, 2 H), 4.22 (t, J = 4.2 Hz, 2 H), 4.35 (tt, J = 11.4, 4.0 Hz, 1 H), 5.89 (s, 1 H), 6.71 (d, J = 7.3 Hz, 1 H), 6.77 (d, J = 10.8 Hz, 1 H), 7.33 (s, 1 H), 7.55 (dd, J = 2.9, 1.2 Hz, 1 H), 7.60 (d, J = 1.2 Hz, 1 H), 7.75-7.84 (m, 1 H), 8.24 (s, 1 H), 9.89 (s, 1 H). 9F NMR (377 MHz, DMSO-d₆) δ ppm −126.03- −125.74 (m, 1 F). | Conditions: Pd(PPh₃)₄, NaHCO₃, dioxane/water, 90 °C, 18 h |
| Int. D-3e | Int. D-4b | Int. D-51′ | 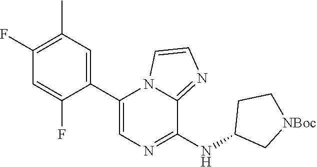 | 82% yield, orange semi-solid [M + H]⁺ = 513.2 ¹H NMR (400 MHz, DMSO-d₆) δ ppm 1.20-1.33 (m, 2 H), 1.39 (s, 9 H), 1.74-1.86 (m, 2 H), 1.86-1.99 (m, 1 H), 2.15- 2.26 (m, 1 H), 2.28 (s, 3 H), 2.29-2.37 (m, 1 H), 2.59- 2.70 (m, 2 H), 2.75- 2.91 (m, 3 H), 2.96-3.03 (m, 1 H), 3.75-3.90 (m, 2 H), 4.55-4.69 (m, 1 H), 7.31 (s, 1 H), 7.38-7.50 (m, 2 H), 7.51-7.60 (m, 3 H), 8.16 (s, 1 H). ¹⁹F NMR (377 MHz, DMSO-d₆) δ ppm −112.17 (q, J = 9.1 Hz, 1 F),- 111.44 (q, J = 8.2 Hz, 1 F). | Int. D-51′ was converted to Int. D-51 by a two-step procedure given below Conditions: Pd(PPh₃)₄, NaHCO₃, dioxane/water, 90° C., 18 h |
| Int. D-3f | Int. D-4b | Int. D-5m | 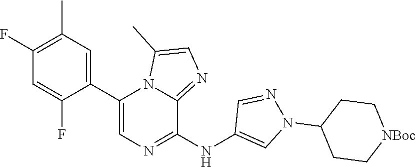 [SMILES: Cc1cc(-c2cnc(Nc3cnn(C4CCN(C(=O)OC(C)(C)C)CC4)c3)c3ncc(C)n23)c(F)cc1F] | 62% yield, yellow semi-solid [M + H]⁺ = 524.2 ¹H NMR (400 MHz, DMSO-d₆) δ ppm 1.42 (s, 9 H), 1.71-1.84 (m, 2 H), 1.90 (s, 3 H), 1.96-2.04 (m, 2 H), 2.28 (s, 3 H), 2.80-2.99 (m, 2 H), 3.98-4.12 (m, 2 H), 4.31-4.41 (m, 1 H), 7.26 (s, 1 H), 7.38-7.51 (m, 2 H), 7.56 (t, J = 8.7 Hz, 1 H), 7.83 (s, 1 H), 8.19-8.23 (m, 1 H), 10.07- 10.32 (m, 1 H). ¹⁹F NMR (377 MHz, DMSO-d₆) δ ppm −113.10- −112.88 (m, 1 F),- 110.60-−110.18 (m, 1 F). | Conditions: Pd(PPh₃)₄, NaHCO₃, dioxane/water, 90° C., 18 h |
| Int. D-3g | Int D-4b | Int. D-5n | 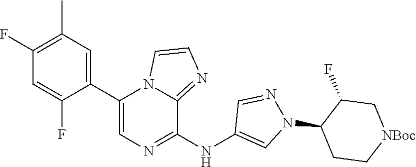 [SMILES: Cc1cc(-c2cnc(Nc3cnn([C@@H]4CCN(C(=O)OC(C)(C)C)C[C@H]4F)c3)c3nccn23)c(F)cc1F] | 66% yield, light yellow semi-solid [M + H]⁺ = 528.2. ¹H NMR (400 MHz, DMSO-d₆) δ ppm 1.43 (s, 9 H), 1.89-2.09 (m, 2 H), 2.23-2.32 (m, 3 H), 2.81-3.14 (m, 2 H), 3.98 (br d, J = 13.0 Hz, 1 H), 4.28 (br s, 1 H), 4.48-4.64 (m, 1 H), 4.68-4.91 (m, 1 H), 7.36-7.50 (m, 2 H), 7.56- 7.68 (m, 3 H), 7.88 (s, 1 H), 8.34 (s, 1 H), 10.05 (s, 1 H). ¹⁹F NMR (377 MHz, DMSO-d₆) δ ppm −186.93- −186.56 (m, 1 F),- 112.18 (q, J = 8.6 Hz, 1 F),-111.21 (q, J = 8.2 Hz, 1 F). | Conditions: Pd(PPh₃)₄, NaHCO₃, dioxane/water, 90° C., 18 h |
| Int D-3h | Int. D-4b | Int. D-50 | 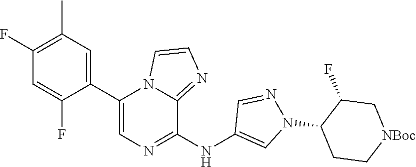 [SMILES: Cc1cc(-c2cnc(Nc3cnn([C@H]4CCN(C(=O)OC(C)(C)C)C[C@H]4F)c3)c3nccn23)c(F)cc1F] | 72% yield, brown solid [M + H]⁺ = 528.2 ¹H NMR (400 MHz, DMSO-d₆) δ ppm 1.42 (s, 9 H), 1.92-2.03 (m, 1 H), 2.11-2.24 (m, 1 H), 2.29 (s, 3 H), 2.87-3.11 (m, 1 H), 3.12-3.22 (m, 1 H), 4.10-4.22 (m, 1 H), 4.22- 4.40 (m, 1 H), 4.53- 4.68 (m, 1 H), 4.93-5.16 (m, 1 H), 7.41-7.49 (m, 2 H), 7.56-7.67 (m, 3 H), 7.86 (s, 1 H), 8.26 (s, 1 H), 10.03 (s, 1 H). ¹⁹F NMR (377 MHz, DMSO-d₆) δ ppm −202.23- −201.62 (m, 1 F),- 112.14 (q, J = 8.2 Hz, 1 F),-111.23 (q, J = 9.1 Hz, 1 F). | Conditions: Pd(PPh₃)₄, NaHCO₃, dioxane/water, 90° C., 18 h |
| Int D-3i | Int. D-4b | Int. D-5p | 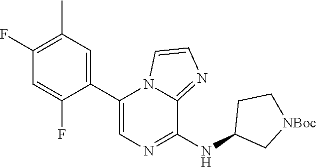 | 79% yield, orange semi-solid [M + H]⁺ = 513.2 ¹H NMR (400 MHz, DMSO-d₆) δ ppm 1.21-1.34 (m, 2 H), 1.39 (s, 9 H), 1.75-1.85 (m, 2 H), 1.88-1.98 (m, 1 H), 2.15- 2.37 (m, 5 H), 2.59- 2.71 (m, 2 H), 2.74-2.89 (m, 3 H), 2.97-3.04 (m, 1 H), 3.83 (br d, J = 12.0 Hz, 2 H), 4.57- 4.67 (m, 1 H), 7.31 (s, 1 H), 7.43 (t, J = 9.9 Hz, 1 H), 7.49 (d, J = 7.1 Hz, 1 H), 7.52-7.59 (m, 3 H), 8.16 (s, 1 H) | Int. D-5p′ was converted to Int. D-5p by a two-step procedure given below Conditions: Pd(PPh₃)₄, NaHCO₃, dioxane/water, 90° C., 18 h |
| Int D-3a | Int. D-4i | Int. D-5q | 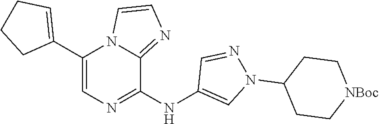 [SMILES: CC(C)(C)OC(=O)N1CCC(n2cc(Nc3ncc(C4=CCCC4)n4ccnc34)cn2)CC1] | 53% yield, light yellow solid [M + H]⁺ = 450.2 ¹H NMR (400 MHz, DMSO-d₆) δ ppm 1.42 (s, 9 H), 1.73-1.82 (m, 4 H), 1.94-2.03 (m, 4 H), 2.59-2.65 (m, 2 H), 2.81 (br t, J = 6.7 Hz, 2 H), 3.99-4.14 (m, 2 H), 4.30-4.39 (m, 1 H), 6.47 (br s, 1 H), 7.47 (s, 1 H), 7.69 (d, J = 1.0 Hz, 1 H), 7.78 (s, 1 H), 8.23 (s, 1 H), 8.28 (d, J = 1.0 Hz, 1 H), 9.91 (s, 1 H). | Conditions: Pd₂(dba)₃, Cs₂CO₃, Pcy3, dioxane/water, 90 °C, 24 h |
| Int. D-3j | Int. D-4b | Int. D-5r | 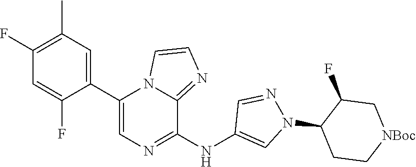 [SMILES: Cc1cc(-c2cnc(Nc3cnn([C@@H]4CCN(C(=O)OC(C)(C)C)C[C@@H]4F)c3)c3nccn23)c(F)cc1F] | 83% yield, brown oil [M + H]⁺ = 528.2 ¹H NMR (400 MHz, DMSO-d₆) δ ppm 1.12-1.31 (m, 1 H), 1.42 (s, 9 H), 1.94-2.04 (m, 1 H), 2.10-2.24 (m, 1 H), 2.29 (s, 3 H), 2.86-3.10 (m, 1 H), 4.10-4.22 (m, 1 H), 4.24- 4.40 (m, 1 H), 4.53- 4.73 (m, 1 H), 4.86-5.12 (m, 1 H), 7.37-7.49 (m, 2 H), 7.55-7.67 (m, 3 H), 7.86 (s, 1 H), 8.26 (s, 1 H), 10.03 (s, 1 H). ¹⁹F NMR (377 MHz, DMSO-d₆) δ ppm −112.14 (q, J = 7.7 Hz, 1 F),- 111.23 (q, J = 7.7 Hz, 1 F). One aliphatic F not observed. | Conditions: Pd(PPh₃)₄, NaHCO₃, dioxane/water, 90° C., 18 h |
| Int. D-3k | Int. D-4b | Int D-5s | 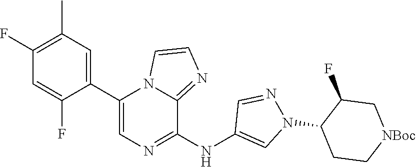 [SMILES: Cc1cc(-c2cnc(Nc3cnn([C@H]4CCN(C(=O)OC(C)(C)C)C[C@@H]4F)c3)c3nccn23)c(F)cc1F] | 68% yield, brown foam [M + H]⁺ = 528.2. ¹H NMR (400 MHz, DMSO-d₆) δ ppm 1.24-1.31 (m, 1 H), 1.43 (s, 9 H), 1.90-2.05 (m, 2 H), 2.29 (s, 3 H), 3.98 (br d, J = 11.7 Hz, 1 H), 4.19- 4.35 (m, 1 H), 4.46- 4.63 (m, 1 H), 4.66-4.76 (m, 1 H), 4.77-4.90 (m, 1 H), 7.41-7.47 (m, 2 H), 7.60- 7.65 (m, 3 H), 7.87 (s, 1 H), 8.33 (s, 1 H), 10.04 (s, 1 H). ¹⁹F NMR (377 MHz, DMSO-d₆) δ ppm −187.18- −186.32 (m, 1 F),- 112.18 (q, J = 8.2 Hz, 1 F),-111.21 (q, J = 8.2 Hz, 1 F). | Conditions: Pd(PPh₃)₄, NaHCO₃, dioxane/water, 90° C., 18 h |
| Int. D-3a | Int. D-4j | Int. D-5t | 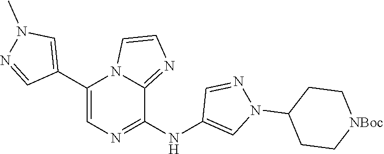 [SMILES: Cn1cc(-c2cnc(Nc3cnn(C4CCN(C(=O)OC(C)(C)C)CC4)c3)c3nccn23)cn1] | 79% yield, orange oil [M + H]⁺ = 464.2 ¹H NMR (400 MHz, DMSO-d₆) δ ppm 1.42 (s, 9 H), 1.76 (qd, J = 12.1, 4.1 Hz, 2 H), 1.96- 2.05 (m, 2 H), 2.79- 3.02 (m, 2 H), 3.94 (s, 3 H), 4.05 (br d, J = 12.2 Hz, 2 H), 4.28-4.42 (m, 1 H), 7.53 (s, 1 H), 7.68 (d, J = 0.9 Hz, 1 H), 7.78 (s, 1 H), 7.93 (s, 1 H), 8.04 (d, J = 1.0 Hz, 1 H), 8.13 (3H, s), 8.23 (s, 1 H), 8.30 (s, 1 H), 9.80 (s, 1 H), 12.76 (br s, 3 H). | Conditions: Xphos-Pd-G3, Xphos, Na₂CO₃ dioxane/water, 4 h, 90° C., |
| Int. D-3a | Int D-4k | Int. D-5u | 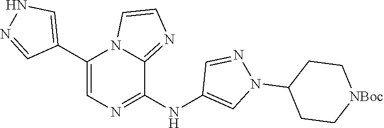 [SMILES: CC(C)(C)OC(=O)N1CCC(n2cc(Nc3ncc(-c4cn[nH]c4)n4ccnc34)cn2)CC1] | 55% yield, light- yellow semi-solid [M + H]⁺ = 450.2 ¹H NMR (400 MHz, DMSO-d₆) δ ppm 1.42 (s, 9 H), 1.69-1.84 (m, 2 H), 1.95-2.05 (m, 2 H), 2.81-3.01 (m, 2 H), 3.98- 4.10 (m, 2 H), 4.29- 4.40 (m, 1 H), 7.56 (s, 1 H), 7.65- 7.69 (m, 1 H), 7.77- 7.81 (m, 1 H), 7.95-8.11 (m, 2 H), 8.20-8.25 (m, 1 H), 8.35 (br s, 1 H), 9.79 (s, 1 H), 13.28 (br s, 1 H). | Conditions: Xphos-Pd-G3, Xphos, Na₂CO₃ dioxane/water, 4 h, 90° C., |
| Int. D-3a | Int. D-4l | Int. D-5v | 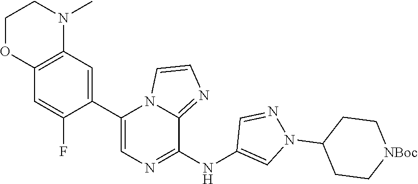 [SMILES: CN1CCOc2cc(F)c(-c3cnc(Nc4cnn(C5CCN(C(=O)OC(C)(C)C)CC5)c4)c4nccn34)cc21] | 62% yield, tan solid [M + H]⁺ = 549.2 | Conditions: Pd(PPh₃)₄, NaHCO₃, dioxane/water, 90° C., 18 h |
| Int. D-3m | Int. D-4b | Int. D-5w | 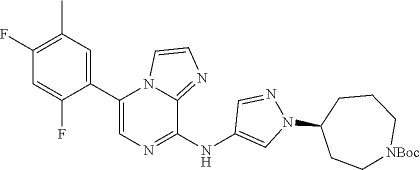 [SMILES: Cc1cc(-c2cnc(Nc3cnn([C@H]4CCCN(C(=O)OC(C)(C)C)CC4)c3)c3nccn23)c(F)cc1F] | 69% yield, brown solid [M + H]⁺ = 524.2 ¹H NMR (400 MHz, DMSO-d₆) δ ppm 1.41-1.47 (m, 9 H), 1.61- 1.76 (m, 1 H), 1.81- 2.07 (m, 4 H), 2.09-2.13 (m, 1 H), 2.30 (s, 3 H), 3.21-3.31 (m, 1 H), 3.37-3.48 (m, 2 H), 3.53-3.69 (m, 1 H), 4.26- 4.37 (m, 1 H), 7.45 (d, J = 10.0 Hz, 2 H), 7.58-7.65 (m, 3 H), 7.80 (s, 1 H), 8.21 (s, 1 H), 10.00 (s, 1 H). ¹⁹F NMR (377 MHz, DMSO-d₆) δ ppm −112.18 (q, J = 8.6 Hz, 1 F),- 111.02-−110.88 (m, 1 F). | Conditions: Pd(PPh₃)₄, NaHCO₃, dioxane/water, 90° C., 18 h absolute stereochemistry not determined arbitrarily assigned |
| Int. D-32 | Int. D-4b | Int. D-5x | 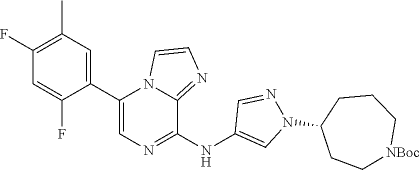 [SMILES: Cc1cc(-c2cnc(Nc3cnn([C@@H]4CCCN(C(=O)OC(C)(C)C)CC4)c3)c3nccn23)c(F)cc1F] | 81% yield, brown solid [M + H]⁺ = 524.2 ¹H NMR (400 MHz, DMSO-d₆) δ ppm 1.45 (s, 9 H), 1.67-1.78 (m, 2 H), 1.83-1.95 (m, 3 H), 2.29-2.34 (m, 3 H), 3.23- 3.31 (m, 2 H), 3.41 (br dd, J = 7.0, 5.3 Hz, 2 H), 3.57- 3.66 (m, 1 H), 4.28- 4.37 (m, 1 H), 7.43-7.52 (m, 2 H), 7.60-7.67 (m, 3 H), 7.81 (br s, 1 H), 8.23 (s, 1 H), 10.01 (s, 1 H). ¹⁹F NMR (377 MHz, DMSO-d₆) δ ppm −112.27- −112.12 (m, 1 F),- 111.34-−111.19 (m, 1 F). | Conditions: Pd(PPh₃)₄, NaHCO₃, dioxane/water, 90 °C, 18 h absolute stereochemistry not determined arbitrarily assigned |
| Int. D-3a | Int. D-4m | Int. D-5y | 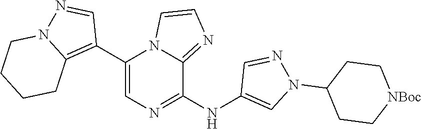 [SMILES: CC(C)(C)OC(=O)N1CCC(n2cc(Nc3ncc(-c4cnn5c4CCCC5)n4ccnc34)cn2)CC1] | 61% yield, light- yellow solid [M + H]⁺ = 504.2 ¹H NMR (400 MHz, DMSO-d₆) δ ppm 1.42 (s, 9 H), 1.70-1.87 (m, 4 H), 1.94-2.08 (m, 4 H), 2.75 (br t, J = 6.1 Hz, 2 H), 2.81- 3.00 (m, 2 H), 4.05- 4.12 (m, 2 H), 4.16 (br t, J = 5.9 Hz, 2 H), 4.28- 4.41 (m, 1 H), 7.30 (s, 1 H), 7.62 (s, 1 H), 7.74-7.83 (m, 3 H), 8.24 (s, 1 H), 9.78 (s, 1 H). | Conditions: Xphos-Pd-G3, Xphos, Na₂CO₃ dioxane/water, 4 h, 90° C., |
| Int. D-3a | Int. D-4n | Int. D-5z | 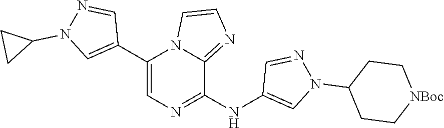 [SMILES: CC(C)(C)OC(=O)N1CCC(n2cc(Nc3ncc(-c4cnn(C5CC5)c4)n4ccnc34)cn2)CC1] | 41% yield, light orange solid [M + H]⁺ = 490.2 ¹H NMR (400 MHz, DMSO-d₆) δ ppm 0.99-1.05 (m, 2 H), 1.13- 1.18 (m, 2 H), 1.42 (s, 9 H), 1.70-1.83 (m, 2 H), 1.96- 2.03 (m, 2 H), 2.83- 2.98 (m, 2 H), 3.82 (tt, J = 7.4, 3.9 Hz, 1 H), 3.99- 4.10 (m, 2 H), 4.31- 4.39 (m, 1 H), 7.54 (s, 1 H), 7.68 (d, J = 1.0 Hz, 1 H), 7.79 (s, 1 H), 7.93 (s, 1 H), 8.08 (d, J = 1.2 Hz, 1 H), 8.22 (s, 1 H), 8.37 (s, 1 H), 9.80 (s, 1 H). | Conditions: Xphos-Pd-G3, Xphos, Na₂CO₃ dioxane/water, 4 h, 90° C., |
| Int. D-3a | Int. D-4o | Int. D-5aa | 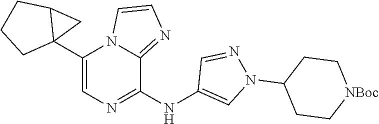 [SMILES: CC(C)(C)OC(=O)N1CCC(n2cc(Nc3ncc(C45CCCC4C5)n4ccnc34)cn2)CC1] | yellow semi-solid 71% yield, light [M + H]⁺ = 464.3 | Conditions: Pd(PPh₃)₄, NaHCO₃, dioxane/water, 90° C., 18 h |

ND = not determined

### Synthesis of tert-butyl (R)-4-(3-((5-(2,4-difluoro-5-methylphenyl)imidazo[1,2-a]pyrazin-8-yl)amino)154yrrolidine-1-yl)piperidine-1-carboxylate (Int. D-5l) from Int. D-5l′

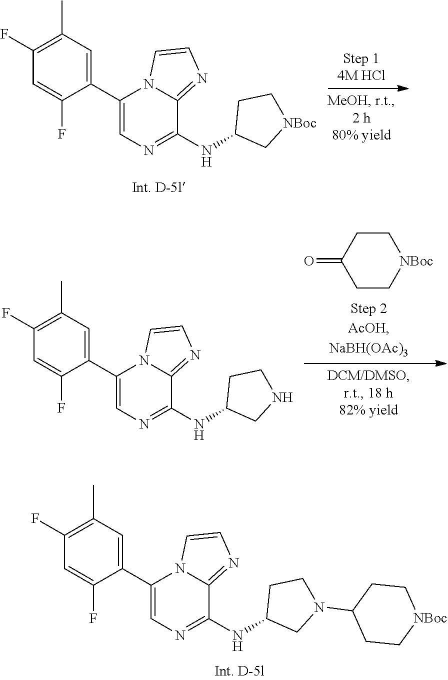 [SMILES: Cc1cc(-c2cnc(N[C@@H]3CCN(C(=O)OC(C)(C)C)C3)c3nccn23)c(F)cc1F ; Cc1cc(-c2cnc(N[C@@H]3CCNC3)c3nccn23)c(F)cc1F ; CC(C)(C)OC(=O)N1CCC(=O)CC1 ; Cc1cc(-c2cnc(N[C@@H]3CCN(C4CCN(C(=O)OC(C)(C)C)CC4)C3)c3nccn23)c(F)cc1F]

Step 1: To a solution of tert-butyl (3R)-3-[[5-(2,4-difluoro-5-methyl-phenyl)imidazo[1,2-a]pyrazin-8-yl]amino]pyrrolidine-1-carboxylate Int. D-5l′ (290 mg, 0.68 mmol, 1 eq.) in MeOH (5 mL, 0.14 M) was added 4 M HCl in 1,4-dioxane (2.53 mL, 10.1 mmol, 15 eq.) and the reaction was stirred at r.t. for 2 h. The solvent was evaporated and co-evaporated with MeOH and with MTBE to afford (R)-5-(2,4-difluoro-5-methylphenyl)-N-(154yrrolidine-3-yl)imidazo[1,2-a]pyrazin-8-amine (283 mg, 80% yield) as a brown solid as bis HCl salt.

LCMS: [M+H]⁺=330.2.

¹H NMR (400 MHz, DMSO-d₆) δ ppm 2.16-2.21 (m, 1H), 2.23-2.35 (m, 4H), 3.22-3.39 (m, 2H), 3.39-3.48 (m, 1H), 3.48-3.56 (m, 1H), 4.79-4.84 (m, 1H), 7.43-7.51 (m, 2H), 7.54-7.61 (m, 1H), 7.71-7.79 (m, 2H), 8.97-9.10 (m, 1H), 9.20-9.33 (m, 1H).

Step 2: To (R)-5-(2,4-difluoro-5-methylphenyl)-N-(154yrrolidine-3-yl)imidazo[1,2-a]pyrazin-8-amine (280 mg, 0.77 mmol, 1 eq.) in DMSO (2.56 mL, 0.3 M) at r.t. was added DIPEA (0.67 mL, 3.8 mmol, 5 eq.) followed by tert-butyl 4-oxopiperidine-1-carboxylate (152.51 mg, 0.77 mmol, 1 eq.) and AcOH (0.03 mL, 0.54 mmol, 0.7 eq.) under N₂ and the solution was stirred for 10-15 min. Then, NaBH(Oac)₃ (324 mg, 1.53 mmol, 2 eq.) was added and the reaction mixture was stirred at r.t, overnight. The solvent was evaporated and the reaction mixture was purified by reverse phase column chromatography (5% to 100% MeOH in water w/ 0.1% formic acid). The fractions were combined and concentrated to afford tert-butyl (R)-4-(3-((5-(2,4-difluoro-5-methylphenyl)imidazo[1,2-a]pyrazin-8-yl)amino)pyrrolidin-1-yl)piperidine-1-carboxylate Int. D-51 (320 mg, 82% yield) as an orange semi-solid as a formic acid salt.

LCMS: [M+H]⁺=513.2.

¹H NMR (400 MHz, DMSO-d₆) δ ppm 1.20-1.33 (m, 2H), 1.39 (s, 9H), 1.74-1.86 (m, 2H), 1.86-1.99 (m, 1H), 2.15-2.26 (m, 1H), 2.28 (s, 3H), 2.29-2.37 (m, 1H), 2.59-2.70 (m, 2H), 2.75-2.91 (m, 3H), 2.96-3.03 (m, 1H), 3.75-3.90 (m, 2H), 4.55-4.69 (m, 1H), 7.31 (s, 1H), 7.38-7.50 (m, 2H), 7.51-7.60 (m, 3H), 8.16 (s, 1H).

¹⁹F NMR (377 MHz, DMSO-d₆) δ ppm −112.17 (q, J=9.1 Hz, 1 F), −111.44 (q, J=8.2 Hz, 1 F).

### Synthesis of tert-butyl (S)-4-(3-((5-(2,4-difluoro-5-methylphenyl)imidazo[1,2-a]pyrazin-8-yl)amino)pyrrolidin-1-yl)piperidine-1-carboxylate (Int. D-5p) from Int. D-5p′

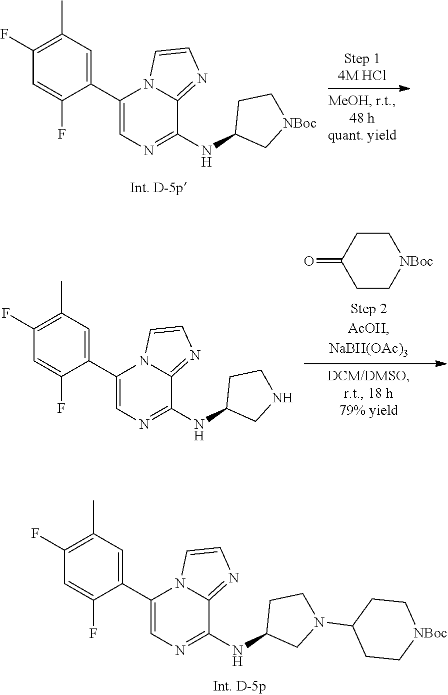 [SMILES: Cc1cc(-c2cnc(N[C@H]3CCN(C(=O)OC(C)(C)C)C3)c3nccn23)c(F)cc1F ; Cc1cc(-c2cnc(N[C@H]3CCNC3)c3nccn23)c(F)cc1F ; CC(C)(C)OC(=O)N1CCC(=O)CC1 ; Cc1cc(-c2cnc(N[C@H]3CCN(C4CCN(C(=O)OC(C)(C)C)CC4)C3)c3nccn23)c(F)cc1F]

Step 1: To a solution of tert-butyl (S)-3-((5-(2,4-difluoro-5-methylphenyl)imidazo[1,2-a]pyrazin-8-yl)amino)pyrrolidine-1-carboxylate (273 mg, 0.64 mmol, 1 equiv.) in MeOH (5 mL, 0.13 M) was added 4 M HCl in 1,4-dioxane (2.38 mL, 9.54 mmol, 15 equiv.) and the reaction was stirred at r.t for 48 h. The solvent was evaporated and co-evaporated with MeOH and with MTBE to afford (S)-5-(2,4-difluoro-5-methylphenyl)-N-(pyrrolidin-3-yl)imidazo[1,2-a]pyrazin-8-amine (310 mg, quantitative) as a brown solid as a bis HCl salt.

LCMS: [M+H]⁺=330.2.

¹H NMR (400 MHz, DMSO-d₆) δ ppm 2.13-2.23 (m, 1H), 2.24-2.37 (m, 4H), 3.24-3.48 (m, 3H), 3.51-3.61 (m, 1H), 4.76-4.89 (m, 1H), 7.43-7.54 (m, 2H), 7.55-7.62 (m, 1H), 7.82 (br s, 1H), 7.88 (br s, 1H), 8.95-9.34 (m, 2H), 9.49 (br s, 1H).

Step 2: To (S)-5-(2,4-difluoro-5-methylphenyl)-N-(pyrrolidin-3-yl)imidazo[1,2-a]pyrazin-8-amine (308 mg, 0.74 mmol, 1 equiv.) in CH₂Cl₂ (3 mL, 0.2 M) and DMSO (0.50 mL, 0.2 M) at r.t. was added DIPEA (0.65 mL, 3.7 mmol, 5 equiv.) followed by tert-butyl 4-oxopiperidine-1-carboxylate (148 mg, 0.74 mmol, 1 equiv.) and AcOH (0.04 mL, 0.74 mmol, 1 equiv.) under N₂ and the solution was stirred for 10-15 min. Then, NaBH(OAc)₃ (316 mg, 1.49 mmol, 2 equiv.) was added and the reaction mixture was stirred at r.t., overnight. The solvent was evaporated, and the reaction mixture was purified by reverse phase column chromatography (5% to 100% MeOH in water w/ 0.1% formic acid). The fractions were combined and concentrated to afford tert-butyl (S)-4-(3-((5-(2,4-difluoro-5-methylphenyl)imidazo[1,2-a]pyrazin-8-yl)amino)pyrrolidin-1-yl)piperidine-1-carboxylate Int. D-5p (344 mg, 79% yield) as an orange semi-solid as a formic acid salt.

LCMS: [M+H]⁺=513.2.

¹H NMR (400 MHz, DMSO-d₆) δ ppm 1.21-1.34 (m, 2H), 1.39 (s, 9H), 1.75-1.85 (m, 2H), 1.88-1.98 (m, 1H), 2.15-2.37 (m, 5H), 2.59-2.71 (m, 2H), 2.74-2.89 (m, 3H), 2.97-3.04 (m, 1H), 3.83 (br d, J=12.0 Hz, 2H), 4.57-4.67 (m, 1H), 7.31 (s, 1H), 7.43 (t, J=9.9 Hz, 1H), 7.49 (d, J=7.1 Hz, 1H), 7.52-7.59 (m, 3H), 8.16 (s, 1H).

### Synthesis of TBM-D: Step 3

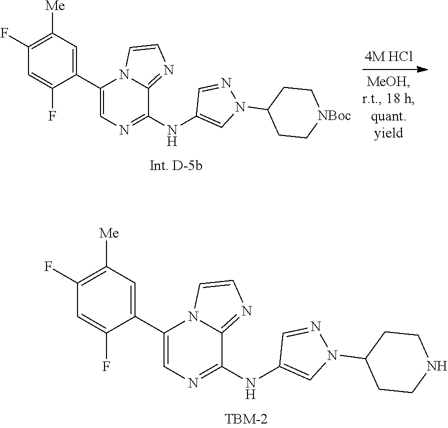 [SMILES: Cc1cc(-c2cnc(Nc3cnn(C4CCN(C(=O)OC(C)(C)C)CC4)c3)c3nccn23)c(F)cc1F ; Cc1cc(-c2cnc(Nc3cnn(C4CCNCC4)c3)c3nccn23)c(F)cc1F]

To a solution of tert-butyl 4-(4-((5-(2,4-difluoro-5-methylphenyl)imidazo[1,2-a]pyrazin-8-yl)amino)-1H-pyrazol-1-yl)piperidine-1-carboxylate Int. D-5b (2.65 g, 5.2 mmol, 1 equiv.) in MeOH (14 mL, 0.37 M) was added 4 M HCl in 1,4-dioxane (19.5 mL, 78 mmol, 15 equiv.). The reaction mixture was stirred at room temperature overnight. The reaction mixture was concentrated under reduced pressure and the residue was co-evaporated with MeOH (2×) and MTBE (2×) to give 5-(2,4-difluoro-5-methylphenyl)-N-(1-(piperidin-4-yl)-1H-pyrazol-4-yl)imidazo[1,2-a]pyrazin-8-amine TBM-2 (2.62 g, quantitative yield) as a tan solid as a bis HCl salt.

LCMS: [M+H]⁺=410.2.

¹H NMR (400 MHz, DMSO-d₆) δ ppm 2.16-2.24 (m, 4H), 2.27-2.31 (m, 3H), 2.99-3.12 (m, 2H), 3.34-3.43 (m, 2H), 4.49-4.59 (m, 1H), 7.49 (br t, J=9.9 Hz, 1H), 7.57-7.63 (m, 2H), 7.81 (s, 1H), 7.86 (br s, 1H), 7.96 (s, 1H), 8.23 (s, 1H), 8.95-9.09 (m, 1H), 9.19-9.32 (m, 1H), 10.77-10.90 (m, 1H).

¹⁹F NMR (377 MHz, DMSO-d₆) δ ppm −111.93 (s, 1 F), −110.28 (s, 1 F).

TABLE 20
List of TBM-D compounds generated by General Procedure TBM-D: Step 3

| Starting | | | |
|---|---|---|---|
| Material. | Product | | |
| Int. D-5 | TBM# | Structure | Characterization |
| Int. D-5a | TBM-1 | 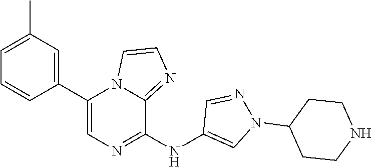 [SMILES: Cc1cccc(-c2cnc(Nc3cnn(C4CCNCC4)c3)c3nccn23)c1] | 88% yield, light-yellow solid [M + H]⁺ = 374.5 ¹H NMR (400 MHz, methanol-d₄) δ ppm 2.27-2.41 (m, 4 H), 2.45 (s, 3 H), 3.19- 3.29 (m, 2 H), 3.59 (br d, J = 13.2 Hz, 2 H), 4.60 (tt, J = 10.1, 4.8 Hz, 1 H), 7.31 (s, 1 H), 7.39-7.51 (m, 4 H), 7.81 (s, 2 H), 7.94 (s, 1 H), 8.24 (s, 1 H). |
| Int. D-5b | TBM-2 | 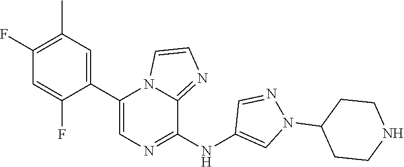 [SMILES: Cc1cc(-c2cnc(Nc3cnn(C4CCNCC4)c3)c3nccn23)c(F)cc1F] | quant. yield, tan solid [M + H]⁺ = 410.2 ¹H NMR (400 MHz, DMSO-d₆) δ ppm 2.16- 2.24 (m, 4 H), 2.27-2.31 (m, 3 H), 2.99- 3.12 (m, 2 H), 3.34-3.43 (m, 2 H), 4.49- 4.59 (m, 1 H), 7.49 (br t, J = 9.9 Hz, 1 H), 7.57-7.63 (m, 2 H), 7.81 (s, 1 H), 7.86 (br s, 1 H), 7.96 (s, 1 H), 8.23 (s, 1 H), 8.95- 9.09 (m, 1 H), 9.19-9.32 (m, 1 H), 10.77- 10.90 (m, 1 H). ¹⁹F NMR (377 MHz, DMSO-d₆) δ ppm −111.93 (s, 1 F), −110.28 (s, 1 F). |
| Int. D-5c | TBM-3 | 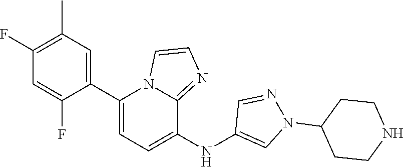 [SMILES: Cc1cc(-c2ccc(Nc3cnn(C4CCNCC4)c3)c3nccn23)c(F)cc1F] | 99% yield, light yellow solid [M + H]⁺ = 409.2; [M + 2H]²⁺ = 205.4 ¹H NMR (400 MHz, DMSO-d₆) δ ppm 2.13- 2.27 (m, 4 H) 2.30 (s, 3 H) 3.08 (m, J = 9.54 Hz, 2 H) 3.40 (d, J = 12.96 Hz, 2 H) 4.53 (dt, J = 10.09, 4.86 Hz, 1 H) 7.10 (d, J = 8.31 Hz, 1 H) 7.31 (d, J = 8.31 Hz, 2 H) 7.52 (t, J = 10.03 Hz, 1 H) 7.60 (t, J = 8.44 Hz, 1 H) 7.67 (s, 2 H) 7.97-8.01 (m, 1 H) 8.03 (s, 1 H) 8.33 (d, J = 2.20 Hz, 1 H) 8.98 (s, 1 H) 9.19-9.30 (m, 1 H) 9.49 (s, 1 H) ¹⁹F NMR (377 MHz, DMSO-d₆) δ ppm −112.39 (q, J = 8.63 Hz, 1 F)-109.95 (q, J = 9.08 Hz, 1 F) |
| Int. D-5d | TBM-4 | 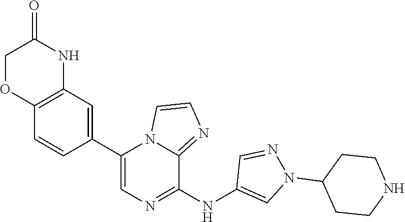 [SMILES: O=C1COc2ccc(-c3cnc(Nc4cnn(C5CCNCC5)c4)c4nccn34)cc2N1] | quant. yield, brown solid [M + H]⁺ = 431.2 ¹H NMR (400 MHz, DMSO-d₆) δ ppm 2.11- 2.25 (m, 3 H), 2.99-3.13 (m, 2 H), 3.40 (br d, J = 12.5 Hz, 2 H), 4.51 (dt, J = 10.0, 5.2 Hz, 2 H), 4.68 (s, 2 H), 7.08-7.31 (m, 3 H), 7.38-7.52 (m, 1 H), 7.76-7.90 (m, 2 H), 7.96 (d, J = 0.7 Hz, 1 H), 8.24 (s, 1 H), 8.70-8.86 (m, 1 H), 8.97-9.13 (m, 1 H), 10.41-10.53 (m, 1 H), 10.91 (s, 1 H). |
| Int. D-5e | TBM-5 | 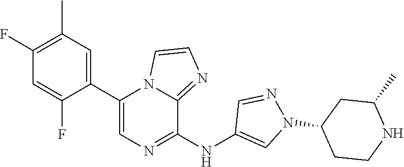 [SMILES: Cc1cc(-c2cnc(Nc3cnn([C@H]4CCN[C@@H](C)C4)c3)c3nccn23)c(F)cc1F] | quant. yield, yellow solid [M + H]⁺ = 424.4 |
| Int. D-5f | TBM-6 | 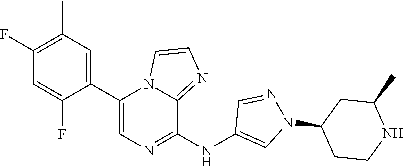 [SMILES: Cc1cc(-c2cnc(Nc3cnn([C@@H]4CCN[C@H](C)C4)c3)c3nccn23)c(F)cc1F] | 83% yield, orange solid [M + H]⁺ = 424.4 ¹H NMR (400 MHz, DMSO-d₆) δ ppm 1.31 (d, J = 6.6 Hz, 3 H), 1.97 (q, J = 12.4 Hz, 1 H), 2.05-2.27 (m, 3 H), 3.00-3.11 (m, 1 H), 3.27-3.47 (m, 2 H), 4.50-4.60 (m, 1 H), 7.48 (t, J = 9.9 Hz, 1 H), 7.51 (s, 1 H), 7.61 (t, J = 8.3 Hz, 1 H), 7.75 (s, 1 H), 7.81 (s, 1 H), 7.83 (s, 1 H), 8.23 (s, 1 H), 8.78- 8.92 (m, 1 H), 9.33 (br d, J = 8.3 Hz, 1 H), 10.48 (br s, 1 H). ¹⁹F NMR (377 MHz, DMSO-d₆) δ ppm −112.12-−111.95 (m, 1 F), −110.78- −110.57 (m, 1 F). |
| Int. D-5g | TBM-7 | 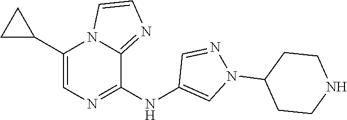 [SMILES: c1cn2c(C3CC3)cnc(Nc3cnn(C4CCNCC4)c3)c2n1] | 15% yield, light-orange solid [M + H]⁺ = 324.2, [M + 2H]²⁺ = 162.7 ¹H NMR (400 MHz, DMSO-d₆) δ ppm 0.69- 0.77 (m, 2 H), 0.96-1.04 (m, 2 H), 1.95- 2.18 (m, 5 H), 2.94 (td, J = 11.7, 1.5 Hz, 2 H), 3.31 (br d, J = 12.7 Hz, 2 H), 4.41 (tt, J = 11.0, 4.2 Hz, 1 H), 7.21 (d, J = 1.2 Hz, 1 H), 7.66 (d, J = 1.0 Hz, 1 H), 7.77 (s, 1 H), 8.06 (d, J = 1.2 Hz, 1 H), 8.18 (s, 1 H), 8.30 (br s, 2 H), 9.70 (s, 1 H). |
| Int. D-5h | TBM-8 | 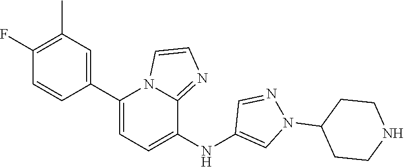 [SMILES: Cc1cc(-c2ccc(Nc3cnn(C4CCNCC4)c3)c3nccn23)ccc1F] | quant. yield, yellow solid [M + H]⁺ = 392.4 |
| Int. D-5i | TBM-9 | 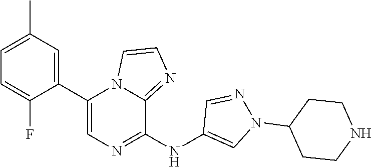 [SMILES: Cc1ccc(F)c(-c2cnc(Nc3cnn(C4CCNCC4)c3)c3nccn23)c1] | 84% yield, light grey solid [M + H]⁺ = 392.2 ¹H NMR (400 MHz, DMSO-d₆) δ ppm 2.19 (br s, 4 H), 2.37 (br s, 3 H), 3.04 (br d, J = 1.2 Hz, 2 H), 3.34-3.46 (m, 2 H), 4.47- 4.64 (m, 1 H), 7.34-7.47 (m, 3 H), 7.54- 7.61 (m, 1 H), 7.74-7.84 (m, 2 H), 7.90 (br s, 1 H), 8.23 (br s, 1 H), 8.95-9.08 (m, 1 H), 9.21-9.38 (m, 1 H), 10.66-10.81 (m, 1 H). |
| Int. D-5j | TBM-10 | 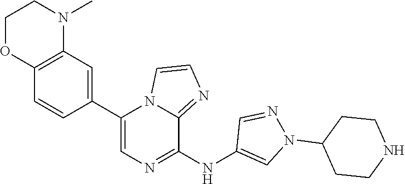 [SMILES: CN1CCOc2ccc(-c3cnc(Nc4cnn(C5CCNCC5)c4)c4nccn34)cc21] | 90% yield, tan solid [M + H]⁺ = 431.4 ¹H NMR (400 MHz, DMSO-d₆) δ ppm 2.11- 2.26 (m, 4 H), 2.89 (s, 3 H), 3.06 (br d, J = 8.8 Hz, 2 H), 3.28-3.35 (m, 2 H), 3.40 (br d, J = 12.7 Hz, 2 H), 4.26-4.35 (m, 2 H), 4.53 (dt, J = 9.7, 4.8 Hz, 1 H), 6.86 (s, 2 H), 6.92 (s, 1 H), 7.49 (s, 1 H), 7.76-7.84 (m, 1 H), 7.97 (s, 1 H), 8.07 (d, J = 1.0 Hz, 1 H), 8.22 (s, 1 H), 8.81-8.99 (m, 1 H), 9.07-9.20 (m, 1 H), 10.70-10.87 (m, 1 H). |
| Int. D-5k | TBM-11 | 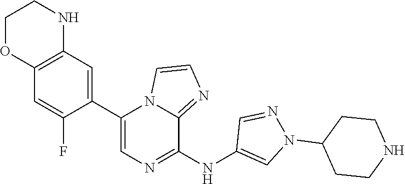 [SMILES: Fc1cc2c(cc1-c1cnc(Nc3cnn(C4CCNCC4)c3)c3nccn13)NCCO2] | quant. yield, light yellow solid [M + H]⁺ = 435.2 ¹H NMR (400 MHz, DMSO-d₆) δ ppm 2.10- 2.23 (m, 4 H), 2.97-3.15 (m, 2 H), 3.31- 3.44 (m, 4 H), 4.18-4.28 (m, 2 H), 4.48- 4.58 (m, 1 H), 6.78 (d, J = 7.3 Hz, 1 H), 6.83 (d, J = 10.8 Hz, 1 H), 7.39-7.48 (m, 1 H), 7.72-7.92 (m, 3 H), 8.23 (s, 1 H), 8.77- 8.92 (m, 1 H), 9.05-9.17 (m, 1 H), 10.56- 10.70 (m, 1 H). |
| Int. D-51 | TBM-12 | 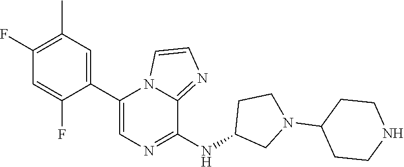 [SMILES: Cc1cc(-c2cnc(N[C@@H]3CCN(C4CCNCC4)C3)c3nccn23)c(F)cc1F] | 66% yield, white solid [M + H]⁺ = 413.2 ¹H NMR (400 MHz, DMSO-d₆) δ ppm 1.13- 1.30 (m, 2 H), 1.70-1.80 (m, 2 H), 1.83- 1.93 (m, 1 H), 2.03-2.11 (m, 1 H), 2.12- 2.23 (m, 1 H), 2.24-2.31 (m, 3 H), 2.41 (td, J = 11.9, 1.6 Hz, 2 H), 2.52-2.60 (m, 2 H), 2.65-2.76 (m, 1 H), 2.86-2.95 (m, 3 H), 4.52-4.63 (m, 1 H), 7.31 (s, 1 H), 7.37 (d, J = 7.3 Hz, 1 H), 7.42 (t, J = 9.9 Hz, 1 H), 7.52-7.58 (m, 3 H). ¹⁹F NMR (377 MHz, DMSO-d₆) δ ppm −112.16 (q, J = 8.2 Hz, 1 F), −111.48 (q, J = 8.2 Hz, 1 F). |
| Int. D-5m | TBM-13 | 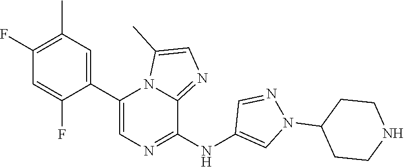 [SMILES: Cc1cc(-c2cnc(Nc3cnn(C4CCNCC4)c3)c3ncc(C)n23)c(F)cc1F] | 99% yield, brown solid [M + H]⁺ = 425.2 ¹H NMR (400 MHz, DMSO-d₆) δ ppm 1.91 (s, 3 H), 2.11-2.22 (m, 4 H), 2.29 (s, 3 H), 3.00-3.12 (m, 2 H), 3.39 (br s, 1 H), 3.43 (br s, 1 H), 4.49-4.55 (m, 1 H), 7.28 (s, 1 H), 7.39-7.50 (m, 2 H), 7.57 (t, J = 8.3 Hz, 1 H), 7.83 (s, 1 H), 8.23 (s, 1 H), 8.58- 8.74 (m, 1 H), 8.86-9.00 (m, 1 H), 10.23 (s, 1 H). ¹⁹F NMR (377 MHz, DMSO-d₆) δ ppm −113.02-−112.90 (m, 1 F), −110.55- −110.38 (m, 1 F) |
| Int. D-5n | TBM-14 | 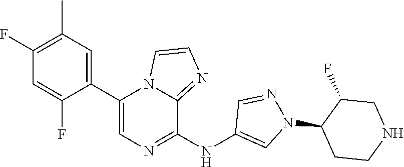 [SMILES: Cc1cc(-c2cnc(Nc3cnn([C@@H]4CCNC[C@H]4F)c3)c3nccn23)c(F)cc1F] | 89% yield, light-yellow solid [M + H]⁺ = 428.2 ¹H NMR (400 MHz, DMSO-d₆) δ ppm 2.24- 2.31 (m, 6 H), 3.04-3.26 (m, 2 H), 3.38- 3.46 (m, 1 H), 3.67-3.77 (m, 1 H), 5.05- 5.24 (m, 1 H), 7.42-7.51 (m, 2 H), 7.70 (br d, J = 10.3 Hz, 2 H), 7.91 (s, 1 H), 8.32 (s, 1 H), 9.34 (br s, 1 H), 9.60 (s, 1 H), 10.28 (s, 1 H). |
| Int. D-50 | TBM-15 | 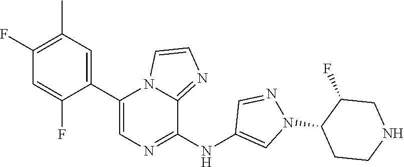 [SMILES: Cc1cc(-c2cnc(Nc3cnn([C@H]4CCNC[C@H]4F)c3)c3nccn23)c(F)cc1F] | quant. yield, yellow solid [M + H]⁺ = 428.2 ¹H NMR (400 MHz, DMSO-d₆) δ ppm 0.98 -1.46 (m, 1 H), 2.29 (s, 3 H), 3.31-3.54 (m, 3 H), 3.66 (br s, 2 H), 4.71-4.90 (m, 1 H), 5.16-5.40 (m, 1 H), 7.40-7.55 (m, 2 H), 7.60 (br s, 1 H), 7.68-7.81 (m, 2 H), 7.87 (s, 1 H), 8.28 (s, 1 H), 8.78-9.03 (m, 1 H). One H not observed. ¹⁹F NMR (377 MHz, DMSO-d₆) δ ppm −200.92-−199.75 (m, 1 F), −112.58- −111.53 (m, 1 F), −111.11-−110.48 (m, 1 F). |
| Int. D-5p | TBM-16 | 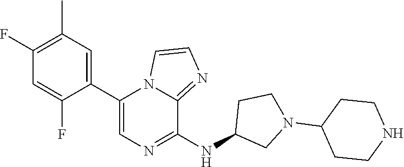 [SMILES: Cc1cc(-c2cnc(N[C@H]3CCN(C4CCNCC4)C3)c3nccn23)c(F)cc1F] | 90% yield, white solid, [M + H]⁺ = 413.2 ¹H NMR (400 MHz, DMSO-d₆) δ ppm 1.43- 1.57 (m, 2 H), 1.83-1.96 (m, 3 H), 2.14- 2.31 (m, 5 H), 2.52-2.63 (m, 2 H), 2.69- 2.79 (m, 3 H), 2.93 (dd, J = 8.8, 7.6 Hz, 1 H), 3.13 (br d, J = 12.0 Hz, 2 H), 4.55- 4.66 (m, 1 H), 7.31 (s, 1 H), 7.39-7.49 (m, 2 H), 7.52-7.59 (m, 3 H), 8.36 (s, 1 H). ¹⁹F NMR (377 MHz, DMSO-d₆) δ ppm −112.19 (d, J = 8.2 Hz, 1 F), −111.44 (d, J = 8.2 Hz, 1 F). |
| Int. D-5q | TBM-17 | 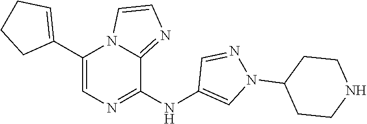 [SMILES: C1=C(c2cnc(Nc3cnn(C4CCNCC4)c3)c3nccn23)CCC1] | 75% yield, tan solid [M + H]⁺ = 350.2 |
| Int. D-5r | TBM-18 | 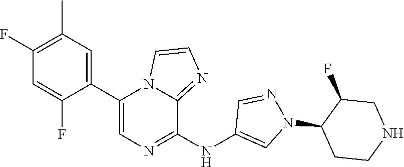 [SMILES: Cc1cc(-c2cnc(Nc3cnn([C@@H]4CCNC[C@@H]4F)c3)c3nccn23)c(F)cc1F] | 50% yield, brown solid [M + H]⁺ = 428.2 ¹H NMR (400 MHz, DMSO-d₆) δ ppm 1.95 (br d, J = 9.7 Hz, 1 H), 2.12-2.26 (m, 1 H), 2.29 (s, 3 H), 2.66-2.82 (m, 1 H), 2.86- 3.07 (m, 1 H), 3.10-3.33 (m, 2 H), 4.44- 4.67 (m, 1 H), 4.82-5.06 (m, 1 H), 7.39- 7.52 (m, 2 H), 7.57-7.68 (m, 3 H), 7.85 (s, 1 H), 8.15-8.24 (m, 1 H), 8.27 (s, 1 H), 10.04 (s, 1 H). ¹⁹F NMR (377 MHz, DMSO-d₆) δ ppm −201.96-−199.48 (m, 1 F), −112.13 (q, J = 8.6 Hz, 1 F), −111.24 (g, J = 9.1 Hz, 1 F). |
| Int. D-5s | TBM-19 | 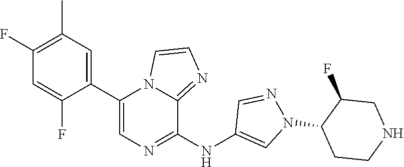 [SMILES: Cc1cc(-c2cnc(Nc3cnn([C@H]4CCNC[C@@H]4F)c3)c3nccn23)c(F)cc1F] | quant. yield, brown oil [M + H]⁺ = 428.2 ¹H NMR (400 MHz, DMSO-d₆) δ ppm 1.00- 1.44 (m, 1 H), 2.27 (br s, 3 H), 3.25-3.51 (m, 3 H), 3.52-3.74 (m, 2 H), 4.70-4.91 (m, 1 H), 5.10-5.36 (m, 1 H), 7.13-7.39 (m, 2 H), 7.42-7.72 (m, 2 H), 7.77-8.13 (m, 3 H), 8.22-8.34 (m, 1 H). 1 H not observed. ¹⁹F NMR (377 MHz, DMSO-d₆) δ ppm −188.68 (br d, J = 47.7 Hz, 1 F), −112.01- −111.62 (m, 1 F), −110.27-−109.81 (m, 1 F). |
| Int. D-5t | TBM-20 | 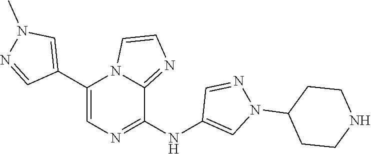 [SMILES: Cn1cc(-c2cnc(Nc3cnn(C4CCNCC4)c3)c3nccn23)cn1] | 67% yield, tan semi-solid [M + H]⁺ = 364.2 ¹H NMR (400 MHz, DMSO-d₆) δ ppm 1.76 (qd, J = 11.9, 3.9 Hz, 2 H), 1.94 (br d, J = 10.3 Hz, 2 H), 2.59 (br t, J = 11.5 Hz, 2 H), 2.97-3.08 (m, 2 H), 3.94 (s, 3 H), 4.06- 4.13 (m, 1 H), 4.17 (tt, J = 11.6, 4.1 Hz, 1 H), 7.53 (s, 1 H), 7.68 (d, J = 0.9 Hz, 1 H), 7.77 (s, 1 H), 7.94 (s, 1 H), 8.04 (d, J = 1.0 Hz, 1 H), 8.20 (s, 1 H), 8.30 (s, 1 H), 9.79 (s, 1 H). |
| Int. D-31 | TBM-21 | 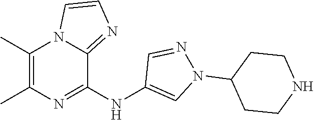 [SMILES: Cc1nc(Nc2cnn(C3CCNCC3)c2)c2nccn2c1C] | 82% yield, tan solid [M + H]⁺ = 312.2 ¹H NMR (400 MHz, DMSO-d₆) δ ppm 1.75 (qd, J = 12.0, 4.3 Hz, 2 H), 1.86-1.98 (m, 2 H), 2.00-2.19 (m, 1 H), 2.37 (s, 3 H), 2.44 (s, 3 H), 2.54-2.65 (m, 2 H), 3.03 (br d, J = 12.7 Hz, 2 H), 4.15 (tt, J = 11.6, 4.0 Hz, 1 H), 7.58 (s, 1 H), 7.77 (s, 1 H), 7.84 (d, J = 1.0 Hz, 1 H), 8.20 (s, 1 H), 9.52 (s, 1 H). |
| Int. D-5u | TBM-22 | 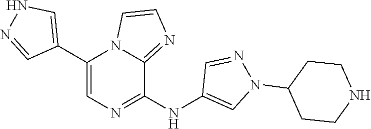 [SMILES: c1cn2c(-c3cn[nH]c3)cnc(Nc3cnn(C4CCNCC4)c3)c2n1] | quant. yield, yellow solid [M+H] = 350.2 ¹H NMR (400 MHz, DMSO-d₆) δ ppm 2.14- 2.26 (m, 4 H), 3.00-3.12 (m, 2 H), 3.35- 3.44 (m, 2 H), 4.48-4.59 (m, 1 H), 7.69 (br s, 1 H), 7.78 (s, 1 H), 8.10 (s, 1 H), 8.17- 8.25 (m, 3 H), 8.29 (s, 1 H), 8.94-9.11 (m, 1 H), 9.18-9.30 (m, 1 H), 10.87 (br s, 1 H). One proton not observed. |
| Int. D-5v | TBM-23 | 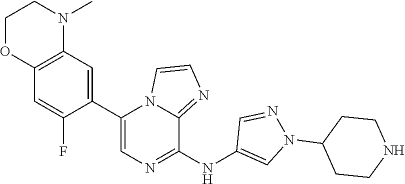 [SMILES: CN1CCOc2cc(F)c(-c3cnc(Nc4cnn(C5CCNCC5)c4)c4nccn34)cc21] | quant yield, yellow solid |
| Int. D-5w | TBM-24 | 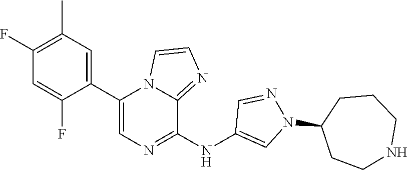 [SMILES: Cc1cc(-c2cnc(Nc3cnn([C@H]4CCCNCC4)c3)c3nccn23)c(F)cc1F] | quant yield, yellow solid [M + H]⁺ = 424.2 ¹H NMR (400 MHz, DMSO-d₆) δ ppm 1.76- 1.92 (m, 1 H), 1.92-2.04 (m, 1 H), 2.06- 2.21 (m, 2 H), 2.24-2.28 (m, 1 H), 2.30 (s, 3 H), 3.09-3.28 (m, 3 H), 3.30-3.38 (m, 1 H), 4.53-4.61 (m, 5 H), 7.44-7.51 (m, 2 H), 7.61 (t, J = 8.4 Hz, 1 H), 7.67-7.70 (m, 1 H), 7.71 (s, 1 H), 7.82 (s, 1 H), 8.25 (s, 1 H). ¹⁹F NMR (377 MHz, DMSO-d₆) δ ppm −112.16 (q, J = 8.6 Hz, 1 F), −111.02- −110.88 (m, 1 F). |
| Int. D-5x | TBM-25 | 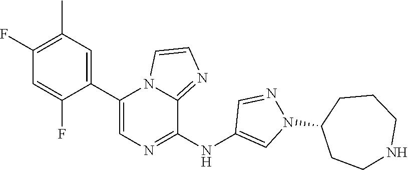 [SMILES: Cc1cc(-c2cnc(Nc3cnn([C@@H]4CCCNCC4)c3)c3nccn23)c(F)cc1F] | quant. yield, yellow solid [M + H]⁺ = 424.2 ¹H NMR (400 MHz, DMSO-d₆) δ ppm 1.78- 1.90 (m, 1 H), 1.93-2.04 (m, 1 H), 2.11- 2.22 (m, 2 H), 2.23-2.29 (m, 1 H), 2.30 (s, 3 H), 2.32-2.39 (m, 1 H), 3.11-3.23 (m, 3 H), 3.29-3.38 (m, 1 H), 4.53-4.64 (m, 1 H), 7.46-7.54 (m, 2 H), 7.61 (t, J = 8.2 Hz, 1 H), 7.76-7.85 (m, 3 H), 8.24 (s, 1 H), 9.01 (br s, 1 H), 9.16 (br s, 1 H), 10.53 (br s, 1 H). ¹⁹F NMR (377 MHz, DMSO-d₆) δ ppm −112.05 (g, J = 8.6 Hz, 1 F), −110.72- −110.51 (m, 1 F). |
| Int. D-5y | TBM-26 | 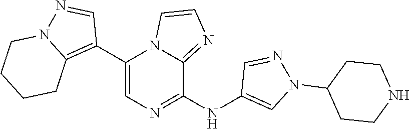 [SMILES: c1cn2c(-c3cnn4c3CCCC4)cnc(Nc3cnn(C4CCNCC4)c3)c2n1] | 96% yield, light-yellow solid [M + H]⁺ = 404.2 ¹H NMR (400 MHz, DMSO-d₆) δ ppm 1.76- 1.86 (m, 2 H), 1.99-2.08 (m, 2 H), 2.15- 2.25 (m, 4 H), 2.74 (t, J = 6.2 Hz, 2 H), 2.99-3.12 (m, 2 H), 3.34-3.44 (m, 2 H), 4.17 (t, J = 6.0 Hz, 2 H), 4.48-4.59 (m, 1 H), 7.44-7.48 (m, 1 H), 7.78 (s, 1 H), 7.81 (s, 1 H), 8.02 (s, 2 H), 8.21 (s, 1 H), 8.88- 9.04 (m, 1 H), 9.20 (br d, J = 10.3 Hz, 1 H), 10.85 (br s, 1 H). |
| Int. D-5z | TBM-27 | 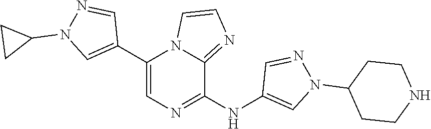 [SMILES: c1cn2c(-c3cnn(C4CC4)c3)cnc(Nc3cnn(C4CCNCC4)c3)c2n1] | quant. yield, light-yellow semi-solid [M + H]⁺ = 391.2 ¹H NMR (400 MHz, DMSO-d₆) δ ppm 1.00- 1.06 (m, 2 H), 1.14-1.19 (m, 2 H), 2.14- 2.23 (m, 4 H), 3.00-3.12 (m, 2 H), 3.36- 3.44 (m, 2 H), 3.84 (tt, J = 7.4, 3.9 Hz, 1 H), 4.49-4.56 (m, 1 H), 7.63 (br s, 1 H), 7.79 (s, 1 H), 7.96 (d, J = 0.7 Hz, 1 H), 8.00 (br s, 1 H), 8.20 (s, 1 H), 8.25-8.28 (m, 1 H), 8.42 (s, 1 H), 8.83-8.95 (m, 1 H), 9.05- 9.17 (m, 1 H), 10.63 (br s, 1 H). |
| Int. D-5aa | TBM-39 | 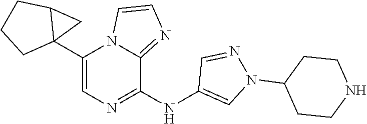 [SMILES: c1cn2c(C34CCCC3C4)cnc(Nc3cnn(C4CCNCC4)c3)c2n1] | 85% yield, white semi-solid [M + H]⁺ = 364.2 |

### Example S14. General Procedure for TBM-E

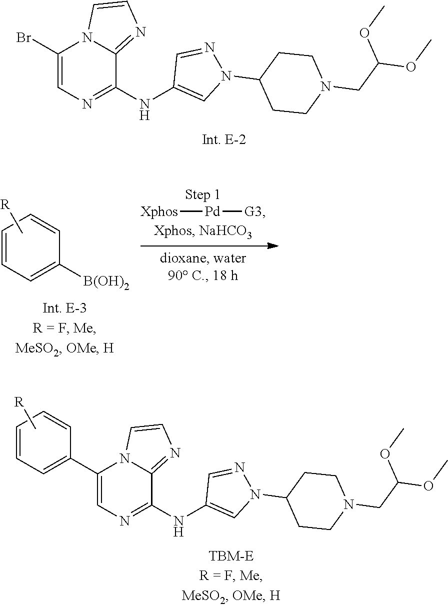

### Synthesis of TBM-E: Step 1

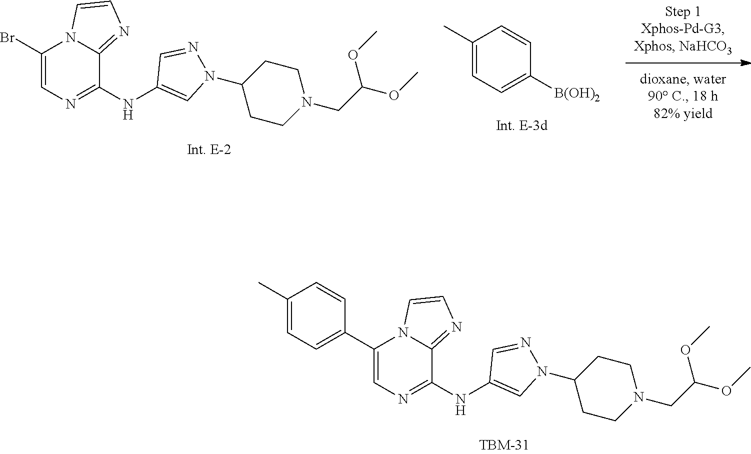

A solution of 5-bromo-N-(1-(1-(2,2-dimethoxyethyl)piperidin-4-yl)-1H-pyrazol-4-yl)imidazo[1,2-a]pyrazin-8-amine Int. E-2 (150 mg, 0.33 mmol), p-tolylboronic acid Int. E-3d (75 mg, 0.55 mmol), Xphos (61 mg, 0.05 mmol) and NaHCO₃ (85 mg, 1.01 mmol) in 1,4-dioxane (3 mL) and water (1 mL) was degassed by bubbling nitrogen for 10 minutes. Xphos-Pd-G3 (44 mg, 0.05 mmol) was added, and the mixture was bubbled with nitrogen for a further 10 minutes. The reaction mixture was then heated to 90° C. for 18 h. The reaction mixture was then cooled down to room temperature, filtered on a pad of Celite, rinsed with EtOAc and concentrated. The residue was purified by reverse phase column chromatography (50 to 100% MeCN in water w/ 0.1% formic acid) The fractions were collected and afforded N-(1-(1-(2,2-dimethoxyethyl)piperidin-4-yl)-1H-pyrazol-4-yl)-5-(p-tolyl)imidazo[1,2-a]pyrazin-8-amine TBM-31 (126 mg, 0.27 mmol, 82 yield) as a white semi-solid.

LCMS: [M+H]⁺=462.3.

¹H NMR (400 MHz, DMSO-d₆) δ ppm 1.88-2.01 (m, 4H), 2.16-2.25 (m, 2H), 2.40 (s, 3H), 2.47 (br d, J=5.3 Hz, 2H), 2.99 (br d, J=11.6 Hz, 2H), 3.27 (s, 6H), 4.11 (tat, J=10.5, 5.2 Hz, 1H), 4.49 (t, J=5.1 Hz, 1H), 7.38 (d, J=7.8 Hz, 2H), 7.41 (s, 1H), 7.57 (d, J=7.9 Hz, 2H), 7.63 (d, J=1.0 Hz, 1H), 7.79 (s, 1H), 7.86 (d, J=1.0 Hz, 1H), 8.24 (s, 1H), 9.86 (s, 1H).

TABLE 21
List of intermediates used in TBM-E: Step 1
Starting Material

| Int. E | Structure | Source |
|---|---|---|
| Int. E-2 | 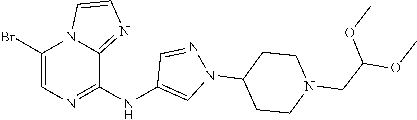 [SMILES: COC(CN1CCC(n2cc(Nc3ncc(Br)n4ccnc34)cn2)CC1)OC] | Synthesis provided below |
| Int. E-3a | 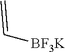 | commercially available |
| Int. E-3b | 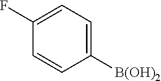 | commercially available |
| Int. E-3c | 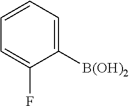 | commercially available |
| Int. E-3d | 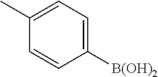 | commercially available |
| Int. E-3e | 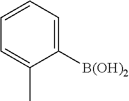 | commercially available |
| Int. E-3f | 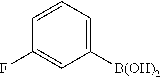 | commercially available |
| Int. E-3g | 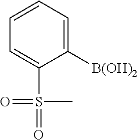 | commercially available |
| Int. E-3h | 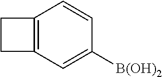 [SMILES: OB(O)c1ccc2c(c1)CC2] | commercially available |
| Int. E-3i | 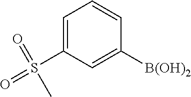 | commercially available |
| Int. E-3j | 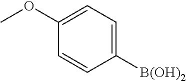 | commercially available |
| Int. E-3k | 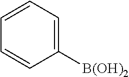 | commercially available |
| Int. E-31 | 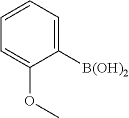 | commercially available |
| Int. E-3m | 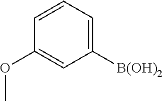 | commercially available |
| Int. E-3n | 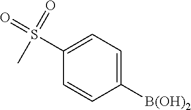 | commercially available |

### Synthesis of 5-bromo-N-(1-(1-(2,2-dimethoxyethyl)piperidin-4-yl)-1H-pyrazol-4-yl)imidazo[1,2-a]pyrazin-8-amine (Int. E-2)

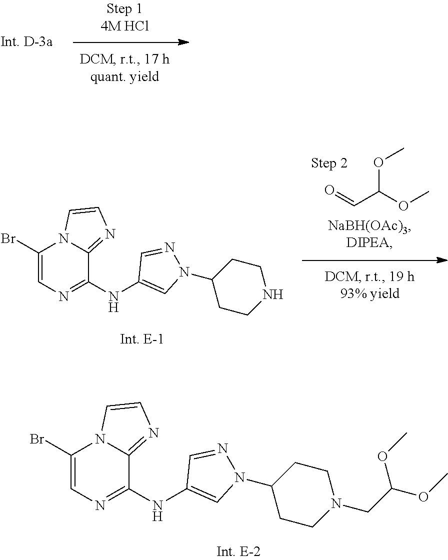

Step 1: To a solution of tert-butyl 4-(4-((5-bromoimidazo[1,2-a]pyrazin-8-yl)amino)-1H-pyrazol-1-yl)piperidine-1-carboxylate Int. D-3a (7.15 g, 15.45 mmol) in DCM (150 mL) was added 4 M HCl in 1,4 dioxane (38.0 mL, 152 mmol). After 17 h at room temperature, the reaction mixture was concentrated in vacuo to afford 5-bromo-N-(1-(piperidin-4-yl)-1H-pyrazol-4-yl)imidazo[1,2-a]pyrazin-8-amine Int. E-1 (7.26 g, 19.86 mmol, quant.) as a brown oil. The product was used in the next step without further purification.

LCMS: [M+H]⁺=362.0.

¹H NMR (400 MHz, DMSO-d₆) δ ppm 2.11-2.22 (m, 4H), 2.97-3.10 (m, 2H), 3.37 (br d, J=12.6 Hz, 2H), 4.51 (br t, J=7.3 Hz, 1H), 7.68 (s, 1H), 7.79 (s, 1H), 7.87 (d, J=0.9 Hz, 1H), 8.09 (d, J=1.2 Hz, 1H), 8.17 (s, 1H), 10.38 (s, 1H). One proton was not observed.

Step 2: To solution of 5-bromo-N-(1-(piperidin-4-yl)-1H-pyrazol-4-yl)imidazo[1,2-a]pyrazin-8-amine Int. E-1 (5.6 g, 15.45 mmol), DIPEA (10.0 mL, 57.41 mmol) and 2,2-dimethoxyacetaldehyde 60% wt. in H₂O (0.77 mL, 53 mmol) in DCM (150 mL) was added NaBH(OAc)₃ (6.64 g, 31.3 mmol). After 19 h at rt, the reaction mixture was concentrated and purified by reverse phase column chromatography (500 to 100% MeOH in water w/0.100 formic acid). The fractions were collected and afforded 5-bromo-N-(1-(1-(2,2-dimethoxyethyl)piperidin-4-yl)-1H-pyrazol-4-yl)imidazo[1,2-a]pyrazin-8-amine Int. E-2 (6.60 g, 14.40 mmol, 930% yield) as a brown oil.

LCMS: [M+H]⁺=516.2.

¹H NMR (400 MHz, DMSO-d₆) δ ppm 1.81-1.99 (NM, 4H), 2.18-2.31 (m, 2H), 2.50-2.53 (m, 1H), 3.01 (br d, J=11.6 Hz, 2H), 3.27 (s, 6H), 4.11 (tt, J=10.4, 5.1 Hz, 1H), 4.50 (t, J 5.1 Hz, 1H), 7.59 (s, 1H), 7.70 (d, J=1.0 Hz, 1H), 7.75 (s, 1H), 7.98 (d, J=1.0 Hz, 1H), 8.13-8.17 (m, 1H), 9.96-10.06 (m, 1H). One proton was not observed.

TABLE 22
List of TBM-E compounds generated by Representative Procedure TBM-E: Step 1

| Starting | Product | | | |
|---|---|---|---|---|
| Material | TBM | | | |
| Int. E-3 | # | Structure | Characterization | Notes |
| Int. E-3a | TBM- 28′ | 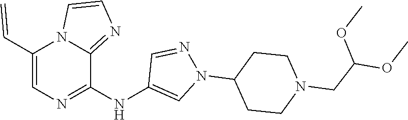 [SMILES: C=Cc1cnc(Nc2cnn(C3CCN(CC(OC)OC)CC3)c2)c2nccn12] | 30% yield, tan solid [M + H]⁺ = 398.3 ¹H NMR (400 MHz, DMSO-d₆) δ ppm 1.86-2.00 (m, 5 H), 2.15-2.24 (m, 3 H), 2.98 (br d, J = 11.7 Hz, 2 H), 3.27-3.28 (m, 6 H), 4.05-4.17 (m, 1 H), 4.49 (t, J = 5.1 Hz, 1 H), 5.40 (d, J = 11.7 Hz, 1 H), 5.95 (d, J = 17.6 Hz, 1 H), 6.97 (dd, J = 17.6, 11.5 Hz, 1 H), 7.66-7.69 (m, 1 H), 7.70-7.72 (m, 1 H), 7.77-7.79 (m, 1 H), 8.20-8.22 (m, 1 H), 8.23-8.25 (m, 1 H), 9.92 (s, 1 H). | Conditions: Xphos-Pd-G2, Xphos, K₃PO₄, THE, 60 °C, 20 h TBM-28' was converted to TBM-28 by a one-step protocol provided below |
| Int. E-3b | TBM- 29 | 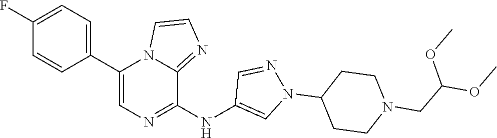 [SMILES: COC(CN1CCC(n2cc(Nc3ncc(-c4ccc(F)cc4)n4ccnc34)cn2)CC1)OC] | 60% yield, yellow oil [M + H]⁺ = 466.2 ¹H NMR (400 MHz, DMSO-d₆) δ ppm 1.91-2.04 (m, 4 H), 2.23-2.41 (m, 2 H), 2.55-2.62 (m, 2 H), 3.03-3.11 (m, 2 H), 3.29 (s, 6 H), 4.08- 4.23 (m, 1 H), 4.53 (t, J = 4.9 Hz, 1 H), 7.08-7.19 (m, 2 H), 7.42 (s, 1 H), 7.65 (d, J = 1.0 Hz, 1 H), 7.74 (dd, J = 8.7, 5.5 Hz, 2 H), 7.85 (br d, J = 0.9 Hz, 1 H), 8.13 (s, 1 H), 8.24 (s, 1 H), 9.92 (s, 1 H). One proton not observed. | Conditions: Pd(PPh₃)₄ NaHCO₃ dioxane/water 90° C., 6 h |
| Int. E-3c | TBM- 30 | 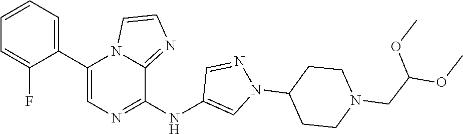 [SMILES: COC(CN1CCC(n2cc(Nc3ncc(-c4ccccc4F)n4ccnc34)cn2)CC1)OC] | 51% yield, yellow oil [M + H]⁺ = 466.1 ¹H NMR (400 MHz, DMSO-d₆) δ ppm 1.86-2.00 (m, 4 H), 2.21 (br t, J = 10.5 Hz, 2 H), 3.00 (br d, J = 11.1 Hz, 2 H), 3.20 (br s, 2 H), 3.27 (s, 6 H), 4.07-4.21 (m, 1 H), 4.49 (br t, J = 4.6 Hz, 1 H), 7.39-7.50 (m, 3 H), 7.58-7.68 (m, 3 H), 7.80 (s, 1 H), 8.09- 8.18 (m, 1 H), 8.25 (s, 1 H), 9.99 (s, 1 H). ¹⁹F NMR (377 MHz, DMSO-d₆) δ ppm −112.16 (br s, 1 F). | Conditions: Pd(PPh₃)₄ NaHCO₃ dioxane/water 90° C., 6 h |
| Int. E-3d | TBM- 31 | 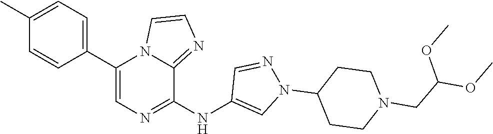 | 82% yield, white semi-solid [M + H]⁺ = 462.3 ¹H NMR (400 MHz, DMSO-d6) δ ppm 1.88-2.01 (m, 4 H), 2.16-2.25 (m, 2 H), 2.40 (s, 3 H), 2.47 (br d, J = 5.3 Hz, 2 H), 2.99 (br d, J = 11.6 Hz, 2 H), 3.27 (s, 6 H), 4.11 (tt, J = 10.5, 5.2 Hz, 1 H), 4.49 (t, J = 5.1 Hz, 1 H), 7.38 (d, J = 7.8 Hz, 2 H), 7.41 (s, 1 H), 7.57 (d, J = 7.9 Hz, 2 H), 7.63 (d, J = 1.0 Hz, 1 H), 7.79 (s, 1 H), 7.86 (d, J = 1.0 Hz, 1 H), 8.24 (s, 1 H), 9.86 (s, 1 H). | Conditions: Xphos-Pd-G3, Xphos, NaHCO₃, dioxane/water, 90 °C, 18 h |
| Int. E-3e | TBM- 32 | 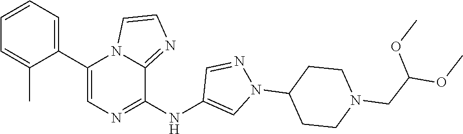 [SMILES: COC(CN1CCC(n2cc(Nc3ncc(-c4ccccc4C)n4ccnc34)cn2)CC1)OC] | 71% yield, colorless semi-solid [M + H]⁺ = 462.3 ¹H NMR (400 MHz, DMSO-d₆) δ ppm 1.86-2.02 (m, 3 H), 2.04-2.16 (m, 3 H), 2.17-2.27 (m, 2 H), 2.47 (br s, 2 H), 2.99 (br d, J = 11.0 Hz, 2 H), 3.27 (s, 7 H), 4.11 (td, J = 10.5, 5.5 Hz, 1 H), 4.46- 4.53 (m, 1 H), 7.07- 7.23 (m, 1 H), 7.24- 7.49 (m, 5 H), 7.59 (s, 1 H), 7.74-7.85 (m, 1 H), 8.14 (s, 1 H), 8.25 (s, 1 H), 9.86 (s, 1 H). | Conditions: Xphos-Pd-G3, Xphos, NaHCO₃, dioxane/water, 90 °C, 2 h |
| Int. E-3f | TBM- 33 | 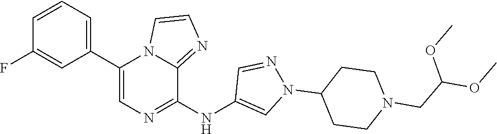 [SMILES: COC(CN1CCC(n2cc(Nc3ncc(-c4cccc(F)c4)n4ccnc34)cn2)CC1)OC] | 89% yield, yellow oil [M + H]⁺ = 466.2 ¹H NMR (400 MHz, DMSO-d₆) δ ppm 1.88-2.03 (m, 4 H), 2.27 (td, J = 11.4, 2.6 Hz, 2 H), 2.52 (d, J = 5.1 Hz, 2 H), 2.99-3.06 (m, 2 H), 3.27 (s, 6 H), 4.09- 4.17 (m, 1 H), 4.51 (br t, J = 5.1 Hz, 1 H), 7.30-7.39 (m, 1 H), 7.50 (s, 1 H), 7.52-7.64 (m, 3 H), 7.65 (d, J = 0.6 Hz, 1 H), 7.80 (s, 1 H), 7.95 (s, 1 H), 8.13 (s, 2 H), 8.24 (s, 1 H), 9.97 (s, 1 H). 2 H not observed. ¹⁹F NMR (377 MHz, DMSO-d₆) δ ppm −111.81 (td, J = 9.5, 5.4 Hz, 1 F). | Conditions: Xphos-Pd-G3, Xphos, NaHCO₃, dioxane/water, 90 ° C., 18 h |
| Int. E-3g | TBM- 34 | 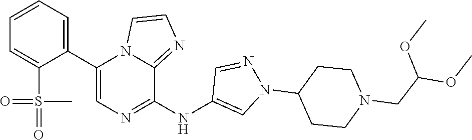 [SMILES: COC(CN1CCC(n2cc(Nc3ncc(-c4ccccc4S(C)(=O)=O)n4ccnc34)cn2)CC1)OC] | 53% yield, light yellow solid [M + H]⁺ = 526.2 ¹H NMR (400 MHz, DMSO-d₆) δ ppm 1.85-2.01 (m, 4 H), 2.14-2.24 (m, 2 H), 2.46 (d, J = 5.1 Hz, 2 H), 2.94-3.02 (m, 2 H), 3.09 (s, 3 H), 3.26 (s, 6 H), 4.07- 4.16 (m, 1 H), 4.48 (t, J = 5.1 Hz, 1 H), 7.15 (d, J = 1.0 Hz, 1 H), 7.43 (s, 1 H), 7.52 (d, J = 1.0 Hz, 1 H), 7.69 (dd, J = 7.2, 1.3 Hz, 1 H), 7.79 (s, 1 H), 7.85-7.95 (m, 2 H), 8.19 (dd, J = 7.6, 1.5 Hz, 1 H), 8.25 (s, 1 H), 9.92 (s, 1 H). | Conditions: Xphos-Pd-G3, Xphos, NaHCO₃, dioxane/water, 90 °C, 18 h |
| Int. E-3h | TBM- 35 | 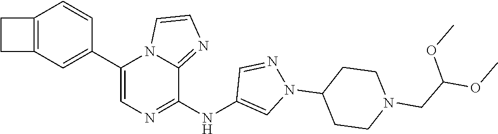 [SMILES: COC(CN1CCC(n2cc(Nc3ncc(-c4ccc5c(c4)CC5)n4ccnc34)cn2)CC1)OC] | 67% yield, light- yellow gum [M + H]⁺ = 474.2 ¹H NMR (400 MHz, DMSO-d₆) δ ppm 1.88-1.98 (m, 4 H), 2.17-2.27 (m, 2 H), 2.47-2.48 (m, 1 H), 2.95-3.04 (m, 2 H), 3.23 (s, 4 H), 3.26- 3.29 (m, 7 H), 4.07- 4.17 (m, 1 H), 4.50 (t, J = 5.0 Hz, 1 H), 7.27 (d, J = 7.6 Hz, 1 H), 7.34-7.39 (m, 2 H), 7.44-7.49 (m, 1 H), 7.62 (d, J = 1.0 Hz, 1 H), 7.78 (s, 1 H), 7.82 (d, J = 1.0 Hz, 1 H), 8.14 (s, 1 H), 8.24 (s, 1 H), 9.84 (s, 1 H). | Conditions: Xphos-Pd-G3, Xphos, NaHCO₃, dioxane/water, 90° C., 18 h |
| Int. E-3i | TBM- 36 | 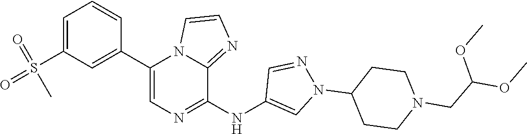 [SMILES: COC(CN1CCC(n2cc(Nc3ncc(-c4cccc(S(C)(=O)=O)c4)n4ccnc34)cn2)CC1)OC] | 67% yield, light yellow semi-solid [M + H]⁺ = 526.2 ¹H NMR (400 MHz, DMSO-d₆) δ ppm 1.87-2.02 (m, 4 H), 2.17-2.26 (m, 2 H), 2.47-2.49 (m, 2 H), 2.99 (br d, J = 11.7 Hz, 2 H), 3.27 (s, 6 H), 3.33 (s, 3 H), 4.07-4.17 (m, 1 H), 4.49 (t, J = 5.1 Hz, 1 H), 7.58 (s, 1 H), 7.69 (d, J = 1.2 Hz, 1 H), 7.80-7.87 (m, 2 H), 7.97 (d, J = 1.0 Hz, 1 H), 8.01-8.09 (m, 2 H), 8.14 (s, 1 H), 8.20 (br t, J = 1.7 Hz, 1 H), 8.25 (s, 1 H), 10.03 (s, 1 H). | Conditions: Xphos-Pd-G3, Xphos, NaHCO₃, dioxane/water, 90° C., 18 h |
| Int. E-3j | TBM- 37 | 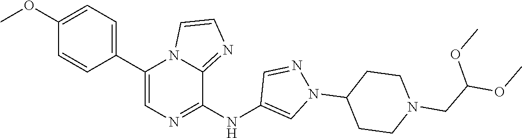 | 80% yield, colorless semi-solid [M + H]⁺ = 478.3 ¹H NMR (400 MHz, DMSO-d6) δ ppm 1.90-1.97 (m, 4 H), 2.18-2.23 (m, 2 H), 2.47-2.48 (m, 2 H), 2.99 (br d, J = 12.0 Hz, 2 H), 3.27 (s, 6 H), 3.84 (s, 3 H), 4.08-4.13 (m, 1 H), 4.49 (t, J = 5.0 Hz, 1 H), 7.12 (d, J = 8.6 Hz, 2 H), 7.37 (s, 1 H), 7.61 (d, J = 8.6 Hz, 2 H), 7.63 (d, J = 1.0 Hz, 1 H), 7.78 (s, 1 H), 7.83 (d, J = 1.0 Hz, 1 H), 8.23 (s, 1 H), 9.83 (s, 1 H). | Conditions: Xphos-Pd-G3, Xphos, NaHCO₃, dioxane/water, 90° C., 18 h |
| Int. E-3k | TBM- 38 | 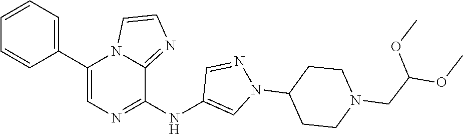 [SMILES: COC(CN1CCC(n2cc(Nc3ncc(-c4ccccc4)n4ccnc34)cn2)CC1)OC] | 92% yield, yellow oil [M + H]⁺ = 448.2 ¹H NMR (400 MHz, DMSO-d₆) δ ppm 1.86-2.02 (m, 4 H), 2.25 (td, J = 11.3, 2.3 Hz, 2 H), 3.01 (br d, J = 11.7 Hz, 2 H), 3.27 (s, 6 H), 4.06-4.18 (m, 1 H), 4.50 (t, J = 5.1 Hz, 1 H), 7.45 (s, 1 H), 7.48-7.54 (m, 1 H), 7.55-7.61 (m, 2 H), 7.64 (d, J = 0.9 Hz, 1 H), 7.69 (d, J = 7.2 Hz, 2 H), 7.80 (s, 1 H), 7.88 (d, J = 1.0 Hz, 1 H), 8.13 (s, 2 H), 8.25 (s, 1 H), 9.91 (s, 1 H). 4 protons not observed. | Conditions: Xphos-Pd-G3, Xphos, NaHCO₃, dioxane/water, 90° C., 18 h |
| Int. E-31 | TBM- 40 | 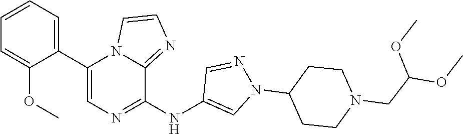 [SMILES: COc1ccccc1-c1cnc(Nc2cnn(C3CCN(CC(OC)OC)CC3)c2)c2nccn12] | 65% yield, yellow oil [M + H]⁺ = 479.2. ¹H NMR (400 MHz, DMSO-d₆) δ ppm 1.92-2.01 (m, 4 H), 2.28 (td, J = 11.3, 2.9 Hz, 2 H), 2.54- 2.55 (m, 2 H), 3.04 (br d, J = 11.7 Hz, 2 H), 3.28 (s, 6 H), 3.76 (s, 3 H), 4.10- 4.19 (m, 1 H), 4.52 (t, J = 5.1 Hz, 1 H), 7.11 (t, J = 7.3 Hz, 1 H), 7.23 (d, J = 8.3 Hz, 1 H), 7.32-7.37 (m, 2 H), 7.40-7.46 (m, 1 H), 7.51-7.59 (m, 2 H), 7.79 (s, 1 H), 8.13 (s, 2 H), 8.24 (s, 1 H), 9.83 (s, 1 H). 2 protons not observed. | Conditions: Xphos-Pd-G3, Xphos, NaHCO₃, dioxane/water, 90 °C, 18 h |
| Int. E-3m | TBM- 41 | 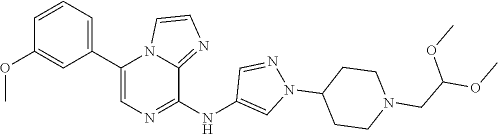 [SMILES: COc1cccc(-c2cnc(Nc3cnn(C4CCN(CC(OC)OC)CC4)c3)c3nccn23)c1] | 82% yield, yellow oil [M + H]⁺ = 479.2 ¹H NMR (400 MHz, DMSO-d₆) δ ppm 1.88-2.02 (m, 4 H), 2.25 (td, J = 11.5, 2.4 Hz, 2 H), 2.50- 2.54 (m, 2 H), 3.01 (br d, J = 11.7 Hz, 2 H), 3.27 (s, 6 H), 3.83 (s, 3 H), 4.07- 4.18 (m, 1 H), 4.50 (t, J = 5.1 Hz, 1 H), 7.07 (dd, J = 8.3, 1.8 Hz, 1 H), 7.20-7.27 (m, 2 H), 7.43-7.50 (m, 2 H), 7.64 (d, J = 0.9 Hz, 1 H), 7.79 (s, 1 H), 7.91-7.94 (m, 1 H), 8.13 (s, 2 H), 8.24 (s, 1 H), 9.90 (s, 1 H). 2 protons not observed. | Conditions: Xphos-Pd-G3, Xphos, NaHCO₃, dioxane/water, 90 °C, 18 h |
| Int. E-3n | TBM- 42 | 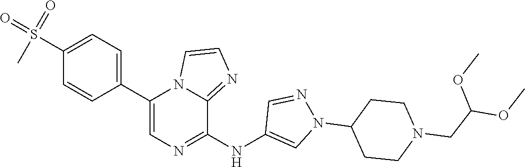 [SMILES: COC(CN1CCC(n2cc(Nc3ncc(-c4ccc(S(C)(=O)=O)cc4)n4ccnc34)cn2)CC1)OC] | 51% yield, yellow solid [M + H]⁺ = 526.2 ¹H NMR (400 MHz, DMSO-d₆) δ ppm 1.85-2.01 (m, 4 H), 2.15-2.25 (m, 2 H), 2.47 (d, J = 5.1 Hz, 2 H), 2.94-3.02 (m, 2 H), 3.27 (s, 6 H), 3.31 (s, 3 H), 4.07- 4.16 (m, 1 H), 4.49 (t, J = 5.1 Hz, 1 H), 7.58 (s, 1 H), 7.68 (d, J = 1.2 Hz, 1 H), 7.81 (s, 1 H), 7.96- 8.02 (m, 3 H), 8.06- 8.10 (m, 2 H), 8.25 (s, 1 H), 10.07 (s, 1 H). | Conditions: Xphos-Pd-G3, Xphos, NaHCO₃, dioxane/water, 90° C., 18 h |

### Synthesis of N-(1-(1-(2,2-dimethoxyethyl)piperidin-4-yl)-1H-pyrazol-4-yl)-5-ethylimidazo[1,2-a]pyrazin-8-amine (TBM-28) from TBM-28′

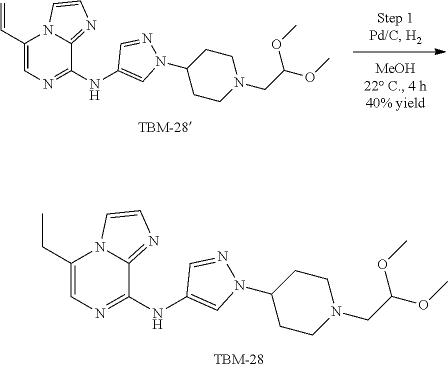 [SMILES: C=Cc1cnc(Nc2cnn(C3CCN(CC(OC)OC)CC3)c2)c2nccn12 ; CCc1cnc(Nc2cnn(C3CCN(CC(OC)OC)CC3)c2)c2nccn12]

To a solution of N-(1-(1-(2,2-dimethoxyethyl)piperidin-4-yl)-1H-pyrazol-4-yl)-5-vinylimidazo[1,2-a]pyrazin-8-amine TBM-28′ (115 mg, 0.17 mmol, 1 eq.) in methanol (2.9 mL) under N₂ was added Pd/C (17 mg, 0.02 mmol, 0.1 eq.) at room temperature, then H₂ was bubbled into the mixture and it was stirred under an H₂ atmosphere. The mixture was filtered through celite, rinsed with MeOH and concentrated. The residue was then purified by reverse phase column chromatography (5% to 100% MeOH in water w/ 0.1% formic acid). The fractions were combined and concentrated to give afford N-(1-(1-(2,2-dimethoxyethyl)piperidin-4-yl)-1H-pyrazol-4-yl)-5-ethylimidazo[1,2-a]pyrazin-8-amine TBM-28 (30 mg, 40% yield) as a light-yellow oil.

¹H NMR (400 MHz, DMSO-d₆) δ ppm 1.29 (t, J=7.5 Hz, 3H), 1.79-2.09 (m, 6H), 2.13-2.25 (m, 2H), 2.83 (q, J=7.3 Hz, 2H), 2.98 (br d, J=11.2 Hz, 2H), 3.27-3.27 (m, 6H), 4.03-4.16 (m, 1H), 4.49 (t, J=5.1 Hz, 1H), 7.24 (s, 1H), 7.63-7.66 (m, 1H), 7.74 (s, 1H), 7.95-7.98 (m, 1H), 8.17-8.19 (m, 1H), 9.59 (s, 1H).

### Example S15. General Procedure for TBM-F

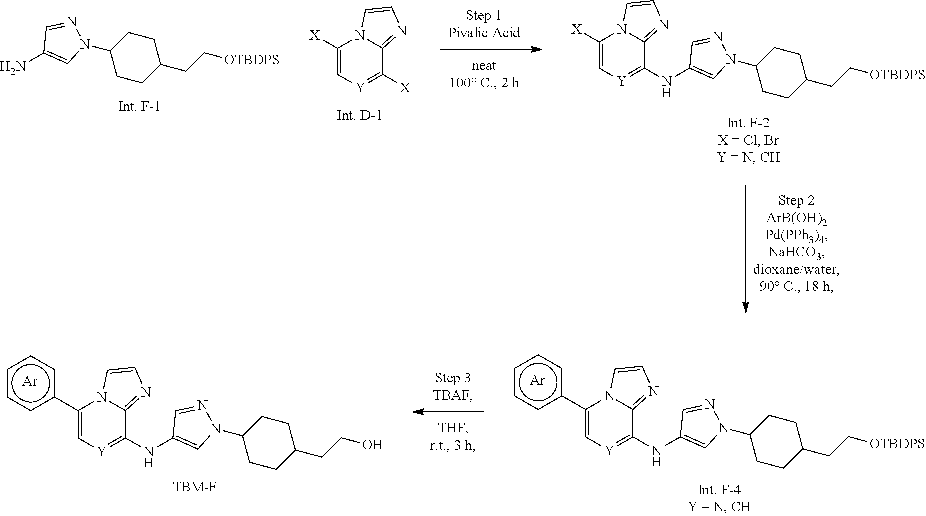

### Synthesis of TBM-F: Step 1

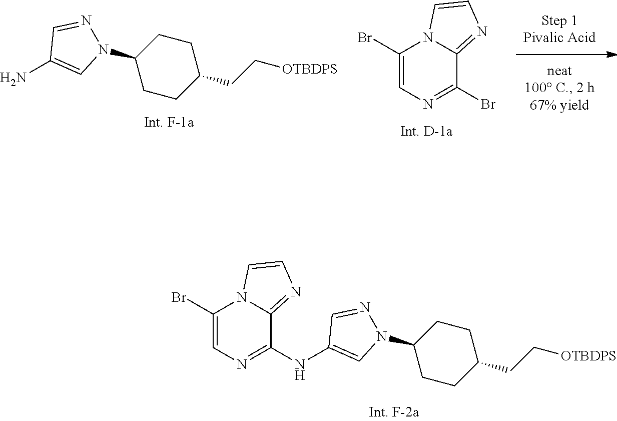

To a round bottom flask, was added 1-((trans)-4-(2-((tert-butyldiphenylsilyl)oxy)ethyl)cyclohexyl)-1H-pyrazol-4-amine Int. F-1a (1.64 g, 3.36 mmol, 1 eq.), 5,8-dibromoimidazo[1,2-a]pyrazine 6 (0.93 g, 3.36 mmol, 1 eq.) and pivalic acid (4.68 g, 45.8 mmol, 13.6 eq.). The reaction was placed in a pre-heated oil bath to 100° C. After 2 h, the reaction was quenched with a saturated solution of NaHCO₃ (until pH 7~9) and extracted with MeTHF (3×). The combined organic layers were washed with brine, dried over Na₂SO₄ and concentrated to give a dark red residue, which was then purified by reverse phase column chromatography (5% to 100% MeOH in water w/ 0.1% formic acid). The fractions were combined and concentrated to afford 5-bromo-N-(1-((trans)-4-(2-((tert-butyldiphenylsilyl)oxy)ethyl)cyclohexyl)-1H-pyrazol-4-yl)imidazo[1,2-a]pyrazin-8-amine Int. F-2a (1.56 g, 67% yield) as a light orange solid.

LCMS: [M+H]⁺=643.2, 645.2.

¹H NMR (400 MHz, DMSO-d₆) δ ppm 1.01 (s, 9H), 1.03-1.13 (m, 2H), 1.42-1.53 (m, 3H), 1.60-1.72 (m, 2H), 1.72-1.80 (m, 2H), 1.94-2.02 (m, 2H), 3.71 (br t, J=5.9 Hz, 2H), 4.00-4.08 (m, 1H), 7.42-7.50 (m, 6H), 7.59 (s, 1H), 7.60-7.65 (m, 4H), 7.70 (d, J=1.0 Hz, 1H), 7.73 (s, 1H), 7.98 (d, J=1.0 Hz, 1H), 8.13 (s, 1H), 9.97 (s, 1H).

TABLE 23
List of intermediates used in TBM-F: Step 1
Starting Material

| Int. | | |
|---|---|---|
| F-1 | Structure | Source |
| Int. F-1a | 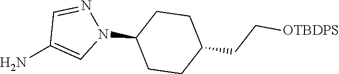 | Synthesis provided below |
| Int. F-1b | 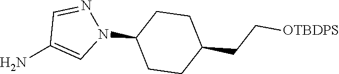 | Synthesis provided below |

### Synthesis of 1-((trans)-4-(2-((tert-butyldiphenylsilyl)oxy)ethyl)cyclohexyl)-1H-pyrazol-4-amine (Int. F-1a) and 1-((cis)-4-(2-((tert-butyldiphenylsilyl)oxy)ethyl)cyclohexyl)-1H-pyrazol-4-amine (Int. F-1b)

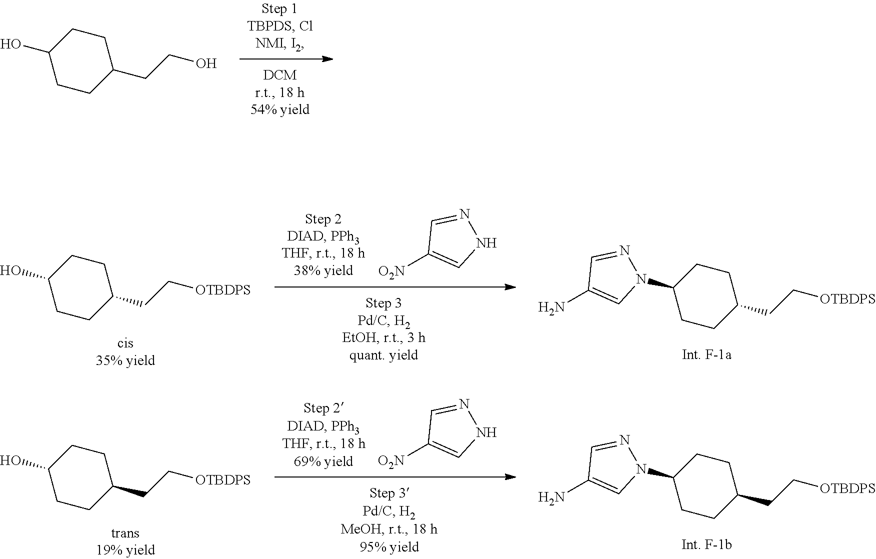

Step 1: To a solution of 4-(2-hydroxyethyl)cyclohexanol (5 g, 34.7 mmol, 1 eq.) in DCM (63 mL) was added N-methyl-imidazole (8.29 mL, 104 mmol, 3 eq.), iodine (17.6 g, 69 mmol, 2 eq.) and tert-butylchlorodiphenylsilane (11.72 mL, 45 mmol, 1.3 eq.). After stirring for 18 h at room temperature under nitrogen atmosphere, the reaction was washed with 10% sodium thiosulphate solution (2×80 mL) and brine (1×80 mL). The organic phase was then dried over sodium sulfate, filtered, and concentrated to give a thick orange oil. The residue was then purified by silica gel column chromatography (0% to 30% EtOAc in heptane). After two purifications by silica gel column chromatography, the fractions were combined and concentrated to afford (cis)-4-(2-((tert-butyldiphenylsilyl)oxy)ethyl)cyclohexan-1-ol cis (4.62 g, 35% yield) as a colorless oil and (trans)-4-(2-((tert-butyldiphenylsilyl)oxy)ethyl)cyclohexan-1-ol trans (2.6 g, 19% yield) as a colorless oil; with 54% combined yield.

cis: LCMS: [M+Na+H]⁺=405.4. ¹H NMR (400 MHz, DMSO-d₆) δ ppm 0.99 (s, 9 H), 1.25-1.40 (m, 6H), 1.42-1.54 (m, 5H), 3.64-3.71 (m, 3H), 4.20 (d, J=3.4 Hz, 1H), 7.40-7.48 (m, 6H), 7.58-7.63 (m, 4H).

trans: LCMS: [M+Na+H]⁺=405.4. ¹H NMR (400 MHz, DMSO-d₆) δ ppm 0.78-0.91 (m, 2H), 0.99 (s, 9H), 1.01-1.11 (m, 2H), 1.24-1.36 (m, 1H), 1.40 (q, J=6.4 Hz, 2H), 1.53-1.62 (m, 2H), 1.72-1.79 (m, 2H), 3.22-3.31 (m, 1H), 3.66 (t, J=6.5 Hz, 2H), 4.41 (d, J=4.6 Hz, 1H), 7.40-7.48 (m, 6H), 7.57-7.63 (m, 4H).

Step 2: To a solution of (cis)-4-(2-((tert-butyldiphenylsilyl)oxy)ethyl)cyclohexan-1-ol cis (2.2 g, 5.23 mmol, 1 eq.), triphenylphosphine (2058 mg, 7.85 mmol, 1.5 eq.) and 4-nitro-1H-pyrazole (887 mg, 7.9 mmol, 1.5 eq.) in anhydrous THE (10 mL) at 0° C. was slowly added diisopropyl azodicarboxylate (1.54 mL, 7.6 mmol, 1.5 eq.) as a solution in anhydrous THE (15 mL). The reaction was warmed to room temperature and stirred for 18 h. The reaction was concentrated and purified by reverse phase column chromatography (5% to 100% MeOH in water w/ 0.1% formic acid). The fractions were combined and concentrated to afford 1-((trans)-4-(2-((tert-butyldiphenylsilyl)oxy)ethyl)cyclohexyl)-4-nitro-1H-pyrazole (960 mg, 38% yield) as a colorless oil.

LCMS: [M+H]⁺=478.4.

¹H NMR (400 MHz, DMSO-d₆) δ ppm 1.00 (s, 9H), 1.02-1.12 (m, 2H), 1.43-1.54 (m, 3H), 1.67-1.81 (m, 4H), 2.01 (br d, J=11.0 Hz, 2H), 3.70 (t, J=5.7 Hz, 2H), 4.15-4.24 (m, 1H), 7.42-7.49 (m, 6H), 7.59-7.65 (m, 4H), 8.24 (s, 1H), 8.91 (s, 1H).

Step 3: To a solution of 1-((trans)-4-(2-((tert-butyldiphenylsilyl)oxy)ethyl)cyclohexyl)-4-nitro-1H-pyrazole (329 mg, 0.69 mmol, 1 eq.) in ethanol (8 mL, 0.08 M) under nitrogen was added Pd/C (110 mg, 0.1 mmol, 0.15 eq.). The atmosphere of the flask was replaced with hydrogen (5 purges). The reaction mixture was stirred under 1 atm of hydrogen at room temperature for 3 h. The reaction mixture was filtered through a celite pad and rinsed with EtOAc (3×20 mL). The solution was concentrated to afford 1-((trans)-4-(2-((tert-butyldiphenylsilyl)oxy)ethyl)cyclohexyl)-1H-pyrazol-4-amine Int. F-1a (339 mg, quantitative yield) as a purple oil, which was used without further purification.

LCMS: [M+H]⁺=448.2.

¹H NMR (400 MHz, DMSO-d₆) δ ppm 1.00 (s, 9H), 1.01-1.05 (m, 1H), 1.38-1.50 (m, 3H), 1.50-1.63 (m, 2H), 1.71 (br d, J=12.0 Hz, 2H), 1.84-1.94 (m, 2H), 3.69 (t, J=6.2 Hz, 2H), 3.73-3.89 (m, 2H), 6.87 (d, J=0.7 Hz, 1H), 7.01 (d, J=0.7 Hz, 1H), 7.41-7.49 (m, 6H), 7.57-7.66 (m, 4H).

Step 2′: Diisopropyl azodicarboxylate (0.46 mL, 2.35 mmol) was slowly added to a solution of triphenylphosphine (617 mg, 2.35 mmol), (trans)-4-(2-((tert-butyldiphenylsilyl)oxy)ethyl)cyclohexan-1-ol trans (600 mg, 1.57 mmol, 1.0 eq.) and 4-nitro-1H-pyrazole (266 mg, 2.35 mmol, 1.5 eq.) in dry THE (6 mL, 0.2 M) at 0° C. The reaction was brought to room temperature and stirred for 18 h. The solvent was evaporated, and the residue was then purified by reverse phase column chromatography (5% to 100% MeOH in water w/0.1% formic acid). The fractions were combined and concentrated to afford 1-((cis)-4-(2-((tert-butyldiphenylsilyl)oxy)ethyl)cyclohexyl)-4-nitro-1H-pyrazole (520 mg, 1.087 mmol, 69% yield) as a yellow solid.

LCMS: [M+H]⁺=478.2.

¹H NMR (400 MHz, DMSO-d₆) δ ppm 0.99 (s, 9H), 1.36-1.48 (m, 2H), 1.50-1.56 (m, 2H), 1.59 (q, J=6.8 Hz, 2H), 1.72-1.86 (m, 3H), 1.96-2.06 (m, 2H), 3.68 (t, J=6.4 Hz, 2H), 4.21-4.35 (m, 1H), 7.39-7.50 (m, 6H), 7.57-7.66 (m, 4H), 8.26 (d, J=0.5 Hz, 1H), 8.93 (s, 1H).

Step 3′: Nitrogen was bubbled for 5 minutes through a solution of 1-((cis)-4-(2-((tert-butyldiphenylsilyl)oxy)ethyl)cyclohexyl)-4-nitro-1H-pyrazole (520 mg, 1.09 mmol, 1.0 eq.) in methanol (2.18 mL, 0.5 M). Pd/C 10% (232 mg, 0.22 mmol, 0.2 eq.) was then added and nitrogen was bubbled for another 5 min. H₂ (1 atm) was then bubbled through the solution for 5 min and the reaction mixture was stirred at room temperature under a hydrogen atmosphere for 18 h. The reaction mixture was filtered through a pad of celite and washed with EtOAc. The residue was concentrated under vacuum to afford 1-((cis)-4-(2-((tert-butyldiphenylsilyl)oxy)ethyl)cyclohexyl)-1H-pyrazol-4-amine Int. F-1b (465 mg, 1.039 mmol, 95% yield) as a purple oil, which was used in the next step without further purification.

LCMS: [M+H]⁺=448.4.

¹H NMR (400 MHz, DMSO-d₆) δ ppm 0.99 (s, 9H), 1.32-1.44 (m, 2H), 1.44-1.51 (m, 2H), 1.55 (q, J=6.8 Hz, 2H), 1.60-1.70 (m, 2H), 1.70-1.79 (m, 1H), 1.83-1.96 (m, 2H), 3.68 (t, J=6.5 Hz, 2H), 3.72-3.80 (m, 1H), 3.90-4.00 (m, 1H), 6.88 (s, 1H), 7.04 (s, 1H), 7.39-7.50 (m, 6H), 7.57-7.66 (m, 4H). One proton was not observed in the ¹H NMR spectrum.

TABLE 24
List of Int. F-2 compounds generated by Representative Procedure TBM-F: Step 1

| Starting | | | | | |
|---|---|---|---|---|---|
| Material | | Product | | | |
| Int. | Int. | Int. | | | |
| F-1 | D-1 | F-2 | Structure | Characterization | Notes |
| Int. F-1a | Int. D-1a | Int. F-2a | 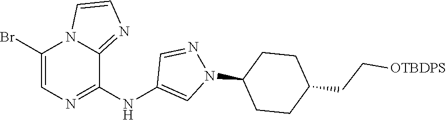 | 67% yield, light-orange solid [M + H]⁺ = 643.2, 645.2. ¹H NMR (400 MHz, DMSO-d6) δ ppm 1.01 (s, 9 H), 1.03-1.13 (m, 2 H), 1.42-1.53 (m, 3 H), 1.60- 1.72 (m, 2 H), 1.72- 1.80 (m, 2 H), 1.94-2.02 (m, 2 H), 3.71 (br t, J = 5.9 Hz, 2 H), 4.00-4.08 (m, 1 H), 7.42-7.50 (m, 6 H), 7.59 (s, 1 H), 7.60- 7.65 (m, 4 H), 7.70 (d, J = 1.0 Hz, 1 H), 7.73 (s, 1 H), 7.98 (d, J = 1.0 Hz, 1 H), 8.13 (s, 1 H), 9.97 (s, 1 H). | |
| Int. F-1b | Int. D-1a | Int. F-2b | 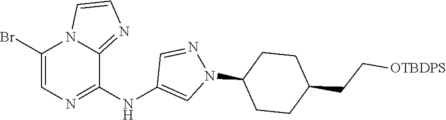 | 71% yield, white solid [M + H]⁺ = 643.2. ¹H NMR (400 MHz, DMSO-d6) δ ppm 1.00 (s, 9 H), 1.38-1.50 (m, 2 H), 1.50-1.63 (m, 4 H), 1.70- 1.84 (m, 3 H), 1.88- 2.02 (m, 2 H), 3.70 (t, J = 6.5 Hz, 2 H), 4.07-4.25 (m, 1 H), 7.40-7.49 (m, 6 H), 7.59 (s, 1 H), 7.60- 7.66 (m, 4 H), 7.70 (d, J = 1.0 Hz, 1 H), 7.75 (s, 1 H), 7.98 (d, J = 1.2 Hz, 1 H), 8.16 (s, 1 H), 9.98 (s, 1 H). | |
| Int. F-1b | Int. D-1b | Int. F-2c | 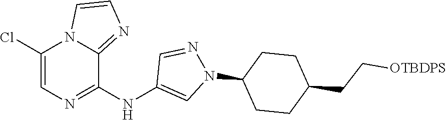 | 68% yield yellow semi- solid [M + H]⁺ = 598.2 ¹H NMR (400 MHz, CHLOROFORM-d) 8 ppm 1.06 (s, 9 H), 1.48- 1.57 (m, 2 H), 1.57-1.68 (m, 4 H), 1.83-1.96 (m, 3 H), 1.99-2.10 (m, 2 H), 3.72 (t, J = 6.6 Hz, 2 H), 4.11-4.21 (m, 1 H), 6.33- 6.41 (m, 1 H), 6.64 (s, 1 H), 6.71 (d, J = 7.8 Hz, 1 H), 7.35-7.45 (m, 6 H), 7.46-7.49 (m, 1 H), 7.52- 7.55 (m, 1 H), 7.58- 7.62 (m, 1 H), 7.64-7.71 (m, 4 H), 7.71-7.75 (m, 1 H). | Buchwald- Hartwig Coupling: Pd(OAc)₂, rac- BINAP, Cs₂CO₃, toluene, 120° C., 4 h |

### Synthesis of TBM-F: Step 2

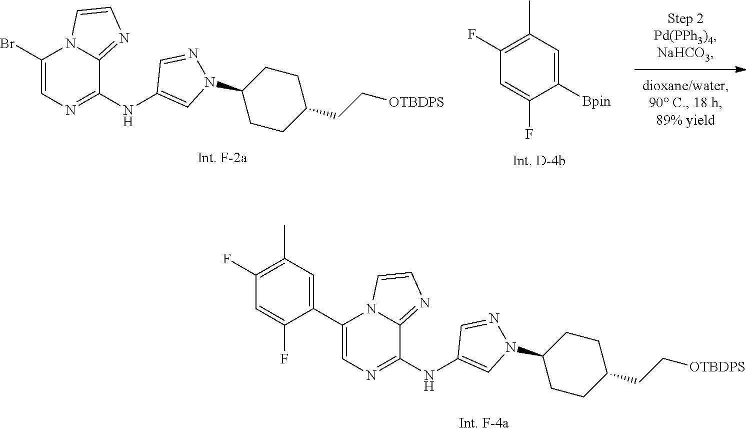

To a solution of 2-(2,4-difluoro-5-methyl-phenyl)-4,4,5,5-tetramethyl-1,3,2-dioxaborolane Int. D-4b (0.69 g, 2.54 mmol, 1.5 eq.) and 5-bromo-N-(1-((trans)-4-(2-((tert-butyldiphenylsilyl)oxy)ethyl)cyclohexyl)-1H-pyrazol-4-yl)imidazo[1,2-a]pyrazin-8-amine Int. F-2a (1.09 g, 1.69 mmol,) in 1,4-dioxane (15 mL) was added NaHCO₃ (0.43 g, 5.1 mmol, 3 eq.) and water (5 mL). Nitrogen was bubbled into the reaction mixture for 10 min. Then, Pd(PPh₃)₄ (0.2 g, 0.17 mmol, 0.1 eq.) was added and nitrogen was bubbled for an additional 10 min. The resulting solution was stirred at 90° C. in a sealed tube under a nitrogen atmosphere for 18 h. The reaction mixture was filtered over Celite, rinsed with EtOAc and concentrated. The resulting residue was purified by reverse phase column chromatography (5% to 100% MeOH in water w/0.1% formic acid) The fractions were combined and concentrated to afford N-(1-((trans)-4-(2-((tert-butyldiphenylsilyl)oxy)ethyl)cyclohexyl)-1H-pyrazol-4-yl)-5-(2,4-difluoro-5-methylphenyl)imidazo[1,2-a]pyrazin-8-amine Int. F-4a (1.07 g, 89% yield) as a light-orange oil.

LCMS: [M-TBDPS+H]⁺=453.2.

¹H NMR (400 MHz, DMSO-d₆) δ ppm 1.00-1.03 (m, 9H), 1.04-1.18 (m, 2H), 1.45-1.53 (m, 3H), 1.61-1.80 (m, 4H), 1.95-2.04 (m, 2H), 2.29 (s, 3H), 3.72 (br t, J=6.0 Hz, 2H), 4.02-4.13 (m, 1H), 7.41-7.49 (m, 8H), 7.58-7.65 (m, 7H), 7.78 (s, 1H), 8.19 (s, 1H), 9.96 (s, 1H).

¹⁹F NMR (377 MHz, DMSO-d₆) δ ppm −112.22-−112.10 (m, 1 F), −111.33-−111.21 (m, 1 F).

TABLE 25
List of intermediates used in TBM-F: Step 2
Starting Material

| Int. | Structure | Source |
|---|---|---|
| Int. F-3 | 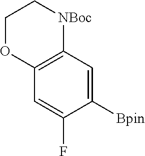 | Synthesis provided below |

### Synthesis of tert-butyl 7-fluoro-6-(4,4,5,5-tetramethyl-1,3,2-dioxaborolan-2-yl)-2,3-dihydro-4H-benzo[b][1,4]oxazine-4-carboxylate (Int. F-3)

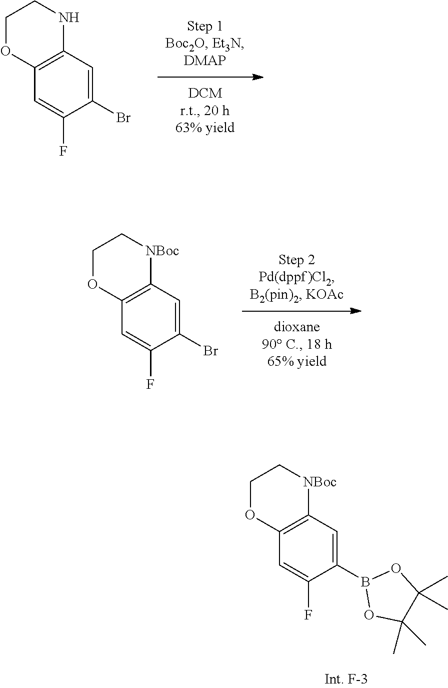 [SMILES: Fc1cc2c(cc1Br)NCCO2 ; CC(C)(C)OC(=O)N1CCOc2cc(F)c(Br)cc21 ; CC(C)(C)OC(=O)N1CCOc2cc(F)c(B3OC(C)(C)C(C)(C)O3)cc21]

Step 1: To a solution of 6-bromo-7-fluoro-3,4-dihydro-2H-1,4-benzoxazine (200 mg, 0.86 mmol, 1.0 eq.) in CH₂Cl₂ (2 mL, 0.4 M) was added tert-butoxycarbonyl tert-butyl carbonate (207 mg, 0.95 mmol, 1.1 eq.) and Et₃N (0.26 mL, 1.9 mmol, 2.2 eq.). The resulting solution was stirred at r.t. for 2 h. LCMS showed traces of desired product. DMAP (105 mg, 0.86 mmol, 1.0 eq.) was added and the reaction mixture was stirred overnight. The reaction mixture was diluted with CH₂Cl₂ and saturated NaHCO₃ and extracted with CH₂Cl₂. The organic layers were then washed with saturated NaHCO₃, dried over sodium sulfate, filtered, and concentrated. The crude residue was purified by reverse phase column chromatography (5% to 100% MeOH in water w/ 0.1% formic acid). The fractions were combined and concentrated to afford tert-butyl 6-bromo-7-fluoro-2,3-dihydro-4H-benzo[b][1,4]oxazine-4-carboxylate (180 mg, 63% yield) as a white solid.

LCMS: [M-Boc+H]⁺=232.0.

¹H NMR (400 MHz, DMSO-d₆) δ ppm 1.49 (s, 9H), 3.78 (t, J=4.5 Hz, 2H), 4.24 (t, J=4.5 Hz, 2H), 6.99 (d, J=9.8 Hz, 1H), 8.08 (br s, 1H).

¹⁹F NMR (377 MHz, DMSO-d₆) δ ppm −113.57 (s, 1 F).

Step 2: To a mixture of tert-butyl 6-bromo-7-fluoro-2,3-dihydro-4H-benzo[b][1,4]oxazine-4-carboxylate (190 mg, 0.57 mmol, 1.0 eq.), B₂pin₂ (218 mg, 0.86 mmol, 1.5 eq.) and KOAc (168 mg, 1.72 mmol, 3.0 eq.) in 1,4-Dioxane (2.3 mL, 0.25 M) was sparged with nitrogen for 10 min. Then, Pd(dppf)Cl₂·CH₂Cl₂ (47 mg, 0.06 mmol, 0.1 eq.) was added and nitrogen was bubbled for an additional 5 min. The resulting solution was stirred at 90° C. in a sealed tube, overnight. The reaction mixture was filtered over Celite, rinsed with EtOAc and concentrated. The resulting residue was purified by silica gel column chromatography (0 to 30% EtOAc in heptane). The fractions were combined and concentrated to afford tert-butyl 7-fluoro-6-(4,4,5,5-tetramethyl-1,3,2-dioxaborolan-2-yl)-2,3-dihydro-4H-benzo[b][1,4]oxazine-4-carboxylate Int. F-3 (170 mg, 65% yield) as a light yellow solid.

LCMS: [M+H]⁺=380.2.

¹H NMR (400 MHz, DMSO-d₆) δ ppm 1.27 (s, 12H), 1.49 (s, 9H), 3.78 (t, J=4.3 Hz, 2H), 4.25 (t, J=4.4 Hz, 2H), 6.67 (d, J=9.8 Hz, 1H), 8.04 (br d, J=5.1 Hz, 1H).

¹⁹F NMR (377 MHz, DMSO-d₆) δ ppm −107.09 (s, 1 F).

TABLE 26
List of Int. F-4 compounds generated by Representative Procedure TBM-F: Step 2

| Starting | | | | | |
|---|---|---|---|---|---|
| Material | | Product | | | |
| Int. | | Int. | | | |
| F-2 | Int. | F-4 | Structure | Characterization | Notes |
| Int. F- 2a | Int. D-4b | Int. F-4a | 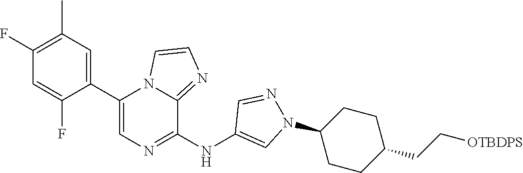 | 89% yield, light- orange oil [M − TBDPS + H]⁺ = 453.2 ¹H NMR (400 MHz, DMSO-d₆) δ ppm 1.00-1.03 (m, 9 H), 1.04-1.18 (m, 2 H), 1.45-1.53 (m, 3 H), 1.61-1.80 (m, 4 H), 1.95-2.04 (m, 2 H), 2.29 (s, 3 H), 3.72 (br t, J = 6.0 Hz, 2 H), 4.02-4.13 (m, 1 H), 7.41-7.49 (m, 8 H), 7.58-7.65 (m, 7 H), 7.78 (s, 1 H), 8.19 (s, 1 H), 9.96 (s, 1 H). ¹⁹F NMR (377 MHz, DMSO-d₆) δ ppm −112.22-−112.10 (m, 1 F), −111.33- −111.21 (m, 1 F). | Conditions: Pd(PPh₃)₄, NaHCO₃, dioxane/ water 90° C., 18 h |
| Int. F- 2b | Int. D-4b | Int. F-4b | 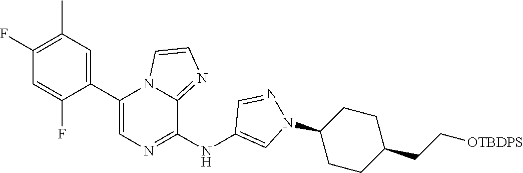 | 62% yield, yellow oil [M + H]⁺ = 691.4. ¹H NMR (400 MHz, DMSO-d₆) δ ppm 1.00 (s, 9 H), 1.38- 1.51 (m, 2 H), 1.51- 1.63 (m, 4 H), 1.70- 1.84 (m, 3 H), 1.91- 2.04 (m, 2 H), 2.28 (s, 3 H), 3.70 (t, J = 6.5 Hz, 2 H), 4.11- 4.23 (m, 1 H), 7.45 (br d, J = 1.2 Hz, 8 H), 7.62 (br s, 7 H), 7.79 (s, 1 H), 8.23 (s, 1 H), 9.97 (s, 1 H). ¹⁹F NMR (377 MHz, DMSO-d₆) δ ppm − 112.15 (q, J = 8.6 Hz, 1 F), −111.28 (q, J = 8.2 Hz, 1 F). | Conditions: Pd(PPh₃)₄, NaHCO₃, dioxane/ water 90° C., 18 h |
| Int. F- 2a | Int. D-4g | Int. F-4c | 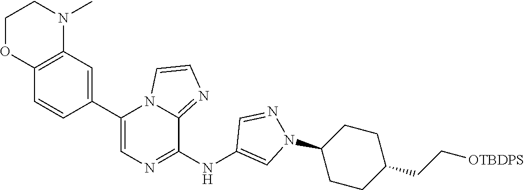 | 60% yield, yellow oil ¹H NMR (400 MHz, DMSO-d₆) δ ppm 0.95-1.04 (m, 9 H), 1.05-1.18 (m, 3 H), 1.44-1.54 (m, 3 H), 1.56-1.83 (m, 4 H), 1.60-1.81 (m, 1 H), 1.96-2.04 (m, 2 H), 2.89 (s, 3 H), 3.72 (br t, J = 5.5 Hz, 2 H), 4.01-4.07 (m, 1 H), 4.30 (br d, J = 4.2 Hz, 2 H), 6.81-6.93 (m, 3 H), 7.37 (s, 1 H), 7.41-7.50 (m, 6 H), 7.59-7.66 (m, 5 H), 7.76 (s, 1 H), 7.88 (s, 1 H), 9.77 (s, 1 H). | Conditions: Xphos-Pd- G3 Xphos, dioxane/ water 90° C., 18 h |
| Int. F- 2b | Int. D-4a | Int. F-4d | 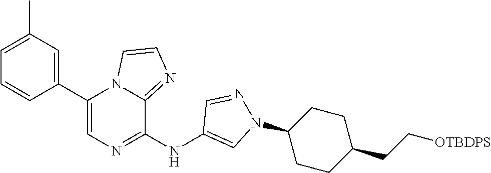 | 91% yield, light- orange solid ¹H NMR (400 MHz, DMSO-d₆) δ ppm 1.00 (s, 9 H), 1.21- 1.27 (m, 1 H), 1.41- 1.51 (m, 2 H), 1.52- 1.63 (m, 4 H), 1.72- 1.84 (m, 3 H), 1.92- 1.98 (m, 1 H), 2.40 (s, 3 H), 3.70 (t, J = 6.5 Hz, 2 H), 4.13- 4.20 (m, 1 H), 7.30- 7.34 (m, 1 H), 7.40- 7.48 (m, 9 H), 7.49- 7.51 (m, 1 H), 7.58- 7.66 (m, 5 H), 7.79 (s, 1 H), 7.89 (s, 1 H), 8.23 (s, 1 H), 9.88 (s, 1 H). | Conditions: Pd(PPh₃)₄, NaHCO₃, dioxane/ water 90° C., 18 h |
| Int. F- 2a | Int. D-4d | Int. F-4e | 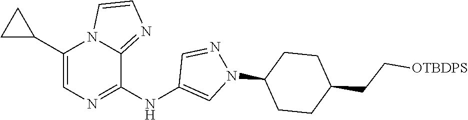 | 74% yield, off-white solid [M + H]⁺ = 605.3 ¹H NMR (400 MHz, DMSO-d₆) δ ppm 0.68-0.76 (m, 2 H), 0.95-1.01 (m, 3 H), 1.01 (s, 9 H), 1.05- 1.13 (m, 2 H), 1.48 (br s, 4 H), 1.65 (qd, J = 12.5, 3.4 Hz, 2 H), 1.75 (br d, J = 12.0 Hz, 2 H), 1.94-2.08 (m, 3 H), 3.71 (t, J = 6.1 Hz, 2 H), 4.04 (s, 1 H), 7.22 (d, J = 1.2 Hz, 1 H), 7.46 (m, 6 H), 7.60-7.66 (m, 5 H), 7.71 (s, 1 H), 8.05 (d, J = 1.0 Hz, 1 H), 8.13 (s, 1 H), 9.62 (s, 1 H). | Kumada Coupling: Pd(dppf)Cl₂, THF, 80° C., 1 h |
| Int. F- 2a | Int. D-4a | Int. F-4f | 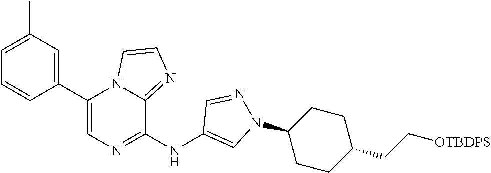 | 78% yield, yellow solid [M − TBDPS + H]⁺ = 417.2 | Conditions: Xphos-Pd- G3 Xphos, dioxane/ water 90° C., 18 h |
| Int. F- 2a | Int. F-3 | Int. F-4g | 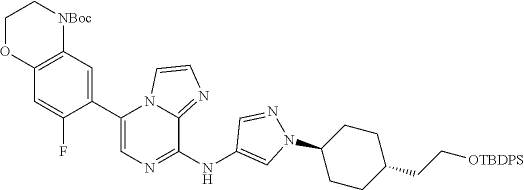 | 55% yield, light- green semi-solid [M − TBDPS + H]⁺ = 579.0 | Conditions: Pd(PPh₃)₄, NaHCO₃, dioxane/ water 90° C., 18 h |
| Int. F- 2c | Int. D-4b | Int. F-4h | 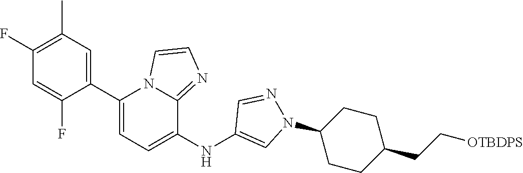 | 67% yield, tan solid [M + H]⁺ = 690.2 ¹H NMR (400 MHz, CHLOROFORM-d) 8 ppm 1.06 (s, 9 H), 1.24-1.30 (m, 1 H), 1.46-1.58 (m, 2 H), 1.59-1.65 (m, 5 H), 1.79-1.98 (m, 3 H), 2.00-2.12 (m, 2 H), 2.31 (s, 3 H), 3.72 (t, J = 6.5 Hz, 2 H), 4.11- 4.21 (m, 1 H), 6.47 (d, J = 7.6 Hz, 1 H), 6.64 (d, J = 7.6 Hz, 1 H), 6.77 (br s, 1 H), 6.95 (t, J = 9.4 Hz, 1 H), 7.29-7.35 (m, 2 H), 7.35-7.46 (m, 6 H), 7.50-7.55 (m, 2 H), 7.58 (s, 1 H), 7.65- 7.71 (m, 4 H). ¹⁹F NMR (377 MHz, CHLOROFORM-d) 8 ppm −112.04- −111.81 (m, 1 F), −111.26-−111.00 (m, 1 F). | Conditions: Pd(PPh₃)₄, Cs₂CO₃, dioxane/ water 110° C., 16 h |

### Synthesis of TBM-F: Step 3

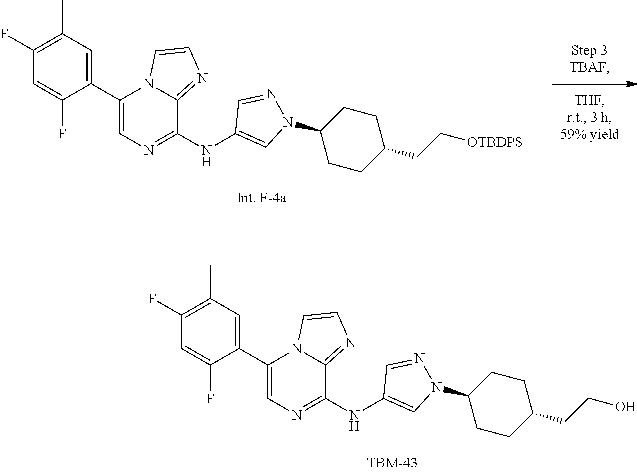

To a stirred solution of N-(1-((trans)-4-(2-((tert-butyldiphenylsilyl)oxy)ethyl)cyclohexyl)-1H-pyrazol-4-yl)-5-(2,4-difluoro-5-methylphenyl)imidazo[1,2-a]pyrazin-8-amine Int. F-4a (1.07 g, 1.55 mmol, 1 eq.) in THF (16 mL) was added 1 M TBAF solution in THF (3.5 mL, 3.5 mmol, 2.2 eq.) at room temperature for 3 h. The solvents were removed under reduced pressure and the residue was purified by silica gel column chromatography (0% to 30% MeOH in DCM). The fractions were combined, concentrated and the residue was then purified by reverse phase column chromatography (5% MeOH to 100% MeOH in water w/ 0.1% formic acid). The fractions were combined and concentrated to afford 2-((trans)-4-(4-((5-(2,4-difluoro-5-methylphenyl)imidazo[1,2-a]pyrazin-8-yl)amino)-1H-pyrazol-1-yl)cyclohexyl)ethan-1-ol TBM-43 (425 mg, 59% yield) as a light yellow solid.

LCMS: [M+H]⁺=453.2.

¹H NMR (400 MHz, DMSO-d₆) δ ppm 1.04-1.17 (m, 2H), 1.37 (q, J=6.5 Hz, 2H), 1.41-1.50 (m, 1H), 1.71 (qd, J=12.5, 2.9 Hz, 2H), 1.80-1.88 (m, 2H), 1.99-2.07 (m, 2H), 2.27-2.30 (m, 3H), 3.43-3.49 (m, 2H), 4.04-4.14 (m, 1H), 4.31-4.38 (m, 1H), 7.40-7.48 (m, 2H), 7.58-7.64 (m, 3H), 7.78 (s, 1H), 8.20 (s, 1H), 9.96 (s, 1H).

TABLE 27
List of TBM-F compounds generated by Representative Procedure TBM-F: Step 3

| Starting | | | |
|---|---|---|---|
| Material | Product | | |
| Int. F-4 | TBM | Structure | Characterization |
| Int. F-4a | TBM-43 | 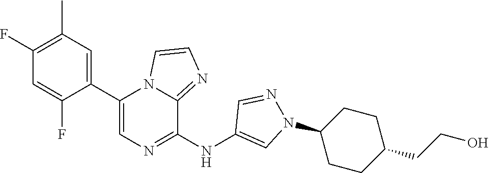 [SMILES: Cc1cc(-c2cnc(Nc3cnn([C@H]4CC[C@H](CCO)CC4)c3)c3nccn23)c(F)cc1F] | 59% yield, light-yellow solid [M + H]⁺ = 453.2 ¹H NMR (400 MHz, DMSO-d6) δ ppm 1.04-1.17 (m, 2 H), 1.37 (q, J = 6.5 Hz, 2 H), 1.41-1.50 (m, 1 H), 1.71 (qd, J = 12.5, 2.9 Hz, 2 H), 1.80-1.88 (m, 2 H), 1.99-2.07 (m, 2 H), 2.27-2.30 (m, 3 H), 3.43-3.49 (m, 2 H), 4.04-4.14 (m, 1 H), 4.31-4.38 (m, 1 H), 7.40-7.48 (m, 2 H), 7.58-7.64 (m, 3 H), 7.78 (s, 1 H), 8.20 (s, 1 H), 9.96 (s, 1 H). |
| Int. F-4b | TBM-44 | 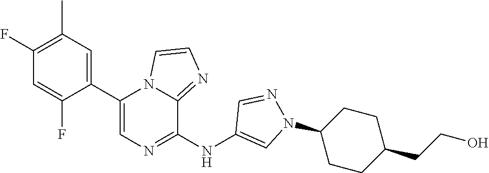 [SMILES: Cc1cc(-c2cnc(Nc3cnn([C@H]4CC[C@@H](CCO)CC4)c3)c3nccn23)c(F)cc1F] | 89% yield, light-yellow solid [M + H]⁺ = 435.4 ¹H NMR (400 MHz, DMSO-d₆) δ ppm 1.41-1.53 (m, 4 H), 1.54- 1.66 (m, 2 H), 1.67-1.87 (m, 3 H), 1.98-2.06 (m, 2 H), 2.29 (s, 3 H), 3.40-3.48 (m, 2 H), 4.13-4.25 (m, 1 H), 4.34 (t, J = 5.0 Hz, 1 H), 7.41-7.48 (m, 2 H), 7.57-7.65 (m, 3 H), 7.80 (s, 1 H), 8.24 (s, 1 H), 9.97 (s, 1 H). ¹⁹F NMR (377 MHz, DMSO-d₆) δ ppm −112.12 (q, J = 8.6 Hz, 1 F), −111.29 (q, J = 8.2 Hz, 1 F). |
| Int. F-4c | TBM-45 | 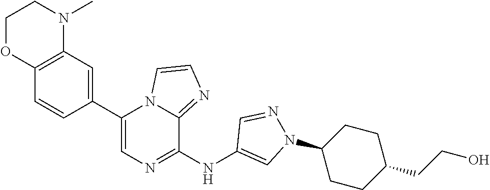 [SMILES: CN1CCOc2ccc(-c3cnc(Nc4cnn([C@H]5CC[C@H](CCO)CC5)c4)c4nccn34)cc21] | 64% yield, light-yellow oil [M + H]⁺ = 474.4 ¹H NMR (400 MHz, DMSO-d₆) δ ppm 1.03-1.15 (m, 2 H), 1.33- 1.50 (m, 3 H), 1.63-1.77 (m, 2 H), 1.79-1.88 (m, 2 H), 2.00- 2.07 (m, 2 H), 2.89 (s, 3 H), 3.46 (br t, J = 6.5 Hz, 2 H), 4.06- 4.14 (m, 1 H), 4.24-4.40 (m, 3 H), 6.81-6.93 (m, 3 H), 7.37 (s, 1 H), 7.61 (s, 1 H), 7.76 (s, 1 H), 7.86-7.90 (m, 1 H), 8.13 (s, 1 H), 8.20 (s, 1 H), 9.77 (s, 1 H), 12.46-13.02 (m, 1 H). |
| Int. F-4d | TBM-46 | 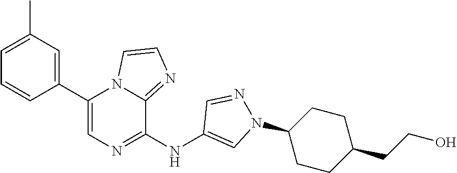 [SMILES: Cc1cccc(-c2cnc(Nc3cnn([C@H]4CC[C@@H](CCO)CC4)c3)c3nccn23)c1] | 84% yield, light-yellow oil [M + H]⁺ = 417.2 ¹H NMR (400 MHz, DMSO-d₆) δ ppm 1.46-1.53 (m, 3 H), 1.55- 1.65 (m, 2 H), 1.69-1.75 (m, 1 H), 1.76-1.85 (m, 2 H), 1.99- 2.10 (m, 2 H), 2.41 (s, 3 H), 3.44 (q, J = 6.0 Hz, 2 H), 4.15- 4.23 (m, 1 H), 4.35 (t, J = 5.1 Hz, 1 H), 7.32 (br d, J = 6.8 Hz, 1 H), 7.42-7.51 (m, 4 H), 7.62- 7.66 (m, 1 H), 7.79 (s, 1 H), 7.89 (d, J = 1.0 Hz, 1 H), 8.25 (s, 1 H), 9.87 (s, 1 H), 12.73 (s, 1 H). |
| Int. F-4e | TBM-47 | 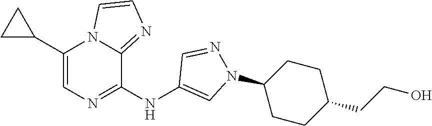 [SMILES: OCC[C@H]1CC[C@H](n2cc(Nc3ncc(C4CC4)n4ccnc34)cn2)CC1] | 89% yield, yellow solid [M + H]⁺ = 367.4 ¹H NMR (400 MHz, DMSO-d₆) δ ppm 0.68-0.78 (m, 2 H), 0.95- 1.03 (m, 2 H), 1.09 (qd, J = 13.2, 3.2 Hz, 2 H), 1.31-1.40 (m, 2 H), 1.41-1.52 (m, 1 H), 1.68 (qd, J = 12.5, 3.3 Hz, 2 H), 1.83 (br d, J = 12.0 Hz, 2 H), 1.97-2.07 (m, 3 H), 3.45 (br s, 2 H), 4.05 (tt, J = 11.8, 3.8 Hz, 1 H), 4.34 (br s, 1 H), 7.21 (d, J = 1.2 Hz, 1 H), 7.65 (d, J = 1.2 Hz, 1 H), 7.72 (s, 1 H), 8.04 (d, J = 1.0 Hz, 1 H), 8.14 (s, 1 H), 9.63 (s, 1 H). |
| Int. F-4f | TBM-48 | 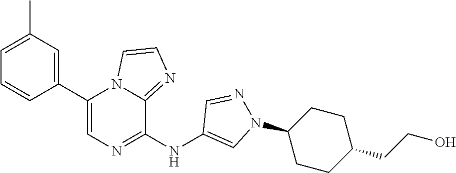 [SMILES: Cc1cccc(-c2cnc(Nc3cnn([C@H]4CC[C@H](CCO)CC4)c3)c3nccn23)c1] | 66% yield, yellow oil [M + H]⁺ = 417.2 |
| Int. F-4g | TBM-49 | 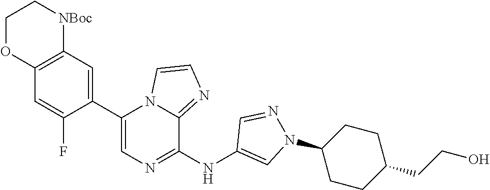 [SMILES: CC(C)(C)OC(=O)N1CCOc2cc(F)c(-c3cnc(Nc4cnn([C@H]5CC[C@H](CCO)CC5)c4)c4nccn34)cc21] | 87% yield, light-yellow semi- solid [M + H]⁺ = 578.2. |
| Int. F-4h | TBM-50 | 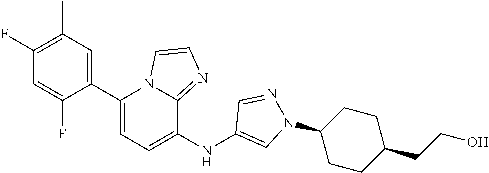 [SMILES: Cc1cc(-c2ccc(Nc3cnn([C@H]4CC[C@@H](CCO)CC4)c3)c3nccn23)c(F)cc1F] | 76% yield, red semi-solid ¹H NMR (400 MHz, CHLOROFORM-d) δ ppm 1.55- 1.79 (m, 7 H), 1.79-1.88 (m, 2 H), 2.14-2.25 (m, 2 H), 2.31 (s, 3 H), 3.69-3.78 (m, 2 H), 4.17-4.27 (m, 1 H), 6.50 (d, J = 7.6 Hz, 1 H), 6.66 (d, J = 7.8 Hz, 1 H), 6.95 (t, J = 9.4 Hz, 1 H), 7.05 (s, 1 H), 7.28-7.36 (m, 2 H), 7.51-7.57 (m, 2 H), 7.60 (s, 1 H). ¹⁹F NMR (377 MHz, CHLOROFORM-d) δ ppm −111.98-−111.80 (m, 1 F), −111.20-−110.90 (m, 1 F). |

### Example S16. General Procedure for TBM-G

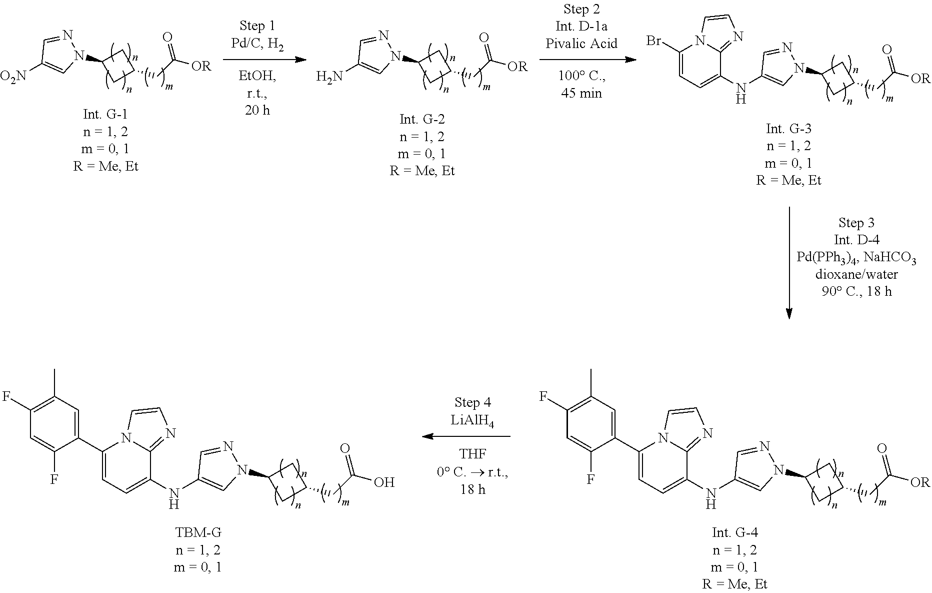

### Synthesis of TBM-G: Step 1

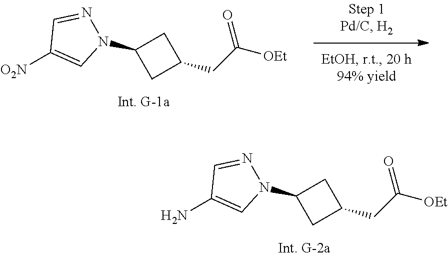 [SMILES: CCOC(=O)C[C@H]1C[C@H](n2cc([N+](=O)[O-])cn2)C1 ; CCOC(=O)C[C@H]1C[C@H](n2cc(N)cn2)C1]

To a solution of ethyl 2-((trans)-3-(4-nitro-1H-pyrazol-1-yl)cyclobutyl)acetate Int. G-1a (414 mg, 1.6 mmol, 1 eq.) in ethanol (8 mL, 0.2 M) under nitrogen was added Pd/C (261 mg, 0.25 mmol, 0.15 eq.). The atmosphere of the flask was replaced with hydrogen (5 purges). The reaction mixture was stirred under 1 atm of hydrogen at room temperature for 20 h. The reaction mixture was filtered through a Celite pad and rinsed with EtOAc (3×50 mL). The resulting filtrate was concentrated to afford ethyl 2-((trans)-3-(4-amino-1H-pyrazol-1-yl)cyclobutyl)acetate Int. G-2a (391 mg, 94% yield) as a purple oil, which was used without further purification.

LCMS: [M+H]⁺=224.2.

¹H NMR (400 MHz, DMSO-d₆) δ ppm 1.15-1.23 (m, 4H), 2.05-2.16 (m, 2H), 2.52-2.59 (m, 4H), 3.79 (br s, 2H), 4.05 (q, J=7.1 Hz, 2H), 4.76 (quint, J=7.6 Hz, 1H), 6.93 (s, 1H), 7.07 (s, 1H).

TABLE 28
List of intermediate used in TBM-G: Step 1
Starting Material

| Int. G-1 | Structure | Source |
|---|---|---|
| Int. G-1a | 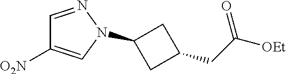 [SMILES: CCOC(=O)C[C@H]1C[C@H](n2cc([N+](=O)[O-])cn2)C1] | Synthesis provided below |
| Int. G-1b | 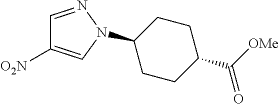 [SMILES: COC(=O)[C@H]1CC[C@H](n2cc([N+](=O)[O-])cn2)CC1] | Synthesis provided below |
| Int. G-1c | 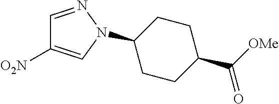 [SMILES: COC(=O)[C@H]1CC[C@@H](n2cc([N+](=O)[O-])cn2)CC1] | Synthesis provided below |
| Int. G-1d | 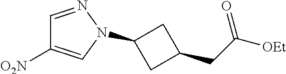 [SMILES: CCOC(=O)C[C@H]1C[C@@H](n2cc([N+](=O)[O-])cn2)C1] | Synthesis provided below |
| Int. G-1e | 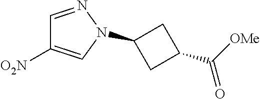 [SMILES: COC(=O)[C@H]1C[C@H](n2cc([N+](=O)[O-])cn2)C1] | Synthesis provided below |

### Synthesis of ethyl 2-((trans)-3-(4-nitro-1H-pyrazol-1-yl)cyclobutyl)acetate (Int. G-1a) and ethyl 2-((cis)-3-(4-nitro-1H-pyrazol-1-yl)cyclobutyl)acetate (Int. G-1d)

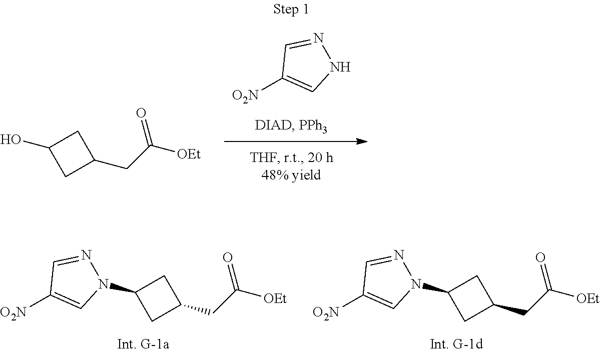 [SMILES: O=[N+]([O-])c1cn[nH]c1 ; CCOC(=O)CC1CC(O)C1 ; CCOC(=O)C[C@H]1C[C@H](n2cc([N+](=O)[O-])cn2)C1 ; CCOC(=O)C[C@H]1C[C@@H](n2cc([N+](=O)[O-])cn2)C1]

To a solution of ethyl 2-(3-hydroxycyclobutyl)acetate (850 mg, 5.37 mmol, 1 eq.), triphenylphosphine polymer bound (3.58 g, 10.75 mmol, 2 eq.) and 4-nitro-1H-pyrazole 2 (1.22 g, 10.75 mmol, 2 eq.) in anhydrous THF (13 mL, 0.2 M) at 0° C. was slowly added diisopropyl azodicarboxylate (2.11 mL, 10.75 mmol, 2 eq.) as a solution in anhydrous THF (13 mL, 0.2 M) and the reaction was warmed to room temperature. After stirring for 20 h under a nitrogen atmosphere, the solvent was removed under reduced pressure and the residue was purified by silica gel column chromatography (0 to 10% EtOAc in DCM). The fractions were combined and concentrated to afford 747 mg of the mixture of cis and trans isomers. The isomers were separated by chiral SFC, affording ethyl 2-((trans)-3-(4-nitro-1H-pyrazol-1-yl)cyclobutyl)acetate Int. G-la (414 mg, 30% yield) as a white solid and ethyl 2-((cis)-3-(4-nitro-1H-pyrazol-1-yl)cyclobutyl)acetate Int. G-1d (239 mg, 18% yield) as a colorless oil. Int. G-1a:

LCMS: [M+H]⁺=254.2.

¹H NMR (400 MHz, CHLOROFORM-d) δ ppm 1.28 (t, J=7.1 Hz, 3H), 2.36-2.45 (m, 2H), 2.59 (d, J=7.6 Hz, 2H), 2.73-2.83 (m, 2H), 2.85-2.97 (m, 1H), 4.16 (q, J=7.1 Hz, 2H), 4.89 (quin, J=7.5 Hz, 1H), 8.12 (s, 1H), 8.18 (s, 1H).

### Synthesis of methyl (trans)-4-(4-nitro-1H-pyrazol-1-yl)cyclohexane-1-carboxylate (Int. G-1b)

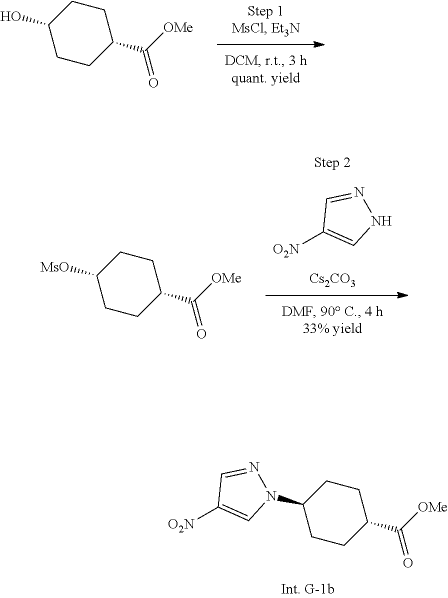

Step 1: To a solution of methyl cis-4-hydroxycyclohexanecarboxylate (3 g, 19 mmol) in CH₂Cl₂ (65 mL) were added Et₃N (7.66 mL, 56.9 mmol) and MsCl (1.91 mL, 24.7 mmol). After 3 h at room temperature, the reaction mixture was quenched with a sat. aq. NH₄Cl and diluted with CH₂Cl₂. The organic phase was separated, dried over Na₂SO₄, filtered, and concentrated in vacuo to afford methyl cis-4-methylsulfonyloxycyclohexanecarboxylate (4.5 g, 19 mmol, quant.) as a light yellow solid, which was used in the next step without further purification.

¹H NMR (400 MHz, CDCl₃) δ ppm 1.67-1.83 (m, 4H), 1.87-1.98 (m, 2H), 1.99-2.09 (m, 2H), 2.35-2.45 (m, 1H), 3.01 (s, 3H), 3.68 (s, 3H), 4.87-4.96 (m, 1H).

Step 2: To a solution of methyl cis-4-methylsulfonyloxycyclohexanecarboxylate (4.26 g, 18 mmol) and 4-nitro-1H-pyrazole (1.7 g, 15.03 mmol) in DMF (20 mL) was added Cs₂CO₃ (6.86 g, 21.05 mmol). After 4 h at 90° C., the reaction mixture was cooled down to room temperature and partitioned between water (10 mL) and EtOAc (10 mL). The aqueous phase was separated and extracted with EtOAc (3×10 mL). The organic phases were combined, washed with water (3×) and brine, dried over Na₂SO₄, filtered, and concentrated in vacuo. The residue was purified by reverse phase column chromatography (5% to 100% MeCN in water w/ 0.1% formic acid) affording methyl (trans)-4-(4-nitro-1H-pyrazol-1-yl)cyclohexane-1-carboxylate (Int. G-1b) (1.24 g, 4.90 mmol, 33%) as a yellow solid.

¹H NMR (400 MHz, CDCl₃) δ ppm 1.60-1.72 (m, 2H), 1.81 (qd, J=12.6, 2.7 Hz, 2H), 2.19-2.33 (m, 4H), 2.37-2.46 (m, 1H), 3.71 (s, 3H), 4.15 (tt, J=11.7, 3.8 Hz, 1H), 8.08 (s, 1H), 8.16 (s, 1H).

### Synthesis of methyl (cis)-4-(4-nitro-1H-pyrazol-1-yl)cyclohexane-1-carboxylate (Int. G-1c)

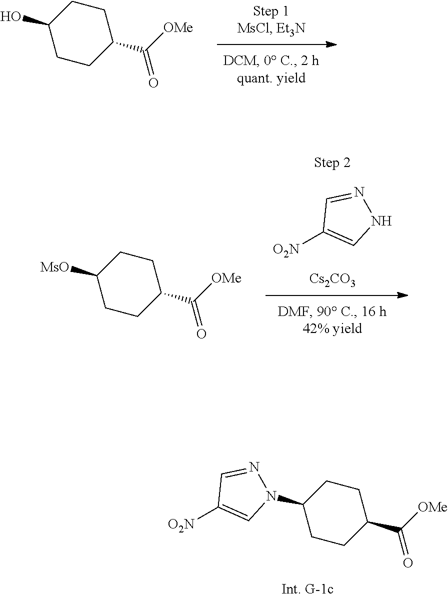

Step 1: Under nitrogen, a solution of methyl trans-4-hydroxycyclohexanecarboxylate (100 mg, 0.63 mmol, 1 eq.) in CH₂Cl₂ (2 mL, 0.32 M) was cooled to 0° C., then methanesulfonyl chloride (64 μL, 0.82 mmol, 1.3 eq.) and triethylamine (0.13 mL, 0.95 mmol, 1.5 eq.) were added. After stirring 2 h at 0° C., the reaction was quenched by the addition of water. The phases were then separated, and the aqueous phase was extracted 3 times with CH₂Cl₂. The combined organic phases were washed twice with brine, dried over magnesium sulfate, filtered, and concentrated to afford methyl trans-4-methylsulfonyloxycyclohexanecarboxylate (150 g, quantitative yield) as a light yellow solid.

¹H NMR (400 MHz, DMSO-d₆) δ ppm 1.43-1.58 (m, 4H), 1.88-2.08 (m, 4H), 2.30-2.41 (m, 1H), 3.17 (s, 3H), 3.59 (s, 3H), 4.53-4.62 (m, 1H).

Step 2: Under nitrogen, in a flame-dried round-bottom flask, a solution of methyl trans-4-methylsulfonyloxycyclohexanecarboxylate (150 mg, 0.64 mmol, 1 eq.), 4-nitro-1H-pyrazole (72 mg, 0.64 mmol, 1 eq.) and DMF (3 mL, 0.21 M) was stirred at room temperature for 5 minutes before Cs₂CO₃ (415 mg, 1.27 mmol, 2.0 eq.) was added. The resulting mixture was stirred at 90° C. for 16 h. The reaction was quenched by the addition of water. Ethyl acetate was added, and the phases were separated. The aqueous phase was extracted 3 times with ethyl acetate, then the combined organic phases were washed once with water and once with brine, dried over magnesium sulfate, filtered, and concentrated. The crude product was purified by reverse phase column chromatography (5% to 100% MeOH in water w/0.1% formic acid). The fractions were combined and concentrated to afford methyl (cis)-4-(4-nitro-1H-pyrazol-1-yl)cyclohexane-1-carboxylate Int. G-lc (68.1 mg, 42% yield) as a tan solid.

LCMS: [M+H]⁺=254.4.

¹H NMR (400 MHz, DMSO-d₆) δ ppm 1.61-1.73 (m, 2H), 1.89-1.96 (m, 4H), 2.00-2.07 (m, 2H), 2.70 (quin, J=4.5 Hz, 1H), 3.64 (s, 3H), 4.25-4.38 (m, 1H), 8.26 (s, 1H), 8.91 (s, 1H).

### Synthesis of methyl (trans)-3-(4-nitro-1H-pyrazol-1-yl)cyclobutane-1-carboxylate (Int. G-1e)

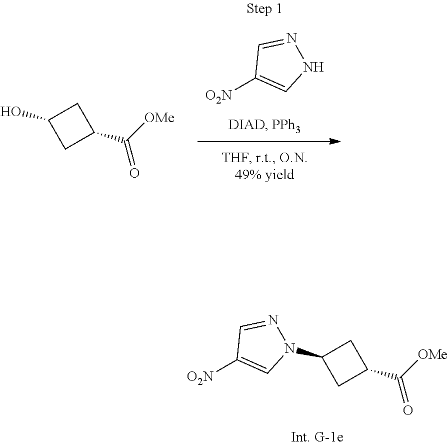

To a solution of 4-nitro-1H-pyrazole (1.04 g, 9.22 mmol), methyl cis-3-hydroxycyclobutanecarboxylate (800 mg, 6.15 mmol) and triphenylphosphine (8.22 g, 12.29 mmol) in dry THF (15 mL) under nitrogen at 0° C. was slowly added a solution of diisopropyl azodicarboxylate (1.82 mL, 9.22 mmol) in THF (5 mL). The following morning, the reaction mixture was filtered, concentrated in vacuo and purified by reverse phase column chromatography (5% to 100% MeCN in water w/ 0.10 formic acid). The fractions were collected and concentrated, affording methyl (trans)-3-(4-nitro-1H-pyrazol-1-yl)cyclobutane-1-carboxylate (Int. G-1e) (963 mg with 300 of triphenylphosphine oxide, corresponding to 675 mg of the desired compound, 49% yield.

LCMS: [M+H]⁺=226.2.

¹H NMR (400 MHz, DMSO-d₆) δ ppm 2.63-2.71 (m, 2H), 2.76-2.85 (m, 2H), 3.19-3.29 (m, 1H), 3.66-3.70 (m, 3H), 5.06-5.16 (i, 1H), 8.34 (s, 1H), 9.01 (s, 1H).

TABLE 29
List of Int. G-2 compounds generated by Representative Procedure TBM-G:
Step 1

| Starting | Product | | |
|---|---|---|---|
| Material | Int. | | |
| Int. G-1 | G-2 | Structure | Characterization |
| Int. G-1a | Int. G-2a | 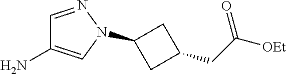 [SMILES: CCOC(=O)C[C@H]1C[C@H](n2cc(N)cn2)C1] | 94% yield, purple oil [M + H]⁺ = 224.2. ¹H NMR (400 MHz, DMSO-d₆) δ ppm 1.15- 1.23 (m, 4 H), 2.05-2.16 (m, 2 H), 2.52-2.59 (m, 4 H), 3.79 (br s, 2 H), 4.05 (q, J = 7.1 Hz, 2 H), 4.76 (quin, J = 7.6 Hz, 1 H), 6.93 (s, 1 H), 7.07 (s, 1 H). |
| Int. G-1b | Int. G-2b | 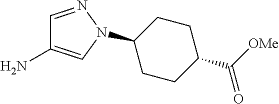 [SMILES: COC(=O)[C@H]1CC[C@H](n2cc(N)cn2)CC1] | quant. yield, yellow semi-solid ¹H NMR (400 MHz, DMSO-d6) δ ppm 1.43- 1.55 (m, 2 H), 1.61-1.73 (m, 2 H), 1.98 (br d, J = 10.1 Hz, 3 H), 2.31-2.40 (m, 1 H), 3.17 (br d, J = 4.3 Hz, 2 H), 3.60 (s, 3 H), 3.87-3.99 (m, 1 H), 4.06-4.13 (m, 1 H), 6.88 (s, 1 H), 7.02 (s, 1 H). |
| Int. G-1c | Int. G-2c | 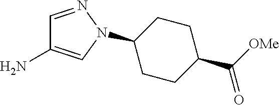 [SMILES: COC(=O)[C@H]1CC[C@@H](n2cc(N)cn2)CC1] | 89% yield, purple oil [M + H]⁺ = 224.2. ¹H NMR (400 MHz, CHLOROFORM-d) δ ppm 1.57 (br s, 2 H), 1.65-1.75 (m, 2 H), 1.89-2.01 (m, 3 H), 2.19-2.25 (m, 1 H), 2.61-2.69 (m, 1 H), 2.80-3.07 (m, 2 H), 3.69-3.73 (m, 3 H), 4.01-4.10 (m, 1 H), 7.03-7.08 (m, J = 0.7 Hz, 1 H), 7.15 (s, 1 H). |
| Int. G-1d | Int. G-2d | 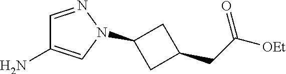 [SMILES: CCOC(=O)C[C@H]1C[C@@H](n2cc(N)cn2)C1] | 99% yield, red oil [M + H]⁺ = 224.2. ¹H NMR (400 MHz, DMSO-d₆) δ ppm 1.18 (t, J = 7.1 Hz, 3 H), 1.98-2.11 (m, 3 H), 2.25-2.36 (m, 2 H), 2.48 (s, 2 H), 3.73-3.84 (m, 2 H), 4.05 (d, J = 7.1 Hz, 2 H), 4.44-4.54 (m, 1 H), 6.90- 6.92 (m, 1 H), 7.08-7.11 (m, 1 H). |
| Int. G-1e | Int. G-2e | 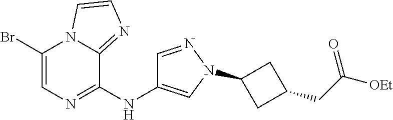 [SMILES: CCOC(=O)C[C@H]1C[C@H](n2cc(Nc3ncc(Br)n4ccnc34)cn2)C1] | 84% yield, red oil [M + H]⁺ = 196.2. ¹H NMR (400 MHz, DMSO-d₆) δ ppm 2.53- 2.58 (m, 2 H), 2.62-2.70 (m, 2 H), 3.08-3.17 (m, 1 H), 3.65-3.67 (m, 3 H), 3.87 (br s, 2 H), 4.74-4.83 (m, 1 H), 6.97 (s, 1 H), 7.11 (s, 1 H). |

### Synthesis of TBM-G: Step 2

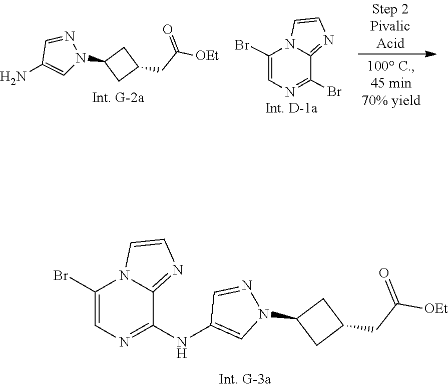 [SMILES: CCOC(=O)C[C@H]1C[C@H](n2cc(N)cn2)C1 ; Brc1ncc(Br)n2ccnc12 ; CCOC(=O)C[C@H]1C[C@H](n2cc(Nc3ncc(Br)n4ccnc34)cn2)C1]

To a round bottom flask was added ethyl 2-((trans)-3-(4-amino-1H-pyrazol-1-yl)cyclobutyl)acetate Int. G-2a (391 mg, 1.75 mmol, 1 eq.), 5,8-dibromoimidazo[1,2-a]pyrazine 5 (485 mg, 1.75 mmol, 1 eq.) and pivalic acid (2.68 g, 26.3 mmol, 15 eq.). The reaction was placed in a pre-heated oil bath at 100° C. for 45 min. The brown residue was quenched with a saturated solution of NaHCO₃ (until pH 7-9) and extracted with MeTHF (3×). The combined organic layers were washed with brine, dried over Na₂SO₄ and concentrated to give a dark red residue, which was then purified by reverse phase column chromatography (5% to 100% MeOH in water w/ 0.1% formic acid). The fractions were combined and concentrated to afford ethyl 2-((trans)-3-(4-((5-bromoimidazo[1,2-a]pyrazin-8-yl)amino)-1H-pyrazol-1-yl)cyclobutyl)acetate Int. G-3a (533 mg, 70% yield) as a brown solid.

LCMS: [M+H]⁺=419.0, 421.0.

¹H NMR (400 MHz, DMSO-d₆) δ ppm 1.19 (t, J=7.1 Hz, 3H), 2.19 (br t, J=8.7 Hz, 2H), 2.56-2.66 (m, 5H), 4.07 (q, J=7.3 Hz, 2H), 4.94-5.03 (m, 1H), 7.59 (s, 1H), 7.70 (d, J=1.0 Hz, 1H), 7.79 (s, 1H), 7.99 (d, J=1.2 Hz, 1H), 8.17 (s, 1H), 10.00 (s, 1H).

TABLE 30
List of Int. G-3 compounds generated by Representative Procedure TBM-G:
Step 2

| Starting | | | | |
|---|---|---|---|---|
| Material | | Product | | |
| Int. | Int. | Int. | | |
| G-2 | D | G-3 | Structure | Characterization |
| Int. G-2a | Int. D-1a | Int. G-3a | 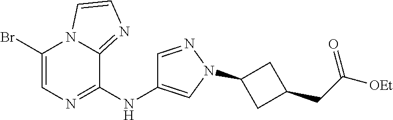 [SMILES: CCOC(=O)C[C@H]1C[C@@H](n2cc(Nc3ncc(Br)n4ccnc34)cn2)C1] | 70% yield, brown solid [M + H]⁺ = 419.0, 421.0. ¹H NMR (400 MHz, DMSO-d₆) δ ppm 1.19 (t, J = 7.1 Hz, 3 H), 2.19 (br t, J = 8.7 Hz, 2 H), 2.56-2.66 (m, 5 H), 4.07 (q, J = 7.3 Hz, 2 H), 4.94-5.03 (m, 1 H), 7.59 (s, 1 H), 7.70 (d, J = 1.0 Hz, 1 H), 7.79 (s, 1 H), 7.99 (d, J = 1.2 Hz, 1 H), |
| Int. G- 2b | Int. D-1a | Int. G- 3b | 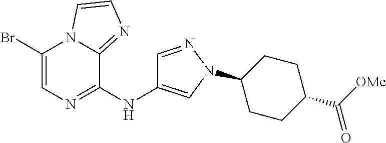 [SMILES: COC(=O)[C@H]1CC[C@H](n2cc(Nc3ncc(Br)n4ccnc34)cn2)CC1] | 8.17 (s, 1 H), 10.00 (s, 1 H). 72% yield, yellow solid [M + H]⁺ = 419.1. |
| Int. G-2c | Int D-1a | Int. G-3c | 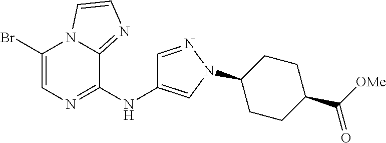 [SMILES: COC(=O)[C@H]1CC[C@@H](n2cc(Nc3ncc(Br)n4ccnc34)cn2)CC1] | 85% yield, brown solid [M + H]⁺ = 419.0; 421.0. ¹H NMR (400 MHz, DMSO-d₆) δ ppm 1.63-1.74 (m, 2 H), 1.85-1.94 (m, 4 H), 2.01-2.07 (m, 2 H), 2.64-2.75 (m, 1 H), 3.64 (s, 3 H), 4.13-4.25 (m, 1 H), 7.60 (s, 1 H), 7.68-7.71 (m, 1 H), 7.73-7.76 (m, 1 H), 7.96-8.00 (m, J = 1.0 Hz, 1 H), 8.14 (s, 1 H), 9.98 (s, 1 H). |
| Int. G- 2d | Int. D-1a | Int. G- 3d | 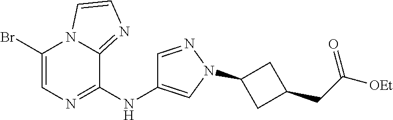 [SMILES: CCOC(=O)C[C@H]1C[C@@H](n2cc(Nc3ncc(Br)n4ccnc34)cn2)C1] | 48% yield, brown semi-solid [M + H]⁺ = 421.2. ¹H NMR (400 MHz, DMSO-d₆) δ ppm 1.20 (t, J = 7.2 Hz, 3 H), 2.12-2.21 (m, 2 H), 2.32-2.42 (m, 2 H), 2.53-2.61 (m, 3 H), 4.07 (q, J = 7.1 Hz, 2 H), 4.66-4.75 (m, 1 H), 7.59-7.61 (m, 1 H), 7.71 (d, J = 1.0 Hz, 1 H), 7.76-7.78 (m, 1 H), 7.99 (d, J = 1.2 Hz, 1 H), 8.17-8.19 (m, 1 H), 9.99-10.01 (m, 1 H). |
| Int. G-2e | Int. D-1a | Int. G-3e | 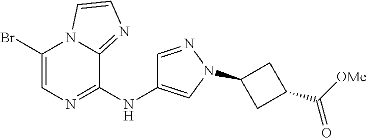 [SMILES: COC(=O)[C@H]1C[C@H](n2cc(Nc3ncc(Br)n4ccnc34)cn2)C1] | 54% yield, [M + H]⁺ = 391.0. ¹H NMR (400 MHz, DMSO-d₆) δ ppm 2.60-2.68 (m, 2 H), 2.71-2.81 (m, 2 H), 3.17-3.25 (m, 1 H), 3.69 (s, 3 H), 4.95- 5.06 (m, 1 H), 7.60 (s, 1 H), 7.71 (d, J = 1.0 Hz, 1 H), 7.83 (s, 1 H), 8.00 (d, J = 1.0 Hz, 1 H), 8.22 (s, 1 H), 10.02 (br s, 1 H). |

### Synthesis of TBM-G: Step 3

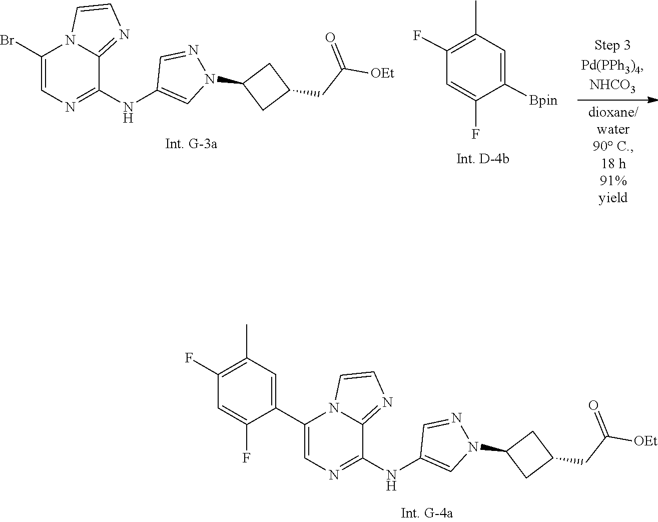

In a sealed tube, 2-(2,4-difluoro-5-methyl-phenyl)-4,4,5,5-tetramethyl-1,3,2-dioxaborolane Int. D-4b (485 mg, 1.9 mmol, 1.5 eq.), ethyl 2-((trans)-3-(4-((5-bromoimidazo[1,2-a]pyrazin-8-yl)amino)-1H-pyrazol-1-yl)cyclobutyl)acetate Int. G-3a (533 mg, 1.3 mmol, 1 eq.) and NaHCO₃ (320 mg, 3.8 mmol, 3 eq.) were added in 1,4-dioxane (3 mL, 0.32 M) and water (1 mL, 0.32 M). Nitrogen was bubbled into the reaction for 10 min. Then, Pd(PPh₃)₄ (220 mg, 0.19 mmol, 0.15 eq.) was added and nitrogen was bubbled for an additional 10 min. The resulting solution was stirred at 90° C. in a sealed tube under nitrogen atmosphere for 18 h. The reaction mixture was filtered over Celite, rinsed with EtOAc and concentrated. The resultant residue was purified by reverse phase column chromatography (5% to 100% MeOH in water w/ 0.1% formic acid). The fractions were combined and concentrated to afford ethyl 2-((trans)-3-(4-((5-(2,4-difluoro-5-methylphenyl)imidazo[1,2-a]pyrazin-8-yl)amino)-1H-pyrazol-1-yl)cyclobutyl)acetate 9nt. G-4a (562 mg, 9100 yield) as a yellow semi-solid.

LCMS: [M+H]⁺=467.2.

¹H NMR (400 MHz, DMSO-d₆) δ ppm 1.19 (t, J=7.1 Hz, 3H), 2.20 (br t, J=8.7 Hz, 2H), 2.29 (s, 3H), 2.55-2.69 (m, 5H), 4.07 (q, J=7.1 Hz, 2H), 4.95-5.05 (m, 1H), 7.42-7.50 (m, 2H), 7.58-7.64 (m, 3H), 7.84 (s, 1H), 8.23 (s, 1H), 10.00 (s, 1H).

TABLE 31
List of Int. G-4 compounds generated by Representative Procedure TBM-G:
Step 3

| Starting | | | | | |
|---|---|---|---|---|---|
| Material | | Product | | | |
| Int. | Int. | Int. | | | |
| G-3 | D | G-4 | Structure | Characterization | Notes |
| Int. G-3a | Int. D-4b | Int. G-4a | 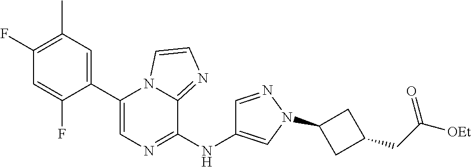 [SMILES: CCOC(=O)C[C@H]1C[C@H](n2cc(Nc3ncc(-c4cc(C)c(F)cc4F)n4ccnc34)cn2)C1] | 91% yield, yellow semi-solid [M + H]⁺ = 467.2. ¹H NMR (400 MHz, DMSO-d₆) δ ppm 1.19 (t, J = 7.1 Hz, 3 H), 2.20 (br t, J = 8.7 Hz, 2 H), 2.29 (s, 3 H), 2.55-2.69 (m, 5 H), 4.07 (q, J = 7.1 Hz, 2 H), 4.95-5.05 (m, 1 H), 7.42-7.50 (m, 2 H), 7.58-7.64 (m, 3 H), 7.84 (s, 1 H), 8.23 (s, 1 H), 10.00 (s, 1 H). | Conditions: Pd(PPh₃)₄, NaHCO₃, dioxane/ water, 90° C., 18 h |
| Int. G-3b | Int. D-4b | Int. G-4b | 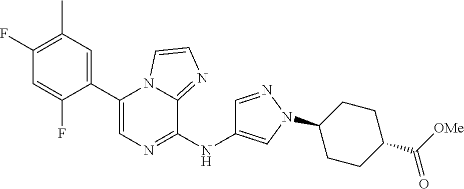 [SMILES: COC(=O)[C@H]1CC[C@H](n2cc(Nc3ncc(-c4cc(C)c(F)cc4F)n4ccnc34)cn2)CC1] | 90% yield, yellow semi-solid [M + H]⁺ = 467.2. ¹H NMR (400 MHz, DMSO-d₆) δ ppm 1.48-1.62 (m, 2 H), 1.73-1.86 (m, 2 H), 1.99-2.11 (m, 4 H), 2.29 (s, 3 H), 2.38- 2.47 (m, 1 H), 3.62 (s, 3 H), 4.12-4.23 (m, 1 H), 7.40-7.49 (m, 2 H), 7.58-7.66 (m, 3 H), 7.79 (s, 1 H), 8.21 (s, 1 H), 9.98 (s, 1 H). | Conditions: Pd(PPh₃)₄, NaHCO₃, dioxane/ water, 90° C., 18 h |
| Int. G-3c | Int. D-4b | Int. G-4c | 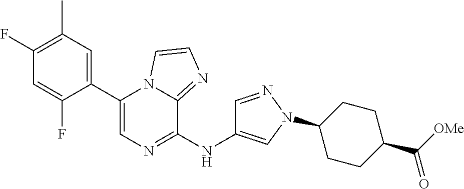 [SMILES: COC(=O)[C@H]1CC[C@@H](n2cc(Nc3ncc(-c4cc(C)c(F)cc4F)n4ccnc34)cn2)CC1] | 62% yield, yellow semi-solid [M + H]⁺ = 467.4 ¹H NMR (400 MHz, CHLOROFORM-d) δ ppm 1.25-1.29 (m, 1 H), 1.69-1.79 (m, 2 H), 2.06-2.12 (m, 3 H), 2.24-2.32 (m, 2 H), 2.32-2.36 (m, 3 H), 2.65-2.73 (m, 1 H), 3.71 (s, 3 H), 4.16-4.24 (m, 1 H), 6.98 (t, J = 9.4 Hz, 1 H), 7.29-7.37 (m, 2 H), 7.40-7.46 (m, 1 H), 7.56 (d, J = 1.2 Hz, 1 H), 7.62- 7.68 (m, 1 H), 7.80- 7.87 (m, 1 H), 8.19 (s, 1 H). ¹⁹F NMR (377 MHz, CHLOROFORM-d) δ ppm −111.51- −111.37 (m, 1 F), −110.30-−110.09 (m, 1 F). | Conditions: Pd(PPh₃)₄, NaHCO₃, dioxane/ water, 90° C., 18 h |
| Int. G-3d | Int. D-4b | Int. G-4d | 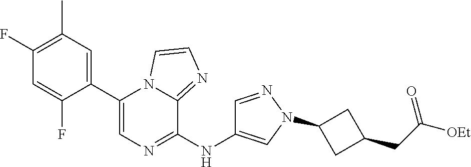 [SMILES: CCOC(=O)C[C@H]1C[C@@H](n2cc(Nc3ncc(-c4cc(C)c(F)cc4F)n4ccnc34)cn2)C1] | 70% yield, yellow oil [M + H]⁺ = 467.2. ¹H NMR (400 MHz, DMSO-d₆) δ ppm 1.20 (t, J = 7.1 Hz, 3 H), 2.13-2.22 (m, 2 H), 2.29 (s, 3 H), 2.33-2.43 (m, 2 H), 2.54-2.61 (m, 3 H), 4.07 (q, J = 7.3 Hz, 2 H), 4.68-4.77 (m, 1 H), 7.43-7.50 (m, 2 H). 7.59-7.65 (m. 3 H), 7.82 (s, 1 H), 8.24 (s, 1 H), 10.00 (s, 1 H). | Conditions: Pd(PPh₃)₄, NaHCO₃, dioxane/ water, 90° C., 18 h |
| Int. G-3e | Int. D-4b | Int. G-4e | 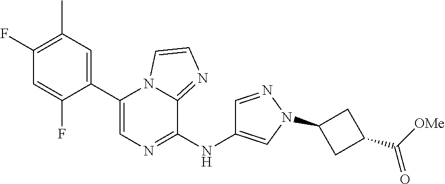 [SMILES: COC(=O)[C@H]1C[C@H](n2cc(Nc3ncc(-c4cc(C)c(F)cc4F)n4ccnc34)cn2)C1] | 89% yield, brown semi-solid [M + H]⁺ = 439.2. | Conditions: Pd(dppf)Cl₂, NaHCO₃, water/dioxane, 90° C., 2 h |

### Synthesis of TBM-G: Step 4

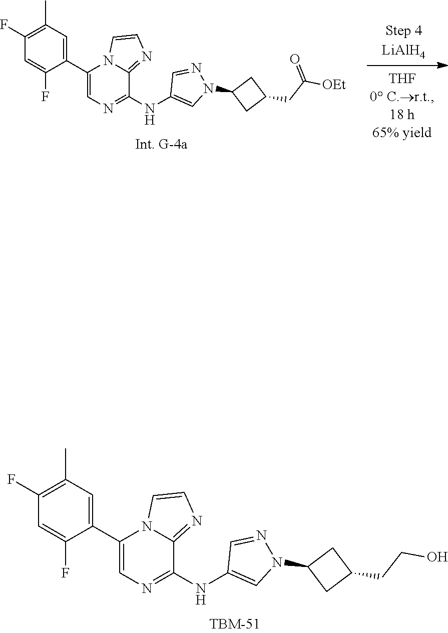 [SMILES: CCOC(=O)C[C@H]1C[C@H](n2cc(Nc3ncc(-c4cc(C)c(F)cc4F)n4ccnc34)cn2)C1 ; Cc1cc(-c2cnc(Nc3cnn([C@H]4C[C@H](CCO)C4)c3)c3nccn23)c(F)cc1F]

To a stirred solution of ethyl 2-((trans)-3-(4-((5-(2,4-difluoro-5-methylphenyl)imidazo[1,2-a]pyrazin-8-yl)amino)-1H-pyrazol-1-yl)cyclobutyl)acetate nt. G-4a (562 mg, 1.2 mmol, 1 eq.) in THE (8 mL) was added LiAlH₄ (137 mg, 3.61 mmol, 3 eq.) at 0 (C, overnight. The reaction was quenched at 0° C. with water (0.5 mL) and 1 N NaOH (0.5 mL) and stirred at room temperature for 20 min. The precipitate was filtered off and the filtrate was concentrated to afford 2-((trans)-3-(4-((5-(2,4-difluoro-5-methylphenyl)imidazo[1,2-a]pyrazin-8-yl)amino)-1H-pyrazol-1-yl)cyclobutyl)ethan-1-ol TBM-51 (451 mg, 65% yield) as a light-yellow oil, which was used without further purification.

LCMS: [M+H]⁺=425.2.

TABLE 32
List of TBM-G compounds generated by Representative Procedure TBM-G:
Step 4

| Starting | | | |
|---|---|---|---|
| Material | Product | | |
| Int. G-4 | TBM | Structure | Characterization |
| Int. G-4a | TBM-51 | 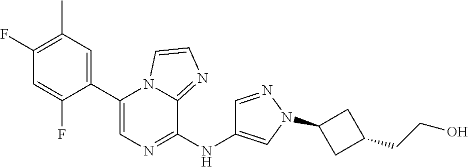 [SMILES: Cc1cc(-c2cnc(Nc3cnn([C@H]4C[C@H](CCO)C4)c3)c3nccn23)c(F)cc1F] | 65% yield, light yellow oil [M + H]⁺ = 425.2. |
| Int. G-4b | TBM-52 | 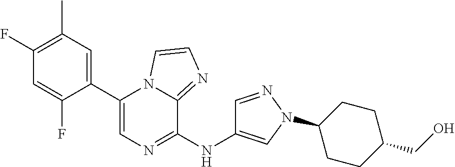 [SMILES: Cc1cc(-c2cnc(Nc3cnn([C@H]4CC[C@H](CO)CC4)c3)c3nccn23)c(F)cc1F] | 97% yield, brown oil [M + H]⁺ = 439.2. ¹H NMR (400 MHz, DMSO-d6) δ ppm 1.04-1.13 (m, 2 H), 1.39-1.47 (m, 1 H), 1.71 (qd, J = 12.6, 3.2 Hz, 2 H), 1.83-1.90 (m, 2 H), 2.02- 2.09 (m, 2 H), 2.29 (s, 3 H), 3.26 (t, J = 5.7 Hz, 2 H), 4.06-4.14 (m, 1 H), 4.45 (t, J = 5.3 Hz, 1 H), 7.41-7.49 (m, 2 H), 7.57-7.64 (m, 3 H), 7.78 (s, 1 H), 8.21 (s, 1 H), 9.96 (s, 1 H). ¹⁹F NMR (377 MHz, DMSO-d₆) δ ppm −112.16 (q, J = 9.5 Hz, 1 F), − 111.28 (q, J = 8.2 Hz, 1 F). |
| Int. G-4c | TBM-53 | 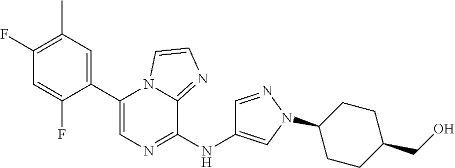 [SMILES: Cc1cc(-c2cnc(Nc3cnn([C@H]4CC[C@@H](CO)CC4)c3)c3nccn23)c(F)cc1F] | 74% yield, light-yellow oil [M + H]⁺ = 439.2. ¹H NMR (400 MHz, DMSO-d6) δ ppm 1.52-1.61 (m, 3 H), 1.64-1.89 (m, 3 H), 1.98-2.10 (m, 2 H), 2.29 (s, 3 H), 4.14-4.27 (m, 1 H), 4.35- 4.54 (m, 1 H), 7.41-7.48 (m, 2 H), 7.58-7.65 (m, 3 H), 7.77-7.82 (m, 1 H), 8.20-8.28 (m, 1 H), 9.97 (s, 1 H). Three protons are not apparent in the ¹H NMR spectrum. ¹⁹F NMR (377 MHz, DMSO-d₆) δ ppm −112.13 (q, J = 9.1 Hz, 1 F), − 111.28 (q, J = 8.2 Hz, 1 F). |
| Int. G-4d | TBM-54 | 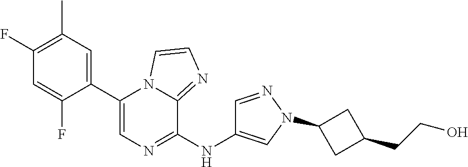 [SMILES: Cc1cc(-c2cnc(Nc3cnn([C@H]4C[C@@H](CCO)C4)c3)c3nccn23)c(F)cc1F] | 92% yield, orange semi-solid [M + H]⁺ = 425.2. ¹H NMR (400 MHz, DMSO-d6) δ ppm 1.18 (t, J = 7.2 Hz, 2 H), 1.62 (q, J = 6.4 Hz, 2 H), 2.29 (s, 3 H), 2.53-2.56 (m, 1 H), 3.36-3.44 (m, 2 H), 4.03 (q, J = 7.1 Hz, 1 H), 4.34- 4.41 (m, 1 H), 4.64-4.73 (m, 1 H), 7.41-7.50 (m, 2 H), 7.57-7.68 (m, 3 H), 7.82 (s, 1 H), 8.22 (s, 1 H), 9.98 (s, 1 H). |
| Int. G-4e | TBM-55 | 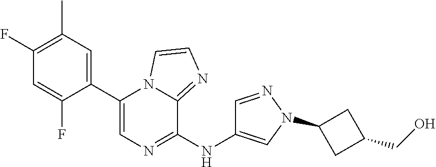 [SMILES: Cc1cc(-c2cnc(Nc3cnn([C@H]4C[C@H](CO)C4)c3)c3nccn23)c(F)cc1F] | 94% yield, brown solid [M + H]⁺ = 411.2. ¹H NMR (400 MHz, DMSO-d₆) δ ppm 2.21-2.28 (m, 2 H), 2.29 (s, 3 H), 2.33-2.44 (m, 1 H), 2.52-2.56 (m, 1 H), 3.51-3.57 (m, 2 H), 4.69 (br d, J = 3.7 Hz, 1 H), 4.86-4.95 (m, 1 H), 7.43-7.49 (m, 2 H), 7.61- 7.64 (m, 3 H), 7.84 (s, 1 H), 8.24 (s, 1 H), 9.99 (s, 1 H). |

### Example S17. General Procedure for TBM-H

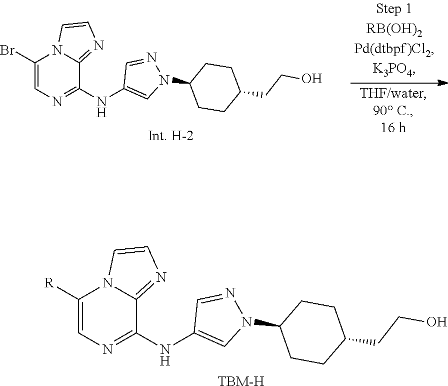

### Synthesis of TBM-H: Step 1

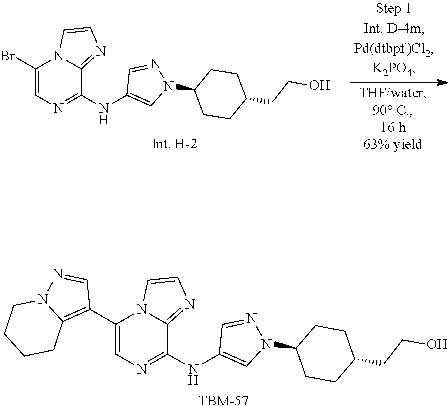

In a sealed tube, a mixture of benzyl 3-(4,4,5,5-tetramethyl-1,3,2-dioxaborolan-2-yl)-4,5,6,7-tetrahydropyrazolo[1,5-a]pyridine Int. D-4m (146.93 mg, 0.59 mmol), 2-((trans)-4-(4-((5-bromoimidazo[1,2-a]pyrazin-8-yl)amino)-1H-pyrazol-1-yl)cyclohexyl)ethan-1-ol Int. H-2 (200 mg, 0.49 mmol) and K₃PO₄ (313 mg, 1.48 mmol) in THE (3.7 mL) and water (1.3 mL) was sparged with nitrogen for 10 min; then Pd(dtbpf)Cl₂ (32 mg, 0.05 mmol) was added and the mixture was sparged again with nitrogen for 10 minutes. The mixture was then stirred at 90° C. for 16 h. The mixture was cooled down to room temperature and water was added. The aqueous phase was extracted thrice with EtOAc and the organic phases were combined, washed with water and brine, dried over Na₂SO₄, filtered, and concentrated to dryness. The resulting residue was purified by reverse phase column chromatography (5 to 100% ACN in water w/0.1% formic acid) to afford 2-((trans)-4-(4-((5-(4,5,6,7-tetrahydropyrazolo[1,5-a]pyridin-3-yl)imidazo[1,2-a]pyrazin-8-yl)amino)-1H-pyrazol-1-yl)cyclohexyl)ethan-1-ol TBM-57 (137.3 mg, 63% yield) as an orange oil.

LCMS: [M+H]⁺=447.2.

¹H NMR (400 MHz, DMSO-d₆) δ ppm 1.38 (q, J=6.4 Hz, 2H), 1.64-1.76 (m, 2H), 1.79-1.88 (m, 4H), 1.99-2.06 (m, 4H), 2.75 (t, J=6.2 Hz, 2H), 3.17 (d, J=3.7 Hz, 2H), 3.43-3.48 (m, 2H), 4.04-4.13 (m, 2H), 4.16 (t, J=6.0 Hz, 2H), 4.33-4.38 (m, 1H), 7.30 (s, 1H), 7.61 (d, J=1.2 Hz, 1H), 7.74 (s, 1H), 7.78 (d, J=1.2 Hz, 1H), 7.80 (s, 1H), 8.19-8.20 (m, 1H), 9.76 (s, 1H).

TABLE 33
List of intermediates used in TBM-H: Step 1
Starting Material

| Int. H | Structure | Source |
|---|---|---|
| Int. H-2 | 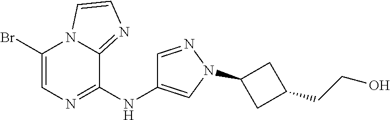 [SMILES: OCC[C@H]1C[C@H](n2cc(Nc3ncc(Br)n4ccnc34)cn2)C1] | Synthesis provided below |
| Int. H-3 | 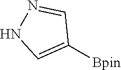 | Commercially available |

### Synthesis of 2-((trans)-4-(4-((5-bromoimidazo[1,2-a]pyrazin-8-yl)amino)-1H-pyrazol-1-yl)cyclohexyl)ethan-1-ol (Int. H-2)

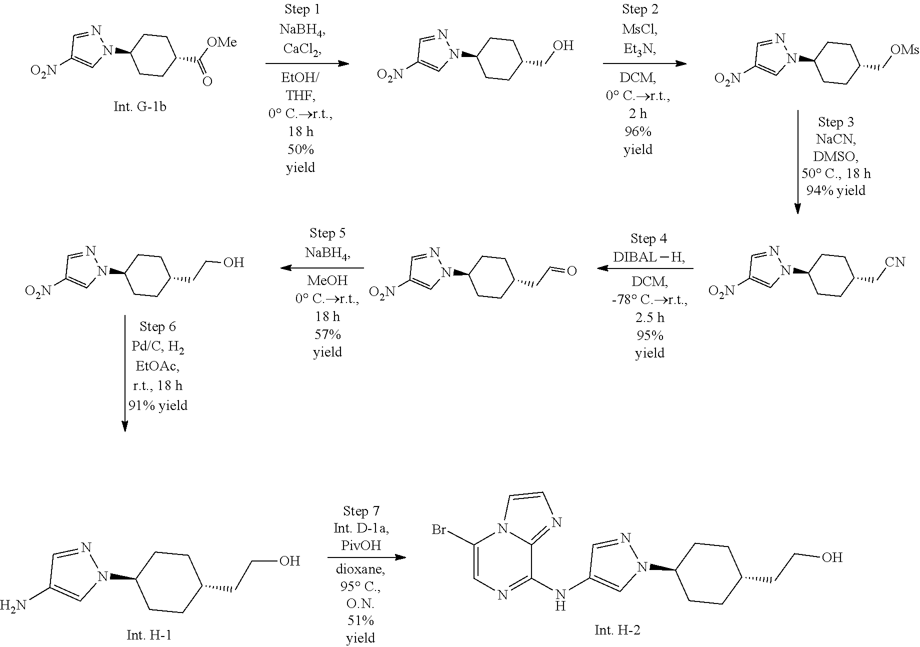

Step 1: A solution of methyl (trans)-4-(4-nitro-1H-pyrazol-1-yl)cyclohexane-1-carboxylate Int. G-1b (7.38 g, 29.1 mmol, 1.0 equiv) was dissolved in 2:1 MeOH/THF (150 mL) and cooled to 0° C., then calcium chloride (6.47 g, 58.3 mmol, 2.0 equiv) and sodium borohydride (4.41 g, 117 mmol, 4.0 equiv) were added sequentially. The reaction mixture was stirred at room temperature overnight. Water (50 mL) and brine (50 mL) were added to the mixture while stirring vigorously. The aqueous phase was extracted with EtOAc (3×100 mL) and the combined organic layers were dried over MgSO₄, filtered, and evaporated under reduced pressure. The residue was purified by normal phase column chromatography (0% to 100% EtOAc in heptane). The fractions were collected and concentrated, affording ((trans)-4-(4-nitro-1H-pyrazol-1-yl)cyclohexyl)methanol (3.3 g, 14.6 mmol, 50% yield) as a yellow solid.

LCMS: [M+H]⁺=276.2.

¹H NMR (400 MHz, DMSO-d₆) δ ppm 1.08 (qd, J=12.8, 3.2 Hz, 2H), 1.34-1.50 (m, 1H), 1.75 (qd, J=12.5, 3.5 Hz, 2H), 1.86 (br d, J=11.6 Hz, 2H), 2.00-2.12 (m, 2H), 3.25 (t, J=5.7 Hz, 2H), 4.20 (tt, J=11.9, 3.9 Hz, 1H), 4.47 (t, J=5.3 Hz, 1H), 8.25 (s, 1H), 8.90 (s, 1H).

Step 2: Methanesulfonyl chloride (1.25 mL, 16.1 mmol, 1.1 equiv) was added dropwise to a cooled (0° C.) solution of ((trans)-4-(4-nitro-1H-pyrazol-1-yl)cyclohexyl)methanol (3.3 g, 14.6 mmol, 1.0 equiv) and triethylamine (2.66 mL, 19.0 mmol, 1.3 equiv) in DCM (73 mL). The reaction mixture was stirred at room temperature for 2 hours. Water (100 mL) was added to the reaction mixture, the phases were separated, and the aqueous layer was extracted with DCM (3×50 mL). The organic layers were combined, washed with 1.0 M HCl_{(aq)} (3×50 mL) and brine (50 mL), then dried over MgSO₄, filtered, and evaporated under reduced pressure to afford ((trans)-4-(4-nitro-1H-pyrazol-1-yl)cyclohexyl)methyl methanesulfonate (4.25 g, 14.0 mmol, 96% yield) as a yellow solid, which was used without further purification.

LCMS: [M+H]⁺=304.2.

¹H NMR (400 MHz, DMSO-d₆) δ ppm 1.16-1.29 (m, 2H), 1.66-1.83 (m, 3H), 1.88 (br d, J=12.0 Hz, 2H), 2.09 (br dd, J=12.8, 3.5 Hz, 2H), 3.18 (s, 3H), 4.08 (d, J=6.2 Hz, 2H), 4.24 (tt, J=11.8, 3.9 Hz, 1H), 8.26 (s, 1H), 8.92 (s, 1H).

Step 3: A solution of ((trans)-4-(4-nitro-1H-pyrazol-1-yl)cyclohexyl)methyl methanesulfonate (4.25 g, 14.0 mmol, 1.0 equiv) and sodium cyanide (1.58 g, 32.2 mmol, 2.3 equiv) in DMSO (45 mL) was stirred at 50° C. overnight. The reaction mixture was cooled down to room temperature and then water (100 mL) and EtOAc (50 mL) were added. The phases were separated and the aqueous layer was extracted with EtOAc (3×50 mL). The organic layers were combined, washed with saturated NaHCO_{3(aq)} (50 mL) and brine (50 mL), dried over MgSO₄, filtered, and evaporated under reduced pressure to afford 2-((trans)-4-(4-nitro-1H-pyrazol-1-yl)cyclohexyl)acetonitrile (3.1 g, 13.2 mmol, 94% yield) as an orange oil, which was used without further purification.

LCMS: [M+H]⁺=285.2.

¹H NMR (400 MHz, DMSO-d₆) δ ppm 1.26 (qd, J=12.7, 3.3 Hz, 2H), 1.63-1.75 (m, 1H), 1.75-1.86 (m, 2H), 1.89 (br d, J=12.0 Hz, 2H), 2.03-2.13 (m, 2H), 2.51-2.55 (m, 2H), 4.24 (tt, J=11.8, 3.9 Hz, 1H), 8.26 (s, 1H), 8.90 (s, 1H).

Step 4: DIBAL-H 1.0 M in DCM (39.7 mL, 39.7 mmol, 3.0 equiv) was added dropwise to a solution of 2-((trans)-4-(4-nitro-1H-pyrazol-1-yl)cyclohexyl)acetonitrile (3.1 g, 13.2 mmol, 1.0 equiv) in DCM (66 mL) cooled to −78° C. (dry ice/acetone bath) under nitrogen atmosphere. The reaction mixture was stirred at −78° C. for 2.5 hours. Aqueous Rochelle's salt (50 mL) was carefully added to the reaction mixture, which was stirred at room temperature overnight. EtOAc (150 mL) was added, and then the organic phase was washed with 1.0 M HCl_{(aq)} (3×20 mL), dried over MgSO₄, filtered, and evaporated under reduced pressure to yield 2-((trans)-4-(4-nitro-1H-pyrazol-1-yl)cyclohexyl)acetaldehyde (3.42 g, 12.6 mmol, 95% yield) as an orange oil, which was used without further purification.

LCMS: [M+H]⁺=238.2.

¹H NMR (400 MHz, DMSO-d₆) δ ppm 1.12-1.20 (m, 2H), 1.74-1.84 (m, 5H), 2.02-2.06 (m, 2H), 2.37 (dd, J=6.6, 1.8 Hz, 2H), 4.19-4.26 (m, 1H), 8.25 (s, 1H), 8.91 (s, 1H), 9.68 (t, J=1.9 Hz, 1H).

Step 5: Sodium borohydride (998 mg, 26.4 mmol, 2.0 equiv) was added in one portion to a cooled suspension (0° C.) of 2-((trans)-4-(4-nitro-1H-pyrazol-1-yl)cyclohexyl)acetaldehyde (3.13 g, 13.2 mmol, 1.0 equiv) in methanol (66 mL). The reaction mixture was stirred overnight at room temperature. Water (80 mL) was added to the reaction mixture, and then the aqueous layer was extracted with EtOAc (3×50 mL). The organic layers were combined, washed with brine (50 mL), dried over MgSO₄, filtered, and evaporated under reduced pressure. The crude residue was purified by silica gel column chromatography (0% to 100% EtOAc in heptane) to afford 2-((trans)-4-(4-nitro-1H-pyrazol-1-yl)cyclohexyl)ethan-1-ol (1.81 g, 7.56 mmol, 57% yield) as a light yellow solid.

LCMS: [M+H]⁺=240.2.

¹H NMR (400 MHz, DMSO-d₆) δ ppm 1.02-1.14 (m, 2H), 1.36 (q, J=6.6 Hz, 2H), 1.39-1.53 (m, 1H), 1.67-1.80 (m, 2H), 1.84 (br d, J=12.0 Hz, 2H), 1.98-2.11 (m, 2H), 3.38-3.53 (m, 2H), 4.21 (tt, J=11.9, 3.9 Hz, 1H), 4.35 (t, J=5.1 Hz, 1H), 8.25 (s, 1H), 8.90 (s, 1H).

Step 6: 2-((trans)-4-(4-Nitro-1H-pyrazol-1-yl)cyclohexyl)ethan-1-ol (1.81 g, 7.56 mmol, 1.0 equiv) was dissolved in ethyl acetate (38 mL), the solution was then degassed by bubbling nitrogen under sonication for 20 minutes. Pd/C 10% w/w (1.61 g, 1.51 mmol, 0.2 equiv) was added, then the mixture was further degassed by bubbling nitrogen under sonication for 20 minutes. The nitrogen balloon was replaced with one filled with hydrogen, which was bubbled through the reaction mixture for 10 minutes, then the reaction mixture was stirred under a static hydrogen atmosphere overnight. The reaction mixture was filtered through celite and the celite was washed thoroughly with EtOAc and MeOH. The solvent was evaporated under reduced pressure to afford 2-((trans)-4-(4-amino-1H-pyrazol-1-yl)cyclohexyl)ethan-1-ol Int. H-1 (1.45 g, 6.92 mmol, 91% yield) as a purple solid, which was used without further purification.

LCMS: [M+H]⁺=210.2.

¹H NMR (400 MHz, DMSO-d₆) δ ppm 0.98-1.12 (m, 2H), 1.31-1.37 (m, 2H), 1.38-1.47 (m, 1H), 1.60 (br dd, J=12.3, 3.3 Hz, 2H), 1.79 (br d, J=12.7 Hz, 2H), 1.88-1.98 (m, 2H), 3.38-3.52 (m, 2H), 3.58-4.19 (br s, 2H), 3.87 (tt, J=11.9, 3.8 Hz, 1H), 4.33 (t, J=5.1 Hz, 1H), 6.87 (s, 1H), 7.02 (s, 1H).

Step 7: 5,8-Dibromoimidazo[1,2-a]pyrazine Int. D-1a (2.41 g, 8.69 mmol) and pivalic acid (2.13 g, 20.85 mmol) were successively added to a solution of 2-((trans)-4-(4-amino-1H-pyrazol-1-yl)cyclohexyl)ethan-1-ol Int. H-1 (2.0 g, 9.56 mmol) in 1,4-dioxane (43.44 mL). The mixture was heated to 95° C. in an oil bath overnight. After cooling to room temperature, the reaction was partitioned between EtOAc and H₂N and the aqueous layer was adjusted to pH 9 with 2 M NaOH. The aqueous layer was extracted with EtOAc (3×), and the combined organics were washed with brine, dried over MgSO₄, filtered, and concentrated. The crude residue was purified by reverse phase column chromatography (500 to 100% MeCN in water w/ 0.17 formic acid). The fractions were collected and concentrated, affording 2-((trans)-4-(4-((5-bromoimidazo[1,2-a]pyrazin-8-yl)amino)-1H-pyrazol-1-yl)cyclohexyl)ethan-1-ol Int. H-2 (1.28 g, 5100 yield) as a brown solid.

LCMS: [M+H]⁺=405.0.

¹H NMR (400 MHz, DMSO-d₆) δ ppm 1.11 (br dd, J=14.3, 11.4 Hz, 2H), 1.37 (q, J=6.8 Hz, 2H), 1.41-1.51 (m, 1H), 1.70 (qd, J=12.5, 3.3 Hz, 2H), 1.83 (br d, J=13.7 Hz, 2H), 2.02 (br d, J=12.2 Hz, 2H), 3.39-3.52 (m, 2H), 4.08 (tt, J=11.9, 3.9 Hz, 1H), 4.34 (t, J=5.1 Hz, 1H), 7.59 (s, 1H), 7.70 (d, J=1.0 Hz, 1H), 7.73 (s, 1H), 7.98 (d, J=1.0 Hz, 1H), 8.14 (s, 1H), 9.97 (s, 1H).

TABLE 34
List of TBM-H compounds generated by Representative Procedure TBM-H:
Step 1

| Starting | | | | |
|---|---|---|---|---|
| Material | Product | | | |
| RB(OH)₂ | TBM | Structure | Characterization | Note |
| 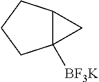 Int. D-4o | TBM- 56 | 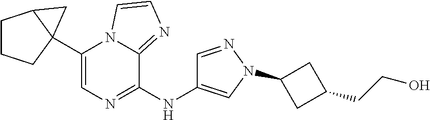 [SMILES: OCC[C@H]1C[C@H](n2cc(Nc3ncc(C45CCCC4C5)n4ccnc34)cn2)C1] | 76% yield, off-white solid [M + H]⁺ = 407.2 ¹H NMR (400 MHz, DMSO-d6) δ ppm 0.76- 0.81 (m, 1 H), 0.99 (t, J = 4.4 Hz, 1 H), 1.05- 1.16 (m, 2 H), 1.34- 1.45 (m, 4 H), 1.64- 1.75 (m, 4 H), 1.81- 1.88 (m, 4 H), 2.01- 2.10 (m, 3 H), 3.46 (br t, J = 6.2 Hz, 2 H), 4.02- 4.09 (m, 1 H), 4.35 (br s, 1 H), 7.31 (s, 1 H), 7.64 (d, J = 0.7 Hz, 1 H), 7.72 (s, 1 H), 7.85 (d, J = 0.7 Hz, 1 H), 8.14 (s, 1 H), 9.64 (s, 1 H). | Conditions: Catac XiumA- Pd-G₃ Cs₂CO₃, toluene/ water 100° C., 18 h |
| 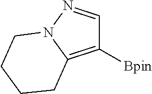 Int. D-4m | TBM- 57 | 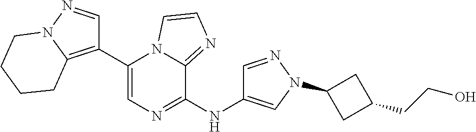 [SMILES: OCC[C@H]1C[C@H](n2cc(Nc3ncc(-c4cnn5c4CCCC5)n4ccnc34)cn2)C1] | 63% yield, orange oil [M + H]⁺ = 447.2 ¹H NMR (400 MHz, DMSO-d6) δ ppm 1.38 (q, J = 6.4 Hz, 2 H), 1.64-1.76 (m, 2 H), 1.79-1.88 (m, 4 H), 1.99-2.06 (m, 4 H), 2.75 (t, J = 6.2 Hz, 2 H), 3.17 (d, J = 3.7 Hz, 2 H), 3.43- 3.48 (m, 2 H), 4.04-4.13 (m, 2 H), 4.16 (t, J = 6.0 Hz, 2 H), 4.33-4.38 (m, 1 H), 7.30 (s, 1 H), 7.61 (d, J = 1.2 Hz, 1 H), 7.74 (s, 1 H), 7.78 (d, J = 1.2 Hz, 1 H), 7.80 (s, 1 H), 8.19-8.20 (m, 1 H), 9.76 (s, 1 H). | |
| 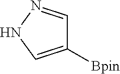 Int. H-3 | TBM- 58 | 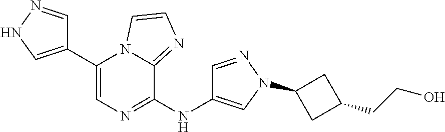 [SMILES: OCC[C@H]1C[C@H](n2cc(Nc3ncc(-c4cn[nH]c4)n4ccnc34)cn2)C1] | 40% yield, off-white solid [M + 1]⁺ = 393.4 ¹H NMR (400 MHz, DMSO-d6) δ ppm 1.03-1.18 (m, 2 H), 1.37 (q, J = 6.5 Hz, 2 H), 1.42- 1.53 (m, 1 H), 1.63- 1.77 (m, 2 H), 1.84 (br d, J = 13.0 Hz, 2 H), 2.03 (br d, J = 11.5 Hz, 2 H), 3.46 (br s, 2 H), 4.08 (tt, J = 11.8, 3.8 Hz, 1 H), 4.31-4.41 (m, 1 H), 7.56 (s, 1 H), 7.67 (s, 1 H), 7.75 (s, 1 H), 7.96-8.13 (m, 1 H), 8.05 (s, 1 H), 8.19 (s, 1 H), 8.24-8.49 (m, 1 H), 9.75 (s, 1 H), 13.28 (br s, 1 H). | Pd(dppf)Cl₂, NaHCO₃ DMF/water microwave 120° C., 30 min |

### Example S18. Procedure for TBM-59

### Synthesis of 2-((trans)-4-(4-((5-(2,4-difluoro-5-methylphenyl)imidazo[1,2-a]pyridin-8-yl)amino)-1H-pyrazol-1-yl)cyclohexyl)ethan-1-ol (TBM-59)

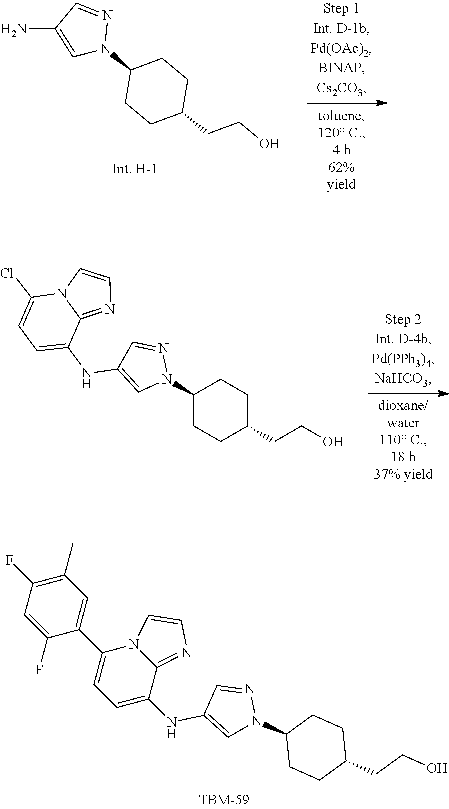

Step 1: In a sealed tube, 2-((trans)-4-(4-amino-1H-pyrazol-1-yl)cyclohexyl)ethan-1-ol Int. H-1 (250 mg, 1.19 mmol, 1.0 equiv), 5-chloro-8-iodoimidazo[1,2-a]pyridine Int. D-1b (332.6 mg, 1.19 mmol, 1.0 equiv), rac-BINAP (74.6 mg, 0.120 mmol, 0.1 equiv), and cesium carbonate (1.95 g, 5.97 mmol, 5.0 equiv) were mixed with toluene (9.5 mL), then nitrogen was bubbled through the mixture for 30 minutes under sonication. Pd(OAc)₂ (26.9 mg, 0.120 mmol, 0.1 equiv) was then quickly added and nitrogen bubbling was continued for additional 10 minutes. The tube was sealed and the reaction mixture was stirred at 120° C. for 4 hours. The reaction mixture was cooled to room temperature and the volatiles were evaporated. The crude material was purified by reverse phase column chromatography (5% to 100% MeOH in water w/0.1% formic acid). The fractions were collected and concentrated, affording 2-((trans)-4-(4-((5-chloroimidazo[1,2-a]pyridin-8-yl)amino)-1H-pyrazol-1-yl)cyclohexyl)ethan-1-ol (275 mg, 0.739 mmol, 62% yield) as a yellow semi-solid.

LCMS: [M+H]⁺=360.2.

¹H NMR (400 MHz, CDCl₃) δ ppm 1.10-1.24 (m, 2H), 1.56 (br t, J=4.6 Hz, 3H), 1.78 (qd, J=12.6, 3.1 Hz, 2H), 1.97 (br d, J=12.5 Hz, 2H), 2.01 (s, 1H), 2.24 (br d, J=11.6 Hz, 2H), 3.74 (br t, J=6.3 Hz, 1H), 3.77-3.98 (m, 2H), 4.10 (tt, J=12.0, 3.7 Hz, 1H), 6.53 (d, J=8.2 Hz, 1H), 6.79 (d, J=8.1 Hz, 1H), 7.49 (s, 1H), 7.52-7.67 (m, 3H), 7.75 (d, J=1.1 Hz, 1H), 8.19 (br s, 1H).

Step 2: In a sealed tube, 2-((trans)-4-(4-((5-chloroimidazo[1,2-a]pyridin-8-yl)amino)-1H-pyrazol-1-yl)cyclohexyl)ethan-1-ol (275. mg, 0.764 mmol, 1.0 equiv), 2-(2,4-difluoro-5-methyl-phenyl)-4,4,5,5-tetramethyl-1,3,2-dioxaborolane (Int. D-4b) (233 mg, 0.917 mmol, 1.2 equiv), and sodium bicarbonate (232 mg, 2.29 mmol, 3.0 equiv) were mixed with 1,4-dioxane (0.6 mL) and water (0.2 mL), then nitrogen was bubbled through the mixture for 30 minutes under sonication. Pd(PPh₃)₄ (177 mg, 0.153 mmol, 0.2 equiv) was then quickly added and nitrogen bubbling was continued for an additional 10 minutes. The tube was sealed and the reaction mixture was stirred at 110° C. for 18 h. The reaction mixture was cooled to room temperature, and then the volatiles were evaporated. The crude residue was purified by reverse phase column chromatography (5% to 100% MeOH in water w/ 0.1% formic acid). The fractions were collected and concentrated, affording 2-((trans)-4-(4-((5-(2,4-difluoro-5-methylphenyl)imidazo[1,2-a]pyridin-8-yl)amino)-1H-pyrazol-1-yl)cyclohexyl)ethan-1-ol TBM-59 (135 mg, 0.282 mmol, 37% yield) as a brown semi-solid.

LCMS: [M+H]⁺=452.2.

¹H NMR (400 MHz, DMSO-d₆) δ ppm 1.03-1.17 (m, 2H), 1.38 (q, J=6.6 Hz, 2H), 1.41-1.55 (m, 1H), 1.75 (qd, J=12.5, 3.2 Hz, 2H), 1.85 (br d, J=11.9 Hz, 2H), 2.05 (br d, J=10.6 Hz, 2H), 2.28 (s, 3H), 3.40-3.54 (m, 2H), 4.08 (tt, J=11.9, 3.9 Hz, 1H), 4.35 (t, J=5.0 Hz, 1H), 6.51 (d, J=7.8 Hz, 1H), 6.74 (d, J=7.7 Hz, 1H), 7.41 (t, J=10.0 Hz, 1H), 7.45 (d, J=1.8 Hz, 1H), 7.49-7.58 (m, 3H), 7.87 (s, 1H), 8.10 (s, 1H).

¹⁹F NMR (377 MHz, DMSO-d₆) δ ppm −112.35 (q, J=9.1 Hz, 1 F), −111.89 (q, J=8.2 Hz, 1 F).

### Example S19. Procedure for TBM-60

### Synthesis of 5-(2,4-difluoro-5-methylphenyl)-N-(1-(1-(2,2-dimethoxyethyl)piperidin-4-yl)-1H-pyrazol-4-yl)-N-methylimidazo[1,2-a]pyrazin-8-amine (TBM-60)

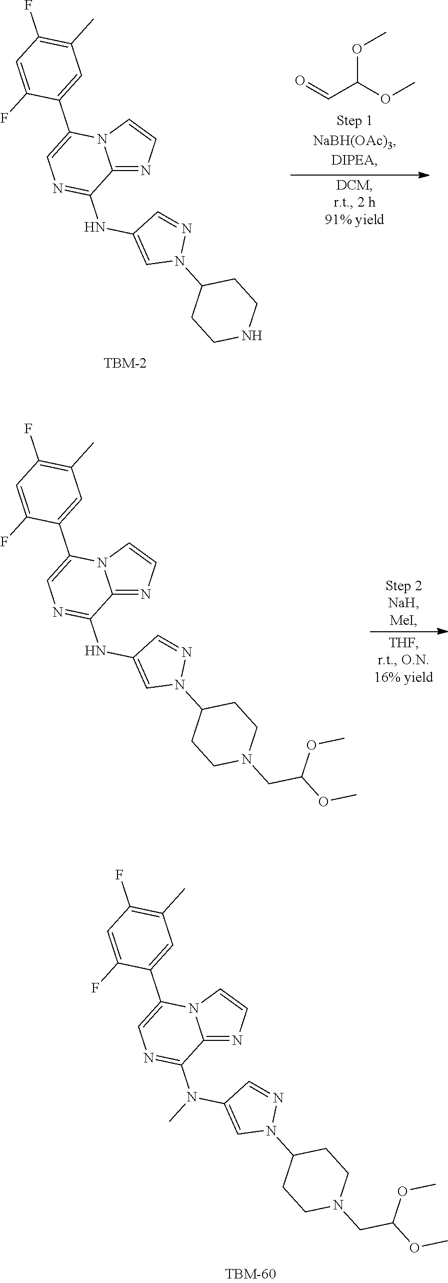

Step 1: To a solution of 5-(2,4-difluoro-5-methylphenyl)-N-(1-(piperidin-4-yl)-1H-pyrazol-4-yl)imidazo[1,2-a]pyrazin-8-amine TBM-2 (bis-HCl salt, 1.0 g, 2.07 mmol, 1 equiv.) in DCM (4.5 mL, 0.42 M) and DMSO (0.40 mL, 0.42 M) were added 2,2-dimethoxyacetaldehyde 60% w/w in H₂O (0.63 mL, 4.14 mmol, 2 equiv.) and NaBH(OAc)₃ (878.76 mg, 4.15 mmol, 2 equiv.). The reaction mixture was stirred at room temperature for 2 h. The solvent was evaporated and the residue was then purified by reverse phase column chromatography (5% to 40% MeOH in water w/0.1% formic acid). The fractions were collected and concentrated affording 5-(2,4-difluoro-5-methylphenyl)-N-(1-(1-(2,2-dimethoxyethyl)piperidin-4-yl)-1H-pyrazol-4-yl)imidazo[1,2-a]pyrazin-8-amine (936 mg, 91% yield) as a tan solid.

LCMS: [M+H]⁺=498.2.

¹H NMR (400 MHz, DMSO-d₆) δ ppm 1.87-2.02 (m, 4H), 2.18-2.27 (m, 2H), 2.29 (s, 3H), 3.00 (br d, J=11.7 Hz, 2H), 3.27 (s, 6H), 4.08-4.17 (m, 1H), 4.50 (t, J=5.0 Hz, 1H), 7.42-7.48 (m, 2H), 7.58-7.65 (m, 3H), 7.80 (s, 1H), 8.14 (s, 1H), 8.23 (s, 1H), 9.98 (s, 1H).

¹⁹F NMR (377 MHz, DMSO-d₆) δ ppm −112.17 (q, J=9.5 Hz, 1 F), −111.25 (q, J=8.2 Hz, 1 F).

Step 2: To a solution of 5-(2,4-difluoro-5-methylphenyl)-N-(1-(1-(2,2-dimethoxyethyl)piperidin-4-yl)-1H-pyrazol-4-yl)imidazo[1,2-a]pyrazin-8-amine (250 mg, 0.5000 mmol, 1.0 equiv.) in THE (5 mL) was added NaH (24.12 mg, 0.6000 mmol, 1.2 equiv.) at 0° C. The reaction was stirred at room temperature for 15-20 min. Then, Mel (0.03 mL, 0.5300 mmol, 1.05 equiv.) was added. The reaction mixture was stirred at room temperature overnight. LCMS showed starting material and a mixture of 3 products, two of which had the expected mass. Mel (0.03 mL, 0.5300 mmol, 1.05 equiv.) was added and after 2 h the reaction was quenched with water and extracted with EtOAc (3×). The combined organic layers were dried over sodium sulfate, filtered, and concentrated. The crude product was purified by reverse phase column chromatography (5 to 100% MeCN in water w/ 0.1% formic acid) to give the product (80% purity by LCMS high pH). The mixture was further purified by reverse phase column chromatography (0% to 100% MeCN in water w/pH 10 buffer) to afford 5-(2,4-difluoro-5-methylphenyl)-N-(1-(1-(2,2-dimethoxyethyl)piperidin-4-yl)-1H-pyrazol-4-yl)-N-methylimidazo[1,2-a]pyrazin-8-amine TBM-60 (40 mg, 16%) as a white solid.

LCMS: [M+H]⁺=512.3.

¹H NMR (400 MHz, DMSO-d₆) δ ppm 1.97 (br s, 4H), 2.16-2.24 (m, 2H), 2.28 (br s, 3H), 2.43-2.47 (m, 2H), 2.94-3.04 (m, 2H), 3.27 (br s, 6H), 3.97 (br s, 3H), 4.03-4.15 (m, 1H), 4.44-4.54 (m, 1H), 7.38-7.50 (m, 2H), 7.55-7.68 (m, 4H), 8.04-8.12 (m, 1H).

¹⁹F NMR (377 MHz, DMSO-d₆) δ ppm −112.15-−111.82 (m, 1 F), −111.14-−110.99 (m, 1 F).

### Procedures for Compound Nos. 1-120

### Example S20. Representative Procedure LDD-I

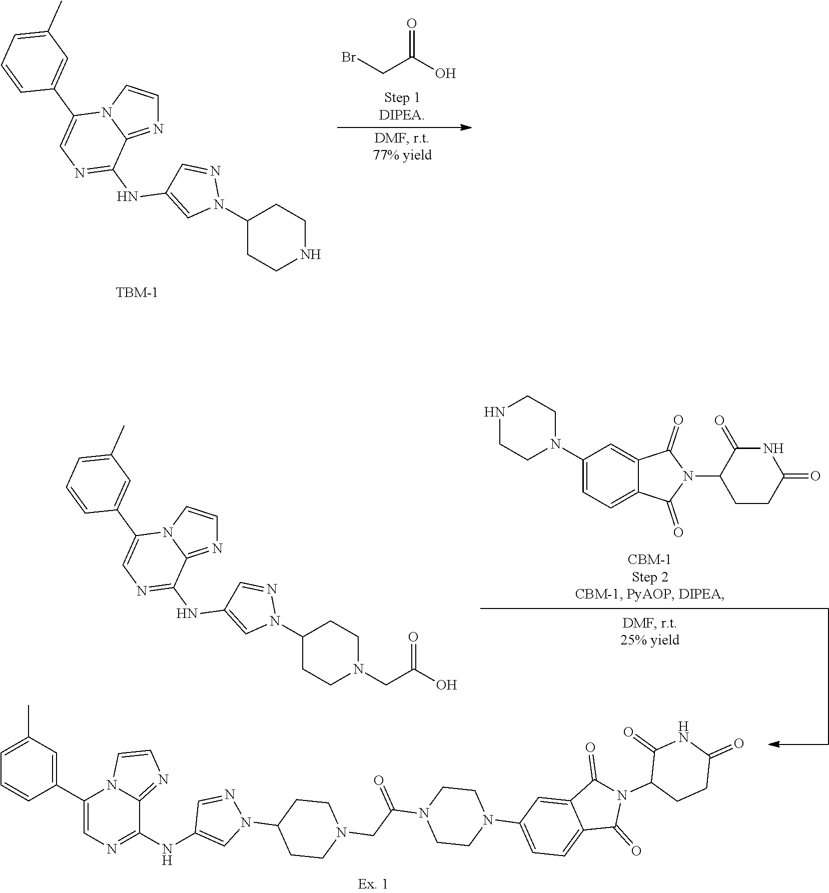 [SMILES: Cc1cccc(-c2cnc(Nc3cnn(C4CCNCC4)c3)c3nccn23)c1 ; O=C(O)CBr ; Cc1cccc(-c2cnc(Nc3cnn(C4CCN(CC(=O)O)CC4)c3)c3nccn23)c1 ; O=C1CCC(N2C(=O)c3ccc(N4CCNCC4)cc3C2=O)C(=O)N1 ; Cc1cccc(-c2cnc(Nc3cnn(C4CCN(CC(=O)N5CCN(c6ccc7c(c6)C(=O)N(C6CCC(=O)NC6=O)C7=O)CC5)CC4)c3)c3nccn23)c1]

Step 1: TBM-1 (68.0 mg, 0.16 mmol) was dissolved in a mixture of DMF (0.80 mL) and DIPEA (113 μL, 0.65 mmol), then 2-bromoacetic acid (14.0 μL, 0.19 mmol) was added and the solution was stirred at room temperature. HPLC showed full conversion into the product and a small impurity. The mixture was purified by reverse phase column chromatography (5% to 100% MeOH in water w/0.1% formic acid). The fractions were combined and evaporated to afford 2-(4-(4-((5-(m-tolyl)imidazo[1,2-a]pyrazin-8-yl)amino)-1H-pyrazol-1-yl)piperidin-1-yl)acetic acid (54 mg, 77% yield), as a white solid.

LCMS: [M+H]⁺=433.2.

¹H NMR (400 MHz, DMSO-d₆) δ ppm 1.99-2.08 (m, 4H), 2.41 (s, 3H), 2.53-2.59 (m, 2H), 3.10 (br d, J=11.5 Hz, 3H), 4.16-4.24 (m, 1H), 7.31-7.34 (m, 1H), 7.43 (s, 1H), 7.45-7.52 (m, 3H), 7.64 (d, J=1.0 Hz, 1H), 7.81 (s, 1H), 7.89 (d, J=1.0 Hz, 1H), 8.25 (s, 1H), 9.90 (s, 1H).

Step 2: To a solution of CBM-1 (53.4 mg, 0.12 mmol), DIPEA (102 μL, 0.59 mmol) and 2-(4-(4-((5-(m-tolyl)imidazo[1,2-a]pyrazin-8-yl)amino)-1H-pyrazol-1-yl)piperidin-1-yl)acetic acid (53 mg, 0.12 mmol) in DMF (1 mL) was added PyAOP (61 mg, 0.12 mmol) at 0° C. The mixture was stirred at r.t. for 3 hours. The mixture was purified by reverse phase column chromatography (5% to 100% MeCN in water w/0.1% formic acid). The fractions were collected and evaporated to afford 2-(2,6-dioxopiperidin-3-yl)-5-(4-(2-(4-(4-((5-(m-tolyl)imidazo[1,2-a]pyrazin-8-yl)amino)-1H-pyrazol-1-yl)piperidin-1-yl)acetyl)piperazin-1-yl)isoindoline-1,3-dione (22.4 mg, 25% yield, formic acid salt) as a light-yellow solid.

LCMS: [M+H]⁺=756.2.

¹H NMR (400 MHz, DMSO-d₆) δ ppm 1.90-2.06 (m, 5H), 2.23 (br. t, J=10.4 Hz, 2H), 2.40 (s, 3H), 2.53-2.62 (m, 2H), 2.83-2.90 (m, 1H), 2.92-2.99 (m, 2H), 3.25 (s, 2H), 3.46-3.51 (m, 2H), 3.53-3.59 (m, 2H), 3.59-3.65 (m, 2H), 3.74-3.80 (m, 2H), 4.09-4.19 (m, 1H), 5.07 (dd, J=12.8, 5.5 Hz, 1H), 7.25-7.34 (m, 2H), 7.38 (br. d, J=1.0 Hz, 1H), 7.42 (s, 1H), 7.44-7.47 (m, 2H), 7.50 (br. s, 1H), 7.64 (s, 1H), 7.70 (d, J=8.3 Hz, 1H), 7.80 (s, 1H), 7.89 s, 1H), 8.23 s, 1H), 9.88 s, 1H), 11.08 s, 1H).

TABLE 35
List of Examples prepared by procedure LDD-I

| Components | | Example | | | |
|---|---|---|---|---|---|
| TBM | CBM | Cmp | Structure | IUPAC Name | Characterization |
| TBM-1 | CBM-1 | 1 | 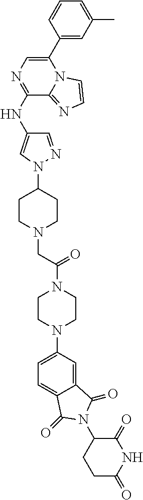 [SMILES: Cc1cccc(-c2cnc(Nc3cnn(C4CCN(CC(=O)N5CCN(c6ccc7c(c6)C(=O)N(C6CCC(=O)NC6=O)C7=O)CC5)CC4)c3)c3nccn23)c1] | 2-(2,6- dioxopiperidin- 3-yl)-5-(4-(2- (4-(4-((5-(m- tolyl)imidazo [1,2-a]pyrazin-8- yl)amino)-1H- pyrazol-1- yl)piperidin-1- yl)acetyl) piperazin-1- yl)isoindoline- 1,3-dione | 22.4 mg, 19% yield (2 steps), light- yellow solid [M + H]⁺ = 756.2 ¹H NMR (400 MHz, DMSO-d₆) δ ppm 1.90-2.06 (m, 5 H), 2.23 (br. t, J = 10.4 Hz, 2 H), 2.40 (s, 3 H), 2.53-2.62 (m, 2 H), 2.83-2.90 (m, 1 H), 2.92-2.99 (m, 2 H), 3.25 (s, 2 H), 3.46-3.51 (m, 2 H), 3.53- 3.59 (m, 2 H), 3.59-3.65 (m, 2 H), 3.74-3.80 (m, 2 H), 4.09-4.19 (m, 1 H), 5.07 (dd, J = 12.8, 5.5 Hz, 1 H), 7.25-7.34 (m, 2 H), 7.38 (br. d, J = 1.0 Hz, 1 H), 7.42 (s, 1 H), 7.44- 7.47 (m, 2 H), 7.50 (br. s, 1 H), 7.64 (s, 1 H), 7.70 (d, J = 8.3 Hz, 1 H), 7.80 (s, 1 H), 7.89 (s, 1 H), 8.23 (s, 1 H), 9.88 (s, 1 H), 11.08 (s, 1 H). |
| TBM-2 | CBM-1 | 6 | 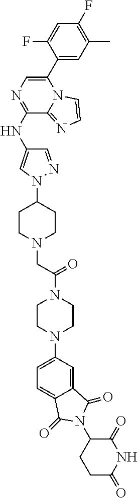 [SMILES: Cc1cc(-c2cnc(Nc3cnn(C4CCN(CC(=O)N5CCN(c6ccc7c(c6)C(=O)N(C6CCC(=O)NC6=O)C7=O)CC5)CC4)c3)c3nccn23)c(F)cc1F] | 5-(4-(2-(4-(4- ((5-(2,4- difluoro-5- methylphenyl) imidazo[1,2- a]pyrazin-8- yl)amino)-1H- pyrazol-1- yl)piperidin-1- yl)acetyl) piperazin-1- yl)-2- (2,6- dioxopiperidin- 3- yl)isoindoline- 1,3-dione | 38.5 mg, 6% yield (2 steps) yellow solid [M + H]⁺ = 792.3 ¹H NMR (400 MHz, DMSO-d₆) δ ppm 1.91-2.10 (m, 5 H), 2.23 (br t, J = 10.9 Hz, 2 H), 2.28 (s, 3 H), 2.54-2.63 (m, 2 H), 2.82-2.91 (m, 1 H), 2.95 (br d, J = 11.2 Hz, 2 H), 3.26 (s, 2 H), 3.49 (br s, 2 H), 3.52- 3.67 (m, 4 H), 3.77 (br s, 2 H), 4.07- 4.21 (m, 1 H), 5.07 (dd, J = 12.8, 5.5 Hz, 1 H), 7.24-7.32 (m, 1 H), 7.38 (s, 1 H), 7.42 (s, 1 H), 7.43- 7.49 (m, 1 H), 7.57-7.65 (m, 3 H), 7.70 (d, J = 8.6 Hz, 1 H), 7.80 (s, 1 H), 8.23 (s, 1 H), 9.98 (s, 1 H), 11.08 (s, 1 H). ¹⁹F NMR (377 MHz, DMSO-d₆) δ ppm −112.17 (d, J = 8.2 Hz, 1 F), −111.25 (d, J = 8.2 Hz, 1 F). |
| TBM-2 | CBM-5 | 10 | 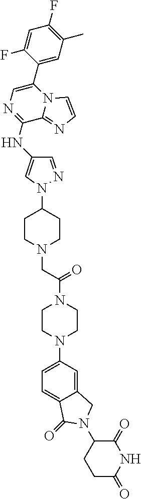 [SMILES: Cc1cc(-c2cnc(Nc3cnn(C4CCN(CC(=O)N5CCN(c6ccc7c(c6)CN(C6CCC(=O)NC6=O)C7=O)CC5)CC4)c3)c3nccn23)c(F)cc1F] | 3-(5-(4-(2-(4- (4-((5-(2,4- difluoro-5- methylphenyl) imidazo[1,2- a]pyrazin-8- yl)amino)-1H- pyrazol-1- yl)piperidin-1- yl)acetyl) piperazin-1- yl)-1- oxoisoindolin- 2- yl)piperidine- 2,6-dione | 72 mg, 6% yield (2 steps), white solid [M + H]⁺ = 778.4 ¹H NMR (400 MHz, DMSO-d₆) δ ppm 1.88-2.06 (m, 5 H), 2.22 (br t, J = 10.9 Hz, 2 H), 2.29 (s, 3 H),2.33- 2.43 (m, 1 H), 2.58 (br d, J = 18.1Hz, 1 H), 2.83-2.92 (m, 1 H), 2.95 (br d, J = 11.7 Hz, 2 H), 3.30 (br s, 3 H), 3.38 (br s, 3 H), 3.62 (br s, 2 H), 3.76 (br s, 2 H), 4.09-4.18 (m, 1 H), 4.21 (dd, J = 16.9, 1.0 Hz, 1 H), 4.34 (dd, J = 17.6, 1.0 Hz, 1 H), 5.05 (dd, J = 13.2, 5.1 Hz, 1 H), 7.07-7.14 (m, 2 H), 7.39 7.48 (m, 2 H), 7.54 (d, J = 8.3 Hz, 1 H), 7.57- 7.65 (m, 3 H), 7.80 (s, 1 H), 8.16 (s, 1 H), 8.23 (s, 1 H), 9.98 (s, 1 H), 10.94 (s, 1 H), 1 H not apparent. ¹⁹F NMR (377 MHz, DMSO-d₆) δ ppm −112.17 (d, J = 9.5 Hz, 1 F), −111.25 (d, J = 8.2 Hz, 1 F). |
| TBM-1 | CBM-7 | 16 | 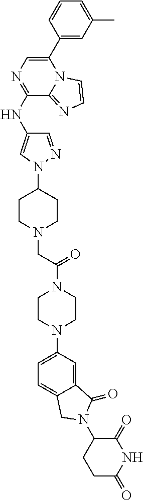 [SMILES: Cc1cccc(-c2cnc(Nc3cnn(C4CCN(CC(=O)N5CCN(c6ccc7c(c6)C(=O)N(C6CCC(=O)NC6=O)C7)CC5)CC4)c3)c3nccn23)c1] | 3-(1-oxo-6-(4- (2-(4-(4-((5- (m- tolyl)imidazo [1,2-a]pyrazin-8- yl)amino)-1H- pyrazol-1- yl)piperidin-1- yl)acetyl) piperazin-1- yl)isoindolin- 2- yl)piperidine- 2,6-dione | 70.4 mg, 31% yield (2 steps), white solid, [M + H]⁺ = 742.4 ¹H NMR (400 MHz, DMSO-d₆) δ ppm 1.91-2.07 (m, 5 H), 2.25 (br t, J = 10.9 Hz, 2 H), 2.32-2.39 (m, 1 H), 2.41 (s, 3 H), 2.54-2.65 (m, 1 H), 2.82-2.93 (m, 1 H), 2.96 (br d, J = 12.0 Hz, 2 H), 3.18 (br s, 2 H), 3.28 (br s, 4 H), 3.63 (br s, 2 H), 3.76 (br s, 2 H), 4.09-4.17 (m, 1 H), 4.20 (d, J = 16.9 Hz, 1 H), 4.33 (d, J = 16.6 Hz, 1 H), 5.10 (dd, J = 13.2, 4.9 Hz, 1 H), 7.22 (d, J = 1.7 Hz, 1 H), 7.28-7.36 (m, 2 H), 7.39, 7.53 (m, 5 H), 7.64 (s, 1 H), 7.80 (s, 1 H), 7.89 (s, 1 H), 8.14 (s, 1 H), 8.23 (s, 1 H), 9.88 (s, 1 H), 10.97 (s, 1 H), 1 H not apparent. |
| TBM-2 | CBM-2 | 17 | 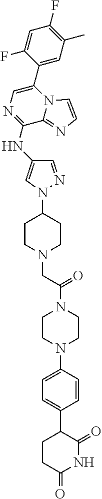 [SMILES: Cc1cc(-c2cnc(Nc3cnn(C4CCN(CC(=O)N5CCN(c6ccc(C7CCC(=O)NC7=O)cc6)CC5)CC4)c3)c3nccn23)c(F)cc1F] | 3-(4-(4-(2-(4- (4-((5-(2,4- difluoro-5- methylphenyl) imidazo[1,2- a]pyrazin-8- yl)amino)-1H- pyrazol-1- yl)piperidin-1- yl)acetyl) piperazin-1- yl)phenyl) piperidine- 2,6-dione | 35 mg, 16% yield ( 2 steps), light- yellow solid [M + H] = 723.3. ¹H NMR (400 MHz, DMSO-d₆) δ ppm 1.88-2.04 (m, 5 H), 2.08- 2.17 (m, 1 H), 2.17-2.26 (m, 2 H), 2.29 (s, 3 H), 2.41-2.48 (m, 1 H), 2.57-2.68 (m, 1 H), 2.90-2.98 (m, 2 H), 3.04-3.14 (m, 2 H), 3.14- 3.21 (m, 2 H), 3.32 (br s, 2 H), 3.52- 3.68 (m, 2 H), 3.68-3.77 (m, 3 H), 4.09-4.19 (m, 1 H), 6.94 (d, J = 8.6 Hz, 2 H), 7.07 (d, J = 8.6 Hz, 2 H), 7.41-7.48 (m, 2 H), 7.58- 7.64 (m, 3 H), 7.80 (s, 1 H), 8.23 (s, 1 H), 9.98 (s, 1 H), 10.77 (s, 1 H). ¹⁹F NMR (377 MHz, DMSO-d₆) δ ppm −112.16 (q, J = 8.2 Hz, 1 F), −111.24 (q, J = 8.2 Hz, 1 F). |
| TBM-2 | CBM-7 | 18 | 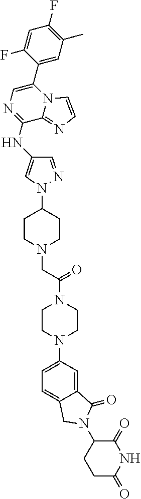 [SMILES: Cc1cc(-c2cnc(Nc3cnn(C4CCN(CC(=O)N5CCN(c6ccc7c(c6)C(=O)N(C6CCC(=O)NC6=O)C7)CC5)CC4)c3)c3nccn23)c(F)cc1F] | 3-(6-(4-(2-(4- (4-((5-(2,4- difluoro-5- methylphenyl) imidazo[1,2- a]pyrazin-8- yl)amino)-1H- pyrazol-1- yl)piperidin-1- yl)acetyl)piper azin-1-yl)-1- oxoisoindolin- 2- yl)piperidine- 2,6-dione | 41 mg, 12% yield (2 steps) white solid [M + H]⁺ = 778.3. ¹H NMR (400 MHz, DMSO-d₆) δ ppm 1.89-2.05 (m, 5 H), 2.18- 2.27 (m, 2 H), 2.29 (s, 3 H), 2.31- 2.43 (m, 1 H), 2.53-2.68 (m, 1 H), 2.85-2.99 (m, 3 H), 3.14-3.20 (m, 2 H), 3.23-3.29 (m, 4 H), 3.59- 3.66 (m, 2 H), 3.73-3.80 (m, 2 H), 4.09-4.18 (m, 1 H), 4.18-4.25 (m, 1 H), 4.30-4.38 (m, 1 H), 5.10 (dd, J = 13.3, 5.0 Hz, 1 H), 7.22 (s, 1 H), 7.31 (dd, J = 8.6, 2.0 Hz, 1 H), 7.41- 7.48 (m, 3 H), 7.58-7.63 (m, 3 H), 7.81 (s, 1 H), 8.23 (s, 1 H), 9.97 (s, 1 H), 10.97 (s, 1 H). ¹⁹F NMR (377 MHz, DMSO-d₆) δ ppm −112.15 (q, J = 8.2 Hz, 1 F), −111.25 (q, J = 8.2 Hz, 1 F). |
| TBM-1 | CBM-5 | 19 | 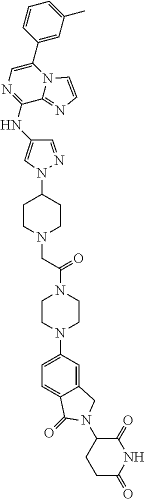 [SMILES: Cc1cccc(-c2cnc(Nc3cnn(C4CCN(CC(=O)N5CCN(c6ccc7c(c6)CN(C6CCC(=O)NC6=O)C7=O)CC5)CC4)c3)c3nccn23)c1] | 3-(1-oxo-5-(4- (2-(4-(4-((5- (m- tolyl)imidazo [1,2-a]pyrazin-8- yl)amino)-1H- pyrazol-1- yl)piperidin-1- yl)acetyl) piperazin-1- yl)isoindolin- 2- yl)piperidine- 2,6-dione | 101 mg, 40% yield (2 steps) [M + H]⁺ = 742.3 ¹H NMR (400 MHz, DMSO-d6 ) 8 ppm 1.91-2.04 (m, 5 H), 2.22 (br t, J = 10.8 Hz, 2 H), 2.29-2.39 (m, 1 H), 2.40 (s, 3 H), 2.55-2.71 (m, 1 H), 2.84-2.99 (m, 3 H), 3.25 (s, 2 H), 3.30 (br s, 2 H), 3.38 (br s, 2 H), 3.62 (br s, 2 H), 3.76 (br s, 2 H), 4.07-4.17 (m, 1 H), 4.19 (d, J = 16.9 Hz, 1 H), 4.34 (d, J = 17.6 Hz, 1 H), 5.05 (dd, J = 13.2, 5.1 Hz, 1 H), 7.08-7.14 (m, 2 H), 7.32 (br d, J = 6.4 Hz, 1 H), 7.41-7.51 (m, 4 H), 7.54 (d, J = 8.3 Hz, 1 H), 7.64 (s, 1 H), 7.80 (s, 1 H), 7.89 (s, 1 H), 8.17 (s, 1 H), 8.24 (s, 1 H), 9.88 (s, 1 H), 10.94 (s, 1 H). |
| TBM-1 | CBM-2 | 20 | 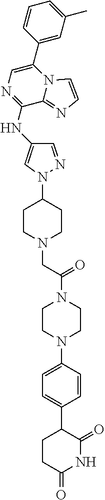 [SMILES: Cc1cccc(-c2cnc(Nc3cnn(C4CCN(CC(=O)N5CCN(c6ccc(C7CCC(=O)NC7=O)cc6)CC5)CC4)c3)c3nccn23)c1] | 3-(4-(4-(2-(4- (4-((5-(m- tolyl)imidazo [1,2-a]pyrazin-8- yl)amino)-1H- pyrazol-1- yl)piperidin-1- yl)acetyl) piperazin-1- yl)phenyl) piperidine-2,6- dione | 28 mg, 13% yield (2 steps) tan solid [M + H]⁺ = 687.4 ¹H NMR (400 MHz, DMSO-d₆) δ ppm 1.88-2.04 (m, 5 H), 2.05- 2.16 (m, 1 H), 2.18-2.26 (m, 2 H), 2.38-2.47 (m, 4 H), 2.58-2.68 (m, 1 H), 2.94 (br d, J = 12.0 Hz, 2 H), 3.06-3.13 (m, 2 H), 3.15-3.21 (m, 2 H), 3.24 (s, 2 H), 3.57-3.65 (m, 2 H), 3.65-3.78 (m, 3 H), 4.09- 4.19 (m, 1 H), 6.94 (br d, J = 8.3 Hz, 2 H), 7.07 (br d, J = 8.1 Hz, 2 H), 7.32 (br d, J = 6.4 Hz, 1 H), 7.40-7.51 (m, 4 H), 7.64 (s, 1 H), 7.79 (s, 1 H), 7.89 (s, 1 H), 8.24 (s, 1 H), 9.88 (s, 1 H), 10.77 (s, 1 H) |
| TBM-4 | CBM-1 | 21 | 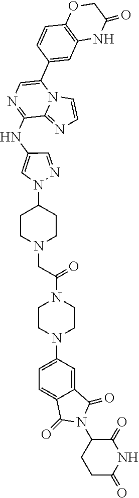 [SMILES: O=C1CCC(N2C(=O)c3ccc(N4CCN(C(=O)CN5CCC(n6cc(Nc7ncc(-c8ccc9c(c8)NC(=O)CO9)n8ccnc78)cn6)CC5)CC4)cc3C2=O)C(=O)N1] | 2-(2,6- dioxopiperidin- 3-yl)-5-(4-(2- (4-(4-((5-(3- ox0-3,4- dihydro-2H- benzo[b][1,4] oxazin-6- yl)imidazo[1,2- a]pyrazin-8- yl)amino)-1H- pyrazol-1- yl)piperidin-1- yl)acetyl) piperazin-1- yl)isoindoline- 1,3-dione | 21 mg, 9% yield (2 steps), yellow solid [M + H]⁺ = 813.3 ¹H NMR (400 MHz, DMSO-d₆) δ ppm 1.89-2.05 (m, 5 H), 2.17- 2.29 (m, 2 H), 2.53-2.64 (m, 2 H), 2.83-2.90 (m, 1 H), 2.91-3.00 (m, 2 H), 3.26 (br s, 2 H), 3.49 (br d, J = 1.0 Hz, 2 H), 3.57 (br d, J = 2.4 Hz, 2 H), 3.61 (br d, J = 5.4 Hz, 2 H), 3.77 (br d, J = 2.2 Hz, 2 H), 4.09-4.19 (m, 1 H), 4.67 (s, 2 H), 5.07 (dd, J = 12.8, 5.3 Hz, 1 H), 7.09-7.18 (m, 2 H), 7.23 (dd, J = 8.3, 1.7 Hz, 1 H), 7.26-7.31 (m, 1 H), 7.35-7.42 (m, 2 H), 7.65 (s, 1 H), 7.70 (d, J = 8.6 Hz, 1 H), 7.79 (s, 1 H), 7.84-7.88 (m, 1 H), 8.23 (s, 1 H), 9.88 (s, 1 H), 10.84 (s, 1 H), 11.08 (s, 1 H). |
| TBM-4 | CBM-2 | 22 | 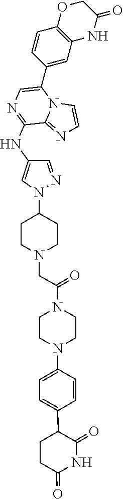 [SMILES: O=C1CCC(c2ccc(N3CCN(C(=O)CN4CCC(n5cc(Nc6ncc(-c7ccc8c(c7)NC(=O)CO8)n7ccnc67)cn5)CC4)CC3)cc2)C(=O)N1] | 3-(4-(4-(2-(4- (4-((5-(3-oxo- 3,4-dihydro- 2H- benzo[b][1,4] oxazin-6- yl)imidazo[1, 2-a]pyrazin-8- yl)amino)-1H- pyrazol-1- yl)piperidin-1- yl)acetyl) piperazin-1- yl)phenyl) piperidine- 2,6-dione | 47 mg, 10% yield (2 steps), off- white solid [M + H]⁺ = 744.3 ¹H NMR (400 MHz, DMSO-d₆) δ ppm 1.88-2.04 (m, 5 H), 2.07- 2.15 (m, 1 H), 2.17-2.28 (m, 2 H), 2.47 (br s, 1 H), 2.62 (td, J = 11.7, 5.9 Hz, 1 H), 2.95 (br d, J = 10.5 Hz, 2 H), 3.09 (br s, 2 H), 3.18 (br s, 2 H), 3.26 (br s, 2 H), 3.57-3.64 (m, 2 H), 3.68-3.78 (m, 3 H), 4.09- 4.22 (m, 1 H), 4.67 (s, 2 H), 6.94 (d, J = 8.6 Hz, 2 H), 7.07 (d, J = 8.6 Hz, 2 H), 7.10-7.18 (m, 2 H), 7.23 (dd, J = 8.2, 2.1 Hz, 1 H), 7.36 (s, 1 H), 7.63-7.68 (m, 1 H), 7.79 (s, 1 H), 7.86 (d, J = 1.0 Hz, 1 H), 8.23 (s, 1 H), 9.88 (s, 1 H), 10.77 (s, 1 H), 10.85 (s, 1 H). |

### Example S21. General Procedure for LDD-J

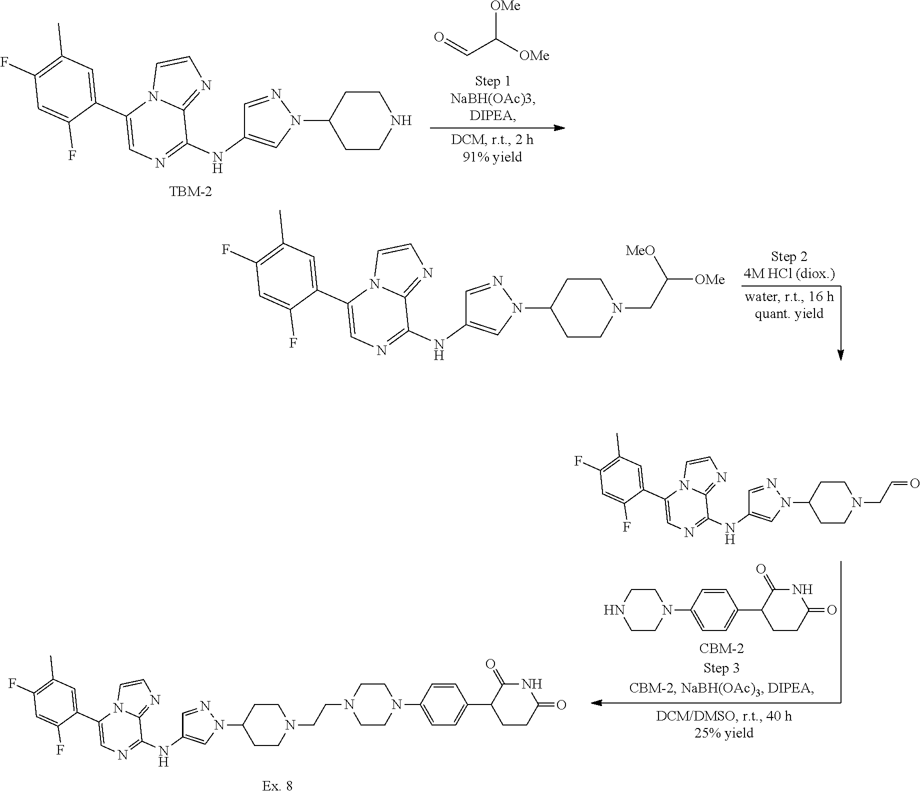 [SMILES: Cc1cc(-c2cnc(Nc3cnn(C4CCNCC4)c3)c3nccn23)c(F)cc1F ; COC(C=O)OC ; COC(CN1CCC(n2cc(Nc3ncc(-c4cc(C)c(F)cc4F)n4ccnc34)cn2)CC1)OC ; Cc1cc(-c2cnc(Nc3cnn(C4CCN(CC=O)CC4)c3)c3nccn23)c(F)cc1F ; O=C1CCC(c2ccc(N3CCNCC3)cc2)C(=O)N1 ; Cc1cc(-c2cnc(Nc3cnn(C4CCN(CCN5CCN(c6ccc(C7CCC(=O)NC7=O)cc6)CC5)CC4)c3)c3nccn23)c(F)cc1F]

Step 1: To a solution of 5-(2,4-difluoro-5-methylphenyl)-N-(1-(piperidin-4-yl)-1H-pyrazol-4-yl)imidazo[1,2-a]pyrazin-8-amine bis hydrochloride TBM-2 (1.0 g, 2.07 mmol, 1 equiv.) in DCM (4.5 mL, 0.42 M) and DMSO (0.40 mL, 0.42 M) were added 2,2-dimethoxyacetaldehyde 60% w/w in H₂O 8 (0.63 mL, 4.14 mmol, 2 equiv.) and NaBH(OAc)₃ (879 mg, 4.15 mmol, 2 equiv.). The reaction mixture was stirred at room temperature for 2 h. The solvent was evaporated and the residue was then purified by reverse phase column chromatography (5% to 100% MeOH in water w/ 0.1% formic acid). The fractions were combined and concentrated to afford 5-(2,4-difluoro-5-methylphenyl)-N-(1-(1-(2,2-dimethoxyethyl)piperidin-4-yl)-1H-pyrazol-4-yl)imidazo[1,2-a]pyrazin-8-amine (936 mg, 91% yield) as a tan solid.

LCMS: [M+H]⁺=498.2.

¹H NMR (400 MHz, DMSO-d₆) δ ppm 1.87-2.02 (m, 4H), 2.18-2.27 (m, 2H), 2.29 (s, 3H), 3.00 (br d, J=11.7 Hz, 2H), 3.27 (s, 6H), 4.08-4.17 (m, 1H), 4.50 (t, J=5.0 Hz, 1H), 7.42-7.48 (m, 2H), 7.58-7.65 (m, 3H), 7.80 (s, 1H), 8.14 (s, 1H), 8.23 (s, 1H), 9.98 (s, 1H).

¹⁹F NMR (377 MHz, DMSO-d₆) δ ppm −112.17 (q, J=9.5 Hz, 1 F), −111.25 (q, J=8.2 Hz, 1 F).

Step 2: To a round-bottom flask was added 5-(2,4-difluoro-5-methylphenyl)-N-(1-(1-(2,2-dimethoxyethyl)piperidin-4-yl)-1H-pyrazol-4-yl)imidazo[1,2-a]pyrazin-8-amine (871 mg, 1.75 mmol, 1 equiv.), 4 M HCl in dioxane (13.14 mL, 52.57 mmol, 30 equiv.) and water (0.48 mL, 4.1 M). The reaction was stirred for 16 h at room temperature. The solvent was removed under reduced pressure and the residue was co-evaporated with MeCN (3×) to give 2-(4-(4-((5-(2,4-difluoro-5-methylphenyl)imidazo[1,2-a]pyrazin-8-yl)amino)-1H-pyrazol-1-yl)piperidin-1-yl)acetaldehyde (880 mg) as a yellow solid as an HCl salt.

LCMS: [M+H₂O+H]⁺=470.2.

¹H NMR (400 MHz, DMSO-d₆) δ ppm 2.20-2.41 (m, 7H), 3.29-3.42 (m, 4H), 3.60-3.76 (m, 2H), 4.35-4.62 (m, 1H), 4.87-5.03 (m, 1H), 7.45-7.54 (m, 2H), 7.61 (t, J=8.2 Hz, 1H), 7.74-7.80 (m, 1H), 7.82 (d, J=4.6 Hz, 2H), 8.24 (s, 1H), 9.92-10.11 (m, 1H).

¹⁹F NMR (377 MHz, DMSO-d₆) δ ppm −112.07 (s, 1 F), −110.60 (s, 1 F).

Step 3: To a solution of 2-(4-(4-((5-(2,4-difluoro-5-methylphenyl)imidazo[1,2-a]pyrazin-8-yl)amino)-1H-pyrazol-1-yl)piperidin-1-yl)acetaldehyde hydrochloride (100 mg, 0.20 mmol, 1 equiv.), 3-(4-piperazin-1-ylphenyl)piperidine-2,6-dione CBM-2 (56 mg, 0.20 mmol, 1 equiv.) and DIPEA (0.25 mL, 1.43 mmol, 7 equiv.) in DCM (0.73 mL, 0.14 M) and DMSO (0.73 mL, 0.14M) under N₂ atm was added NaBH(OAc)₃ (87 mg, 0.4100 mmol, 2 equiv.). The reaction mixture was stirred at room temperature for 40 h. The solvent was evaporated and the residue was then purified by reverse phase column chromatography (5% to 95% MeCN in water w/ 0.1% formic acid). The fractions were combined and concentrated to afford 3-(4-(4-(2-(4-(4-((5-(2,4-difluoro-5-methylphenyl)imidazo[1,2-a]pyrazin-8-yl)amino)-1H-pyrazol-1-yl)piperidin-1-yl)ethyl)piperazin-1-yl)phenyl)piperidine-2,6-dione Ex. 8 (37 mg, 25% yield) as a white solid.

LCMS: [M+H]⁺=709.3.

¹H NMR (400 MHz, DMSO-d₆) δ ppm 1.86-2.05 (m, 5H), 2.09-2.18 (m, 3H), 2.29 (s, 3H), 2.42-2.48 (m, 1H), 2.54-2.69 (m, 6H), 3.01 (br d, J=11.2 Hz, 2H), 3.11 (br s, 4H), 3.72 (dd, J=11.2, 5.1 Hz, 1H), 4.08-4.18 (m, 1H), 6.89 (d, J=8.6 Hz, 2H), 7.04 (d, J=8.6 Hz, 2H), 7.41-7.48 (m, 2H), 7.58-7.65 (m, 3H), 7.80 (s, 1H), 8.23 (s, 1H), 9.98 (s, 1H), 10.77 (s, 1H).

¹⁹F NMR (377 MHz, DMSO-d₆) δ ppm −112.16 (q, J=8.6 Hz, 1 F), −111.26 (q, J=10.0 Hz, 1 F).

TABLE 36
List of Examples prepared by procedure LDD-J

Components

| TBM | CBM | Example | | | |
|---|---|---|---|---|---|
| # | # | Cmp | Structure | IUPAC Name | Characterization |
| TBM-1 | CBM-1 | 2 | 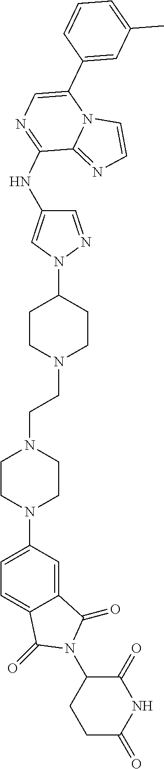 [SMILES: Cc1cccc(-c2cnc(Nc3cnn(C4CCN(CCN5CCN(c6ccc7c(c6)C(=O)N(C6CCC(=O)NC6=O)C7=O)CC5)CC4)c3)c3nccn23)c1] | 2-(2,6- dioxopiperidin-3- yl)-5-(4-(2-(4-(4- ((5-(m- tolyl)imidazo[1,2- a]pyrazin-8- yl)amino)-1H- pyrazol-1- yl)piperidin-1- yl)ethyl)piperazin- 1-yl)isoindoline- 1,3-dione | 28 mg, 26% yield (3 steps), yellow solid [M + H]]⁺ = 742.3 ¹H NMR (400 MHz, DMSO- d6) δ ppm 1.84-2.07 (m, 6 H), 2.09-2.20 (m, 2 H), 2.41 (s, 3 H), 2.53-2.63 (m, 7 H), 2.82-2.94 (m, 1 H), 2.98-3.05 (m, 2 H), 3.41- 3.48 (m, 4 H), 4.08-4.19 (m, 1 H), 5.07 (dd, J = 12.8, 5.3 Hz, 1 H), 7.24-7.28 (m, 1 H), 7.30-7.36 (m, 2 H), 7.42-7.52 (m, 4 H), 7.64 (s, 1 H), 7.68 (d, J = 8.6 Hz, 1 H), 7.80 (s, 1 H), 7.89 (s, 1 H), 8.24 (s, 1 H), 9.88 (s, 1 H), 11.07 (s, 1 H). |
| TBM-1 | CBM-2 | 3 | 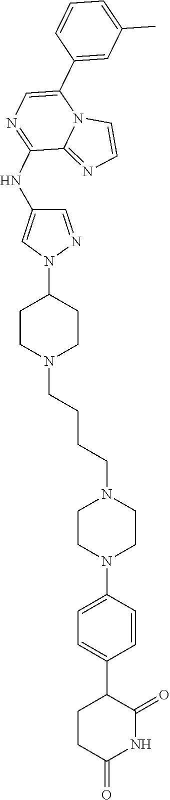 [SMILES: Cc1cccc(-c2cnc(Nc3cnn(C4CCN(CCCCN5CCN(c6ccc(C7CCC(=O)NC7=O)cc6)CC5)CC4)c3)c3nccn23)c1] | 3-(4-(4-(4-(4-(4- ((5-(m- tolyl)imidazo[1,2- a]pyrazin-8- yl)amino)-1H- pyrazol-1- yl)piperidin-1- yl)butyl)piperazin- 1- yl)phenyl) piperidine-2,6- dione | 71 mg, 30% yield (3 steps) white solid [M + H]]⁺ = 701.4 ¹H NMR (400 MHz, DMSO- d6) δ ppm 1.49 (br s, 4 H), 1.84-2.20 (m, 9 H), 2.28- 2.48 (m, 8 H), 2.55-2.70 (m, 2 H), 3.01 (br d, J = 11.2 Hz, 2 H), 3.07-3.17 (m, 5 H), 3.72 (dd, J = 11.2, 4.9 Hz, 1 H), 4.09-4.24 (m, 1 H), 6.89 (d, J = 8.6 Hz, 2 H), 7.04 (d, J = 8.6 Hz, 2 H), 7.32 (br d, J = 6.1 Hz, 1 H), 7.40-7.53 (m, 4 H), 7.61- 7.68 (m, 1 H), 7.80 (s, 1 H), 7.89 (d, J = 1.0 Hz, 1 H), 8.24 (s, 1 H), 9.88 (s, 1 H), 10.76 (s, 1 H). |
| TBM-1 | CBM-2 | 4 | 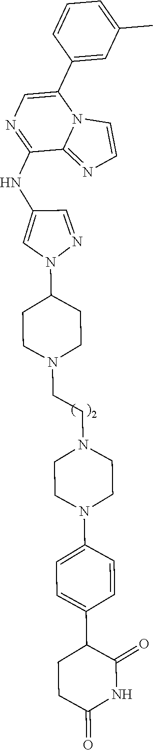 | 3-(4-(4-(3-(4-(4- ((5-(m- tolyl)imidazo[1,2- a]pyrazin-8- yl)amino)-1H- pyrazol-1- yl)piperidin-1- yl)propyl) piperazin-1- yl)phenyl) piperidine-2,6- dione | 14 mg, 3% yield (3 steps), white solid [M + H]]⁺ = 687.4. ¹H NMR (400 MHz, DMSO- d6) δ ppm 1.65 (quin, J = 6.8 Hz, 2 H), 1.87-2.05 (m, 6 H), 2.05-2.21 (m, 3 H), 2.32-2.45 (m, 8 H), 2.56- 2.65 (m, 2 H), 2.99 (br d, J = 11.4 Hz, 2 H), 3.09-3.14 (m, 4 H), 3.72 (dd, J = 11.2, 4.8 Hz, 1 H), 4.08-4.19 (m, 1 H), 6.89 (d, J = 8.6 Hz, 2 H), 7.05 (d, J = 8.4 Hz, 2 H), 7.32 (d, J = 6.7 Hz, 1 H), 7.39-7.56 (m, 4 H), 7.64 (s, 1 H), 7.80 (s, 1 H), 7.89 (s, 1 H), 8.24 (s, 1 H), 9.89 (s, 1 H), 10.77 (s, 1 H). |
| TBM-1 | CBM-2 | 5 | 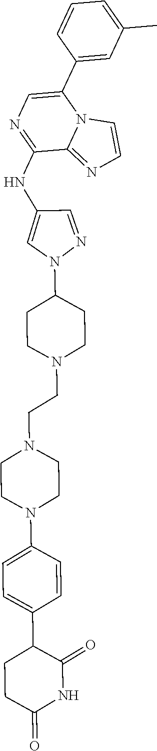 [SMILES: Cc1cccc(-c2cnc(Nc3cnn(C4CCN(CCN5CCN(c6ccc(C7CCC(=O)NC7=O)cc6)CC5)CC4)c3)c3nccn23)c1] | 3-(4-(4-(2-(4-(4- ((5-(m- tolyl)imidazo[1,2- a]pyrazin-8- yl)amino)-1H- pyrazol-1- yl)piperidin-1- yl)ethyl)piperazin- 1- yl)phenyl) piperidine-2,6- dione | 34 mg, 4% yield (3 steps), white solid [M + H]]⁺ = 673.3 ¹H NMR (400 MHz, DMSO- d6) δ ppm 1.87-2.04 (m, 6 H), 2.09-2.16 (m, 3 H), 2.41 (s, 3 H), 2.42-2.45 (m, 1 H), 2.54-2.59 (m, 5 H), 2.60-2.70 (m, 2 H), 3.00 (br d, J = 10.8 Hz, 2 H), 3.11 (br s, 5 H), 3.72 (dd, J = 11.1, 5.0 Hz, 1 H), 4.07-4.18 (m, 1 H), 6.89 (d, J = 8.6 Hz, 2 H), 7.04 (d, J = 8.6 Hz, 2 H), 7.32 (br d, J = 6.6 Hz, 1 H), 7.42 (s, 1 H), 7.45-7.51 (m, 3 H), 7.64 (s, 1 H), 7.80 (s, 1 H), 7.89 (s, 1 H), 8.24 (s, 1 H), 9.88 (s, 1 H), 10.77 (s, 1 H). |
| TBM-2 | CBM-3 | 7 | 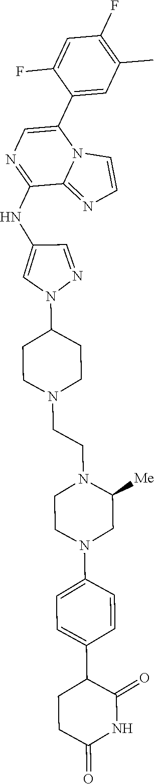 [SMILES: Cc1cc(-c2cnc(Nc3cnn(C4CCN(CCN5CCN(c6ccc(C7CCC(=O)NC7=O)cc6)C[C@@H]5C)CC4)c3)c3nccn23)c(F)cc1F] | 3-(4-((S)-4-(2-(4- (4-((5-(2,4- difluoro-5- methylphenyl) imdazo [1,2-a]pyrazin- 8-yl)amino)-1H- pyrazol-1- yl)piperidin-1- yl)ethyl)-3- methylpiperazin-1- yl)phenyl) piperidine- 2,6-dione | 30 mg, 16% yield (3 steps), white solid [M + H]]⁺ = 723.4. ¹H NMR (400 MHz, DMSO- d6) δ ppm 1.08 (d, J = 5.6 Hz, 3 H), 1.86-2.04 (m, 5 H), 2.07-2.20 (m, 3 H), 2.29 (s, 3 H), 2.34-2.48 (m, 5 H), 2.52-2.56 (m, 1 H), 2.58-2.68 (m, 1 H), 2.74- 2.90 (m, 2 H), 2.91-3.06 (m, 3 H), 3.41 (br d, J = 10.3 Hz, 3 H), 3.72 (dd, J = 10.9, 5.0 Hz, 1 H), 4.08-4.18 (m, 1 H), 6.88 (d, J = 8.8 Hz, 2 H), 7.04 (d, J = 8.6 Hz, 2 H), 7.41-7.49 (m, 2 H), 7.57- 7.64 (m, 3 H), 7.80 (s, 1 H), 8.23 (s, 1H), 9.98 (s, 1 H), 10.77 (s, 1 H). ¹⁹F NMR (377 MHz, DMSO-d6) 8 ppm −112.16 (dd, J = 18.9, 8.2 Hz, 1 F), −111.26 (dd, J = 18.9, 8.2 Hz, 1F). |
| TBM-2 | CBM-2 | 8 | 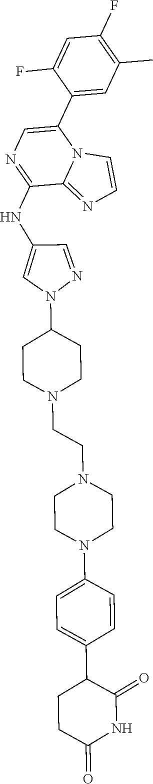 [SMILES: Cc1cc(-c2cnc(Nc3cnn(C4CCN(CCN5CCN(c6ccc(C7CCC(=O)NC7=O)cc6)CC5)CC4)c3)c3nccn23)c(F)cc1F] | 3-(4-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1- yl)phenyl) piperidine- 2,6-dione | 37 mg, 23% yield (3 steps), white solid [M + H]]⁺ = 709.3. ¹H NMR (400 MHz, DMSO- d6) δ ppm 1.86-2.05 (m, 5 H), 2.09-2.18 (m, 3 H), 2.29 (s, 3 H), 2.42-2.48 (m, 1 H), 2.54-2.69 (m, 6 H), 3.01 (br d, J = 11.2 Hz, 2 H), 3.11 (br s, 4 H), 3.72 (dd, J = 11.2, 5.1 Hz, 1 H), 4.08- 4.18 (m, 1 H), 6.89 (d, J = 8.6 Hz, 2 H), 7.04 (d, J = 8.6 Hz, 2 H), 7.41-7.48 (m, 2 H), 7.58-7.65 (m, 3 H), 7.80 (s, 1 H), 8.23 (s, 1 H), 9.98 (s, 1 H), 10.77 (s, 1 H). ¹⁹F NMR (377 MHz, DMSO-d6) δ ppm −112.16 (q, J = 8.6 Hz, 1 F), −111.26 (q, J = 10.0 Hz, 1 F). |
| TBM-2 | CBM-4 | 9 | 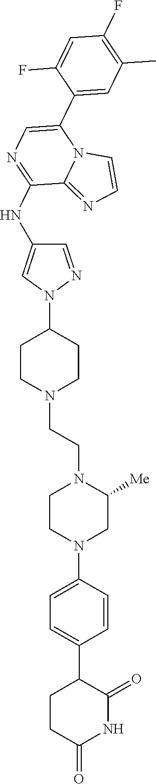 [SMILES: Cc1cc(-c2cnc(Nc3cnn(C4CCN(CCN5CCN(c6ccc(C7CCC(=O)NC7=O)cc6)C[C@H]5C)CC4)c3)c3nccn23)c(F)cc1F] | 3-(4-((R)-4-(2-(4- (4-((5-(2,4- difluoro-5- methylphenyl) imdazo [1,2-a]pyrazin- 8-yl)amino)-1H- pyrazol-1- yl)piperidin-1- yl)ethyl)-3- methylpiperazin-1- yl)phenyl) piperidine- 2,6-dione | 42 mg, 10% yield (3 steps), white solid [M + H]]⁺ = 723.3. ¹H NMR (400 MHz, DMSO- d6) δ ppm 1.08 (d, J = 5.9 Hz, 3 H), 1.87-2.05 (m, 6 H), 2.07-2.19 (m, 3 H), 2.29 (s, 3 H), 2.34-2.46 (m, 3 H), 2.58-2.69 (m, 1 H), 2.74-2.91 (m, 3 H), 2.91- 3.07 (m, 4 H), 3.38-3.45 (m, 3 H), 3.72 (dd, J = 10.9, 4.8 Hz, 1 H), 4.08-4.18 (m, 1 H), 6.88 (d, J = 8.6 Hz, 2 H), 7.04 (d, J = 8.6 Hz, 2 H), 7.42-7.48 (m, 2 H), 7.58- 7.64 (m, 3 H), 7.80 (s, 1 H), 8.16 (s, 1 H), 8.23 (s, 1 H), 9.98 (s, 1 H), 10.77 (s, 1 H). ¹⁹F NMR (377 MHz, DMSO-d6) δ ppm −112.16 (q, J = 8.6 Hz, 1 F), −111.26 (q, J = 8.2 Hz, 1 F). |
| TBM-3 | CBM-2 | 12 | 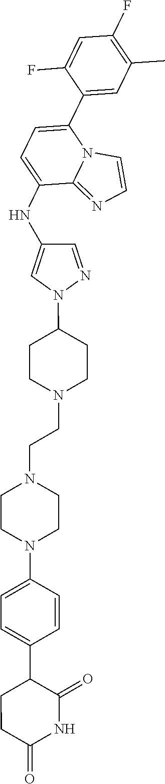 [SMILES: Cc1cc(-c2ccc(Nc3cnn(C4CCN(CCN5CCN(c6ccc(C7CCC(=O)NC7=O)cc6)CC5)CC4)c3)c3nccn23)c(F)cc1F] | 3-(4-(4-(2-(4-(4- ((5-(2,4-difluoro- 5- methylphenyl) imdazo [1,2-a]pyridin- 8-yl)amino)-1H- pyrazol-1- yl)piperidin-1- yl)ethyl)piperazin- 1- yl)phenyl) piperidine- 2,6-dione | 35 mg, 13% yield (3 steps), off-white solid, [M + H]]⁺ = 708.3 ¹H NMR (400 MHz, DMSO- d6 ) δ ppm 1.91-2.04 (m, 5 H), 2.06-2.18 (m, 3 H), 2.28 (s, 3 H), 2.41-2.47 (m, 1 H), 2.53-2.68 (m, 6 H), 2.96-3.07 (m, 2 H), 3.11 (s, 4 H), 3.72 (dd, J = 10.8, 4.9 Hz, 1 H), 4.06-4.16 (m, 1 H), 6.53 (d, J = 7.8 Hz, 1 H), 6.74 (d, J = 7.8 Hz, 1 H), 6.89 (d, J = 8.8 Hz, 2 H), 7.05 (d, J = 8.6 Hz, 2 H), 7.41 (t, J = 10.1 Hz, 1 H), 7.46 (d, J = 2.0 Hz, 1 H), 7.50-7.57 (m, 3 H), 7.91 (s, 1 H), 8.12 (s, 1 H), 10.77 (s, 1 H). |
| TBM-2 | CBM-6 | 13 | 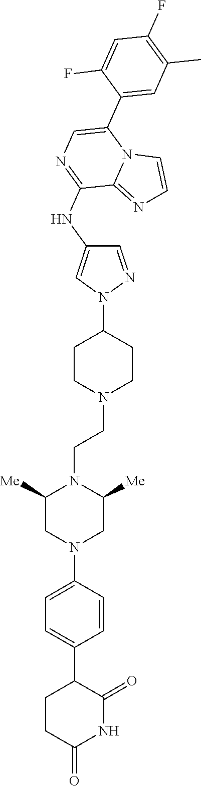 [SMILES: Cc1cc(-c2cnc(Nc3cnn(C4CCN(CCN5[C@H](C)CN(c6ccc(C7CCC(=O)NC7=O)cc6)C[C@@H]5C)CC4)c3)c3nccn23)c(F)cc1F] | 3-(4-((3S,5R)-4- (2-(4-(4-((5-(2,4- difluoro-5- methylphenyl) imdazo [1,2-a]pyrazin- 8-yl)amino)-1H- pyrazol-1- yl)piperidin-1- yl)ethyl)-3,5- dimethylpiperazin- 1- yl)phenyl) piperidine- 2,6-dione | 14 mg, 3% yield (3 steps) [M + H]]⁺ = 737.4. ¹H NMR (400 MHz, DMSO- d6) δ ppm 1.09 (d, J = 6.1 Hz, 6 H), 1.88-2.03 (m, 5 H), 2.09-2.18 (m, 3 H), 2.29 (s, 5 H), 2.37-2.45 (m, 2 H), 2.58-2.65 (m, 1 H), 2.66-2.73 (m, 2 H), 2.75- 2.81 (m, 2 H), 2.98 (br. d, J = 11.2 Hz, 2 H), 3.52 (br. d, J = 10.5 Hz, 3 H), 3.71 (br. dd, J = 10.8, 4.9 Hz, 1 H), 4.13 (br. s, 1 H), 6.88 (br. d, J = 8.8 Hz, 2 H), 7.03 (d, J = 8.8 Hz, 2 H), 7.43 (s, 2 H), 7.57-7.66 (m, 3 H), 7.80 (s, 1 H), 8.23 (s, 3 H), 9.98 (s, 1 H), 10.73-10.81 (m, 1 H). ¹⁹F NMR (377 MHz, DMSO-d6) δ ppm −112.17 (d, J = 8.2 Hz, 1 F), −111.25 (d, J = 8.2 Hz, 1 F). |
| TBM-4 | CBM-2 | 15 | 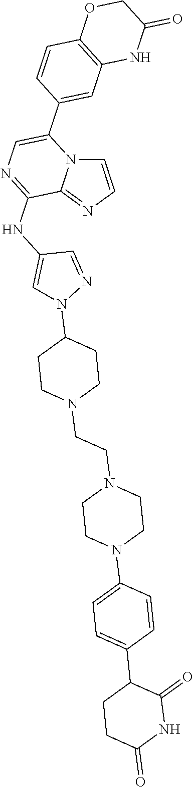 [SMILES: O=C1CCC(c2ccc(N3CCN(CCN4CCC(n5cc(Nc6ncc(-c7ccc8c(c7)NC(=O)CO8)n7ccnc67)cn5)CC4)CC3)cc2)C(=O)N1] | 3-(4-(4-(2-(4-(4- ((5-(3-oxo-3,4- dihydro-2H- benzo[b][1,4] oxazin-6- yl)imidazo[1,2- a]pyrazin-8- yl)amino)-1H- pyrazol-1- yl)piperidin-1- yl)ethyl)piperazin- 1- yl)phenyl) piperidine- 2,6-dione | 23 mg, 12% yield (3 steps), white solid [M + H]]⁺ = 730.4 ¹H NMR (400 MHz, DMSO- d6) δ ppm 1.85-2.05 (m, 6 H), 2.06-2.21 (m, 3 H), 2.44 (br d, J = 4.4 Hz, 1 H), 2.53-2.69 (m, 6 H), 3.02 (br d, J = 11.7 Hz, 2 H), 3.11 (br d, J = 4.9 Hz, 4 H), 3.72 (dd, J = 11.1, 5.0 Hz, 1 H), 4.13 (br s, 1 H), 4.67 (s, 2 H), 6.89 (d, J = 8.8 Hz, 2 H), 7.05 (d, J = 8.8 Hz, 2 H), 7.10-7.17 (m, 2 H), 7.21- 7.26 (m, 1 H), 7.37 (s, 1 H), 7.63-7.68 (m, 1 H), 7.79 (s, 1 H), 7.86 (d, J = 1.0 Hz, 1 H), 8.15 (s, 1 H), 8.24 (s, 1 H), 9.88 (s, 1 H), 10.77 (s, 1 H), 10.84 (s, 1 H). |
| TBM-2 | CBM-8 | 25 | 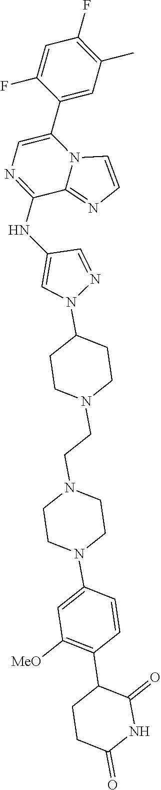 [SMILES: COc1cc(N2CCN(CCN3CCC(n4cc(Nc5ncc(-c6cc(C)c(F)cc6F)n6ccnc56)cn4)CC3)CC2)ccc1C1CCC(=O)NC1=O] | 3-(4-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1-yl)-2- methoxyphenyl) piperidine- 2,6-dione | 30 mg, 16% yield (3 steps), white solid [M + H]]⁺ = 739.4 ¹H NMR (400 MHz, DMSO- d6) δ ppm 1.86 (dt, J = 8.8, 4.4 Hz, 1 H), 1.91-2.04 (m, 4 H), 2.08-2.26 (m, 3 H), 2.29 (s, 3 H), 2.39-2.48 (m, 1 H), 2.53-2.69 (m, 9 H), 2.99-3.10 (m, 2 H), 3.14 (br s, 4 H), 3.72 (s, 3 H), 3.78 (dd, J = 11.4, 5.0 Hz, 1 H), 4.10-4.21 (m, 1 H), 6.45 (br d, J = 8.6 Hz, 1 H), 6.55 (s, 1 H), 6.92 (d, J = 8.3 Hz, 1 H), 7.42-7.48 (m, 2 H), 7.58- 7.64 (m, 3 H), 7.81 (s, 1 H), 8.24 (s, 1 H), 9.98 (s, 1 H), 10.67 (s, 1 H). ¹⁹F NMR (377 MHz, DMSO-d6) δ ppm −112.25- −112.07 (m, 1 F), −111.32- −111.15 (m, 1 F) |
| TBM-5 | CBM-2 | 26 | 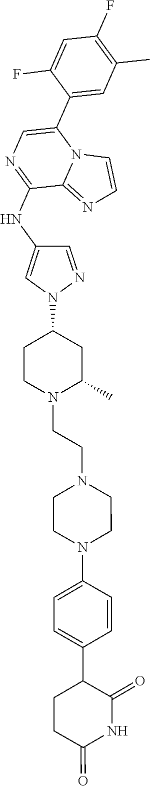 [SMILES: Cc1cc(-c2cnc(Nc3cnn([C@H]4CCN(CCN5CCN(c6ccc(C7CCC(=O)NC7=O)cc6)CC5)[C@@H](C)C4)c3)c3nccn23)c(F)cc1F] | 3-(4-(4-(2- ((2S,4S)-4-(4-((5- (2,4-difluoro-5- methylphenyl) imdazo [1,2-a]pyrazin- 8-yl)amino)-1H- pyrazol-1-yl)-2- methylpiperidin-1- yl)ethyl)piperazin- 1- yl)phenyl) piperidine- 2,6-dione | 59 mg, 31% yield (3 steps), white solid [M + H]]⁺ = 723.3 ¹H NMR (400 MHz, DMSO- d6) δ ppm 1.12 (d, J = 6.1 Hz, 3 H), 1.62 (q, J = 11.7 Hz, 1 H), 1.81-1.93 (m, 1 H), 1.94-2.04 (m, 3 H), 2.06-2.19 (m, 1 H), 2.29 (s, 3 H), 2.31-2.48 (m, 6 H), 2.53-2.68 (m, 5 H), 2.87- 2.96 (m, 1 H), 3.04-3.17 (m, 5 H), 3.72 (dd, J = 10.8, 4.9 Hz, 1 H), 4.11-4.23 (m, 1 H), 6.89 (d, J = 8.8 Hz, 2 H), 7.05 (d, J = 8.8 Hz, 2 H), 7.42-7.48 (m, 2 H), 7.58- 7.64 (m, 3 H), 7.79 (s, 1 H), 8.22 (s, 1 H), 9.98 (s, 1 H), 10.77 (s, 1 H). ¹⁹F NMR (377 MHz, DMSO-d6) δ ppm −112.21- −112.10 (m, 1 F), −111.32- −111.18 (m, 1 F). |
| TBM-6 | CBM-4 | 29 | 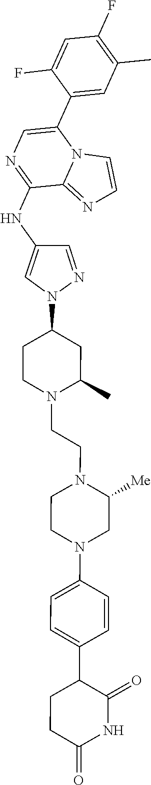 [SMILES: Cc1cc(-c2cnc(Nc3cnn([C@@H]4CCN(CCN5CCN(c6ccc(C7CCC(=O)NC7=O)cc6)C[C@H]5C)[C@H](C)C4)c3)c3nccn23)c(F)cc1F] | 3-(4-((R)-4-(2- ((2R,4R)-4-(4-((5- (2,4-difluoro-5- methylphenyl) imdazo [1,2-a]pyrazin- 8-yl)amino)-1H- pyrazol-1-yl)-2- methylpiperidin-1- yl)ethyl)-3- methylpiperazin-1- yl)phenyl) piperidine- 2,6-dione | 92 mg, 40% yield (3 steps), off-white solid [M + H]]⁺ = 737.4; [M + 2H]2⁺ = 369.3 ¹H NMR (400 MHz, DMSO- d6) δ ppm 1.11 (d, J = 5.4 Hz, 3 H), 1.16 (d, J = 5.9 Hz, 3 H), 1.61-1.82 (m, 1 H), 1.91-2.05 (m, 4 H), 2.07- 2.21 (m, 1 H), 2.29 (s, 3 H), 2.39-2.46 (m, J = 4.4 Hz, 2 H), 2.53-2.71 (m, 5 H), 2.77-3.03 (m, 4 H), 3.09- 3.22 (m, 1 H), 3.42 (br d, J = 9.3 Hz, 3 H), 3.72 (dd, J = 10.9, 4.8 Hz, 1 H), 4.18- 4.32 (m, 1 H), 6.89 (d, J = 8.6 Hz, 2 H), 7.05 (d, J = 8.6 Hz, 2 H), 7.40-7.50 (m, 2 H), 7.58-7.63 (m, 3 H), 7.80 (s, 1 H), 8.23 (s, 1 H), 9.99 (s, 1 H), 10.77 (s, 1 H). ¹⁹F NMR (377 MHz, DMSO-d6) δ ppm −112.30- −112.09 (m, 1 F), −111.37- −111.12 (m, 1 F). |
| TBM-6 | CBM-2 | 30 | 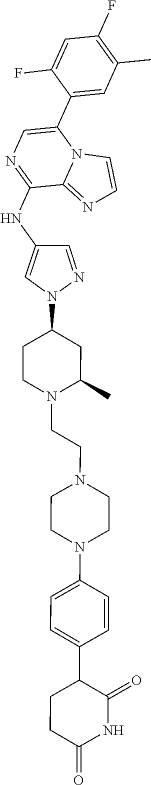 [SMILES: Cc1cc(-c2cnc(Nc3cnn([C@@H]4CCN(CCN5CCN(c6ccc(C7CCC(=O)NC7=O)cc6)CC5)[C@H](C)C4)c3)c3nccn23)c(F)cc1F] | 3-(4-(4-(2- ((2R,4R)-4-(4-((5- (2,4-difluoro-5- methylphenyl) imdazo [1,2-a]pyrazin- 8-yl)amino)-1H- pyrazol-1-yl)-2- methylpiperidin-1- yl)ethyl)piperazin- 1- yl)phenyl) piperidine- 2,6-dione | 76 mg, 34% yield (3 steps), white solid [M + H]]⁺ = 723.3; [M + 2H]2⁺ = 362.3 ¹H NMR (400 MHz, DMSO- d6) δ ppm 1.12 (d, J = 5.9 Hz, 3 H), 1.62 (q, J = 11.9 Hz, 1 H), 1.81-1.93 (m, 1 H), 1.95-2.04 (m, 3 H), 2.06-2.19 (m, 1 H), 2.29 (s, 3 H), 2.31-2.47 (m, 5 H), 2.53-2.70 (m, 6 H), 2.85- 2.96 (m, 1 H), 3.04-3.14 (m, 5 H), 3.72 (dd, J = 11.0, 4.9 Hz, 1 H), 4.11-4.25 (m, 1 H), 6.89 (d, J = 8.6 Hz, 2 H), 7.05 (d, J = 8.6 Hz, 2 H), 7.42-7.48 (m, 2 H), 7.58- 7.63 (m, 3 H), 7.79 (s, 1 H), 8.22 (s, 1 H), 9.97 (s, 1 H), 10.76 (s, 1 H). ¹⁹F NMR (377 MHz, DMSO-d6) δ ppm −112.25- −112.05 (m, 1 F), −111.39- −111.13 (m, 1 F). |
| TBM-2 | CBM-9 | 31 | 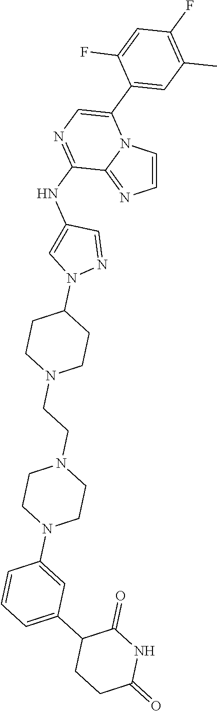 [SMILES: Cc1cc(-c2cnc(Nc3cnn(C4CCN(CCN5CCN(c6cccc(C7CCC(=O)NC7=O)c6)CC5)CC4)c3)c3nccn23)c(F)cc1F] | 3-(3-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1- yl)phenyl) piperidine- 2,6-dione | 51 mg, 26% yield (3 steps), white solid [M + H]]⁺ = 709.3 ¹H NMR (400 MHz, DMSO- d6) δ ppm 1.86-2.24 (m, 9 H), 2.29 (s, 3 H), 2.42-2.48 (m, 2 H), 2.52-2.69 (m, 6 H), 3.00 (br d, J = 11.2 Hz, 2 H), 3.07-3.16 (m, 4 H), 3.75 (dd, J = 11.2, 4.9 Hz, 1 H), 4.06-4.20 (m, 1 H), 6.59-6.65 (m, 1 H), 6.76- 6.86 (m, 2 H), 7.15 (t, J = 7.9 Hz, 1 H), 7.41-7.49 (m, 2 H), 7.58-7.65 (m, 3 H), 7.80 (s, 1 H), 8.19 (s, 1 H), 8.23 (s, 1 H), 9.98 (s, 1 H), 10.79 (s, 1 H). ¹⁹F NMR (377 MHz, DMSO-d6) 8 ppm −112.16 (q, J = 9.5 Hz, 1 F), −111.31-−111.20 (m, 1 F). |
| TBM-7 | CBM-4 | 32 | 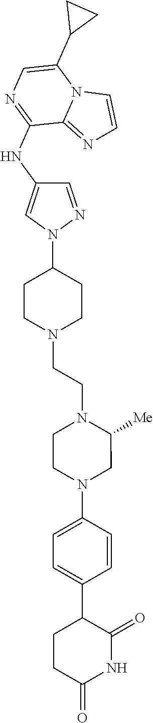 [SMILES: C[C@@H]1CN(c2ccc(C3CCC(=O)NC3=O)cc2)CCN1CCN1CCC(n2cc(Nc3ncc(C4CC4)n4ccnc34)cn2)CC1] | 3-(4-((R)-4-(2-(4- (4-((5- cyclopropylimidaz o[1,2-a]pyrazin-8- yl)amino)-1H- pyrazol-1- yl)piperidin-1- yl)ethyl)-3- methylpiperazin-1- yl)phenyl) piperidine- 2,6-dione | 9 mg, 14% yield (3 steps), white solid [M + H]]⁺ = 637.4; [M + 2H]2⁺ = 319.2. ¹H NMR (400 MHz, DMSO- d6) δ ppm 0.70-0.76 (m, 2 H), 0.96-1.04 (m, 2 H), 1.08 (d, J = 5.6 Hz, 3 H), 1.82-2.06 (m, 6 H), 2.06- 2.19 (m, 3 H), 2.32-2.48 (m, 5 H), 2.57-2.68 (m, 1 H), 2.73-2.89 (m, 2 H), 2.90-3.05 (m, 3 H), 3.41 (br d, J = 10.5 Hz, 2 H), 3.72 (dd, J = 10.9, 5.0 Hz, 1 H), 4.09 (tt, J = 11.3, 4.7 Hz, 1 H), 6.88 (d, J = 8.6 Hz, 2 H), 7.04 (d, J = 8.8 Hz, 2 H), 7.22 (d, J = 1.2 Hz, 1 H), 7.65 (d, J = 1.0 Hz, 1 H), 7.74 (s, 1 H), 8.05 (d, J = 1.0 Hz, 1 H), 8.17 (s, 1 H), 9.64 (s, 1 H), 10.77 (s, 1 H). |
| TBM-8 | CBM-2 | 33 | 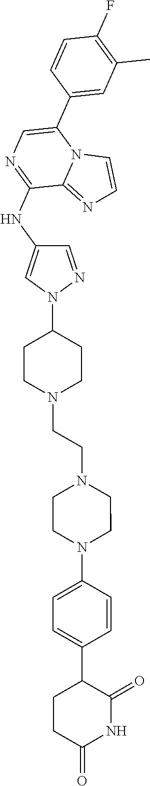 [SMILES: Cc1cc(-c2cnc(Nc3cnn(C4CCN(CCN5CCN(c6ccc(C7CCC(=O)NC7=O)cc6)CC5)CC4)c3)c3nccn23)ccc1F] | 3-(4-(4-(2-(4-(4- ((5-(4-fluoro-3- methylphenyl) imdazo [1,2-a]pyrazin- 8-yl)amino)-1H- pyrazol-1- yl)piperidin-1- yl)ethyl)piperazin- 1- yl)phenyl) piperidine- 2,6-dione | 38 mg, 16% yield (3 steps), white solid, [M + H]]⁺ = 691.4; [M + 2H]2⁺ = 346.2. ¹H NMR (400 MHz, DMSO- d6) δ ppm 1.88-2.05 (m, 5 H), 2.07-2.25 (m, 3 H), 2.33 (d, J = 1.5 Hz, 3 H), 2.41-2.48 (m, 1 H), 2.52- 2.68 (m, 9 H), 3.04 (br d, J = 11.5 Hz, 2 H), 3.09-3.15 (m, 4 H), 3.72 (dd, J = 11.0, 4.9 Hz, 1 H), 4.09-4.20 (m, 1 H), 6.89 (d, J = 8.8 Hz, 2 H), 7.05 (d, J = 8.6 Hz, 2 H), 7.33 (t, J = 9.3 Hz, 1 H), 7.40 (s, 1 H), 7.53 (ddd, J = 8.1, 5.3, 2.3 Hz, 1 H), 7.62 (dd, J = 7.3, 2.0 Hz, 1 H), 7.64 (d, J = 1.0 Hz, 1 H), 7.80 (s, 1 H), 7.87 (d, J = 1.2 Hz, 1 H), 8.15 (s, 1 H), 8.24 (s, 1 H), 9.89 (s, 1 H), 10.77 (s, 1 H). ¹⁹F NMR (377 MHz, DMSO-d6) δ ppm −116.84 (s, 1 F). |
| TBM-5 | CBM-3 | 34 | 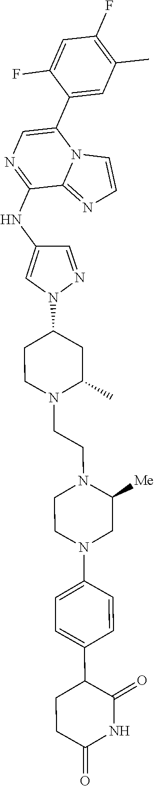 [SMILES: Cc1cc(-c2cnc(Nc3cnn([C@H]4CCN(CCN5CCN(c6ccc(C7CCC(=O)NC7=O)cc6)C[C@@H]5C)[C@@H](C)C4)c3)c3nccn23)c(F)cc1F] | 3-(4-((S)-4-(2- ((2S,4S)-4-(4-((5- (2,4-difluoro-5- methylphenyl) imdazo [1,2-a]pyrazin- 8-yl)amino)-1H- pyrazol-1-yl)-2- methylpiperidin-1- yl)ethyl)-3- methylpiperazin-1- yl)phenyl) piperidine- 2,6-dione | 31 mg, 11% yield (3 steps), white solid [M + H]]⁺ = 737.4 ¹H NMR (400 MHz, DMSO- d6) δ ppm 1.09 (d, J = 5.9 Hz, 3 H), 1.12 (d, J = 6.1 Hz, 3 H), 1.55-1.68 (m, 1 H), 1.81-2.05 (m, 5 H), 2.07- 2.20 (m, 1 H), 2.29 (s, 3 H), 2.32-2.47 (m, 7 H), 2.57- 2.68 (m, 1 H), 2.75-2.96 (m, 4 H), 3.01-3.10 (m, 1 H), 3.41 (br d, J = 9.3 Hz, 2 H), 3.72 (dd, J = 11.0, 4.9 Hz, 1 H), 4.12-4.23 (m, 1 H), 6.88 (d, J = 8.8 Hz, 2 H), 7.04 (d, J = 8.8 Hz, 2 H), 7.40-7.49 (m, 2 H), 7.58- 7.65 (m, 3 H), 7.78-7.82 (m, 1 H), 8.22 (s, 1 H), 9.98 (s, 1 H), 10.76 (s, 1 H). ¹⁹F NMR (377 MHz, DMSO-d6) δ ppm −112.16 (q, J = 7.7 Hz, 1 F), −111.25 (q, J = 9.1 Hz, 1 F). |
| TBM-5 | CBM-4 | 35 | 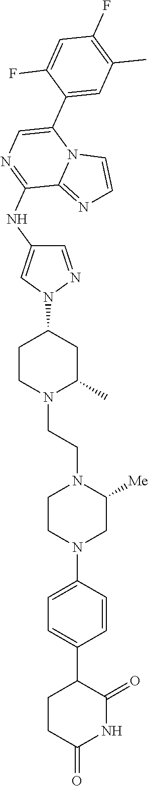 [SMILES: Cc1cc(-c2cnc(Nc3cnn([C@H]4CCN(CCN5CCN(c6ccc(C7CCC(=O)NC7=O)cc6)C[C@H]5C)[C@@H](C)C4)c3)c3nccn23)c(F)cc1F] | 3-(4-((R)-4-(2- ((2S,4S)-4-(4-((5- (2,4-difluoro-5- methylphenyl) imdazo [1,2-a]pyrazin- 8-yl)amino)-1H- pyrazol-1-yl)-2- methylpiperidin-1- yl)ethyl)-3- methylpiperazin-1- yl)phenyl) piperidine- 2,6-dione | 17 mg, 9% yield (3 steps), white solid [M + H]]⁺ = 737.4 ¹H NMR (400 MHz, DMSO- d6) δ ppm 1.09 (d, J = 5.9 Hz, 3 H), 1.12 (d, J = 6.1 Hz, 3 H), 1.61 (q, J = 11.9 Hz, 1 H), 1.79-1.92 (m, 1 H), 1.93-2.05 (m, 3 H), 2.07- 2.18 (m, 1 H), 2.27-2.47 (m, 10 H), 2.53-2.58 (m, 1 H), 2.58-2.65 (m, 1 H), 2.74-2.98 (m, 4 H), 3.02- 3.10 (m, 1 H), 3.41 (br d, J = 11.0 Hz, 2 H), 3.72 (dd, J = 11.0, 4.9 Hz, 1 H), 4.11- 4.24 (m, 1 H), 6.88 (d, J = 8.8 Hz, 2 H), 7.04 (d, J = 8.8 Hz, 2 H), 7.42-7.48 (m, 2 H), 7.58-7.64 (m, 3 H), 7.79 (s, 1 H), 8.22 (s, 1 H), 9.98 (s, 1 H), 10.76 (s, 1 H). ¹⁹F NMR (377 MHz, DMSO-d6) δ ppm −112.22- −112.10 (m, 1 F), −111.32- −111.19 (m, 1 F). |
| TBM-9 | CBM-2 | 36 | 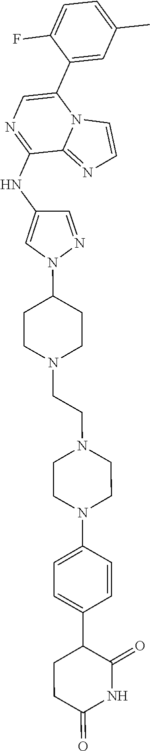 [SMILES: Cc1ccc(F)c(-c2cnc(Nc3cnn(C4CCN(CCN5CCN(c6ccc(C7CCC(=O)NC7=O)cc6)CC5)CC4)c3)c3nccn23)c1] | 3-(4-(4-(2-(4-(4- ((5-(2-fluoro-5- methylphenyl) imdazo [1,2-a]pyrazin- 8-yl)amino)-1H- pyrazol-1- yl)piperidin-1- yl)ethyl)piperazin- 1- yl)phenyl) piperidine- 2,6-dione | 56 mg, 18% yield (3 steps), white solid [M + H]]⁺ = 691.4. ¹H NMR (400 MHz, DMSO- d6 ) δ ppm 1.88-2.04 (m, 5 H), 2.07-2.18 (m, 3 H), 2.37 (s, 3 H), 2.54-2.68 (m, 5 H), 3.01 (br d, J = 11.2 Hz, 2 H), 3.08-3.15 (m, 4 H), 3.72 (dd, J = 10.9, 5.0 Hz, 1 H), 4.09-4.17 (m, 1 H), 6.89 (d, J = 8.6 Hz, 2 H), 7.04 (d, J = 8.6 Hz, 2 H), 7.33 (t, J = 9.8 Hz, 1 H), 7.37-7.47 (m, 3 H), 7.56- 7.59 (m, 1 H), 7.62 (d, J = 1.0 Hz, 1 H), 7.80 (s, 1 H), 8.24 (s, 1 H), 9.97 (s, 1 H), 10.77 (s, 1 H). ¹⁹F NMR (377 MHz, DMSO-d6) δ ppm −117.43- −117.35 (m, 1 F). |
| TBM-7 | CBM-3 | 38 | 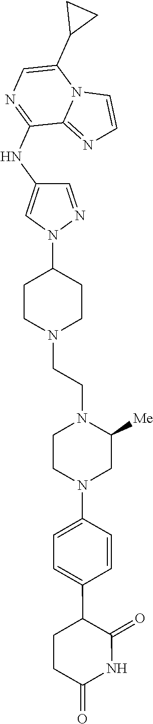 [SMILES: C[C@H]1CN(c2ccc(C3CCC(=O)NC3=O)cc2)CCN1CCN1CCC(n2cc(Nc3ncc(C4CC4)n4ccnc34)cn2)CC1] | 3-(4-((S)-4-(2-(4- (4-((5- cyclopropylimidaz o[1,2-a]pyrazin-8- yl)amino)-1H- pyrazol-1- yl)piperidin-1- yl)ethyl)-3- methylpiperazin-1- yl)phenyl) piperidine- 2,6-dione | 2.5 mg, 3% yield (3 steps), white solid [M + H]]⁺ = 637.4; [M + 2H]2⁺ =319.2. ¹H NMR (400 MHz, DMSO- d6) δ ppm 0.69-0.77 (m, 2 H), 0.97-1.03 (m, 2 H), 1.08 (d, J = 5.9 Hz, 3 H), 1.82-2.19 (m, 10 H), 2.34- 2.46 (m, 4 H), 2.57-2.69 (m, 1 H), 2.74-2.89 (m, 2 H), 2.90-3.06 (m, 3 H), 3.41 (br d, J = 10.3 Hz, 3 H), 3.72 (dd, J = 11.0, 4.9 Hz, 1 H), 4.04-4.14 (m, 1 H), 6.88 (d, J = 8.8 Hz, 2 H), 7.04 (d, J = 8.8 Hz, 2 H), 7.22 (d, J = 1.2 Hz, 1 H), 7.65 (d, J = 1.0 Hz, 1 H), 7.74 (s, 1 H), 8.05 (d, J = 1.2 Hz, 1 H), 8.17 (s, 1 H), 9.64 (s, 1 H), 10.77 (s, 1 H). |
| TBM-2 | CBM-10 | 39 | 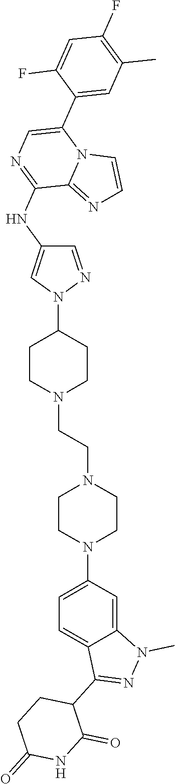 [SMILES: Cc1cc(-c2cnc(Nc3cnn(C4CCN(CCN5CCN(c6ccc7c(C8CCC(=O)NC8=O)nn(C)c7c6)CC5)CC4)c3)c3nccn23)c(F)cc1F] | 3-(6-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1-yl)-1-methyl- 1H-indazol-3- yl)piperidine-2,6- dione | 32 mg, 16% yield (3 steps), white solid [M + H]]⁺ = 763.4 ¹H NMR (400 MHz, DMSO- d6) δ ppm 1.86-2.04 (m, 4 H), 2.09-2.20 (m, 3 H), 2.32 (br s, 4 H), 2.51-2.53 (m, 4 H), 2.56-2.65 (m, 6 H), 3.01 (br d, J = 11.5 Hz, 2 H), 3.18-3.24 (m, 4 H), 3.89 (s, 3 H), 4.08-4.19 (m, 1 H), 4.25 (dd, J = 9.2, 5.0 Hz, 1 H), 6.84 (d, J = 1.7 Hz, 1 H), 6.92 (dd, J = 8.9, 1.8 Hz, 1 H), 7.42-7.51 (m, 3 H), 7.58-7.64 (m, 3 H), 7.80 (s, 1 H), 8.24 (s, 1 H), 9.98 (s, 1 H), 10.84 (s, 1 H). ¹⁹F NMR (377 MHz, DMSO-d6) δ ppm −112.17 (q, J = 8.6 Hz, 1 F), −111.25 (q, J = 8.2 Hz, 1 F). |
| TBM-2 | CBM-11 | 41 | 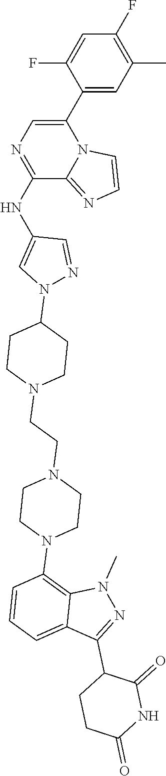 [SMILES: Cc1cc(-c2cnc(Nc3cnn(C4CCN(CCN5CCN(c6cccc7c(C8CCC(=O)NC8=O)nn(C)c67)CC5)CC4)c3)c3nccn23)c(F)cc1F] | 3-(7-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1-yl)-1-methyl- 1H-indazol-3- yl)piperidine-2,6- dione | 53 mg, 35% yield (3 steps), white solid [M + H]]⁺ = 763.5 ¹H NMR (400 MHz, DMSO- d6) δ ppm 1.86-2.03 (m, 4 H), 2.09-2.21 (m, 3 H), 2.26-2.38 (m, 5 H), 2.52- 2.72 (m, 6 H), 2.74-3.22 (m, 7 H), 4.08-4.19 (m, 1 H), 4.25 (s, 3 H), 4.33 (dd, J = 9.5, 5.1 Hz, 1 H), 6.98- 7.07 (m, 2 H), 7.39 (dd, J = 7.2, 1.6 Hz, 1 H), 7.41-7.48 (m, 2 H), 7.57-7.64 (m, 3 H), 7.80 (s, 1 H), 8.24 (s, 1 H), 9.98 (s, 1 H), 10.88 (s, 1 H). ¹⁹F NMR (377 MHz, DMSO-d6) δ ppm −112.17 (q, J = 9.5 Hz, 1 F), −111.31- −111.19 (m, 1 F). |
| TBM-2 | CBM-12 | 42 | 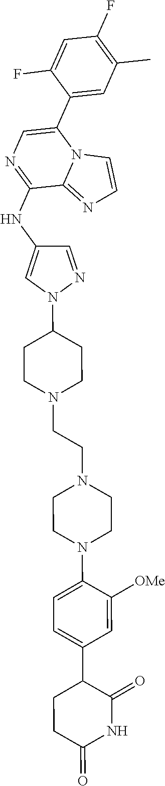 [SMILES: COc1cc(C2CCC(=O)NC2=O)ccc1N1CCN(CCN2CCC(n3cc(Nc4ncc(-c5cc(C)c(F)cc5F)n5ccnc45)cn3)CC2)CC1] | 3-(4-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1-yl)-3- methoxyphenyl) piperidine- 2,6-dione | 17 mg, 7% yield (3 steps), white solid [M + H]]⁺ = 739.3. ¹H NMR (400 MHz, DMSO- d6) δ ppm 1.85-2.25 (m, 9 H), 2.27-2.31 (m, 3 H), 2.43-2.48 (m, 1 H), 2.53- 2.69 (m, 6 H), 2.91-3.04 (m, 6 H), 3.72-3.79 (m, 4 H), 4.08- 4.18 (m, 1 H), 6.70 (dd, J = 8.1, 1.7 Hz, 1 H), 6.78-6.84 (m, 2 H), 7.42-7.48 (m, 2 H), 7.58-7.64 (m, 3 H), 7.80 (s, 1 H), 8.24 (s, 1 H), 9.98 (s, 1 H), 10.78 (s, 1 H). ¹⁹F NMR (377 MHz, DMSO-d6) δ ppm −112.17 (q, J = 8.8 Hz, 1 F), −111.26 (q, J = 8.8 Hz, 1 F). |
| TBM-2 | CBM-13 | 43 | 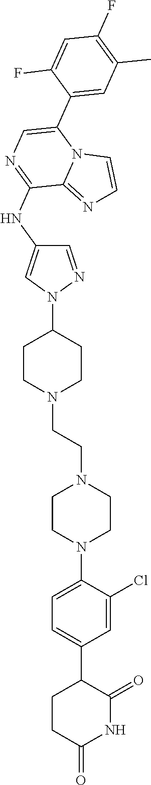 [SMILES: Cc1cc(-c2cnc(Nc3cnn(C4CCN(CCN5CCN(c6ccc(C7CCC(=O)NC7=O)cc6Cl)CC5)CC4)c3)c3nccn23)c(F)cc1F] | 3-(3-chloro-4-(4- (2-(4-(4-((5-(2,4- difluoro-5- methylphenyl) imdazo [1,2-a]pyrazin- 8-yl)amino)-1H- pyrazol-1- yl)piperidin-1- yl)ethyl)piperazin- 1- yl)phenyl) piperidine- 2,6-dione | 27 mg, 18% yield (3 steps), white solid [M + H]]⁺ = 743.3. ¹H NMR (400 MHz, DMSO- d6) δ ppm 1.89-2.05 (m, 5 H), 2.10-2.25 (m, 3 H), 2.29 (s, 3 H), 2.44-2.49 (m, 1 H), 2.52 (br s, 4 H), 2.56- 2.70 (m, 5 H), 2.93-3.05 (m, 6 H), 3.82 (dd, J = 12.1, 4.8 Hz, 1 H), 4.09-4.19 (m, 1 H), 7.10-7.16 (m, 2 H), 7.28 (d, J = 2.0 Hz, 1 H), 7.42-7.48 (m, 2 H), 7.58- 7.63 (m, 3 H), 7.80 (s, 1 H), 8.24 (s, 1 H), 9.98 (s, 1 H), 10.83 (s, 1 H). ¹⁹F NMR (377 MHz, DMSO-d6) δ ppm −112.16 (q, J = 8.8 Hz, 1 F), −111.25 (q, J = 9.0 Hz, 1 F). |
| TBM-2 | CBM-14 | 44 | 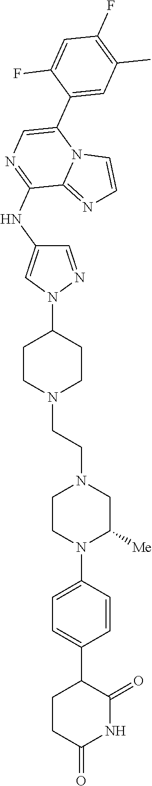 [SMILES: Cc1cc(-c2cnc(Nc3cnn(C4CCN(CCN5CCN(c6ccc(C7CCC(=O)NC7=O)cc6)[C@@H](C)C5)CC4)c3)c3nccn23)c(F)cc1F] | 3-(4-((S)-4-(2-(4- (4-((5-(2,4- difluoro-5- methylphenyl) imdazo [1,2-a]pyrazin- 8-yl)amino)-1H- pyrazol-1- yl)piperidin-1- yl)ethyl)-2- methylpiperazin-1- yl)phenyl) piperidine- 2,6-dione | 62 mg, 20% yield (3 steps), white solid [M + H]]⁺ = 723.4 ¹H NMR (400 MHz, DMSO- d6) δ ppm 1.00 (d, J = 6.4 Hz, 3 H), 1.86-2.04 (m, 5 H), 2.04-2.25 (m, 5 H), 2.29 (s, 3 H), 2.30-2.37 (m, 1 H), 2.42-2.48 (m, 2 H), 2.52-2.56 (m, 2 H), 2.58- 2.68 (m, 1 H), 2.73 (br d, J = 11.2 Hz, 1 H), 2.87-2.99 (m, 2 H), 3.04 (br d, J = 10.5 Hz, 2 H), 3.20-3.27 (m, 1 H), 3.71 (dd, J = 10.8, 4.9 Hz, 1 H), 3.92 (br s, 1 H), 4.09-4.19 (m, 1 H), 6.84 (d, J = 8.6 Hz, 2 H), 7.04 (d, J = 8.6 Hz, 2 H), 7.41-7.48 (m, 2 H), 7.58-7.63 (m, 3 H), 7.80 (s, 1 H), 8.16 (s, 1 H), 8.23 (s, 1 H), 9.98 (s, 1 H), 10.77 (s, 1 H). ¹⁹F NMR (377 MHz, DMSO-d6) δ ppm −112.16 (q, J = 9.1 Hz, 1 F), −111.25 (q, J = 9.1 Hz, 1 F). |
| TBM-7 | CBM-2 | 46 | 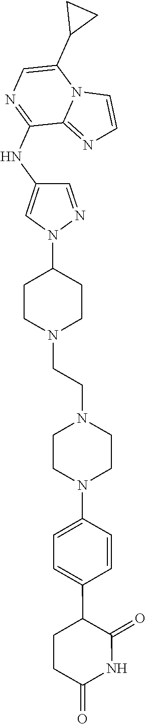 [SMILES: O=C1CCC(c2ccc(N3CCN(CCN4CCC(n5cc(Nc6ncc(C7CC7)n7ccnc67)cn5)CC4)CC3)cc2)C(=O)N1] | 3-(4-(4-(2-(4-(4- ((5- cyclopropylimidaz o[1,2-a]pyrazin-8- yl)amino)-1H- pyrazol-1- yl)piperidin-1- yl)ethyl)piperazin- 1- yl)phenyl) piperidine- 2,6-dione | 14 mg, 14% yield (3 steps), white solid [M + H]]⁺ = 623.4; [M + 2H]²⁺ = 312.2. ¹H NMR (400 MHz, DMSO- d6) δ ppm 0.68-0.77 (m, 2 H), 0.95-1.04 (m, 2 H), 1.83-2.07 (m, 6 H), 2.08- 2.20 (m, 3 H), 2.40-2.48 (m, 2 H), 2.54-2.68 (m, 5 H), 3.00 (br d, J = 11.7 Hz, 2 H), 3.07-3.15 (m, 4 H), 3.72 (dd, J = 10.9, 5.0 Hz, 1 H), 4.05-4.15 (m, 1 H), 6.89 (d, J = 8.6 Hz, 2 H), 7.05 (d, J = 8.6 Hz, 2 H), 7.22 (d, J = 1.2 Hz, 1 H), 7.65 (d, J = 1.0 Hz, 1 H), 7.74 (s, 1 H), 8.05 (d, J = 1.0 Hz, 1 H), 8.17 (s, 1 H), 9.64 (s, 1 H), 10.77 (s, 1 H). |
| TBM-10 | CBM-2 | 48 | 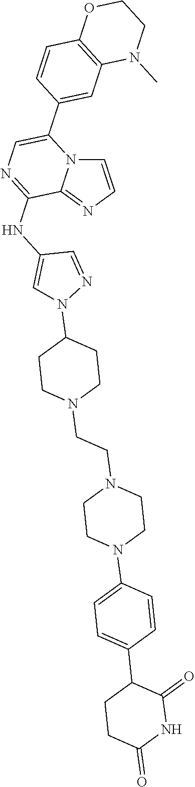 [SMILES: CN1CCOc2ccc(-c3cnc(Nc4cnn(C5CCN(CCN6CCN(c7ccc(C8CCC(=O)NC8=O)cc7)CC6)CC5)c4)c4nccn34)cc21] | 3-(4-(4-(2-(4-(4- ((5-(4-methyl-3,4- dihydro-2H- benzo[b][1,4] oxazin-6- yl)imidazo[1,2- a]pyrazin-8- yl)amino)-1H- pyrazol-1- yl)piperidin-1- yl)ethyl)piperazin- 1- yl)phenyl) piperidine- 2,6-dione | 79 mg, 18% yield (3 steps), white solid [M + H]]⁺ = 730.4 ¹H NMR (400 MHz, DMSO- d6) δ ppm 1.86-2.06 (m, 5 H), 2.07-2.20 (m, 3 H), 2.42-2.48 (m, 2 H), 2.52- 2.68 (m, 6 H), 2.89 (s, 3 H), 3.01 (br d, J = 12.0 Hz, 2 H), 3.07-3.15 (m, 4 H), 3.72 (dd, J = 10.9, 5.0 Hz, 1 H), 4.06-4.17 (m, 1 H), 4.26- 4.33 (m, 2 H), 6.80-6.94 (m, 5 H), 7.05 (d, J = 8.8 Hz, 2 H), 7.37 (s, 1 H), 7.56- 7.65 (m, 1 H), 7.78 (s, 1 H), 7.88 (d, J = 1.2 Hz, 1 H), 8.24 (s, 1 H), 9.79 (s, 1 H), 10.77 (s, 1 H). |
| TBM-2 | CBM-15 | 49 | 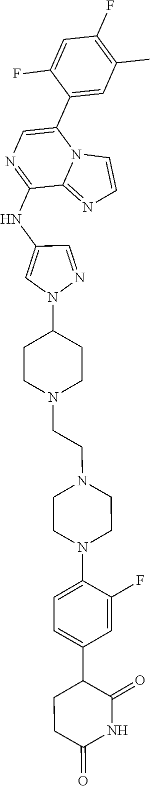 [SMILES: Cc1cc(-c2cnc(Nc3cnn(C4CCN(CCN5CCN(c6ccc(C7CCC(=O)NC7=O)cc6F)CC5)CC4)c3)c3nccn23)c(F)cc1F] | 3-(4-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1-yl)-3- fluorophenyl)piper idine-2,6-dione | 14 mg, 13% yield (3 steps), white solid [M + H]]⁺ = 727.3 ¹H NMR (400 MHz, DMSO- d6) δ ppm 1.86-2.05 (m, 5 H), 2.09-2.25 (m, 3 H), 2.29 (s, 3 H), 2.52-2.71 (m, 6 H), 3.00 (br s, 6 H), 3.80 (dd, J = 11.7, 4.9 Hz, 1 H), 4.06-4.21 (m, 1 H), 6.92- 7.08 (m, 3 H), 7.40-7.50 (m, 2 H), 7.57-7.67 (m, 3 H), 7.78-7.84 (m, 1 H), 8.23 (s, 1 H), 9.98 (s, 1 H), 10.81 (s, 1 H). ¹⁹F NMR (377 MHz, DMSO-d6) δ ppm −122.93 (dd, J = 13.6, 8.2 Hz, 1 F), −112.45-−111.96 (m, 1 F), −111.53-−110.93 (m, 1 F). |
| TBM-2 | CBM-16 | 50 | 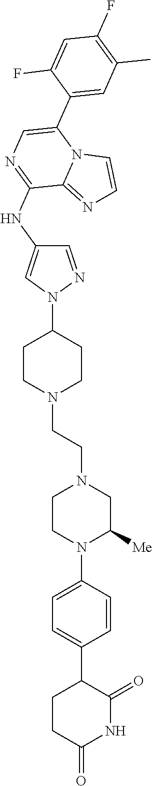 [SMILES: Cc1cc(-c2cnc(Nc3cnn(C4CCN(CCN5CCN(c6ccc(C7CCC(=O)NC7=O)cc6)[C@H](C)C5)CC4)c3)c3nccn23)c(F)cc1F] | 3-(4-((R)-4-(2-(4- (4-((5-(2,4- difluoro-5- methylphenyl) imdazo [1,2-a]pyrazin- 8-yl)amino)-1H- pyrazol-1- yl)piperidin-1- yl)ethyl)-2- methylpiperazin-1- yl)phenyl) piperidine- 2,6-dione | 14 mg, 10% yield (3 steps), white solid [M + H]]⁺ = 723.3 ¹H NMR (400 MHz, DMSO- d6) δ ppm 1.00 (d, J = 6.4 Hz, 3 H), 1.86-2.06 (m, 6 H), 2.06-2.21 (m, 4 H), 2.27-2.37 (m, 5 H), 2.41- 2.48 (m, 3 H), 2.58-2.69 (m, 1 H), 2.73 (br d, J = 9.5 Hz, 1 H), 2.86-2.98 (m, 2 H), 2.98-3.07 (m, 2 H), 3.20-3.27 (m, 1 H), 3.72 (dd, J = 10.8, 5.1 Hz, 1 H), 3.88-4.00 (m, 1 H), 4.08- 4.18 (m, 1 H), 6.84 (d, J = 8.6 Hz, 2 H), 7.04 (d, J = 8.8 Hz, 2 H), 7.40-7.50 (m, 2 H), 7.58-7.65 (m, 3 H), 7.78-7.83 (m, 1 H), 8.23 (s, 1 H), 9.98 (s, 1 H), 10.76 (s, 1 H). ¹⁹F NMR (377 MHz, DMSO-d6) δ ppm −112.17 (q, J = 8.6 Hz, 1 F), −111.33- −111.19 (m, 1 F). |
| TBM-11 | CBM-2 | 51 | 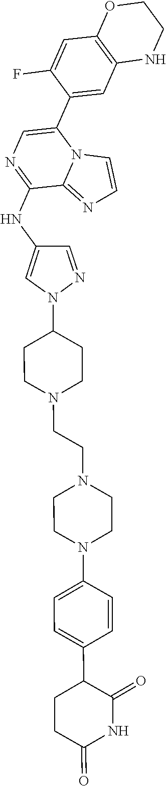 [SMILES: O=C1CCC(c2ccc(N3CCN(CCN4CCC(n5cc(Nc6ncc(-c7cc8c(cc7F)OCCN8)n7ccnc67)cn5)CC4)CC3)cc2)C(=O)N1] | 3-(4-(4-(2-(4-(4- ((5-(7-fluoro-3,4- dihydro-2H- benzo[b][1,4] oxazin-6- yl)imidazo[1,2- a]pyrazin-8- yl)amino)-1H- pyrazol-1- yl)piperidin-1- yl)ethyl)piperazin- 1- yl)phenyl) piperidine- 2,6-dione | 71 mg, 25% yield (3 steps), white solid [M + H]]⁺ = 734.4 ¹H NMR (400 MHz, DMSO- d6) δ ppm 1.87-2.05 (m, 5 H), 2.07-2.20 (m, 3 H), 2.40-2.48 (m, 2 H), 2.53- 2.70 (m, 6 H), 3.00 (br d, J = 12.0 Hz, 2 H), 3.11 (br s, 4 H), 3.72 (dd, J = 11.0, 4.6 Hz, 1 H), 4.07-4.18 (m, 1 H), 4.22 (br s, 2 H), 5.90 (s, 1 H), 6.71 (d, J = 7.3 Hz, 1 H), 6.77 (d, J = 11.0 Hz, 1 H), 6.89 (d, J = 8.8 Hz, 2 H), 7.04 (d, J = 8.6 Hz, 2 H), 7.31-7.40 (m, 1 H), 7.55 (d, J = 1.7 Hz, 1 H), 7.60 (d, J = 1.0 Hz, 1 H), 7.78 (s, 1 H), 8.23 (s, 1 H), 9.88 (s, 1 H), 10.77 (s, 1 H). ¹⁹F NMR (377 MHz, DMSO-d6) δ ppm −125.93- −125.84 (m, 0.85 F), −125.84- −125.78 (m, 0.15 F). |
| TBM-60 | CBM-2 | 52 | 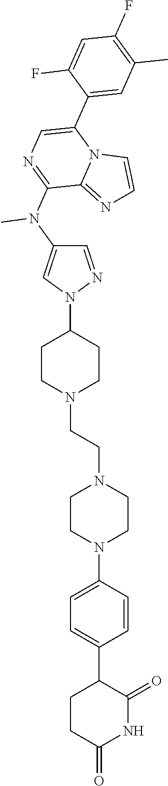 | 3-(4-(4-(2-(4-(4- ((5-(2,4-difluoro- 5- methylphenyl) imdazo [1,2-a]pyrazin- 8- yl)(methyl)amino)- 1H-pyrazol-1- yl)piperidin-1- yl)ethyl)piperazin- 1- yl)phenyl) piperidine- 2,6-dione | *skipped first step 31 mg, 35% yield (2 steps), white solid [M + H]]⁺ = 723.4 ¹H NMR (400 MHz, DMSO- d6) δ ppm 1.88-2.05 (m, 6 H), 2.07-2.18 (m, 4 H), 2.28 (s, 4 H), 2.42-2.45 (m, 2 H), 2.54-2.64 (m, 5 H), 2.97-3.04 (m, 2 H), 3.11 (br s, 4 H), 3.69-3.76 (m, 1 H), 3.97 (s, 3 H), 4.06-4.16 (m, 1 H), 6.89 (d, J = 8.6 Hz, 2 H), 7.04 (d, J = 8.6 Hz, 2 H), 7.41-7.49 (m, 2 H), 7.55- 7.62 (m, 3 H), 7.65-7.68 (m, 1 H), 8.08 (s, 1 H), 10.76 (s, 1 H). ¹⁹F NMR (377 MHz, DMSO-d6) δ ppm −111.96 (q, J = 7.7 Hz, 1 F), −111.19- −110.93 (m, 1 F). |
| TBM-2 | CBM-17 | 53 | 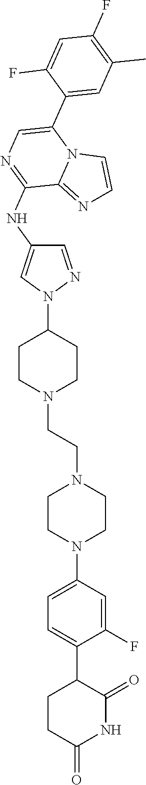 [SMILES: Cc1cc(-c2cnc(Nc3cnn(C4CCN(CCN5CCN(c6ccc(C7CCC(=O)NC7=O)c(F)c6)CC5)CC4)c3)c3nccn23)c(F)cc1F] | 3-(4-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1-yl)-2- fluorophenyl)piper idine-2,6-dione | 40 mg, 19% yield (3 steps), white solid [M + H]]⁺ = 727.3; [M + 2H]²⁺ = 364.2 ¹H NMR (400 MHz, DMSO-d6) δ ppm 1.93- 2.08 (m, 5 H), 2.12-2.29 (m, 3 H), 2.33 (s, 3 H), 2.55- 2.58 (m, 3 H), 2.58-2.64 (m, 6 H), 2.69-2.81 (m, 1 H), 3.08 (br d, J = 11.0 Hz, 2 H), 3.16-3.23 (m, 4 H), 3.92 (dd, J = 12.3, 5.0 Hz, 1 H), 4.14-4.24 (m, 1 H), 6.74-6.79 (m, 2 H), 7.12 (t, J = 8.9 Hz, 1 H), 7.46-7.52 (m, 2 H), 7.62-7.68 (m, 3 H), 7.84 (s, 1 H), 8.27 (s, 1 H), 10.02 (s, 1 H), 10.84 (s, 1 H) ¹⁹F NMR (377 MHz, DMSO-d6) δ ppm −115.96- −115.76 (m, 1 F), −112.17 (s, 1 F), −111.24 (s, 1 F). |
| TBM-2 | CBM-18 | 54 | 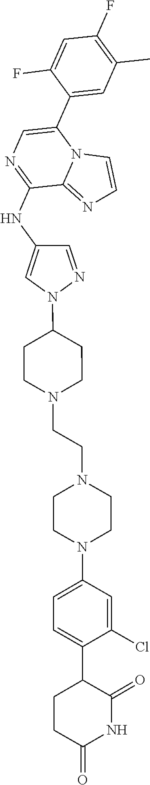 [SMILES: Cc1cc(-c2cnc(Nc3cnn(C4CCN(CCN5CCN(c6ccc(C7CCC(=O)NC7=O)c(Cl)c6)CC5)CC4)c3)c3nccn23)c(F)cc1F] | 3-(2-chloro-4-(4- (2-(4-(4-((5-(2,4- difluoro-5- methylphenyl) imdazo [1,2-a]pyrazin- 8-yl)amino)-1H- pyrazol-1- yl)piperidin-1- yl)ethyl)piperazin- 1- yl)phenyl) piperidine- 2,6-dione | 19 mg, 8% yield (3 steps), white solid [M + H]]⁺ = 743.3; [M + 2H]²⁺ = 372.2 ¹H NMR (400 MHz, DMSO- d6) δ ppm 1.85-2.03 (m, 5 H), 2.12 (t, J = 11.4 Hz, 2 H), 2.17-2.35 (m, 1 H), 2.29 (s, 3 H), 2.53-2.60 (m, 5 H), 2.65-2.82 (m, 2 H), 3.00 (br d, J = 11.2 Hz, 2 H), 3.15 (br s, 4 H), 4.05 (dd, J = 12.2, 4.9 Hz, 1 H), 4.08- 4.18 (m, 1 H), 6.89 (d, J = 8.8 Hz, 1 H), 6.94 (s, 1 H), 7.11 (d, J = 8.6 Hz, 1 H), 7.41-7.49 (m, 2 H), 7.57- 7.66 (m, 3 H), 7.80 (s, 1 H), 8.23 (s, 1 H), 9.98 (s, 1 H), 10.83 (s, 1 H). ¹⁹F NMR (377 MHz, DMSO-d6) δ ppm −112.16 (s, 1 F), −111.25 (s, 1 F). |
| TBM-2 | CBM-19 | 55 | 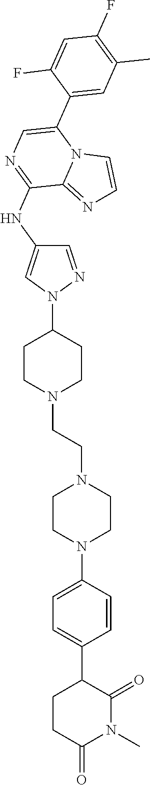 [SMILES: Cc1cc(-c2cnc(Nc3cnn(C4CCN(CCN5CCN(c6ccc(C7CCC(=O)N(C)C7=O)cc6)CC5)CC4)c3)c3nccn23)c(F)cc1F] | 3-(4-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1-yl)phenyl)-1- methylpiperidine- 2,6-dione | 38 mg, 23% yield (3 steps), white solid [M + H]]⁺ =723.4; [M + 2H]²⁺ = 362.2 ¹H NMR (400 MHz, DMSO- d6) δ ppm 1.83-2.05 (m, 5 H), 2.06-2.21 (m, 3 H), 2.29 (s, 3 H), 2.53-2.59 (m, 4 H), 2.60-2.65 (m, 1 H), 2.69-2.80 (m, 1 H), 2.97- 3.05 (m, 5 H), 3.06-3.15 (m, 4 H), 3.83 (dd, J = 11.0, 4.9 Hz, 1 H), 4.08-4.19 (m, 1 H), 6.88 (d, J = 8.8 Hz, 2 H), 7.04 (d, J = 8.6 Hz, 2 H), 7.41-7.49 (m, 2 H), 7.57- 7.65 (m, 3 H), 7.80 (s, 1 H), 8.23 (s, 1 H), 9.98 (s, 1 H). ¹⁹F NMR (377 MHz, DMSO-d6) δ ppm −112.16 (s, 1 F), −111.25 (s, 1 F). |
| TBM-2 | CBM-20 | 56 | 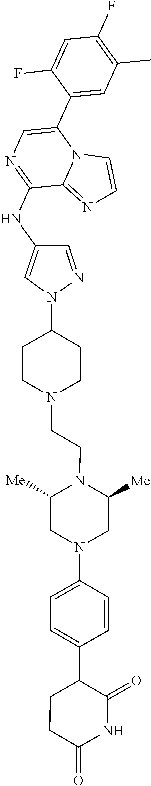 [SMILES: Cc1cc(-c2cnc(Nc3cnn(C4CCN(CCN5[C@@H](C)CN(c6ccc(C7CCC(=O)NC7=O)cc6)C[C@@H]5C)CC4)c3)c3nccn23)c(F)cc1F] | 3-(4-((3S,5S)-4-(2- (4-(4-((5-(2,4- difluoro-5- methylphenyl) imdazo [1,2-a]pyrazin- 8-yl)amino)-1H- pyrazol-1- yl)piperidin-1- yl)ethyl)-3,5- dimethylpiperazin- 1- yl)phenyl) piperidine- 2,6-dione | 51 mg, 25% yield (3 steps), white solid [M + H]]⁺ = 737.3, [M + 2H]²⁺ = 369.3. ¹H NMR (400 MHz, DMSO- d6) δ ppm 1.07 (s, 3 H), 1.08 (s, 3 H), 1.90-2.06 (m, 5 H), 2.06-2.18 (m, 1 H), 2.18-2.31 (m, 5 H), 2.41- 2.49 (m, 2 H), 2.52-2.69 (m, 3 H), 2.86 (br dd, J = 11.5, 6.4 Hz, 3 H), 2.99- 3.11 (m, 4 H), 3.14 (br dd, J = 11.6, 1.6 Hz, 2 H), 3.72 (dd, J = 11.0, 4.9 Hz, 1 H), 4.11-4.22 (m, 1 H), 6.87 (d, J = 8.8 Hz, 2 H), 7.04 (d, J = 8.8 Hz, 2 H), 7.42-7.48 (m, 2 H), 7.58-7.64 (m, 3 H), 7.81 (s, 1 H), 8.24 (s, 1 H), 9.99 (s, 1 H), 10.76 (s, 1 H). ¹⁹F NMR (377 MHz, DMSO-d6) δ ppm −112.18 (q, J = 8.2 Hz, 1 F), −111.24 (q, J = 7.7 Hz, 1 F). |
| TBM-2 | CBM-21 | 58 | 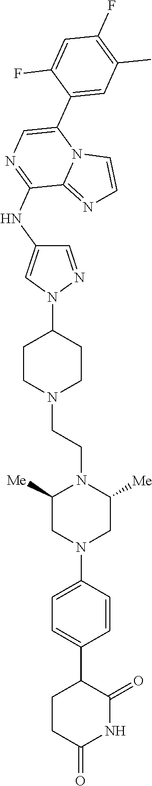 [SMILES: Cc1cc(-c2cnc(Nc3cnn(C4CCN(CCN5[C@H](C)CN(c6ccc(C7CCC(=O)NC7=O)cc6)C[C@H]5C)CC4)c3)c3nccn23)c(F)cc1F] | 3-(4-((3R,5R)-4- (2-(4-(4-((5-(2,4- difluoro-5- methylphenyl) imdazo [1,2-a]pyrazin- 8-yl)amino)-1H- pyrazol-1- yl)piperidin-1- yl)ethyl)-3,5- dimethylpiperazin- 1- yl)phenyl) piperidine- 2,6-dione | 71 mg, 35% yield (3 steps), light-yellow solid [M + H]]⁺ = 737.4 ¹H NMR (400 MHz, DMSO- d6) δ ppm 1.07 (d, J = 6.4 Hz, 6 H), 1.89-2.05 (m, 5 H), 2.07-2.26 (m, 3 H), 2.29 (s, 3 H), 2.41-2.48 (m, 3 H), 2.57-2.68 (m, 1 H), 2.77-2.92 (m, 3 H), 3.03 (br d, J = 2.7 Hz, 4 H), 3.10- 3.18 (m, 2 H), 3.72 (dd, J = 11.0, 5.1 Hz, 1 H), 4.08- 4.23 (m, 1 H), 6.87 (d, J = 8.8 Hz, 2 H), 7.03 (d, J = 8.6 Hz, 2 H), 7.41-7.49 (m, 2 H), 7.57-7.65 (m, 3 H), 7.79-7.83 (m, 1 H), 8.24 (s, 1 H), 9.98 (s, 1 H), 10.76 (s, 1 H). ¹⁹F NMR (377 MHz, DMSO-d6) δ ppm −112.17 (q, J = 8.2 Hz, 1 F), −111.24 (q, J = 7.7 Hz, 1 F). |
| TBM-6 | CBM-3 | 59 | 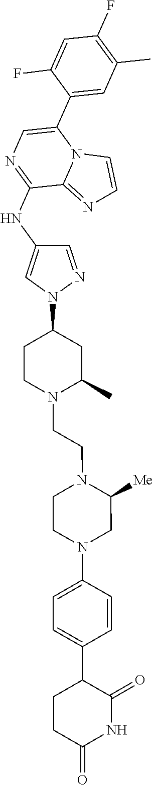 [SMILES: Cc1cc(-c2cnc(Nc3cnn([C@@H]4CCN(CCN5CCN(c6ccc(C7CCC(=O)NC7=O)cc6)C[C@@H]5C)[C@H](C)C4)c3)c3nccn23)c(F)cc1F] | 3-(4-((S)-4-(2- ((2R,4R)-4-(4-((5- (2,4-difluoro-5- methylphenyl) imdazo [1,2-a]pyrazin- 8-yl)amino)-1H- pyrazol-1-yl)-2- methylpiperidin-1- yl)ethyl)-3- methylpiperazin-1- yl)phenyl) piperidine- 2,6-dione | 9 mg, 5% yield (3 steps), white solid [M + H]]⁺ = 737.4; [M + 2H]²⁺ = 369.3 ¹H NMR (400 MHz, DMSO- d6) δ ppm 1.09 (d, J = 5.9 Hz, 3 H), 1.12 (d, J = 5.9 Hz, 3 H), 1.61 (q, J = 11.8 Hz, 1 H), 1.80-1.93 (m, 1 H), 1.94-2.04 (m, 3 H), 2.06- 2.19 (m, 1 H), 2.28-2.30 (m, 3 H), 2.32-2.45 (m, 6 H), 2.53-2.69 (m, 2 H), 2.78 (br t, J = 9.9 Hz, 1 H), 2.83-2.97 (m, 3 H), 3.04- 3.10 (m, 1 H), 3.41 (br d, J = 10.5 Hz, 3 H), 3.72 (dd, J = 11.0, 4.9 Hz, 1 H), 4.13- 4.22 (m, 1 H), 6.88 (d, J = 8.3 Hz, 2 H), 7.04 (d, J = 8.3 Hz, 2 H), 7.42-7.48 (m, 2 H), 7.58-7.64 (m, 3 H), 7.79 (s, 1 H), 8.22 (s, 1 H), 9.98 (s, 1 H), 10.77 (s, 1 H). ¹⁹F NMR (377 MHz, DMSO-d6) δ ppm −112.16 (s, 1 F), −111.26 (s, 1 F) |
| TBM-12 | CBM-2 | 60 | 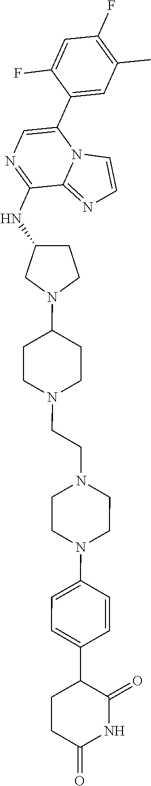 [SMILES: Cc1cc(-c2cnc(N[C@@H]3CCN(C4CCN(CCN5CCN(c6ccc(C7CCC(=O)NC7=O)cc6)CC5)CC4)C3)c3nccn23)c(F)cc1F] | 3-(4-(4-(2-(4-((R)- 3-((5-(2,4- difluoro-5- methylphenyl) imdazo [1,2-a]pyrazin- 8- yl)amino)pyrrolidi n-1-yl)piperidin-1- yl)ethyl)piperazin- 1- yl)phenyl) piperidine- 2,6-dione | 26 mg, 10% yield (3 steps), white solid [M + H]]⁺ = 712.4. ¹H NMR (400 MHz, DMSO- d6) δ ppm 1.33-1.48 (m, 2 H), 1.76-1.85 (m, 2 H), 1.86-1.95 (m, 1 H), 1.97- 2.05 (m, 3 H), 2.06-2.24 (m, 3 H), 2.26-2.29 (m, 3 H), 2.42-2.47 (m, 5 H), 2.52-2.56 (m, 4 H), 2.56- 2.66 (m, 3 H), 2.71-2.79 (m, 1 H), 2.84-2.92 (m, 2 H), 2.92-2.99 (m, 1 H), 3.07-3.12 (m, 4 H), 3.72 (dd, J = 11.0, 4.9 Hz, 1 H), 4.55-4.65 (m, 1 H), 6.88 (d, J = 8.8 Hz, 2 H), 7.04 (d, J = 8.8 Hz, 2 H), 7.31 (s, 1 H), 7.40-7.46 (m, 2 H), 7.52- 7.58 (m, 3 H), 8.19 (s, 1 H), 10.76 (s, 1 H). ¹⁹F NMR (377 MHz, DMSO-d6) δ ppm −112.17 (q, J = 8.2 Hz, 1 F), −111.45 (q, J = 8.2 Hz, 1 F). |
| TBM-13 | CBM-2 | 61 | 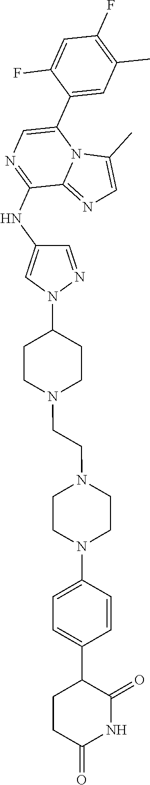 [SMILES: Cc1cc(-c2cnc(Nc3cnn(C4CCN(CCN5CCN(c6ccc(C7CCC(=O)NC7=O)cc6)CC5)CC4)c3)c3ncc(C)n23)c(F)cc1F] | 3-(4-(4-(2-(4-(4- ((5-(2,4-difluoro- 5-methylphenyl)- 3- methylimidazo[1,2- a]pyrazin-8- yl)amino)-1H- pyrazol-1- yl)piperidin-1- yl)ethyl)piperazin- 1- yl)phenyl) piperidine- 2,6-dione | 18 mg, 3% yield (3 steps), white solid [M + H]]⁺ = 723.4. ¹H NMR (400 MHz, DMSO- d6) δ ppm 1.85-1.93 (m, 5 H), 1.93-2.20 (m, 8 H), 2.28 (s, 3 H), 2.41-2.48 (m, 2 H), 2.53-2.66 (m, 6 H), 2.96-3.04 (m, 2 H), 3.08- 3.13 (m, 4 H), 3.72 (dd, J = 11.0, 4.9 Hz, 1 H), 4.07- 4.17 (m, 1 H), 6.88 (d, J = 8.8 Hz, 2 H), 7.04 (d, J = 8.8 Hz, 2 H), 7.24 (s, 1 H), 7.36- 7.42 (m, 2 H), 7.56 (t, J = 8.6 Hz, 1 H), 7.79 (s, 1 H), 8.21 (s, 1 H), 9.83 (s, 1 H), 10.77 (s, 1 H). ¹⁹F NMR (377 MHz, DMSO-d6) δ ppm −113.02 (q, J = 9.1 Hz, 1 F), −110.82 (q, J = 8.6 Hz, 1 F). |
| TBM-14 | CBM-2 | 62 | 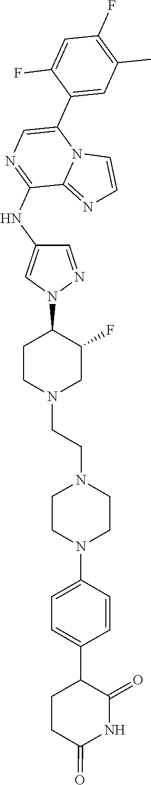 [SMILES: Cc1cc(-c2cnc(Nc3cnn([C@@H]4CCN(CCN5CCN(c6ccc(C7CCC(=O)NC7=O)cc6)CC5)C[C@H]4F)c3)c3nccn23)c(F)cc1F] | 3-(4-(4-(2- ((3R,4R)-4-(4-((5- (2,4-difluoro-5- methylphenyl) imdazo [1,2-a]pyrazin- 8-yl)amino)-1H- pyrazol-1-yl)-3- fluoropiperidin-1- yl)ethyl)piperazin- 1- yl)phenyl) piperidine- 2,6-dione | 29 mg, 6% yield (3 steps), white solid [M + H]]⁺ = 727.4. ¹H NMR (400 MHz, DMSO- d6) δ ppm 1.91-2.05 (m, 3 H), 2.05-2.25 (m, 4 H), 2.29 (s, 3 H), 2.40-2.48 (m, 2 H), 2.54-2.65 (m, 7 H), 2.96 (br d, J = 9.0 Hz, 1 H), 3.12 (t, J = 4.6 Hz, 4 H), 3.34-3.42 (m, 1 H), 3.72 (dd, J = 11.0, 4.9 Hz, 1 H), 4.27-4.40 (m, 1 H), 4.72- 4.93 (m, 1 H), 6.89 (d, J = 8.8 Hz, 2 H), 7.05 (d, J = 8.8 Hz, 2 H), 7.42-7.48 (m, 2 H), 7.58-7.66 (m, 3 H), 7.86 (s, 1 H), 8.31 (s, 1 H), 10.02 (s, 1 H), 10.77 (s, 1 H). ¹⁹F NMR (377 MHz, DMSO-d6) δ ppm −185.58- −185.33 (m, 1 F), −112.18 (q, J = 9.5 Hz, 1 F), −111.23 (q, J = 8.2 Hz, 1 F). |
| TBM-15 | CBM-2 | 63 | 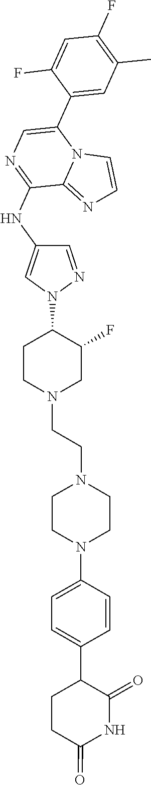 [SMILES: Cc1cc(-c2cnc(Nc3cnn([C@H]4CCN(CCN5CCN(c6ccc(C7CCC(=O)NC7=O)cc6)CC5)C[C@H]4F)c3)c3nccn23)c(F)cc1F] | 3-(4-(4-(2- ((3R,4S)-4-(4-((5- (2,4-difluoro-5- methylphenyl) imdazo [1,2-a]pyrazin- 8-yl)amino)-1H- pyrazol-1-yl)-3- fluoropiperidin-1- yl)ethyl)piperazin- 1- yl)phenyl) piperidine- 2,6-dione | 120 mg, 33% yield (3 steps), white solid [M + H]]⁺ = 727.4. ¹H NMR (400 MHz, DMSO- d6) δ ppm 1.95-2.20 (m, 3 H), 2.24-2.30 (m, 4 H), 2.31-2.46 (m, 2 H), 2.52- 2.69 (m, 8 H), 3.03-3.25 (m, 8 H), 3.72 (dd, J = 10.9, 4.9 Hz, 1 H), 4.32-4.47 (m, 1 H), 4.85-5.07 (m, 1 H), 6.89 (d, J = 8.8 Hz, 2 H), 7.04 (d, J = 8.8 Hz, 2 H), 7.41-7.49 (m, 2 H), 7.58- 7.68 (m, 3 H), 7.85 (s, 1 H), 8.27 (s, 1 H), 10.02 (s, 1 H), 10.77 (s, 1 H). ¹⁹F NMR (377 MHz, DMSO-d6) δ ppm −197.91- −197.31 (m, 1 F), −112.14 (q, J = 8.2 Hz, 1 F), −111.24 (q, J = 9.5 Hz, 1 F). |
| TBM-16 | CBM-2 | 65 | 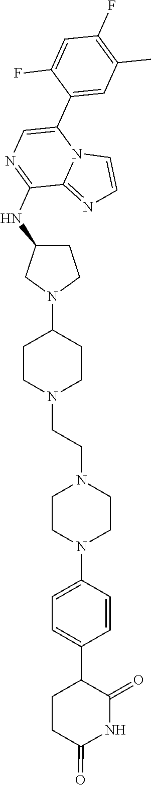 [SMILES: Cc1cc(-c2cnc(N[C@H]3CCN(C4CCN(CCN5CCN(c6ccc(C7CCC(=O)NC7=O)cc6)CC5)CC4)C3)c3nccn23)c(F)cc1F] | 3-(4-(4-(2-(4-((S)- 3-((5-(2,4- difluoro-5- methylphenyl) imdazo [1,2-a]pyrazin- 8- yl)amino)pyrrolidi n-1-yl)piperidin-1- yl)ethyl)piperazin- 1- yl)phenyl) piperidine- 2,6-dione | 89 mg, 23% yield (3 steps), white solid [M + H]]⁺ = 712.4. ¹H NMR (400 MHz, DMSO- d6) δ ppm 1.33-1.46 (m, 2 H), 1.76-1.85 (m, 2 H), 1.86-2.24 (m, 8 H), 2.27 (s, 3 H), 2.43-2.48 (m, 4 H), 2.52-2.56 (m, 4 H), 2.57- 2.67 (m, 3 H), 2.71-2.78 (m, 1 H), 2.85-2.92 (m, 2 H), 2.95 (br t, J = 7.8 Hz, 1 H), 3.05-3.12 (m, 4 H), 3.72 (br dd, J = 10.9, 4.8 Hz, 1 H), 4.55-4.65 (m, 1 H), 6.88 (br d, J = 8.6 Hz, 2 H), 7.04 (d, J = 8.8 Hz, 2 H), 7.31 (s, 1 H), 7.39-7.46 (m, 2 H), 7.51-7.59 (m, 3 H), 10.76 (s, 1 H). ¹⁹F NMR (377 MHz, DMSO-d6) δ ppm −112.17 (d, J = 8.3 Hz, 1 F), −111.45 (d, J = 8.3 Hz, 1 F). |
| TBM-17 | CBM-2 | 68 | 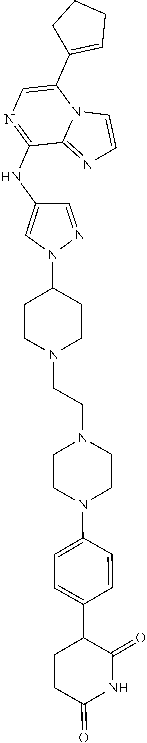 [SMILES: O=C1CCC(c2ccc(N3CCN(CCN4CCC(n5cc(Nc6ncc(C7=CCCC7)n7ccnc67)cn5)CC4)CC3)cc2)C(=O)N1] | 3-(4-(4-(2-(4-(4- ((5-(cyclopent-1- en-1- yl)imidazo[1,2- a]pyrazin-8- yl)amino)-1H- pyrazol-1- yl)piperidin-1- yl)ethyl)piperazin- 1- yl)phenyl) piperidine- 2,6-dione | 14 mg, 6% yield (3 steps), white solid [M + H]]⁺ = 649.4. ¹H NMR (400 MHz, DMSO- d6 ) δ ppm 1.88-2.04 (m, 7 H), 2.08-2.19 (m, 3 H), 2.55-2.69 (m, 7 H), 2.79- 2.85 (m, 2 H), 2.98-3.05 (m, 2 H), 3.09-3.15 (m, 4 H), 3.72 (br dd, J = 10.9, 4.8 Hz, 1 H), 4.08-4.16 (m, 1 H), 6.48 (br s, 1 H), 6.89 (br d, J = 8.6 Hz, 2 H), 7.05 (br d, J = 8.3 Hz, 2 H), 7.48 (s, 1 H), 7.69 (s, 1 H), 7.78 (s, 1 H), 8.15 (s, 1 H), 8.22 (s, 1 H), 8.28 (s, 1 H), 9.90 (s, 1 H), 10.77 (s, 1 H). |
| TBM-18 | CBM-2 | 69 | 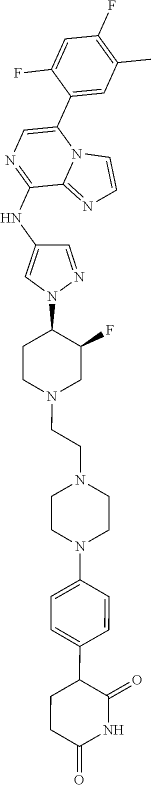 [SMILES: Cc1cc(-c2cnc(Nc3cnn([C@@H]4CCN(CCN5CCN(c6ccc(C7CCC(=O)NC7=O)cc6)CC5)C[C@@H]4F)c3)c3nccn23)c(F)cc1F] | 3-(4-(4-(2- ((3S,4R)-4-(4-((5- (2,4-difluoro-5- methylphenyl) imdazo [1,2-a]pyrazin- 8-yl)amino)-1H- pyrazol-1-yl)-3- fluoropiperidin-1- yl)ethyl)piperazin- 1- yl)phenyl) piperidine- 2,6-dione | 47 mg, 23% yield (3 steps), white solid[ [M + H]]⁺ = 727.3. ¹H NMR (400 MHz, DMSO- d6) δ ppm 1.93-2.06 (m, 2 H), 2.06-2.19 (m, 1 H), 2.23-2.28 (m, 1 H), 2.29 (s, 3 H), 2.34-2.48 (m, 3 H), 2.52-2.65 (m, 9 H), 3.05- 3.17 (m, 5 H), 3.18-3.29 (m, 1 H), 3.72 (dd, J = 10.9, 4.8 Hz, 1 H), 4.31-4.47 (m, 1 H), 4.87-5.06 (m, 1 H), 6.89 (d, J = 8.7 Hz, 2 H), 7.04 (d, J = 8.7 Hz, 2 H), 7.41-7.51 (m, 2 H), 7.58- 7.65 (m, 3 H), 7.85 (s, 1 H), 8.27 (s, 1 H), 10.02 (s, 1 H), 10.77 (s, 1 H). ¹⁹F NMR (377 MHz, DMSO-d6) δ ppm −112.14 (q, J = 8.2 Hz, 1 F), −111.24 (q, J = 7.7 Hz, 1 F). One aliphatic F not observed. |
| TBM-19 | CBM-2 | 72 | 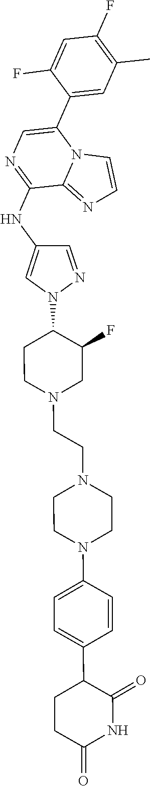 [SMILES: Cc1cc(-c2cnc(Nc3cnn([C@H]4CCN(CCN5CCN(c6ccc(C7CCC(=O)NC7=O)cc6)CC5)C[C@@H]4F)c3)c3nccn23)c(F)cc1F] | 3-(4-(4-(2- ((3S,4S)-4-(4-((5- (2,4-difluoro-5- methylphenyl) imdazo [1,2-a]pyrazin- 8-yl)amino)-1H- pyrazol-1-yl)-3- fluoropiperidin-1- yl)ethyl)piperazin- 1- yl)phenyl) piperidine- 2,6-dione | 60 mg, 33% yield (3 steps), white solid [M + H]]⁺ = 727.4. ¹H NMR (400 MHz, DMSO- d6) δ ppm 1.96-2.06 (m, 3 H), 2.06-2.24 (m, 3 H), 2.29 (s, 3 H), 2.41-2.47 (m, 2 H), 2.55-2.64 (m, 8 H), 2.90-3.00 (m, 1 H), 3.09- 3.13 (m, 4 H), 3.35-3.40 (m, 1 H), 3.72 (dd, J = 10.9, 4.8 Hz, 1 H), 4.27-4.42 (m, 1 H), 4.71-4.96 (m, 1 H), 6.89 (d, J = 8.8 Hz, 2 H), 7.05 (d, J = 8.7 Hz, 2 H), 7.40-7.50 (m, 2 H), 7.57- 7.66 (m, 3 H), 7.86 (s, 1 H), 8.31 (s, 1 H), 10.02 (s, 1 H), 10.77 (s, 1 H). ¹⁹F NMR (377 MHz, DMSO-d6) δ ppm −185.36 (br s, 1 F), −112.18 (q, J = 10.0 Hz, 1 F), −111.23 (q, J = 9.1 Hz, 1 F). |
| TBM-20 | CBM-2 | 73 | 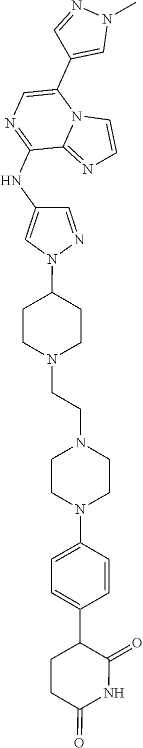 [SMILES: Cn1cc(-c2cnc(Nc3cnn(C4CCN(CCN5CCN(c6ccc(C7CCC(=O)NC7=O)cc6)CC5)CC4)c3)c3nccn23)cn1] | 3-(4-(4-(2-(4-(4- ((5-(1-methyl-1H- pyrazol-4- yl)imidazo[1,2- a]pyrazin-8- yl)amino)-1H- pyrazol-1- yl)piperidin-1- yl)ethyl)piperazin- 1- yl)phenyl) piperidine- 2,6-dione | 10 mg, 4% yield (3 steps), white solid [M + H]]⁺ = 663.3; [M + 2H]²⁺ = 332.2. ¹H NMR (400 MHz, DMSO- d6) δ ppm 1.81-2.05 (m, 5 H), 2.07-2.20 (m, 3 H), 2.41-2.47 (m, 1 H), 2.53- 2.69 (m, 6 H), 3.00 (br d, J = 10.9 Hz, 2 H), 3.10 (d, 4 H), 3.72 (dd, J = 10.8, 4.8 Hz, 1 H), 3.94 (s, 3 H), 4.06-4.17 (m, 1 H), 6.89 (d, J = 8.6 Hz, 2 H), 7.05 (d, J = 8.6 Hz, 2 H), 7.53 (s, 1 H), 7.68 (d, J = 0.9 Hz, 1 H), 7.78 (s, 1 H), 7.93 (s, 1 H), 8.04 (d, J = 0.9 Hz, 1 H), 8.21 (s, 1 H), 8.30 (s, 1 H), 9.78 (s, 1 H), 10.77 (s, 1 H). Three protons not apparent in the ¹H NMR spectrum. |
| TBM-2 | CBM-22 | 76 | 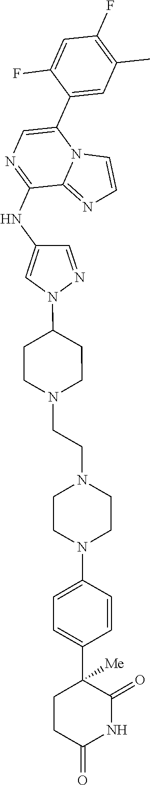 [SMILES: Cc1cc(-c2cnc(Nc3cnn(C4CCN(CCN5CCN(c6ccc([C@]7(C)CCC(=O)NC7=O)cc6)CC5)CC4)c3)c3nccn23)c(F)cc1F] | (S)-3-(4-(4-(2-(4- (4-((5-(2,4- difluoro-5- methylphenyl) imdazo [1,2-a]pyrazin- 8-yl)amino)-1H- pyrazol-1- yl)piperidin-1- yl)ethyl)piperazin- 1-yl)phenyl)-3- methylpiperidine- 2,6-dione | 63 mg, 39% yield (3 steps), white solid [M + H]]⁺ = 723.4 ¹H NMR (400 MHz, DMSO- d6 ) δ ppm 1.38 (s, 3 H), 1.87-2.10 (m, 7 H), 2.11- 2.20 (m, 2 H), 2.27-2.34 (m, 4 H), 2.40-2.45 (m, 1 H), 2.53-2.59 (m, 5 H), 3.02 (br d, J = 11.5 Hz, 2 H), 3.09-3.16 (m, 5 H), 4.11- 4.18 (m, 1 H), 6.91 (d, J = 8.8 Hz, 2 H), 7.10 (d, J = 8.8 Hz, 2 H), 7.42-7.48 (m, 2 H), 7.58-7.64 (m, 3 H), 7.80 (s, 1 H), 8.23 (s, 1 H), 9.98 (s, 1 H), 10.84 (s, 1 H). ¹⁹F NMR (377 MHz, DMSO-d6) δ ppm −112.24- −112.09 (m, 1 F), −111.30- −111.20 (m, 1 F). |
| TBM-2 | CBM-23 | 77 | 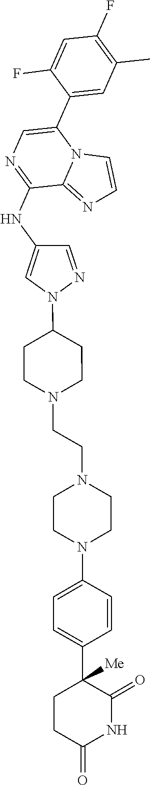 [SMILES: Cc1cc(-c2cnc(Nc3cnn(C4CCN(CCN5CCN(c6ccc([C@@]7(C)CCC(=O)NC7=O)cc6)CC5)CC4)c3)c3nccn23)c(F)cc1F] | (R)-3-(4-(4-(2-(4- (4-((5-(2,4- difluoro-5- methylphenyl) imdazo [1,2-a]pyrazin- 8-yl)amino)-1H- pyrazol-1- yl)piperidin-1- yl)ethyl)piperazin- 1-yl)phenyl)-3- methylpiperidine- 2,6-dione | 57 mg, 26% yield (3 steps), white solid [M + H]]⁺ = 723.3 ¹H NMR (400 MHz, DMSO- d6 ) δ ppm 1.38 (s, 3 H), 1.87-2.02 (m, 4 H), 2.02- 2.08 (m, 2 H), 2.09-2.18 (m, 2 H), 2.26-2.34 (m, 4 H), 2.39-2.44 (m, 1 H), 2.53-2.59 (m, 4 H), 3.00 (br d, J = 11.2 Hz, 2 H), 3.08- 3.16 (m, 4 H), 4.09-4.17 (m, 1 H), 6.91 (d, J = 8.8 Hz, 2 H), 7.10 (d, J = 8.8 Hz, 2 H), 7.42-7.48 (m, 2 H), 7.58-7.65 (m, 3 H), 7.80 (s, 1 H), 8.23 (s, 1 H), 9.98 (s, 1 H), 10.84 (s, 1 H). ¹⁹F NMR (377 MHz, DMSO-d6) δ ppm −112.21- −112.10 (m, 1 F), −111.30- −111.19 (m, 1 F). |
| TBM-2 | CBM-25 | 97 | 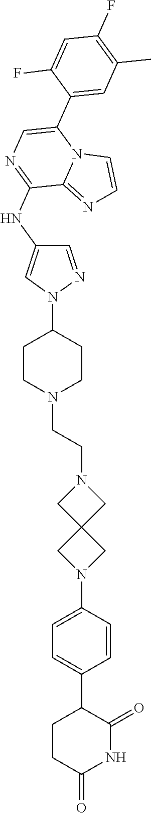 [SMILES: Cc1cc(-c2cnc(Nc3cnn(C4CCN(CCN5CC6(C5)CN(c5ccc(C7CCC(=O)NC7=O)cc5)C6)CC4)c3)c3nccn23)c(F)cc1F] | 3-(4-(6-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)-2,6- diazaspiro[3.3] heptan-2- yl)phenyl) piperidine- 2,6-dione | 36 mg, 15% yield (3 steps), white solid [M + H]]⁺ = 721.3. ¹H NMR (400 MHz, DMSO- d6) δ ppm 1.84-2.15 (m, 9 H), 2.24-2.36 (m, 5 H), 2.56-2.68 (m, 2 H), 2.89- 2.99 (m, 2 H), 3.67 (dd, J = 10.8, 4.6 Hz, 1 H), 3.78- 3.88 (m, 4 H), 4.05-4.16 (m, 1 H), 6.34 (d, J = 8.1 Hz, 2 H), 6.96 (d, J = 8.1 Hz, 2 H), 7.40-7.49 (m, 2 H), 7.57-7.64 (m, 3 H), 7.80 (s, 1 H), 8.22 (s, 1 H), 9.97 (s, 1 H), 10.74 (s, 1 H). ¹⁹F NMR (377 MHz, DMSO-d6) δ ppm −112.17 (d, J = 1.0 Hz, 1 F), −111.26 (d, J = 1.0 Hz, 1 F). |
| TBM-23 | CBM-2 | 98 | 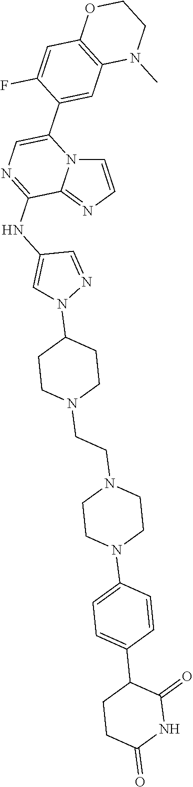 [SMILES: CN1CCOc2cc(F)c(-c3cnc(Nc4cnn(C5CCN(CCN6CCN(c7ccc(C8CCC(=O)NC8=O)cc7)CC6)CC5)c4)c4nccn34)cc21] | 3-(4-(4-(2-(4-(4- ((5-(7-fluoro-4- methyl-3,4- dihydro-2H- benzo[b][1,4] oxazin-6- yl)imidazo[1,2- a]pyrazin-8- yl)amino)-1H- pyrazol-1- yl)piperidin-1- yl)ethyl)piperazin- 1- yl)phenyl) piperidine- 2,6-dione | 13 mg, 11% yield (3 steps), white solid [M + H]]⁺ = 748.4 ¹H NMR (400 MHz, DMSO- d6) δ ppm 1.84-2.05 (m, 5 H), 2.07-2.19 (m, 3 H), 2.42-2.48 (m, 2 H), 2.53- 2.68 (m, 6 H), 2.84 (s, 3 H), 3.00 (br d, J = 11.2 Hz, 2 H), 3.07-3.14 (m, 4 H), 3.24- 3.30 (m, 3 H), 3.72 (dd, J = 11.1, 5.0 Hz, 1 H), 4.08- 4.17 (m, 1 H), 4.30-4.37 (m, 2 H), 6.77-6.86 (m, 2 H), 6.89 (d, J = 8.8 Hz, 2 H), 7.04 (d, J = 8.8 Hz, 2 H), 7.41 (s, 1 H), 7.56 (dd, J = 3.1, 1.1 Hz, 1 H), 7.60 (d, J = 1.2 Hz, 1 H), 7.79 (s, 1 H), 8.24 (s, 1 H), 9.89 (s, 1 H), 10.77 (s, 1 H). ¹⁹F NMR (377 MHz, DMSO-d6) δ ppm −124.71- −124.53 (m, 1 F). |
| TBM-2 | CBM-26 | 102 | 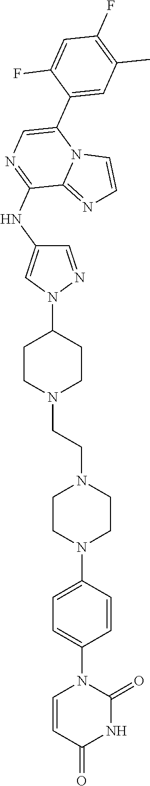 [SMILES: Cc1cc(-c2cnc(Nc3cnn(C4CCN(CCN5CCN(c6ccc(-n7ccc(=O)[nH]c7=O)cc6)CC5)CC4)c3)c3nccn23)c(F)cc1F] | 1-(4-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1- yl)phenyl)pyrimidi ne-2,4(1H,3H)- dione | 29 mg, 10% yield (3 steps), off-white solid [M + H]]⁺ = 708.3 ¹H NMR (400 MHz, DMSO- d6) δ ppm 1.80-2.05 (m, 5 H), 2.14 (br t, J = 11.2 Hz, 2 H), 2.29 (s, 3 H), 2.58 (br s, 5 H), 3.01 (br d, J = 11.5 Hz, 2 H), 3.12-3.28 (m, 6 H), 4.08-4.21 (m, 1 H), 5.61 (d, J = 7.8 Hz, 1 H), 6.99 (d, J = 9.0 Hz, 2 H), 7.21 (d, J = 8.8 Hz, 2 H), 7.40-7.50 (m, 2 H), 7.58-7.65 (m, 4 H), 7.80 (s, 1 H), 8.23 (s, 1 H), 9.98 (s, 1 H), 11.35 (s, 1 H). ¹⁹F NMR (377 MHz, DMSO-d6) δ ppm −112.17 (q, J = 7.3 Hz, 1 F), −111.30- −111.19 (m, 1 F). |
| TBM-2 | CBM-29 | 109 | 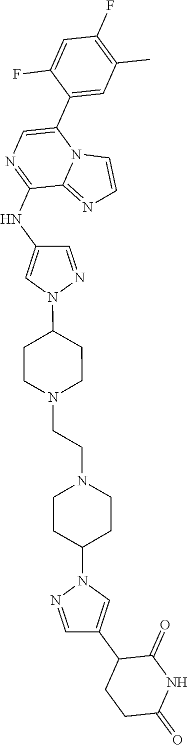 [SMILES: Cc1cc(-c2cnc(Nc3cnn(C4CCN(CCN5CCC(n6cc(C7CCC(=O)NC7=O)cn6)CC5)CC4)c3)c3nccn23)c(F)cc1F] | 3-(1-(1-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperidin- 4-yl)-1H-pyrazol- 4-yl)piperidine- 2,6-dione | 39 mg, 17% yield (3 steps), white solid [M + H]]⁺ = 349.8; [M + H]]⁺ = 698.3 ¹H NMR (400 MHz, DMSO- d6) δ ppm 1.80-2.20 (m, 15 H), 2.29 (s, 3 H), 2.46-2.48 (m, 4 H), 2.55-2.66 (m, 1 H), 2.98 (br d, J = 7.1 Hz, 4 H), 3.73 (dd, J = 10.0, 5.1 Hz, 1 H), 4.03-4.17 (m, 2 H), 7.36 (s, 1 H), 7.42-7.48 (m, 2 H), 7.58-7.64 (m, 3 H), 7.69 (s, 1 H), 7.80 (s, 1 H), 8.23 (s, 1H), 9.98 (s, 1 H), 10.73 (s, 1 H). ¹⁹F NMR (377 MHz, DMSO-d6) δ ppm −112.09 (q, J = 8.2 Hz, 1 F), −111.18 (q, J = 6.8 Hz, 1 F). |
| TBM-2 | CBM-30 | 110 | 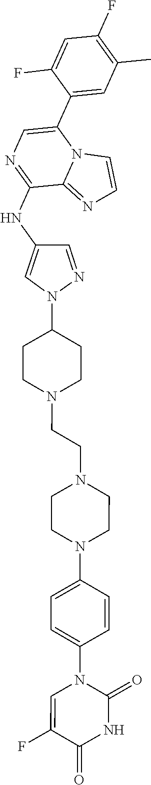 [SMILES: Cc1cc(-c2cnc(Nc3cnn(C4CCN(CCN5CCN(c6ccc(-n7cc(F)c(=O)[nH]c7=O)cc6)CC5)CC4)c3)c3nccn23)c(F)cc1F] | 1-(4-(4-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperazin- 1-yl)phenyl)-5- fluoropyrimidine- 2,4(1H,3H)-dione | 7 mg, 6% yield (3 steps), white solid [M + H]]⁺ = 726.3 ¹H NMR (400 MHz, DMSO- d6) δ ppm 1.83-2.05 (m, 4 H), 2.08-2.18 (m, 2 H), 2.29 (s, 3 H), 2.52-2.61 (m, 4 H), 2.95-3.05 (m, 2 H), 3.13-3.25 (m, 6 H), 4.08- 4.21 (m, 1 H), 6.90-7.06 (m, 3 H), 7.23 (d, J = 9.0 Hz, 2 H), 7.41-7.50 (m, 2 H), 7.57-7.67 (m, 3 H), 7.77- 7.85 (m, 1 H), 8.06 (br d, J = 6.6 Hz, 1 H), 8.23 (s, 1 H), 8.50 (s, 2 H), 9.98 (s, 1 H). ¹⁹F NMR (377 MHz, DMSO-d6) δ ppm −170.13- −169.56 (m, 1 F), −112.16 (q, J = 9.1 Hz, 1 F), −111.25 (q, J = 8.2 Hz, 1 F). |
| TBM-7 | CBM-11 | 113 | 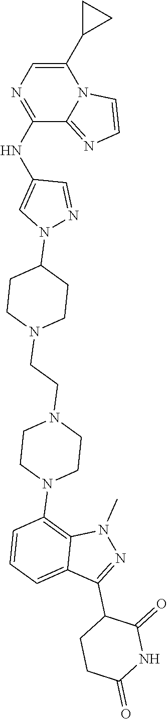 [SMILES: Cn1nc(C2CCC(=O)NC2=O)c2cccc(N3CCN(CCN4CCC(n5cc(Nc6ncc(C7CC7)n7ccnc67)cn5)CC4)CC3)c21] | 3-(7-(4-(2-(4-(4- ((5- cyclopropylimidaz o[1,2-a]pyrazin-8- yl)amino)-1H- pyrazol-1- yl)piperidin-1- yl)ethyl)piperazin- 1-yl)-1-methyl- 1H-indazol-3- yl)piperidine-2,6- dione | 38 mg, 17% yield (3 steps), white solid [M + H]]⁺ = 677.3. ¹H NMR (400 MHz, DMSO- d6 ) δ ppm 0.71-0.75 (m, 2 H), 0.98-1.02 (m, 2 H), 1.88-2.07 (m, 6 H), 2.12- 2.20 (m, 3 H), 2.27-2.41 (m, 3 H), 2.53-2.58 (m, 3 H), 2.59-2.71 (m, 2 H), 2.85-3.19 (m, 8 H), 4.07- 4.15 (m, 1 H), 4.25 (s, 3 H), 4.34 (dd, J = 9.5, 5.1 Hz, 1 H), 7.00-7.06 (m, 2 H), 7.22 (d, J = 1.0 Hz, 1 H), 7.39 (dd, J = 7.3, 1.5 Hz, 1 H), 7.66 (d, J = 1.0 Hz, 1 H), 7.74 (s, 1 H), 8.05 (d, J = 1.0 Hz, 1 H), 8.18 (s, 1 H), 9.65 (s, 1 H), 10.88 (s, 1 H). |
| TBM-7 | CBM-10 | 115 | 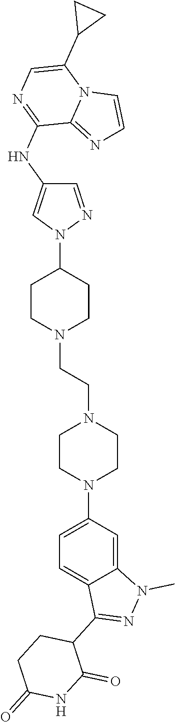 [SMILES: Cn1nc(C2CCC(=O)NC2=O)c2ccc(N3CCN(CCN4CCC(n5cc(Nc6ncc(C7CC7)n7ccnc67)cn5)CC4)CC3)cc21] | 3-(6-(4-(2-(4-(4- ((5- cyclopropylimidaz o[1,2-a]pyrazin-8- yl)amino)-1H- pyrazol-1- yl)piperidin-1- yl)ethyl)piperazin- 1-yl)-1-methyl- 1H-indazol-3- yl)piperidine-2,6- dione | 16 mg, 7% yield (3 steps), off-white solid [M + H]]⁺ = 677.4. ¹H NMR (400 MHz, DMSO- d6 ) δ ppm 0.73 (br d, J = 4.4 Hz, 2 H), 1.00 (br d, J = 7.8 Hz, 1 H), 1.83-2.07 (m, 6 H), 2.10-2.20 (m, 3 H), 2.23-2.37 (m, 2 H), 2.59- 2.66 (m, 6 H), 3.01 (br d, J = 10.5 Hz, 2 H), 3.20-3.25 (m, 4 H), 3.89 (s, 3 H), 4.06- 4.15 (m, 1 H), 4.26 (dd, J = 8.7, 5.0 Hz, 1 H), 6.84 (s, 1 H), 6.92 (br d, J = 9.3 Hz, 1 H), 7.22 (s, 1 H), 7.50 (d, J = 9.0 Hz, 1 H), 7.65 (s, 1 H), 7.74 (s, 1 H), 8.05 (s, 1 H), 8.18 (s, 1 H), 9.64 (s, 1 H), 10.85 (s, 1 H). |
| TBM-2 | CBM-31 | 116 | 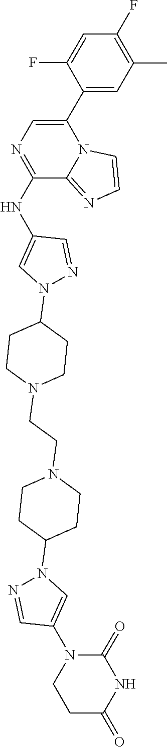 [SMILES: Cc1cc(-c2cnc(Nc3cnn(C4CCN(CCN5CCC(n6cc(N7CCC(=O)NC7=O)cn6)CC5)CC4)c3)c3nccn23)c(F)cc1F] | 1-(1-(1-(2-(4-(4- ((5-(2,4-difluoro- 5- methylphenyl) imdazo [1,2-a]pyrazin- 8-yl)amino)-1H- pyrazol-1- yl)piperidin-1- yl)ethyl)piperidin- 4-yl)-1H-pyrazol- 4- yl) dihydropyrimidine- 2,4(1H,3H)- dione | 40 mg, 16% yield (3 steps), white solid [M + 2H]²⁺ = 350.8; [M + H]⁺ = 699.3 ¹H NMR (400 MHz, DMSO- d6) δ ppm 1.84-2.02 (m, 9 H), 2.07-2.18 (m, 4 H), 2.29 (s, 3 H), 2.68 (t, J = 6.8 Hz, 2 H), 2.94-3.04 (m, 4 H), 3.74 (t, J = 6.8 Hz, 2 H), 4.04-4.18 (m, 2 H), 7.42- 7.49 (m, 2 H), 7.58-7.64 (m, 4 H), 7.80 (s, 1 H), 7.94 (s, 1 H), 8.23 (s, 1 H), 10.00 (s, 1 H), 10.37 (s, 1 H). ¹⁹F NMR (377 MHz, DMSO-d6) δ ppm −112.15 (q, J = 8.2 Hz, 1 F), −111.25 (q, J = 8.2 Hz, 1 F). |
| TBM-27 | CBM-2 | 119 | 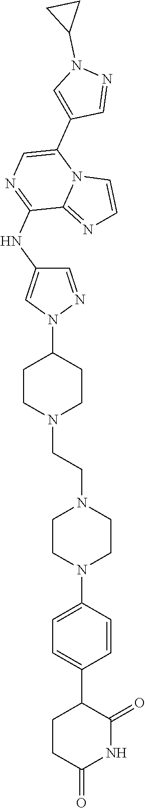 [SMILES: O=C1CCC(c2ccc(N3CCN(CCN4CCC(n5cc(Nc6ncc(-c7cnn(C8CC8)c7)n7ccnc67)cn5)CC4)CC3)cc2)C(=O)N1] | 3-(4-(4-(2-(4-(4- ((5-(1- cyclopropyl-1H- pyrazol-4- yl)imidazo[1,2- a]pyrazin-8- yl)amino)-1H- pyrazol-1- yl)piperidin-1- yl)ethyl)piperazin- 1- yl)phenyl) piperidine- 2,6-dione | 45 mg, 12% yield (3 steps), off-white solid [M + H]⁺ = 689.4. ¹H NMR (400 MHz, DMSO- d6) δ ppm 0.99-1.06 (m, 2 H), 1.13-1.19 (m, 2 H), 1.86-2.05 (m, 5 H), 2.07- 2.20 (m, 3 H), 2.41-2.48 (m, 2 H), 2.53-2.67 (m, 6 H), 2.98-3.05 (m, 2 H), 3.09-3.15 (m, 4 H), 3.72 (dd, J = 10.9, 5.0 Hz, 1 H), 3.82 (spt, J = 3.8 Hz, 1 H), 4.07-4.17 (m, 1 H), 6.89 (d, J = 8.6 Hz, 2 H), 7.05 (d, J = 8.6 Hz, 2 H), 7.55 (s, 1 H), 7.68 (br s, 1 H), 7.78 (s, 1 H), 7.93 (s, 1 H), 8.08 (br s, 1 H), 8.21 (s, 1 H), 8.37 (s, 1 H), 9.78 (s, 1 H), 10.77 (s, 1 H). |

### Example S22. Representative Procedure for LDD-K

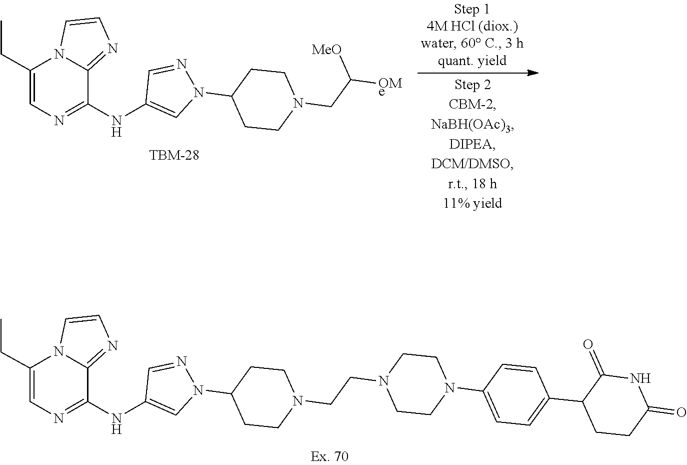 [SMILES: CCc1cnc(Nc2cnn(C3CCN(CC(OC)OC)CC3)c2)c2nccn12 ; CCc1cnc(Nc2cnn(C3CCN(CCN4CCN(c5ccc(C6CCC(=O)NC6=O)cc5)CC4)CC3)c2)c2nccn12]

Step 1: To a round bottom flask containing N-[1-[1-(2,2-dimethoxyethyl)-4-piperidyl]pyrazol-4-yl]-5-ethyl-imidazo[1,2-a]pyrazin-8-amine TBM-28 (30.0 mg, 0.07 mmol) were added 4M HCl in 1,4-dioxane (254 μL, 1.01 mmol, 15 eq.) and water (0.10 mL) at room temperature. The reaction mixture was heated to 60° C. and stirred for 3 h. The solvents were removed under reduced pressure and excess HCl was chased with MeCN (4×) to afford 2-(4-(4-((5-ethylimidazo[1,2-a]pyrazin-8-yl)amino)-1H-pyrazol-1-yl)piperidin-1-yl)acetaldehyde (26 mg, quant.) as a yellow solid, which was directly used in the following step.

Step 2: To a solution of 2-(4-(4-((5-ethylimidazo[1,2-a]pyrazin-8-yl)amino)-1H-pyrazol-1-yl)piperidin-1-yl)acetaldehyde (27. mg, 0.07 mmol, 1 eq.) and DIPEA (0.06 mL, 0.35 mmol, 5 eq.) in CH₂Cl₂ (0.69 mL) were added 3-(4-piperazin-1-ylphenyl)piperidine-2,6-dione dihydrochloride (29 mg, 0.08 mmol, 1.2 eq.) and NaBH(OAc)₃ (29 mg, 0.14 mmol, 2 eq.). The reaction mixture was stirred at room temperature for 18 h. The solvent was evaporated and the residue was purified by reverse phase column chromatography (5% to 100% MeCN in water w/0.1% formic acid). The fractions were combined, concentrated, and purified again by reverse phase column chromatography (500 to 10000 MeCN in water). The fractions were combined, concentrated, and lyophilised to afford 3-(4-(4-(2-(4-(4-((5-ethylimidazo[1,2-a]pyrazin-8-yl)amino)-1H-pyrazol-1-yl)piperidin-1-yl)ethyl)piperazin-1-yl)phenyl)piperidine-2,6-dione Ex. 70 (5 mg, 11% yield) as an off-white solid.

LCMS: [M+H]⁺=611.4.

¹H NMR (400 MHz, DMSO-d₆) δ ppm 1.29 (t, J=7.3 Hz, 3H), 1.84-2.05 (m, 6H), 2.07-2.19 (m, 3H), 2.41-2.47 (m, 3H), 2.52-2.65 (m, 6H), 2.84 (q, J=7.6 Hz, 2H), 3.00 (br d, J=11.7 Hz, 2H), 3.09-3.13 (m, 4H), 3.72 (dd, J=10.8, 4.6 Hz, 1H), 4.04-4.16 (m, 1H), 6.89 (d, J=8.8 Hz, 2H), 7.05 (d, J=8.8 Hz, 2H), 7.24 (s, 1H), 7.64 (d, J=1.2 Hz, 1H), 7.74-7.75 (m, 1H), 7.96 (d, J=1.0 Hz, 1H), 8.18 (s, 1H), 8.19 (s, 1H), 9.58 (s, 1H), 10.77 (s, 1H).

TABLE 37
List of Examples prepared by procedure LDD-K

| Components | | | | | |
|---|---|---|---|---|---|
| TBM | CBM | Example | | | |
| # | # | Cmp | Structure | IUPAC Name | Characterization |
| TBM-28 | CBM-2 | 70 | 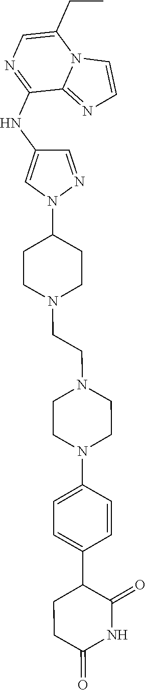 [SMILES: CCc1cnc(Nc2cnn(C3CCN(CCN4CCN(c5ccc(C6CCC(=O)NC6=O)cc5)CC4)CC3)c2)c2nccn12] | 3-(4-(4-(2-(4-(4- ((5- ethylimidazo[1,2- a]pyrazin-8- yl)amino)-1H- pyrazol-1- yl)piperidin-1- yl)ethyl)piperazin- 1- yl)phenyl) piperidine- 2,6-dione | 5 mg, 11% yield (2 steps), off- white solid [M + H]⁺ = 611.4 ¹H NMR (400 MHz, DMSO-d6) δ ppm 1.29 (t, J = 7.3 Hz, 3 H), 1.84-2.05 (m, 6 H), 2.07-2.19 (m, 3 H), 2.41-2.47 (m, 3 H), 2.52-2.65 (m, 6 H), 2.84 (q, J = 7.6 Hz, 2 H), 3.00 (br d, J = 11.7 Hz, 2 H), 3.09-3.13 (m, 4 H), 3.72 (dd, J = 10.8, 4.6 Hz, 1 H), 4.04-4.16 (m, 1 H), 6.89 (d, J = 8.8 Hz, 2 H), 7.05 (d, J = 8.8 Hz, 2 H), 7.24 (s, 1 H), 7.64 (d, J = 1.2 Hz, 1 H), 7.74-7.75 (m, 1 H), 7.96 (d, J = 1.0 Hz, 1 H), 8.18 (s, 1 H), 8.19 (s, 1 H), 9.58 (s, 1 H), 10.77 (s, 1 H). |
| TBM-29 | CBM-2 | 74 | 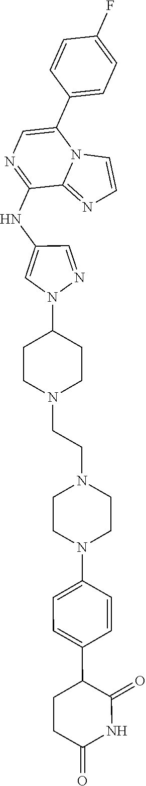 [SMILES: O=C1CCC(c2ccc(N3CCN(CCN4CCC(n5cc(Nc6ncc(-c7ccc(F)cc7)n7ccnc67)cn5)CC4)CC3)cc2)C(=O)N1] | 3-(4-(4-(2-(4-(4- ((5-(4- fluorophenyl) imidazo [1,2-a]pyrazin- 8-yl)amino)-1H- pyrazol-1- yl)piperidin-1- yl)ethyl)piperazin- 1- yl)phenyl) piperidine- 2,6-dione | 14 mg, 13% yield (2 steps), white solid [M + H]⁺ = 677.3. ¹H NMR (400 MHz, DMSO-d6) δ ppm 1.92-2.08 (m, 3 H), 2.09- 2.23 (m, 4 H), 2.41-2.48 (m, 4 H), 2.53-2.57 (m, 1 H), 2.58- 2.65 (m, 1 H), 2.70-2.87 (m, 4 H), 3.14-3.23 (m, 7 H), 3.73 (dd, J = 10.9, 4.8 Hz, 1 H), 4.25-4.48 (m, 1 H), 6.92 (d, J = 8.6 Hz, 2 H), 7.06 (d, J = 8.8 Hz, 2 H), 7.39- 7.43 (m, 3 H), 7.65 (d, J = 1.1 Hz, 1 H), 7.72-7.77 (m, 2 H), 7.82 (s, 1 H), 7.85 (d, J = 1.0 Hz, 1 H), 8.27 (s, 1 H), 9.95 (s, 1 H), 10.77 (s, 1 H). ¹⁹F NMR (377 MHz, DMSO-d6) δ ppm −112.37 (br s, 1 F). |
| TBM-30 | CBM-2 | 75 | 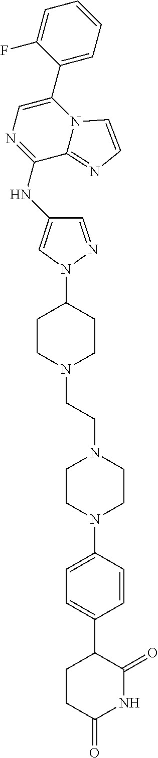 [SMILES: O=C1CCC(c2ccc(N3CCN(CCN4CCC(n5cc(Nc6ncc(-c7ccccc7F)n7ccnc67)cn5)CC4)CC3)cc2)C(=O)N1] | 3-(4-(4-(2-(4-(4- ((5-(2- fluorophenyl) imidazo [1,2-a]pyrazin- 8-yl)amino)-1H- pyrazol-1- yl)piperidin-1- yl)ethyl)piperazin- 1- yl)phenyl) piperidine- 2,6-dione | 56 mg, 23% yield (2 steps), white solid [M + H]⁺ = 677.3. ¹H NMR (400 MHz, DMSO-d6) δ ppm 1.91-2.04 (m, 5 H), 2.10- 2.23 (m, 3 H), 2.42-2.48 (m, 2 H), 2.53-2.55 (m, 3 H), 2.56- 2.60 (m, 4 H), 2.62 (br d, J = 6.2 Hz, 1 H), 3.04 (br d, J = 11.7 Hz, 2 H), 3.09-3.14 (m, 4 H), 3.72 (br dd, J = 10.7, 4.8 Hz, 1 H), 4.10-4.22 (m, 1 H), 6.89 (d, J = 8.7 Hz, 2 H), 7.05 (d, J = 8.7 Hz, 2 H), 7.39-7.49 (m, 3 H), 7.57- 7.69 (m, 4 H), 7.81 (s, 1 H), 8.15 (s, 2 H), 8.25 (s, 1 H), 9.99 (s, 1 H), 10.77 (s, 1 H). 2 H not observed. ¹⁹F NMR (377 MHz, DMSO-d6) δ ppm −112.16 (br s, 1 F). |
| TBM-31 | CBM-2 | 78 | 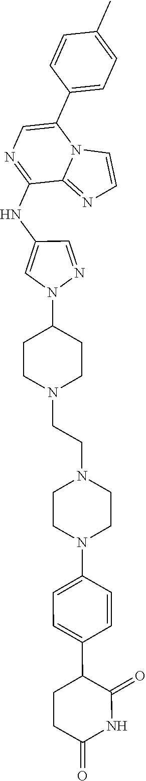 [SMILES: Cc1ccc(-c2cnc(Nc3cnn(C4CCN(CCN5CCN(c6ccc(C7CCC(=O)NC7=O)cc6)CC5)CC4)c3)c3nccn23)cc1] | 3-(4-(4-(2-(4-(4- ((5-(p- tolyl)imidazo[1,2- a]pyrazin-8- yl)amino)-1H- pyrazol-1- yl)piperidin-1- yl)ethyl)piperazin- 1- yl)phenyl) piperidine- 2,6-dione | 57 mg, 32% yield (2 steps), white solid [M + H]⁺ = 673.3; [M + 2H]²⁺ = 337.2. ¹H NMR (400 MHz, DMSO-d6) δ ppm 1.88-2.05 (m, 5 H), 2.06- 2.26 (m, 3 H), 2.40 (s, 3 H), 2.53- 2.69 (m, 10 H), 3.00-3.08 (m, 2 H), 3.09-3.16 (m, 4 H), 3.72 (dd, J = 10.9, 4.5 Hz, 1 H), 4.09-4.20 (m, 1 H), 6.89 (d, J = 8.4 Hz, 2 H), 7.05 (d, J = 8.6 Hz, 2 H), 7.32- 7.44 (m, 3 H), 7.57 (d, J = 7.8 Hz, 2 H), 7.64 (s, 1 H), 7.79 (s, 1 H), 7.86 (s, 1 H), 8.24 (s, 1 H), 9.87 (s, 1 H), 10.77 (s, 1 H). |
| TBM-32 | CBM-2 | 79 | 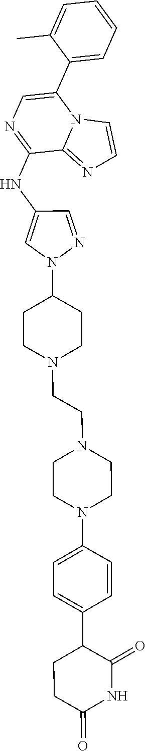 [SMILES: Cc1ccccc1-c1cnc(Nc2cnn(C3CCN(CCN4CCN(c5ccc(C6CCC(=O)NC6=O)cc5)CC4)CC3)c2)c2nccn12] | 3-(4-(4-(2-(4-(4- ((5-(o- tolyl)imidazo[1,2- a]pyrazin-8- yl)amino)-1H- pyrazol-1- yl)piperidin-1- yl)ethyl)piperazin- 1- yl)phenyl) piperidine- 2,6-dione | 56 mg, 26% yield (2 steps) white solid [M + H]⁺ = 673.4 ¹H NMR (400 MHz, DMSO-d6) δ ppm 1.87-2.05 (m, 5 H), 2.07- 2.19 (m, 6 H), 2.46-2.48 (m, 1 H), 2.54-2.69 (m, 6 H), 3.01 (br d, J = 11.5 Hz, 2 H), 3.11 (m, 4 H), 3.72 (dd, J = 11.0, 4.9 Hz, 1 H), 4.08-4.20 (m, 1 H), 6.89 (d, J = 8.6 Hz, 2 H), 7.04 (d, J = 8.6 Hz, 2 H), 7.27 (d, J = 1.0 Hz, 1 H), 7.33 (s, 1 H), 7.35-7.50 (m, 4 H), 7.59 (d, J = 1.0 Hz, 1 H), 7.77-7.82 (m, 1 H), 8.25 (s, 1 H), 9.87 (s, 1 H), 10.77 (s, 1 H). |
| TBM-33 | CBM-2 | 80 | 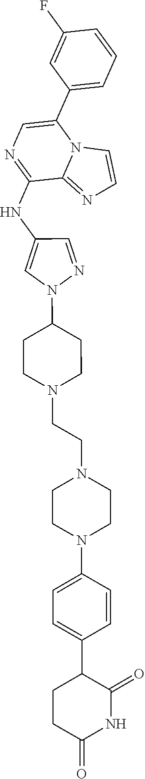 [SMILES: O=C1CCC(c2ccc(N3CCN(CCN4CCC(n5cc(Nc6ncc(-c7cccc(F)c7)n7ccnc67)cn5)CC4)CC3)cc2)C(=O)N1] | 3-(4-(4-(2-(4-(4- ((5-(3- fluorophenyl) imidazo [1,2-a]pyrazin- 8-yl)amino)-1H- pyrazol-1- yl)piperidin-1- yl)ethyl)piperazin- 1- yl)phenyl) piperidine- 2,6-dione | 15 mg, 6% yield (2 steps), white solid [M + H]⁺ = 677.3. ¹H NMR (400 MHz, DMSO-d6) δ ppm 1.86-2.04 (m, 6 H), 2.06- 2.21 (m, 4 H), 2.55-2.63 (m, 6 H), 2.98-3.06 (m, 3 H), 3.07- 3.15 (m, 5 H), 3.69-3.76 (m, 1 H), 4.08-4.20 (m, 1 H), 6.89 (br d, J = 7.6 Hz, 2 H), 7.05 (br d, J = 8.1 Hz, 2 H), 7.30-7.38 (m, 1 H), 7.50 (s, 1 H), 7.52-7.63 (m, 3 H), 7.66 (br s, 1 H), 7.80 (s, 1 H), 7.95 (s, 1 H), 8.24 (s, 1 H), 9.97 (br s, 1 H), 10.77 (br s, 1 H). ¹⁹F NMR (377 MHz, DMSO-d6) δ ppm −111.82 (br s, 1 F). |
| TBM-34 | CBM-2 | 83 | 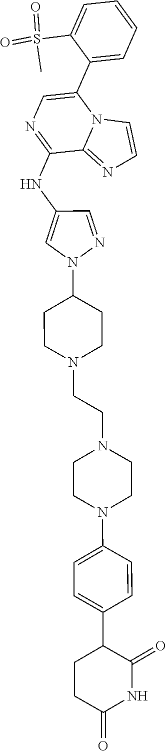 [SMILES: CS(=O)(=O)c1ccccc1-c1cnc(Nc2cnn(C3CCN(CCN4CCN(c5ccc(C6CCC(=O)NC6=O)cc5)CC4)CC3)c2)c2nccn12] | 3-(4-(4-(2-(4-(4- ((5-(2- (methylsulfonyl) phenyl)imidazo[1,2- a]pyrazin-8- yl)amino)-1H- pyrazol-1- yl)piperidin-1- yl)ethyl)piperazin- 1- yl)phenyl) piperidine- 2,6-dione | 19 mg, 12% yield (2 steps), white solid [M + H]⁺ = 737.4. ¹H NMR (400 MHz, DMSO-d6) δ ppm 1.86-2.06 (m, 5 H), 2.07- 2.19 (m, 3 H), 2.41-2.48 (m, 2 H), 2.53-2.66 (m, 6 H), 3.01 (br d, J = 11.0 Hz, 2 H), 3.07-3.14 (m, 6 H), 3.72 (dd, J = 11.0, 4.9 Hz, 1 H), 4.13 (ddd, J = 15.5, 11.0, 4.3 Hz, 1 H), 6.88 (d, J = 8.8 Hz, 2 H), 7.04 (d, J = 8.8 Hz, 2 H), 7.15 (d, J = 1.2 Hz, 1 H), 7.43 (s, 1 H), 7.52 (d, J = 1.2 Hz, 1 H), 7.69 (dd, J = 7.3, 1.5 Hz, 1 H), 7.79 (s, 1 H), 7.85-7.95 (m, 2 H), 8.20 (dd, J = 7.7, 1.6 Hz, 1 H), 8.25 (s, 1 H), 9.93 (s, 1 H), 10.77 (s, 1 H). |
| TBM-35 | CBM-2 | 85 | 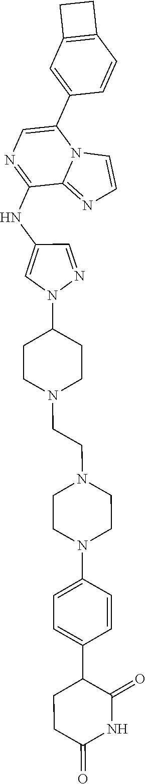 [SMILES: O=C1CCC(c2ccc(N3CCN(CCN4CCC(n5cc(Nc6ncc(-c7ccc8c(c7)CC8)n7ccnc67)cn5)CC4)CC3)cc2)C(=O)N1] | 3-(4-(4-(2-(4-(4- ((5- (bicyclo[4.2.0]octa- 1(6),2,4-trien-3- yl)imidazo[1,2- a]pyrazin-8- yl)amino)-1H- pyrazol-1- yl)piperidin-1- yl)ethyl)piperazin- 1- yl)phenyl) piperidine- 2,6-dione | 23 mg, 9% yield (2 steps), white solid [M + H]⁺ = 685.4 ¹H NMR (400 MHz, DMSO-d6) δ ppm 1.85-2.05 (m, 5 H), 2.06- 2.20 (m, 3 H), 2.41-2.45 (m, 1 H), 2.54-2.65 (m, 6 H), 2.97- 3.05 (m, 2 H), 3.08-3.14 (m, 4 H), 3.23 (s, 4 H), 3.72 (dd, J = 11.0, 4.9 Hz, 1 H), 4.07-4.20 (m, 1 H), 6.89 (d, J = 8.8 Hz, 2 H), 7.05 (d, J = 8.8 Hz, 2 H), 7.27 (d, J = 7.6 Hz, 1 H), 7.34-7.39 (m, 2 H), 7.44-7.49 (m, 1 H), 7.62 (d, J = 1.0 Hz, 1 H), 7.79 (s, 1 H), 7.82 (d, J = 1.2 Hz, 1 H), 8.24 (s, 1 H), 9.84 (s, 1 H), 10.77 (s, 1 H). |
| TBM-36 | CBM-2 | 86 | 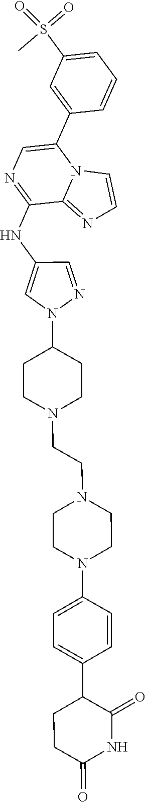 [SMILES: CS(=O)(=O)c1cccc(-c2cnc(Nc3cnn(C4CCN(CCN5CCN(c6ccc(C7CCC(=O)NC7=O)cc6)CC5)CC4)c3)c3nccn23)c1] | 3-(4-(4-(2-(4-(4- ((5-(3- (methylsulfonyl) phenyl)imidazo[1,2- a]pyrazin-8- yl)amino)-1H- pyrazol-1- yl)piperidin-1- yl)ethyl)piperazin- 1- yl)phenyl) piperidine- 2,6-dione | 20 mg, 8% yield (2 steps), light- yellow solid [M + H]⁺ = 737.3. ¹H NMR (400 MHz, DMSO-d6) δ ppm 1.89-2.05 (m, 5 H), 2.07- 2.23 (m, 3 H), 2.42-2.48 (m, 2 H), 2.53-2.67 (m, 9 H), 3.04 (br d, J = 11.2 Hz, 2 H), 3.09-3.14 (m, 4 H), 3.72 (dd, J = 11.1, 5.0 Hz, 1 H), 4.10-4.20 (m, 1 H), 6.89 (d, J = 8.8 Hz, 2 H), 7.05 (d, J = 8.8 Hz, 2 H), 7.58 (s, 1 H), 7.69 (d, J = 1.2 Hz, 1 H), 7.81- 7.87 (m, 2 H), 7.97 (d, J = 1.2 Hz, 1 H), 8.01-8.09 (m, 2 H), 8.18- 8.21 (m, 1 H), 8.25 (s, 1 H), 10.05 (s, 1 H), 10.77 (s, 1 H). |
| TBM-37 | CBM-2 | 87 | 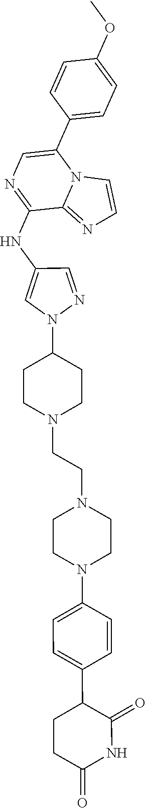 [SMILES: COc1ccc(-c2cnc(Nc3cnn(C4CCN(CCN5CCN(c6ccc(C7CCC(=O)NC7=O)cc6)CC5)CC4)c3)c3nccn23)cc1] | 3-(4-(4-(2-(4-(4- ((5-(4- methoxyphenyl) imidazo[1,2- a]pyrazin-8- yl)amino)-1H- pyrazol-1- yl)piperidin-1- yl)ethyl)piperazin- 1- yl)phenyl) piperidine- 2,6-dione | 31 mg, 12% yield (2 steps), white solid, [M + H]⁺ = 689.4, [M + 2H]²⁺ = 345.3. ¹H NMR (400 MHz, DMSO-d6) δ ppm 1.88-2.06 (m, 5 H), 2.07- 2.27 (m, 3 H), 2.53-2.69 (m, 9 H), 3.01-3.15 (m, 7 H), 3.72 (dd, J = 11.0, 4.9 Hz, 1 H), 3.84 (s, 3 H), 4.10-4.20 (m, 1 H), 6.89 (d, J = 8.6 Hz, 2 H), 7.05 (d, J = 8.6 Hz, 2 H), 7.12 (d, J = 8.8 Hz, 2 H), 7.37 (s, 1 H), 7.59-7.65 (m, 3 H), 7.81 (d, J = 13.4 Hz, 2 H), 8.24 (s, 1 H), 9.84 (s, 1 H), 10.77 (s, 1 H). |
| TBM-38 | CBM-2 | 88 | 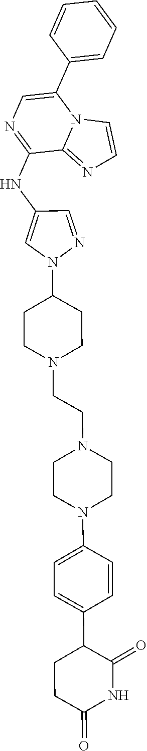 [SMILES: O=C1CCC(c2ccc(N3CCN(CCN4CCC(n5cc(Nc6ncc(-c7ccccc7)n7ccnc67)cn5)CC4)CC3)cc2)C(=O)N1] | 3-(4-(4-(2-(4-(4- ((5- phenylimidazo[1,2- a]pyrazin-8- yl)amino)-1H- pyrazol-1- yl)piperidin-1- yl)ethyl)piperazin- 1- yl)phenyl) piperidine- 2,6-dione | 97 mg, 37% yield (2 steps), white solid [M + H]⁺ = 659.4. ¹H NMR (400 MHz, DMSO-d6) δ ppm 1.88-1.95 (m, 1 H), 1.95- 2.05 (m, 3 H), 2.09-2.19 (m, 3 H), 2.41-2.47 (m, 3 H), 2.53- 2.59 (m, 4 H), 2.59-2.65 (m, 1 H), 2.96-3.04 (m, 2 H), 3.08- 3.13 (m, 3 H), 3.72 (dd, J = 10.9, 4.9 Hz, 1 H), 4.07-4.18 (m, 1 H), 6.89 (d, J = 8.4 Hz, 2 H), 7.04 (d, J = 8.6 Hz, 2 H), 7.45 (s, 1 H), 7.49-7.54 (m, 1 H), 7.54-7.60 (m, 2 H), 7.65 (d, J = 1.1 Hz, 1 H), 7.67-7.72 (m, 2 H), 7.80 (s, 1 H), 7.88 (d, J = 1.1 Hz, 1 H), 8.24 (s, 1 H), 9.90 (s, 1 H), 10.77 (s, 1 H). 4 H not observed. |
| TBM-40 | CBM-2 | 92 | 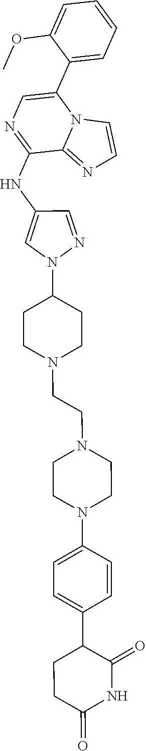 [SMILES: COc1ccccc1-c1cnc(Nc2cnn(C3CCN(CCN4CCN(c5ccc(C6CCC(=O)NC6=O)cc5)CC4)CC3)c2)c2nccn12] | 3-(4-(4-(2-(4-(4- ((5-(2- methoxyphenyl) imidazo[1,2- a]pyrazin-8- yl)amino)-1H- pyrazol-1- yl)piperidin-1- yl)ethyl)piperazin- 1- yl)phenyl) piperidine- 2,6-dione | 82 mg, 28% yield (2 steps), white solid [M+H] = 689.4. ¹H NMR (400 MHz, DMSO-d6) δ ppm 1.89-2.05 (m, 5 H), 2.09- 2.19 (m, 3 H), 2.54-2.65 (m, 6 H), 2.97-3.04 (m, 2 H), 3.09- 3.14 (m, 4 H), 3.28-3.31 (m, 4 H), 3.72 (dd, J = 11.0, 4.9 Hz, 1 H), 3.76 (s, 3 H), 4.06-4.19 (m, 1 H), 6.89 (d, J = 8.8 Hz, 2 H), 7.04 (d, J = 8.8 Hz, 2 H), 7.12 (t, J = 7.3 Hz, 1 H), 7.23 (d, J = 8.3 Hz, 1 H), 7.33-7.37 (m, 2 H), 7.43 (dd, J = 7.5, 1.8 Hz, 1 H), 7.52-7.58 (m, 2 H), 7.79 (s, 1 H), 8.23 (s, 1 H), 9.82 (s, 1 H), 10.77 (s, 1 H). |
| TBM-41 | CBM-2 | 93 | 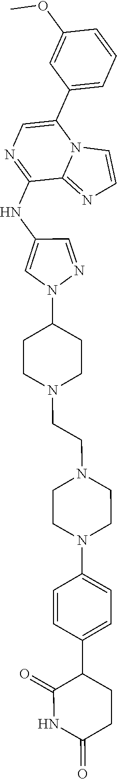 [SMILES: COc1cccc(-c2cnc(Nc3cnn(C4CCN(CCN5CCN(c6ccc(C7CCC(=O)NC7=O)cc6)CC5)CC4)c3)c3nccn23)c1] | 3-(4-(4-(2-(4-(4- ((5-(3- methoxyphenyl) imidazo[1,2- a]pyrazin-8- yl)amino)-1H- pyrazol-1- yl)piperidin-1- yl)ethyl)piperazin- 1- yl)phenyl) piperidine- 2,6-dione | 67 mg, 29% yield (2 steps), white solid [M + H]⁺ = 689.4. ¹H NMR (400 MHz, DMSO-d6) δ ppm 1.88-2.04 (m, 5 H), 2.06- 2.18 (m, 3 H), 2.53-2.64 (m, 5 H), 3.00 (br d, J = 11.4 Hz, 2 H), 3.08-3.15 (m, 4 H), 3.72 (dd, J = 10.9, 5.0 Hz, 1 H), 3.84 (s, 3 H), 4.06-4.18 (m, 1 H), 6.89 (br d, J = 8.6 Hz, 2 H), 7.01-7.10 (m, 3 H), 7.20-7.29 (m, 2 H), 7.45- 7.50 (m, 2 H), 7.64 (s, 1 H), 7.80 (s, 1 H), 7.93 (s, 1 H), 8.24 (s, 1 H), 9.90 (s, 1 H), 10.71-10.83 (m, 1 H). 5 H not observed. |
| TBM-42 | CBM-2 | 94 | 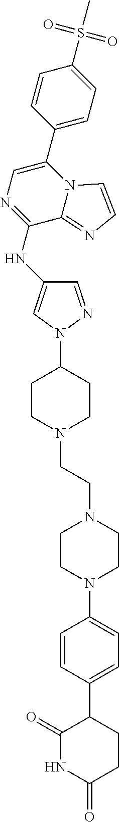 [SMILES: CS(=O)(=O)c1ccc(-c2cnc(Nc3cnn(C4CCN(CCN5CCN(c6ccc(C7CCC(=O)NC7=O)cc6)CC5)CC4)c3)c3nccn23)cc1] | 3-(4-(4-(2-(4-(4- ((5-(4- (methylsulfonyl) phenyl)imidazo[1,2- a]pyrazin-8- yl)amino)-1H- pyrazol-1- yl)piperidin-1- yl)ethyl)piperazin- 1- yl)phenyl) piperidine- 2,6-dione | 22 mg, 8% yield (2 steps), yellow solid [M + H]⁺ = 737.3. ¹H NMR (400 MHz, DMSO-d6) δ ppm 1.86-2.05 (m, 5 H), 2.07- 2.18 (m, 3 H), 2.42-2.48 (m, 2 H), 2.55-2.67 (m, 5 H), 3.01 (br d, J = 11.2 Hz, 2 H), 3.08-3.14 (m, 4 H), 3.31 (s, 3 H), 3.72 (dd, J = 10.8, 4.9 Hz, 1 H), 4.08-4.18 (m, 1 H), 6.89 (d, J = 8.8 Hz, 2 H), 7.04 (d, J = 8.8 Hz, 2 H), 7.58 (s, 1 H), 7.69 (d, J = 1.2 Hz, 1 H), 7.82 (s, 1 H), 7.97-8.03 (m, 3 H), 8.06-8.11 (m, 2 H), 8.25 (s, 1 H), 10.07 (s, 1 H), 10.77 (s, 1 H). |

### Example S23. Representative Procedure for LDD-L

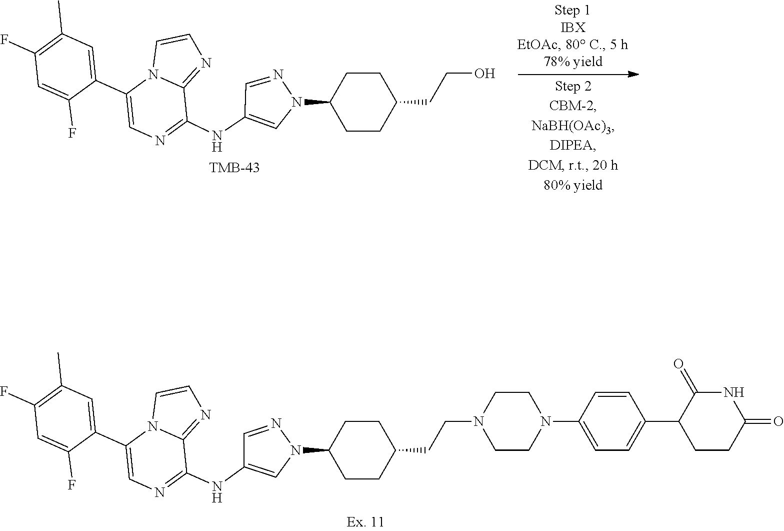

Step 1: To a suspension of 2-((trans)-4-(4-((5-(2,4-difluoro-5-methylphenyl)imidazo[1,2-a]pyrazin-8-yl)amino)-1H-pyrazol-1-yl)cyclohexyl)ethan-1-ol TBM-43 (425 mg, 0.94 mmol, 1 eq.) in ethyl acetate (10 mL) at room temperature was added IBX (526.0 mg, 1.88 mmol, 2 eq.). The reaction was then stirred at 80° C. for 5 h. The reaction was cooled to room temperature, filtered through Celite, rinsed with EtOAc and concentrated. The residue was purified by reverse phase column chromatography (5% to 100% MeCN in water). The fractions were combined and concentrated to afford 2-((trans)-4-(4-((5-(2,4-difluoro-5-methylphenyl)imidazo[1,2-a]pyrazin-8-yl)amino)-1H-pyrazol-1-yl)cyclohexyl)acetaldehyde (375 mg, 78% yield) as a tan solid.

LCMS: [M+H]⁺=451.2.

Step 2: To a solution of 2-((trans)-4-(4-((5-(2,4-difluoro-5-methylphenyl)imidazo[1,2-a]pyrazin-8-yl)amino)-1H-pyrazol-1-yl)cyclohexyl)acetaldehyde (330 mg, 0.73 mmol, 1 eq.) in DCM (7.5 mL) was added 3-(4-piperazin-1-ylphenyl)piperidine-2,6-dione CBM-2 (380 mg, 1.1 mmol, 1.5 equiv., bis HCl salt) and DIPEA (0.38 mL, 2.2 mmol, 3 eq.) at room temperature. Then NaBH(OAc)₃ (310 mg, 1.47 mmol, 3 eq.) was added and the mixture was stirred at room temperature under nitrogen atmosphere for 20 h. The DCM was removed under reduced pressure and the residue was purified by reverse phase column chromatography (500 to 100% MeCN in water w/ 0.100 formic acid). The fractions were combined, concentrated, and lyophilised to afford 3-(4-(4-(2-((trans)-4-(4-((5-(2,4-difluoro-5-methylphenyl)imidazo[1,2-a]pyrazin-8-yl)amino)-1H-pyrazol-1-yl)cyclohexyl)ethyl)piperazin-1-yl)phenyl)piperidine-2,6-dione (Compound 11) (427 mg, 80% yield) as a white solid as a full formic acid salt.

LCMS: [M+H]⁺=708.3.

¹H NMR (400 MHz, DMSO-d₆) δ ppm 1.07-1.21 (m, 2H), 1.32-1.46 (m, 3H), 1.73 (qd, J=12.3, 3.1 Hz, 2H), 1.84-1.92 (m, 2H), 1.95-2.19 (m, 4H), 2.29 (s, 3H), 2.39 (br t, J=7.2 Hz, 2H), 2.41-2.49 (m, 2H), 2.52-2.54 (m, 3H), 2.58-12.67 (m, 1H), 3.08-3.15 (m, 4H), 3.72 (dd, J=10.9, 5.0 Hz, 1H), 4.10 (tt, J=11.9, 3.8 Hz, 1H), 6.89 (d, J=8.8 Hz, 2H), 7.05 (d, J=8.8 Hz, 2H), 7.42-7.48 (m, 2H), 7.58-7.63 (m, 3H), 7.78 (s, 1H), 8.14 (s, 1H), 8.21 (s, 1H), 9.96 (s, 1H), 10.77 (s, 1H).

¹⁹F NR (377 MHz, DMSO-d₆) δ ppm −112.17 (q, J=8.2 Hz, 1 F), −111.27 (q, J=9.1 Hz, 1 F).

TABLE 38
List of Examples prepared by procedure LDD-L

| Components | | Example | | | |
|---|---|---|---|---|---|
| TBM | CBM | | | IUPAC | |
| # | # | Cmp | Structure | Name | Characterization |
| TBM-43 | CBM-2 | 11 | 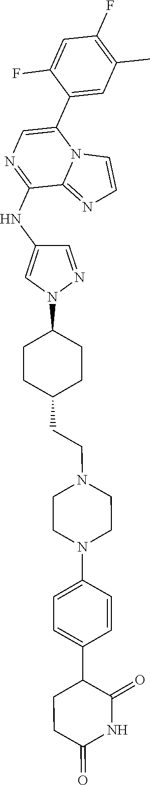 [SMILES: Cc1cc(-c2cnc(Nc3cnn([C@H]4CC[C@H](CCN5CCN(c6ccc(C7CCC(=O)NC7=O)cc6)CC5)CC4)c3)c3nccn23)c(F)cc1F] | 3-(4-(4-(2- ((trans)-4-(4- ((5-(2,4- difluoro-5- methylphenyl) imidazo[1,2- a]pyrazin-8- yl)amino)-1H- pyrazol-1- yl)cyclohexyl) ethyl)piperazin- 1- yl)phenyl) piperidine-2,6- dione | 427 mg, 62% yield (2 steps), white solid [M + H]⁺ = 708.3. ¹H NMR (400 MHz, DMSO- d6) δ ppm 1.07-1.21 (m, 2 H), 1.32-1.46 (m, 3 H), 1.73 (qd, J = 12.3, 3.1 Hz, 2 H), 1.84-1.92 (m, 2 H), 1.95- 2.19 (m, 4 H), 2.29 (s, 3 H), 2.39 (br t, J = 7.2 Hz, 2 H), 2.41-2.49 (m, 2 H), 2.52- 2.54 (m, 3 H), 2.58-2.67 (m, 1 H), 3.08-3.15 (m, 4 H), 3.72 (dd, J = 10.9, 5.0 Hz, 1 H), 4.10 (tt, J = 11.9, 3.8 Hz, 1 H), 6.89 (d, J = 8.8 Hz, 2 H), 7.05 (d, J = 8.8 Hz, 2 H), 7.42-7.48 (m, 2 H), 7.58-7.63 (m, 3 H), 7.78 (s, 1 H), 8.14 (s, 1 H), 8.21 (s, 1 H), 9.96 (s, 1 H), 10.77 (s, 1 H). ¹⁹F NMR (377 MHz, DMSO-d6) δ ppm −112.17 (q, J = 8.2 Hz, 1 F), −111.27 (q, J = 9.1 Hz, 1 F). |
| TBM-44 | CBM-2 | 14 | 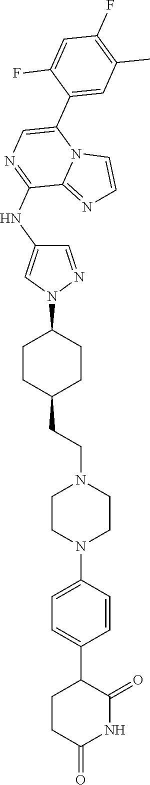 [SMILES: Cc1cc(-c2cnc(Nc3cnn([C@H]4CC[C@@H](CCN5CCN(c6ccc(C7CCC(=O)NC7=O)cc6)CC5)CC4)c3)c3nccn23)c(F)cc1F] | 3-(4-(4-(2- ((cis)-4-(4- ((5-(2,4- difluoro-5- methylphenyl) imidazo[1,2- a]pyrazin-8- yl)amino)-1H- pyrazol-1- yl)cyclohexyl) ethyl)piperazin- 1- yl)phenyl) piperidine-2,6- dione | 56 mg, 24% yield (2 steps), light pink solid [M + H]⁺ = 708.3. ¹H NMR (400 MHz, DMSO- d6) δ ppm 1.48-1.57 (m, 4 H), 1.58-1.71 (m, 3 H), 1.82 (br s, 2 H), 1.96-2.19 (m, 4 H), 2.28 (s, 3 H), 2.36 (br t, J = 7.3 Hz, 2 H), 2.41-2.48 (m, 1 H), 2.51-2.55 (m, 3 H), 2.58-2.69 (m, 1 H), 3.11 (br s, 4 H), 3.69-3.75 (m, 1 H), 4.16-4.23 (m, 1 H), 6.88 (d, J = 8.8 Hz, 2 H), 7.04 (d, J = 8.6 Hz, 2 H), 7.42-7.48 (m, 2 H), 7.58-7.64 (m, 3 H), 7.80 (s, 1 H), 8.25 (s, 1 H), 9.97 (s, 1 H), 10.77 (s, 1 H). ¹⁹F NMR (377 MHz, DMSO-d6) δ ppm −112.13 (q, J = 8.2 Hz, 1 F), −111.28 (q, J = 8.2 Hz, 1 F). |
| TBM-44 | CBM-3 | 27 | 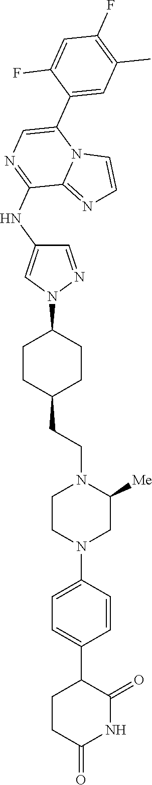 [SMILES: Cc1cc(-c2cnc(Nc3cnn([C@H]4CC[C@@H](CCN5CCN(c6ccc(C7CCC(=O)NC7=O)cc6)C[C@@H]5C)CC4)c3)c3nccn23)c(F)cc1F] | 3-(4-((S)-4- (2-((cis)-4-(4- ((5-(2,4- difluoro-5- methylphenyl) imidazo[1,2- a]pyrazin-8- yl)amino)-1H- pyrazol-1- yl)cyclohexyl) ethyl)-3- methylpiperazin- 1- yl)phenyl) piperidine-2,6- dione | 23 mg, 16% yield (2 steps), white solid [M + H]⁺ = 722.4. ¹H NMR (400 MHz, DMSO- d6) δ ppm 1.06 (br d, J = 3.4 Hz, 3 H), 1.40-1.57 (m, 4 H), 1.57-1.70 (m, 3 H), 1.73- 1.88 (m, 2 H), 1.94-2.16 (m, 4 H), 2.19-2.26 (m, 1 H), 2.28 (s, 3 H), 2.31-2.37 (m, 1 H), 2.39-2.47 (m, 1 H), 2.57-2.69 (m, 1 H), 2.71- 2.85 (m, 2 H), 2.91 (br d, J = 11.2 Hz, 1 H), 3.34-3.46 (m, 2 H), 3.71 (br dd, J = 10.9, 4.8 Hz, 1 H), 4.14- 4.26 (m, 1 H), 6.87 (br d, J = 8.1 Hz, 2 H), 7.03 (br d, J = 8.1 Hz, 2 H), 7.41-7.49 (m, 2 H), 7.57-7.66 (m, 3 H), 7.81 (s, 1 H), 8.25 (s, 1 H), 9.96 (s, 1 H), 10.76 (br s, 1 H). Two protons are not apparent in the ¹H NMR spectrum. ¹⁹F NMR (377 MHz, DMSO-d6) δ ppm −112.19- −112.07 (m, 1 F), −111.34- −111.22 (m, 1 F). |
| TBM-44 | CBM-4 | 28 | 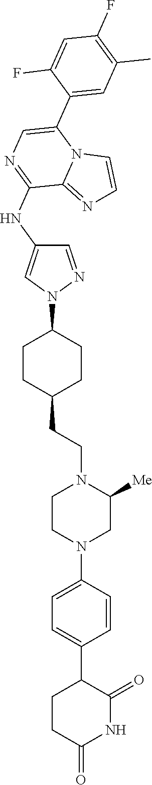 [SMILES: Cc1cc(-c2cnc(Nc3cnn([C@H]4CC[C@@H](CCN5CCN(c6ccc(C7CCC(=O)NC7=O)cc6)C[C@@H]5C)CC4)c3)c3nccn23)c(F)cc1F] | 3-(4-((R)-4- (2-((cis)-4-(4- ((5-(2,4- difluoro-5- methylphenyl) imidazo[1,2- a]pyrazin-8- yl)amino)-1H- pyrazol-1- yl)cyclohexyl) ethyl)-3- methylpiperazin- 1- yl)phenyl) piperidine-2,6- dione | 41 mg, 47% yield (2 steps), white solid [M + H]⁺ = 722.4. 1H NMR (400 MHz, DMSO-d6) δ ppm 1.06 (br d, J = 5.1 Hz, 3 H), 1.40-1.57 (m, 4 H), 1.57-1.68 (m, 3 H), 1.76-1.86 (m, 2 H), 1.95- 2.18 (m, 4 H), 2.19-2.26 (m, 1 H), 2.28 (s, 3 H), 2.30- 2.36 (m, 1 H), 2.40-2.48 (m, 2 H), 2.57-2.68 (m, 1 H), 2.70-2.82 (m, 2 H), 2.91 (br d, J = 11.2 Hz, 1 H), 3.37- 3.44 (m, 2 H), 3.71 (dd, J = 11.0, 4.9 Hz, 1 H), 4.15- 4.24 (m, 1 H), 6.87 (d, J = 8.6 Hz, 2 H), 7.03 (d, J = 8.8 Hz, 2 H), 7.41-7.48 (m, 2 H), 7.57-7.65 (m, 3 H), 7.81 (s, 1 H), 8.25 (s, 1 H), 9.96 (s, 1 H), 10.76 (s, 1 H). One proton is not apparent in the 1H NMR spectrum. ¹⁹F NMR (377 MHz, DMSO-d6) δ ppm −112.04 (br s, 1 F), −111.38-−111.21 (m, 1 F). |
| TBM-43 | CBM-4 | 37 | 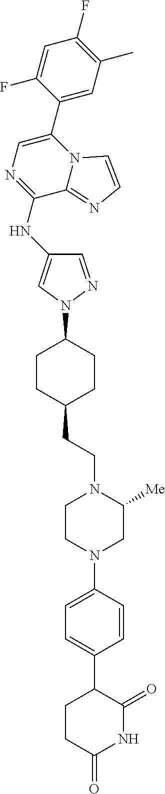 [SMILES: Cc1cc(-c2cnc(Nc3cnn([C@H]4CC[C@@H](CCN5CCN(c6ccc(C7CCC(=O)NC7=O)cc6)C[C@H]5C)CC4)c3)c3nccn23)c(F)cc1F] | 3-(4-((R)-4- (2-((trans)-4- (4-((5-(2,4- difluoro-5- methylphenyl) imidazo[1,2- a]pyrazin-8- yl)amino)-1H- pyrazol-1- yl)cyclohexyl) ethyl)-3- methylpiperazin- 1- yl)phenyl) piperidine-2,6- dione | 19 mg, 18% yield (2 steps), white solid [M + H]⁺ = 722.4. ¹H NMR (400 MHz, DMSO- d6) δ ppm 1.07 (d, J = 4.4 Hz, 3 H), 1.10-1.25 (m, 2 H), 1.33-1.44 (m, 3 H), 1.67- 1.80 (m, 2 H), 1.83-1.94 (m, 2 H), 1.95-2.19 (m, 5 H), 2.20-2.37 (m, 6 H), 2.41- 2.47 (m, 1 H), 2.58-2.69 (m, 1 H), 2.74-2.85 (m, 2 H), 2.87-2.95 (m, 1 H), 3.38- 3.46 (m, 2 H), 3.72 (dd, J = 11.0, 4.9 Hz, 1 H), 4.06- 4.16 (m, 1 H), 6.88 (d, J = 8.8 Hz, 2 H), 7.04 (d, J = 8.8 Hz, 2 H), 7.41-7.48 (m, 2 H), 7.57-7.64 (m, 3 H), 7.77 (s, 1 H), 8.21 (s, 1 H), 9.96 (s, 1 H), 10.76 (s, 1 H). ¹⁹F NMR (377 MHz, DMSO-d6) δ ppm −112.23- −112.10 (m, 1 F), −111.34- −111.20 (m, 1 F). |
| TBM-45 | CBM-2 | 40 | 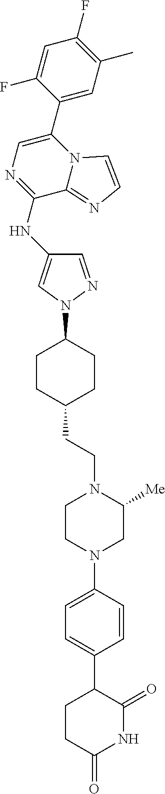 | 3-(4-(4-(2- ((trans)-4-(4- ((5-(4-methyl- 3,4-dihydro- 2H- benzo[b][1,4]] oxazin-6- yl)imidazo[1, 2-a]pyrazin-8- yl)amino)-1H- pyrazol-1- yl)cyclohexyl) ethyl)piperazin- 1- yl)phenyl) piperidine-2,6- dione | 35 mg, 54% yield (2 steps), light yellow solid [M + H]⁺ = 729.4 ¹H NMR (400 MHz, DMSO- d6) δ ppm 1.08-1.20 (m, 2 H), 1.34-1.46 (m, 3 H), 1.65- 1.78 (m, 2 H), 1.83-1.92 (m, 2 H), 1.94-2.21 (m, 5 H), 2.37 (br t, J = 7.1 Hz, 2 H), 2.42-2.48 (m, 2 H), 2.58- 2.68 (m, 1 H), 2.89 (s, 3 H), 3.09-3.14 (m, 4 H), 3.72 (dd, J = 11.0, 4.9 Hz, 1 H), 4.03-4.15 (m, 1 H), 4.27- 4.32 (m, 2 H), 6.81-6.92 (m, 5 H), 7.05 (d, J = 8.6 Hz, 2 H), 7.37 (s, 1 H), 7.61 (d, J = 1.0 Hz, 1 H), 7.76 (s, 1 H), 7.88 (d, J = 1.2 Hz, 1 H), 8.21 (s, 1 H), 9.77 (s, 1 H), 10.77 (s, 1 H). |
| TBM-46 | CBM-2 | 45 | 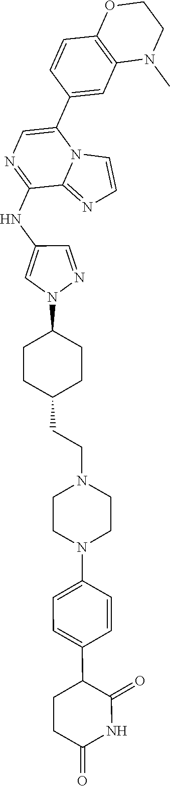 | 3-(4-(4-(2- ((cis)-4-(4- ((5-(m- tolyl)imidazo [1,2-a]pyrazin- 8-yl)amino)- 1H-pyrazol-1- yl)cyclohexyl) ethyl)piperazin- 1- yl)phenyl) piperidine-2,6- dione | 97 mg, 32% yield (2 steps), tan solid [M + H]⁺ = 672.4. ¹H NMR (400 MHz, DMSO- d6) δ ppm 1.47-1.57 (m, 4 H), 1.57-1.72 (m, 3 H), 1.75- 1.87 (m, 2 H), 1.95-2.19 (m, 4 H), 2.31-2.45 (m, 6 H), 2.45-2.49 (m, 1 H), 2.52- 2.56 (m, 3 H), 2.58-2.68 (m, 1 H), 3.07-3.15 (m, 4 H), 3.72 (dd, J = 10.8, 4.9 Hz, 1 H), 4.15-4.24 (m, 1 H), 6.88 (d, J = 8.8 Hz, 2 H), 7.04 (d, J = 8.8 Hz, 2 H), 7.32 (d, J = 7.1 Hz, 1 H), 7.42-7.51 (m, 4 H), 7.64 (d, J = 1.2 Hz, 1 H), 7.80 (s, 1 H), 7.89 (d, J = 1.2 Hz, 1 H), 8.26 (s, 1 H), 9.87 (s, 1 H), 10.77 (s, 1 H). |
| TBM-47 | CBM-2 | 47 | 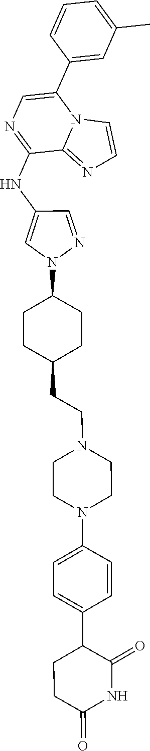 | 3-(4-(4-(2- ((trans)-4-(4- ((5- cyclopropyl imidazo[1,2- a]pyrazin-8- yl)amino)-1H- pyrazol-1- yl)cyclohexyl) ethyl)piperazin- 1- yl)phenyl) piperidine-2,6- dione | 16 mg, 16% yield (2 steps), white solid [M + H]⁺ = 622.3; [M + 2H]²⁺ = 311.8 ¹H NMR (400 MHz, DMSO- d6) δ ppm 0.69-0.76 (m, 2 H), 0.96-1.03 (m, 2 H), 1.06- 1.20 (m, 2 H), 1.33-1.47 (m, 3 H), 1.70 (qd, J = 12.5, 2.8 Hz, 2 H), 1.87 (br d, J = 12.5 Hz, 2 H), 1.95-2.10 (m, 5 H), 2.10-2.19 (m, 1 H), 2.34-2.41 (m, 2 H), 2.41- 2.48 (m, 2 H), 2.57-2.66 (m, 1 H), 3.08-3.15 (m, 4 H), 3.73 (dd, J = 11.0, 4.9 Hz, 1 H), 4.02-4.12 (m, 1 H), 6.89 (d, J = 8.8 Hz, 2 H), 7.05 (d, J = 8.8 Hz, 2 H), 7.21 (d, J = 1.2 Hz, 1 H), 7.65 (d, J = 1.0 Hz, 1 H), 7.71 (s, 1 H), 8.05 (d, J = 0.7 Hz, 1 H), 8.15 (s, 1 H), 9.63 (s, 1 H), 10.77 (s, 1 H). |
| TBM-43 | CBM-3 | 57 | 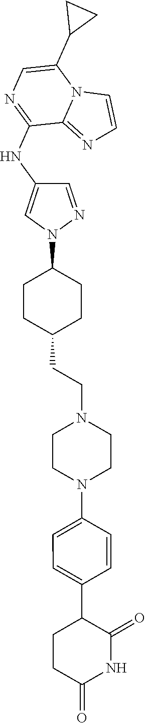 [SMILES: O=C1CCC(c2ccc(N3CCN(CC[C@H]4CC[C@H](n5cc(Nc6ncc(C7CC7)n7ccnc67)cn5)CC4)CC3)cc2)C(=O)N1] | 3-(4-((S)-4- (2-((trans)-4- (4-((5-(2,4- difluoro-5- methylphenyl) imidazo[1,2- a]pyrazin-8- yl)amino)-1H- pyrazol-1- yl)cyclohexyl) ethyl)-3- methylpiperazin- 1- yl)phenyl) piperidine-2,6- dione | 11 mg, 10% yield (2 steps), tan solid [M + H]⁺ = 722.3. ¹H NMR (400 MHz, DMSO- d6) δ ppm 1.07 (d, J = 5.4 Hz, 3 H), 1.10-1.25 (m, 2 H), 1.31-1.45 (m, 3 H), 1.65- 1.80 (m, 2 H), 1.82-1.94 (m, 2 H), 1.96-2.18 (m, 4 H), 2.20-2.31 (m, 5 H), 2.42- 2.48 (m, 3 H), 2.58-2.68 (m, 1 H), 2.73-2.84 (m, 2 H), 2.86-2.93 (m, 1 H), 3.38- 3.45 (m, 2 H), 3.72 (dd, J = 10.9, 5.0 Hz, 1 H), 4.05- 4.16 (m, 1 H), 6.88 (d, J = 8.8 Hz, 2 H), 7.04 (d, J = 8.8 Hz, 2 H), 7.41-7.48 (m, 2 H), 7.57-7.64 (m, 3 H), 7.76- 7.79 (m, 1 H), 8.20-8.21 (m, 1 H), 8.21-8.23 (m, 1 H), 9.96 (s, 1 H), 10.76 (s, 1 H). ¹⁹F NMR (377 MHz, DMSO-d6) δ ppm −112.16 (g, J = 8.2 Hz, 1 F), −111.27 (q, J = 9.5 Hz, 1 F). |
| TBM-48 | CBM-2 | 64 | 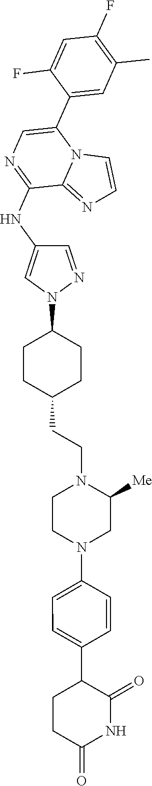 | 3-(4-(4-(2- ((trans)-4-(4- ((5-(m- tolyl)imidazo [[1,2-a]pyrazin- 8-yl)amino)- 1H-pyrazol-1- yl)cyclohexyl) ethyl)piperazin- 1- yl)phenyl) piperidine-2,6- dione | 21 mg, 19% yield (2 steps), off-white solid [M + H]⁺ = 672.3 ¹H NMR (400 MHz, DMSO- d6) δ ppm 1.08-1.26 (m, 3 H), 1.32-1.48 (m, 3 H), 1.66- 1.79 (m, 2 H), 1.88 (br d, J = 13.2 Hz, 2 H), 1.96-2.19 (m, 4 H), 2.41 (s, 3 H), 2.42- 2.48 (m, 3 H), 2.54-2.69 (m, 4 H), 3.08-3.18 (m, 4 H), 3.73 (dd, J = 11.0, 4.9 Hz, 1 H), 4.10 (tt, J = 11.8, 3.8 Hz, 1 H), 6.90 (d, J = 8.6 Hz, 2 H), 7.05 (d, J = 8.8 Hz, 2 H), 7.30-7.34 (m, 1 H), 7.41-7.51 (m, 4 H), 7.64 (d, J = 1.0 Hz, 1 H), 7.77 (s, 1 H), 7.89 (d, J = 1.2 Hz, 1 H), 8.21 (s, 1 H), 9.87 (s, 1 H), 10.77 (s, 1 H). |
| TBM-51 | CBM-2 | 66 | 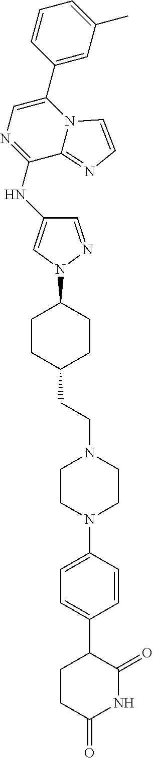 | 3-(4-(4-(2- ((trans)-3-(4- ((5-(2,4- difluoro-5- methylphenyl) imidazo[1,2- a]pyrazin-8- yl)amino)-1H- pyrazol-1- yl)cyclobutyl) ethyl)piperazin- 1- yl)phenyl) piperidine-2,6- dione | 16 mg, 6% yield (2 steps), white solid [M + H]⁺ = 680.3., [M + 2H]²⁺ = 340.7. ¹H NMR (400 MHz, DMSO- d6) δ ppm 1.75 (q, J = 6.6 Hz, 2 H), 1.95-2.05 (m, 1 H), 2.08-2.22 (m, 3 H), 2.25- 2.38 (m, 7 H), 2.40-2.48 (m, 1 H), 2.52-2.65 (m, 6 H), 3.12 (br s, 4 H), 3.72 (dd, J = 11.0, 5.1 Hz, 1 H), 4.92- 5.02 (m, 1 H), 6.89 (d, J = 8.6 Hz, 2 H), 7.05 (d, J = 8.6 Hz, 2 H), 7.41-7.49 (m, 2 H), 7.58-7.65 (m, 3 H), 7.83 (s, 1 H), 8.24 (s, 1 H), 9.99 (s, 1 H), 10.77 (s, 1 H). ¹⁹F NMR (377 MHz, DMSO-d6) δ ppm −112.14 (q, J = 8.2 Hz, 1 F), −111.25 (q, J = 8.2 Hz, 1 F). |
| TBM-52 | CBM-2 | 67 | 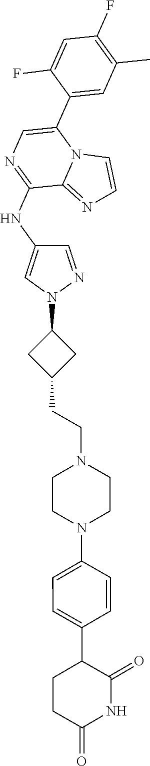 | 3-(4-(4- (((trans)-4-(4- ((5-(2,4- difluoro-5- methylphenyl) imidazo[1,2- a]pyrazin-8- yl)amino)-1H- pyrazol-1- yl)cyclohexyl) methyl)piperazin-1- yl)phenyl) piperidine-2,6- dione | 20 mg, 9% yield (2 steps), white solid [M + H]⁺ = 694.4; [M + 2H]²⁺ = 347.8. ¹H NMR (400 MHz, DMSO- d6) δ ppm 0.75-0.90 (m, 1 H), 1.02-1.17 (m, 2 H), 1.58- 1.69 (m, 1 H), 1.69-1.83 (m, 2 H), 1.89-1.97 (m, 2 H), 1.98-2.09 (m, 3 H), 2.10- 2.16 (m, 1 H), 2.19 (d, J = 7.0 Hz, 2 H), 2.29 (s, 3 H), 2.40-2.47 (m, 2 H), 2.52- 2.56 (m, 2 H), 2.57-2.65 (m, 1 H), 3.09-3.16 (m, 4 H), 3.72 (dd, J = 11.1, 4.8 Hz, 1 H), 4.06-4.17 (m, 1 H), 6.89 (d, J = 8.8 Hz, 2 H), 7.05 (d, J = 8.7 Hz, 2 H), 7.39-7.50 (m, 2 H), 7.56- 7.66 (m, 3 H), 7.78 (s, 1 H), 8.21 (s, 1 H), 9.97 (s, 1 H), 10.77 (s, 1 H). ¹⁹F NMR (377 MHz, DMSO-d6) δ ppm −112.18 (q, J = 9.1 Hz, 1 F), −111.27 (q, J = 8.2 Hz, 1 F). |
| TBM-53 | CBM-2 | 71 | 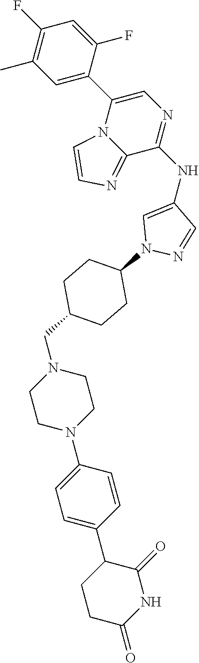 [SMILES: Cc1cc(-c2cnc(Nc3cnn([C@H]4CC[C@H](CN5CCN(c6ccc(C7CCC(=O)NC7=O)cc6)CC5)CC4)c3)c3nccn23)c(F)cc1F] | 3-(4-(4- (((cis)-4-(4- ((5-(2,4- difluoro-5- methylphenyl) imidazo[1,2- a]pyrazin-8- yl)amino)-1H- pyrazol-1- yl)cyclohexyl) methyl)piperazin-1- yl)phenyl) piperidine-2,6- dione | 15 mg, 2% yield (2 steps), white solid [M + H]⁺ = 694.4. ¹H NMR (400 MHz, DMSO- d6) δ ppm 1.51-1.69 (m, 4 H), 1.78-1.93 (m, 3 H), 1.95- 2.07 (m, 3 H), 2.10-2.20 (m, 1 H), 2.26-2.31 (m, 5 H), 2.40-2.48 (m, 2 H), 2.58- 2.65 (m, 1 H), 3.11 (br s, 4 H), 3.72 (dd, J = 10.9, 4.8 Hz, 1 H), 4.18-4.26 (m, 1 H), 6.88 (d, J = 8.6 Hz, 2 H), 7.04 (d, J = 8.6 Hz, 2 H), 7.42-7.48 (m, 2 H), 7.58- 7.65 (m, 3 H), 7.80 (s, 1 H), 8.26 (s, 1 H), 9.96 (s, 1 H), 10.76 (s, 1 H). ¹⁹F NMR (377 MHz, DMSO-d6) δ ppm −112.13 (q, J = 8.2 Hz, 1 F), −111.29 (q, J = 8.6 Hz, 1 F). |
| TBM-43 | CBM-23 | 81 | 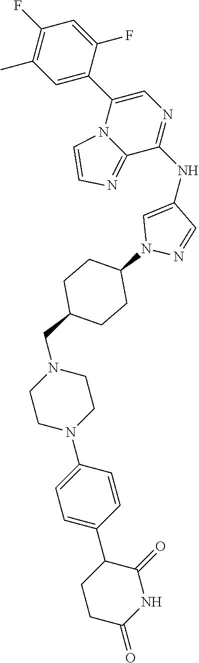 | (R)-3-(4-(4- (2-((trans)-4- (4-((5-(2,4- difluoro-5- methylphenyl) imidazo[1,2- a]pyrazin-8- yl)amino)-1H- pyrazol-1- yl)cyclohexyl) ethyl)piperazin- 1- yl)phenyl)-3- methylpiperidine- 2,6-dione | 65 mg, 58% yield (2 steps), white solid [M + H]⁺ = 777.3, 779.3. ¹H NMR (400 MHz, DMSO- d6 ) δ ppm 1.07 (d, J = 5.6 Hz, 3 H), 1.78 (d, J = 6.6 Hz, 3 H), 1.84-1.95 (m, 3 H), 1.97-2.03 (m, 4 H), 2.05- 2.17 (m, 3 H), 2.34-2.47 (m, 5 H), 2.59-2.68 (m, 1 H), 2.72-2.88 (m, 2 H), 2.90- 3.03 (m, 3 H), 3.37-3.45 (m, 3 H), 3.72 (dd, J = 11.1, 4.8 Hz, 1 H), 3.93-4.07 (m, 1 H), 5.63-5.70 (m, 2 H), 6.00 (q, J = 6.5 Hz, 1 H), 6.62 (d, J = 1.5 Hz, 1 H), 6.88 (d, J = 8.6 Hz, 2 H), 7.04 (d, J = 8.8 Hz, 2 H), 7.45 (t, J = 1.0 Hz, 1 H), 7.53- 7.59 (m, 2 H), 7.70 (s, 1 H), 10.77 (s, 1 H). ¹⁹F NMR (377 MHz, DMSO-d6) δ ppm −113.37- −113.28 (m, 1 F). |
| TBM-43 | CBM-22 | 82 | 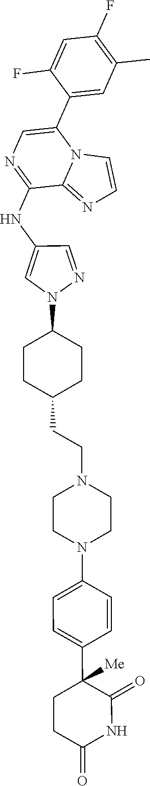 [SMILES: Cc1cc(-c2cnc(Nc3cnn([C@H]4CC[C@H](CCN5CCN(c6ccc([C@@]7(C)CCC(=O)NC7=O)cc6)CC5)CC4)c3)c3nccn23)c(F)cc1F] | (S)-3-(4-(4- (2-((trans)-4- (4-((5-(2,4- difluoro-5- methylphenyl) imidazo[1,2- a]pyrazin-8- yl)amino)-1H- pyrazol-1- yl)cyclohexyl) ethyl)piperazin- 1- yl)phenyl)-3- methylpiperidine- 2,6-dione | 52 mg, 47% yield (2 steps), white solid [M + H]⁺ = 722.3. ¹H NMR (400 MHz, DMSO- d6 ) δ ppm 1.09-1.19 (m, 2 H), 1.36-1.44 (m, 6 H), 1.67- 1.78 (m, 2 H), 1.84-1.91 (m, 2 H), 2.00-2.10 (m, 4 H), 2.27-2.33 (m, 4 H), 2.34- 2.45 (m, 4 H), 3.09-3.16 (m, 4 H), 4.06-4.15 (m, 1 H), 6.91 (br d, J = 8.8 Hz, 2 H), 7.10 (d, J = 8.8 Hz, 2 H), 7.42-7.48 (m, 2 H), 7.58- 7.63 (m, 3 H), 7.78 (s, 1 H), 8.21 (s, 1 H), 9.97 (s, 1 H), 10.84 (s, 1 H) ¹⁹F NMR (377 MHz, DMSO-d6) δ ppm −112.20- −112.11 (m, 1 F), −111.34- −111.21 (m, 1 F). |
| TBM-49 | CBM-2 | 91 | 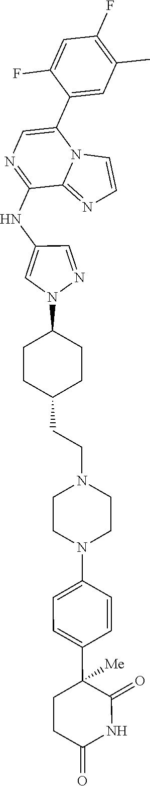 | 3-(4-(4-(2- ((trans)-4-(4- ((5-(7-fluoro- 3,4-dihydro- 2H- benzo[b][1,4]] oxazin-6- yl)imidazo[1, 2-a]pyrazin-8- yl)amino)-1H- pyrazol-1- yl)cyclohexyl) ethyl)piperazin- 1- yl)phenyl) piperidine-2,6- dione | Product was treated with 4M HCl (diox), r.t., 20 h, to remove the boc-group: 14 mg, 16% yield (3 steps), light-yellow solid [M + H]⁺ = 733.4. ¹H NMR (400 MHz, DMSO- d6) δ ppm 1.15-1.25 (m, 3 H), 1.37-1.47 (m, 2 H), 1.64- 1.71 (m, 2 H), 1.74-1.82 (m, 2 H), 1.85-1.91 (m, 2 H), 1.98-2.02 (m, 1 H), 2.05- 2.10 (m, 2 H), 2.13-2.18 (m, 1 H), 2.43-2.46 (m, 1 H), 2.61-2.68 (m, 1 H), 3.03- 3.15 (m, 4 H), 3.17-3.23 (m, 2 H), 3.30-3.34 (m, 2 H), 3.56-3.61 (m, 2 H), 3.77- 3.83 (m, 2 H), 4.22-4.24 (m, 2 H), 6.73 (d, J = 7.1 Hz, 1 H), 6.80 (d, J = 11.0 Hz, 1 H), 6.97 (br d, J = 8.8 Hz, 2 H), 7.12 (br d, J = 8.6 Hz, 2 H), 7.37 (s, 1 H), 7.66 (br s, 1 H), 7.73-7.77 (m, 2 H), 8.20 (s, 1 H), 10.15-10.36 (m, 3 H), 10.79 (s, 1 H). ¹⁹F NMR (377 MHz, DMSO-d6) δ ppm −125.61 (s, 1 F). |
| TBM-43 | CBM-24 | 95 | 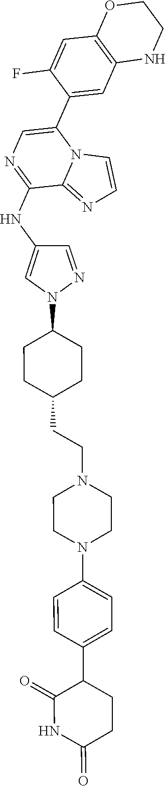 [SMILES: O=C1CCC(c2ccc(N3CCN(CC[C@H]4CC[C@H](n5cc(Nc6ncc(-c7cc8c(cc7F)OCCN8)n7ccnc67)cn5)CC4)CC3)cc2)C(=O)N1] | 3-(4-(1-(2- ((trans)-4-(4- ((5-(2,4- difluoro-5- methylphenyl) imidazo[1,2- a]pyrazin-8- yl)amino)-1H- pyrazol-1- yl)cyclohexyl) ethyl)piperidin-4- yl)phenyl) piperidine-2,6- dione | 13 mg, 11% yield (2 steps), white solid [M + H]⁺ = 707.4, [M + 2H]²⁺ = 354.3. ¹H NMR (400 MHz, DMSO- d6) δ ppm 1.06-1.20 (m, 2 H), 1.34-1.47 (m, 3 H), 1.62- 1.79 (m, 6 H), 1.83-1.92 (m, 2 H), 1.98-2.08 (m, 5 H), 2.10-2.23 (m, 1 H), 2.29 (s, 3 H), 2.36-2.42 (m, 2 H), 2.44-2.48 (m, 2 H), 2.60- 2.65 (m, 1 H), 3.01 (br d, J = 9.9 Hz, 2 H), 3.81 (br dd, J = 11.2, 4.8 Hz, 1 H), 4.05- 4.16 (m, 1 H), 7.13 (d, J = 8.2 Hz, 2 H), 7.21 (d, J = 7.9 Hz, 2 H), 7.39-7.50 (m, 2 H), 7.57-7.64 (m, 3 H), 7.78 (s, 1 H), 8.21 (s, 1 H), 9.97 (s, 1 H), 10.81 (s, 1 H). ¹⁹F NMR (377 MHz, DMSO-d6) δ ppm −112.17 (br d, J = 8.2 Hz, 1 F), −111.26 (br d, J = 8.2 Hz, 1 F). |
| TBM-56 | CBM-2 | 100 | 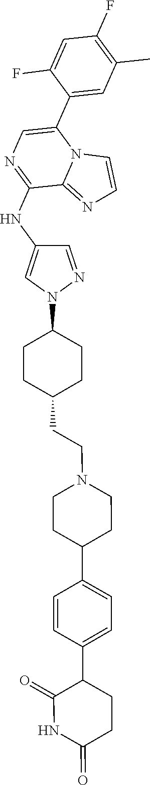 | 3-(4-(4-(2- ((trans)-4-(4- ((5- (bicyclo[3.1.0]] hexan-1- yl)imidazo[1, 2-a]pyrazin-8- yl)amino)-1H- pyrazol-1- yl)cyclohexyl) ethyl)piperazin- 1- yl)phenyl) piperidine-2,6- dione | 16 mg, 8% yield (2 steps), white solid [M + H]⁺= 662.4. ¹H NMR (400 MHz, DMSO- d6 ) δ ppm 0.76-0.82 (m, 1 H), 0.98-1.02 (m, 1 H), 1.11- 1.18 (m, 2 H), 1.36-1.46 (m, 4 H), 1.64-1.77 (m, 5 H), 1.82-1.91 (m, 4 H), 2.01- 2.20 (m, 7 H), 2.31-2.40 (m, 3 H), 2.60-2.69 (m, 2 H), 2.87-2.95 (m, 1 H), 3.09- 3.15 (m, 4 H), 3.70-3.75 (m, 1 H), 4.04-4.11 (m, 1 H), 6.89 (br d, J = 8.8 Hz, 2 H), 7.05 (br d, J = 8.3 Hz, 2 H), 7.31 (s, 1 H), 7.64 (s, 1 H), 7.72 (s, 1 H), 7.85 (s, 1 H), 8.14 (s, 1 H), 9.64 (s, 1 H), 10.77 (s, 1 H). |
| TBM-44 | CBM-25 | 101 | 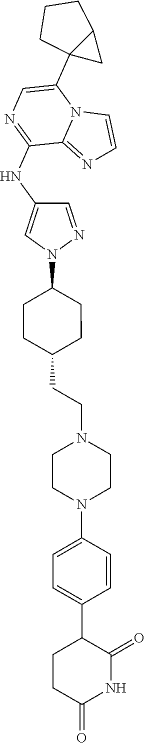 [SMILES: O=C1CCC(c2ccc(N3CCN(CC[C@H]4CC[C@H](n5cc(Nc6ncc(C78CCCC7C8)n7ccnc67)cn5)CC4)CC3)cc2)C(=O)N1] | 3-(4-(6-(2- ((cis)-4-(4- ((5-(2,4- difluoro-5- methylphenyl) imidazo[1,2- a]pyrazin-8- yl)amino)-1H- pyrazol-1- yl)cyclohexyl) ethyl)-2,6- diazaspiro[3.3]] heptan-2- yl)phenyl) piperidine-2,6- dione | 29 mg, 14% yield (2 steps), off-white solid [M + H]⁺ = 720.3, [M + 2H]²⁺ = 360.7. ¹H NMR (400 MHz, DMSO- d6) δ ppm 1.26-1.36 (m, 2 H), 1.41-1.51 (m, 2 H), 1.54- 1.66 (m, 3 H), 1.75-1.84 (m, 2 H), 1.93-2.15 (m, 5 H), 2.29 (br s, 3 H), 2.36- 2.44 (m, 3 H), 2.55-2.66 (m, 2 H), 3.69 (dd, J = 10.9, 5.0 Hz, 1 H), 3.82-3.85 (m, 4 H), 4.15-4.23 (m, 1 H), 6.37 (d, J = 8.6 Hz, 2 H), 6.99 (d, J = 8.6 Hz, 2 H), 7.42-7.47 (m, 2 H), 7.58- 7.63 (m, 3 H), 7.80 (s, 1 H), 8.18 (s, 1 H), 8.24 (br s, 1 H), 9.96 (s, 1 H), 10.74 (s, 1 H) ¹⁹F NMR (377 MHz, DMSO-d6) δ ppm −112.13 (d, J = 8.2 Hz, 1 F), −111.27 (d, J = 8.2 Hz, 1 F). |
| TBM-50 | CBM-2 | 103 | 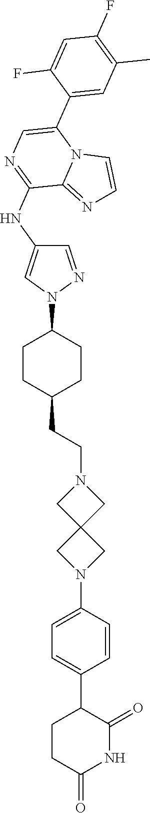 | 3-(4-(4-(2- ((cis)-4-(4- ((5-(2,4- difluoro-5- methylphenyl) imidazo[1,2- alpyridin-8- yl)amino)-1H- pyrazol-1- yl)cyclohexyl) ethyl)piperazin- 1- yl)phenyl) piperidine-2,6- dione | 23 mg, 40% yield (2 steps), off-white solid [M + H]⁺ = 707.3; [M + 2H]²⁺ = 354.2 ¹H NMR (400 MHz, DMSO- d6) δ ppm 1.48-1.72 (m, 7 H), 1.76-1.87 (m, 2 H), 1.96- 2.21 (m, 4 H), 2.28 (s, 3 H), 2.30-2.38 (m, 2 H), 2.40- 2.47 (m, 1 H), 2.56-2.69 (m, 2 H), 3.07-3.15 (m, 4 H), 3.72 (dd, J = 11.0, 4.9 Hz, 1 H), 4.12-4.24 (m, 1 H), 6.53 (d, J = 7.8 Hz, 1 H), 6.75 (d, J = 7.6 Hz, 1 H), 6.88 (d, J = 8.6 Hz, 2 H), 7.04 (d, J = 8.6 Hz, 2 H), 7.41 (t, J = 10.0 Hz, 1 H), 7.46 (d, J = 2.0 Hz, 1 H), 7.50-7.56 (m, 3 H), 7.94 (s, 1 H), 8.11 (s, 1 H), 10.76 (s, 1 H). ¹⁹F NMR (377 MHz, DMSO-d6) δ ppm −112.50- −112.23 (m, 1 F), −111.98- −111.82 (m, 1 F). |
| TBM-43 | CBM-25 | 104 | 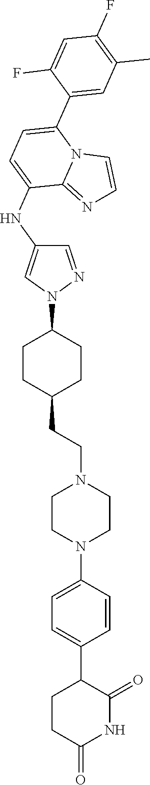 | 3-(4-(6-(2- ((trans)-4-(4- ((5-(2,4- difluoro-5- methylphenyl) imidazo[1,2- a]pyrazin-8- yl)amino)-1H- pyrazol-1- yl)cyclohexyl) ethyl)-2,6- diazaspiro[3.3]] heptan-2- yl)phenyl) piperidine-2,6- dione | 29 mg, 9% yield (2 steps), white solid [M + H]⁺ = 720.3, [M + 2H]²⁺ = 360.8. ¹H NMR (400 MHz, DMSO- d6) δ ppm 1.02-1.25 (m, 5 H), 1.35 (br s, 1 H), 1.63- 1.84 (m, 4 H), 1.92-2.15 (m, 5 H), 2.29 (s, 3 H), 2.36- 2.45 (m, 3 H), 2.55-2.67 (m, 2 H), 3.70 (dd, J = 10.8, 4.9 Hz, 1 H), 3.84 (s, 4 H), 4.03-4.14 (m, 1 H), 6.38 (d, J = 8.6 Hz, 2 H), 7.00 (d, J = 8.6 Hz, 2 H), 7.42-7.48 (m, 2 H), 7.58-7.65 (m, 3 H), 7.78 (s, 1 H), 8.20 (s, 1 H), 9.96 (s, 1 H), 10.74 (s, 1 H). ¹⁹F NMR (377 MHz, DMSO-d6) δ ppm −112.12 (d, J = 8.2 Hz, 1 F), −111.22 (d, J = 8.2 Hz, 1 F) |
| TBM-43 | CBM-10 | 105 | 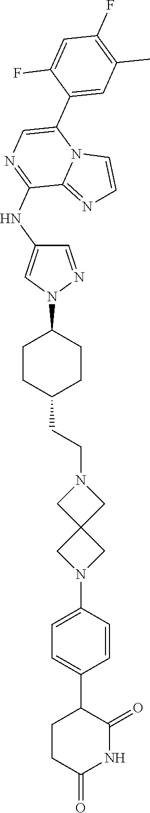 | 3-(6-(4-(2- ((trans)-4-(4- ((5-(2,4- difluoro-5- methylphenyl) imidazo[1,2- a]pyrazin-8- yl)amino)-1H- pyrazol-1- yl)cyclohexyl) ethyl)piperazin- 1-yl)-1- methyl-1H- indazol-3- yl)piperidine- 2,6-dione | 57 mg, 25% yield (2 steps), off-white solid [M + H]⁺ = 762.4, [M + 2H]²⁺ = 381.8 ¹H NMR (400 MHz, DMSO- d6) δ ppm 1.08-1.22 (m, 2 H), 1.35-1.48 (m, 3 H), 1.67- 1.80 (m, 2 H), 1.84-1.93 (m, 2 H), 2.00-2.20 (m, 3 H), 2.28 (br s, 4 H), 2.35- 2.44 (m, 2 H), 2.52-2.57 (m, 4 H), 2.58-2.65 (m, 2 H), 3.19-3.26 (m, 4 H), 3.89 (s, 3 H), 4.06-4.16 (m, 1 H), 4.23 (dd, J = 9.3, 5.1 Hz, 1 H), 6.84 (s, 1 H), 6.93 (dd, J = 9.0, 1.0 Hz, 1 H), 7.42- 7.52 (m, 3 H), 7.58-7.65 (m, 3 H), 7.78 (s, 1 H), 8.21 (s, 1 H), 9.97 (s, 1 H), 10.85 (s, 1 H). ¹⁹F NMR (377 MHz, DMSO-d6) δ ppm −112.17 (d, J = 8.2 Hz, 1 F), −111.22 (d, J = 8.2 Hz, 1 F). |
| TBM-43 | CBM-27 | 106 | 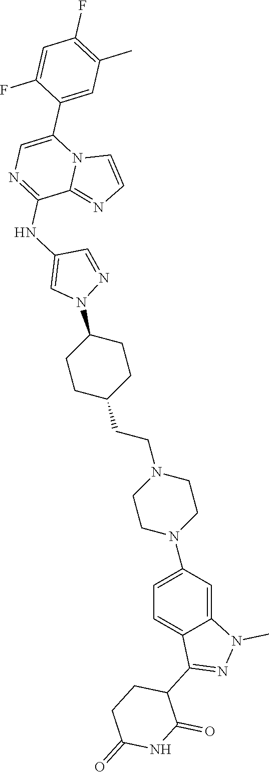 [SMILES: Cc1cc(-c2cnc(Nc3cnn([C@H]4CC[C@H](CCN5CCN(c6ccc7c(C8CCC(=O)NC8=O)nn(C)c7c6)CC5)CC4)c3)c3nccn23)c(F)cc1F] | 1-(6-(4-(2- ((trans)-4-(4- ((5-(2,4- difluoro-5- methylphenyl) imidazo[1,2- a]pyrazin-8- yl)amino)-1H- pyrazol-1- yl)cyclohexyl) ethyl)piperazin- 1-yl)-1- methyl-1H- indazol-3- yl)dihydropyri midine- 2,4(1H,3H)- dione | 43 mg, 37% yield (2 steps), tan solid [M + H]⁺ = 763.4 ¹H NMR (400 MHz, DMSO- d6) δ ppm 1.07-1.22 (m, 2 H), 1.35-1.50 (m, 3 H), 1.66- 1.81 (m, 2 H), 1.84-1.94 (m, 2 H), 2.00-2.10 (m, 2 H), 2.29 (s, 3 H), 2.35-2.44 (m, 3 H), 2.52-2.58 (m, 4 H), 2.73 (t, J = 6.6 Hz, 2 H), 3.24 (br s, 4 H), 3.89-3.92 (m, 2 H), 4.05-4.16 (m, 1 H), 6.82 (d, J = 1.5 Hz, 1 H), 6.92 (dd, J = 9.2, 1.8 Hz, 1 H), 7.41-7.50 (m, 3 H), 7.58- 7.65 (m, 3 H), 7.78 (s, 1 H), 8.21 (s, 1 H), 9.97 (s, 1 H), 10.50 (s, 1 H). ¹⁹F NMR (377 MHz, DMSO-d6) δ ppm −112.17 (q, J = 8.2 Hz, 1 F), −111.27 (q, J = 8.6 Hz, 1 F). |
| TBM-43 | CBM-28 | 107 | 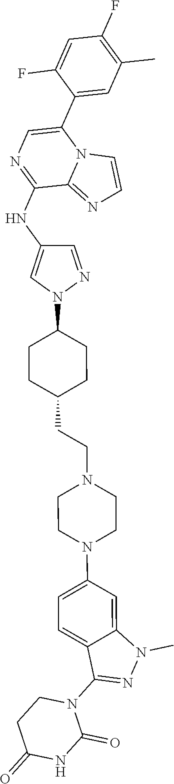 [SMILES: Cc1cc(-c2cnc(Nc3cnn([C@H]4CC[C@H](CCN5CCN(c6ccc7c(N8CCC(=O)NC8=O)nn(C)c7c6)CC5)CC4)c3)c3nccn23)c(F)cc1F] | 1-(7-(4-(2- ((trans)-4-(4- ((5-(2,4- difluoro-5- methylphenyl) imidazo[1,2- a]pyrazin-8- yl)amino)-1H- pyrazol-1- yl)cyclohexyl) ethyl)piperazin- 1-yl)-1- methyl-1H- indazol-3- yl) dihydropyrimidine- 2,4(1H,3H)- dione | 27 mg, 23% yield (2 steps), tan solid M + H]⁺ = 763.4 ¹H NMR (400 MHz, DMSO- d6) δ ppm 1.09-1.22 (m, 2 H), 1.43 (br dd, J = 7.0, 5.5 Hz, 3 H), 1.73 (br dd, J = 12.5, 2.7 Hz, 2 H), 1.85- 1.94 (m, 2 H), 2.01-2.08 (m, 2 H), 2.29 (s, 3 H), 2.36 (s, 3 H), 2.39-2.44 (m, 2 H), 2.52-2.63 (m, 4 H), 2.79 (t, J = 6.7 Hz, 2 H), 3.26 (br s, 4 H), 3.65-3.79 (m, 2 H), 4.05- 4.17 (m, 1 H), 6.74 (d, J = 7.3 Hz, 1 H), 7.02 (dd, J = 7.8, 0.7 Hz, 1 H), 7.07-7.14 (m, 1 H), 7.41-7.48 (m, 2 H), 7.57-7.65 (m, 3 H), 7.76- 7.81 (m, 1 H), 8.21 (s, 1 H), 9.97 (s, 1 H), 10.45 (s, 1 H). ¹⁹F NMR (377 MHz, DMSO-d6) δ ppm −112.17 (q, J = 8.6 Hz, 1 F), −111.27 (q, J = 9.1 Hz, 1 F). |
| TBM-59 | CBM-2 | 108 | 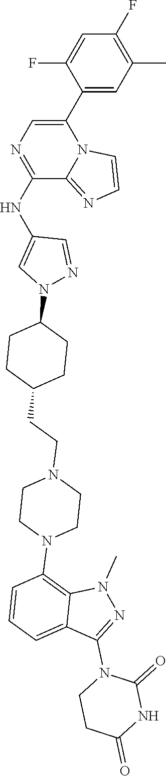 [SMILES: Cc1cc(-c2cnc(Nc3cnn([C@H]4CC[C@H](CCN5CCN(c6cccc7c(N8CCC(=O)NC8=O)nn(C)c67)CC5)CC4)c3)c3nccn23)c(F)cc1F] | 3-(4-(4-(2- ((trans)-4-(4- ((5-(2,4- difluoro-5- methylphenyl) imidazo[1,2- alpyridin-8- yl)amino)-1H- pyrazol-1- yl)cyclohexyl) ethyl)piperazin- 1- yl)phenyl) piperidine-2,6- dione | 9 mg, 10% yield (2 steps), off-white solid [M + H]⁺ = 707.3, [M + 2H]²⁺ = 354.2. ¹H NMR (400 MHz, DMSO- d6) δ ppm 1.07-1.20 (m, 2 H), 1.34-1.47 (m, 3 H), 1.70- 1.82 (m, 2 H), 1.88 (br d, J = 12.0 Hz, 2 H), 1.95-2.18 (m, 3 H), 2.28 (s, 3 H), 2.31- 2.47 (m, 5 H), 2.57-2.70 (m, 1 H), 3.11 (br s, 4 H), 3.72 (dd, J = 11.0, 4.9 Hz, 1 H), 4.03-4.15 (m, 1 H), 6.51 (d, J = 7.6 Hz, 1 H), 6.74 (d, J = 7.6 Hz, 1 H), 6.89 (d, J = 8.8 Hz, 2 H), 7.05 (d, J = 8.6 Hz, 2 H), 7.41 (t, J = 10.0 Hz, 1 H), 7.45 (d, J = 2.0 Hz, 1 H), 7.49-7.56 (m, 3 H), 7.88 (s, 1 H), 8.10 (s, 1 H), 10.77 (s, 1 H). ¹⁹F NMR (377 MHz, DMSO-d6) δ ppm −112.44- −112.24 (m, 1 F), −111.96- −111.81 (m, 1 F). |
| TBM-43 | CBM-11 | 117 | 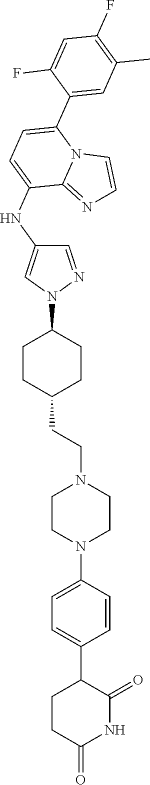 | 3-(7-(4-(2- ((trans)-4-(4- ((5-(2,4- difluoro-5- methylphenyl) imidazo[1,2- a]pyrazin-8- yl)amino)-1H- pyrazol-1- yl)cyclohexyl) ethyl)piperazin- 1-yl)-1- methyl-1H- indazol-3- yl)piperidine- 2,6-dione | 54.8 mg, 29% yield (2 steps), white solid [M + H]⁺ = 762.4 1H NMR (400 MHz, DMSO-d6) δ ppm 1.06- 1.25 (m, 2 H), 1.35-1.50 (m, 3 H), 1.67-1.81 (m, 2 H), 1.84-1.93 (m, 2 H), 2.00- 2.09 (m, 2 H), 2.11-2.21 (m, 1 H), 2.29 (s, 4 H), 2.58- 2.66 (m, 2 H), 2.78-3.00 (m, 4 H), 3.04-3.20 (m, 2 H), 4.06-4.16 (m, 1 H), 4.24 (s, 3 H), 4.30 (dd, J = 9.8, 5.4 Hz, 1 H), 6.99-7.07 (m, 2 H), 7.37 (dd, J = 7.3, 2.0 Hz, 1 H), 7.42-7.49 (m, 2 H), 7.58-7.65 (m, 3 H), 7.78 (s, 1 H), 8.21 (s, 1 H), 9.98 (s, 1 H), 10.90 (s, 1 H). ¹⁹F NMR (377 MHz, DMSO-d6) δ ppm −112.07 (q, J = 8.2 Hz, 1 F), −111.23 (q, J = 8.2 Hz, 1 F). |
| TBM-57 | CBM-2 | 118 | 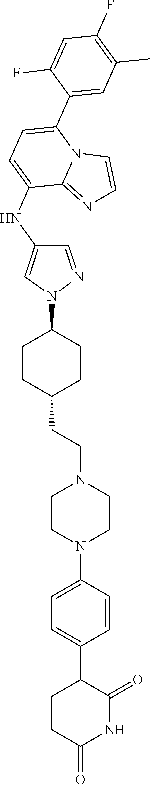 | 3-(4-(4-(2- ((trans)-4-(4- ((5-(4,5,6,7- tetrahydropyrazolo [1,5- alpyridin-3- yl)imidazo[1, 2-a]pyrazin-8- yl)amino)-1H- pyrazol-1- yl)cyclohexyl) ethyl)piperazin- 1- yl)phenyl) piperidine-2,6- dione | 18 mg, 12% yield (2 steps), tan solid [M + H]⁺ = 702.4, [M + 2H]²⁺ = 351.7. ¹H NMR (400 MHz, DMSO- d6) δ ppm 1.31-1.51 (m, 4 H), 1.63-1.93 (m, 8 H), 1.95- 2.19 (m, 7 H), 2.34-2.39 (m, 2 H), 2.71-2.79 (m, 2 H), 3.06-3.17 (m, 5 H), 3.72 (br dd, J = 11.0, 4.9 Hz, 1 H), 4.02-4.22 (m, 4 H), 6.85 (br d, J = 8.8 Hz, 2 H), 7.00 (br d, J = 8.8 Hz, 2 H), 7.30 (s, 1 H), 7.59 (d, J = 1.0 Hz, 1 H), 7.72-7.84 (m, 3 H), 8.19 (s, 1 H), 9.75 (s, 1 H), 10.77 (s, 1 H). |
| TBM-58 | CBM-2 | 120 | 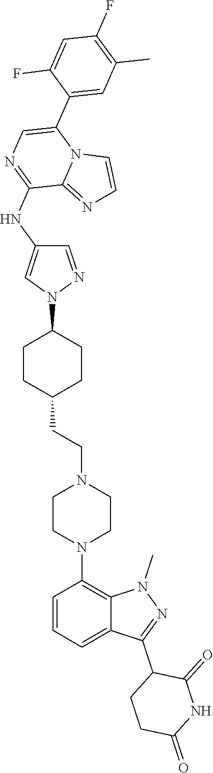 | 3-(4-(4-(2- ((trans)-4-(4- ((5-(1H- pyrazol-4- yl)imidazo[1, 2-a]pyrazin-8- yl)amino)-1H- pyrazol-1- yl)cyclohexyl) ethyl)piperazin- 1- yl)phenyl) piperidine-2,6- dione | 11 mg, 32% yield (2 steps), off-white solid [M + H]⁺ = 648.4 ¹H NMR (400 MHz, DMSO- d6) δ ppm 1.09-1.22 (m, 2 H), 1.33-1.51 (m, 3 H), 1.64- 1.81 (m, 2 H), 1.88 (br d, J = 12.5 Hz, 2 H), 1.96- 2.22 (m, 5 H), 2.37 (br t, J = 7.0 Hz, 2 H), 2.44 (br s, 1 H), 2.58-2.65 (m, 1 H), 3.12 (br s, 5 H), 3.50 (br d, J = 7.8 Hz, 1 H), 3.72 (dd, J = 11.1, 4.8 Hz, 1 H), 4.03- 4.15 (m, 1 H), 6.89 (d, J = 8.6 Hz, 2 H), 7.05 (d, J = 8.6 Hz, 2 H), 7.56 (s, 1 H), 7.67 (s, 1 H), 7.75 (s, 1 H), 8.05 (s, 2 H), 8.18- 8.21 (m, 1 H), 8.21-8.39 (m, 1 H), 9.76 (s, 1 H), 10.77 (s, 1 H), 13.11-13.44 (m, 1 H). One 1H not apparent. |

### Example S24. Representative Procedure for LDD-M

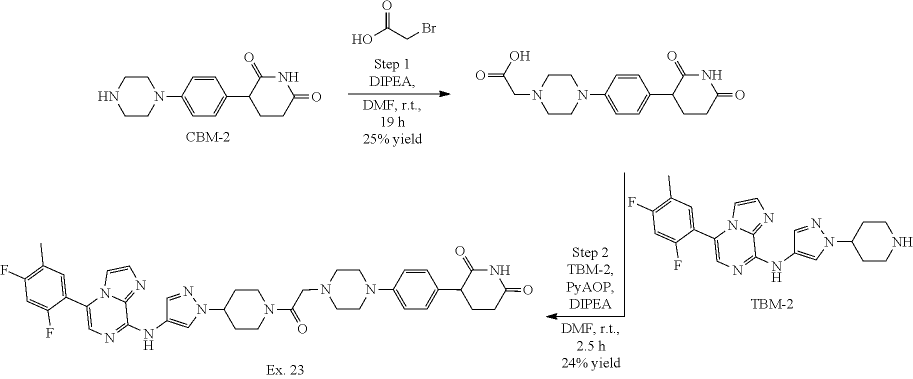 [SMILES: O=C(O)CBr ; O=C1CCC(c2ccc(N3CCNCC3)cc2)C(=O)N1 ; O=C(O)CN1CCN(c2ccc(C3CCC(=O)NC3=O)cc2)CC1 ; Cc1cc(-c2cnc(Nc3cnn(C4CCN(C(=O)CN5CCN(c6ccc(C7CCC(=O)NC7=O)cc6)CC5)CC4)c3)c3nccn23)c(F)cc1F ; Cc1cc(-c2cnc(Nc3cnn(C4CCNCC4)c3)c3nccn23)c(F)cc1F]

Step 1: To a solution of 3-(4-piperazin-1-ylphenyl)piperidine-2,6-dione hydrochloride CBM-2 (200 mg, 0.65 mmol, 1 eq.) in DMF (0.80 mL) were added 2-bromoacetic acid (99 mg, 0.71 mmol, 1.1 eq.) and DIPEA (0.56 mL, 3.23 mmol, 5 eq.) at room temperature. The mixture was stirred at room temperature for 19 h. The solution was then directly purified by reverse phase column chromatography (5% to 100% MeCN in water w/0.1% formic acid). The fractions were combined and concentrated, affording 2-(4-(4-(2,6-dioxopiperidin-3-yl)phenyl)piperazin-1-yl)acetic acid (45 mg, 21% yield) as a yellow solid. ¹H NMR showed around 15% w/w of the bis alkylated product.

LCMS: [M+H]⁺=332.2.

¹H NMR (400 MHz, DMSO-d₆) δ ppm 1.95-2.05 (m, 1H), 2.06-2.20 (m, 1H), 2.41-2.47 (m, 1H), 2.58-2.67 (m, 1H), 2.68-2.75 (m, 4H), 3.12-3.17 (m, 4H), 3.21 (s, 2H), 3.73 (dd, J=11.0, 4.9 Hz, 1H), 6.89 (d, J=8.6 Hz, 2H), 7.05 (d, J=8.6 Hz, 2H), 10.77 (s, 1H).

Step 2: To a solution of 2-(4-(4-(2,6-dioxopiperidin-3-yl)phenyl)piperazin-1-yl)acetic acid (45 mg, 0.14 mmol, 1 eq.) in DMF (0.68 mL) at room temperature were added DIPEA (0.24 mL, 1.36 mmol, 10 eq.) and 5-(2,4-difluoro-5-methylphenyl)-N-(1-(piperidin-4-yl)-1H-pyrazol-4-yl)imidazo[1,2-a]pyrazin-8-amine dihydrochloride TBM-2 (92 mg, 0.19 mmol, 1.4 eq.). The resulting solution was stirred at room temperature for 10 minutes. Then, PyAOP (92 mg, 0.18 mmol, 1.3 eq.) was added in one portion. The reaction mixture was stirred at room temperature for 2.5 h. The crude mixture was purified by reverse phase column chromatography (5% to 100% MeCN in water w/ 0.1% formic acid) The fractions were combined, concentrated, and repurified by prep-LCMS to give 3-(4-(4-(2-(4-(4-((5-(2,4-difluoro-5-methylphenyl)imidazo[1,2-a]pyrazin-8-yl)amino)-1H-pyrazol-1-yl)piperidin-1-yl)-2-oxoethyl)piperazin-1-yl)phenyl)piperidine-2,6-dione (Compound 23) (25 mg, 24% yield) as a white solid.

LCMS: [M+H]⁺=723.4.

¹H NMR (400 MHz, DMSO-d₆) δ ppm 1.64-1.78 (m, 1H), 1.89-2.17 (m, 5H), 2.29 (s, 3H), 2.40-2.48 (m, 1H), 2.54-2.66 (m, 5H), 2.70-2.81 (m, 1H), 3.11-3.22 (m, 6H), 3.34-3.41 (m, 1H), 3.70 (dd, J=11.0, 4.9 Hz, 1H), 4.20 (br d, J=12.7 Hz, 1H), 4.41-4.51 (m, 2H), 6.89 (d, J=8.8 Hz, 2H), 7.01 (d, J=8.6 Hz, 2H), 7.42-7.48 (m, 2H), 7.58-7.63 (m, 3H), 7.80 (s, 1H), 8.24 (s, 1H), 10.00 (s, 1H), 10.76 (s, 1H).

¹⁹F NMR (377 MHz, DMSO-d₆) δ ppm −112.23-−112.05 (m, 1 F), −111.29-−111.14 (m, 1 F).

TABLE 39
List of Examples prepared by procedure LDD-M

| Components | | Example | | | |
|---|---|---|---|---|---|
| TBM# | CBM# | Cmp | Structure | IUPAC Name | Characterization |
| TBM-2 | CBM-2 | 23 | 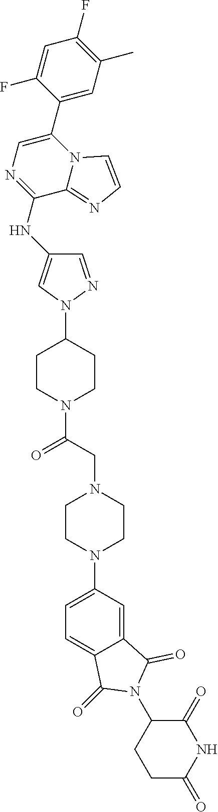 | 3-(4-(4-(2-(4-(4-((5-(2,4- difluoro-5- methylphenyl)imidazo[1, 2-a]pyrazin-8-yl)amino)- 1H-pyrazol-1- yl)piperidin-1-y1)-2- oxoethyl)piperazin-1- yl)phenyl)piperidine-2,6- dione | 25 mg, 5% yield (2 steps), white solid [M + H]⁺ = 723.4 ¹H NMR (400 MHz, DMSO-d6) δ ppm 1.64-1.78 (m, 1 H), 1.89-2.17 (m, 5 H), 2.29 (s, 3 H), 2.40- 2.48 (m, 1 H), 2.54- 2.66 (m, 5 H), 2.70- 2.81 (m, 1 H), 3.11- 3.22 (m, 6 H), 3.34- 3.41 (m, 1 H), 3.70 (dd, J = 11.0, 4.9 Hz, 1 H), 4.20 (br d, J = 12.7 Hz, 1 H), 4.41- 4.51 (m, 2 H), 6.89 (d, J = 8.8 Hz, 2 H), 7.01 (d, J = 8.6 Hz, 2 H), 7.42-7.48 (m, 2 H), 7.58-7.63 (m, 3 H), 7.80 (s, 1 H), 8.24 (s, 1 H), 10.00 (s, 1 H), 10.76 (s, 1 H). ¹⁹F NMR (377 MHz, DMSO-d6) δ ppm −112.23-−112.05 (m, 1 F), −111.29- −111.14 (m, 1 F). |
| TBM-2 | CBM-1 | 24 | 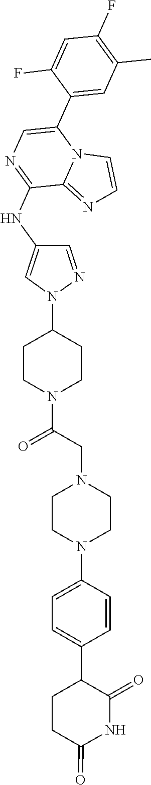 | 5-(4-(2-(4-(4-((5-(2,4- difluoro-5- methylphenyl)imidazo[1, 2-a]pyrazin-8-yl)amino)- 1H-pyrazol-1- yl)piperidin-1-yl)-2- oxoethyl)piperazin-1-y1)- 2-(2,6-dioxopiperidin-3- yl)isoindoline-1,3-dione | 44 mg, 8% yield (2 steps), yellow solid [M + H]⁺ = 792.3 ¹H NMR (400 MHz, DMSO-d6) δ ppm 1.66-1.80 (m, 1 H), 1.91-2.11 (m, 4 H), 2.28 (s, 3 H), 2.55- 2.55 (m, 1 H), 2.57- 2.63 (m, 5 H), 2.77 (br t, J = 13.0 Hz, 1 H), 2.82-2.95 (m, 1 H), 3.13-3.24 (m, 2 H), 3.34-3.41 (m, 1 H), 3.48 (br s, 4 H), 4.19 (br d, J = 12.2 Hz, 1 H), 4.40-4.52 (m, 2 H), 5.07 (dd, J = 12.8, 5.3 Hz, 1 H), 7.26 (br d, J = 8.6 Hz, 1 H), 7.36 (s, 1 H), 7.39-7.47 (m, 2 H), 7.56-7.67 (m, 4 H), 7.81 (s, 1 H), 8.25 (s, 1 H), 10.00 (s, 1 H), 11.07 (s, 1 H). ¹⁹F NMR (377 MHz, DMSO-d6) δ ppm −112.19 (s, 1 F), −111.19 (s, 1 F). |

### Example S25. Representative Procedure for LDD-N

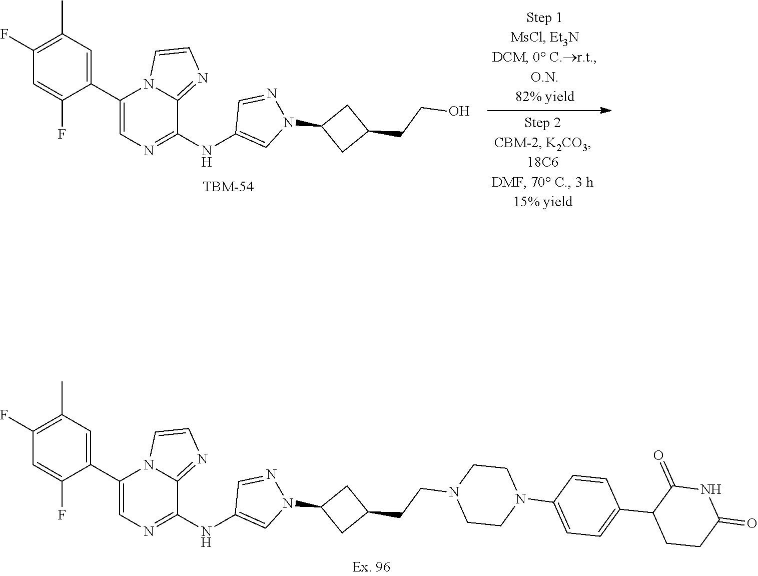 [SMILES: Cc1cc(-c2cnc(Nc3cnn([C@H]4C[C@@H](CCO)C4)c3)c3nccn23)c(F)cc1F ; Cc1cc(-c2cnc(Nc3cnn([C@H]4C[C@@H](CCN5CCN(c6ccc(C7CCC(=O)NC7=O)cc6)CC5)C4)c3)c3nccn23)c(F)cc1F]

Step 1: In a flask under N₂ at 0° C., 2-((cis)-3-(4-((5-(2,4-difluoro-5-methylphenyl)imidazo[1,2-a]pyrazin-8-yl)amino)-1H-pyrazol-1-yl)cyclobutyl)ethan-1-ol TBM-54 (43.0 mg, 0.10 mmol) was dissolved in DCM (3 mL). Then NEt₃ (0.04 mL, 0.30 mmol) was added, followed by MsCl (0.02 mL, 0.30 mmol). The reaction was stirred at room temperature, overnight. The reaction was quenched with water and extracted with DCM. The organic layers were combined, dried over MgSO₄, filtered, and concentrated. The residue was purified by reverse phase column chromatography (5% to 100% MeCN in water w/ 0.100 formic acid). The fractions were collected and concentrated, affording 2-((cis)-3-(4-((5-(2,4-difluoro-5-methylphenyl)imidazo[1,2-a]pyrazin-8-yl)amino)-1H-pyrazol-1-yl)cyclobutyl)ethyl methanesulfonate (42 mg, 82% yield) as a light-orange semi-solid.

LCMS: [M+H]⁺=503.2.

¹H NMR (400 MHz, DMSO-d₆) δ ppm 1.85-1.93 (m, 2H), 2.12-2.22 (m, 3H), 2.30 (s, 3H), 2.54-2.61 (m, 2H), 3.19 (s, 3H), 4.20 (t, J=6.4 Hz, 2H), 4.66-4.79 (m, 1H), 7.42-7.50 (m, 2H), 7.59-7.66 (m, 3H), 7.80-7.86 (m, 1H), 8.22-8.26 (m, 1H), 9.96-10.03 (m, 1H).

Step 2: In a flask under N₂ atmosphere were dissolved 2-((cis)-3-(4-((5-(2,4-difluoro-5-methylphenyl)imidazo[1,2-a]pyrazin-8-yl)amino)-1H-pyrazol-1-yl)cyclobutyl)ethyl methanesulfonate (42 mg, 0.08 mmol), 3-(4-piperazin-1-ylphenyl)piperidine-2,6-dione CBM-2 (39 mg, 0.13 mmol), 18-Crown-6 (11 mg, 0.04 mmol) and K₂CO₃ (24 mg, 0.17 mmol) in DMF (2 mL). The reaction was then stirred at 70° C. for 3 hours, LCMS showed 15% conversion. The reaction was stopped, concentrated, and purified by reverse phase column chromatography (5% to 50% MeCN in 0.02 M aqueous HCl). The fractions were collected and further purified by prep-HPLC (C18, using MeCN/HCl 0.02 M as eluent), affording 3-(4-(4-(2-((cis)-3-(4-((5-(2,4-difluoro-5-methylphenyl)imidazo[1,2-a]pyrazin-8-yl)amino)-1H-pyrazol-1-yl)cyclobutyl)ethyl)piperazin-1-yl)phenyl)piperidine-2,6-dione (Compound 96) (8.6 mg, 15% yield) as a light yellow semi-solid.

LCMS: [M+H]⁺=680.4, [M+2H]²⁺=340.7.

¹H NMR (400 MHz, DMSO-d₆) δ ppm 1.91-2.12 (m, 5H), 2.12-2.23 (m, 3H), 2.26-2.36 (m, 4H), 2.55-2.70 (m, 4H), 3.04-3.19 (m, 6H), 3.54-3.61 (m, 3H), 4.70-4.80 (m, 1H), 6.97 (d, J=8.8 Hz, 2H), 7.11 (d, J=8.6 Hz, 2H), 7.45-7.50 (m, 2H), 7.60 (t, J=8.3 Hz, 1H), 7.72 (br s, 1H), 7.76 (s, 1H), 7.82 (s, 1H), 8.23 (s, 1H), 10.33-10.47 (m, 2H), 10.80 (s, 1H).

TABLE 40
List of Examples prepared by procedure LDD-N

| Components | | Example | | | |
|---|---|---|---|---|---|
| TBM# | CBM# | Cmp | Structure | IUPAC Name | Characterization |
| TBM-54 | CBM-2 | 96 | 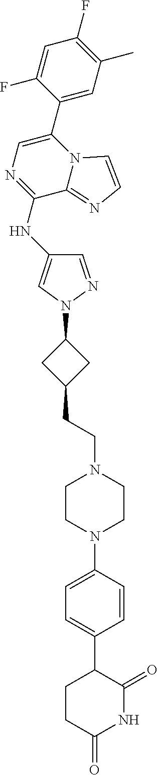 [SMILES: Cc1cc(-c2cnc(Nc3cnn([C@H]4C[C@@H](CCN5CCN(c6ccc(C7CCC(=O)NC7=O)cc6)CC5)C4)c3)c3nccn23)c(F)cc1F] | 3-(4-(4-(2-((cis)-3-(4-((5-(2,4- difluoro-5- methylphenyl)imidazo[1,2- alpyrazin-8-y1)amino)-1H- pyrazol-1- yl)cyclobutyl)ethyl)piperazin- 1-yl)phenyl)piperidine-2,6- dione | 8.6 mg, 12% yield (2 steps), light yellow semi-solid [M + H]⁺ = 680.4, [M + 2H]²⁺ = 340.7. ¹H NMR (400 MHz, DMSO-d6) δ ppm 1.91-2.12 (m, 5 H), 2.12- 2.23 (m, 3 H), 2.26-2.36 (m, 4 H), 2.55-2.70 (m, 4 H), 3.04- 3.19 (m, 6 H), 3.54-3.61 (m, 3 H), 4.70-4.80 (m, 1 H), 6.97 (d, J = 8.8 Hz, 2 H), 7.11 (d, J = 8.6 Hz, 2 H), 7.45- 7.50 (m, 2 H), 7.60 (t, J = 8.3 Hz, 1 H), 7.72 (br s, 1 H), 7.76 (s, 1 H), 7.82 (s, 1 H), 8.23 (s, 1 H), 10.33-10.47 (m, 2 H), 10.80 (s, 1 H). |
| TBM-55 | CBM-2 | 99 | 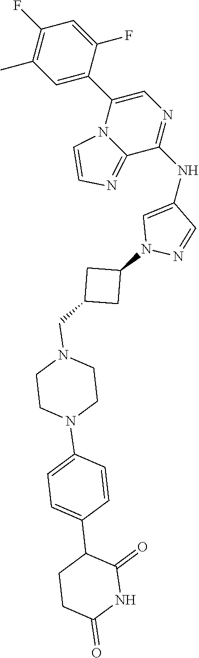 [SMILES: Cc1cc(-c2cnc(Nc3cnn([C@H]4C[C@H](CN5CCN(c6ccc(C7CCC(=O)NC7=O)cc6)CC5)C4)c3)c3nccn23)c(F)cc1F] | 3-(4-(4-(((trans)-3-(4-((5-(2,4- difluoro-5- methylphenyl)imidazo[1,2- alpyrazin-8-yl)amino)-1H- pyrazol-1- yl)cyclobutyl)methyl)piperazin- 1-yl)phenyl)piperidine-2,6- dione | 8.5 mg, 7% yield (2 steps), yellow semi-solid [M + H]⁺ = 666.3, [M + 2H]²⁺ = 333.7 ¹H NMR (400 MHz, DMSO-d6) δ ppm 1.96-2.04 (m, 1 H), 2.08- 2.21 (m, 1 H), 2.29 (s, 3 H), 2.46 (m, 2 H), 2.60- 2.74 (m, 4 H), 2.89-2.98 (m, 1 H), 3.05-3.12 (m, 2 H), 3.13- 3.24 (m, 3 H), 3.53 (m, 2 H), 3.73-3.85 (m, 4 H), 4.94-5.02 (m, 1 H), 6.97 (d, J = 8.8 Hz, 2 H), 7.12 (d, J = 8.8 Hz, 2 H), 7.42- 7.50 (m, 2 H), 7.61 (t, J = 8.6 Hz, 1 H), 7.66 (s, 2 H), 7.87 (s, 1 H), 8.27 (s, 1 H), 9.94-10.15 (m, 2 H), 10.79 (s, 1 H). |

### Example S26. Representative Procedure for LDD-O

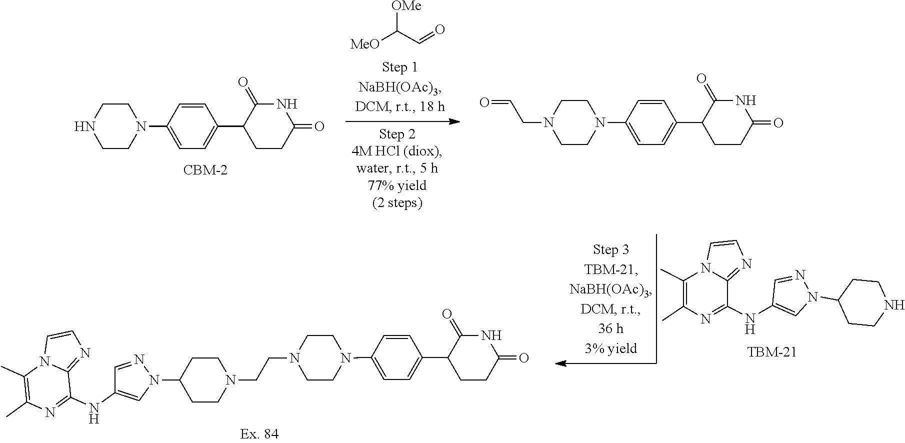 [SMILES: COC(C=O)OC ; O=C1CCC(c2ccc(N3CCNCC3)cc2)C(=O)N1 ; O=CCN1CCN(c2ccc(C3CCC(=O)NC3=O)cc2)CC1 ; Cc1nc(Nc2cnn(C3CCN(CCN4CCN(c5ccc(C6CCC(=O)NC6=O)cc5)CC4)CC3)c2)c2nccn2c1C ; Cc1nc(Nc2cnn(C3CCNCC3)c2)c2nccn2c1C]

Step 1: To a solution of 3-(4-piperazin-1-ylphenyl)piperidine-2,6-dione dihydrochloride CBM-2 (500 mg, 1.44 mmol) in DCM (14.4 mL) was added 2,2-dimethoxyacetaldehyde 60% w/w in H₂O (0.33 mL, 2.17 mmol) and NaBH(OAc)₃ (0.61 g, 2.89 mmol). The reaction mixture was stirred at room temperature for 18 h. The suspension was then concentrated to dryness and purified by reverse phase column chromatography (5% to 100% MeCN in water w/ 0.1% formic acid). The fractions were collected and concentrated, affording 3-(4-(4-(2,2-dimethoxyethyl)piperazin-1-yl)phenyl)piperidine-2,6-dione (408 mg, 73% yield) as a tan solid.

LCMS: [M+H]⁺=362.2.

¹H NMR (400 MHz, DMSO-d₆) δ ppm 1.92-2.06 (m, 1H), 2.06-2.21 (m, 1H), 2.42-2.49 (m, 3H), 2.55-2.60 (m, 4H), 2.60-2.70 (m, 1H), 3.04-3.14 (m, 4H), 3.27 (s, 6H), 3.72 (dd, J=11.0, 4.9 Hz, 1H), 4.52 (t, J=5.1 Hz, 1H), 6.88 (d, J=8.8 Hz, 2H), 7.05 (d, J=8.8 Hz, 2H), 10.77 (s, 1H).

Step 2: To a flask charged with 3-(4-(4-(2,2-dimethoxyethyl)piperazin-1-yl)phenyl)piperidine-2,6-dione (147 mg, 0.410 mmol) was added 4.0 M HCl in 1,4-dioxane (3.05 mL, 12.2 mmol) followed by water (0.050 mL). The reaction was stirred at room temperature for 5 h. The mixture was then concentrated to dryness, chased with MeCN (3×) and dried in vacuo affording 2-(4-(4-(2,6-dioxopiperidin-3-yl)phenyl)piperazin-1-yl)acetaldehydedihydrochloride (189 mg, 99% yield) as a tan solid.

LCMS: [M+H]⁺=316.2

Step 3: To a suspension of 5,6-dimethyl-N-(1-(piperidin-4-yl)-1H-pyrazol-4-yl)imidazo[1,2-a]pyrazin-8-amine TBM-21 (95 mg, 0.31 mmol) and 2-(4-(4-(2,6-dioxopiperidin-3-yl)phenyl)piperazin-1-yl)acetaldehyde dihydrochloride (181 mg, 0.40 mmol) in DCM (4.07 mL) was added DIPEA (0.21 mL, 1.22 mmol). The mixture was stirred for 10 min, then NaBH(OAc)₃ (84 mg, 0.40 mmol) was added and the stirring continued for 18 h, after which additional aldehyde (95 mg, 0.310 mmol) and NaBH(OAc)₃ (84. mg, 0.40 mmol) were added. After another 18 h, the reaction mixture was concentrated and purified by reverse phase column chromatography (5% to 100% MeCN in water w/ 0.1% formic acid) followed by preparative HPLC, affording 3-(4-(4-(2-(4-(4-((5,6-dimethylimidazo[1,2-a]pyrazin-8-yl)amino)-1H-pyrazol-1-yl)piperidin-1-yl)ethyl)piperazin-1-yl)phenyl)piperidine-2,6-dione (Compound 84) (6.9 mg, 3% yield) as an off-white solid.

LCMS: [M+H]⁺=611.4.

¹H NMR (400 MHz, DMSO-d₆) δ ppm 1.86-2.04 (m, 5H), 2.07-2.18 (m, 3H), 2.37 (s, 3H), 2.44 (s, 3H), 2.53-2.58 (m, 4H), 2.58-2.65 (m, 1H), 3.00 (br d, J=12.2 Hz, 2H), 3.09-3.13 (m, 4H), 3.72 (dd, J=11.0, 4.9 Hz, 1H), 4.04-4.17 (m, 1H), 6.89 (d, J=8.8 Hz, 2H), 7.05 (d, J=8.6 Hz, 2H), 7.58 (d, J=1.0 Hz, 1H), 7.78 (s, 1H), 7.84 (d, J=1.0 Hz, 1H), 8.22 (s, 1H), 8.43 (s, 2H), 9.52 (s, 1H), 10.77 (s, 1H), 7H not apparent.

TABLE 41
List of Examples prepared by procedure LDD-O

| Components | | Example | | | |
|---|---|---|---|---|---|
| TBM# | CBM# | Cmp | Structure | IUPAC Name | Characterization |
| TBM-21 | CBM-2 | 84 | 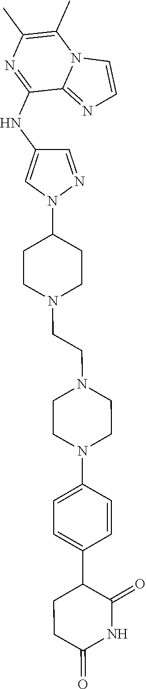 [SMILES: Cc1nc(Nc2cnn(C3CCN(CCN4CCN(c5ccc(C6CCC(=O)NC6=O)cc5)CC4)CC3)c2)c2nccn2c1C] | 3-(4-(4-(2-(4-(4-((5,6- dimethylimidazo[1,2- a]pyrazin-8-yl)amino)-1H- pyrazol-1-yl)piperidin-1- yl)ethyl)piperazin-1- yl)phenyl)piperidine-2,6- dione | 6.9 mg, 2% yield (3 steps), off-white solid [M + H]⁺ = 611.4 ¹H NMR (400 MHz, DMSO-d6) δ ppm 1.86-2.04 (m, 5 H), 2.07- 2.18 (m, 3 H), 2.37 (s, 3 H), 2.44 (s, 3 H), 2.53-2.58 (m, 4 H), 2.58-2.65 (m, 1 H), 3.00 (br d, J = 12.2 Hz, 2 H), 3.09-3.13 (m, 4 H), 3.72 (dd, J = 11.0, 4.9 Hz, 1 H), 4.04-4.17 (m, 1 H), 6.89 (d, J = 8.8 Hz, 2 H), 7.05 (d, J = 8.6 Hz, 2 H), 7.58 (d, J = 1.0 Hz, 1 H), 7.78 (s, 1 H), 7.84 (d, J = 1.0 Hz, 1 H), 8.22 (s, 1 H), 8.43 (s, 2 H), 9.52 (s, 1 H), 10.77 (s, 1H), 7 H not apparent. |
| TBM-39 | CBM-2 | 89 | 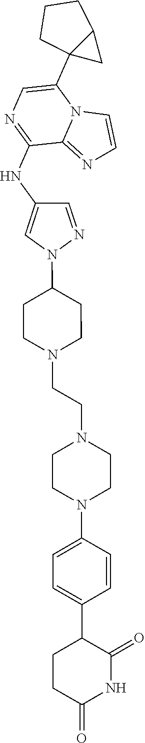 [SMILES: O=C1CCC(c2ccc(N3CCN(CCN4CCC(n5cc(Nc6ncc(C78CCCC7C8)n7ccnc67)cn5)CC4)CC3)cc2)C(=O)N1] | 3-(4-(4-(2-(4-(4-((5- (bicyclo[3.1.0]hexan-1- yl)imidazo[1,2-a]pyrazin- 8-yl)amino)-1H-pyrazol-1- yl)piperidin-1- yl)ethyl)piperazin-1- yl)phenyl)piperidine-2,6- dione | 16.5 mg, 8% yield (3 steps), white solid [M + H]⁺ = 663.4 ¹H NMR (400 MHz, DMSO-d6) δ ppm 0.76-0.82 (m, 1 H), 0.97- 1.01 (m, 1 H), 1.37- 1.46 (m, 1 H), 1.66-1.71 (m, 1 H), 1.72-1.78 (m, 1 H), 1.79-1.92 (m, 5 H), 1.94- 2.04 (m, 4 H), 2.05- 2.19 (m, 6 H), 2.55-2.59 (m, 3 H), 2.98-3.03 (m, 3 H), 3.08-3.14 (m, 6 H), 3.72- 3.75 (m, 1 H), 4.07- 4.14 (m, 1 H), 6.89 (br d, J = 9.0 Hz, 2 H), 7.05 (br d, J = 8.3 Hz, 2 H), 7.31 (s, 1 H), 7.64 (s, 1 H), 7.75 (s, 1 H), 7.85 (s, 1 H), 8.17 (br s, 1 H), 8.19 (s, 2 H), 9.66 (s, 1 H), 10.77 (s, 1 H). Three protons not apparent in the 1H NMR spectrum. |
| TBM-22 | CBM-2 | 90 | 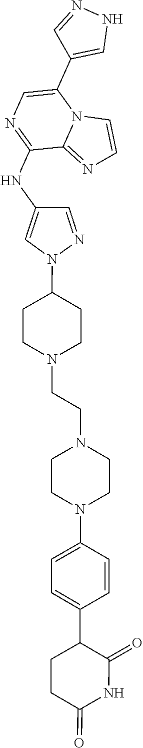 [SMILES: O=C1CCC(c2ccc(N3CCN(CCN4CCC(n5cc(Nc6ncc(-c7cn[nH]c7)n7ccnc67)cn5)CC4)CC3)cc2)C(=O)N1] | 3-(4-(4-(2-(4-(4-((5-(1H- pyrazol-4-yl)imidazo[1,2- alpyrazin-8-y1)amino)-1H- pyrazol-1-yl)piperidin-1- yl)ethyl)piperazin-1- y1)phenyl)piperidine-2,6- dione | 48 mg, 22% yield (3 steps), off-white solid [M + H]⁺ = 649.3 ¹H NMR (400 MHz, DMSO-d6) δ ppm 1.86-2.05 (m, 5 H), 2.08- 2.22 (m, 3 H), 2.41- 2.48 (m, 1 H), 2.52-2.66 (m, 7 H), 2.98-3.06 (m, 2 H), 3.08-3.15 (m, 4 H), 3.72 (dd, J = 11.0, 4.9 Hz, 1 H), 4.06-4.20 (m, 1 H), 6.89 (d, J = 8.8 Hz, 2 H), 7.05 (d, J = 8.8 Hz, 2 H), 7.56 (s, 1 H), 7.67, (d, J = 1.2 Hz, 1 H), 7.78 (s, 1 H), 7.96-8.39 (m, 5 H), 9.77 (s, 1 H), 10.77 (s, 1 H), |
| TBM-24 | CBM-2 | 111 | 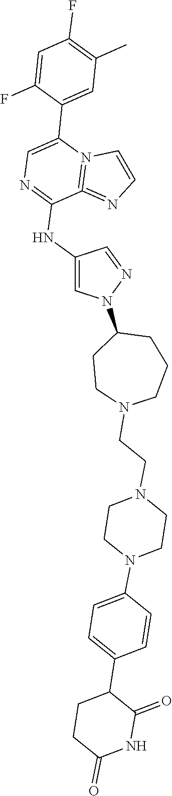 | 3-(4-(4-(2-((S)-4-(4-((5- (2,4-difluoro-5- methylphenyl)imidazo[1,2- a]pyrazin-8-yl)amino)-1H- pyrazol-1-yl)azepan-1- yl)ethyl)piperazin-1- yl)phenyl)piperidine-2,6- dione* *arbitrarily assigned as (S) | 13.31 (br s, 1 H). 33 mg, 6% yield (3 steps), off-white solid [M + H]⁺ = 723.4. ¹H NMR (400 MHz, DMSO-d6) δ ppm 1.59-1.68 (m, 1 H), 1.76- 1.83 (m, 1 H), 1.97- 2.15 (m, 8 H), 2.29 (s, 3 H), 2.54- 2.57 (m, 4 H), 2.63-2.72 (m, 7 H), 2.76-2.82 (m, 1 H), 3.09-3.12 (m, 4 H), 3.72 (dd, J = 11.1, 5.3 Hz, 1 H), 4.39-4.46 (m, 1 H), 6.88 (d, J = 8.8 Hz, 2 H), 7.03 (d, J = 8.8 Hz, 2 H), 7.42-7.47 (m, 2 H), 7.60-7.62 (m, 3 H), 7.77 (s, 1 H), 8.22 (s, 1 H), 9.97 (s, 1 H), 10.76 (s, 1 H). ¹⁹F NMR (377 MHz, DMSO-d6) δ ppm-112.15 (q, J = 8.6 Hz, 1 F), −111.26 (q, J = 8.6 Hz, 1 F). |
| TBM-25 | CBM-2 | 112 | 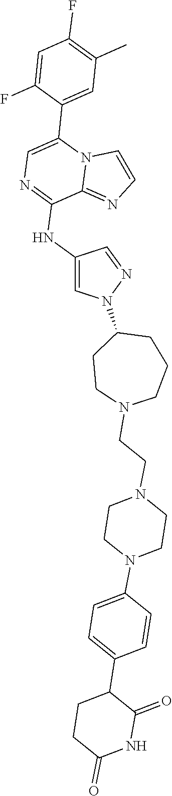 [SMILES: Cc1cc(-c2cnc(Nc3cnn([C@@H]4CCCN(CCN5CCN(c6ccc(C7CCC(=O)NC7=O)cc6)CC5)CC4)c3)c3nccn23)c(F)cc1F] | 3-(4-(4-(2-((R)-4-(4-((5- (2,4-difluoro-5- methylphenyl)imidazo[1,2- alpyrazin-8-yl)amino)-1H- pyrazol-1-yl)azepan-1- yl)ethyl)piperazin-1- yl)phenyl)piperidine-2,6- dione* *arbitrarily assigned as (R) | 25 mg, 8% yield (3 steps), off-white solid [M + H]⁺ = 723.4, [M + 2H]²⁺ = 362.3. ¹H NMR (400 MHz, DMSO-d6) δ ppm 1.57-1.70 (m, 2 H), 1.75- 1.85 (m, 2 H), 1.97- 2.08 (m, 5 H), 2.29 (s, 3 H), 2.47 (br s, 2 H), 2.56 (br s, 4 H), 2.61-2.73 (m, 6 H), 2.76- 2.83 (m, 1 H), 3.10 (br s, 4 H), 3.69- 3.75 (m, 1 H), 4.38- 4.45 (m, 1 H), 6.88 (d, J = 8.6 Hz, 2 H), 7.03 (d, J = 8.6 Hz, 2 H), 7.42- 7.48 (m, 2 H), 7.57- 7.64 (m, 3 H), 7.78 (s, 1 H), 8.22 (s, 1 H), 9.97 (s, 1 H), 10.77 (s, 1 H). |
| TBM-26 | CBM-2 | 114 | 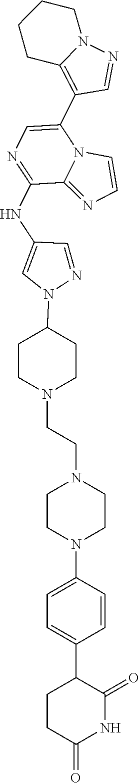 [SMILES: O=C1CCC(c2ccc(N3CCN(CCN4CCC(n5cc(Nc6ncc(-c7cnn8c7CCCC8)n7ccnc67)cn5)CC4)CC3)cc2)C(=O)N1] | 3-(4-(4-(2-(4-(4-((5- (4,5,6,7- tetrahydropyrazolo[1,5- a]pyridin-3- yl)imidazo[1,2-a]pyrazin- 8-y1)amino)-1H-pyrazol-1- yl)piperidin-1- yl)ethyl)piperazin-1- y1)phenyl)piperidine-2,6- dione | 41 mg, 14% yield (3 steps), white solid [M + H]⁺ = 703.4 ¹H NMR (400 MHz, DMSO-d6) δ ppm 1.76-1.85 (m, 2 H), 1.85- 2.07 (m, 8 H), 2.07- 2.18 (m, 3 H), 2.41-2.46 (m, 1 H), 2.53-2.65 (m, 5 H), 2.75 (t, J = 6.2 Hz, 2 H), 2.95- 3.04 (m, 2 H), 3.06- 3.15 (m, 4 H), 3.72 (dd, J = 11.0, 4.9 Hz, 1 H), 4.05- 4.21 (m, 3 H), 6.89 (d, J = 8.8 Hz, 2 H), 7.04 (d, J = 8.8 Hz, 2 H), 7.31 (s, 1 H), 7.61 (d, J = 1.0 Hz, 1 H), 7.74- 7.82 (m, 3 H), 8.23 (s, 1 H), 9.78 (s, 1 H), 10.77 (s, 1 H). |

### Biological Examples

### Example B1. IRAK3-ePL Overexpressing Degradation Assay

Stable cell lines were generated by the following protocol. 3×10⁵ Lenti-X 293T cells (Clonetech) were plated in 0.8 mL of media in a 12-well plate and incubated overnight at 37° C./5% CO₂. Packaging plasmid (0.4 μg, pMD), envelope plasmid (0.4 μg, pSP), and lentiviral transfer IRAK3-ePL plasmid (0.8 μg, TRAK3 sequence NM_007199.3) were mixed in 0.1 mL of Opti-MEM and incubated for 5 min. Simultaneously, 2.4 μL of Lipofectamine 2000 (Invitrogen) was added to 0.1 mL of Opti-MEM (Gibco) and incubated for 5 min. The plasmid DNA and lipofectamine were combined and the mixture was allowed to incubate for 20 min.DNA:Lipofectamine Opti-MEM mixtures were then added to previously plated cells dropwise and the cells were incubated for ~16 h at 37° C./5% CO₂. Following incubation, the media was removed and 1.2 mL of fresh media was added per well. Lenti-X 293T cells were incubated for ~30 h at 37° C./5% CO₂. 0.5×10⁶ 293T^{CRBN OE/GSPT1 G575N KI} cells were plated in 0.5 mL of media/well of a 12-well plate and incubated for ~16 h at 37° C./5% CO₂. Following incubation, media was removed from the Lenti-X 293T wells and passed through a 0.45 μM filter. Part of the viral supernatant was used to transduce cells and the rest was stored at −80° C. Viruses were then added individually (0.5 mL virus) to each well of the 293T^{CRBN OE/GSPT1 G575N KI} cells, followed by addition of polybrene (10 mg/mL Millipore) to each well at a final concentration of 5.0 μg/mL. Cells were incubated for ~24 h at 37° C./5% CO₂. After aspirating media off plates, cells were washed with DPBS, trypsinized and plated in a 10 cm dish in 15 mL of media and 1 g/mL puromycin. Following incubation of cells for ~72 h at 37° C./5% CO₂, media was aspirated off plates, and cells were washed with DPBS and trypsinized. The cells were plated in a 15 cm dish in 40 mL of media with 1.0 μg/mL Puromycin (Gibco) and incubated for ~72 h at 37° C./5% CO₂. Following incubation, media was removed and cells were washed with DPBS and trypsinized. The majority of cells were resuspended in Invitrogen freezing media and stored away (~6-8×10⁶ cells/vial).

IRAK3-ePL cellular dose response curve degradation assays were performed by the following protocol. Compounds to be tested were dispensed into a white 384-well tissue-culture treated plate using an acoustic liquid handler. Dilutions were prepared based on a 25 μL assay volume in duplicate 10 point 3-fold serial dilutions starting with a 10 μM dose. Negative control wells were included, which only contain 0.2% DMSO to calculate 100% signal. Positive control wells containing 30 μM Ataluren (luciferase inhibitor) were included to calculate the background signal level. All wells were backfilled to a final DMSO concentration of 0.2% to ensure DMSO uniformity across wells. IRAK3-ePL expressing cells (IRAK3-ePL Lenti-X 293T^{CRBN/GSPT1 G575N}) were washed, trypsinized, counted, and resuspended in fresh DMEM (Gibco) to give a cell concentration of 200,000 cells/mL. 25 μL of cells (5,000 cells/well) were dispensed into the wells of the 384-well plate prespotted with compounds in the previous step and incubated overnight at 37° C./5% CO₂. Following incubation, the 384-well plate was taken out of the incubator and left at room temperature for 30 min. InCELL hunter reagent was prepared according to manufacturer's instructions (EA reagent, lysis buffer, and substrate reagent in a 1:1:4 ratio, Cat #96-0002, DiscoverX), which was added to the 384-well plate 25 μL per well. Following the incubation of the plate for 1 h at room temperature, the luminescence signal was read using a ViewLux plate reader. Data was processed and analyzed in ActivityBase software. In short, the average luminescence values of the positive control wells were subtracted from the rest of the wells for background correction, and all luminescence values were normalized to the DMSO control wells. The average value of the DMSO control wells was set to equal 100% of the relative IRAK3-ePL protein levels. Normalized luminescence values were plotted on a graph as a function of compound concentration. Compound concentration was plotted on the x-axis and the corresponding relative IRAK3-ePL protein levels on the y-axis. The EC₅₀ value (the half-maximum effective concentration) of a compound for the degradation of the IRAK3-ePL was calculated using a four-parameter logistic model (sigmoidal dose-response model) (FIT=(A+{(B−A)/1+[(C/x)^{D}]})) where C is the inflection point (EC₅₀), D is the correlation coefficient, and A and B are the low and high limits of the fit, respectively).

The Dₘₐₓ was calculated by determining the maximum percentage loss of target protein following compound treatment. The Yₘᵢₙ was calculated by determining the lowest percentage of target protein remaining following compound treatment ((% Dₘₐₓ=100−Yₘᵢₙ).

### Example B₂. IRAK Endogenous HTRF Degradation Assay

Cells (~50 k) were plated in Cisbio 96-well low volume white plates (Cisbio: cat #66PL96005). Compounds were dissolved in DMSO and a 3-fold serial dilution was performed using a TECAN D300E. Cells were incubated with the compound overnight. Total-IRAK3 HTRF kit from Cisbio was used for degradation analysis (Cisbio: 63ADK101PEH). Cryptate and D2 antibodies were diluted in Detection buffer as per manufacturers recommendation. Then 2 μL of each solution was added to 16 μL lysate. Buffer control (lysis buffer detection buffer), Cryptate control (lysis buffer+cryptate antibody+detection buffer), and Negative control (lysis buffer+cryptate antibody+D2 antibody) were made as per manufacturers recommendation. Post incubation with antibodies the HTRF signal was measured using a Perkin Elmer Envision reader and the HTRF signal was calculated using formula: (Emission at 665 nm/Emission at 615 nm)*10,000. All HTRF values were normalized to the average value of DMSO. The average value of the DMSO control wells were set to equal 100% of the relative IRAK3 protein levels. Normalized luminescence values were plotted on a graph as a function of compound concentration. Compound concentration was plotted on the x-axis and the corresponding normalized IRAK3 protein levels on the y-axis. The EC₅₀ value (the half-maximum effective concentration) of a compound for the degradation of the IRAK3 was calculated using a four-parameter logistic model (sigmoidal dose-response model) (FIT=(A+{(B−A)/1+[(C/x)D]})) where C is the inflection point (EC₅₀), D is the correlation coefficient, and A and B are the low and high limits of the fit, respectively). The Yₘᵢₙ was calculated by determining the lowest percentage of target protein remaining following compound treatment. Dₘₐₓ was calculated from Yₘᵢₙ (% Dₘₐₓ=100−Yₘᵢₙ).

### Example B3. IRAK3 Biochemical Binding Assay

The LanthaScreen® Eu Kinase Binding assay was performed as described by the vendor (ThermoFisher Scientific Waltham, MA). Briefly, 100× solutions of compound were prepared in DMSO via serial dilution of the 10 mM stock solution in a 384-well reagent plate using 3-fold intervals to achieve final concentrations. 1 μL of the compound dilution series were added to the corresponding wells of a 384-well reagent plate containing 32.3 μL of 1× buffer (50 mM HEPES pH 7.4, 10 nM MgCl₂, 1 mM EGTA, 0.01% Brij-35). 5 μL of the buffer diluted compounds were transferred to the corresponding wells of a 384-well assay plate. 5 μL of 3× tracer was transferred to each well of the assay plate for a final tracer concentration of 10 nM. Finally, 5 μL of the 3×Eu-Anti-GST and IRAK3 mix was transferred to each well for a final concentration of 2 nM and 10 nM respectively. Reactions were allowed to incubate for 1 hour at room temperature. TR-FRET signal of the interaction (λₑₓ340/λₑₘ 665/λₑₘ 615) was read at room temperature with a delay time of 100 ρs and an integration time of 200 ρs using an Envision plate reader. Background corrected emission signal ratios at each compound concentration were used to calculate percentage of inhibition (% Inhibition). Plots of % Inhibition versus inhibitor concentrations were fit according to a dose-response equation (Eq. 1) to generate IC₅₀ and Hill slope values using Dotmatics software (Dotmatics, Bishops Stortford, Hertfordshire, England).

$$\% Inhibition = ( \frac{100}{1 + {( \frac{{IC}_{50}}{[ I ]} )}^{n}} ) \qquad ( Eq . 1 )$$

Using these assays, IC₅₀, Dₘₐₓ, EC₅₀, and DC₅₀ values of the following compounds were determined. Dₘₐₓ is defined as the maximum percent degradation achieved and DC₅₀ is the concentration at which 50% degradation is achieved. Data is summarized in Table 42.

TABLE 42

| | IRAK3 | | | | |
|---|---|---|---|---|---|
| Compound | binding | IRAK3 ePL degradation | | IRAK3 HTRF degradation | |
| No. | IC₅₀ (nM) | Yₘᵢₙ (%) | EC₅₀ (μM) | Yₘᵢₙ (%) | EC₅₀ (nM) |
| 1 | 5.0 | 31 | 0.039 | 27 | 0.015 |
| 2 | 4.2 | 15 | 0.003 | 18 | 0.074 |
| 3 | 1.3 | 15 | 0.003 | 11 | 0.016 |
| 4 | 2.3 | 13 | 0.004 | 17 | 0.025 |
| 5 | 5.9 | 14 | 0.0010 | ND | ND |
| 6 | 1.0 | 17 | 0.022 | 23 | 0.002 |
| 7 | 1.5 | 14 | 0.0006 | 12 | 0.0005 |
| 8 | 1.3 | 18 | 0.0005 | 9 | 0.002 |
| 9 | 1.1 | 14 | 0.0005 | 14 | 0.0006 |
| 10 | 0.51 | 22 | 0.019 | 14 | 0.0009 |
| 11 | 8.9 | 22 | 0.004 | 12 | 0.011 |
| 12 | 2.0 | 14 | 0.003 | 13 | 0.004 |
| 13 | 1.3 | 13 | 0.00086 | 11 | 0.00034 |
| 14 | 4.3 | 15 | 0.0011 | 13 | 0.023 |
| 15 | 6.4 | 15 | 0.00051 | ND | ND |
| 16 | 1.5 | 33 | 0.021 | 45 | 0.011 |
| 17 | 1.3 | 14 | 0.00051 | 15 | 0.003 |
| 18 | 0.5 | 22 | 0.0097 | 20 | 0.006 |
| 19 | 1.8 | 18 | 0.012 | 18 | 0.0033 |
| 20 | 2.9 | 16 | 0.00077 | ND | ND |
| 21 | ND | ND | ND | 25 | 0.008 |
| 22 | ND | ND | ND | 24 | 0.0071 |
| 23 | ND | ND | ND | 17 | 0.006 |
| 24 | 4.6 | ND | ND | 25 | 0.0001 |
| 25 | 1.7 | ND | ND | 18 | 0.006 |
| 26 | 1.1 | ND | ND | 12 | 0.006 |
| 27 | 7.6 | ND | ND | 14 | 0.009 |
| 28 | 8.2 | ND | ND | 19 | 0.022 |
| 29 | ND | 24 | 0.00051 | 14 | 0.0008 |
| 30 | ND | 24 | 0.00051 | 14 | 0.0002 |
| 31 | 6.1 | 44 | 0.038 | 40 | 0.015 |
| 32 | 36 | 23 | 0.0014 | ND | ND |
| 33 | 12 | 24 | 0.0079 | 13 | 0.010 |
| 34 | 1.5 | 23 | 0.0017 | ND | ND |
| 35 | 0.65 | 24 | 0.00063 | 7.6 | 0.0028 |
| 36 | ND | 24 | 0.0011 | 9.8 | 0.0008 |
| 37 | ND | 20 | 0.0038 | 10 | <0.0001 |
| 38 | ND | 25 | 0.00051 | 9.1 | <0.0001 |
| 39 | ND | 32 | 0.00051 | 14. | 0.0019 |
| 40 | ND | 24 | 0.0015 | 8.5 | 0.017 |
| 41 | ND | 25 | 0.00068 | 8.7 | 0.001 |
| 42 | ND | 23 | 0.00051 | 9.8 | 0.001 |
| 43 | ND | 24 | 0.0028 | ND | ND |
| 44 | ND | 26 | 0.00097 | 8.1 | 0.004 |
| 45 | ND | 26 | 0.0072 | 15 | 0.004 |
| 46 | ND | 24 | 0.00051 | ND | ND |
| 47 | ND | 23 | 0.00061 | ND | ND |
| 48 | ND | 24 | 0.00077 | ND | ND |
| 49 | ND | 24 | 0.0024 | ND | ND |
| 50 | ND | 25 | 0.0013 | 9.8 | 0.007 |
| 51 | ND | 24 | 0.0025 | 9.3 | 0.000 |
| 52 | ND | 40 | 0.096 | 74 | 0.715 |
| 53 | ND | 22 | 0.0009 | 11 | 0.003 |
| 54 | ND | 28 | 0.010 | 15 | 0.061 |
| 55 | ND | 84 | 1.10 | 64 | 0.328 |
| 56 | ND | 23 | 0.00075 | 8.6 | 0.001 |
| 57 | ND | 24 | 0.00570 | 8.72 | 0.044 |
| 58 | ND | 25 | 0.00098 | 9.7 | 0.005 |
| 59 | ND | 21 | 0.00051 | 7.5 | 0.001 |
| 60 | ND | 92 | 0.00051 | 96 | 1.12 |
| 61 | ND | 41 | 0.018 | 20 | 0.103 |
| 62 | ND | ND | ND | ND | ND |
| 63 | ND | 25 | 0.00051 | 8.9 | 0.009 |
| 64 | ND | 25 | 0.0042 | 8.6 | 0.033 |
| 65 | ND | 86 | 3.30 | 96 | 0.0007 |
| 66 | ND | 32 | 0.0096 | ND | ND |
| 67 | ND | 38 | 0.0055 | ND | ND |
| 68 | ND | 24 | 0.00054 | ND | ND |
| 69 | ND | ND | ND | ND | ND |
| 70 | ND | ND | ND | ND | ND |
| 71 | 2.8 | 29 | 0.0017 | 13 | 0.007 |
| 72 | 1.2 | 24 | 0.00051 | 8.3 | 0.006 |
| 73 | ND | 24 | 0.00051 | 9.1 | 0.004 |
| 74 | 5.6 | 26 | 0.0010 | 9.8 | 0.006 |
| 75 | 14 | 25 | 0.0041 | 9.8 | 0.006 |
| 76 | 1.7 | 78 | 10.0 | 64 | 0.353 |
| 77 | 2.0 | 27 | 0.0011 | 8.3 | 0.009 |
| 78 | 27 | 29 | 0.0055 | ND | ND |
| 79 | 260 | 29 | 0.029 | ND | ND |
| 80 | 140 | 28 | 0.026 | 11 | 0.178 |
| 81 | 10 | 27 | 0.0033 | 7.8 | 0.016 |
| 82 | 17 | 66 | 10.0 | 40 | 1.82 |
| 83 | 1600 | 90 | 10.0 | ND | ND |
| 84 | 24 | ND | ND | ND | ND |
| 85 | 25 | ND | ND | ND | ND |
| 86 | 380 | ND | ND | ND | ND |
| 87 | 4.0 | ND | ND | ND | ND |
| 88 | 16 | ND | ND | ND | ND |
| 89 | ND | ND | ND | ND | ND |
| 90 | ND | ND | ND | ND | ND |
| 91 | ND | ND | ND | ND | ND |
| 92 | ND | ND | ND | ND | ND |
| 93 | ND | ND | ND | ND | ND |
| 94 | ND | ND | ND | ND | ND |
| 95 | 15 | ND | ND | ND | ND |
| 96 | 0.22 | ND | ND | ND | ND |
| 97 | 1.3 | ND | ND | ND | ND |
| 98 | 0.08 | ND | ND | ND | ND |
| 99 | 0.44 | ND | ND | ND | ND |
| 100 | 4.0 | ND | ND | ND | ND |
| 101 | 7.6 | ND | ND | ND | ND |
| 102 | 0.80 | ND | ND | ND | ND |
| 103 | 2.0 | ND | ND | ND | ND |
| 104 | 0.30 | ND | ND | ND | ND |
| 105 | 0.18 | ND | ND | ND | ND |
| 106 | ND | ND | ND | ND | ND |
| 107 | ND | ND | ND | ND | ND |
| 108 | 2.6 | ND | ND | ND | ND |
| 109 | 0.26 | ND | ND | ND | ND |
| 110 | 0.86 | ND | ND | ND | ND |
| 111 | 0.05 | ND | ND | ND | ND |
| 112 | 0.48 | ND | ND | ND | ND |
| 113 | 3.2 | ND | ND | ND | ND |
| 114 | 0.92 | ND | ND | ND | ND |
| 115 | 25 | ND | ND | ND | ND |
| 116 | 0.12 | ND | ND | ND | ND |
| 117 | 1.0 | ND | ND | ND | ND |
| 118 | 0.10 | ND | ND | ND | ND |
| 119 | 1.70 | ND | ND | ND | ND |
| 120 | 1.5 | ND | ND | ND | ND |

ND = not determined

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 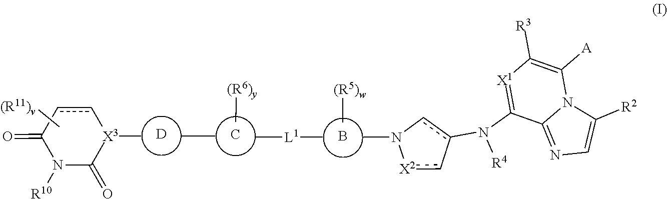
or a pharmaceutically acceptable salt thereof, wherein:
A is C₁-C₆ alkyl, phenyl, C₃-C₆ cycloalkyl, 5- to 6-membered heteroaryl, or 6- to 10-membered heterocyclyl, wherein the phenyl, cycloalkyl, heteroaryl, and heterocyclyl are substituted by x R¹ groups, and wherein the heteroaryl and heterocyclyl contain 1-3 heteroatoms selected from N and O;
each R¹ is independently halo, C₁-C₆ alkyl, C₃-C₆ cycloalkyl, C₁-C₆ alkoxy, C₁-C₆ haloalkyl, or —SO₂(C₁-C₆ alkyl);
or two R¹ groups on adjacent carbon atoms are taken together to form a fused C₃-C₆ cycloalkyl or a fused 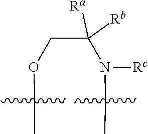
group;
R^{a} and R^{b} are each H or are taken together to form an oxo group;
R^{c} is H or C₁-C₆ alkyl;
x is 0-5;
R² is H or C₁-C₆ alkyl;
R³ is H or C₁-C₆ alkyl;
R⁴ is H or C₁-C₆ alkyl;
X¹ is CH or N;
X² is N or CH₂;
Ring B is C₃-C₆ cycloalkylene or 5- to 7-membered heterocyclylene containing 1 or 2 nitrogen atoms;
each R⁵ is independently halo, C₁-C₆ alkyl, or C₁-C₆ haloalkyl;
w is 0-5;
L¹ is —C(O)(CH₂)ₙ—, —(CH₂)ₙ—, or —(CH₂)ₙC(O)—;
n is 1-6;
Ring C is 5- to 10-membered heterocyclylene containing 1 or 2 nitrogen atoms;
each R⁶ is independently halo, C₁-C₆ haloalkyl, or C₁-C₆ alkyl;
y is 0-5;
Ring D is 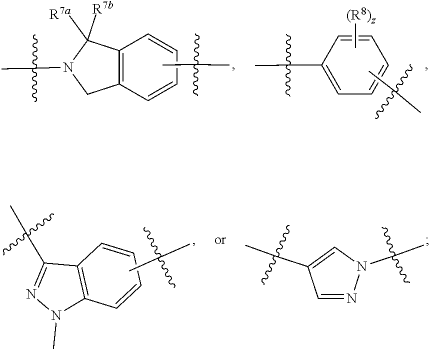
R^{7a} and R^{7b} are each H or are taken together to form an oxo group;
each R⁸ is independently halo, C₁-C₆ alkyl, C₁-C₆ haloalkyl, or C₁-C₆ alkoxy;
z is 0-4;
X³ is N or CR⁹;
R⁹ is H or C₁-C₆ alkyl;
R¹⁰ is H or C₁-C₆ alkyl;
each R¹¹ is independently halo, C₁-C₆ alkyl, or C₁-C₆ haloalkyl;
v is 0-4; and
each [symbol] is independently a single bond or double bond.

2. The compound of claim 1, or a pharmaceutically acceptable salt thereof, wherein:
A is C₁-C₃ alkyl, phenyl, C₃-C₅ cycloalkyl, 5- to 6-membered heteroaryl, or 8- to 10-membered heterocyclyl, wherein the phenyl, cycloalkyl, heteroaryl, and heterocyclyl are substituted by x R¹ groups;
x is 0-3; and
each R¹ is independently halo, C₁-C₃ alkyl, C₃-C₅ cycloalkyl, C₁-C₃ alkoxy, C₁-C₃ haloalkyl, or —SO₂(C₁-C₃ alkyl);
or two R¹ groups on adjacent carbon atoms are taken together to form a fused C₃-C₅ cycloalkyl or a fused 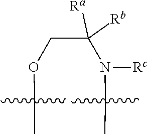
group;
R^{a} and R^{b} are each H or are taken together to form an oxo group; and
R^{c} is H or C₁-C₃ alkyl.

3. The compound of claim 1 or 2, or a pharmaceutically acceptable salt thereof, wherein:
A is 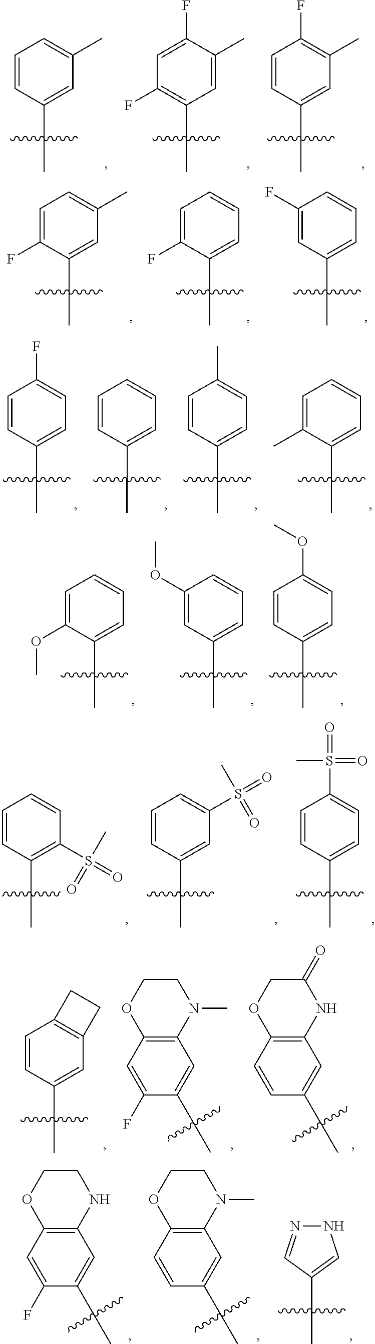 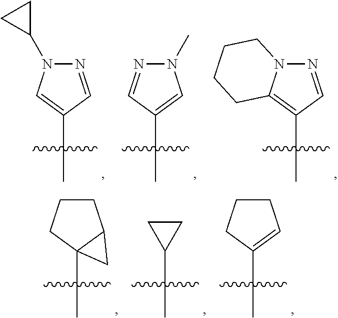
—CH₃, or —CH₂CH₃.

4. The compound of any one of claims 1-3, or a pharmaceutically acceptable salt thereof, wherein:
R² is H or C₁-C₃ alkyl;
R³ is H or C₁-C₃ alkyl; and
R⁴ is H or C₁-C₃ alkyl.

5. The compound of any one of claims 1-4, or a pharmaceutically acceptable salt thereof, wherein:
X¹ is N.

6. The compound of any one of claims 1-5, or a pharmaceutically acceptable salt thereof, wherein:

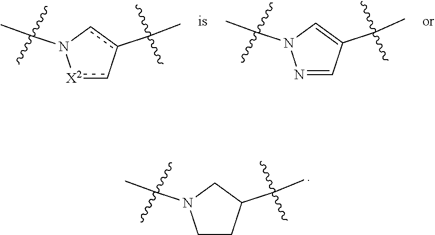

7. The compound of any one of claims 1-6, or a pharmaceutically acceptable salt thereof, wherein:
Ring B is C₄-C₆ cycloalkylene or 6- to 7-membered heterocyclylene containing one nitrogen atom;
w is 0-2; and
each R⁵ is independently halo, C₁-C₃ alkyl, or C₁-C₃ haloalkyl.

8. The compound of any one of claims 1-7, or a pharmaceutically acceptable salt thereof, wherein:

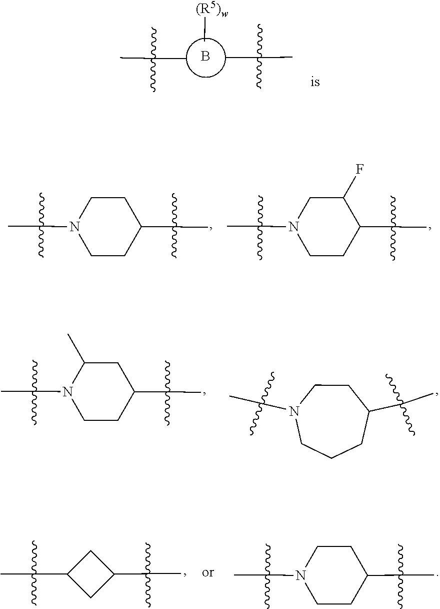

9. The compound of any one of claims 1-8, or a pharmaceutically acceptable salt thereof, wherein:
L¹ is —C(O)CH₂—, —(CH₂)ₙ—, or —CH₂C(O)—; and
n is 1-5.

10. The compound of any one of claims 1-9, or a pharmaceutically acceptable salt thereof, wherein:
Ring C is 6- to 8-membered heterocyclylene containing 1 or 2 nitrogen atoms;
y is 0-3; and
each R⁶ is independently halo, C₁-C₃ haloalkyl, or C₁-C₃ alkyl.

11. The compound of any one of claims 1-10, or a pharmaceutically acceptable salt thereof, wherein:

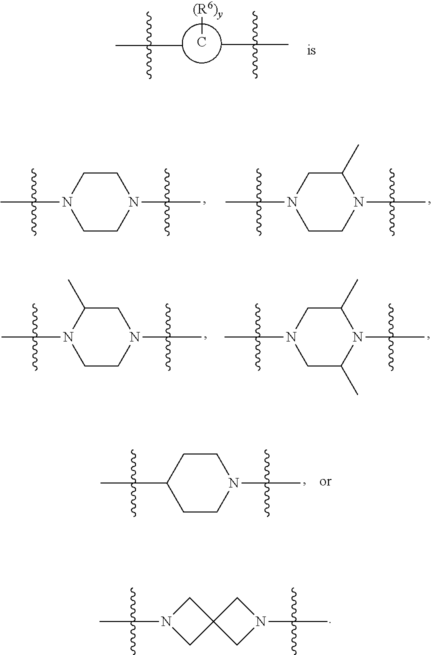

12. The compound of any one of claims 1-11, or a pharmaceutically acceptable salt thereof, wherein:
Ring D is

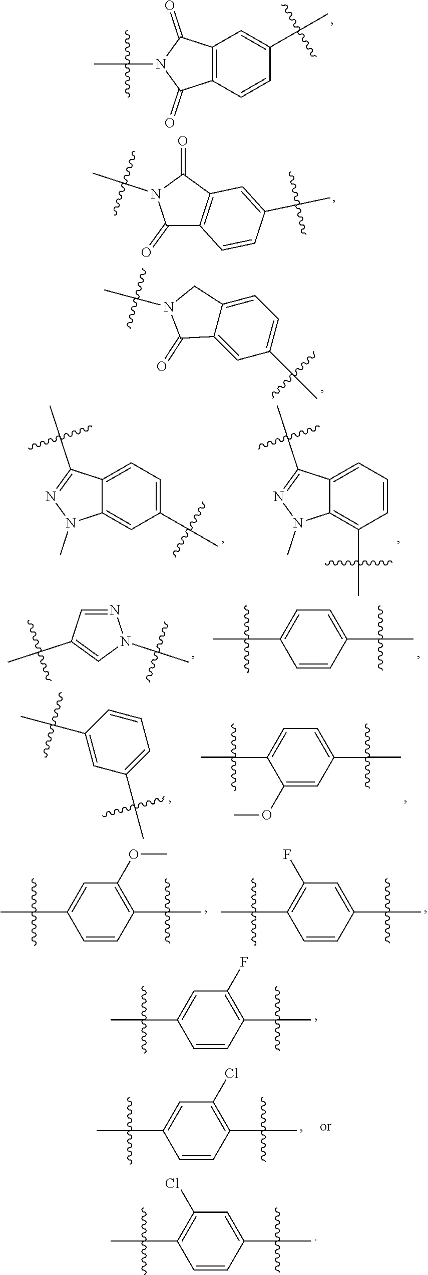

13. The compound of any one of claims 1-12, or a pharmaceutically acceptable salt thereof, wherein:
X³ is CR⁹;
R⁹ is H or C₁-C₃ alkyl;
R¹⁰ is H or C₁-C₃ alkyl;
v is 0-2; and
each R¹¹ is independently halo, C₁-C₃ alkyl, or C₁-C₃ haloalkyl.

14. The compound of any one of claims 1-13, or a pharmaceutically acceptable salt thereof, wherein:

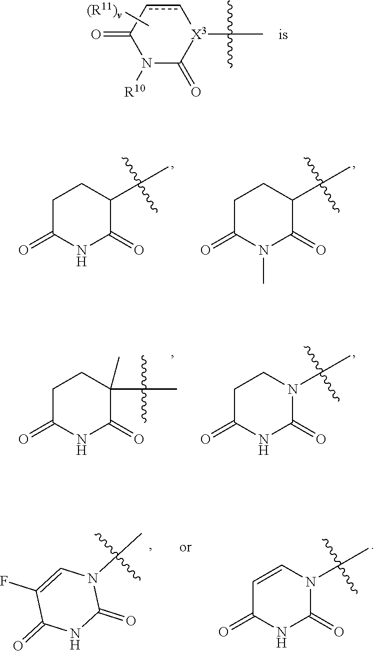

15. The compound of any one of claims 1-14, or a pharmaceutically acceptable salt thereof, wherein the compound is of Formula (IIIa), (IIIb), or (IIIc):

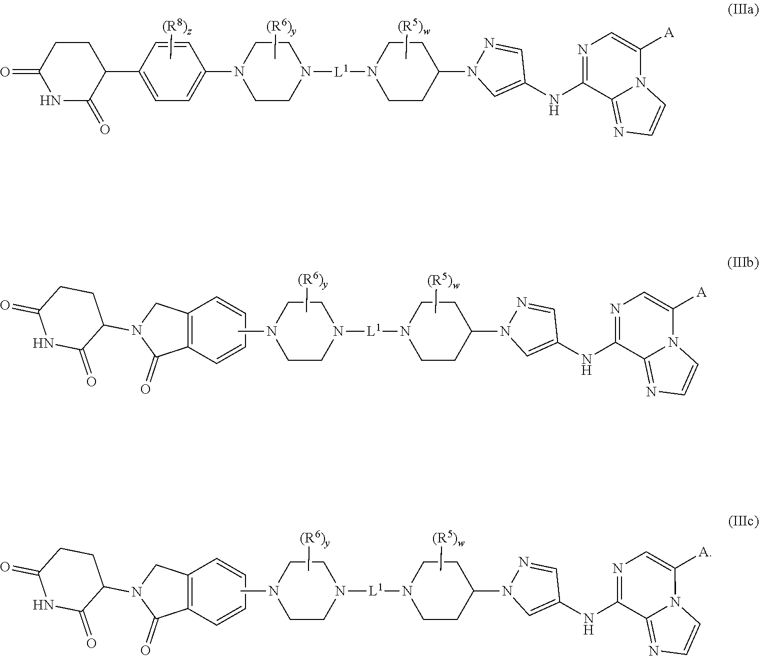

16. The compound of any one of claims 1-15, or a pharmaceutically acceptable salt thereof, wherein the compound is of Formula (IVa) or (IVb):

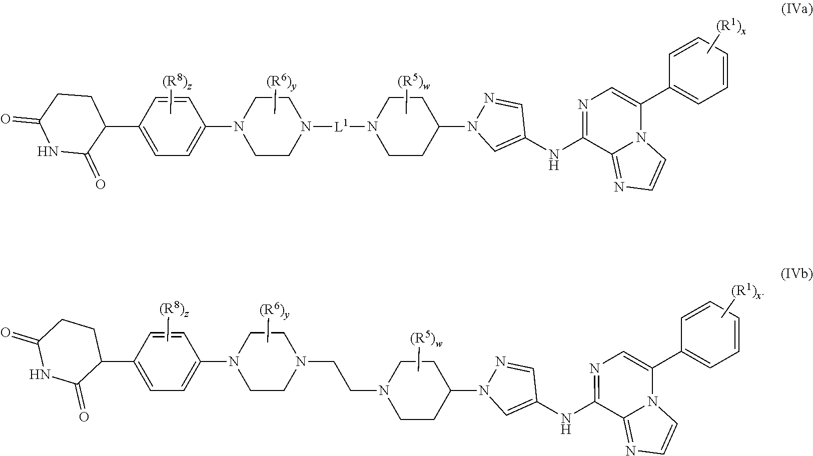

17. A compound selected from the compounds of Table 1 or a pharmaceutically acceptable salt thereof.

18. A pharmaceutical composition comprising the compound of any one of claims 1-17, or a pharmaceutically acceptable salt thereof, and a pharmaceutically acceptable excipient.

19. A method of modulating Interleukin-1 Receptor-Associated Kinase 3 (IRAK3) comprising contacting IRAK3 with an effective amount of the compound of any one of claims 1-17, or a pharmaceutically acceptable salt thereof, or the pharmaceutical composition of claim 18.

20. A method of (i) treating cancer in a subject in need thereof, comprising administering to the subject an effective amount of the compound of any one of claims 1-17, or a pharmaceutically acceptable salt thereof, or the pharmaceutical composition of claim 18, optionally wherein the cancer is selected from bladder cancer, breast cancer, esophgeal cancer, colon cancer, head and neck cancer, kidney cancer, lung cancer, pancreatic cancer, prostate cancer, melanoma, and gastric cancer; or (ii) enhancing immunity in a subject receiving a vaccine, comprising administering to the subject an effective amount of the compound of any one of claims 1-17, or a pharmaceutically acceptable salt thereof, or the pharmaceutical composition of claim 18.
